(19) United States
(12) Patent Application Publication
(10) Pub. No.: US 2022/0177872 A1
(43) Pub. Date: Jun. 9, 2022

(54) DEEP MUTATIONAL EVOLUTION OF BIOMOLECULES

(71) Applicant: Scribe Therapeutics Inc., Alameda, CA (US)

(72) Inventors: Benjamin OAKES, El Cerrito, CA (US); Sean HIGGINS, Alameda, CA (US); Hannah SPINNER, Boston, MA (US); Kian TAYLOR, Atlanta, GA (US); Sarah DENNY, San Francisco, CA (US)

(21) Appl. No.: 17/542,238

(22) Filed: Dec. 3, 2021

Related U.S. Application Data
(63) Continuation of application No. PCT/US2020/036506, filed on Jun. 5, 2020.
(60) Provisional application No. 62/858,718, filed on Jun. 7, 2019.

(51) Int. Cl. C12N 15/10; C12N 9/22; C12N 15/11; C12N 15/63; C12N 15/70; C12N 15/86

(52) U.S. Cl. CPC C12N 15/1058; C12N 15/1086; C12N 9/22; C12N 15/11; C12N 15/635; C12N 2800/101; C12N 15/86; C12N 2310/20; C12N 2800/80; C12N 2740/15043; C12N 15/70

(57) ABSTRACT

Provided herein are methods of developing biomolecule variants (such as proteins, RNA, or DNA) with improved characteristics, for example by developing libraries of variants with alterations to one or more specific monomer locations and screening said libraries for characteristics of interest. These alterations can include deletion, substitution, and insertion, and variants may comprise one alteration or a combination of alterations. Said methods may include further iterative cycles of library construction and evaluation to develop, for example, a biomolecule variant with improved characteristics compared to a reference biomolecule. The methods can also provide information that may be used in the rational design of variants.

### CROSS REFERENCE TO RELATED APPLICATIONS

This application is a continuation of International Patent Application No. PCT/US2020/036506, filed on Jun. 5, 2020, which claims priority to U.S. provisional patent application number 62,858,718, filed on Jun. 7, 2019, the contents of which are incorporated herein by reference in their entirety.

### INCORPORATION BY REFERENCE OF SEQUENCE LISTING

This application contains a Sequence listing which has been submitted in ASCII format via EFS-WEB and is hereby incorporated by reference in its entirety. Said ASCII copy, created on Dec. 3, 2021 is named SCRB_012_01_US_SeqList_ST25.txt and is 3.36 MB in size.

### BACKGROUND

Naturally occurring biomolecules, such as proteins, RNA, and DNA, often exist in a highly specific context and with specific functional requirements, which may not be optimal for other desired applications, such as research, biotechnological, and medical applications. Thus, mutation of biomolecules can be an important tool in modifying biomolecule structure and/or function. Typical modification techniques often target only a subset of the total biomolecule sequence, and also focus on one type of alteration, usually substitution of biomolecule monomers.

It is believed that insertions and deletions can be fundamental steps along the sequence-function landscape of a given biomolecule, in addition to standard substitution mutations. What is needed in the art are methods of evaluating a broad spectrum of different mutations at varying places along a biomolecule, and ways of combining such mutations, to obtain biomolecule variants with new or improved functionality.

### SUMMARY

In some aspects, provided herein is a method of selecting an improved biomolecule variant, wherein the biomolecule is a protein, DNA, or RNA, comprising:
(i) constructing a library comprising a plurality of biomolecule variants;
   wherein each variant is independently a variant of the same reference biomolecule, wherein each variant comprises an alteration of one or more monomer locations of the reference biomolecule, wherein the monomer is an amino acid of the protein or a ribonucleotide of the RNA or deoxyribonucleotide of the DNA,
   wherein each alteration of a monomer location is independently selected from the group consisting of substitution of the monomer, deletion of one or more consecutive monomers beginning at the location, and insertion of one or more consecutive monomers adjacent to the location; and
   wherein the library represents variants comprising alteration of one or more locations for at least 1% of the monomer locations of the reference biomolecule;
(ii) screening the library of (i);
(iii) identifying at least a portion of the library of (i) that exhibits one or more improved characteristics compared to the reference biomolecule; and
(iv) selecting the improved biomolecule variant from the at least a portion of the library, wherein the improved biomolecule variant exhibits one or more improved characteristics compared to the reference biomolecule.

In some embodiments, the portion of the library identified in step (iii) is screened. In some embodiments, the screen is a different screen than used in (ii), while in other embodiments it is the same screen.

In other aspects, provided herein is a method of selecting an improved biomolecule variant, wherein the biomolecule is a protein or RNA or DNA, comprising:
(i) constructing a library comprising a plurality of biomolecule variants;
   wherein each variant is independently a variant of the same reference biomolecule, wherein each variant comprises an alteration of one or more monomer locations of the reference biomolecule, wherein the monomer is an amino acid of the protein or ribonucleotide of the RNA or deoxyribonucleotide of the DNA,
   wherein each alteration of a monomer location is independently selected from the group consisting of substitution of the monomer, deletion of one or more consecutive monomers beginning at the location, and insertion of one or more consecutive monomers adjacent to the location; and
   wherein the library represents variants comprising alteration of one or more locations for at least 1% of the monomer locations of the reference biomolecule;
(ii) screening the library of (i);
(iii) identifying at least a portion of the library of (i) that exhibits one or more improved characteristics compared to the reference biomolecule;
(iv) carrying out one or more additional rounds of library construction and screening to produce a final library, wherein construction of each library comprises:
   altering one or more additional monomer locations of the identified portion of the previous library to produce a subsequent library of biomolecule variants;
(v) selecting the improved biomolecule variant from the final library of biomolecule variants, wherein the improved biomolecule variant exhibits one or more improved characteristics compared to the reference biomolecule.

In some embodiments of the methods provided herein, the library in step (i) comprises biomolecule variants with a single alteration of a single monomer location, biomolecule variants with a single alteration of two monomer locations, and biomolecule variants with a single alteration of three monomer locations, wherein each alteration is independently selected from the group consisting of substitution of the monomer, deletion of one or more consecutive monomers beginning at the location, and insertion of one or more consecutive monomers adjacent to the location. In certain embodiments, the methods comprise one, two, three, or more additional round of library construction and screening. In some embodiments, the improved biomolecule variant comprises an alteration of two or more, five or more, ten or more, or fifteen or more monomer locations of the reference biomolecule.

In some embodiments, the library in step (i) represents variants comprising a single alteration of a single location for at least 5%, at least 10%, at least 30%, at least 70%, or at least 90% of the total monomer locations. In other embodiments, each variant of the library in step (i) independently comprises alteration of one or more monomer locations, and the totality of the library represents variation of at least 5%, at least 10%, at least 30%, at least 70%, or at least 90% of the total monomer locations of the reference biomolecule.

In other aspects, provided herein is a method of constructing a library of polynucleotide variants of a reference biomolecule, comprising:
(a) constructing a polynucleotide that encodes for a variant of the reference biomolecule, wherein the reference biomolecule is a protein or RNA or DNA;
   wherein the polynucleotide encodes for an alteration of one or more monomer locations of the reference biomolecule, wherein the monomer is an amino acid of the protein or ribonucleotide of the RNA or deoxyribonucleotide of the DNA, and
   wherein each alteration of a monomer location is independently selected from the group consisting of substitution of the monomer, deletion of one or more consecutive monomers beginning at the location, and insertion of one or more consecutive monomers adjacent to the location; and
(b) repeating the polynucleotide construction of (a) a sufficient number of times such that the library of polynucleotide represents variants comprising a single alteration of a single location for at least 1% of the monomer locations of the biomolecule.

In still further aspects, provided herein is a polynucleotide variant library, comprising polynucleotide variants of a reference biomolecule, comprising:
a plurality of polynucleotides that independently encode for a variant of the reference biomolecule, wherein the reference biomolecule is a protein or RNA or DNA;
   wherein each polynucleotide independently encodes an alteration of one or more monomer locations of the reference biomolecule, wherein the monomer is an amino acid of the protein or ribonucleotide of the RNA or deoxyribonucleotide of the DNA, and
   wherein each alteration of a monomer location is independently selected from the group consisting of substitution of the monomer, deletion of one or more consecutive monomers beginning at the location, and insertion of one or more consecutive monomers adjacent to the location; and
   wherein the library of polynucleotides represents variants comprising a single alteration of a single location for at least 1% of the monomer locations.

In some embodiments of the methods provided herein, the library of polynucleotides represents variants comprising a single alteration of a single location for at least 5%, at least 10%, at least 30%, at least 70%, or at least 90% of the total monomer locations. In other embodiments, each variant comprises alteration of one or more locations, and the totality of the library represents variation of at least 5%, at least 10%, at least 30%, at least 70%, or at least 90% of the total monomer locations of the reference biomolecule.

In some embodiments of the methods provided herein, the library of polynucleotides represents variants comprising substitution of the monomer, variants comprising deletion of one or more monomers beginning at the location, and variants comprising insertion of one or more new monomers adjacent to the location for at least 10% of monomer locations. In some embodiments, for each inserted new monomer, the library of polynucleotides represents each naturally occurring monomer possibility.

In some embodiments, the library of polynucleotides represents variants for each of the following alterations for at least 80% of the monomer locations:
deletion of each of one, two, three, and four consecutive monomers,
insertion of each of one, two three, and four consecutive monomers, and
substitution of the same monomer with each of the other naturally occurring monomers.

In still further aspects, provided herein is a vector library comprising a plurality of vectors, wherein each vector independently comprises one polynucleotide of a polynucleotide variant library as described herein, and wherein the vector library collectively comprises the variant library. In some embodiments, vectors are bacterial plasmids. In certain embodiments, the vectors are constructed with plasmid recombineering.

In still further aspects, provided herein is a method of selecting a biomolecule variant, comprising:
producing a library of reference biomolecule variants from a polynucleotide variant library as described herein, or a vector library as described herein;
screening the library of reference biomolecule variants for one or more functional characteristics; and
selecting a biomolecule variant from the library of reference biomolecule variants.

In some embodiments, the one or more functional characteristics is selected from the group consisting of binding, activity, editing efficiency, editing specificity, and off-target cleavage. In certain embodiments, the screening comprises ranking the one or more functional characteristics for each of at least a portion of the biomolecule variants. In still further embodiments, the screening comprises deep sequencing of at least a portion of the plurality of polynucleotides.

In yet further aspects, provided herein is a biomolecule variant selected by any of the methods described herein. In some embodiments, the biomolecule variant has one or more improved functional characteristics compared to the reference biomolecule. In certain embodiments, one or more improved functional characteristics is selected from the group consisting of binding, activity, editing efficiency, editing specificity, and off-target cleavage. In some embodiments, the improvement is at least 1.1 fold, at least 1.5 fold, at least 10 fold, or between 1.5 to 100 fold.

In other aspects, provided herein is a library of variant oligonucleotides, wherein:
each variant oligonucleotide independently encodes an alteration of one or more sequential monomer locations of a reference biomolecule, wherein:
   the reference biomolecule is a protein or RNA or DNA,
   the one or more monomers are one or more amino acids of the protein or ribonucleotides of the RNA or deoxyribonucleotides of the DNA, and
   wherein each alteration of a monomer location is independently selected from the group consisting of substitution of the monomer, deletion of one or more consecutive monomers beginning at the location, and insertion of one or more consecutive monomers adjacent to the location;
each variant oligonucleotide comprises a pair of homology arms flanking the encoded alteration, wherein the homology arms are homologous to the reference biomolecule sequences flanking the corresponding monomer location alteration, and wherein each homology arm independently comprises between 10 to 100 nucleotides; and
the library of variant oligonucleotides represents alteration of a single monomer for at least 80% of monomer locations.

In some embodiments, each variant oligonucleotide independently encodes an alteration of one monomer location of the reference biomolecule.

In yet other aspects, provided herein is a library comprising a plurality of RNA variants, wherein each variant is independently a variant of the same reference RNA, and each variant comprises a point mutation, deletion, or insertion at one ribonucleotide location of the reference RNA sequence; wherein the library represents variants comprising the single alteration of a single location, for at least 1% of the ribonucleotide locations of the reference RNA sequence. In some embodiments, the library represents variants comprising the single alteration of a single location, for at least 5%, at least 10%, at least 30%, at least 50%, or at least 80% of the ribonucleotide locations of the reference RNA sequence. In other embodiments, each variant comprises alteration of one or more ribonucleotide locations, and the totality of the library represents variation of at least 5%, at least 10%, at least 30%, at least 70%, or at least 90% of the total ribonucleotide locations of the reference RNA sequence.

In further aspects, provided herein is a library comprising a plurality of protein variants, wherein each variant is independently a variant of the same reference protein, and each variant comprises an amino acid substitution, deletion, or insertion at one amino acid location of the reference protein sequence; wherein the library represents variants comprising the single alteration of a single location, for at least 1% of the amino acids of the reference protein sequence. In some embodiments, the library represents variants comprising the single alteration of a single location, for at least 5%, at least 10%, at least 30%, at least 50%, or at least 80% of the amino acids of the reference protein sequence. In other embodiments, each variant comprises alteration of one or more amino acid locations, and the totality of the library represents variation of at least 5%, at least 10%, at least 30%, at least 70%, or at least 90% of the total amino acid locations of the reference protein.

In still further aspects, provided herein is a library comprising a plurality of DNA variants, wherein each variant is independently a variant of the same reference DNA, and each variant comprises a point mutation, deletion, or insertion at one deoxyribonucleotide location of the reference DNA sequence; wherein the library represents variants comprising the single alteration of a single location, for at least 1% of the deoxyribonucleotide locations of the reference DNA sequence. In some embodiments, the library represents variants comprising the single alteration of a single location, for at least 5%, at least 10%, at least 30%, at least 50%, or at least 80% of the deoxyribonucleotide locations of the reference DNA sequence. In other embodiments, each variant comprises alteration of one or more deoxyribonucleotide locations, and the totality of the library represents variation of at least 5%, at least 10%, at least 30%, at least 70%, or at least 90% of the total deoxyribonucleotide locations of the reference DNA.

In certain embodiments of the methods, compositions, and libraries provided herein, the reference biomolecule is a CRISPR associated protein. In certain embodiments, the CRISPR associated protein is CasX. In some embodiments, the one or more improved characteristics are independently selected from the group consisting of improved folding of the variant, improved binding affinity to the guide RNA, improved binding affinity to a target DNA, altered binding affinity to one or more PAM sequences, improved unwinding of a target DNA, increased activity, improved editing efficiency, improved editing specificity, increased activity of the nuclease, increased target strand loading for double strand cleavage, decreased target strand loading for single strand nicking, decreased off-target cleavage, decreased off-target binding/nicking, improved binding of the non-target strand of a DNA, improved protein stability, improved protein:guide-RNA complex stability, improved protein solubility, improved protein:guide-NA complex stability, improved protein yield, increased collateral activity, and decreased collateral activity.

In other embodiments of the methods, compositions, and libraries provided herein, the reference biomolecule is a CRISPR guide RNA. In some embodiments, the CRISPR guide RNA is a guide RNA that binds to CasX. In some embodiments, the one or more improved characteristics are independently selected from the group consisting of improved stability, improved solubility, improved resistance to nuclease activity, improved binding affinity to a reference CRISPR associated protein, improved binding affinity to a target DNA, improved gene editing, and improved specificity.

### DESCRIPTION OF THE FIGURES

The present application can be understood by reference to the following description taken in conjunction with the accompanying figures.

FIG. 1 is a diagram showing an exemplary method of making CasX protein and guide RNA variants of the disclosure using Deep Mutational Evolution (DME). In some exemplary embodiments, DME builds and tests nearly every possible mutation, insertion and deletion in a biomolecule and combinations/multiples thereof, and provides a near comprehensive and unbiased assessment of the fitness landscape of a biomolecule and paths in sequence space towards desired outcomes. As described herein, DME can be applied to both CasX protein and guide RNA.

FIG. 2 is a diagram and an example fluorescence activated cell sorting (FACS) plot illustrating an exemplary method for assaying the effectiveness of a reference CasX protein or single guide RNA (sgRNA), or variants thereof. A reporter (e.g. GFP reporter) coupled to a gRNA target sequence, complementary to the gRNA spacer, is integrated into a reporter cell line. Cells are transformed or transfected with a CasX protein and/or sgRNA variant, with the spacer motif of the sgRNA complementary to and targeting the gRNA target sequence of the reporter. Ability of the CasX:sgRNA ribonucleoprotein complex to cleave the target sequence is assayed by FACS. Cells that lose reporter expression indicate occurrence of CasX:sgRNA ribonucleoprotein complex-mediated cleavage and indel formation.

FIG. 3A and FIG. 3B are exemplary heat maps showing the results of an exemplary DME mutagenesis of the reference sgRNA encoded by SEQ ID NO: 5, as described in Example 3. FIG. 3A shows the effect of single base pair (single base) substitutions, double base pair (double base) substitutions, single base pair insertions, single base pair deletions, and a single base pair deletion plus at single base pair substitution at each position of the reference sgRNA shown at top. FIG. 3B shows the effect of double base pair insertions and a single base pair insertion plus a single base pair substitution at each position of the improved reference sgRNA. The reference sgRNA sequence is UACUGGCGCUUUUAUCUCAUUACUUUGAGAGCCAUCACCAGCGACUAUGUCGUA UGGGUAAAGCGCUUAUUUAUCGGAGAGAAAUCCGAUAAAUAAGAAGCAUCAAAG (SEQ ID NO: 5) and is shown at the top of FIG. 3A and bottom of FIG. 3B. In FIG. 3A and FIG. 3B, Log₂ fold enrichment of the variant in the DME library relative to the reference CasX sgRNA following selection is indicated in grayscale. The results show regions of the reference sgRNA that should not be mutated and key regions that should be targeted for mutagenesis.

FIG. 4A shows the results of exemplary DME experiments using a reference sgRNA, as described in Example 3. The improved reference sgNA (an sgRNA) with a sequence of SEQ ID NO: 5 is shown at top, and Log₂ fold enrichment of the variant in the DME library relative to the reference sgRNA following selection is indicated in grayscale. Enrichment is a proxy for activity, where greater enrichment is a more active molecule. The heat map shows an exemplary DME experiment showing four replicates of a library where every base pair in the reference sgRNA has been substituted with every possible alternative base pair.

FIG. 4B is a series of 8 plots that compare biological replicates of different DME libraries. The Log₂ fold enrichment of individual variants relative to the reference sgRNA sequence for pairs of DME replicates are plotted against each other. Shown are plots for single deletion, single insertion and single substitution DME experiments, as well as wild type controls, and the plots indicate that there is a good amount of agreement for each replicate.

FIG. 4C is a heat map of an exemplary DME experiment showing four replicates of a library where every location in the reference sgRNA has undergone a single base pair insertion. The DME experiment used a reference sgRNA of SEQ ID NO: 5 (at top), and was performed as described in Example 3. Log₂ fold enrichment of the variant in the DME library relative to the reference sgRNA following selection is indicated in grayscale.

FIGS. 5A-5E are a series of plots showing that sgNA variants can improve gene editing by greater than two fold in an EGFP disruption assay, as described in Examples 2 and 3. Editing was measured by indel formation and GFP disruption in HEK293 cells carrying a GFP reporter. FIG. 5A shows the fold change in editing efficiency of a CasX sgRNA reference of SEQ ID NO: 4 and a variant of the reference which has a sequence of SEQ ID NO: 5, across 10 targets. When averaged across 10 targets, the editing efficiency of sgRNA SEQ ID NO: 5 improved 176% compared to SEQ ID NO: 4. FIG. 5B shows that further improvement of the sgRNA scaffold of SEQ ID NO: 5 is possible by swapping the extended stem loop sequence for additional sequences to generate the scaffolds whose sequences are shown in Table 3. Fold change in editing efficiency is shown on the Y-axis. FIG. 5C is a plot showing the fold improvement of sgNA variants (including SEQ ID NO: 17) generated by DME mutations normalized to SEQ ID NO: 5 as the CasX reference sgRNA. FIG. 5D is a plot showing the fold improvement of sgNA variants of sequences listed in Table 3, which were generated by appending ribozyme sequences to the reference sgRNA sequence, normalized to SEQ ID NO: 5 as the CasX reference sgRNA. FIG. 5E is a plot showing the fold improvement normalized to the SEQ ID NO: 5 reference sgRNA of variants created by both combining (stacking) scaffold stem mutations showing improved cleavage, DME mutations showing improved cleavage, and using ribozyme appendages showing improved cleavage. The resulting sgNA variants yield 2 fold or greater improvement in cleavage compared to SEQ ID NO: 5 in this assay. EGFP editing assays were performed with spacer target sequences of E6 and E7.

FIG. 6 shows a Hepatitis Delta Virus (HDV) genomic ribozyme used in exemplary gNA variants (SEQ ID NOs: 18-22, from top to bottom and left to right).

FIGS. 7A-7I are a series of heat maps showing the effect of single amino acid substitutions, single amino acid insertions, and deletions at each amino acid position in a reference CasX protein of SEQ ID NO: 2, as described in Example 4. Data were generated by a DME assay run at 37° C. The Y-axis shows each possible substitution or insertion (from top to bottom: R, H, K, D, E, S, T, N, Q, C, G, P, A, I, L, M, F, W, Y, V; boxes indicate the amino acid identity of the reference protein), the X-axis shows the amino acid position in the reference CasX protein. Grayscale indicates log₂ fold enrichment of the CasX variant protein relative to the reference CasX protein of SEQ ID NO: 2 in a DME library following enrichment. As used herein, “enrichment” is a proxy for activity, where greater enrichment is a more active molecule. (*)s indicate active sites. FIGS. 7A-7D show the effect of single amino acid substitutions. FIGS. 7E-7H show the effect of single amino acid insertions. FIG. 7I shows the effect of single amino acid deletions.

FIGS. 8A-8C are a series of heat maps showing the effect of single amino acid substitutions, single amino acid insertions and deletions at each amino acid position in a reference CasX protein of SEQ ID NO: 2, as described in Example 4. Data were generated by a DME assay run at 45° C. FIG. 8A shows the effect of single amino acid substitutions. FIG. 8B shows the effect of single amino acid insertions. FIG. 8C shows the effect of single amino acid deletions. For all of FIGS. 8A-8C, The Y-axis shows each possible substitution or insertion (from top to bottom: R, H, K, D, E, S, T, N, Q, C, G, P, A, 1, L, M, F, W, Y, V; boxes indicate the amino acid identity of the reference protein), the X-axis shows the amino acid position in the reference CasX protein. Grayscale indicates log₂ fold enrichment of the CasX variant protein relative to the reference CasX protein of SEQ ID NO: 2 in a DME library following enrichment. Enrichment may be thought of as a proxy for activity, where greater enrichment is a more active molecule. (*)s indicate active sites. Running this assay at 45° C. enriches for different variants than running the same assay at 37° C. (see FIGS. 7A-7I), thereby indicating which amino acid residues and changes are important for thermostability and folding.

FIG. 9 shows a survey of the comprehensive mutational landscape of all single mutations of a reference CasX protein of SEQ ID NO: 2, as described in Example 4. On the Y-axis, fold enrichment of CasX variants relative to the reference CasX protein for single substitutions (top), single insertions (middle) or single deletions (bottom). On the X-axis, amino acid position in the reference CasX protein. Key regions that yield improved CasX variants are the initial helix region and regions in the RuvC domain bordering the target strand loading (TLS) domain, as well as others.

FIG. 10 is a plot showing that the evaluated CasX variant proteins improved editing greater than three-fold relative to a reference CasX protein in the EGFP disruption assay, as described in Example 5. CasX proteins were tested for their ability to cleave an EGFP reporter at 2 different target sites in human HEK293 cells, and the normalized improvement in genome editing at these sites over the basic reference CasX protein of SEQ ID NO: 2 is shown. Variants, from left to right (indicated by the amino acid substitution, insertion or deletion at the given residue number) are: Y789T, [P793], Y789D, T72S, I546V, E552A, A636D, F536S, A708K, Y797L, L792G, A739V, G791M, \hat{ }G661, A788W, K390R, A751S, E385A, \hat{ }P696, \hat{ }M773, G695H, \hat{ }AS793, \hat{ }AS795, C477R, C477K, C479A, C479L, I55F, K210R, C233S, D231N, Q338E, Q338R, L379R, K390R, L481Q, F495S, D600N, T886K, A739V, K460N, I199F, G492P, T1531, R591I, \hat{ }AS795, \hat{ }AS796, \hat{ }L889, E121D, S270W, E712Q, K942Q, E552K, K25Q, N47D, \hat{ }T696, L685I, N880D, Q102R, M734K, A724S, T704K, P224K, K25R, M29E, H152D, S219R, E475K, G226R, A377K, E480K, K416E, H164R, K767R, I7F, M29R, H435R, E385Q, E385K, I279F, D489S, D732N, A739T, W885R, E53K, A238T, P283Q, E292K, Q628E, R388Q, G791M, L792K, L792E, M779N, G27D, K955R, S867R, R693I, F189Y, V635M, F399L, E498K, E386S, V254G, P793S, K188E, QT945KI, T620P, T946P, TT949PP, N952T, K682E, K975R, L212P, E292R, 1303K, C349E, E385P, E386N, D387K, L404K, E466H, C477Q, C477H, C479A, D659H, T806V, K808S, \hat{ }AS797, V959M, K975Q, W974G, A708Q, V711K, D733T, L742W, V747K, F755M, M771A, M771Q, W782Q, G791F, L792D, L792K, P793Q, P793G, Q804A, Y966N, Y723N, Y857R, S890R, S932M, L897M, R624G, S603G, N737S, L307K, I658V \hat{ }PT688, \hat{ }SA794, S877R, N580T, V335G, T620S, W345G, T280S, L406P, A612D, A751S, E386R, V351M, K210N, D40A, E773G, H207L, T62A, T287P, T832A, A893S, \hat{ }V14, \hat{ }AG13, R11V, R12N, R13H, \hat{ }Y13, R12L, \hat{ }Q13,V15S, \hat{ }D17. \hat{ } indicate insertions, [ ] indicate deletions.

FIG. 11 is a plot showing individual beneficial mutations can be combined (sometimes referred to as “stacked”) for even greater improvements in gene editing activity, as described in Example 5. CasX proteins were tested for their ability to cleave at 2 different target sites in human HEK293 cells using the E6 and E7 spacers targeting an EGFP reporter, as described in Example 5. The variants, from left to right, are: S794R+Y797L, K416E+A708K, A708K+[P793], [P793]+P793AS, Q367K+I425S, A708K+[P793]+A793V, Q338R+A339E, Q338R+A339K, S507G+G508R, L379R+A708K+[P793], C477K+A708K+[P793], L379R+C477K+A708K+[P793], L379R+A708K+[P793]+A739V, C477K+A708K+[P793]+A739V, L379R+C477K+A708K+[P793]+A739V, L379R+A708K+[P793]+M779N, L379R+A708K+[P793]+M771N, L379R+A708K+[P793]+D489S, L379R+A708K+[P793]+A739T, L379R+A708K+[P793]+D732N, L379R+A708K+[P793]+G791M, L379R+A708K+[P793]+Y797L, L379R+C477K+A708K+[P793]+M779N, L379R+C477K+A708K+[P793]+M771N, L379R+C477K+A708K+[P793]+D489S, L379R+C477K+A708K+[P793]+A739T, L379R+C477K+A708K+[P793]+D732N, L379R+C477K+A708K+[P793]+G791M, L379R+C477K+A708K+[P793]+Y797L, L379R+C477K+A708K+[P793]+T620P, A708K+[P793]+E386S, E386R+F399L+[P793] and R4581I+A739V of the reference CasX protein of SEQ ID NO: 2. [ ] refer to deleted amino acid residues at the specified position of SEQ ID NO: 2.

FIGS. 12A-12B are a pair of plots showing that CasX protein and sgNA variants when combined, can improve activity more than 6-fold relative to a reference sgRNA and reference CasX protein pair. sgNA:protein pairs were assayed for their ability to cleave a GFP reporter in HEK293 cells, as described in Example 5. On the Y-axis, the fraction of cells in which expression of the GFP reporter was disrupted by CasX mediated gene editing are shown. FIG. 12A shows CasX protein and sgNAs that were assayed with the E6 spacer targeting GFP. FIG. 12B shows CasX protein and sgNAs that were assayed with the E7 spacer targeting GFP. iGFP stands for “inducible GFP.”

FIGS. 13A-13C show that making and screening DME libraries has allowed for generation and identification of variants that exhibit a 1 to 81-fold improvement in editing efficiency, as described in Examples 1 and 3. FIG. 13A shows an RFP+ and GFP+ reporter in E. coli cells assayed for CRISPR interference repression of GFP with a reference nuclease dead CasX protein and sgNA. FIG. 13B shows the same reporter cells assayed for GFP repression with nuclease dead CasX variants screened from a DME library. FIG. 13C shows improved editing efficiency of a selected CasX protein and sgNA variant compared to the reference with 5 spacers targeting the endogenous B2M locus in HEK 293 human cells. The Y axis shows disruption in B2M staining by HLA1 antibody indicating gene disruption via CasX editing and indel formation. The improved CasX variants improved editing of this locus up to 81-fold over the reference in the case of guide spacer #43. CasX pairs with the reference sgRNA: protein pair of SEQ ID NO: 5 and SEQ ID NO: 2; and CasX variant protein of L379R+A708K+[P793] of SEQ ID NO: 2, assayed with the sgNA variant with a truncated stem loop and a T10C substitution, which is encoded by a sequence of TACTGGCGCCTTTATCTCATTACTTTGAGAGCCATCACCAGCGACTATGTCGTATGG GTAAAGCGCTTACGGACTTCGGTCCGTAAGAAGCATCAAAG (SEQ ID 23), are shown. The following spacer sequences were used: #9: GTGTAGTACAAGAGATAGAA (SEQ ID NO: 24); #14: TGAAGCTGACAGCATTCGGG (SEQ ID NO: 25), #20: tagATCGAGACATGTAAGCA (SEQ ID NO: 26); #37: GGCCGAGATGTCTCGCTCCG (SEQ ID NO: 27) and #43: AGGCCAGAAAGAGAGAGTAG (SEQ ID NO: 28).

FIGS. 14A-14F are a series of structural models of a prototypic CasX protein showing the location of mutations in CasX variant proteins of the disclosure which exhibit improved activity, as described in Example 14. FIG. 14A shows a deletion of P at 793 of SEQ ID NO: 2, with a deletion in a loop that may affect folding. FIG. 14B shows a replacement of Alanine (A) by Lysine (K) at position 708 of SEQ ID NO: 2. This mutation is facing the gNA 5′ end plus a salt bridge to the gNA. FIG. 14C shows a replacement of Cysteine (C) by Lysine (K) at position 477 of SEQ ID NO: 2. This mutation is facing the gNA. There is salt bridge to the gNAbb (gNA phosphase backbone) at approximately base 14 that may be affected. This mutation removes a surface exposed cysteine. FIG. 14D shows a replacement of Leucine (L) with Arginine (R) at position 379 of SEQ ID NO: 2. There is a salt bridge to the target DNAbb (DNA phosphate backbone) towards base pairs 22-23 that may be affected. FIG. 14E shows one view of a combination of the deletion of P at 793 and the A708K substitution. FIG. 14F shows an alternate view, that shows that the effects of individual mutants are additive and single mutants can be combined (stacked) for even greater improvements. Arrows indicate the locations of mutations in FIGS. 14E-14F.

FIG. 15 is a plot showing the identification of optimal Planctomycetes CasX PAM and spacers for genes of interest, as described in Example 19. On the Y-axis, percent GFP negative cells, indicating cleavage of a GFP reporter, is shown. On the X-axis, different PAM sequences and spacers: ATC PAM, CTC PAM and TTC PAM. GTC, TTT and CTT PAMs were also tested and showed no activity.

FIG. 16 is a plot showing that improved CasX variants generated by DME edit both canonical and non-canonical PAMs more efficiently than reference CasX proteins, as described in Example 19. The Y-axis shows the average fold improvement in editing relative to a reference sgRNA: protein pair (SEQ ID NO:2, SEQ ID NO: 5) with 2 targets, N=6. Protein variants, from left to right for each set of bars were: A708K+[P793]+A739V; L379R+A708K+[P793]; C477K+A708K+[P793]; L379R+C477K+A708K+[P793]; L379R+A708K+[P793]+A739V; C477K+A708K+[P793]+A739V; and L379R+C477K+A708K+[P793]+A739V. Reference CasX and protein variants were assayed with a reference sgRNA scaffold of SEQ ID NO: 5 with DNA encoding spacer sequences of, from left to right, E6 (TGTGGTCGGGGTAGCGGCTG; SEQ ID NO: 29) with a TTC PAM; E7 (TCAAGTCCGCCATGCCCGAA; SEQ ID NO: 30) with a TTC PAM; GFP8 (CCAGGGTGTCGCCCTCGAAC; SEQ ID NO: 31) with a TTC PAM; B1 (TGACCACCCTGACCTACGGC; SEQ ID NO: 32) with a CTC PAM and A7 (TGGGGCACAAGCTGGAGTAC; SEQ ID NO: 33) with an ATC PAM.

FIGS. 17A-17F are a series of plots showing that a reference CasX protein and a reference sgRNA scaffold pair is highly specific for the target sequence, as described in Example 14. FIG. 17A and FIG. 17D, Streptococcus pyogenes Cas9 (SpyCas9) was assayed with two different gNA spacers and a 5′ PAM site (SEQ ID NOs: 34-65) and (SEQ ID NOs: 136-166) for its ability to edit templates with a target sequence complementary to the spacer sequence (arrow), or with 1, 2, 3 or 4 mutations in the target sequence relative to the spacer sequence. FIG. 17B and FIG. 17E, Staphylococcus aureus Cas9 (SauCas9) was assayed with two different gNA spacers and a 5′ PAM site (SEQ ID NOs: 66-103) and (SEQ ID NOs: 167-204) for its ability to edit templates with a target sequence complementary to the spacer sequence (arrow), or with 1, 2, 3 or 4 mutations in the target sequence relative to the spacer sequence. FIG. 17C and FIG. 17F, the reference Plm CasX protein and sgNA scaffold pair was assayed with two different gNA spacers and a 3′ PAM site (SEQ ID NOs: 104-135) and (SEQ ID NOs: 205-236) for its ability to edit templates with a target sequence complementary to the spacer sequence (arrow), or with 1, 2, 3 or 4 mutations in the target sequence relative to the spacer sequence. In all of FIG. 17A-17F, the X-axis shows the fraction of cells where gene editing at the target sequence occurred.

FIG. 18 illustrates a scaffold stem loop of an exemplary reference sgRNA of the disclosure (SEQ ID NO: 237).

FIG. 19 illustrates an extended stem loop sequence of an exemplary reference sgRNA of the disclosure (SEQ ID NO: 238).

FIGS. 20A-20B are a pair of plots that demonstrate that specific subsets of changes discovered by DME of the CasX are more likely to predict improvements of activity, as described in Example 16. The plots represent data from the experiments described in FIGS. 7A-7I and FIGS. 8A-8C. FIG. 20A shows that changing amino acids within a distance of 10 Angstroms (A) of the guide RNA to hydrophobic residues (A, V, I, L, M, F, Y, W) results in a significantly less active protein. FIG. 20B demonstrates that, in contrast, changing a residue within 10 A of the RNA to a positively charged amino acid (R, H, K) is likely to improve activity.

FIG. 21 illustrates an alignment of two reference CasX protein sequences (SEQ ID NO: 1, top; SEQ ID NO: 2, bottom), with domains annotated.

FIG. 22 illustrates the domain organization of a reference CasX protein of SEQ ID NO: 1. The domains have the following coordinates: non-target strand binding (NTSB) domain: amino acids 101-191; Helical I domain: amino acids 57-100 and 192-332; Helical II domain: 333-509; oligonucleotide binding domain (OBD): amino acids 1-56 and 510-660; RuvC DNA cleavage domain (RuvC): amino acids 551-824 and 935-986; target strand loading (TSL) domain: amino acids 825-934. Not that the Helical I, OBD and RuvC domains are non-contiguous.

FIG. 23 illustrates an alignment of two CasX reference sgRNA scaffolds SEQ ID NO: 5 (top) and SEQ ID NO: 4 (bottom).

FIG. 24 is a graph of the results of an assay for the quantification of active fractions of RNP formed by sgRNA174 and the CasX variants 119 and 457, as described in Example 12. Equimolar amounts of RNP and target were co-incubated and the amount of cleaved target was determined at the indicated timepoints. Mean and standard deviation of three independent replicates are shown for each timepoint. The biphasic fit of the combined replicates is shown. “2” refers to the reference CasX protein of SEQ ID NO: 2.

FIG. 25 is a graph of the results of an assay for quantification of active fractions of RNP formed by CasX2 and reference guide 2, and the modified sgRNA guides 32, 64, and 174, as described in Example 12. Equimolar amounts of RNP and target were co-incubated and the amount of cleaved target was determined at the indicated timepoints. Mean and standard deviation of three independent replicates are shown for each timepoint. The biphasic fit of the combined replicates is shown. “2” refers to reference gRNAs SEQ ID NO: 5, respectively, and the identifying number of modified sgRNAs are indicated in Table 3.

FIG. 26 is a graph of the results of an assay for quantification of cleavage rates of RNP formed by sgRNA174 and the CasX variants 119 and 457, as described in Example 12. Target DNA was incubated with a 20-fold excess of the indicated RNP and the amount of cleaved target was determined at the indicated time points. Mean and standard deviation of three independent replicates are shown for each timepoint. The monophasic fit of the combined replicates is shown.

FIG. 27 is a graph of the results of an assay for quantification of cleavage rates of RNP formed by CasX2 and the sgRNA guide variants 2, 32, 64 and 174, as described in Example 12. Target DNA was incubated with a 20-fold excess of the indicated RNP and the amount of cleaved target was determined at the indicated time points. Mean and standard deviation of three independent replicates are shown for each timepoint. The monophasic fit of the combined replicates is shown.

FIG. 28 is a graph of the results of an assay for quantification of initial velocities of RNP formed by CasX2 and the sgRNA guide variants 2, 32, 64 and 174, as described in Example 12. The first two time-points of the previous cleavage experiment were fit with a linear model to determine the initial cleavage velocity.

FIG. 29 shows the results of an editing assay of 6 target genes in HEK293T cells, as described in Example 15. Each dot represents results using an individual spacer.

FIG. 30 shows the results of an editing assay of 6 target genes in HEK293T cells, with individual bars representing the results obtained with individual spacers, as described in Example 15.

FIG. 31 shows the results of an editing assay of 4 target genes in HEK293T cells, as described in Example 15. Each dot represents results using an individual spacer utilizing a CTC PAM.

FIG. 32 is a schematics showing the steps of Deep Mutational Evolution used to create libraries of genes encoding CasX variants, as described in Example 16. The pSTX1 backbone is minimal, composed of only a high-copy number origin and KanR resistance gene, making it compatible with the recombineering E. coli strain EcNR2. pSTX2 is a BsmbI destination plasmid for aTc-inducible expression in E. coli.

FIG. 33 are dot plot graphs showing the results of CRISPRi screens for mutations in libraries D1, D2, and D3, as described in Example 16. In the absence of CRISPRi, E. coli constitutively express both GFP and RFP, resulting in intense fluorescence in both wavelengths, represented by dots in the upper-right region of the plot. CasX proteins resulting in CRISPRi of GFP can reduce green fluorescence by >10-fold, while leaving red fluorescence unaltered, and these cells fall within the indicated Sort Gate 1. The total fraction of cells exhibiting CRISPRi is indicated.

FIG. 34 are photographs of colonies grown in the ccdB assay, as described in Example 16. 10-fold dilutions were assayed in the presence of glucose or arabinose to induce expression of the ccdB toxin, resulting in approximately a 1000-fold difference between functional and nonfunctional proteins. When grown in liquid culture, the resolving power was approximately 10,000-fold, as seen on the right-hand side.

FIG. 35 is a graph of HEK iGFP genome editing efficiency testing CasX variants with sgRNA 2 (SEQ ID NO: 5), with appropriate spacers, with data expressed as fold-improvement over the wild-type CasX protein (SEQ ID NO: 2) in the HEK iGFP editing assay, as described in Example 16. Single mutations are shown at the top, with groups of mutations shown at the bottom of the graph. Error bars combine internal measurement error (SD) and inter-experimental measurement error (SD across replicate experiments for those variants tested more than once), in at least triplicate assays.

FIG. 36 is a scatterplot showing results of the SOD1-GFP reporter assay for CasX variants with sgRNA scaffold 2 utilizing two different spacers for GFP, as described in Example 16.

FIG. 37 is a graph showing the results of the HEK293 iGFP genome editing assay assessing editing across four different PAM sequences comparing wild-type CasX (SEQ ID NO:2) and CasX variant 119; both utilizing sgRNA scaffold 1 (SEQ ID NO:4), with spacers utilizing four different PAM sequences, as described in Example 16.

FIG. 38 is a graph showing the results of genome editing activity of CasX variant 119 and sgRNA 174 compared to wild-type CasX 2 and guide scaffold 1 in the iGFP lipofection assay utilizing two different spacers, as described in Example 16.

FIG. 39 is a graph showing the results of genome editing activity of CasX variant 119 and sgRNA 174 compared to wild-type CasX and guide in the iGFP lentiviral transduction assay, as described in Example 16.

FIG. 40 is a graph showing the results of genome editing in the more stringent lentiviral assay to compare the editing activity of four CasX variants (119, 438, 488 and 491) and the optimized sgNA 174 and two different spacers, as described in Example 16. The results show the step-wise improvement in editing efficiency achieved by the additional modifications and domain swaps introduced to the starting-point 119 variant.

FIGS. 41A-41B show the results of NGS analyses of the libraries of sgRNA, as described in Example 17. FIG. 41A shows the distribution of substitutions, deletions and insertions. FIG. 41B is a scatterplot showing the high reproducibility of variant representation in two separate library pools after the CRISPRi assay in the unsorted, naive population of cells. (Library pool D3 vs D2 are two different versions of the dCasX protein, and represent replicates of the CRISPRi assay.)

FIGS. 42A-42B shows the structure of wild-type CasX and RNA guide (SEQ ID NO:4). FIG. 42A depicts the CryoEM structure of Deltaproteobacteria CasX protein:sgRNA RNP complex (PDB id: 6YN2), including two stem loops, a pseudoknot, and a triplex. FIG. 42B depicts the secondary structure of the sgRNA was identified from the structure shown in (A) using the tool RNAPDBee 2.0 (rnapdbee.cs.put.poznan.pl/, using the tools 3DNA/DSSR, and using the VARNA visualization tool). RNA regions are indicated. Residues that were not evident in the PDB crystal structure file are indicated by plain-text letters (i.e., not encircled), and are not included in residue numbering.

FIGS. 43A-43C depicts comparisons between two guide RNA scaffolds. FIG. 43A provides the sequence alignment between the single guide scaffold 1 (SEQ ID NO:4) and scaffold 2 (SEQ ID NO:5). FIG. 43B shows the predicted secondary structure of scaffold 1 (without the 5′ ACAUCU bases which were not in the cryoEM structure). Prediction was done using RNAfold (v 2.1.7), using a constraint that was derived from the base-pairing observed in the cryoEM structure (see FIG. 42A-42B). This constraint required the base pairs observed in the cryoEM structure to be formed, and required the bases involved in triplex formation to be unpaired. This structure has distinct base pairing from the lowest-energy predicted structure at the 5′ end (i.e., the pseudoknot and triplex loop). FIG. 43C shows the predicted secondary structure of scaffold 2. Prediction was done for scaffold 1, using a similar constraint based on the sequence alignment.

FIG. 44 shows a graph comparing GFP-knockdown capability of scaffold 1 versus scaffold 2 in GFP-lipofection assay, using four different spacers utilizing different PAM sequences, as described in Example 17. The results demonstrate the greater editing imparted by use of the modified scaffold 2 compared to the wild-type scaffold 1; the latter showing no editing with spacers utilizing GTC and CTC PAM sequences.

FIGS. 45A-45C show graphs depicting the enrichment of single variants across the scaffold, revealing mutable regions, as described in Example 17. FIG. 45A depicts substituted bases (A, T, G, or C; top to bottom), FIG. 45B depicts inserted bases (A, T, G, or C; top to bottom), and FIG. 45C depicts deletions at the individual nucleotide position (X-axis) across scaffold 2. Enrichment values were averaged across the three deadCasX versions, relative to the average WT value. Scaffolds with relative log2 enrichment >0 are considered ‘enriched’, as they were more represented in the sorted population relative to the naive population than the wildtype scaffold was represented. Error bars represent the confidence interval across the three catalytically dead CasX experiments.

FIG. 46 are scatterplots showing that the enrichment values obtained across different dCasX variants are largely consistent, as described in Example 17. Libraries D2 and DDD have highly correlated enrichment scores, while D3 is more distinct.

FIG. 47 shows a bar graph of cleavage activity of several scaffold variants in a more stringent lipofection assay at the SOD1-GFP locus, as described in Example 17.

FIG. 48 shows a bar graph of cleavage activity for several scaffold variants using two different spacers; 8.2 and 8.4 that target SOD1-GFP locus (and a non-targeting spacer NT), with low-MOI lentiviral transduction using a p34 plasmid backbone, as described in Example 15.

FIG. 49 is a schematic showing the secondary structure of single guide 174 on top and the linear structure on the bottom, with lines joining those segments associating by base-pairing or other non-covalent interactions. The scaffold stem (white, no fill) (and loop) and the extended stem (grey, no fill) (and loop) are adjacent from 5′ to 3′ in the sequence. However, the pseudoknot and extended stems are formed from strands that have intervening regions in the sequence. The triplex is formed, in the case of single guide 174, comprising nucleotides 5′-CUUUG′-3′ AND 5′-CAAAG-3′ that form a base-paired duplex and nucleotides 5′-UUU-3′ that associates with the 5′-AAA-3′ to form the triplex region.

FIGS. 50A-50B shows comparisons between the highly-evolved single guide 174 and the scaffolds 1 and 2 that served as the starting points for the DME procedures described in Example 17. FIG. 50A shows a bar graph of cleavage activity of head-to-head comparisons of cleavage activity of the guide scaffolds with five different spacers in a plasmid lipofection assay at the GFP locus in HEK-GFP cells. FIG. 50B shows the sequence alignment between scaffold 2 and guide 174 (SEQ ID NO: 2238). Asterisks indicate point mutations, and the dotted box shows the entire extended stem swap.

FIGS. 51A-51B shows scatterplots of HEK-iGFP cleavage assay for scaffolds sequences relative to WT scaffold with 2 spacers; 4.76 (FIG. 51A) and 4.77 (FIG. 51B), as described in Example 17.

FIG. 52 shows a scatterplot comparing the normalized cleavage activity of several scaffolds relative to WT with 2 spacers (4.76 and 4.77), as described in Example 17. Error bars combine internal measurement error (SD) and inter-experimental measurement error (SD across replicate experiments for those variants tested more than once), in quadrature.

FIG. 53 shows a scatterplot comparing the normalized cleavage activity of multiple scaffolds relative to WT in the HEK-iGFP cleavage assay to the enrichments obtained from the CRISPRi comprehensive screen, as described in Example 17. Generally, scaffold mutations with high enrichment (>1.5) have cleavage activity comparable to or greater than WT. Two variants have high cleavage activity with low enrichment scores (C18G and T17G); interestingly, these substitutions are at the same position as several highly enriched insertions (FIGS. 45A-45C). Labels indicate the mutations for a subset of the comparisons.

### DETAILED DESCRIPTION

While exemplary embodiments have been shown and described herein, it will be obvious to those skilled in the art that such embodiments are provided by way of example only. Numerous variations, changes, and substitutions will now occur to those skilled in the art without departing from the inventions claimed herein. It should be understood that various alternatives to the embodiments described herein may be employed in practicing the embodiments of the disclosure. It is intended that the claims define the scope of the invention and that methods and structures within the scope of these claims and their equivalents be covered thereby.

All publications, patents, and patent applications mentioned in this specification are herein incorporated by reference to the same extent as if each individual publication, patent, or patent application was specifically and individually indicated to be incorporated by reference.

### I. General Methods

The practice of the present invention employs, unless otherwise indicated, conventional techniques of immunology, biochemistry, chemistry, molecular biology, microbiology, cell biology, genomics and recombinant DNA, which can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (1. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that endpoints are included and that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

It will be appreciated that certain features of the disclosure, which are, for clarity, described in the context of separate embodiments, may also be provided in combination in a single embodiment. In other cases, various features of the disclosure, which are, for brevity, described in the context of a single embodiment, may also be provided separately or in any suitable sub-combination. It is intended that all combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### II. DME Methods for Generation of Improved Gene Editing Molecules

Provided herein are methods of generating and selecting improved biomolecule variants, such as RNA, DNA, or protein variants, through Deep Mutational Evolution (DME). Also provided are the biomolecule variants selected from said methods, and libraries of variants which may be used in said methods.

In some embodiments, the methods, variants, and libraries described herein may include insertions and/or deletions, in addition to substitution mutations. In some embodiments, the DME methods provided herein include constructing and screening one or more libraries representing a comprehensive set of mutations of a biomolecule, e.g. encompassing all possible substitutions, as well as insertions and deletions of one or more amino acids (in the case of proteins), or one or more ribonucleotides (in the case of RNA), or one or more deoxyribonucleotides (in the case of DNA). In other embodiments, a subset of such mutations is screened. In some embodiments, screening of one or more libraries of biomolecule variants is used to obtain information about how certain mutations (such as insertion and/or deletion and/or substitution, or combinations thereof) or the mutation to certain regions of a reference biomolecule affects the functional properties of said biomolecule, or affect the functional properties of a protein encoded by said biomolecule. In some embodiments, modifications resulting in one or more improved characteristics are then combined in one or more additional rounds of biomolecule modification, either through rational design or randomly, and these second round variants are screened to identify desirable characteristics. Additional libraries may be constructed and screened using information obtained from the previous library, and through such iterative processes, in some embodiments, one or more biomolecule variants are selected. Thus, for example, in some embodiments the methods provided herein comprise a second, third, fourth, fifth, or more rounds of variant construction and screening. In certain embodiments, such biomolecule variants may have one or more improved characteristics, which are described in greater detail herein. In still other embodiments, such biomolecule variants may encode for a protein with one or more improved characteristics, which are described in greater detail herein. Such iterative construction and evaluation of variants may lead, for example, to identification of mutational themes that lead to certain functional outcomes, such as identification of types of mutations or of regions of the protein or RNA that when mutated in a certain way lead to one or more improved or altered functions. Layering of such identified mutations may then further improve function, for example through additive or synergistic interactions. The use of iterative rounds of biomolecule evolution may progressively improve/alter one or more functional characteristics of the variant biomolecules, resulting in a highly functional protein, RNA, or DNA variant that is specialized for a desired application.

In some embodiments, these methods include constructing a library comprising a plurality of variants of a reference biomolecule, wherein each variant independently has an alteration of at least one monomer location (e.g., ribonucleotide for RNA, or amino acid for protein, or deoxyribonucleotide for DNA), and wherein the alterations can independently include insertion of one or more monomers, deletion of one or more monomers, or substitution of the monomer. In some embodiments, the library collectively represents alteration of at least 1%, or at least 10%, or up to 100%, of the monomer locations of the reference biomolecule. This may include, for example, libraries wherein each variant only has one alteration of one monomer location, but collectively the library represents alteration of at least 1%, or at least 10%, or up to 100%, of the monomer locations of the reference biomolecule. In certain embodiments, the library collectively represents each possible alteration of at least 1%, or at least 10%, or up to 100%, of the monomer locations of the reference biomolecule.

I. Libraries

Provided herein are methods and systems for developing variants of biomolecules, such as proteins, RNA, and DNA, that include evaluating insertions and deletions of monomers in addition to substitutions. Such methods include constructing one or more libraries of variants of a reference biomolecule, and evaluating said libraries for change in one or more characteristics of the variants compared to the reference biomolecule. Such information can be used, for example to construct one or more additional variants and/or libraries, such as by layering mutations with a desired effect on certain characteristics, or by selecting a subset of the initial library and subjecting it to a round of random mutation, or by taking information learned from screening of a library and using it to construct a new variant with additional alterations. In some embodiments, an iterative process of library construction, evaluation, and new library construction is used.

Proteins, RNA, and DNA are polymers composed of amino acid, ribonucleotide, and deoxyribonucleotide monomers, respectively. For each monomer location, there are three types of variations possible: l) substitution of the original monomer for another monomer; 2) insertion of one or more consecutive monomers; and 3) deletion of one or more consecutive monomers. DME libraries comprising substitutions, insertions, and deletions, alone or in combination, to any one or more monomers within any biomolecule described herein, are considered within the scope of the invention.

The complexity of variations is further increased when taking into account the number of different monomers that can be used in substitution or each single insertion—20 different naturally occurring amino acids for proteins, and 4 naturally occurring nucleotides for RNA and DNA. Therefore, with respect to naturally occurring amino acids and naturally occurring ribonucleotides, the number of possible alterations per monomer location for a protein includes: 19 possible monomer (amino acid) substitutions, 20 possible monomer insertions (per single insertion), 1 possible monomer deletion (per single deletion). The number of possible alterations per monomer location for RNA or DNA includes: 3 possible monomer (nucleotide) substitutions, 4 possible monomer insertions (per single insertion), 1 possible monomer deletion (per single deletion).

A library used in the methods described herein may, in some embodiments, comprise substitutions, insertions, and deletions, alone or in combination, to one or more monomers within any biomolecule described herein. In some embodiments of the methods, every possible single alteration of every monomer is evaluated. For example, in some embodiments one or more libraries of variants are constructed and evaluated, wherein each variant independently comprises a single alteration compared to the reference biomolecule, and the one or more libraries collectively represent every possible single alteration of every monomer location. In some embodiments, insertion of two or more monomers at every monomer location is evaluated, or deletion of two or more monomers at very monomer location is evaluated, or a combination thereof. For example, for a reference protein of 1000 residues, there are 1000 possible single amino acid deletions, 1.9*10\hat{ }4 possible amino acid substitutions, and 2*10\hat{ }4 possible single amino acid insertions. For double amino acid insertions, there are 4*10\hat{ }5 possible variants; likewise, triples have 8*10\hat{ }6 variants and so forth. In some embodiments, one or more libraries are built to evaluate the comprehensive set of mutations to a biomolecule, encompassing all possible substitutions, as well as insertions and deletions of, for example, between 1 to 4 amino acids (in the case of proteins) or nucleotides (in the case of RNA or DNA). In some embodiments, one or more libraries are built to evaluate a subset of a comprehensive set of mutations to a biomolecule, encompassing all possible substitutions to a particular region of a biomolecule, as well as insertions and deletions to a particular region of a biomolecule of, for example, between 1 to 4 amino acids (in the case of proteins) or nucleotides (in the case of RNA or DNA).

In some embodiments, the library comprises a subset of all possible alterations to monomers. For example, in some embodiments, a library collectively represents a single alteration of one monomer, for at least 1%, or at least 10% of the total monomer locations in a biomolecule, wherein each single alteration is selected from the group consisting of substitution, single insertion, and single deletion. In some embodiments, the library collectively represents the single alteration of one monomer, for at least 20%, at least 30%, at least 40%, at least 50%, at least 60%, at least 70%, at least 80%, at least 90%, at least 95%, or up to 100% of the total monomer locations in a starting biomolecule (e.g., each variant comprises one modified monomer, and the collection of variants represent single alteration of one monomer for at least a certain percentage of total locations). In certain embodiments, for a certain percentage of the total monomer locations in a starting biomolecule, the library collectively represents each possible single alteration of one monomer, such as all possible substitutions with the 19 other naturally occurring amino acids (for a protein) or 3 other naturally occurring ribonucleotides (for RNA) or 3 other naturally occurring deoxyribonucleotides (for DNA), insertion of each of the 20 naturally occurring amino acids (for a protein) or 4 naturally occurring ribonucleotides (for RNA) or 4 naturally occurring deoxyribonucleotides (for DNA), or deletion of the monomer. In still further embodiments, insertion at each location is independently greater than one monomer, for example insertion of two or more, three or more, or four or more monomers, or insertion of between one to four, between two to four, or between one to three monomers. In some embodiments, deletion at each location is independently greater than one monomer, for example deletion of two or more, three or more, or four or more monomers, or deletion of between one to four, between two to four, or between one to three monomers. Examples of such libraries of CasX variants and gNA variants are described in Examples 14 and 15, respectively.

In some embodiments of the methods and compositions provided herein, the monomers used in substitution and/or insertion are naturally occurring monomers (e.g., the 20 naturally occurring standard amino acids; the 4 ribonucleotides A, U, C, and G; and the 4 deoxyribonucleotides A, T, C, and G). In other embodiments, one or more unnatural monomers is used. Such monomers may include, for example, chemically- or enzymatically-modified monomers, chemically synthesized monomers, monomers obtained commercially, or others. In some embodiments, one or more naturally occurring monomers is modified after being incorporated into a variant. For example, in some embodiments, a protein variant is constructed and then one or more amino acid residues of the protein variant are chemically or enzymatically modified to produce the protein variant to be screened. In other embodiments, an unnatural monomer is incorporated into the variant as-is. For example, in certain embodiments one or more RNA or DNA variants are constructed using unnatural nucleotides, which may be obtained commercially or synthesized through techniques known to one of skill in the art.

In some embodiments, the biomolecule is a protein and the individual monomers are amino acids. In those embodiments where the biomolecule is a protein, the number of possible mutations at each monomer (amino acid) position in the protein comprises 19 naturally occurring amino acid substitutions, 20 naturally occurring amino acid insertions and 1 amino acid deletion, leading to a total of 40 possible mutations per amino acid in the protein. In some embodiments, one or more variants comprises substitution of more than one amino acid monomers, wherein each monomer location is independently selected. Thus, for example, in some embodiments a library comprises one or more variants wherein two or more consecutive amino acids are independently substituted. In some embodiments, wherein the library comprises variants independently comprising one or more substitutions, each substitution is a conservative substitution. A conservative substitution replaces the original amino acid with an amino acid that has a similar characteristic. For example, if the original amino acid is glycine, a conservative substitution may be one that replaces the glycine with another aliphatic amino acid, such as alanine, valine, leucine, or isoleucine. If the amino acid is phenylalanine, a conservative substitution may be one that replaces the phenylalanine with another aromatic amino acid, such as tyrosine or tryptophan. In other embodiments of, wherein the library comprises variants independently comprising one or more substitutions, each substitution is a non-conservative substitution (e.g., a substitution with an amino acid that has a different characteristic). In some embodiments, conservative substitution of an amino acid may cause the variant to retain one or more desirable characteristics at that location (e.g., polarity, or charge, or hydrophobic interactions, or another characteristic) while still providing the variability that may lead to one or more improved characteristics of the variant overall. For example, a non-conservative substitution of the original amino acid glycine may be with a charged amino acid, or an aromatic amino acid, or a cyclic amino acid. In still further embodiments, wherein the library comprises variants independently comprising one or more substitutions, each substitution is independently a non-conservative substitution or a conservative substitution.

In other embodiments, the biomolecule is RNA and the individual monomers are ribonucleotides. In those embodiments where the biomolecule is RNA, the number of possible mutations at each monomer (ribonucleotide) position in the RNA comprises 3 naturally occurring ribonucleotide substitutions, 4 naturally occurring ribonucleotide insertions, and 1 naturally occurring ribonucleotide deletion, leading to a total of 8 possible mutations per ribonucleotide in the RNA. In some embodiments, one or more variants comprises substitution of more than one ribonucleotide monomers, wherein each monomer location is independently selected. Thus, for example, in some embodiments a library comprises one or more variants wherein two or more consecutive ribonucleotides are independently substituted.

In still further embodiments, the biomolecule is DNA and the individual monomers are deoxyribonucleotides. In those embodiments where the biomolecule is DNA, the number of possible mutations at each monomer (deoxyribonucleotide) position in the DNA comprises 3 naturally occurring deoxyribonucleotide substitutions, 4 naturally occurring deoxyribonucleotide insertions, and 1 naturally occurring deoxyribonucleotide deletion, leading to a total of 8 possible mutations per deoxyribonucleotide in the DNA. In some embodiments, one or more variants comprises substitution of more than one deoxyribonucleotide monomers, wherein each monomer location is independently selected. Thus, for example, in some embodiments a library comprises one or more variants wherein two or more consecutive deoxyribonucleotides are independently substituted.

In some embodiments, a library of protein variants comprising insertions is a 1 amino acid insertion library, a 2 amino acid insertion library, a 3 amino acid insertion library, a 4 amino acid insertion library, a 5 amino acid insertion library, a 6 amino acid insertion library, a 7 amino acid insertion library, or an 8 amino acid insertion library. In some embodiments, a protein variant library comprises insertions wherein each insertion comprises between 1 and 8 amino acids, between 1 and 7 amino acids, between 1 and 6 amino acids, between 1 and 5 amino acids, between 1 and 4 amino acids, between 1 and 3 amino acids, or 1 or 2 amino acids. In certain embodiments, the library represents insertion of, for example, independently between 1 to 4 amino acids (or 5, or 6, or more) for at least a subset of total monomer locations, such as at least 1%, at least 5%, at least 10%, at least 20%, at least 30%, at least 40%, or up to 90%, or up to 100%. In some embodiments, for each inserted amino acid, the library collectively represents insertion of each of the 20 naturally occurring amino acids at that location. In certain embodiments, for each inserted amino acid, the library collectively represents insertion of at least 1 (e.g., proline scanning), at least 2 (e.g., negative charge scanning), at least 5, at least 10, or at least 15 of the 20 naturally occurring amino acids at that location. Thus, for example, in some embodiments libraries representing the full scope of possible naturally occurring insertions (including variability in the amino acid) for each insertion location are evaluated.

In some embodiments, a library of RNA or DNA variants comprising insertions is a 1 nucleotide insertion library, a 2 nucleotide insertion library, a 3 nucleotide insertion library, a 4 nucleotide insertion library, a 5 nucleotide insertion library, a 6 nucleotide insertion library, a 7 nucleotide insertion library, an 8 nucleotide insertion library, a 9 nucleotide insertion library, a 10 nucleotide insertion library, a 11 nucleotide insertion library, a 12 nucleotide insertion library, a 13 nucleotide insertion library, a 14 nucleotide insertion library, a 15 nucleotide insertion library, a 16 nucleotide insertion library, or more. In some embodiments, an RNA or DNA variant library comprises insertions, wherein each insertion is independently between 1 and 16 nucleotides, between 1 and 14 nucleotides, between 1 and 12 nucleotides, 1 and 10 nucleotides, between 1 and 8 nucleotides, between 1 and 6 nucleotides, between 1 and 4 nucleotides, or 1 or 2 nucleotides. In certain embodiments, the library represents insertion of, for example, independently between 1 to 4 nucleotides (or 5, or 6, or 7, or 8, or up to 16) for at least a subset of total monomer locations, such as at least 1%, at least 5%, at least 10%, at least 20%, at least 30%, at least 40%, or up to 90%, or up to 100%. In some embodiments, for each inserted nucleotide, the library collectively represents insertion of each of the 4 naturally occurring nucleotides at that location (e.g., the four naturally occurring ribonucleotides for RNA, or the four naturally occurring deoxyribonucleotides for DNA). In certain embodiments, for each inserted nucleotide, the library collectively represents insertion of at least 1, at least 2, at least 3, or each of 4 naturally occurring nucleotides at that location. Thus, for example, in some embodiments libraries representing the full scope of possible insertions (including variability in the nucleotide) for each insertion location are evaluated.

In some embodiments, a library of protein variants comprising deletions is a 1 amino acid deletion library, a 2 amino acid deletion library, a 3 amino acid deletion library, a 4 amino acid deletion library, a 5 amino acid deletion library, a 6 amino acid deletion library, a 7 amino acid deletion library, or an 8 amino acid deletion library. In some embodiments, a protein variant library comprises deletions wherein each deletion is independently between 1 and 8 amino acids, between 1 and 7 amino acids, between 1 and 6 amino acids, between 1 and 5 amino acids, between 1 and 4 amino acids, between 1 and 3 amino acids, or 1 or 2 amino acids. In certain embodiments, the library represents deletions of, for example, independently between 1 to 4 amino acids (or 5, or 6, or more) for at least a subset of total monomer locations, such as at least 1%, at least 5%, at least 10%, at least 20%, at least 30%, at least 40%, or up to 90%, or up to 100%.

In some embodiments, a library of RNA or DNA variants comprising deletions is a 1 nucleotide deletion library, a 2 nucleotide deletion library, a 3 nucleotide deletion library, a 4 nucleotide deletion library, a 5 nucleotide deletion library, a 6 nucleotide deletion library, a 7 nucleotide deletions library, an 8 nucleotide deletion library, a 9 nucleotide deletion library, a 10 nucleotide deletion library, a 11 nucleotide deletion library, a 12 nucleotide deletion library, a 13 nucleotide deletion library, a 14 nucleotide deletion library, a 15 nucleotide deletion library, or a 16 nucleotide deletion library. In some embodiments, an RNA or DNA variant library comprises deletions wherein each deletion is independently between 1 and 16 nucleotides, between 1 and 14 nucleotides, between 1 and 12 nucleotides, between 1 and 10 nucleotides, between 1 and 8 nucleotides, between 1 and 6 nucleotides, between 1 and 4 nucleotides, or 1 or 2 nucleotides. In certain embodiments, the library represents deletions of, for example, independently between 1 to 4 nucleotides (or 5, or 6, or more) for at least a subset of total monomer locations, such as at least 1%, at least 5%, at least 10%, at least 20%, at least 30%, at least 40%, or up to 90%, or up to 100%. In some embodiments, wherein the variants are RNA, the nucleotides are ribonucleotides. In other embodiments, wherein the variants are DNA, the nucleotides are deoxyribonucleotides.

In some embodiments, a library of protein variants comprising substitution of at least 1%, at least 5%, at least 10%, at least 20%, at least 30%, at least 40%, or up to 90%, or up to 100% of total monomer locations is evaluated. Such libraries may, in some embodiments, further comprise evaluation of variability in the amino acid used for each insertion location. In some embodiments, for each substituted amino acid, the library collectively represents substitution with each of the other 19 naturally occurring amino acids at that location. In certain embodiments, for each substituted amino acid, the library collectively represents substitution with at least 5, at least 10, or at least 15 of the other 19 naturally occurring amino acids at that location.

In some embodiments, a library of RNA or DNA variants comprising substitution of at least 1%, at least 5%, at least 10%, at least 20%, at least 30%, at least 40%, or up to 90%, or up to 100% of total monomer locations is evaluated. Such libraries may, in some embodiments, further comprise evaluation of variability in the nucleotide used for each insertion location. In some embodiments, for each substituted nucleotide, the library collectively represents substitution with each of the other 3 naturally occurring nucleotides at that location. In certain embodiments, for each substituted nucleotide, the library collectively represents substitution with at least 1, at least 2, or each of the 3 other naturally occurring nucleotides at that location.

It should be further understood that libraries used in the methods described herein may comprise combinations of insertions, substitutions, and deletions, as described herein. Thus, a library representing each possible alteration of at least 1%, at least 5%, at least 10%, at least 20%, at least 30%, at least 40%, at least 50%, or up to 70%, or up to 80%, or up to 90%, or up to 100% of individual monomer locations is, in some embodiments, evaluated. Furthermore, in some embodiments, alterations are layered, such that a single variant may comprise an insertion and a deletion, an insertion and a substitution, a deletion and a substitution, or each of an insertion, a deletion, and a substitution, at different locations of the biomolecule. In certain embodiments, each variant independently comprises between one to sixteen, one to fourteen, one to twelve, one to ten, one to eight, one to six, between one to five, between one to four, between one to three, between one to two, at least one, at least two, at least three, at least four, at least five, or at least six alterations independently selected from the group consisting of substitution, insertion, and deletion.

Thus, in some embodiments, the library comprises variants each independently comprising alteration of one or more locations, wherein collectively the library represents alteration of at least 1%, at least 5%, at least 10%, at least 30%, at least 50%, at least 80%, or at least 99% of the total locations of the reference molecule. In certain embodiments, the library comprises variants each independently comprising alteration of two or more locations, three or more locations, four or more locations, between one and ten locations, between one and eight locations, between one and six locations, or between one and four locations; wherein collectively the library represents alteration of at least 1%, at least 5%, at least 10%, at least 30%, at least 50%, at least 80%, or at least 99% of the total locations of the reference molecule.

In some embodiments, a reference biomolecule can have at least 1, 2, 3, 4, 5, 6, 7, 8, 9, 10, 20, 30, 40, 50, 100 or more monomers that are systematically mutated to produce a library of biomolecule variants. In some embodiments, every monomer in a biomolecule is varied independently. For example, wherein the biomolecule is a protein with two target amino acids, a library design may enumerate the 40 possible mutations at each of the two target amino acids.

In some embodiments, each varied monomer of a biomolecule is independently randomly selected; in other embodiments, each varied monomer of a biomolecule is selected by intentional design, or by previous random mutations that had desired characteristics. Thus, in some embodiments, a library comprises random variants, variants that were designed, variants comprising random mutations and designed mutations within a single biomolecule, or any combinations thereof.

Further provided herein are methods of selecting an improved biomolecule using one or more libraries as described herein. For example, in some embodiments, provided herein is a method of selecting an improved biomolecule variant, wherein the biomolecule is a protein or RNA, the method comprising:
(i) constructing a library of biomolecule variants as described herein, wherein each variant is independently a variant of the same reference biomolecule;
(ii) screening the library of (i);
(iii) identifying at least a portion of the library of (i) that exhibits one or more improved characteristics compared to the reference biomolecule; and
(iv) selecting the improved biomolecule variant from the identified at least a portion of the library, wherein the improved biomolecule variant exhibits one or more improved characteristics compared to the reference biomolecule.

In some embodiments, the library of biomolecule variants of (i) comprises a plurality of biomolecule variants:
wherein each variant is independently a variant of the same reference biomolecule, wherein each variant comprises an alteration of one or more monomer locations of the reference biomolecule, wherein the monomer is an amino acid of the protein or ribonucleotide of the RNA, and
wherein each alteration of a monomer location is independently selected from the group consisting of substitution of the monomer, deletion of one or more consecutive monomers beginning at the location, and insertion of one or more consecutive monomers adjacent to the location;
wherein the library represents variants comprising alteration of one or more locations for at least 1% of the monomer locations of the reference biomolecule.

It should be understood that any library as has been described herein may be used in the methods provided herein. For example, in some embodiments the library represents variations comprising alteration of one or more locations for at least 1%, at least 5%, at least 10%, at least 20%, at least 30%, at least 40%, at least 50%, at least 60%, at least 70%, at least 80%, at least 90%, at least 95%, or up to 100% of the monomer locations of the reference biomolecule. In certain embodiments the library comprises variants in which each variant has one or more, two or more, three or more, or greater than three alterations, or has at least two different types of alterations, or has only one type of alteration, or any combinations that have been described herein.

In some embodiments, the library comprises biomolecule variants with a single alteration of four monomer locations. In certain embodiments, the library comprises variants representing a single alteration of a single location for at least 1% of the total monomer locations, at least 10% of the total monomer locations, at least 30% of the total monomer locations, at least 70% of the total monomer locations, or at least 90% of the total monomer locations. In some embodiments, the library comprises variants representing deletion of one or more monomers beginning at the location, and variants comprising insertion of one or more new monomers adjacent to the location, for at least 30% of monomer locations. In still further embodiments, the library comprises variants representing insertion of each of one, two, three, and four monomers adjacent to the location for at least 80% of the monomer locations. In some embodiments, for each inserted new monomer, the library represents each naturally occurring monomer possibility (e.g., 20 naturally occurring amino acids, or 4 naturally occurring nucleotides). In some embodiments, wherein the library comprises variants with one or more insertions adjacent to a monomer location, each insertion is independently upstream or downstream of the monomer location. In other embodiments, each insertion is downstream of the location (e.g., in some libraries, insertion adjacent to a specified monomer location always indicates the insertion is downstream of that location). In still further embodiments, each insertion is upstream of the location. In some embodiments, deletion of one or more consecutive monomers comprises deletion of between one to four consecutive monomers. In certain embodiments, the library comprises variants representing deletion of each of one, two, three, and four consecutive monomers for at least 80% of the monomer locations. In some embodiments, the substitution of the monomer comprises replacing the monomer with one of the other naturally occurring monomers (e.g., 19 other naturally occurring amino acids, or 3 other naturally occurring nucleotides). In some embodiments, wherein the biomolecule is protein, the library comprises variants that collectively represent in which the same monomer is replaced with each of ten other naturally occurring amino acids, or each of the nineteen other naturally occurring amino acids. In other embodiments, wherein the biomolecule is RNA, library comprises variants that collectively represent in which the same monomer is replaced with each of the three other naturally occurring ribonucleotides. In still further embodiments, wherein the biomolecule is DNA, library comprises variants that collectively represent in which the same monomer is replaced with each of the three other naturally occurring deoxyribonucleotides.

In still further embodiments, the library comprises variants for each of following alterations for at least 80% of the monomer locations:
deletion of each of one, two, three, and four consecutive monomers,
insertion of each of one, two three, and four consecutive monomers, and
substitution of the same monomer with each of the other naturally occurring monomers.

In some embodiments of said library, each variant independently comprises one, two, three, four, five, six, seven, eight, nine, ten, eleven, twelve, thirteen, fourteen, fifteen, sixteen, seventeen, eighteen, nineteen, twenty, or greater alterations itself, and the library as a collective represents the described alterations for at least 80% of the total monomer locations of the reference biomolecule.

In yet further embodiments, provided herein are methods of using the information gained from screening one or more libraries as provided herein to construct one or more additional variants, or libraries. Screening a library may provide information about what types and locations of alterations have a positive, negative, or neutral effect on one or more characteristics of a reference biomolecule. Such information may be used in the construction of one or more additional variants, or in one or more additional libraries. While a variant with a particular improved characteristic may be desired, information regarding what alterations have a neutral or negative effect can also be helpful. For example, screening variants may demonstrate that varying a particular region of a reference biomolecule has little effect on desired characteristics, indicating this region is highly mutable with few negative results and therefore may, without wishing to be bound by any theory, be a flexible region to alter for different purposes. This information could be useful, for example, to inform the location of a handle or tag for a future variant, or to alter the sequence for improved expression or to adapt to a new expression system.

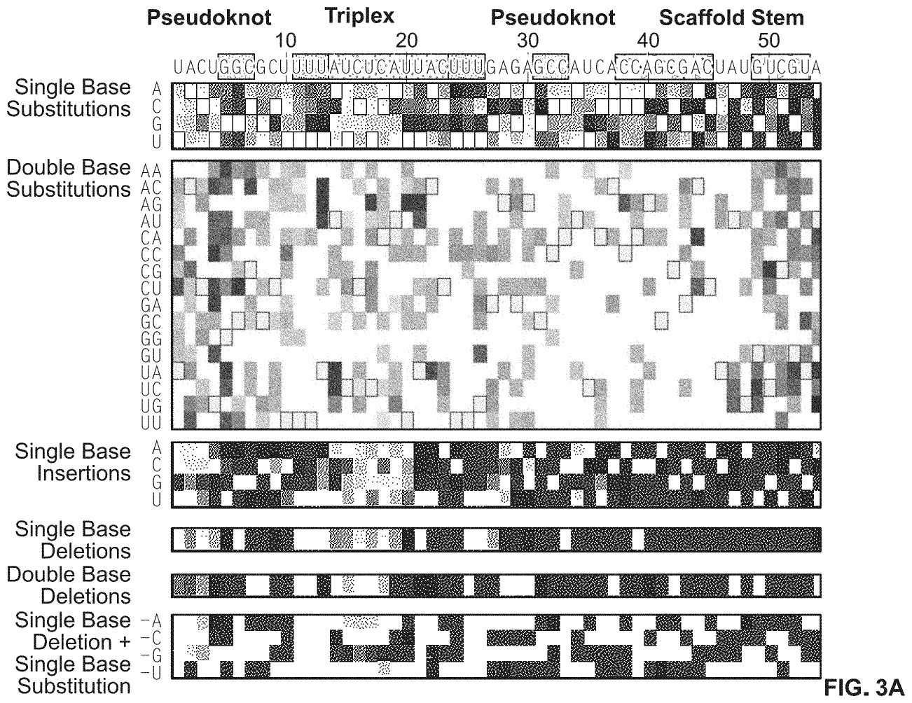

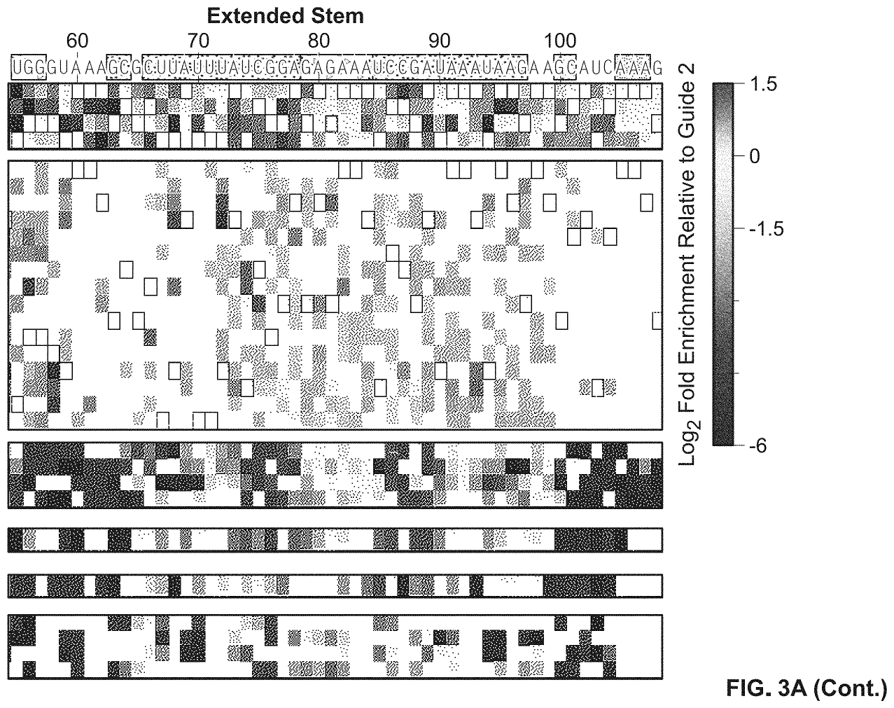

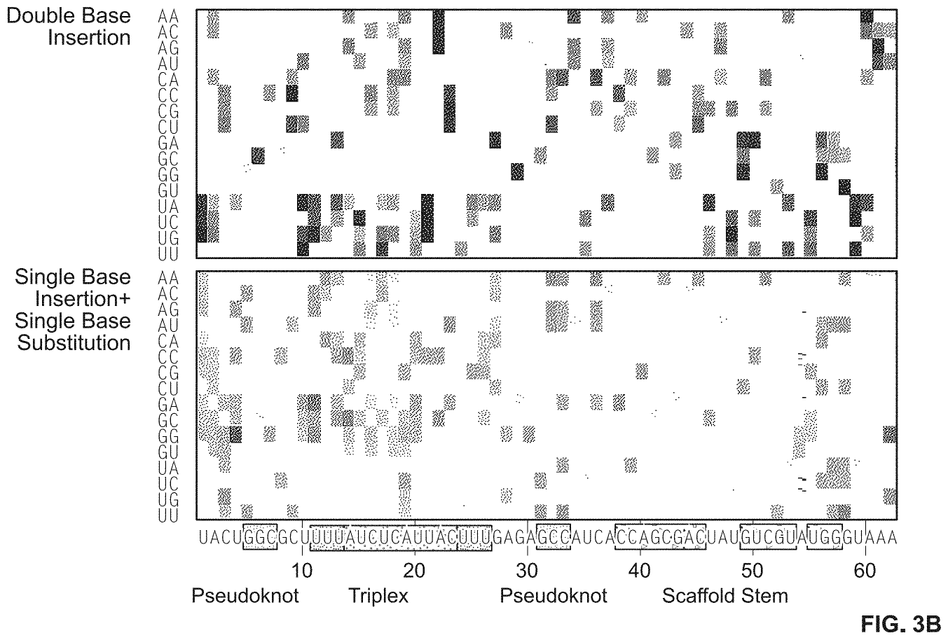

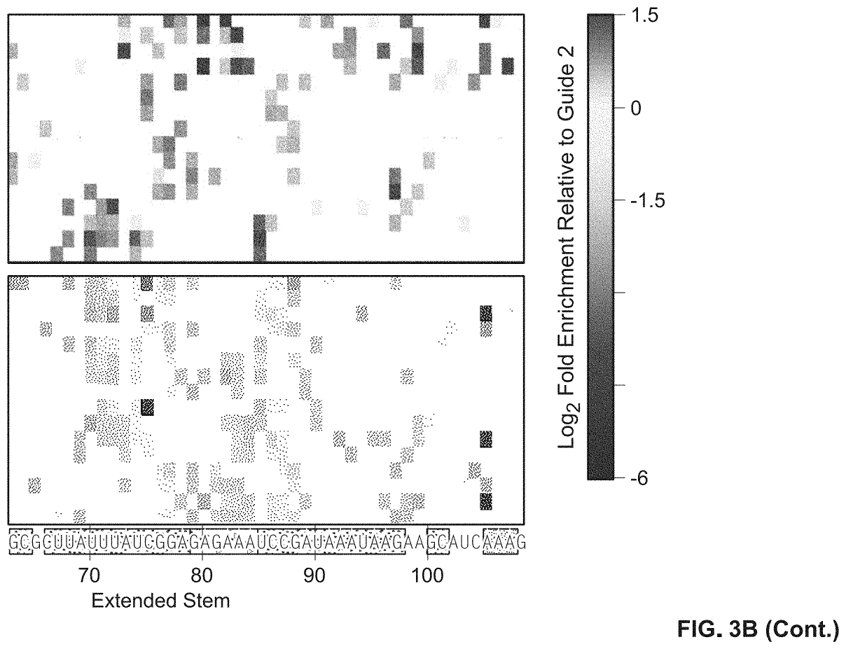

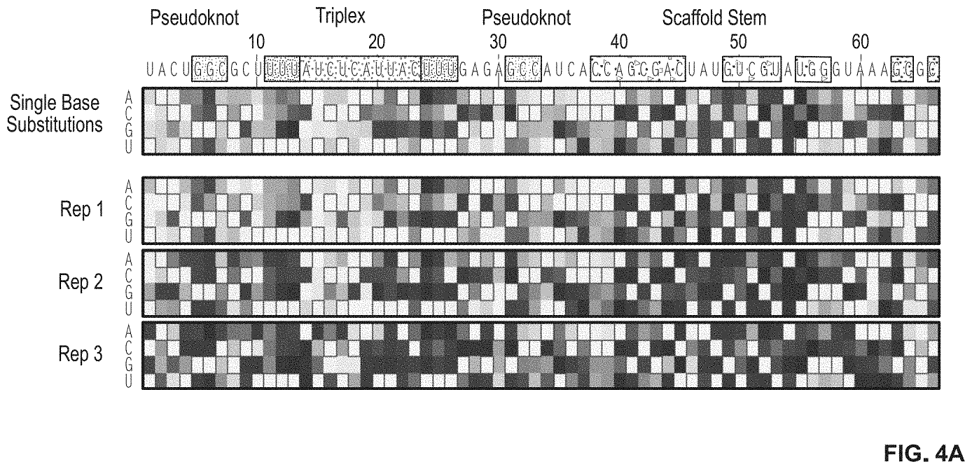

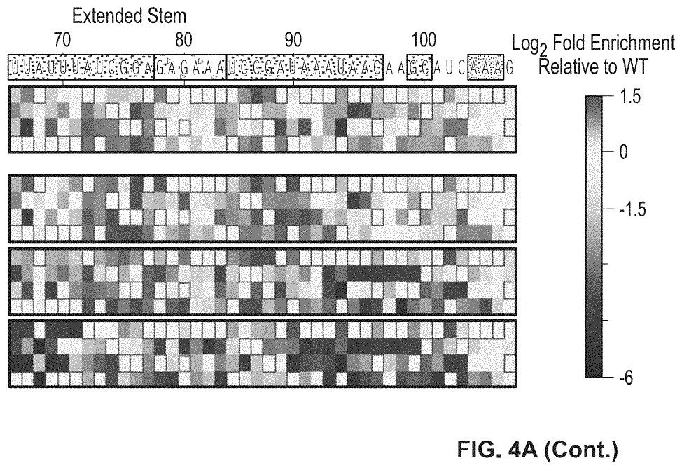

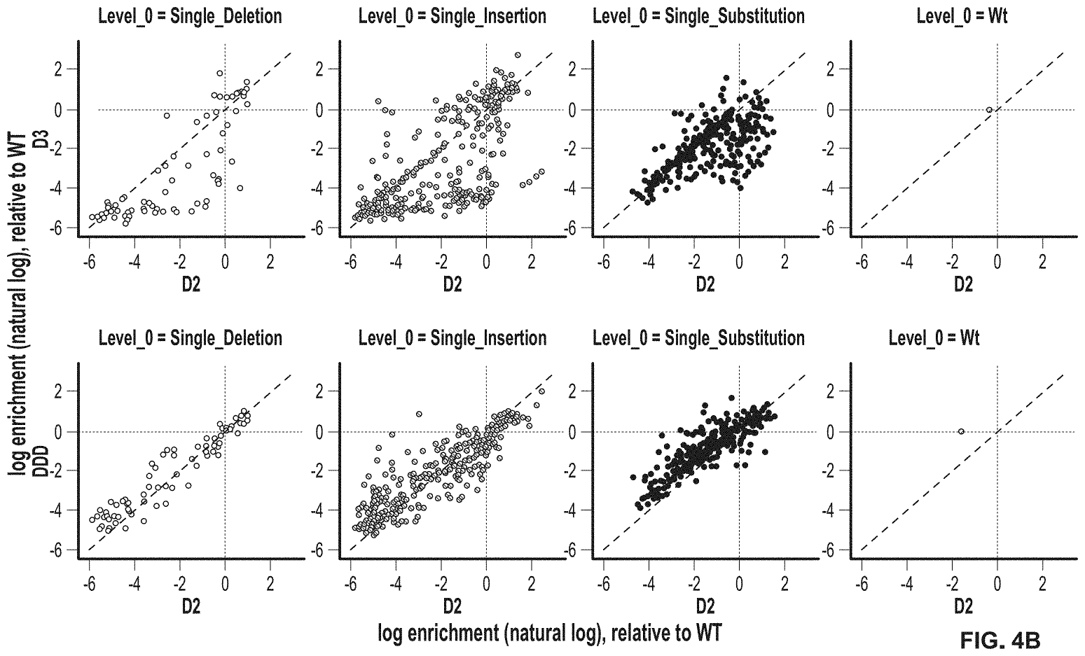

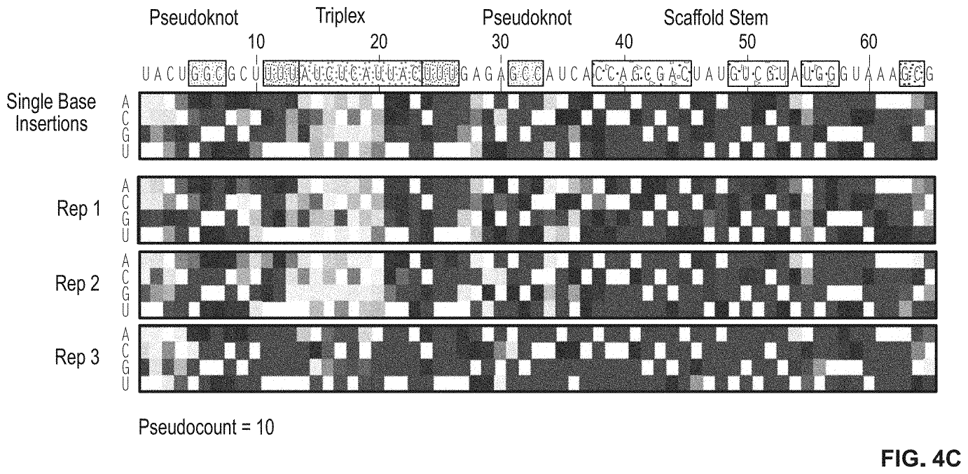

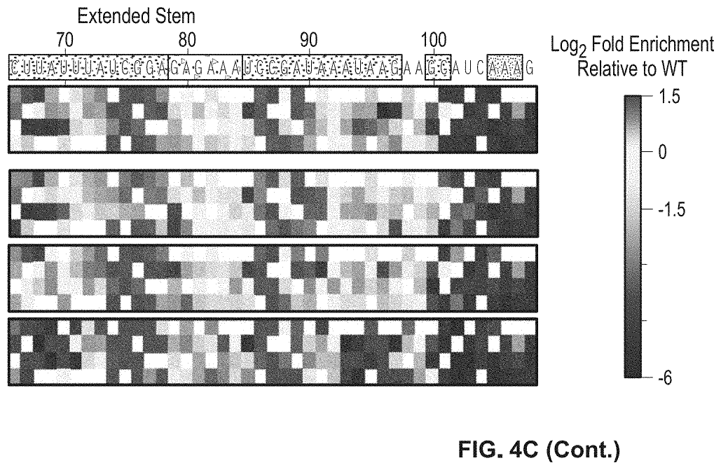

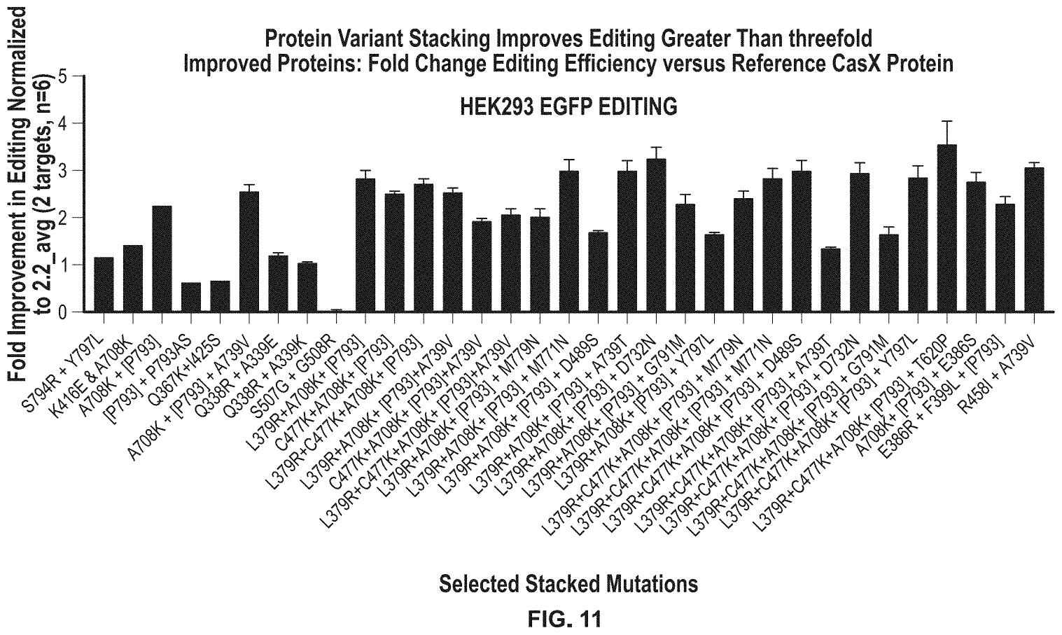

In another example, without wishing to be bound by any theory, constructs comprising four or more T nucleotides in row may be difficult to express in human expression systems. Screening a variant library comprising one or more variants in which a 4+ T region has been altered (e.g., by substitution) may demonstrate, in some embodiments, that certain substitutions do not have a detrimental effect on the desired characteristics of the biomolecule (such as solubility or activity). Such information can then be used, for example, to construct a variant in which a 4+ T region has been altered such that it is expected to be better suited to human expression systems, but without negatively affecting desirable positive characteristics. One exemplary such variant described herein includes the sgRNA with T10C alteration, used as the sgRNA in FIGS. 11A-C. The development of this sgRNA variant included information gleaned from the data shown in FIGS. 3A-3B, and 4A-4C, demonstrating that alteration of the T10 location did not have detrimental effects. Thus, this location could be substituted with a C, removing the 4T motif that is believed to have increased termination in human expression systems. Information obtained from the methods of variant and/or library construction and screening provided herein may, therefore, be combined with other information about the biomolecules and/or other alterations to construct new variants. Such additional alterations may include, for example, the addition of one or more functionalities (such as through protein fusions or combination with ribozymes) or removal of one or more regions of the protein (such as a stem truncation). Thus, the methods and compositions provided herein may, in some embodiments, provide information about regions of the biomolecule that are more highly mutable, which can be changed to a larger degree without loss of desirable characteristics, which could be subject to rational alterations (such as to install handles or additional functionality), or which can be removed, or any combinations thereof. The methods and compositions may also provide information about what alterations can be combined (e.g., “stacked”) in one or more additional variants, and/or additional libraries.

In some embodiments, the information obtained from the methods and compositions provided herein can be used, for example, to construct a variant nucleic acid (NA). In some embodiments, the variant NA is a guide NA. A guide NA (gNA) refers to a nucleic acid molecule that binds to a Cas protein or variant thereof, forming a nucleic acid-protein complex, and targets the complex to a specific location within a target nucleic acid (e.g., a target DNA). In some embodiments, the gNA is a deoxyribonucleic acid (DNA) molecule (a gDNA). In some embodiments, the gNA is a ribonucleic acid (RNA) molecule (a gRNA). In still further embodiments, the gNA comprises both deoxyribonucleotides and ribonucleotides. In some embodiments a guide NA is constructed based at least in part on information obtained using the methods and compositions described herein (e.g., screening an RNA library, or a DNA library, or both). In some embodiments, the guide NA is a single guide NA (sgNA). In some embodiments, the guide NA is a double guide NA (dgNA). In some embodiments, the guide NA binds to CasX, CasY, Cas9, Cas12a, Cas12b, Cas12c, Cas12f, Cas12g, Cas12h, Cas12i, Cas12j, Cas13a, Cas13b, Cas13c, Cas13d, Cas14, CASCADE, CSM, or CSY. In some embodiments, the guide NA binds to CasX, or CasY.

In certain embodiments of the methods provided herein, the method comprises one or more additional screening steps. For example, in some embodiments the at least a portion of the library identified in step (iii) is screened. In certain embodiments, the screen in (ii) and the screen of the at least a portion identified in step (iii) are different screen types (e.g., screen for different characteristics, or by different methods, or a combination thereof). In other embodiments, they are the same screen types. Evaluation of the libraries described herein is described in further detail below.

II. Library Evaluation

Once a library has been constructed, it is evaluated for one or more characteristics. Any suitable method of evaluation may be used, such that has sufficient throughput so as to map the number of individual mutations in the library (which may include, e.g., up to millions or billions of individual variants overall); and the method links phenotype and genotype. In some embodiments, methods with a low throughput may be used, for example, to evaluate a subpopulation of a library, or a small library targeting certain mutations, or a small library layering certain mutations of interest, or a focused library developed through multiple rounds of mutation and evaluation.

In some embodiments, the evaluation method uses living cells. Methods using living cells may, in some embodiments, be desirable because the effect of the genotype on the phenotype can be readily ascertained. Living cells may also be used to directly amplify sub-populations of the overall library.

An exemplary, but non-limiting DME screening assay comprises Fluorescence-Activated Cell Sorting (FACS). In some embodiments, FACS may be used to assay millions or up to billions of unique cells in a library. An exemplary FACS screening protocol comprises the following steps:

(1) PCR amplifying a purified plasmid library from the library construction phase. Flanking PCR primers can be designed that add appropriate restriction enzyme sites flanking the DNA encoding the biomolecule. Standard oligonucleotides can be used as PCR primers, and can be synthesized commercially. Commercially available PCR reagents can be used for the PCR amplification, and protocols should be performed according to the manufacturer's instructions. Methods of designing PCR primers, choice of appropriate restriction enzyme sites, selection of PCR reagents and PCR amplification protocols will be readily apparent to the person of ordinary skill in the art.

(2) The resulting PCR product is digested with the designed flanking restriction enzymes. Restriction enzymes may be commercially available, and methods of restriction enzyme digestion will be readily apparent to the person of ordinary skill in the art.

(3) The PCR product is ligated into a new DNA vector. Appropriate DNA vectors may include vectors that allow for the expression of the library in a cell. Exemplary vectors include, but are not limited to, lentiviral vectors, adenoviral vectors, adeno-associated viral (AAV) vectors and plasmids. This new DNA vector can be part of a protocol such as lentiviral integration in mammalian tissue culture, or a simple expression method such as plasmid transformation in bacteria. Any vectors that allow for the expression of the biomolecule, and the library of variants thereof, in any suitable cell type, are considered within the scope of the disclosure. Cell types may include bacterial cells, yeast cells, and mammalian cells. Exemplary bacterial cell types may include E. coli. Exemplary yeast cell types may include Saccharomyces cerevisiae. Exemplary mammalian cell types may include mouse, hamster, and human cell lines, such as HEK293 cells. Choice of vector and cell type will be readily apparent to the person of ordinary skill in the art. DNA ligase enzymes can be purchased commercially, and protocols for their use will also be readily apparent to one of ordinary skill in the art.

(4) Once the library has been cloned into a vector suitable for in vivo expression, the library is screened. If the biomolecule has a function which alters fluorescent protein production in a living cell, the biomolecule's biochemical function will be correlated with the fluorescence intensity of the cell overall. By observing a population of millions of cells on a flow cytometer, a library can be seen to produce a broad distribution of fluorescence intensities. Individual sub-populations from this overall broad distribution can be extracted by FACS. For example, if the function of the biomolecule is to repress expression of a fluorescent protein, the least bright cells will be those expressing biomolecules whose function has been improved by DME. Alternatively, if the function of the biomolecule is to increase expression of a fluorescent protein, the brightest cells will be those expressing biomolecules whose function has been improved by DME. Cells can be isolated based on fluorescence intensity by FACS and grown separately from the overall population.

(5) After FACS sorting cells expressing a library of biomolecule variants, cultures comprising the original library and/or only highly functional biomolecule variants, as determined by FACS sorting, can be amplified separately. If the cells that were FACS sorted comprise cells that express the library of biomolecule variants from a plasmid (for example, E. coli cells transformed with a plasmid expression vector), these plasmids can be isolated, for example through miniprep. Conversely if the library of biomolecule variants has been integrated into the genomes of the FACs sorted cells, this DNA region can be PCR amplified and, optionally, subcloned into a suitable vector for further characterization using methods known in the art. Thus, the end product of library screening is a DNA library representing the initial, or ‘naive’, library, as well as one or more DNA libraries containing sub-populations of the naive library which comprise highly functional mutant variants of the biomolecule identified by the screening processes described herein.

In some embodiments, a biomolecule library that has been screened or selected for one or more variants are further characterized. For example, in some embodiments, a library has one or more highly functional variants which are further characterized to gain insight into possible mutational correlations or relationships that lead to a desired functional change. In some embodiments, further characterizing the library comprises analyzing variants individually through sequencing, such as Sanger sequencing, to identify the specific mutation or mutations that are connected to the change in characteristic (such as a highly functional characteristic). Individual mutant variants of the biomolecule can be isolated through standard molecular biology techniques for later analysis of function.

In some embodiments, further characterizing the library comprises high throughput sequencing of both the entire, original library (the “naïve” library, e.g. the library in step (i)) and the one or more sub-populations of highly functional variants (e.g., a library of step (iii)). This approach may, in some embodiments, allow for the rapid identification of mutations that are over-represented in the one or more sub-populations of highly functional variants compared to a naïve library. Without wishing to be bound by any theory, mutations that are over-represented in the one or more sub-populations of highly functional variants may be responsible for the activity of the highly functional variants. In some embodiments, further characterizing the library comprises both sequencing of individual variants and high throughput sequencing of both the naïve library and the one or more sub-populations of highly functional variants.

High throughput sequencing can produce high throughput data indicating the functional effect of the library members. In embodiments wherein one or more libraries represents every possible mutation of every monomer location, such high throughput sequencing can evaluate the functional effect of every possible mutation. Such sequencing can also be used to evaluate one or more highly functional sub-populations of a given library, which in some embodiments may lead to identification of mutations that result in improved function. An exemplary protocol for high throughput sequencing of a library with a highly functional sub-population is as follows:

(1) High throughput sequence the naïve library (N). High throughput sequence the highly functional sub-population library (F). Any high throughput sequencing platform that can generate a suitable abundance of reads can be used. Exemplary sequencing platforms include, but are not limited to Illumina, Ion Torrent, 454 and PacBio sequencing platforms.

(2) Select a particular mutation to evaluate (i). Calculate the total fractional abundance of i in N (i(N)). Calculate the total fractional abundance of i in F, (i(F)).

(3) Calculate the following: [(i(F)+1)/(i(N)+1)]. This value, the ‘enrichment ratio’, is correlated with the function of the particular mutant variant i of the biomolecule. Other methods of calculating enrichment may also be used (e.g., pseudocount).

(4) Calculate the enrichment ratio for each of the mutations observed in deep sequencing of the library.

(5) The set of enrichment ratios for the entire library can be converted to a log scale and rescaled such that all values range between −1 and 1, where a value of 0 represents no enrichment (i.e. an enrichment ratio of 1). These rescaled values can be referred to as the relative ‘fitness’ of any particular mutation. These fitness values quantitatively indicate the effect a particular mutation has on the biochemical function of the biomolecule.

(6) The set of calculated fitness values can be mapped to visually represent the fitness landscape of all possible mutations to a biomolecule. The fitness values can also be rank ordered to determine the most beneficial mutations contained within the library. Other analysis methods could also be used separately or in combination. For example, machine learning could be used to predict the effects of untested mutations or to determine specification locations and/or mutations that have the greatest effect.

III. Iterating DME

In some embodiments, a highly functional variant produced by DME has more than one mutation. For example, combinations of different mutations can in some embodiments produce optimized biomolecules whose function is further improved by the combination of mutations. In some embodiments, the effect of combining mutations on the function of a biomolecule is additive. As used herein, a combination of mutations that is additive refers to a combination whose effect on function is equal to the sum of the effects of each individual mutation when assayed in isolation. In some embodiments, the effect of combining mutations on function of the biomolecule is synergistic. As used herein, a combination of mutations that is synergistic refers to a combination whose effect on function is greater than the sum of the effects of each individual mutation when assayed in isolation. Other mutations may exhibit additional unexpected nonlinear additive effects, or even negative effects; this phenomenon is referred to herein as epistasis.

Epistasis can be unpredictable, and can be a significant source of variation when combining mutations. Epistatic effects can, in some embodiments, be addressed through additional high throughput experimental methods in library construction and evaluation. In some embodiments, the entire library construction and evaluation protocol can be iterated, returning to the library construction step and selecting only mutations identified as having desired effects (such as increased functionality) from an initial library screen. Thus, in some embodiments, library construction and screening is iterated, with one or more cycles focusing the library on a sub-population or sub-populations of mutations having one or more desired effects. In such embodiments, layering of selected mutations may lead to improved variants. In certain embodiments, mutations that lead to different improved effects are layered, such that a variant may have two or more improved characteristics compared to the reference biomolecule. In some alternative embodiments, the process can be repeated with the full set of mutations, but targeting a novel, pre-mutated version of the biomolecule. For example, one or more highly functional variants identified in a first round of library construction, evaluation, and characterization can be used as the target for further rounds using a broad, unfocused set of further mutations (such as every possible mutation, or a subset thereof), and the process repeated. Any number, type of iterations or combinations of iterations are envisaged as within the scope of the disclosure.

Thus, in some aspects, provided herein is an iterative method of selecting an improved biomolecule variant, wherein the biomolecule is a protein, DNA, or RNA, comprising:
(i) constructing a library comprising a plurality of biomolecule variants, wherein each variant is independently a variant of the same reference biomolecule;
(ii) screening the library of (i);
(iii) identifying at least a portion of the library of (i) that exhibits one or more improved characteristics compared to the reference biomolecule;
(iv) carrying out one or more additional rounds of library construction and screening, wherein construction of each library comprises:
   altering one or more additional monomer locations of the identified portion of the previous library to produce a subsequent library of biomolecule variants; and
(iv) selecting the improved biomolecule variant from the final library of biomolecule variants, wherein the improved biomolecule variant exhibits one or more improved characteristics compared to the reference biomolecule.

The library of (i) may be any variant library described herein, such as:
wherein each variant comprises an alteration of one or more monomer locations of the reference biomolecule, wherein the monomer is an amino acid of the protein or nucleotide of the RNA or DNA, and
wherein each alteration of a monomer location is independently selected from the group consisting of substitution of the monomer, deletion of one or more consecutive monomers beginning at the location, and insertion of one or more consecutive monomers adjacent to the location;
wherein the library represents variants comprising alteration of one or more locations for at least 10% of the monomer locations of the reference biomolecule

In some embodiments, an iterative method comprises one additional round, two additional rounds, three additional rounds, four additional rounds, five additional rounds, or more of library construction and screening. In certain embodiments, each subsequent library is smaller than the previous library, for example wherein evolution of the variants is directed to a particular mutation or theme of mutations. In other embodiments, each library is of approximately the same size, for example within about 1%, within about 5%, within about 10%, or within about 15% of the previous or subsequent, or both, libraries. In still further embodiments, each library is of an independent size.

In certain embodiments, one or more alterations of the biomolecule variants in the variant library being screened, or, if more than one library is screened (e.g., in multiple rounds, and/or iterative processes), one or more alterations of biomolecule variants in one or more libraries, is independently an alteration deriving from rational design. In some embodiments, one or more alterations is random. In certain embodiments, a combination of rational alterations (e.g., altering, including removing, one or more motifs present in the reference sequence based on a specific structural or functional analysis or theory).

In some embodiments, the DME methods provided herein comprise further modification to one or more variants of a library using rational mutagenesis, and then optionally evaluating said modifications. For example, in some embodiments, without wishing to be bound by any theory, four T ribonucleotides in a row may cause termination in a human cell expression system. Thus, for example, in some embodiments one or more variants is selected through the methods provided herein, and then the one or more variants is evaluated for the presence of four T ribonucleotides in the sequence, and identified variants are modified to remove such repeats. In some embodiments, these further modified variants are evaluated.

IV. Reference Biomolecule

Any suitable reference protein, RNA, or DNA may be used as the reference biomolecule in the methods and compositions described herein. In some embodiments, the reference biomolecule is a naturally occurring protein, RNA, or DNA. In other embodiments, the reference biomolecule is not naturally occurring.

In some embodiments, the reference biomolecule is a protein. In certain embodiments, the reference biomolecule is a CRISPR/Cas family endonuclease (Cas protein), for example one that interacts with a guide RNA (gRNA) to form a ribonucleoprotein (RNP) complex. In some embodiments, the RNP is capable of cleaving DNA. In some embodiments, the RNP is capable of cleaving RNA. In certain embodiments, the RNP complex can be targeted to a particular site in a target nucleic acid via base pairing between the gRNA and a target sequence in the target nucleic acid.

In some embodiments, the CRISPR/Cas protein is a Class 1 protein, e.g. a Type I, Type III, or Type IV protein. In some embodiments, the CRISPR/Cas protein is a Class II protein, e.g., a Type II, Type V, or Type VI protein.

Any suitable Cas protein may be used. For example, in some embodiments, the Cas protein is CasX, CasY, Cas9, Cas12a, Cas12b, Cas12c, Cas12f, Cas12g, Cas12h, Cas12i, Cas12j, Cas13a, Cas13b, Cas13c, Cas13d, Cas14, CASCADE, CSM, or CSY. In some embodiments, the Cas protein is CasX. In certain embodiments, the Cas protein is CasY.

In some embodiments, the reference CasX protein is a naturally-occurring protein. For example, reference CasX proteins can, in some embodiments, be isolated from naturally occurring prokaryotic cells, such as cells of Deltaproteobacter, Planctomycetes, or Candidatus Sungbacteria species. In other embodiments, the reference CasX protein is not a naturally-occurring protein.

In some embodiments, the reference biomolecule is a CasX protein isolated or derived from Deltaproteobacter. In some embodiments, the reference biomolecule is a CasX protein isolated or derived from Planctomycetes. In some embodiments, the reference biomolecule is a CasX protein isolated or derived from Candidatus Sungbacteria. In some embodiments, the reference biomolecule comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical or 100% identical to a sequence of SEQ ID NO: 1, SEQ ID NO: 2, or SEQ ID NO: 3.

| (SEQ ID NO: 1) | |
|---|---|
| 1 | MEKRINKIRK KLSADNATKP VSRSGPMKTL LVRVMTDDLK KRLEKRRKKP EVMPQVISNN |
| 61 | AANNLRMLLD DYTKMKEAIL QVYWQEFKDD HVGLMCKFAQ PASKKIDQNK LKPEMDEKGN |
| 121 | LTTAGFACSQ CGQPLFVYKL EQVSEKGKAY TNYFGRCNVA EHEKLILLAQ LKPEKDSDEA |
| 181 | VTYSLGKFGQ RALDFYSIHV TKESTHPVKP LAQIAGNRYA SGPVGKALSD ACMGTIASFL |
| 241 | SKYQDIIIEH QKVVKGNQKR LESLRELAGK ENLEYPSVTL PPQPHTKEGV DAYNEVIARV |
| 301 | RMWVNLNLWQ KLKLSRDDAK PLLRLKGFPS FPVVERRENE VDWWNTINEV KKLIDAKRDM |
| 361 | GRVFWSGVTA EKRNTILEGY NYLPNENDHK KREGSLENPK KPAKRQFGDL LLYLEKKYAG |
| 421 | DWGKVFDEAW ERIDKKIAGL TSHIEREEAR NAEDAQSKAV LTDWLRAKAS FVLERLKEMD |
| 481 | EKEFYACEIQ LQKWYGDLRG NPFAVEAENR VVDISGFSIG SDGHSIQYRN LLAWKYLENG |
| 541 | KREFYLLMNY GKKGRIRFTD GTDIKKSGKW QGLLYGGGKA KVIDLTFDPD DEQLIILPLA |
| 601 | FGTRQGREFI WNDLLSLETG LIKLANGRVI EKTIYNKKIG RDEPALFVAL TFERREVVDP |
| 661 | SNIKPVNLIG VDRGENIPAV IALTDPEGCP LPEFKDSSGG PTDILRIGEG YKEKQRAIQA |
| 721 | AKEVEQRRAG GYSRKFASKS RNLADDMVRN SARDLFYHAV THDAVLVFEN LSRGFGRQGK |
| 781 | RTFMTERQYT KMEDWLTAKL AYEGLTSKTY LSKTLAQYTS KTCSNCGFTI TTADYDGMLV |
| 841 | RLKKTSDGWA TTLNNKELKA EGQITYYNRY KRQTVEKELS AELDRLSEES GNNDISKWTK |
| 901 | GRRDEALFLL KKRFSHRPVQ EQFVCLDCGH EVHADEQAAL NIARSWLFLN SNSTEFKSYK |
| 961 | SGKQPFVGAW QAFYKRRLKE VWKPNA. |
| (SEQ ID NO: 2) | |
| 1 | MQEIKRINKI RRRLVKDSNT KKAGKTGPMK TLLVRVMTPD LRERLENLRK KPENIPQPIS |
| 61 | NTSRANLNKL LTDYTEMKKA ILHVYWEEFQ KDPVGLMSRV AQPAPKNIDQ RKLIPVKDGN |
| 121 | ERLTSSGFAC SQCCQPLYVY KLEQVNDKGK PHTNYFGRCN VSEHERLILL SPHKPEANDE |
| 181 | LVTYSLGKFG QRALDFYSIH VTRESNHPVK PLEQIGGNSC ASGPVGKALS DACMGAVASF |
| 241 | LTKYQDIILE HQKVIKKNEK RLANLKDIAS ANGLAFPKIT LPPQPHTKEG IEAYNNVVAQ |
| 301 | IVIWVNLNLW QKLKIGRDEA KPLQRLKGFP SFPLVERQAN EVDWWDMVCN VKKLINEKKE |
| 361 | DGKVFWQNLA GYKRQEALLP YLSSEEDRKK GKKFARYQFG DLLLHLEKKH GEDWGKVYDE |
| 421 | AWERIDKKVE GLSKEIKLEE ERRSEDAQSK AALTDWLRAK ASFVIEGLKE ADKDEFCRCE |
| 481 | LKLQKWYGDL RGKPFAIEAE NSILDISGFS KQYNCAFIWQ KDGVKKLNLY LIINYFKGGK |
| 541 | LRFKKIKPEA FEANRFYTVI NKKSGEIVPM EVNFNFDDPN LIILPLAFGK RQGREFIWND |
| 601 | LLSLETGSLK LANGRVIEKT LYNRRTRQDE PALFVALTFE RREVLDSSNI KPMNLIGIDR |
| 661 | GENIPAVIAL TDPEGCPLSR FKDSLGNPTH ILRIGESYKE KQRTIQAAKE VEQRRAGGYS |
| 721 | RKYASKAKNL ADDMVRNTAR DLLYYAVTQD AMLIFENLSR GFGRQGKRTF MAERQYTRME |
| 781 | DWLTAKLAYE GLPSKTYLSK TLAQYTSKTC SNCGFTITSA DYDRVLEKLK KTATGWMTTI |
| 841 | NGKELKVEGQ ITYYNRYKRQ NVVKDLSVEL DRLSEESVNN DISSWTKGRS GEALSLLKKR |
| 901 | FSHRPVQEKF VCLNCGFETH ADEQAALNIA RSWLFLRSQE YKKYQTNKTT GNTDKRAFVE |
| 961 | TWQSFYRKKL KEVWKPAV. |
| (SEQ ID NO: 3) | |
| 1 | MDNANKPSTK SLVNTTRISD HFGVTPGQVT RVESEGIIPT KRQYAIIERW FAAVEAARER |
| 61 | LYGMLYAHFQ ENPPAYLKEK FSYETFFKGR PVLNGLRDID PTIMTSAVFT ALRHKAEGAM |
| 121 | AAFHTNHRRL FEEARKKMRE YAECLKANEA LLRGAADIDW DKIVNALRTR LNTCLAPEYD |
| 181 | AVIADFGALC AFRALIAETN ALKGAYNHAL NQMLPALVKV DEPEEAEESP RLRFFNGRIN |
| 241 | DLPKFPVAER ETPPDTETII RQLEDMARVI PDTAEILGYI HRIRHKAARR KPGSAVPLPQ |
| 301 | RVALYCAIRM ERNPEEDPST VAGHFLGEID RVCEKRRQGL VRTPFDSQIR ARYMDIISFR |
| 361 | ATLAHPDRWT EIQFLRSNAA SRRVRAETIS APFEGFSWTS NRTNPAPQYG MALAKDANAP |
| 421 | ADAPELCICL SPSSAAFSVR EKGGDLIYMR PTGGRRGKDN PGKEITWVPG SFDEYPASGV |
| 481 | ALKLRLYFGR SQARRMLTNK TWGLLSDNPR VFAANAELVG KKRNPQDRWK LFFHMVISGP |
| 541 | PPVEYLDFSS DVRSRARTVI GINRGEVNPL AYAVVSVEDG QVLEEGLLGK KEYIDQLIET |
| 601 | RRRISEYQSR EQTPPRDLRQ RVRHLQDTVL GSARAKIHSL IAFWKGILAI ERLDDQFHGR |
| 661 | EQKIIPKKTY LANKTGFMNA LSFSGAVRVD KKGNPWGGMI EIYPGGISRT CTQCGTVWLA |
| 721 | RRPKNPGHRD AMVVIPDIVD DAAATGFDNV DCDAGTVDYG ELFTLSREWV RLTPRYSRVM |
| 781 | RGTLGDLERA IRQGDDRKSR QMLELALEPQ PQWGQFFCHR CGFNGQSDVL AATNLARRAI |
| 841 | SLIRRLPDTD TPPTP. |

A polynucleotide or polypeptide can have a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST.

In other embodiments, the reference biomolecule is RNA. In some embodiments, the reference biomolecule is a CRISPR guide RNA. CRISPR guide RNAs (gRNA) include ribonucleic acid molecules that bind to a Cas protein, forming a ribonucleoprotein complex (RNP), and targets the complex to a specific location within a target nucleic acid (e.g., a target DNA or target RNA). In some embodiments, the gRNA is naturally occurring. In other embodiments, the gRNA is not naturally occurring.

The “spacer”, also sometimes referred to as “targeting” sequence of a gRNA, can in some embodiments be modified so that the gRNA can target a Cas protein to any desired sequence of any desired target nucleic acid, with the exception (e.g., as described herein) that the PAM sequence can be taken into account. Thus, for example, a gRNA may in some embodiments have a spacer sequence with complementarity to (e.g., can hybridize to) a sequence in a nucleic acid in a eukaryotic cell, e.g., a eukaryotic nucleic acid (e.g., a eukaryotic chromosome, chromosomal sequence, a eukaryotic RNA, etc.) that is adjacent to a sequence complementary to a PAM sequence. In some embodiments, the spacer of a gRNA has between 14 and 35 consecutive nucleotides. In some embodiments, the spacer has 14, 15, 16, 18, 18, 19, 20, 21, 22, 23 24, 25, 26, 27, 28, 29, 30, 31, 32, 33, 34 or 35 consecutive nucleotides. In some embodiments, the spacer sequence can comprise 0 to 5, 0 to 4, 0 to 3, or 0 to 2 mismatches relative to the target nucleic acid sequence and retain sufficient binding specificity such that the RNP comprising the gRNA comprising the spacer sequence can form a complementary bond with respect to the target nucleic acid.

In some embodiments, a gRNA can include two segments, a targeting segment and a protein-binding segment (constituting the scaffold discussed below); in some embodiments, the segments are fused. The targeting segment of a gRNA includes a nucleotide sequence (a guide sequence) that is complementary to (and therefore hybridizes with) a specific sequence (a target site) within a target nucleic acid (e.g., a target ssRNA, a target ssDNA, the complementary strand of a double stranded target DNA, etc.). The protein-binding segment (or “protein-binding sequence”) interacts with (e.g., binds to) a Cas protein. In those embodiments where the gRNA includes two segments, the protein-binding segment of the gRNA includes two complementary stretches of nucleotides that hybridize to one another to form a double stranded RNA duplex (dsRNA duplex). Site-specific binding and/or cleavage of a target nucleic acid (e.g., genomic DNA) can occur at one or more locations (e.g., target sequence of a target nucleic acid) determined by base-pairing complementarity between the gRNA (the guide sequence of the g RNA) and the target nucleic acid. A gRNA and a Cas protein may form a complex (e.g., bind via non-covalent interactions), and the gRNA may provide target specificity to the complex by including a guide sequence (a nucleotide sequence that is complementary to a sequence of a target nucleic acid). The guide sequence is sometimes referred to herein as the “spacer” or “spacer sequence.” The Cas protein of the complex may provide the site-specific activity (e.g., cleavage activity provided by the Cas protein). In other words, in some embodiments the Cas protein is guided to a target nucleic acid sequence (e.g. a target sequence) by virtue of its association with the Cas gRNA.

In some embodiments, a gRNA includes an “activator” and a “targeter” (e.g., an “activator-RNA” and a “targeter-RNA,” respectively). When the “activator” and a “targeter” are two separate molecules, the reference gRNA may be referred to, for example, as a “dual guide RNA”, a “dgRNA,” a “double-molecule guide RNA”, or a “two-molecule guide RNA”. The term “targeter” or “targeter RNA” is used herein to refer to a crRNA-like molecule (crRNA: “CRISPR RNA”) of a Cas guide RNA (e.g., a dgRNA; or, when the “activator” and the “targeter” are linked together, a single guide RNA (sgRNA)). Thus, for example, a reference gRNA (dgRNA or sgRNA) comprises a guide sequence and a duplex-forming segment (e.g., a duplex forming segment of a crRNA, which can also be referred to as a crRNA repeat). Because the sequence of a guide sequence (the segment that hybridizes with a target sequence of a target nucleic acid) of a targeter may be modified by a user to hybridize with a desired target nucleic acid, the sequence of a targeter may be a non-naturally occurring sequence. A targeter comprises both the guide sequence (aka spacer sequence) of the gRNA and a stretch of nucleotides that forms one half of the dsRNA duplex of the protein-binding segment of the gRNA. A corresponding trans-activating crRNA (tracrRNA)-like molecule (activator) comprises a stretch of nucleotides (a duplex-forming segment) that forms the other half of the dsRNA duplex of the protein-binding segment of the gRNA. In some embodiments, a targeter and an activator (as a corresponding pair) hybridize to form a dsRNA. In some embodiments, the activator and targeter of a gRNA are covalently linked to one another (e.g., via intervening nucleotides) and the gRNA is referred to herein as a “single guide RNA”, an “sgRNA,” a “single-molecule guide RNA,” or a “one-molecule guide RNA”. Thus, a sgRNA, in some embodiments, comprises a targeter (e.g., targeter-RNA) and an activator (e.g., activator-RNA) that are linked to one another (e.g., covalently by intervening nucleotides), and hybridize to one another to form the double stranded RNA duplex (dsRNA duplex) of the protein-binding segment of the guide RNA, resulting in a stem-loop structure. In some embodiments, the targeter and the activator each have a duplex-forming segment, where the duplex forming segment of the targeter and the duplex-forming segment of the activator have complementarity with one another and hybridize to one another.

In some embodiments, the linker covalently attaching the targeter and the activator is a stretch of nucleotides. Exemplary linkers may include, but are not limited to GAAA, GAGAAA, and CUUCGG. In some embodiments, the linker is CUUCGG. In some cases, the targeter and activator of a sgRNA are linked to one another by intervening nucleotides, and the linker has a length of from 3 to 20 nucleotides (nt) (e.g., from 3 to 15, 3 to 12, 3 to 10, 3 to 8, 3 to 6, 3 to 5, 3 to 4, 4 to 20, 4 to 15, 4 to 12, 4 to 10, 4 to 8, 4 to 6, or 4 to 5 nt). In some embodiments, the linker of a sgRNA has a length of from 3 to 100 nucleotides (nt) (e.g., from 3 to 80, 3 to 50, 3 to 30, 3 to 25, 3 to 20, 3 to 15, 3 to 12, 3 to 10, 3 to 8, 3 to 6, 3 to 5, 3 to 4, 4 to 100, 4 to 80, 4 to 50, 4 to 30, 4 to 25, 4 to 20, 4 to 15, 4 to 12, 4 to 10, 4 to 8, 4 to 6, or 4 to 5 nt). In some embodiments, the linker of a sgRNA has a length of from 3 to 10 nucleotides (nt) (e.g., from 3 to 9, 3 to 8, 3 to 7, 3 to 6, 3 to 5, 3 to 4, 4 to 10, 4 to 9, 4 to 8, 4 to 7, 4 to 6, or 4 to 5 nt).

In some embodiments, the reference CRISPR guide RNA is a single guide RNA (sgRNA), for example a sgRNA that binds to CasX, CasY, Cas9, Cas12a, Cas12b, Cas12c, Cas12f, Cas12g, Cas12h, Cas12i, Cas12j, Cas13a, Cas13b, Cas13c, Cas13d, Cas14, CASCADE, CSM, or CSY. In certain embodiments, the CRISPR guide RNA is a single guide RNA that binds CasX. In some embodiments, the CasX is of SEQ ID NO: 1, SEQ ID NO: 2, or SEQ ID NO: 3. In other embodiments, the CRISPR guide RNA is an sgRNA that binds CasY.

In some embodiments, the reference gRNA comprises a sequence of a naturally-occurring gRNA. In some embodiments, the reference biomolecule is a guide RNA comprising sequence isolated or derived from Deltaproteobacter. In some embodiments, the sequence is a tracrRNA sequence, for example a CasX tracrRNA sequence. Exemplary CasX reference tracrRNA sequences isolated or derived from Deltaproteobacter may include:

(SEQ ID NO: 239)
UUAUUCCAUUACUUUGGAGCCAGUCCCAGCGACUAUGUCGUAUGGACGA
AGCGCUUAUUUAUCGGAGA
and
(SEQ ID NO: 240)
UUAUUCCAUUACUUUGGAGCCAGUCCCAGCGACUAUGUCGUAUGGACGA
AGCGCUUAUUUAUCGG.

Exemplary crRNA sequences isolated or derived from Deltaproteobacter may comprise a sequence of:

(SEQ ID NO: 241)
CCGAUAAGUAAAACGCAUCAAAG.

In some embodiments, the reference biomolecule is a gRNA comprising a sequence isolated or derived from Planctomycetes. In some embodiments, the sequence is a tracrRNA sequence, such as a CasX tracrRNA sequence. Exemplary CasX reference tracrRNA sequences isolated or derived from Planctomycetes may include:

(SEQ ID NO: 242)
UUAUCUCAUUACUUUGAGAGCCAUCACCAGCGACUAUGUCGUAUGGGUA
AAGCGCUUAUUUAUCGGAGA
and
(SEQ ID NO: 243)
UUAUCUCAUUACUUUGAGAGCCAUCACCAGCGACUAUGUCGUAUGGGUA
AAGCGCUUAUUUAUCGG.

Exemplary crRNA sequences isolated or derived from Planctomycetes may comprise a sequence of:

(SEQ ID NO: 244)
UCUCCGAUAAAUAAGAAGCAUCAAAG

In some embodiments, the reference biomolecule is a gRNA comprising a sequence isolated or derived from Candidatus Sungbacteria. In some embodiments, the sequence is a tracrRNA sequence, such as a CasX tracrRNA sequence. Exemplary CasX tracrRNA sequences isolated or derived from Candidatus Sungbacteria may include:

(SEQ ID NO: 245)
UAAAUUUUUUGAGCCCUAUCUCCGCGAGGAAGACAGGGCUCUUUUCAUG
AGAGGAAGCUUUUAUACCCGACCGGUAAUCCGGUCGGGGGAUUGGCCGU
UGAAACGAUUUUAAAGCGGCCAAUGGGCCCCUCUAUAUGGAUACUACUU
AUAUAAGGAGCUUGGGGAAGAAGAUAGCUUAAUCCCGCUAUCUUGUCAA
GGGGUUGGGGGAGUAUCAGUAUCCGGCAGGCGCC.

Exemplary crRNA sequences isolated or derived from Candidatus Sungbacteria may comprise sequences of

(SEQ ID NO: 10)
GUUUACACACUCCCUCUCAUAGGGU,
(SEQ ID NO: 11)
GUUUACACACUCCCUCUCAUGAGGU,
(SEQ ID NO: 12)
UUUUACAUACCCCCUCUCAUGGGAU
and
(SEQ ID NO: 13)
GUUUACACACUCCCUCUCAUGGGGG,
and
(SEQ ID NO: 246)
GUUUACACACUCCCUCUCAUAGGG

In some embodiments, the reference biomolecule is a gRNA comprising a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical or 100% identical to a sequence isolated or derived from Deltaproteobacter, Candidatus Sungbacteria, or Planctomycetes.

In some embodiments, the reference biomolecule is a reference gRNA that is a capable of forming a complex with Cas12a.

In some embodiments, the reference biomolecule is a reference gRNA comprising a sequence that is not naturally occurring, for example a chimeric or fusion sequence.

In some embodiments, the reference biomolecule is a CasX sgRNA comprising a sequence of:

(SEQ ID NO: 4)
ACAUCUGGCGCGUUUAUUCCAUUACUUUGGAGCCAGUCCCAGCGACUAU
GUCGUAUGGACGAAGCGCUUAUUUAUCGGAGAgaaaCCGAUAAGUAAAA
CGCAUCAAAG.

In some embodiments, the reference biomolecule is a CasX sgRNA comprising the sequence of:

(SEQ ID NO: 5)
UACUGGCGCUUUUAUCUCAUUACUUUGAGAGCCAUCACCAGCGACUAUG
UCGUAUGGGUAAAGCGCUUAUUUAUCGGAGAGAAAUCCGAUAAAUAAGA
AGCAUCAAAG.

In some embodiments, the reference biomolecule is a CasX sgRNA comprising a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical or 100% identical to SEQ ID NO: 4, or SEQ ID NO: 5.

V. Variants

In still further aspects, also provided herein are variants selected by the methods described herein. In some embodiments, the variant has one or more improved characteristics compared to the reference biomolecule.

In some embodiments, the variant is a protein, and the one or more improved characteristics are independently selected from the group consisting of improved folding, improved stability, improved activity, improved protein solubility, improved binding to a binding partner, improved stability of a protein:binding partner complex, and improved yield.

In certain embodiments, the variant is a CRISPR associated protein, (e.g., a CasX variant protein) and the one or more improved characteristics are independently selected from the group consisting of improved folding of the variant, improved binding affinity to the guide RNA, improved binding affinity to a target DNA, altered binding affinity to or ability to utilize one or more PAM sequences for the editing of a target DNA, improved unwinding of a target DNA, increased activity, improved editing efficiency, improved editing specificity, increased activity of the nuclease, increased target strand loading for double strand cleavage, decreased target strand loading for single strand nicking, decreased off-target cleavage, decreased off-target binding/nicking, improved binding of the non-target strand of a DNA, improved protein stability, improved protein:guide NA complex stability, improved protein solubility, improved protein:guide RNA complex stability, improved protein yield, increased collateral activity, and decreased collateral activity. In some embodiments, a target DNA is dsDNA. In other embodiments, a target DNA is ssDNA.

In a particular feature, the methods of the disclosure result in CasX variant protein with the ability to utilize a larger spectrum of PAM sequences for the editing of a target DNA. As used herein, the PAM is a nucleotide sequence proximal to the protospacer that, in conjunction with the targeting sequence of the gNA, helps the orientation and positioning of the CasX for the potential cleavage of the protospacer strand(s). Herein, the protospacer is defined as the DNA sequence complementary to the targeting sequence of the guide RNA and the DNA complementary to that sequence, referred to as the target strand and non-target strand, respectively. PAM sequences may be degenerate, and specific RNP constructs may have different preferred and tolerated PAM sequences that support different efficiencies of cleavage. Following convention, unless stated otherwise, the disclosure refers to both the PAM and the protospacer sequence and their directionality according to the orientation of the non-target strand. This does not imply that the PAM sequence of the non-target strand, rather than the target strand, is determinative of cleavage or mechanistically involved in target recognition. For example, when reference is to a TTC PAM, it may in fact be the complementary GAA sequence that is required for target cleavage, or it may be some combination of nucleotides from both strands. In the case of the CasX proteins disclosed herein, the PAM is located 5′ of the protospacer with a single nucleotide separating the PAM from the first nucleotide of the protospacer. Thus, in the case of reference CasX, a TTC PAM should be understood to mean a sequence following the formula 5′- . . . NNTTCN(protospacer)NNNNNN . . . 3′ (SEQ ID NO: 247) where ‘N’ is any DNA nucleotide and ‘(protospacer)’ is a DNA sequence having identity with the targeting sequence of the guide RNA. In the case of a CasX variant with expanded PAM recognition, a TTC, CTC, GTC, or ATC PAM should be understood to mean a sequence following the formulae: 5′- . . . NNTTCN(protospacer)NNNNNN . . . 3′ (SEQ ID NO: 247); 5′- . . . NNCTCN(protospacer)NNNNNN . . . 3′ (SEQ ID NO: 248); 5′- . . . NNGTCN(protospacer)NNNNNN . . . 3′ (SEQ ID NO: 249); or 5′- . . . NNATCN(protospacer)NNNNNN . . . 3′ (SEQ ID NO: 250). Alternatively, a TC PAM should be understood to mean a sequence following the formula 5′- . . . NNNTCN(protospacer)NNNNNN . . . 3′ (SEQ ID NO: 251). In some embodiments, a CasX variant has improved editing of a PAM sequence exhibits greater editing efficiency and/or binding of a target sequence in the target DNA when any one of the PAM sequences TTC, ATC, GTC, or CTC is located 1 nucleotide 5′ to the non-target strand of the protospacer having identity with the targeting sequence of the gNA in a cellular assay system compared to the editing efficiency and/or binding of an RNP comprising a reference CasX protein in a comparable assay system. In some embodiments, the PAM sequence is TTC. In some embodiments, the PAM sequence is ATC. In some embodiments, the PAM sequence is CTC. In some embodiments, the PAM sequence is GTC.

In some embodiments, the variant is a CRISPR associated protein, wherein the variant has one or more altered activities compared to a reference. For example, in some embodiments, the variant has altered target specificity, for example specificity for RNA instead of DNA, compared to a reference. In some embodiments, the variant is a nickase Cas protein, or a dead Cas protein, compared to a reference protein which cleaves double stranded DNA.

In some embodiments, wherein the variant is a CasX variant, the one or more improved characteristics are improved compared to a reference CasX of SEQ ID NO: 1. In other embodiments, wherein the variant is a CasX variant, the one or more improved characteristics are improved compared to a reference CasX of SEQ ID NO: 2. In still further embodiments, wherein the variant is a CasX variant, the one or more improved characteristics are improved compared to a reference CasX of SEQ ID NO: 3.

In some embodiments, the CasX variant protein has least 60% identity, at least 70% identity, at least 80% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, at least 99.5% identity, at least 99.6% identity, at least 99.7% identity, at least 99.8% identity or at least 99.9% identity to one of SEQ ID NO: 1, SEQ ID NO: 2, or SEQ ID NO: 3. In some embodiments, the CasX variant protein comprises or consists of a sequence that has at least 1, at least 2, at least 3, at least 4, at least 5, at least 6, at least 7, at least 8, at least 9, at least 10, at least 20, at least 30, at least 40 or at least 50 mutations relative to the sequence of SEQ ID NO: 1, SEQ ID NO: 2, or SEQ ID NO: 3. These mutations can be insertions, deletions, amino acid substitutions, or any combinations thereof.

In some embodiments, the CasX variant protein has sequence identity to SEQ ID NO: 2 or a portion thereof.

In some embodiments of the CasX variants described herein, the at least one modification comprises: (a) a substitution of 1 to 100 consecutive or non-consecutive amino acids in the CasX variant; (b) a deletion of 1 to 100 consecutive or non-consecutive amino acids in the CasX variant; (c) an insertion of 1 to 100 consecutive or non-consecutive amino acids in the CasX; or (d) any combination of (a)-(c). In some embodiments, the at least one modification comprises: (a) a substitution of 5-10 consecutive or non-consecutive amino acids in the CasX variant; (b) a deletion of 1-5 consecutive or non-consecutive amino acids in the CasX variant; (c) an insertion of 1-5 consecutive or non-consecutive amino acids in the CasX; or (d) any combination of (a)-(c).

In some embodiments, the CasX variant protein comprises a substitution of Y789T of SEQ ID NO: 2, a deletion of P793 of SEQ ID NO: 2, a substitution of Y789D of SEQ ID NO: 2, a substitution of T72S of SEQ ID NO: 2, a substitution of I546V of SEQ ID NO: 2, a substitution of E552A of SEQ ID NO: 2, a substitution of A636D of SEQ ID NO: 2, a substitution of F536S of SEQ ID NO:2, a substitution of A708K of SEQ ID NO: 2, a substitution of Y797L of SEQ ID NO: 2, a substitution of L792G SEQ ID NO: 2, a substitution of A739V of SEQ ID NO: 2, a substitution of G791M of SEQ ID NO: 2, a insertion of A at position 661 (\hat{ }G661A) of SEQ ID NO: 2, a substitution of A788W of SEQ ID NO: 2, a substitution of K390R of SEQ ID NO: 2, a substitution of A751S of SEQ ID NO: 2, a substitution of E385A of SEQ ID NO: 2, an insertion of P at position 696 of SEQ ID NO: 2, an insertion of M at position 773 of SEQ ID NO: 2, a substitution of G695H of SEQ ID NO: 2, an insertion of AS at position 793 of SEQ ID NO: 2, an insertion of AS at position 795 of SEQ ID NO: 2, a substitution of C477R of SEQ ID NO: 2, a substitution of C477K of SEQ ID NO: 2, a substitution of C479A of SEQ ID NO: 2, a substitution of C479L of SEQ ID NO: 2, a substitution of I55F of SEQ ID NO: 2, a substitution of K210R of SEQ ID NO: 2, a substitution of C233S of SEQ ID NO: 2, a substitution of D231N of SEQ ID NO: 2, a substitution of Q338E of SEQ ID NO: 2, a substitution of Q338R of SEQ ID NO: 2, a substitution of L379R of SEQ ID NO: 2, a substitution of K390R of SEQ ID NO: 2, a substitution of L481Q of SEQ ID NO: 2, a substitution of F495S of SEQ ID NO:2, a substitution of D600N of SEQ ID NO: 2, a substitution of T886K of SEQ ID NO: 2, a substitution of A739V of SEQ ID NO: 2, a substitution of K460N of SEQ ID NO: 2, a substitution of I199F of SEQ ID NO: 2, a substitution of G492P of SEQ ID NO: 2, a substitution of T1531 of SEQ ID NO: 2, a substitution of R591I of SEQ ID NO: 2, an insertion of AS at position 795 of SEQ ID NO: 2, an insertion of AS at position 796 of SEQ ID NO:2, an insertion of L at position 889 of SEQ ID NO: 2, a substitution of E121D of SEQ ID NO: 2, a substitution of S270W of SEQ ID NO: 2, a substitution of E712Q of SEQ ID NO: 2, a substitution of K942Q of SEQ ID NO: 2, a substitution of E552K of SEQ ID NO:2, a substitution of K25Q of SEQ ID NO: 2, a substitution of N47D of SEQ ID NO: 2, an insertion of T at position 696 of SEQ ID NO: 2, a substitution of L685I of SEQ ID NO: 2, a substitution of N880D of SEQ ID NO: 2, a substitution of Q102R of SEQ ID NO: 2, a substitution of M734K of SEQ ID NO: 2, a substitution of A724S of SEQ ID NO: 2, a substitution of T704K of SEQ ID NO: 2, a substitution of P224K of SEQ ID NO: 2, a substitution of 1(25R of SEQ ID NO: 2, a substitution of M29E of SEQ ID NO: 2, a substitution of H152D of SEQ ID NO: 2, a substitution of S219R of SEQ ID NO: 2, a substitution of E475K of SEQ ID NO: 2, a substitution of G226R of SEQ ID NO: 2, a substitution of A377K of SEQ ID NO: 2, a substitution of E480K of SEQ ID NO: 2, a substitution of K416E of SEQ ID NO: 2, a substitution of H164R of SEQ ID NO: 2, a substitution of K767R of SEQ ID NO: 2, a substitution of I7F of SEQ ID NO: 2, a substitution of M29R of SEQ ID NO: 2, a substitution of H435R of SEQ ID NO: 2, a substitution of E385Q of SEQ ID NO: 2, a substitution of E385K of SEQ ID NO: 2, a substitution of I279F of SEQ ID NO: 2, a substitution of D489S of SEQ ID NO: 2, a substitution of D732N of SEQ ID NO: 2, a substitution of A739T of SEQ ID NO: 2, a substitution of W885R of SEQ ID NO: 2, a substitution of E53K of SEQ ID NO: 2, a substitution of A238T of SEQ ID NO: 2, a substitution of P283Q of SEQ ID NO: 2, a substitution of E292K of SEQ ID NO: 2, a substitution of Q628E of SEQ ID NO: 2, a substitution of R388Q of SEQ ID NO: 2, a substitution of G791M of SEQ ID NO: 2, a substitution of L792K of SEQ ID NO: 2, a substitution of L792E of SEQ ID NO: 2, a substitution of M779N of SEQ ID NO: 2, a substitution of G27D of SEQ ID NO: 2, a substitution of K955R of SEQ ID NO: 2, a substitution of S867R of SEQ ID NO: 2, a substitution of R693I of SEQ ID NO: 2, a substitution of F189Y of SEQ ID NO: 2, a substitution of V635M of SEQ ID NO: 2, a substitution of F399L of SEQ ID NO: 2, a substitution of E498K of SEQ ID NO: 2, a substitution of E386R of SEQ ID NO: 2, a substitution of V254G of SEQ ID NO: 2, a substitution of P793S of SEQ ID NO: 2, a substitution of K188E of SEQ ID NO: 2, a substitution of QT945KI of SEQ ID NO: 2, a substitution of T620P of SEQ ID NO: 2, a substitution of T946P of SEQ ID NO: 2, a substitution of TT949PP of SEQ ID NO: 2, a substitution of N952T of SEQ ID NO: 2, a substitution of K682E of SEQ ID NO: 2, a substitution of K975R of SEQ ID NO: 2, a substitution of L212P of SEQ ID NO: 2, a substitution of E292R of SEQ ID NO: 2, a substitution of 1303K of SEQ ID NO: 2, a substitution of C349E of SEQ ID NO: 2, a substitution of E385P of SEQ ID NO: 2, a substitution of E386N of SEQ ID NO: 2, a substitution of D387K of SEQ ID NO: 2, a substitution of L404K of SEQ ID NO: 2, a substitution of E466H of SEQ ID NO: 2, a substitution of C477Q of SEQ ID NO: 2, a substitution of C477H of SEQ ID NO: 2, a substitution of C479A of SEQ ID NO: 2, a substitution of D659H of SEQ ID NO: 2, a substitution of T806V of SEQ ID NO: 2, a substitution of K808S of SEQ ID NO: 2, an insertion of AS at position 797 of SEQ ID NO: 2, a substitution of V959M of SEQ ID NO: 2, a substitution of K975Q of SEQ ID NO: 2, a substitution of W974G of SEQ ID NO: 2, a substitution of A708Q of SEQ ID NO: 2, a substitution of V711K of SEQ ID NO: 2, a substitution of D733T of SEQ ID NO: 2, a substitution of L742W of SEQ ID NO: 2, a substitution of V747K of SEQ ID NO: 2, a substitution of F755M of SEQ ID NO: 2, a substitution of M771A of SEQ ID NO: 2, a substitution of M771Q of SEQ ID NO: 2, a substitution of W782Q of SEQ ID NO: 2, a substitution of G791F, of SEQ ID NO: 2 a substitution of L792D of SEQ ID NO: 2, a substitution of L792K of SEQ ID NO: 2, a substitution of P793Q of SEQ ID NO: 2, a substitution of P793G of SEQ ID NO: 2, a substitution of Q804A of SEQ ID NO: 2, a substitution of Y966N of SEQ ID NO: 2, a substitution of Y723N of SEQ ID NO: 2, a substitution of Y857R of SEQ ID NO: 2, a substitution of S890R of SEQ ID NO: 2, a substitution of S932M of SEQ ID NO: 2, a substitution of L897M of SEQ ID NO: 2, a substitution of R624G of SEQ ID NO: 2, a substitution of 5603G of SEQ ID NO: 2, a substitution of N737S of SEQ ID NO: 2, a substitution of L307K of SEQ ID NO: 2, a substitution of I658V of SEQ ID NO: 2, an insertion of PT at position 688 of SEQ ID NO: 2, an insertion of SA at position 794 of SEQ ID NO: 2, a substitution of S877R of SEQ ID NO: 2, a substitution of N580T of SEQ ID NO: 2, a substitution of V335G of SEQ ID NO: 2, a substitution of T620S of SEQ ID NO: 2, a substitution of W345G of SEQ ID NO: 2, a substitution of T280S of SEQ ID NO: 2, a substitution of L406P of SEQ ID NO: 2, a substitution of A612D of SEQ ID NO: 2, a substitution of A75I S of SEQ ID NO: 2, a substitution of E386R of SEQ ID NO: 2, a substitution of V351M of SEQ ID NO: 2, a substitution of K210N of SEQ ID NO: 2, a substitution of D40A of SEQ ID NO: 2, a substitution of E773G of SEQ ID NO: 2, a substitution of H207L of SEQ ID NO: 2, a substitution of T62A SEQ ID NO: 2, a substitution of T287P of SEQ ID NO: 2, a substitution of T832A of SEQ ID NO: 2, a substitution of A893S of SEQ ID NO: 2, an insertion of V at position 14 of SEQ ID NO: 2, an insertion of AG at position 13 of SEQ ID NO: 2, a substitution of R11V of SEQ ID NO: 2, a substitution of R12N of SEQ ID NO: 2, a substitution of R13H of SEQ ID NO: 2, an insertion of Y at position 13 of SEQ ID NO: 2, a substitution of R12L of SEQ ID NO: 2, an insertion of Q at position 13 of SEQ ID NO: 2, an substitution of V15S of SEQ ID NO: 2, an insertion of D at position 17 of SEQ ID NO: 2, or a combination thereof.

In some embodiments, a CasX variant protein comprises more than one substitution, insertion and/or deletion of a reference CasX protein amino acid sequence. In some embodiments, the reference CasX protein comprises or consists essentially of SEQ ID NO: 2. In some embodiments, a CasX variant protein comprises a substitution of S794R and a substitution of Y797L of SEQ ID NO: 2. In some embodiments, a CasX variant protein comprises a substitution of K416E and a substitution of A708K of SEQ ID NO: 2. In some embodiments, a CasX variant comprises a substitution of A708K and a deletion of P793 of SEQ ID NO: 2. In some embodiments, a CasX variant protein comprises a deletion of P793 and a substitution of P793AS SEQ ID NO: 2. In some embodiments, a CasX variant protein comprises a substitution of Q367K and a substitution of I425S of SEQ ID NO: 2. In some embodiments, a CasX variant protein comprises a substitution of A708K, a deletion of P position 793 and a substitution A793V of SEQ ID NO: 2. In some embodiments, a CasX variant protein comprises a substitution of Q338R and a substitution of A339E of SEQ ID NO: 2. In some embodiments, a CasX variant protein comprises a substitution of Q338R and a substitution of A339K of SEQ ID NO: 2. In some embodiments, a CasX variant protein comprises a substitution of S507G and a substitution of G508R of SEQ ID NO: 2. In some embodiments, a CasX variant protein comprises a substitution of L379R, a substitution of A708K and a deletion of P at position 793 of SEQ ID NO: 2. In some embodiments, a CasX variant protein comprises a substitution of C477K, a substitution of A708K and a deletion of P at position 793 of SEQ ID NO: 2. In some embodiments, a CasX variant protein comprises a substitution of L379R, a substitution of C477K, a substitution of A708K and a deletion of P at position of 793 of SEQ ID NO: 2. In some embodiments, a CasX variant protein comprises a substitution of L379R, a substitution of A708K, a deletion of P at position 793 and a substitution A739V of SEQ ID NO: 2. In some embodiments, a CasX variant protein comprises a substitution of C477K, a substitution of A708K, a deletion of P at position 793 and a substitution of A739V of SEQ ID NO: 2. In some embodiments, a CasX variant protein comprises a substitution of L379R, a substitution of C477K, a substitution of A708K, a deletion of P at position 793 and a substitution of A739V of SEQ ID NO: 2. In some embodiments, a CasX variant protein comprises a substitution of L379R, a substitution of A708K, a deletion of P at position 793 and a substitution of M779N of SEQ ID NO: 2. In some embodiments, a CasX variant protein comprises a substitution of L379R, a substitution of A708K, a deletion of P at position 793 and a substitution of M771N of SEQ ID NO: 2. In some embodiments, a CasX variant protein comprises a substitution of L379R, a substitution of 708K, a deletion of P at position 793 and a substitution of D489S of SEQ ID NO: 2. In some embodiments, a CasX variant protein comprises a substitution of L379R, a substitution of A708K, a deletion of P at position 793 and a substitution of A739T of SEQ ID NO: 2. In some embodiments, a CasX variant protein comprises a substitution of L379R, a substitution of A708K, a deletion of P at position 793 and a substitution of D732N of SEQ ID NO: 2. In some embodiments, a CasX variant protein comprises a substitution of L379R, a substitution of A708K, a deletion of P at position 793 and a substitution of G791M of SEQ ID NO: 2. In some embodiments, a CasX variant protein comprises a substitution of L379R, a substitution of 708K, a deletion of P at position 793 and a substitution of Y797L of SEQ ID NO: 2. In some embodiments, a CasX variant protein comprises a substitution of L379R, a substitution of C477K, a substitution of A708K, a deletion of P at position 793 and a substitution of M779N of SEQ ID NO: 2. In some embodiments, a CasX variant protein comprises a substitution of L379R, a substitution of C477K, a substitution of A708K, a deletion of P at position 793 and a substitution of M771N of SEQ ID NO: 2. In some embodiments, a CasX variant protein comprises a substitution of L379R, a substitution of C477K, a substitution of A708K, a deletion of P at position 793 and a substitution of D489S of SEQ ID NO: 2. In some embodiments, a CasX variant protein comprises a substitution of L379R, a substitution of C477K, a substitution of A708K, a deletion of P at position 793 and a substitution of A739T of SEQ ID NO: 2. In some embodiments, a CasX variant protein comprises a substitution of L379R, a substitution of C477K, a substitution of A708K, a deletion of P at position 793 and a substitution of D732N of SEQ ID NO: 2. In some embodiments, a CasX variant protein comprises a substitution of L379R, a substitution of C477K, a substitution of A708K, a deletion of P at position 793 and a substitution of G791M of SEQ ID NO: 2. In some embodiments, a CasX variant protein comprises a substitution of L379R, a substitution of C477K, a substitution of A708K, a deletion of P at position 793 and a substitution of Y797L of SEQ ID NO: 2. In some embodiments, a CasX variant protein comprises a substitution of L379R, a substitution of C477K, a substitution of A708K, a deletion of P at position 793 and a substitution of T620P of SEQ ID NO: 2. In some embodiments, a CasX variant protein comprises a substitution of A708K, a deletion of P at position 793 and a substitution of E386S of SEQ ID NO: 2. In some embodiments, a CasX variant protein comprises a substitution of E386R, a substitution of F399L and a deletion of P at position 793 of SEQ ID NO: 2. In some embodiments, a CasX variant protein comprises a substitution of R581I and A739V of SEQ ID NO: 2.

In some embodiments, a CasX variant protein comprises more than one substitution, insertion and/or deletion of a reference CasX protein amino acid sequence. In some embodiments, the reference CasX protein comprises or consists essentially of SEQ ID NO: 2. In some embodiments, a CasX variant protein comprises a substitution of S794R and a substitution of Y797L of SEQ ID NO: 2. In some embodiments, a CasX variant protein comprises a substitution of K416E and a substitution of A708K of SEQ ID NO: 2. In some embodiments, a CasX variant protein comprises a substitution of A708K and a deletion of P793 of SEQ ID NO: 2. In some embodiments, a CasX variant protein comprises a deletion of P793 and an insertion of AS at position 795 SEQ ID NO: 2. In some embodiments, a CasX variant protein comprises a substitution of Q367K and a substitution of I425S of SEQ ID NO: 2. In some embodiments, a CasX variant protein comprises a substitution of A708K, a deletion of P position 793 and a substitution A793V of SEQ ID NO: 2. In some embodiments, a CasX variant protein comprises a substitution of Q338R and a substitution of A339E of SEQ ID NO: 2. In some embodiments, a CasX variant protein comprises a substitution of Q338R and a substitution of A339K of SEQ ID NO: 2. In some embodiments, a CasX variant protein comprises a substitution of S507G and a substitution of G508R of SEQ ID NO: 2. In some embodiments, a CasX variant protein comprises a substitution of L379R, a substitution of A708K and a deletion of P at position 793 of SEQ ID NO: 2. In some embodiments, a CasX variant protein comprises a substitution of C477K, a substitution of A708K and a deletion of P at position 793 of SEQ ID NO: 2. In some embodiments, a CasX variant protein comprises a substitution of L379R, a substitution of C477K, a substitution of A708K and a deletion of P at position of 793 of SEQ ID NO: 2. In some embodiments, a CasX variant protein comprises a substitution of L379R, a substitution of A708K, a deletion of P at position 793 and a substitution A739V of SEQ ID NO: 2. In some embodiments, a CasX variant protein comprises a substitution of C477K, a substitution of A708K, a deletion of P at position 793 and a substitution of A739V of SEQ ID NO: 2. In some embodiments, a CasX variant protein comprises a substitution of L379R, a substitution of C477K, a substitution of A708K, a deletion of P at position 793 and a substitution of A739V of SEQ ID NO: 2. In some embodiments, a CasX variant protein comprises a substitution of L379R, a substitution of A708K, a deletion of P at position 793 and a substitution of M779N of SEQ ID NO: 2. In some embodiments, a CasX variant protein comprises a substitution of L379R, a substitution of A708K, a deletion of P at position 793 and a substitution of M771N of SEQ ID NO: 2. In some embodiments, a CasX variant protein comprises a substitution of L379R, a substitution of 708K, a deletion of P at position 793 and a substitution of D489S of SEQ ID NO: 2. In some embodiments, a CasX variant protein comprises a substitution of L379R, a substitution of A708K, a deletion of P at position 793 and a substitution of A739T of SEQ ID NO: 2. In some embodiments, a CasX variant protein comprises a substitution of L379R, a substitution of A708K, a deletion of P at position 793 and a substitution of D732N of SEQ ID NO: 2. In some embodiments, a CasX variant protein comprises a substitution of L379R, a substitution of A708K, a deletion of P at position 793 and a substitution of G791M of SEQ ID NO: 2. In some embodiments, a CasX variant protein comprises a substitution of L379R, a substitution of 708K, a deletion of P at position 793 and a substitution of Y797L of SEQ ID NO: 2. In some embodiments, a CasX variant protein comprises a substitution of L379R, a substitution of C477K, a substitution of A708K, a deletion of P at position 793 and a substitution of M779N of SEQ ID NO: 2. In some embodiments, a CasX variant protein comprises a substitution of L379R, a substitution of C477K, a substitution of A708K, a deletion of P at position 793 and a substitution of M771N of SEQ ID NO: 2. In some embodiments, a CasX variant protein comprises a substitution of L379R, a substitution of C477K, a substitution of A708K, a deletion of P at position 793 and a substitution of D489S of SEQ ID NO: 2. In some embodiments, a CasX variant protein comprises a substitution of L379R, a substitution of C477K, a substitution of A708K, a deletion of P at position 793 and a substitution of A739T of SEQ ID NO: 2. In some embodiments, a CasX variant protein comprises a substitution of L379R, a substitution of C477K, a substitution of A708K, a deletion of P at position 793 and a substitution of D732N of SEQ ID NO: 2. In some embodiments, a CasX variant protein comprises a substitution of L379R, a substitution of C477K, a substitution of A708K, a deletion of P at position 793 and a substitution of G791M of SEQ ID NO: 2. In some embodiments, a CasX variant protein comprises a substitution of L379R, a substitution of C477K, a substitution of A708K, a deletion of P at position 793 and a substitution of Y797L of SEQ ID NO: 2. In some embodiments, a CasX variant protein comprises a substitution of L379R, a substitution of C477K, a substitution of A708K, a deletion of P at position 793 and a substitution of T620P of SEQ ID NO: 2. In some embodiments, a CasX variant protein comprises a substitution of A708K, a deletion of P at position 793 and a substitution of E386S of SEQ ID NO: 2. In some embodiments, a CasX variant protein comprises a substitution of E386R, a substitution of F399L and a deletion of P at position 793 of SEQ ID NO: 2. In some embodiments, a CasX variant protein comprises a substitution of R581I and A739V of SEQ ID NO: 2. In some embodiments, a CasX variant comprises any combination of the foregoing embodiments of this paragraph.

In some embodiments, a CasX variant protein comprises more than one substitution, insertion and/or deletion of a reference CasX protein amino acid sequence. In some embodiments, a CasX variant protein comprises a substitution of A708K, a deletion of P at position 793 and a substitution of A739V of SEQ ID NO: 2. In some embodiments, a CasX variant protein comprises a substitution of L379R, a substitution of A708K and a deletion of P at position 793 of SEQ ID NO: 2. In some embodiments, a CasX variant protein comprises a substitution of C477K, a substitution of A708K and a deletion of P at position 793 of SEQ ID NO: 2. In some embodiments, a CasX variant protein comprises a substitution of L379R, a substitution of C477K, a substitution of A708K and a deletion of P at position 793 of SEQ ID NO: 2. In some embodiments, a CasX variant protein comprises a substitution of L379R, a substitution of A708K, a deletion of P at position 793 and a substitution of A739V of SEQ ID NO: 2. In some embodiments, a CasX variant protein comprises a substitution of C477K, a substitution of A708K, a deletion of P at position 793 and a substitution of A739 of SEQ ID NO: 2. In some embodiments, a CasX variant protein comprises a substitution of L379R, a substitution of C477K, a substitution of A708K, a deletion of P at position 793 and a substitution of A739V of SEQ ID NO: 2. In some embodiments, a CasX variant protein comprises a substitution of L379R, a substitution of C477K, a substitution of A708K, a deletion of P at position 793 and a substitution of T620P of SEQ ID NO: 2. In some embodiments, a CasX variant protein comprises a substitution of M771A of SEQ ID NO: 2. In some embodiments, a CasX variant protein comprises a substitution of L379R, a substitution of A708K, a deletion of P at position 793 and a substitution of D732N of SEQ ID NO: 2. In some embodiments, a CasX variant comprises any combination of the foregoing embodiments of this paragraph.

In some embodiments, a CasX variant protein comprises a substitution of W782Q of SEQ ID NO: 2. In some embodiments, a CasX variant protein comprises a substitution of M771Q of SEQ ID NO: 2. In some embodiments, a CasX variant protein comprises a substitution of R458I and a substitution of A739V of SEQ ID NO: 2. In some embodiments, a CasX variant protein comprises a substitution of L379R, a substitution of A708K, a deletion of P at position 793 and a substitution of M771N of SEQ ID NO: 2. In some embodiments, a CasX variant protein comprises a substitution of L379R, a substitution of A708K, a deletion of P at position 793 and a substitution of A739T of SEQ ID NO: 2. In some embodiments, a CasX variant protein comprises a substitution of L379R, a substitution of C477K, a substitution of A708K, a deletion of P at position 793 and a substitution of D489S of SEQ ID NO: 2. In some embodiments, a CasX variant protein comprises a substitution of L379R, a substitution of C477K, a substitution of A708K, a deletion of P at position 793 and a substitution of D732N of SEQ ID NO: 2. In some embodiments, a CasX variant protein comprises a substitution of V711K of SEQ ID NO: 2. In some embodiments, a CasX variant protein comprises a substitution of L379R, a substitution of C477K, a substitution of A708K, a deletion of P at position 793 and a substitution of Y797L of SEQ ID NO: 2. In some embodiments, a CasX variant protein comprises a substitution of L379R, a substitution of A708K and a deletion of P at position 793 of SEQ ID NO: 2. In some embodiments, a CasX variant protein comprises a substitution of L379R, a substitution of C477K, a substitution of A708K, a deletion of P at position 793 and a substitution of M771N of SEQ ID NO: 2. In some embodiments, a CasX variant protein comprises a substitution of A708K, a substitution of P at position 793 and a substitution of E386S of SEQ ID NO: 2. In some embodiments, a CasX variant protein comprises a substitution of L379R, a substitution of C477K, a substitution of A708K and a deletion of P at position 793 of SEQ ID NO: 2. In some embodiments, a CasX variant protein comprises a substitution of L792D of SEQ ID NO: 2. In some embodiments, a CasX variant protein comprises a substitution of G791F of SEQ ID NO: 2. In some embodiments, a CasX variant protein comprises a substitution of A708K, a deletion of P at position 793 and a substitution of A739V of SEQ ID NO: 2. In some embodiments, a CasX variant protein comprises a substitution of L379R, a substitution of A708K, a deletion of P at position 793 and a substitution of A739V of SEQ ID NO: 2. In some embodiments, a CasX variant protein comprises a substitution of C477K, a substitution of A708K and a substitution of P at position 793 of SEQ ID NO: 2. In some embodiments, a CasX variant protein comprises a substitution of L249I and a substitution of M771N of SEQ ID NO: 2. In some embodiments, a CasX variant protein comprises a substitution of V747K of SEQ ID NO: 2. In some embodiments, a CasX variant protein comprises a substitution of L379R, a substitution of C477, a substitution of A708K, a deletion of P at position 793 and a substitution of M779N of SEQ ID NO: 2. In some embodiments, a CasX variant protein comprises a substitution of F755M. In some embodiments, a CasX variant comprises any combination of the foregoing embodiments of this paragraph.

In some embodiments, the CasX variant comprises at least one modification in the NTSB domain.

In some embodiments, the CasX variant comprises at least one modification in the TSL domain. In some embodiments, the at least one modification in the TSL domain comprises an amino acid substitution of one or more of amino acids Y857, S890, or S932 of SEQ ID NO: 2.

In some embodiments, the CasX variant comprises at least one modification in the helical I domain. In some embodiments, the at least one modification in the helical I domain comprises an amino acid substitution of one or more of amino acids S219, L249, E259, Q252, E292, L307, or D318 of SEQ ID NO: 2.

In some embodiments, the CasX variant comprises at least one modification in the helical II domain. In some embodiments, the at least one modification in the helical II domain comprises an amino acid substitution of one or more of amino acids D361, L379, E385, E386, D387, F399, L404, R458, C477, or D489 of SEQ ID NO: 2.

In some embodiments, the CasX variant comprises at least one modification in the OBD domain. In some embodiments, the at least one modification in the OBD comprises an amino acid substitution of one or more of amino acids F536, E552, T620, or 1658 of SEQ ID NO: 2.

In some embodiments, the CasX variant comprises at least one modification in the RuvC DNA cleavage domain. In some embodiments, the at least one modification in the RuvC DNA cleavage domain comprises an amino acid substitution of one or more of amino acids K682, G695, A708, V711, D732, A739, D733, L742, V747, F755, M771, M779, W782, A788, G791, L792, P793, Y797, M799, Q804, 5819, or Y857 or a deletion of amino acid P793 of SEQ ID NO: 2.

In some embodiments, a CasX variant protein comprises at least one modification compared to the reference CasX sequence of SEQ ID NO:2, wherein the at least one modification is selected from one or more of: an amino acid substitution of L379R; an amino acid substitution of A708K; an amino acid substitution of T620P; an amino acid substitution of E385P; an amino acid substitution of Y857R; an amino acid substitution of I658V; an amino acid substitution of F399L; an amino acid substitution of Q252K; an amino acid substitution of L404K; and an amino acid deletion of [P793]. In another embodiment, a CasX variant protein comprises any combination of the foregoing substitutions or deletions compared to the reference CasX sequence of SEQ ID NO:2. In another embodiment, the CasX variant protein can, in addition to the foregoing substitutions or deletions, further comprise a substitution of an NTSB and/or a helical 1b domain from the reference CasX of SEQ ID NO:1.

In some embodiments, a CasX variant protein comprises a sequence set forth in Table 1. In other embodiments, a CasX variant protein comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence set forth in Table 1. In other embodiments, a CasX variant protein comprises a sequence set forth in Table 1, and further comprises one or more NLS disclosed herein on either the N-terminus, the C-terminus, or both. It will be understood that in some cases, the N-terminal methionine of the CasX variants of the Table is removed from the expressed CasX variant during post-translational modification.

TABLE 1
CasX Variant Sequences

| Description* | SEQ ID NO |
|---|---|
| TSL, Helical I, Helical II, OBD and RuvC domains from SEQ ID NO: 2 | 252 |
| and an NTSB domain from SEQ ID NO: 1 | |
| NTSB, Helical I, Helical II, OBD and RuvC domains from SEQ ID NO: 2 | 253 |
| and a TSL domain from SEQ ID NO: 1. | |
| TSL, Helical I, Helical II, OBD and RuvC domains from SEQ ID NO: 1 | 254 |
| and an NTSB domain from SEQ ID NO: 2 | |
| NTSB, Helical I, Helical II, OBD and RuvC domains from SEQ ID NO: 1 | 255 |
| and an TSL domain from SEQ ID NO: 2. | |
| NTSB, TSL, Helical I, Helical II and OBD domains SEQ ID NO: 2 and an | 256 |
| exogenous RuvC domain or a portion thereof from a second CasX protein. | |
| No description | 257 |
| NTSB, TSL, Helical II, OBD and RuvC domains from SEQ ID NO: 2 and | 258 |
| a Helical I domain from SEQ ID NO: 1 | |
| NTSB, TSL, Helical I, OBD and RuvC domains from SEQ ID NO: 2 and a | 259 |
| Helical II domain from SEQ ID NO: 1 | |
| NTSB, TSL, Helical I, Helical II and RuvC domains from a first CasX | 260 |
| protein and an exogenous OBD or a part thereof from a second CasX protein | |
| No description | 261 |
| No description | 262 |
| substitution of L379R, a substitution of C477K, a substitution of A708K, a | 263 |
| deletion of P at position 793 and a substitution of T620P of SEQ ID NO: 2 | |
| substitution of M771A of SEQ ID NO: 2. | 264 |
| substitution of L379R, a substitution of A708K, a deletion of P at position | 265 |
| 793 and a substitution of D732N of SEQ ID NO: 2. | |
| substitution of W782Q of SEQ ID NO: 2. | 266 |
| substitution of M771Q of SEQ ID NO: 2 | 267 |
| substitution of R458I and a substitution of A739V of SEQ ID NO: 2. | 268 |
| L379R, a substitution of A708K, a deletion of P at position 793 and a | 269 |
| substitution of M771N of SEQ ID NO: 2 | |
| substitution of L379R, a substitution of A708K, a deletion of P at position | 270 |
| 793 and a substitution of A739T of SEQ ID NO: 2 | |
| substitution of L379R, a substitution of C477K, a substitution of A708K, a | 271 |
| deletion of P at position 793 and a substitution of D489S of SEQ ID NO: 2. | |
| substitution of L379R, a substitution of C477K, a substitution of A708K, a | 272 |
| deletion of P at position 793 and a substitution of D732N of SEQ ID NO: 2. | |
| substitution of V711K of SEQ ID NO: 2. | 273 |
| substitution of L379R, a substitution of C477K, a substitution of A708K, a | 274 |
| deletion of P at position 793 and a substitution of Y797L of SEQ ID NO: 2. | |
| 119, substitution of L379R, a substitution of A708K and a deletion of P at | 275 |
| position 793 of SEQ ID NO: 2. | |
| substitution of L379R, a substitution of C477K, a substitution of A708K, a | 276 |
| deletion of P at position 793 and a substitution of M771N of SEQ ID NO: 2. | |
| substitution of A708K, a deletion of P at position 793 and a substitution of | 277 |
| E386S of SEQ ID NO: 2. | |
| substitution of L379R, a substitution of C477K, a substitution of A708K | 278 |
| and a deletion of P at position 793 of SEQ ID NO: 2. | |
| substitution of L792D of SEQ ID NO: 2. | 279 |
| substitution of G791F of SEQ ID NO: 2. | 280 |
| substitution of A708K, a deletion of P at position 793 and a substitution of | 281 |
| A739V of SEQ ID NO: 2. | |
| substitution of L379R, a substitution of A708K, a deletion of P at position | 282 |
| 793 and a substitution of A739V of SEQ ID NO: 2. | |
| substitution of C477K, a substitution of A708K and a deletion of P at | 283 |
| position 793 of SEQ ID NO: 2. | |
| substitution of L249I and a substitution of M771N of SEQ ID NO: 2. | 284 |
| substitution of V747K of SEQ ID NO: 2. | 285 |
| substitution of L379R, a substitution of C477K, a substitution of A708K, a | 286 |
| deletion of P at position 793 and a substitution of M779N of SEQ ID NO: 2. | |
| L379R, F755M | 287 |
| 429, L379R, A708K, P793_, Y857R | 288 |
| 430, L379R, A708K, P793_, Y857R, I658V | 289 |
| 431, L379R, A708K, P793_, Y857R, I658V, E386N | 290 |
| 432, L379R, A708K, P793_, Y857R, I658V, L404K | 291 |
| 433, L379R, A708K, P793_, Y857R, I658V, \hat{ }V192 | 292 |
| 434, L379R, A708K, P793_, Y857R, I658V, L404K, E386N | 293 |
| 435, L379R, A708K, P793_, Y857R, I658V, F399L | 294 |
| 436, L379R, A708K, P793_, Y857R, I658V, F399L, E386N | 295 |
| 437, L379R, A708K, P793_, Y857R, I658V, F399L, C477S | 296 |
| 438, L379R, A708K, P793_, Y857R, I658V, F399L, L404K | 297 |
| 439, L379R, A708K, P793_, Y857R, I658V, F399L, E386N, C477S, L404K | 298 |
| 440, L379R, A708K, P793_, Y857R, I658V, F399L, Y797L | 299 |
| 441, L379R, A708K, P793_, Y857R, I658V, F399L, Y797L, E386N | 300 |
| 442, L379R, A708K, P793_, Y857R, I658V, F399L, Y797L, E386N, | 301 |
| C477S, L404K | |
| 443, L379R, A708K, P793_, Y857R, I658V, Y797L | 302 |
| 444, L379R, A708K, P793_, Y857R, I658V, Y797L, L404K | 303 |
| 445, L379R, A708K, P793_, Y857R, I658V, Y797L, E386N | 304 |
| 446, L379R, A708K, P793_, Y857R, I658V, Y797L, E386N, C477S, L404K | 305 |
| 447, L379R, A708K, P793_, Y857R, E386N | 306 |
| 448, L379R, A708K, P793_, Y857R, E386N, L404K | 307 |
| 449, L379R, A708K, P793_, D732N, E385P, Y857R | 308 |
| 450, L379R, A708K, P793_, D732N, E385P, Y857R, I658V | 309 |
| 451, L379R, A708K, P793_, D732N, E385P, Y857R, I658V, F399L | 310 |
| 452, L379R, A708K, P793_, D732N, E385P, Y857R, I658V, E386N | 311 |
| 453, L379R, A708K, P793_, D732N, E385P, Y857R, I658V, L404K | 312 |
| 454, L379R, A708K, P793_, T620P, E385P, Y857R, Q252K | 313 |
| 455, L379R, A708K, P793_, T620P, E385P, Y857R, I658V, Q252K | 314 |
| 456, L379R, A708K, P793_, T620P, E385P, Y857R, I658V, E386N, Q252K | 315 |
| 457, L379R, A708K, P793_, T620P, E385P, Y857R, I658V, F399L, Q252K | 316 |
| 458, L379R, A708K, P793_, T620P, E385P, Y857R, I658V, L404K, Q252K | 317 |
| 459, L379R, A708K, P793_, T620P, Y857R, I658V, E386N | 318 |
| 460, L379R, A708K, P793_, T620P, E385P, Q252K | 319 |
| 278 | 320 |
| 279 | 321 |
| 280 | 322 |
| 285 | 323 |
| 286 | 324 |
| 287 | 325 |
| 288 | 326 |
| 290 | 327 |
| 291 | 328 |
| 293 | 329 |
| 300 | 330 |
| 492 | 331 |
| 493 | 332 |
| 387 | 333 |
| 395 | 334 |
| 485 | 335 |
| 486 | 336 |
| 487 | 337 |
| 488 | 338 |
| 489 | 339 |
| 490 | 340 |
| 491 | 341 |
| 494 | 342 |
| 387 | 343 |
| 395 | 344 |
| 485 | 345 |
| 486 | 346 |
| 487 | 347 |
| 488 | 348 |
| 489 | 349 |
| 490 | 350 |
| 491 | 351 |
| 494 | 352 |
| 328, S867G | 4229 |
| 388, L379R + A708K + [P793] + X1 Helical2 swap | 4230 |
| 389, L379R + A708K + [P793] + X1 RuvC1 swap | 4231 |
| 390, L379R + A708K + [P793] + X1 RuvC2 swap | 4232 |

*Strain indicated numerically; changes, where indicated, are relative to SEQ ID NO: 2

In some embodiments, the CasX variant protein comprises between 400 and 2000 amino acids, between 500 and 1500 amino acids, between 700 and 1200 amino acids, between 800 and 1100 amino acids or between 900 and 1000 amino acids.

In other embodiments, the variant is RNA, and the one or more improved characteristics are independently selected from the group consisting of improved stability, improved solubility, improved resistance to nuclease activity, and improved binding to a binding partner.

In some embodiments, the variant is a guide RNA that binds to a CRISPR associated protein, and the one or more improved characteristics are independently selected from the group consisting of improved stability, improved solubility, improved resistance to nuclease activity, improved binding affinity to a Cas protein, improved binding affinity to a target DNA, improved gene editing, and improved specificity. In some embodiments, the variant is a guide RNA, wherein the variant has one or more altered activities compared to a reference. In some embodiments, the variant guide RNA has altered PAM specificity compared to a reference gRNA, for example has specificity for a different PAM sequence than the reference guide RNA.

In some embodiments, wherein the variant is a guide RNA variant, the one or more improved characteristics are improved compared to a reference gRNA of SEQ ID NO: 4. In other embodiments, wherein the variant is a guide RNA variant, the one or more improved characteristics are improved compared to a reference gRNA of SEQ ID NO: 5.

In still further embodiments, the variant is DNA. In some embodiments, the DNA variant encodes an RNA variant or protein variant. In certain embodiments, the encoded RNA or DNA has one or more improved characteristics as described herein.

In some embodiments, a biomolecule variant produced by the methods disclosed herein (e.g., protein variant, RNA variant, or DNA variant) has improved stability relative to a reference biomolecule. In some embodiments, improved stability of the variant results in expression of a higher steady state of the variant, or a larger fraction of expressed variant that remains folded in a functional conformation. In some embodiments, increased stability relative to the reference results in needing a lower concentration of the variant for use in a functional context, for example in gene editing. Thus, in some embodiments, the variant has improved efficiency compared to a reference in one or more functional contexts, which may include gene editing. In some embodiments, wherein the biomolecule is a Cas protein or guide RNA, the variant has improved stability of the variant Cas protein:guide-NA complex (e.g., a Cas protein:guide-RNA complex) relative to the reference biomolecule. Improved stability of the complex may, in some embodiments, lead to improved editing efficiency. In some embodiments, improved stability includes faster folding kinetics, or slower unfolding kinetics, or a larger free energy release upon folding, or a higher temperature at which 50% of the biomolecule is unfolded (Tm), or any combinations thereof, relative to the reference biomolecule. In some embodiments, folding kinetics of the biomolecule variant are improved relative to a reference biomolecule by at least about 1 kJ/mol, at least about 5 kJ/mol, at least about 10 kJ/mol, at least about 20 kJ/mol, at least about 30 kJ/mol, at least about 40 kJ/mol, at least about 50 kJ/mol, at least about 60 kJ/mol, at least about 70 kJ/mol, at least about 80 kJ/mol, at least about 90 kJ/mol, at least about 100 kJ/mol, at least about 150 kJ/mol, at least about 200 kJ/mol, at least about 250 kJ/mol, at least about 300 kJ/mol, at least about 350 kJ/mol, at least about 400 kJ/mol, at least about 450 kJ/mol, or at least about 500 kJ/mol. In some embodiments, improved stability of comprises a higher Tm relative to a reference biomolecule. In some embodiments, the Tm of the biomolecule protein variant is between about 20° C. to about 30° C., between about 30° C. to about 40° C., between about 40° C. to about 50° C., between about 50° C. to about 60° C., between about 60° C. to about 70° C., between about 70° C. to about 80° C., between about 80° C. to about 90° C. or between about 90° C. to about 100° C.

In some embodiments, a biomolecule variant has improved thermostability relative to a reference biomolecule. In some embodiments, a biomolecule variant as described herein has improved thermostability compared to a reference biomolecule at a temperature of at least 20° C., at least 22° C., at least 24° C., at least 26° C., at least 28° C., at least 30° C., at least 32° C., at least 34° C., at least 35° C., at least 36° C., at least 37° C., at least 38° C., at least 39° C., at least 40° C., at least 41° C., at least 42° C., at least 43° C., at least 44° C., at least 45° C., at least 46° C., at least 47° C., at least 48° C., at least 49° C., at least 50° C., at least 52° C., or greater, or between 10° C. to 60° C., between 10° C. to 50° C., between 10° C. to 40° C., between 20° C. to 40° C., or between 30° C. to 40° C. In certain variations, improved thermostability includes a higher proportion of the biomolecule remains soluble, a higher proportion of the biomolecule remains in a folded state, a higher proportion of the biomolecule retains activity, or a higher proportion of the biomolecule has a greater level of activity, or any combinations thereof, relative to the reference. In some embodiments, wherein the biomolecule is a Cas protein or guide RNA, a biomolecule variant has improved thermostability of a Cas protein:guide-NA complex compared to the reference biomolecule (e.g., a Cas protein:guide-RNA complex).

Methods of measuring characteristics of protein stability such as Tm and the free energy of unfolding are known to persons of ordinary skill in the art, and can be measured using standard biochemical techniques in vitro. For example, Tm may be measured using Differential Scanning calorimetry, a thermoanalytical technique in which the difference in the amount of heat required to increase the temperature of a sample and a reference is measured as a function of temperature. Alternatively, or in addition, biomolecule Tm may be measured using commercially available methods such as the ThermoFisher Protein Thermal Shift system. Alternatively, or in addition, circular dichroism may be used to measure the kinetics of folding and unfolding, as well as the Tm. Circular dichroism (CD) relies on the unequal absorption of left-handed and right-handed circularly polarized light by asymmetric molecules such as proteins. Certain structures of proteins, for example alpha-helices and beta-sheets, have characteristic CD spectra. Accordingly, in some embodiments, CD may be used to determine the secondary structure of a biomolecule.

Exemplary amino acid changes that can increase the stability of a protein variant relative to a reference protein may include, but are not limited to, amino acid changes that increase the number of hydrogen bonds within the protein variant, increase the number of disulfide bridges within the protein variant, increase the number of salt bridges within the protein variant, strengthen interactions between parts of the protein variant, increase the number of electrostatic interactions, or any combinations thereof, relative to the reference protein.

In some embodiments, the biomolecule variant has improved solubility compared to a reference biomolecule. In certain embodiments, wherein the biomolecule is a protein, an improvement in protein solubility leads to higher yield of protein from protein purification techniques such as purification from E. coli. Improved solubility of protein variants may, in some embodiments, enable more efficient activity in cells, as a more soluble protein may be less likely to aggregate in cells. Protein aggregates can in certain embodiments be toxic or burdensome on cells, and, without wishing to be bound by any theory, increased solubility of a protein variant may ameliorate this result of protein aggregation. Further, improved solubility of protein variants (such as CasX variants) may allow for the delivery of a higher effective dose of functional protein, for example in a desired gene editing application. In some embodiments, improved solubility of a protein variant relative to a reference protein results in improved yield of the protein variant during purification of a factor of at least about 5, at least about 10, at least about 20, at least about 30, at least about 40, at least about 50, at least about 60, at least about 70, at least about 80, at least about 90, at least about 100, at least about 250, at least about 500, or at least about 1000. In some embodiments, improved solubility of a protein variant relative to a reference protein improves activity of the protein variant in cells by a factor of at least about 1.1, at least about 1.2, at least about 1.3, at least about 1.4, at least about 1.5, at least about 1.6, at least about 1.7, at least about 1.8, at least about 1.9, at least about 2, at least about 2.1, at least about 2.2, at least about 2.3, at least about 2.4, at least about 2.5, at least about 2.6, at least about 2.7, at least about 2.8, at least about 2.9, at least about 3, at least about 3.5, at least about 4, at least about 4.5, at least about 5, at least about 5.5, at least about 6, at least about 6.5, at least about 7.0, at least about 7.5, at least about 8, at least about 8.5, at least about 9, at least about 9.5, at least about 10, at least about 11, at least about 12, at least about 13, at least about 14, or at least about 15. In some embodiments, the activity in cells of the variant relative to the CasX reference protein is improved by a factor of about 2, about 3, about 4, about 5, about 6, about 7, about 8, about 9, or about 10. In some embodiments, the protein variant is a CasX variant.

Methods of measuring protein solubility, and improvements thereof in protein variants, will be readily apparent to the person of ordinary skill in the art. For example, protein variant solubility can in some embodiments be measured by taking densitometry readings on a gel of the soluble fraction of lysed E. coli. Alternatively, or addition, improvements in protein variant solubility can be measured by measuring the maintenance of soluble protein product through the course of a full protein purification. For example, soluble protein product can be measured at one or more steps of gel affinity purification, tag cleavage, cation exchange purification, and/or running the protein on a sizing column. In some embodiments, the densitometry of every band of protein on a gel is read after each step in the purification process. Variant proteins with improved solubility may, in some embodiments, maintain a higher concentration at one or more steps in the protein purification process when compared to the reference protein, while an insoluble protein variant may be lost at one or more steps due to buffer exchanges, filtration steps, interactions with a purification column, and the like.

In some embodiments, improving the solubility of protein variants results in a higher yield in terms of mg/L of protein during protein purification when compared to a reference protein.

In some embodiments, improving the solubility of CasX variant proteins enables a greater amount of editing events compared to a less soluble protein when assessed in editing assays such as the EGFP disruption assays described herein.

In some embodiments, a biomolecule variant has improved resistance to degradative activity compared to a reference biomolecule, such as an improved resistance to nuclease (e.g., when the biomolecule is RNA) or protease (e.g., when the biomolecule is a protein) activity. In some such embodiments, increased resistance to degradative activity may result in improved functional activity.

In some embodiments, a biomolecule variant has improved affinity for a binding partner relative to a reference biomolecule. For example, in some embodiments, the biomolecule is a Cas protein, and the Cas protein variant has greater affinity for a gRNA than the reference Cas protein. In other embodiments, the biomolecule is a gRNA, and the gRNA variant has greater affinity for a Cas protein binding partner than the reference gRNA. In some embodiments, increased affinity of a biomolecule variant for a binding partner results in increased stability of the binding complex, such as when delivered to human cells. This increased stability can affect function and utility of the complex (e.g., in the cells of a subject, or intravenously). In some embodiments, increased affinity of a biomolecule variant and the resulting increased stability of the target complex results in lower levels of complex being needed to achieve the same functional outcome as when using the reference biomolecule. In certain embodiments, for example wherein the biomolecule is a gRNA or a Cas protein, the binding partner is DNA. In certain embodiments, a ribonucleoprotein complex comprising a gRNA variant or Cas protein variant has improved affinity for target nucleic acid (e.g., DNA or RNA), relative to the affinity of an RNP comprising a reference biomolecule. In some embodiments, the target nucleic acid is DNA, such as dsDNA or ssDNA. In other embodiments, the target nucleic acid is RNA. In some embodiments, the improved affinity of the RNP for the target nucleic acid comprises improved affinity for the target sequence, improved affinity for the PAM sequence, improved ability of the RNP to search the nucleic acid for the target sequence, or any combinations thereof. In some embodiments, the improved affinity for the target nucleic acid is the result of increased overall nucleic acid binding affinity. In some embodiments, wherein the biomolecule variant is a gRNA variant, one or more mutations in the gRNA variant may result in an increase of affinity of a Cas protein partner for the protospacer adjacent motif (PAM), thereby increasing affinity of the Cas protein partner for target nucleic acid, when complexed with the gRNA. In some embodiments, the protein variant has an altered PAM specificity (e.g., specificity for a different PAM) compared to a reference gRNA. Methods of evaluating biomolecule affinity for a binding partner are readily known to one of skill in the art, and may include, for example, fluorescence polarization, biolayer interferometry, electrophoretic mobility shift assays (EMSAs), filter binding, isothermal calorimetry (ITC), and surface plasmon resonance (SPR). In some embodiments, the K_{d} of a Cas protein variant for a gRNA (for example, a CasX variant protein for a gRNA) is increased relative to a reference Cas protein by a factor of at least about 1.1, at least about 1.2, at least about 1.3, at least about 1.4, at least about 1.5, at least about 1.6, at least about 1.7, at least about 1.8, at least about 1.9, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100.

In some embodiments, a Cas protein variant has improved specificity for a target nucleic acid (e.g., DNA such as dsDNA or ssDNA, or RNA) relative to a reference Cas protein. Improved specificity may include, for example, the degree to which a CRISPR/Cas system ribonucleoprotein complex cleaves off-target sequences that are similar, but not identical to the target nucleic acid. In some embodiments, a Cas protein variant has improved specificity for a target site within the target sequence that is complementary to the Spacer sequence of the gRNA. Methods of evaluating Cas protein (such as variant or reference) target specificity may include guide and Circularization for In vitro Reporting of Cleavage Effects by Sequencing (CIRCLE-seq); and assays used to detect and quantify indels (insertions and deletions) formed at selected off-target sites, such as mismatch-detection nuclease assays and next generation sequencing (NGS).

In some embodiments, wherein the biomolecule is a Cas protein, the Cas protein variant has improved ability of unwinding DNA relative to a reference Cas protein. In some embodiments, a Cas protein variant has enhanced DNA unwinding characteristics. Methods of measuring the ability of Cas proteins (such as variant or reference) to unwind DNA include, but are not limited to, in vitro assays that observe increased on rates of dsDNA targets in fluorescence polarization or biolayer interferometry. In some embodiments, affinity of a Cas protein variant (such as a CasX variant protein) for a target DNA molecule is increased relative to a reference Cas protein by a factor of at least about 1.1, at least about 1.2, at least about 1.3, at least about 1.4, at least about 1.5, at least about 1.6, at least about 1.7, at least about 1.8, at least about 1.9, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100.

In some embodiments, a ribonucleoprotein complex comprising a biomolecule variant as described herein has improved catalytic activity compared to a reference biomolecule. For example, wherein the biomolecule is a catalytic protein (such as a Cas protein), in certain embodiments the biomolecule variant has improved catalytic efficiency, specificity, or activity, compared to a reference biomolecule. Such catalytic activity may include cleavage of a nucleic acid sequence (e.g., DNA such as dsDNA or ssDNA, or RNA) wherein the biomolecule is a Cas protein. In some embodiments, improved affinity for nucleotides of a Cas protein variant also improves the function of catalytically inactive versions of the Cas protein variant (such as a CasX variant protein). In some embodiments, the catalytically inactive version of the Cas protein variant comprises one or mutations the DED motif in the RuvC. Catalytically dead Cas protein variants can, in some embodiments, be used for base editing or epigenetic modifications. With a higher affinity for nucleotides, in some embodiments catalytically dead Cas protein variants can find their target nucleic acid faster, remain bound to target nucleic acid for longer periods of time, bind target nucleic acid in a more stable fashion, or a combination thereof, thereby improving the function of the catalytically dead Cas protein variant.

In some embodiments, wherein a reduction of a certain characteristic is a desired trait, a biomolecule variant obtained through the methods described herein has said desired reduction. Such embodiments may result in a biomolecule variant that is better suited for a certain task.

In some embodiments, the one or more improved characteristics of the variant have an improvement by a factor of at least 1.1, at least 1.2, at least 1.3, at least 1.4, at least 1.5, at least 5, at least 10, at least 20, at least 30, at least 40, at least 50, at least 60, at least 70, at least 80, at least 90, at least 100, at least 125, at least 150, at least 175, or at least 200 fold compared to the reference biomolecule. In some embodiments, the improvement is between 1.1 to 5, between 1.1 to 10, between 1.1 to 20, between 5 to 10, between 5 to 20, between 5 to 50, between 10 to 20, between 10 to 30, between 10 to 50, between 10 to 100, between 50 to 100, between 50 to 150, between 50 to 200, between 70 to 100, between 70 to 150, between 100 to 150, between 100 to 200, or between 150 to 200 fold compared to the reference biomolecule. In still further embodiments, the one or more improved characteristics of the variant have an improvement of greater than 1.1, greater than 1.2, greater than 1.3, greater than 1.4, greater than 1.5, greater than 5, greater than 10, greater than 20, greater than 30, greater than 40, greater than 50, greater than 60, greater than 70, greater than 80, greater than 90, greater than 100, greater than 125, greater than 150, greater than 175, or greater than 200, compared to the reference biomolecule.

In some embodiments, the variant comprises at least one improved characteristic. In other embodiments, the variant comprises at least two improved characteristics. In further embodiments, the variant comprises at least three improved characteristics. In some embodiments, the variant comprises at least four improved characteristics. In still further embodiments, the variant comprises at least five, at least six, at least seven, at least eight, at least nine, at least ten, at least eleven, at least twelve, at least thirteen, or more improved characteristics.

In certain embodiments, wherein the variant is a protein, the variant comprises between 2 and 10,000 amino acids, between 100 and 10,000 amino acids, between 100 and 8,000 amino acids, between 100 and 6,000 amino acids, between 100 and 5,000 amino acids, between 100 and 4,000 amino acids, between 100 and 3,000 amino acids, between 100 and 2,000 amino acids, between 100 and 1,000 amino acids, between 100 and 1,500 amino acids, between 500 and 1,000 amino acids, between 500 and 1,500 amino acids, between 500 and 2,000 amino acids, between 1,000 and 3,000 amino acids, between 1,000 and 2,000 amino acids, between 2,000 and 10,000 amino acids, between 4,000 and 10,000 amino acids, between 6,000 and 10,000 amino acids, or between 8,000 and 10,000 amino acids.

In certain embodiments, wherein the variant is RNA or DNA, the variant comprises between 2 and 10,000 nucleotides, between 2 to 5,000 nucleotides, between 2 to 2,000 nucleotides, between 2 to 1,000 nucleotides, between 2 to 500 nucleotides, between 2 to 300 nucleotides, between 2 to 200 nucleotides, between 2 to 150 nucleotides, between 50 to 300 nucleotides, between 50 to 200 nucleotides, between 50 to 150 nucleotides, between 50 to 100 nucleotides, between 100 and 10,000 nucleotides, between 100 and 8,000 nucleotides, between 100 and 6,000 nucleotides, between 100 and 5,000 nucleotides, between 100 and 4,000 nucleotides, between 100 and 3,000 nucleotides, between 100 and 2,000 nucleotides, between 100 and 1,000 nucleotides, between 100 and 150 nucleotides, between 100 and 200 nucleotides, between 500 and 1,000 nucleotides, between 500 and 1,500 nucleotides, between 500 and 2,000 nucleotides, between 1,000 and 3,000 nucleotides, between 1,000 and 2,000 nucleotides, between 2,000 and 10,000 nucleotides, between 4,000 and 10,000 nucleotides, between 6,000 and 10,000 nucleotides, or between 8,000 and 10,000 nucleotides. In some embodiments, the variant is RNA. In certain embodiments, the RNA is a CRISPR associated guide RNA, the size of the variant excludes the size of the spacer region.

Table 2 provides the sequences of reference gRNAs tracr, cr and scaffold sequences. In some embodiments, the disclosure provides gNA sequences wherein the gNA has a scaffold comprising a sequence having at least one nucleotide modification relative to a reference gNA sequence having a sequence of any one of SEQ ID NOS: 4-16 of Table 2. It will be understood that in those embodiments wherein a vector comprises a DNA encoding sequence for a gNA, or where a gNA is a gDNA or a chimera of RNA and DNA, that thymine (T) bases can be substituted for the uracil (U) bases of any of the gNA sequence embodiments described herein.

TABLE 2
Reference gRNA tracr, cr and scaffold sequences

| SEQ ID NO. | Nucleotide Sequence |
|---|---|
| 4 | ACAUCUGGCGCGUUUAUUCCAUUACUUUGGAGCCAGUCCCAGCGACUAUGUCG |
| | UAUGGACGAAGCGCUUAUUUAUCGGAGAGAAACCGAUAAGUAAAACGCAUCAA |
| | AG |
| 5 | UACUGGCGCUUUUAUCUCAUUACUUUGAGAGCCAUCACCAGCGACUAUGUCGU |
| | AUGGGUAAAGCGCUUAUUUAUCGGAGAGAAAUCCGAUAAAUAAGAAGCAUCAA |
| | AG |
| 6 | ACAUCUGGCGCGUUUAUUCCAUUACUUUGGAGCCAGUCCCAGCGACUAUGUCG |
| | UAUGGACGAAGCGCUUAUUUAUCGGAGA |
| 7 | ACAUCUGGCGCGUUUAUUCCAUUACUUUGGAGCCAGUCCCAGCGACUAUGUCG |
| | UAUGGACGAAGCGCUUAUUUAUCGG |
| 8 | UACUGGCGCUUUUAUCUCAUUACUUUGAGAGCCAUCACCAGCGACUAUGUCGU |
| | AUGGGUAAAGCGCUUAUUUAUCGGAGA |
| 9 | UACUGGCGCUUUUAUCUCAUUACUUUGAGAGCCAUCACCAGCGACUAUGUCGU |
| | AUGGGUAAAGCGCUUAUUUAUCGG |
| 10 | GUUUACACACUCCCUCUCAUAGGGU |
| 11 | GUUUACACACUCCCUCUCAUGAGGU |
| 12 | UUUUACAUACCCCCUCUCAUGGGAU |
| 13 | GUUUACACACUCCCUCUCAUGGGGG |
| 14 | CCAGCGACUAUGUCGUAUGG |
| 15 | GCGCUUAUUUAUCGGAGAGAAAUCCGAUAAAUAAGAAGC |
| 16 | GGCGCUUUUAUCUCAUUACUUUGAGAGCCAUCACCAGCGACUAUGUCGUAUGG |
| | GUAAAGCGCUUAUUUAUCGGA |

In another aspect, the disclosure relates to guide nucleic acid variants (referred to herein alternatively as “gNA variant” or “gRNA variant”), which comprise one or more modifications relative to a reference gRNA scaffold. As used herein, “scaffold” refers to all parts to the gNA necessary for gNA function with the exception of the spacer sequence.

In some embodiments, a gNA variant comprises one or more nucleotide substitutions, insertions, deletions, or swapped or replaced regions relative to a reference gRNA sequence of the disclosure. In some embodiments, a mutation can occur in any region of a reference gRNA to produce a gNA variant. In some embodiments, the scaffold of the gNA variant sequence has at least 20%, at least 30%, at least 40%, at least 50%, at least 60%, or at least 70%, at least 80%, at least 85%, at least about 90%, at least about 95%, at least about 96%, at least about 97%, at least about 98%, or at least about 99% identity to the sequence of SEQ ID NO: 4 or SEQ ID NO: 5.

In some embodiments, a gNA variant comprises one or more nucleotide changes within one or more regions of the reference gRNA that improve a characteristic of the reference gRNA. Exemplary regions include the RNA triplex, the pseudoknot, the scaffold stem loop, and the extended stem loop. In some cases, the variant scaffold stem further comprises a bubble. In other cases, the variant scaffold further comprises a triplex loop region. In still other cases, the variant scaffold further comprises a 5′ unstructured region. In one embodiment, the gNA variant scaffold comprises a scaffold stem loop having at least 60% sequence identity to SEQ ID NO: 14. In another embodiment, the gNA variant comprises a scaffold stem loop having the sequence of CCAGCGACUAUGUCGUAGUGG (SEQ ID NO: 353).

All gNA variants that have one or more improved functions or characteristics, or add one or more new functions when the variant gNA is compared to a reference gRNA described herein, are envisaged as within the scope of the disclosure. A representative example of such a gNA variant created by the methods described herein is guide 174 (SEQ ID NO: 2238), the design of which is described in the Examples. In some embodiments, the gNA variant adds a new function to the RNP comprising the gNA variant. In some embodiments, the gNA variant has an improved characteristic selected from: improved stability; improved solubility; improved transcription of the gNA; improved resistance to nuclease activity; increased folding rate of the gNA; decreased side product formation during folding; increased productive folding; improved binding affinity to a CasX protein; improved binding affinity to a target DNA when complexed with a CasX protein; improved gene editing when complexed with a CasX protein; improved specificity of editing when complexed with a CasX protein; and improved ability to utilize a greater spectrum of one or more PAM sequences, including ATC, CTC, GTC, or TTC, in the editing of target DNA when complexed with a CasX protein, or any combination thereof. In some cases, the one or more of the improved characteristics of the gNA variant is at least about 1.1 to about 100,000-fold improved relative to the reference gNA of SEQ ID NO: 4 or SEQ ID NO: 5. In other cases, the one or more of the improved characteristics of the gNA variant is at least about 1.1, at least about 10, at least about 100, at least about 1000, at least about 10,000, at least about 100,000-fold or more improved relative to the reference gNA of SEQ ID NO: 4 or SEQ ID NO: 5. In other cases, the one or more of the improved characteristics of the gNA variant is about 1.1 to 100,00×, about 1.1 to 10,00×, about 1.1 to 1,000×, about 1.1 to 500×, about 1.1 to 100×, about 1.1 to 50×, about 1.1 to 20×, about 10 to 100,00×, about 10 to 10,00×, about 10 to 1,000×, about 10 to 500×, about 10 to 100×, about 10 to 50×, about 10 to 20×, about 2 to 70×, about 2 to 50×, about 2 to 30×, about 2 to 20×, about 2 to 10×, about 5 to 50×, about 5 to 30×, about 5 to 10×, about 100 to 100,00×, about 100 to 10,00×, about 100 to 1,000×, about 100 to 500×, about 500 to 100,00×, about 500 to 10,00×, about 500 to 1,000×, about 500 to 750×, about 1,000 to 100,00×, about 10,000 to 100,00×, about 20 to 500×, about 20 to 250×, about 20 to 200×, about 20 to 100×, about 20 to 50×, about 50 to 10,000×, about 50 to 1,000×, about 50 to 500×, about 50 to 200×, or about 50 to 100×, improved relative to the reference gNA of SEQ ID NO: 4 or SEQ ID NO: 5. In other cases, the one or more of the improved characteristics of the gNA variant is about 1.1×, 1.2×, 1.3×, 1.4×, 1.5×, 1.6×, 1.7×, 1.8×, 1.9×, 2×, 3×, 4×, 5×, 6×, 7×, 8×, 9×, 10×, 11×, 12×, 13×, 14×, 15×, 16×, 17×, 18×, 19×, 20×, 25×, 30×, 40×, 45×, 50×, 55×, 60×, 70×, 80×, 90×, 100×, 110×, 120×, 130×, 140×, 150×, 160×, 170×, 180×, 190×, 200×, 210×, 220×, 230×, 240×, 250×, 260×, 270×, 280×, 290×, 300×, 310×, 320×, 330×, 340×, 350×, 360×, 370×, 380×, 390×, 400×, 425×, 450×, 475×, or 500× improved relative to the reference gNA of SEQ ID NO: 4 or SEQ ID NO: 5.

In some embodiments, a gNA variant can be created by subjecting a reference gRNA to a one or more mutagenesis methods, such as the mutagenesis methods described herein, below, which may include Deep Mutational Evolution (DME), deep mutational scanning (DMS), error prone PCR, cassette mutagenesis, random mutagenesis, staggered extension PCR, gene shuffling, or domain swapping, in order to generate the gNA variants of the disclosure. The activity of reference gRNAs may be used as a benchmark against which the activity of gNA variants are compared, thereby measuring improvements in function of gNA variants. In other embodiments, a reference gRNA may be subjected to one or more deliberate, targeted mutations, substitutions, or domain swaps in order to produce a gNA variant, for example a rationally designed variant. Exemplary gRNA variants produced by such methods are described in the Examples and representative sequences of gNA scaffolds are presented in Table 3.

In some embodiments, the gNA variant comprises one or more modifications compared to a reference guide nucleic acid scaffold sequence, wherein the one or more modification is selected from: at least one nucleotide substitution in a region of the gNA variant; at least one nucleotide deletion in a region of the gNA variant; at least one nucleotide insertion in a region of the gNA variant; a substitution of all or a portion of a region of the gNA variant; a deletion of all or a portion of a region of the gNA variant; or any combination of the foregoing. In some cases, the modification is a substitution of 1 to 15 consecutive or non-consecutive nucleotides in the gNA variant in one or more regions. In other cases, the modification is a deletion of 1 to 10 consecutive or non-consecutive nucleotides in the gNA variant in one or more regions. In other cases, the modification is an insertion of 1 to 10 consecutive or non-consecutive nucleotides in the gNA variant in one or more regions. In other cases, the modification is a substitution of the scaffold stem loop or the extended stem loop with an RNA stem loop sequence from a heterologous RNA source with proximal 5′ and 3′ ends. In some embodiments, the gNA variant comprises an extended stem loop region comprising at least 10, at least 100, at least 500, at least 1000, or at least 10,000 nucleotides. In some embodiments, the heterologous stem loop increases the stability of the gNA. In some embodiments, the heterologous RNA stem loop is capable of binding a protein, an RNA structure, a DNA sequence, or a small molecule. In some embodiments, an exogenous stem loop region comprises an RNA stem loop or hairpin, for example a thermostable RNA such as MS2 (ACAUGAGGAUUACCCAUGU; SEQ ID NO: 354), Qβ (UGCAUGUCUAAGACAGCA; SEQ ID NO: 355), U1 hairpin II (AAUCCAUUGCACUCCGGAUU; SEQ ID NO: 356), Uvsx (CCUCUUCGGAGG; SEQ ID NO: 357), PP7 (AGGAGUUUCUAUGGAAACCCU; SEQ ID NO: 358), Phage replication loop (AGGUGGGACGACCUCUCGGUCGUCCUAUCU; SEQ ID NO: 359), Kissing loop_a (UGCUCGCUCCGUUCGAGCA; SEQ ID NO: 360), Kissing loop_b1 (UGCUCGACGCGUCCUCGAGCA; SEQ ID NO: 361), Kissing loop_b2 (UGCUCGUUUGCGGCUACGAGCA; SEQ ID NO: 362), G quadriplex M3q (AGGGAGGGAGGGAGAGG; SEQ ID NO: 363), G quadriplex telomere basket (GGUUAGGGUUAGGGUUAGG; SEQ ID NO: 364), Sarcin-ricin loop (CUGCUCAGUACGAGAGGAACCGCAG; SEQ ID NO: 365) or Pseudoknots (UACACUGGGAUCGCUGAAUUAGAGAUCGGCGUCCUUUCAUUCUAUAUACUUUGG AGUUUUAAAAUGUCUCUAAGUACA; SEQ ID NO: 366). In some embodiments, an exogenous stem loop comprises a long non-coding RNA (lncRNA). As used herein, a lncRNA refers to a non-coding RNA that is longer than approximately 200 bp in length. In some embodiments, the 5′ and 3′ ends of the exogenous stem loop are base paired, i.e., interact to form a region of duplex RNA. In some embodiments, the 5′ and 3′ ends of the exogenous stem loop are base paired, and one or more regions between the 5′ and 3′ ends of the exogenous stem loop are not base paired.

In some cases, a gNA variant of the disclosure comprises two or more modifications in one region. In other cases, a gNA variant of the disclosure comprises modifications in two or more regions. In other cases, a gNA variant comprises any combination of the foregoing modifications described in this paragraph. In some embodiments, exemplary modifications of gNA of the disclosure include the modifications of Table 3.

In some embodiments, a 5′ G is added to a gNA variant sequence for expression in vivo, as transcription from a U6 promoter is more efficient and more consistent with regard to the start site when the +1 nucleotide is a G. In other embodiments, two 5′ Gs are added to a gNA variant sequence for in vitro transcription to increase production efficiency, as T7 polymerase strongly prefers a G in the +1 position and a purine in the +2 position. In some cases, the 5′ G bases are added to the reference scaffolds of Table 2. In other cases, the 5′ G bases are added to the variant scaffolds of Table 3.

Table 3 provides exemplary gNA variant scaffold sequences of the disclosure created by the methods of the disclosure. In Table 3, (−) indicates a deletion at the specified position(s) relative to the reference sequence of SEQ ID NO: 5, (+) indicates an insertion of the specified base(s) at the position indicated relative to SEQ ID NO: 5, (:) indicates the range of bases at the specified start:stop coordinates of a deletion or substitution relative to SEQ ID NO: 5, and multiple insertions, deletions or substitutions are separated by commas; e.g., A14C, T17G. In some embodiments, the gNA variant scaffold comprises any one of the sequences listed in Table 3, or SEQ ID NOS: 2101-2280, or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto. In some embodiments, the gNA variant comprises one or more additional changes to a sequence of any one of SEQ ID NOs: 2201-2280. In some embodiments, the gNA variant comprises the sequence of any one of SEQ ID NOS: 2236, 2237, 2238, 2241, 2244, 2248, 2249, or 2259-2280, or having at least about 80%, at least about 90%, at least about 95%, at least about 96%, at least about 97%, at least about 98%, at least about 99% identity thereto. In some embodiments, the gNA variant comprises one or more additional changes to a sequence of any one of SEQ ID NOs: 2201-2280. In some embodiments of the gNA variants of the disclosure, the gNA variant comprises at least one modification, wherein the at least one modification compared to the reference guide scaffold of SEQ ID NO: 5 is selected from one or more of: (a) a C18G substitution in the triplex loop; (b) a G55 insertion in the stem bubble; (c) a U1 deletion; (d) a modification of the extended stem loop wherein (i) a 6 nt loop and 13 loop-proximal base pairs are replaced by a Uvsx hairpin; and (ii) a deletion of A99 and a substitution of G65U that results in a loop-distal base that is fully base-paired. In some embodiments, the gNA variant comprises the sequence of any one of SEQ ID NOS: 2236, 2237, 2238, 2241, 2244, 2248, 2249, or 2259-2280. It will be understood that in those embodiments wherein a vector comprises a DNA encoding sequence for a gNA, or where a gNA is a gDNA or a chimera of RNA and DNA, that thymine (T) bases can be substituted for the uracil (U) bases of any of the gNA sequence embodiments described herein.

TABLE 3
Exemplary gNA Variant Scaffold Sequences

| SEQ | | |
|---|---|---|
| ID | NAME or | |
| NO: | Modification | NUCLEOTIDE SEQUENCE |
| 2101 | phage | UACUGGCGCUUUUAUCUCAUUACUUUGAGAGCCAUCACCAGCGACUA |
| | replication | UGUCGUAUGGGUAAAGCGCAGGUGGGACGACCUCUCGGUCGUCCUAU |
| | stable | CUGAAGCAUCAAAG |
| 2102 | Kissing | UACUGGCGCUUUUAUCUCAUUACUUUGAGAGCCAUCACCAGCGACUA |
| | loop_b1 | UGUCGUAUGGGUAAAGCGCUGCUCGACGCGUCCUCGAGCAGAAGCAU |
| | | CAAAG |
| 2103 | Kissing | UACUGGCGCUUUUAUCUCAUUACUUUGAGAGCCAUCACCAGCGACUA |
| | loop_a | UGUCGUAUGGGUAAAGCGCUGCUCGCUCCGUUCGAGCAGAAGCAUCA |
| | | AAG |
| 2104 | 32, uvsX | GUACUGGCGCUUUUAUCUCAUUACUUUGAGAGCCAUCACCAGCGACU |
| | hairpin | AUGUCGUAUGGGUAAAGCGCCCUCUUCGGAGGGAAGCAUCAAAG |
| 2105 | PP7 | UACUGGCGCUUUUAUCUCAUUACUUUGAGAGCCAUCACCAGCGACUA |
| | | UGUCGUAUGGGUAAAGCGCAGGAGUUUCUAUGGAAACCCUGAAGCAU |
| | | CAAAG |
| 2106 | 64, trip mut, | GUACUGGCGCCUUUAUCUCAUUACUUUGAGAGCCAUCACCAGCGACU |
| | extended stem | AUGUCGUAUGGGUAAAGCGCUUACGGACUUCGGUCCGUAAGAAGCAU |
| | truncation | CAAAG |
| 2107 | hyperstable | UACUGGCGCUUUUAUCUCAUUACUUUGAGAGCCAUCACCAGCGACUA |
| | tetraloop | UGUCGUAUGGGUAAAGCGCUGCGCUUGCGCAGAAGCAUCAAAG |
| 2108 | C18G | UACUGGCGCUUUUAUCUGAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAG |
| 2109 | T17G | UACUGGCGCUUUUAUCGCAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAG |
| 2110 | CUUCGG | UACUGGCGCUUUUAUCUCAUUACUUUGAGAGCCAUCACCAGCGACUA |
| | loop | UGUCGUAUGGGUAAAGCGCUUAUUUAUCGGAGACUUCGGUCCGAUAA |
| | | AUAAGAAGCAUCAAAG |
| 2111 | MS2 | UACUGGCGCUUUUAUCUCAUUACUUUGAGAGCCAUCACCAGCGACUA |
| | | UGUCGUAUGGGUAAAGCGCACAUGAGGAUUACCCAUGUGAAGCAUCA |
| | | AAG |
| 2112 | -1, A2G, -78, | GCUGGCGCUUUUAUCUCAUUACUUUGAGAGCCAUCACCAGCGACUAU |
| | G77T | GUCGUAUGGGUAAAGCGCUUAUUUAUCGUGAGAAAUCCGAUAAAUAA |
| | | GAAGCAUCAAAG |
| 2113 | QB | UACUGGCGCUUUUAUCUCAUUACUUUGAGAGCCAUCACCAGCGACUA |
| | | UGUCGUAUGGGUAAAGCGCUGCAUGUCUAAGACAGCAGAAGCAUCAA |
| | | AG |
| 2114 | 45, 44 hairpin | UACUGGCGCUUUUAUCUCAUUACUUUGAGAGCCAUCACCAGCGACUA |
| | | UGUCGUAUGGGUAAAGCGCAGGGCUUCGGCCGAAGCAUCAAAG |
| 2115 | U1A | UACUGGCGCUUUUAUCUCAUUACUUUGAGAGCCAUCACCAGCGACUA |
| | | UGUCGUAUGGGUAAAGCGCAAUCCAUUGCACUCCGGAUUGAAGCAUC |
| | | AAAG |
| 2116 | A14C, T17G | UACUGGCGCUUUUCUCGCAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAG |
| 2117 | CUUCGG | UACUGGCGCUUUUAUCUCAUUACUUUGAGAGCCAUCACCAGCGACUA |
| | loop modified | UGUCGUAUGGGUAAAGCGCUUAUUUAUCGGACUUCGGUCCGAUAAAU |
| | | AAGAAGCAUCAAAG |
| 2118 | Kissing | UACUGGCGCUUUUAUCUCAUUACUUUGAGAGCCAUCACCAGCGACUA |
| | loop_b2 | UGUCGUAUGGGUAAAGCGCUGCUCGUUUGCGGCUACGAGCAGAAGCA |
| | | UCAAAG |
| 2119 | -76:78, -83:87 | UACUGGCGCUUUUAUCUCAUUACUUUGAGAGCCAUCACCAGCGACUA |
| | | UGUCGUAUGGGUAAAGCGCUUAUUUAUCGAGAGAUAAAUAAGAAGCA |
| | | UCAAAG |
| 2120 | -4 | UACGGCGCUUUUAUCUCAUUACUUUGAGAGCCAUCACCAGCGACUAU |
| | | GUCGUAUGGGUAAAGCGCUUAUUUAUCGGAGAGAAAUCCGAUAAAUA |
| | | AGAAGCAUCAAAG |
| 2121 | extended stem | UACUGGCGCCUUUUAUCUCAUUACUUUGAGAGCCAUCACCAGCGACU |
| | truncation | AUGUCGUAUGGGUAAAGCGCUUACGGACUUCGGUCCGUAAGAAGCAU |
| | | CAAAG |
| 2122 | C55 | UACUGGCGCUUUUAUCUCAUUACUUUGAGAGCCAUCACCAGCGACUA |
| | | UGUCGUAUCGGUAAAGCGCUUAUUUAUCGGAGAGAAAUCCGAUAAAU |
| | | AAGAAGCAUCAAAG |
| 2123 | trip mut | UACUGGCGCCUUUAUCUCAUUACUUUGAGAGCCAUCACCAGCGACUA |
| | | UGUCGUAUGGGUAAAGCGCUUAUUUAUCGGACUUCGGUCCGAUAAAU |
| | | AAGAAGCAUCAAAG |
| 2124 | -76:78 | UACUGGCGCUUUUAUCUCAUUACUUUGAGAGCCAUCACCAGCGACUA |
| | | UGUCGUAUGGGUAAAGCGCUUAUUUAUCGAGAAAUCCGAUAAAUAAG |
| | | AAGCAUCAAAG |
| 2125 | -1:5 | GCGCUUUUAUCUCAUUACUUUGAGAGCCAUCACCAGCGACUAUGUCG |
| | | UAUGGGUAAAGCGCUUAUUUAUCGGAGAGAAAUCCGAUAAAUAAGAA |
| | | GCAUCAAAG |
| 2126 | -83:87 | UACUGGCGCUUUUAUCUCAUUACUUUGAGAGCCAUCACCAGCGACUA |
| | | UGUCGUAUGGGUAAAGCGCUUAUUUAUCGGAGAGAGAUAAAUAAGAA |
| | | GCAUCAAAG |
| 2127 | =+G28, A82T, | UACUGGCGCUUUUAUCUCAUUACUUUGGAGAGCCAUCACCAGCGACU |
| | -84, | AUGUCGUAUGGGUAAAGCGCUUAUUUAUCGGAGAGUAUCCGAUAAAU |
| | | AAGAAGCAUCAAAG |
| 2128 | =+51T | UACUGGCGCUUUUAUCUCAUUACUUUGAGAGCCAUCACCAGCGACUA |
| | | UGUUCGUAUGGGUAAAGCGCUUAUUUAUCGGAGAGAAAUCCGAUAAA |
| | | UAAGAAGCAUCAAAG |
| 2129 | -1:4, +G5A, | AGCGCUUUUAUCUCAUUACUUUGAGAGCCAUCACCAGCGACUAUGUC |
| | +G86, | GUAUGGGUAAAGCGCUUAUUUAUCGGAGAGAAAUGCCGAUAAAUAAG |
| | | AAGCAUCAAAG |
| 2130 | =+A94 | UACUGGCGCUUUUAUCUCAUUACUUUGAGAGCCAUCACCAGCGACUA |
| | | UGUCGUAUGGGUAAAGCGCUUAUUUAUCGGAGAGAAAUCCGAUAAAA |
| | | UAAGAAGCAUCAAAG |
| 2131 | =+G72 | UACUGGCGCUUUUAUCUCAUUACUUUGAGAGCCAUCACCAGCGACUA |
| | | UGUCGUAUGGGUAAAGCGCUUAUUGUAUCGGAGAGAAAUCCGAUAAA |
| | | UAAGAAGCAUCAAAG |
| 2132 | shorten front, | GCGCUUUUAUCUCAUUACUUUGAGAGCCAUCACCAGCGACUAUGUCG |
| | CUUCGG | UAUGGGUAAAGCGCUUAUUUAUCGGACUUCGGUCCGAUAAAUAAGCG |
| | loop modified. | CAUCAAAG |
| | extend | |
| | extended | |
| 2133 | A14C | UACUGGCGCUUUUCUCUCAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAG |
| 2134 | -1:3, +G3 | GUGGCGCUUUUAUCUCAUUACUUUGAGAGCCAUCACCAGCGACUAUG |
| | | UCGUAUGGGUAAAGCGCUUAUUUAUCGGAGAGAAAUCCGAUAAAUAA |
| | | GAAGCAUCAAAG |
| 2135 | =+C45, +T46 | UACUGGCGCUUUUAUCUCAUUACUUUGAGAGCCAUCACCAGCGACCU |
| | | UAUGUCGUAUGGGUAAAGCGCUUAUUUAUCGGAGAGAAAUCCGAUAA |
| | | AUAAGAAGCAUCAAAG |
| 2136 | CUUCGG | GAUGGCGCUUUUAUCUCAUUACUUUGAGAGCCAUCACCAGCGACUAU |
| | loop modified, | GUCGUAUGGGUAAAGCGCUUAUUUAUCGGACUUCGGUCCGAUAAAUA |
| | fun start | AGAAGCAUCAAAG |
| 2137 | -93:94 | UACUGGCGCUUUUAUCUCAUUACUUUGAGAGCCAUCACCAGCGACUA |
| | | UGUCGUAUGGGUAAAGCGCUUAUUUAUCGGAGAGAAAUCCGAUAAAA |
| | | GAAGCAUCAAAG |
| 2138 | =+T45 | UACUGGCGCUUUUAUCUCAUUACUUUGAGAGCCAUCACCAGCGAUCU |
| | | AUGUCGUAUGGGUAAAGCGCUUAUUUAUCGGAGAGAAAUCCGAUAAA |
| | | UAAGAAGCAUCAAAG |
| 2139 | -69, -94 | UACUGGCGCUUUUAUCUCAUUACUUUGAGAGCCAUCACCAGCGACUA |
| | | UGUCGUAUGGGUAAAGGCUUAUUUAUCGGAGAGAAAUCCGAUAAAAA |
| | | GAAGCAUCAAAG |
| 2140 | -94 | UACUGGCGCUUUUAUCUCAUUACUUUGAGAGCCAUCACCAGCGACUA |
| | | UGUCGUAUGGGUAAAGCGCUUAUUUAUCGGAGAGAAAUCCGAUAAAA |
| | | AGAAGCAUCAAAG |
| 2141 | modified | UACUGGCGCUUUAUCUCAUUACUUUGAGAGCCAUCACCAGCGACUAU |
| | CUUCGG, | GUCGUAUGGGUAAAGCGCUUAUUUAUCGGACUUCGGUCCGAUAAAUA |
| | minus T in 1st | AGAAGCAUCAAAG |
| | triplex | |
| 2142 | -1:4, +C4, | CGGCGCUUUUCUCGCAUUACUUUGAGAGCCAUCACCAGCGACUAUGU |
| | A14C, T17G, | CGUAUGGGUAAAGCGCUUAUUGUAUCGAGAGAUAAAUAAGAAGCAUC |
| | +G72, -76:78, | AAAG |
| | -83:87 | |
| 2143 | T1C, -73 | CACUGGCGCUUUUAUCUCAUUACUUUGAGAGCCAUCACCAGCGACUA |
| | | UGUCGUAUGGGUAAAGCGCUUAUUUUCGGAGAGAAAUCCGAUAAAUA |
| | | AGAAGCAUCAAAG |
| 2144 | Scaffold | UACUGGCGCUUUAUCUCAUUACUUUGAGAGCCAUCACCAGCGACUUC |
| | uuCG, stem | GGUCGUAUGGGUAAAGCGCUUAUGUAUCGGCUUCGGCCGAUACAUAA |
| | uuCG. Stem | GAAGCAUCAAAG |
| | swap, t | |
| | shorten | |
| 2145 | Scaffold | UACUGGCGCUUUUAUCUCAUUACUUUGAGAGCCAUCACCAGCGACUU |
| | uuCG, stem | CGGUCGUAUGGGUAAAGCGCUUAUGUAUCGGCUUCGGCCGAUACAUA |
| | uuCG. Stem | AGAAGCAUCAAAG |
| | swap | |
| 2146 | =+G60 | UACUGGCGCUUUUAUCUCAUUACUUUGAGAGCCAUCACCAGCGACUA |
| | | UGUCGUAUGGGUGAAAGCGCUUAUUUAUCGGAGAGAAAUCCGAUAAA |
| | | UAAGAAGCAUCAAAG |
| 2147 | no stem | UACUGGCGCUUUUAUCUCAUUACUUUGAGAGCCAUCACCAGCGACUU |
| | Scaffold | CGGUCGUAUGGGUAAAG |
| | uuCG | |
| 2148 | no stem | GAUGGGCUUUUAUCUCAUUACUUUGAGAGCCAUCACCAGCGACUUCG |
| | Scaffold | GUCGUAUGGGUAAAG |
| | uuCG, fun | |
| | start | |
| 2149 | Scaffold | GAUGGGCUUUUAUCUCAUUACUUUGAGAGCCAUCACCAGCGACUUCG |
| | uuCG, stem | GUCGUAUGGGUAAAGCGCUUAUUUAUCGGCUUCGGCCGAUAAAUAAG |
| | uuCG, fun | AAGCAUCAAAG |
| | start | |
| 2150 | Pseudoknots | UACUGGCGCUUUUAUCUCAUUACUUUGAGAGCCAUCACCAGCGACUA |
| | | UGUCGUAUGGGUAAAGCGCUACACUGGGAUCGCUGAAUUAGAGAUCG |
| | | GCGUCCUUUCAUUCUAUAUACUUUGGAGUUUUAAAAUGUCUCUAAGU |
| | | ACAGAAGCAUCAAAG |
| 2151 | Scaffold | GGCGCUUUUAUCUCAUUACUUUGAGAGCCAUCACCAGCGACUUCGGU |
| | uuCG, stem | CGUAUGGGUAAAGCGCUUAUUUAUCGGCUUCGGCCGAUAAAUAAGAA |
| | uuCG | GCAUCAAAG |
| 2152 | Scaffold | GCUGGCGCUUUUAUCUCAUUACUUUGAGAGCCAUCACCAGCGACUUC |
| | uuCG, stem | GGUCGUAUGGGUAAAGCGCUUAUUUAUCGGCUUCGGCCGAUAAAUAA |
| | uuCG, no start | GAAGCAUCAAAG |
| 2153 | Scaffold | UACUGGCGCUUUUAUCUCAUUACUUUGAGAGCCAUCACCAGCGACUU |
| | uuCG | CGGUCGUAUGGGUAAAGCGCUUAUUUAUCGGAGAGAAAUCCGAUAAA |
| | | UAAGAAGCAUCAAAG |
| 2154 | =+GCTC36 | UACUGGCGCUUUUAUCUCAUUACUUUGAGAGCCAUGCUCCACCAGCG |
| | | ACUAUGUCGUAUGGGUAAAGCGCUUAUUUAUCGGAGAGAAAUCCGAU |
| | | AAAUAAGAAGCAUCAAAG |
| 2155 | G quadriplex | UACUGGCGCUUUUAUCUCAUUACUUUGAGAGCCAUCACCAGCGACUA |
| | telomere | UGUCGUAUGGGUAAAGCGGGGUUAGGGUUAGGGUUAGGGAAGCAUCA |
| | basket+ ends | AAG |
| 2156 | G quadriplex | UACUGGCGCUUUUAUCUCAUUACUUUGAGAGCCAUCACCAGCGACUA |
| | M3q | UGUCGUAUGGGUAAAGCGGAGGGAGGGAGGGAGAGGGAAAGCAUCAA |
| | | AG |
| 2157 | G quadriplex | UACUGGCGCUUUUAUCUCAUUACUUUGAGAGCCAUCACCAGCGACUA |
| | telomere | UGUCGUAUGGGUAAAGCGUUGGGUUAGGGUUAGGGUUAGGGAAAAGC |
| | basket no ends | AUCAAAG |
| 2158 | 45, 44 hairpin | UACUGGCGCUUUUAUCUCAUUACUUUGAGAGCCAUCACCAGCGACUA |
| | (old version) | UGUCGUAUGGGUAAAGCGC--------AGGGCUUCGGCCG------- |
| | | --GAAGCAUCAAAG |
| 2159 | Sarcin-ricin | UACUGGCGCUUUUAUCUCAUUACUUUGAGAGCCAUCACCAGCGACUA |
| | loop | UGUCGUAUGGGUAAAGCGCCUGCUCAGUACGAGAGGAACCGCAGGAA |
| | | GCAUCAAAG |
| 2160 | uvsX, C18G | UACUGGCGCUUUUAUCUGAUUACUUUGAGAGCCAUCACCAGCGACUA |
| | | UGUCGUAUGGGUAAAGCGCCCUCUUCGGAGGGAAGCAUCAAAG |
| 2161 | truncated stem | UACUGGCGCCUUUAUCUGAUUACUUUGAGAGCCAUCACCAGCGACUA |
| | loop, C18G, | UGUCGUAUGGGUAAAGCGCUUACGGACUUCGGUCCGUAAGAAGCAUC |
| | trip mut | AAAG |
| | (T10C) | |
| 2162 | short phage | UACUGGCGCUUUUAUCUGAUUACUUUGAGAGCCAUCACCAGCGACUA |
| | rep, C18G | UGUCGUAUGGGUAAAGCGCGGACGACCUCUCGGUCGUCCGAAGCAUC |
| | | AAAG |
| 2163 | phage rep | UACUGGCGCUUUUAUCUGAUUACUUUGAGAGCCAUCACCAGCGACUA |
| | loop, C18G | UGUCGUAUGGGUAAAGCGCAGGUGGGACGACCUCUCGGUCGUCCUAU |
| | | CUGAAGCAUCAAAG |
| 2164 | =+G18, | UACUGGCGCCUUUAUCUGCAUUACUUUGAGAGCCAUCACCAGCGACU |
| | stacked onto | AUGUCGUAUGGGUAAAGCGCUUACGGACUUCGGUCCGUAAGAAGCAU |
| | 64 | CAAAG |
| 2165 | truncated stem | GCUGGCGCUUUUAUCUGAUUACUUUGAGAGCCAUCACCAGCGACUAU |
| | loop, C18G, -1 | GUCGUAUGGGUAAAGCGCUUACGGACUUCGGUCCGUAAGAAGCAUCA |
| | A2G | AAG |
| 2166 | phage rep | UACUGGCGCCUUUAUCUGAUUACUUUGAGAGCCAUCACCAGCGACUA |
| | lpop, C18G, | UGUCGUAUGGGUAAAGCGCAGGUGGGACGACCUCUCGGUCGUCCUAU |
| | trip mut | CUGAAGCAUCAAAG |
| | (T10C) | |
| 2167 | short phage | UACUGGCGCCUUUAUCUGAUUACUUUGAGAGCCAUCACCAGCGACUA |
| | rep, C18G, | UGUCGUAUGGGUAAAGCGCGGACGACCUCUCGGUCGUCCGAAGCAUC |
| | trip mut | AAAG |
| | (T10C) | |
| 2168 | uvsX, trip mut | UACUGGCGCCUUUAUCUCAUUACUUUGAGAGCCAUCACCAGCGACUA |
| | (T10C) | UGUCGUAUGGGUAAAGCGCCCUCUUCGGAGGGAAGCAUCAAAG |
| 2169 | truncated stem | UACUGGCGCUUUUAUCUCAUUACUUUGAGAGCCAUCACCAGCGACUA |
| | loop | UGUCGUAUGGGUAAAGCGCUUACGGACUUCGGUCCGUAAGAAGCAUC |
| | | AAAG |
| 2170 | =+A17, | UACUGGCGCCUUUAUCAUCAUUACUUUGAGAGCCAUCACCAGCGACU |
| | stacked onto | AUGUCGUAUGGGUAAAGCGCUUACGGACUUCGGUCCGUAAGAAGCAU |
| | 64 | CAAAG |
| 2171 | 3′ HDV | UACUGGCGCUUUUAUCUCAUUACUUUGAGAGCCAUCACCAGCGACUA |
| | genomic | UGUCGUAUGGGUAAAGCGCUUAUUUAUCGGAGAGAAAUCCGAUAAAU |
| | ribozyme | AAGAAGCAUCAAAGGGCCGGCAUGGUCCCAGCCUCCUCGCUGGCGCC |
| | | GGCUGGGCAACAUUCCGAGGGGACCGUCCCCUCGGUAAUGGCGAAUG |
| | | GGACCC |
| 2172 | phage rep | UACUGGCGCCUUUAUCUCAUUACUUUGAGAGCCAUCACCAGCGACUA |
| | loop, trip mut | UGUCGUAUGGGUAAAGCGCAGGUGGGACGACCUCUCGGUCGUCCUAU |
| | (T10C) | CUGAAGCAUCAAAG |
| 2173 | -79:80 | UACUGGCGCUUUUAUCUCAUUACUUUGAGAGCCAUCACCAGCGACUA |
| | | UGUCGUAUGGGUAAAGCGCUUAUUUAUCGGAGAAAUCCGAUAAAUAA |
| | | GAAGCAUCAAAG |
| 2174 | short phage | UACUGGCGCCUUUAUCUCAUUACUUUGAGAGCCAUCACCAGCGACUA |
| | rep, trip mut | UGUCGUAUGGGUAAAGCGCGGACGACCUCUCGGUCGUCCGAAGCAUC |
| | (T10C) | AAAG |
| 2175 | extra | UACUGGCGCUUUUAUCUCAUUACUUUGAGAGCCAUCACCAGCGACUA |
| | truncated stem | UGUCGUAUGGGUAAAGCGCCGGACUUCGGUCCGGAAGCAUCAAAG |
| | loop | |
| 2176 | T17G, C18G | UACUGGCGCUUUUAUCGGAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAG |
| 2177 | short phage | UACUGGCGCUUUUAUCUCAUUACUUUGAGAGCCAUCACCAGCGACUA |
| | rep | UGUCGUAUGGGUAAAGCGCGGACGACCUCUCGGUCGUCCGAAGCAUC |
| | | AAAG |
| 2178 | uvsX, C18G, -1 | GCUGGCGCUUUUAUCUGAUUACUUUGAGAGCCAUCACCAGCGACUAU |
| | A2G | GUCGUAUGGGUAAAGCGCCCUCUUCGGAGGGAAGCAUCAAAG |
| 2179 | uvsX, C18G, | GCUGGCGCCUUUAUCUGAUUACUUUGAGAGCCAUCACCAGCGACUAU |
| | trip mut | GUCGUAUGGGUAAAGCUCCCUCUUCGGAGGGAGCAUCAAAG |
| | (T10C), -1 | |
| | A2G, HDV | |
| | -99 G65U | |
| 2180 | 3′ HDV | UACUGGCGCUUUUAUCUCAUUACUUUGAGAGCCAUCACCAGCGACUA |
| | antigenomic | UGUCGUAUGGGUAAAGCGCUUAUUUAUCGGAGAGAAAUCCGAUAAAU |
| | ribozyme | AAGAAGCAUCAAAGGGGUCGGCAUGGCAUCUCCACCUCCUCGCGGUC |
| | | CGACCUGGGCAUCCGAAGGAGGACGCACGUCCACUCGGAUGGCUAAG |
| | | GGAGAGCCA |
| 2181 | uvsX, C18G, | GCUGGCGCCUUUAUCUGAUUACUUUGAGAGCCAUCACCAGCGACUAU |
| | trip mut | GUCGUAUGGGUAAAGCGCCCUCUUCGGAGGGCGCAUCAAAG |
| | (T10C), -1 | |
| | A2G, HDV | |
| | AA(98:99)C | |
| 2182 | 3′ HDV | UACUGGCGCUUUUAUCUCAUUACUUUGAGAGCCAUCACCAGCGACUA |
| | ribozyme | UGUCGUAUGGGUAAAGCGCUUAUUUAUCGGAGAGAAAUCCGAUAAAU |
| | (Lior Nissim, | AAGAAGCAUCAAAGUUUUGGCCGGCAUGGUCCCAGCCUCCUCGCUGG |
| | Timothy Lu) | CGCCGGCUGGGCAACAUGCUUCGGCAUGGCGAAUGGGACCCCGGG |
| 2183 | TAC(1:3)GA, | GAUGGCGCCUUUAUCUCAUUACUUUGAGAGCCAUCACCAGCGACUAU |
| | stacked onto | GUCGUAUGGGUAAAGCGCUUACGGACUUCGGUCCGUAAGAAGCAUCA |
| | 64 | AAG |
| 2184 | uvsX, -1 A2G | GCUGGCGCUUUUAUCUCAUUACUUUGAGAGCCAUCACCAGCGACUAU |
| | | GUCGUAUGGGUAAAGCGCCCUCUUCGGAGGGAAGCAUCAAAG |
| 2185 | truncated stem | GCUGGCGCCUUUAUCUGAUUACUUUGAGAGCCAUCACCAGCGACUAU |
| | loop, C18G, | GUCGUAUGGGUAAAGCUCUUACGGACUUCGGUCCGUAAGAGCAUCAA |
| | trip mut | AG |
| | (T10C), -1 | |
| | A2G, HDV | |
| | -99 G65U | |
| 2186 | short phage | GCUGGCGCCUUUAUCUGAUUACUUUGAGAGCCAUCACCAGCGACUAU |
| | rep, C18G, | GUCGUAUGGGUAAAGCUCGGACGACCUCUCGGUCGUCCGAGCAUCAA |
| | trip mut | AG |
| | (T10C), -1 | |
| | A2G, HDV | |
| | -99 G65U | |
| 2187 | 3′ sTRSV WT | UACUGGCGCUUUUAUCUCAUUACUUUGAGAGCCAUCACCAGCGACUA |
| | viral | UGUCGUAUGGGUAAAGCGCUUAUUUAUCGGAGAGAAAUCCGAUAAAU |
| | Hammerhead | AAGAAGCAUCAAAGCCUGUCACCGGAUGUGCUUUCCGGUCUGAUGAG |
| | ribozyme | UCCGUGAGGACGAAACAGG |
| 2188 | short phage | GCUGGCGCUUUUAUCUGAUUACUUUGAGAGCCAUCACCAGCGACUAU |
| | rep, C18G, -1 | GUCGUAUGGGUAAAGCGCGGACGACCUCUCGGUCGUCCGAAGCAUCA |
| | A2G | AAG |
| 2189 | short phage | GCUGGCGCCUUUAUCUGAUUACUUUGAGAGCCAUCACCAGCGACUAU |
| | rep, C18G, | GUCGUAUGGGUAAAGCGCGGACGACCUCUCGGUCGUCCGAAGCAUCA |
| | trip mut | AAG |
| | (T10C), -1 | |
| | A2G, 3′ | |
| | genomic HDV | |
| 2190 | phage rep | GCUGGCGCCUUUAUCUGAUUACUUUGAGAGCCAUCACCAGCGACUAU |
| | loop, C18G, | GUCGUAUGGGUAAAGCUCAGGUGGGACGACCUCUCGGUCGUCCUAUC |
| | trip mut | UGAGCAUCAAAG |
| | (T10C), -1 | |
| | A2G, HDV | |
| | -99 G65U | |
| 2191 | 3′ HDV | UACUGGCGCUUUUAUCUCAUUACUUUGAGAGCCAUCACCAGCGACUA |
| | ribozyme | UGUCGUAUGGGUAAAGCGCUUAUUUAUCGGAGAGAAAUCCGAUAAAU |
| | (Owen Ryan, | AAGAAGCAUCAAAGGAUGGCCGGCAUGGUCCCAGCCUCCUCGCUGGC |
| | Jamie Cate) | GCCGGCUGGGCAACACCUUCGGGUGGCGAAUGGGAC |
| 2192 | phage rep | GCUGGCGCUUUUAUCUGAUUACUUUGAGAGCCAUCACCAGCGACUAU |
| | loop, C18G, -1 | GUCGUAUGGGUAAAGCGCAGGUGGGACGACCUCUCGGUCGUCCUAUC |
| | A2G | UGAAGCAUCAAAG |
| 2193 | 0.14 | UACUGGCGCUUUUAUCUCAUUACUUUGAGAGCCAUCACCAGCGACUA |
| | | UGUCGUACUGGGUAAAGCGCUUAUUUAUCGGAGAGAAAUCCGAUAAA |
| | | UAAGAAGCAUCAAAG |
| 2194 | -78, G77T | UACUGGCGCUUUUAUCUCAUUACUUUGAGAGCCAUCACCAGCGACUA |
| | | UGUCGUAUGGGUAAAGCGCUUAUUUAUCGUGAGAAAUCCGAUAAAUA |
| | | AGAAGCAUCAAAG |
| 2195 | | GUACUGGCGCUUUUAUCUCAUUACUUUGAGAGCCAUCACCAGCGACU |
| | | AUGUCGUAUGGGUAAAGCGCUUAUUUAUCGGAGAGAAAUCCGAUAAA |
| | | UAAGAAGCAUCAAAG |
| 2196 | short phage | GCUGGCGCUUUUAUCUCAUUACUUUGAGAGCCAUCACCAGCGACUAU |
| | rep, -1 A2G | GUCGUAUGGGUAAAGCGCGGACGACCUCUCGGUCGUCCGAAGCAUCA |
| | | AAG |
| 2197 | truncated stem | GCUGGCGCCUUUAUCUGAUUACUUUGAGAGCCAUCACCAGCGACUAU |
| | loop, C18G, | GUCGUAUGGGUAAAGCGCUUACGGACUUCGGUCCGUAAGAAGCAUCA |
| | trip mut | AAG |
| | (T10C), -1 | |
| | A2G | |
| 2198 | -1, A2G | GCUGGCGCUUUUAUCUCAUUACUUUGAGAGCCAUCACCAGCGACUAU |
| | | GUCGUAUGGGUAAAGCGCUUAUUUAUCGGAGAGAAAUCCGAUAAAUA |
| | | AGAAGCAUCAAAG |
| 2199 | truncated stem | GCUGGCGCCUUUAUCUCAUUACUUUGAGAGCCAUCACCAGCGACUAU |
| | loop, trip mut | GUCGUAUGGGUAAAGCGCUUACGGACUUCGGUCCGUAAGAAGCAUCA |
| | (T10C), -1 | AAG |
| | A2G | |
| 2200 | uvsX, C18G, | GCUGGCGCCUUUAUCUGAUUACUUUGAGAGCCAUCACCAGCGACUAU |
| | trip mut | GUCGUAUGGGUAAAGCGCCCUCUUCGGAGGGAAGCAUCAAAG |
| | (T10C), -1 | |
| | A2G | |
| 2201 | phage rep | GCUGGCGCUUUUAUCUCAUUACUUUGAGAGCCAUCACCAGCGACUAU |
| | loop, -1 A2G | GUCGUAUGGGUAAAGCGCAGGUGGGACGACCUCUCGGUCGUCCUAUC |
| | | UGAAGCAUCAAAG |
| 2202 | phage rep | GCUGGCGCCUUUAUCUCAUUACUUUGAGAGCCAUCACCAGCGACUAU |
| | loop, trip mut | GUCGUAUGGGUAAAGCGCAGGUGGGACGACCUCUCGGUCGUCCUAUC |
| | (T10C), -1 | UGAAGCAUCAAAG |
| | A2G | |
| 2203 | phage rep | GCUGGCGCCUUUAUCUGAUUACUUUGAGAGCCAUCACCAGCGACUAU |
| | loop, C18G, | GUCGUAUGGGUAAAGCGCAGGUGGGACGACCUCUCGGUCGUCCUAUC |
| | trip mut | UGAAGCAUCAAAG |
| | (T10C), -1 | |
| | A2G | |
| 2204 | truncated stem | UACUGGCGCUUUUAUCUGAUUACUUUGAGAGCCAUCACCAGCGACUA |
| | loop, C18G | UGUCGUAUGGGUAAAGCGCUUACGGACUUCGGUCCGUAAGAAGCAUC |
| | | AAAG |
| 2205 | uvsX, trip mut | GCUGGCGCCUUUAUCUCAUUACUUUGAGAGCCAUCACCAGCGACUAU |
| | (T10C), -1 | GUCGUAUGGGUAAAGCGCCCUCUUCGGAGGGAAGCAUCAAAG |
| | A2G | |
| 2206 | truncated stem | GCUGGCGCUUUUAUCUCAUUACUUUGAGAGCCAUCACCAGCGACUAU |
| | loop, -1 A2G | GUCGUAUGGGUAAAGCGCUUACGGACUUCGGUCCGUAAGAAGCAUCA |
| | AAG | |
| 2207 | short phage | GCUGGCGCCUUUAUCUCAUUACUUUGAGAGCCAUCACCAGCGACUAU |
| | rep, trip mut | GUCGUAUGGGUAAAGCGCGGACGACCUCUCGGUCGUCCGAAGCAUCA |
| | (T10C), -1 | AAG |
| | A2G | |
| 2208 | 5′HDV | GAUGGCCGGCAUGGUCCCAGCCUCCUCGCUGGCGCCGGCUGGGCAAC |
| | ribozyme | ACCUUCGGGUGGCGAAUGGGACUACUGGCGCUUUUAUCUCAUUACUU |
| | (Owen Ryan, | UGAGAGCCAUCACCAGCGACUAUGUCGUAUGGGUAAAGCGCUUAUUU |
| | Jamie Cate) | AUCGGAGAGAAAUCCGAUAAAUAAGAAGCAUCAAAG |
| 2209 | 5′HDV | GGCCGGCAUGGUCCCAGCCUCCUCGCUGGCGCCGGCUGGGCAACAUU |
| | genomic | CCGAGGGGACCGUCCCCUCGGUAAUGGCGAAUGGGACCCUACUGGCG |
| | ribozyme | CUUUUAUCUCAUUACUUUGAGAGCCAUCACCAGCGACUAUGUCGUAU |
| | | GGGUAAAGCGCUUAUUUAUCGGAGAGAAAUCCGAUAAAUAAGAAGCA |
| | | UCAAAG |
| 2210 | truncated stem | GCUGGCGCCUUUAUCUGAUUACUUUGAGAGCCAUCACCAGCGACUAU |
| | loop, C18G, | GUCGUAUGGGUAAAGCGCUUACGGACUUCGGUCCGUAAGCGCAUCAA |
| | trip mut | AG |
| | (T10C), -1 | |
| | A2G, HDV | |
| | AA(98:99)C | |
| 2211 | 5′env25 pistol | CGUGGUUAGGGCCACGUUAAAUAGUUGCUUAAGCCCUAAGCGUUGAU |
| | ribozyme | CUUCGGAUCAGGUGCAAUACUGGCGCUUUUAUCUCAUUACUUUGAGA |
| | (with an added | GCCAUCACCAGCGACUAUGUCGUAUGGGUAAAGCGCUUAUUUAUCGG |
| | CUUCGG | AGAGAAAUCCGAUAAAUAAGAAGCAUCAAAG |
| | loop) | |
| 2212 | 5′HDV | GGGUCGGCAUGGCAUCUCCACCUCCUCGCGGUCCGACCUGGGCAUCC |
| | antigenomic | GAAGGAGGACGCACGUCCACUCGGAUGGCUAAGGGAGAGCCAUACUG |
| | ribozyme | GCGCUUUUAUCUCAUUACUUUGAGAGCCAUCACCAGCGACUAUGUCG |
| | | UAUGGGUAAAGCGCUUAUUUAUCGGAGAGAAAUCCGAUAAAUAAGAA |
| | | GCAUCAAAG |
| 2213 | 3′ | UACUGGCGCUUUUAUCUCAUUACUUUGAGAGCCAUCACCAGCGACUA |
| | Hammerhead | UGUCGUAUGGGUAAAGCGCUUAUUUAUCGGAGAGAAAUCCGAUAAAU |
| | ribozyme | AAGAAGCAUCAAAGCCAGUACUGAUGAGUCCGUGAGGACGAAACGAG |
| | (Lior Nissim, | UAAGCUCGUCUACUGGCGCUUUUAUCUCAU |
| | Timothy Lu) | |
| | guide scaffold | |
| | scar | |
| 2214 | =+A27, | UACUGGCGCCUUUAUCUCAUUACUUUAGAGAGCCAUCACCAGCGACU |
| | stacked onto | AUGUCGUAUGGGUAAAGCGCUUACGGACUUCGGUCCGUAAGAAGCAU |
| | 64 | CAAAG |
| 2215 | 5′Hammerhead | CGACUACUGAUGAGUCCGUGAGGACGAAACGAGUAAGCUCGUCUAGU |
| | ribozyme | CGUACUGGCGCUUUUAUCUCAUUACUUUGAGAGCCAUCACCAGCGAC |
| | (Lior Nissim, | UAUGUCGUAUGGGUAAAGCGCUUAUUUAUCGGAGAGAAAUCCGAUAA |
| | Timothy Lu) | AUAAGAAGCAUCAAAG |
| | smaller scar | |
| 2216 | phage rep | GCUGGCGCCUUUAUCUGAUUACUUUGAGAGCCAUCACCAGCGACUAU |
| | loop, C18G, | GUCGUAUGGGUAAAGCGCAGGUGGGACGACCUCUCGGUCGUCCUAUC |
| | trip mut | UGCGCAUCAAAG |
| | (T10C), -1 | |
| | A2G, HDV | |
| | AA(98:99)C | |
| 2217 | -27, stacked | UACUGGCGCCUUUAUCUCAUUACUUUAGAGCCAUCACCAGCGACUAU |
| | onto 64 | GUCGUAUGGGUAAAGCGCUUACGGACUUCGGUCCGUAAGAAGCAUCA |
| | | AAG |
| 2218 | 3′ Hatchet | UACUGGCGCUUUUAUCUCAUUACUUUGAGAGCCAUCACCAGCGACUA |
| | | UGUCGUAUGGGUAAAGCGCUUAUUUAUCGGAGAGAAAUCCGAUAAAU |
| | | AAGAAGCAUCAAAGCAUUCCUCAGAAAAUGACAAACCUGUGGGGCGU |
| | | AAGUAGAUCUUCGGAUCUAUGAUCGUGCAGACGUUAAAAUCAGGU |
| 2219 | 3 | UACUGGCGCUUUUAUCUCAUUACUUUGAGAGCCAUCACCAGCGACUA |
| | Hammerhead | UGUCGUAUGGGUAAAGCGCUUAUUUAUCGGAGAGAAAUCCGAUAAAU |
| | ribozyme | AAGAAGCAUCAAAGCGACUACUGAUGAGUCCGUGAGGACGAAACGAG |
| | (Lior Nissim, | UAAGCUCGUCUAGUCGCGUGUAGCGAAGCA |
| | Timothy Lu) | |
| 2220 | 5′Hatchet | CAUUCCUCAGAAAAUGACAAACCUGUGGGGCGUAAGUAGAUCUUCGG |
| | | AUCUAUGAUCGUGCAGACGUUAAAAUCAGGUUACUGGCGCUUUUAUC |
| | | UCAUUACUUUGAGAGCCAUCACCAGCGACUAUGUCGUAUGGGUAAAG |
| | | CGCUUAUUUAUCGGAGAGAAAUCCGAUAAAUAAGAAGCAUCAAAG |
| 2221 | 5′HDV | UUUUGGCCGGCAUGGUCCCAGCCUCCUCGCUGGCGCCGGCUGGGCAA |
| | ribozyme | CAUGCUUCGGCAUGGCGAAUGGGACCCCGGGUACUGGCGCUUUUAUC |
| | (Lior Nissim, | UCAUUACUUUGAGAGCCAUCACCAGCGACUAUGUCGUAUGGGUAAAG |
| | Timothy Lu) | CGCUUAUUUAUCGGAGAGAAAUCCGAUAAAUAAGAAGCAUCAAAG |
| 2222 | 5′Hammerhead | CGACUACUGAUGAGUCCGUGAGGACGAAACGAGUAAGCUCGUCUAGU |
| | ribozyme | CGCGUGUAGCGAAGCAUACUGGCGCUUUUAUCUCAUUACUUUGAGAG |
| | (Lior Nissim, | CCAUCACCAGCGACUAUGUCGUAUGGGUAAAGCGCUUAUUUAUCGGA |
| | Timothy Lu) | GAGAAAUCCGAUAAAUAAGAAGCAUCAAAG |
| 2223 | 3′ HH15 | UACUGGCGCUUUUAUCUCAUUACUUUGAGAGCCAUCACCAGCGACUA |
| | Minimal | UGUCGUAUGGGUAAAGCGCUUAUUUAUCGGAGAGAAAUCCGAUAAAU |
| | Hammerhead | AAGAAGCAUCAAAGGGGAGCCCCGCUGAUGAGGUCGGGGAGACCGAA |
| | ribozyme | AGGGACUUCGGUCCCUACGGGGCUCCC |
| 2224 | 5′ RBMX | CCACCCCCACCACCACCCCCACCCCCACCACCACCCUACUGGCGCUU |
| | recruiting | UUAUCUCAUUACUUUGAGAGCCAUCACCAGCGACUAUGUCGUAUGGG |
| | motif | UAAAGCGCUUAUUUAUCGGAGAGAAAUCCGAUAAAUAAGAAGCAUCA |
| | | AAG |
| 2225 | 3′ | UACUGGCGCUUUUAUCUCAUUACUUUGAGAGCCAUCACCAGCGACUA |
| | Hammerhead | UGUCGUAUGGGUAAAGCGCUUAUUUAUCGGAGAGAAAUCCGAUAAAU |
| | ribozyme | AAGAAGCAUCAAAGCGACUACUGAUGAGUCCGUGAGGACGAAACGAG |
| | (Lior Nissim, | UAAGCUCGUCUAGUCG |
| | Timothy Lu) | |
| | smaller scar | |
| 2226 | 3′ env25 pistol | UACUGGCGCUUUUAUCUCAUUACUUUGAGAGCCAUCACCAGCGACUA |
| | ribozyme | UGUCGUAUGGGUAAAGCGCUUAUUUAUCGGAGAGAAAUCCGAUAAAU |
| | (with an added | AAGAAGCAUCAAAGCGUGGUUAGGGCCACGUUAAAUAGUUGCUUAAG |
| | CUUCGG | CCCUAAGCGUUGAUCUUCGGAUCAGGUGCAA |
| | loop) | |
| 2227 | 3′ Env-9 | UACUGGCGCUUUUAUCUCAUUACUUUGAGAGCCAUCACCAGCGACUA |
| | Twister | UGUCGUAUGGGUAAAGCGCUUAUUUAUCGGAGAGAAAUCCGAUAAAU |
| | | AAGAAGCAUCAAAGGGCAAUAAAGCGGUUACAAGCCCGCAAAAAUAG |
| | | CAGAGUAAUGUCGCGAUAGCGCGGCAUUAAUGCAGCUUUAUUG |
| 2228 | =+ATTATCT | UACUGGCGCUUUUAUCUCAUUACUAUUAUCUCAUUACUUUGAGAGCC |
| | CATTACT25 | AUCACCAGCGACUAUGUCGUAUGGGUAAAGCGCUUAUUUAUCGGAGA |
| | | GAAAUCCGAUAAAUAAGAAGCAUCAAAG |
| 2229 | 5′Env-9 | GGCAAUAAAGCGGUUACAAGCCCGCAAAAAUAGCAGAGUAAUGUCGC |
| | Twister | GAUAGCGCGGCAUUAAUGCAGCUUUAUUGUACUGGCGCUUUUAUCUC |
| | | AUUACUUUGAGAGCCAUCACCAGCGACUAUGUCGUAUGGGUAAAGCG |
| | | CUUAUUUAUCGGAGAGAAAUCCGAUAAAUAAGAAGCAUCAAAG |
| 2230 | 3′ Twisted | UACUGGCGCUUUUAUCUCAUUACUUUGAGAGCCAUCACCAGCGACUA |
| | Sister 1 | UGUCGUAUGGGUAAAGCGCUUAUUUAUCGGAGAGAAAUCCGAUAAAU |
| | | AAGAAGCAUCAAAGACCCGCAAGGCCGACGGCAUCCGCCGCCGCUGG |
| | | UGCAAGUCCAGCCGCCCCUUCGGGGGCGGGCGCUCAUGGGUAAC |
| 2231 | no stem | UACUGGCGCUUUUAUCUCAUUACUUUGAGAGCCAUCACCAGCGACUA |
| | | UGUCGUAUGGGUAAAG |
| 2232 | 5′HH15 | GGGAGCCCCGCUGAUGAGGUCGGGGAGACCGAAAGGGACUUCGGUCC |
| | Minimal | CUACGGGGCUCCCUACUGGCGCUUUUAUCUCAUUACUUUGAGAGCCA |
| | Hammerhead | UCACCAGCGACUAUGUCGUAUGGGUAAAGCGCUUAUUUAUCGGAGAG |
| | ribozyme | AAAUCCGAUAAAUAAGAAGCAUCAAAG |
| 2233 | 5′Hammerhead | CCAGUACUGAUGAGUCCGUGAGGACGAAACGAGUAAGCUCGUCUACU |
| | ribozyme | GGCGCUUUUAUCUCAUUACUGGCGCUUUUAUCUCAUUACUUUGAGAG |
| | (Lior Nissim, | CCAUCACCAGCGACUAUGUCGUAUGGGUAAAGCGCUUAUUUAUCGGA |
| | Timothy Lu) | GAGAAAUCCGAUAAAUAAGAAGCAUCAAAG |
| | guide scaffold | |
| | scar | |
| 2234 | 5′Twisted | ACCCGCAAGGCCGACGGCAUCCGCCGCCGCUGGUGCAAGUCCAGCCG |
| | Sister 1 | CCCCUUCGGGGGCGGGCGCUCAUGGGUAACUACUGGCGCUUUUAUCU |
| | | CAUUACUUUGAGAGCCAUCACCAGCGACUAUGUCGUAUGGGUAAAGC |
| | | GCUUAUUUAUCGGAGAGAAAUCCGAUAAAUAAGAAGCAUCAAAG |
| 2235 | 5′sTRSV WT | CCUGUCACCGGAUGUGCUUUCCGGUCUGAUGAGUCCGUGAGGACGAA |
| | viral | ACAGGUACUGGCGCUUUUAUCUCAUUACUUUGAGAGCCAUCACCAGC |
| | Hammerhead | GACUAUGUCGUAUGGGUAAAGCGCUUAUUUAUCGGAGAGAAAUCCGA |
| | ribozyme | UAAAUAAGAAGCAUCAAAG |
| 2236 | 148, =+G55, | GUACUGGCGCCUUUAUCUCAUUACUUUGAGAGCCAUCACCAGCGACU |
| | stacked onto | AUGUCGUAGUGGGUAAAGCGCUUACGGACUUCGGUCCGUAAGAAGCA |
| | 64 | UCAAAG |
| 2237 | 158, | GUACUGGCGCUUUUAUCUGAUUACUUUGAGAGCCAUCACCAGCGACU |
| | 103 + 148 (+G55) | AUGUCGUAGUGGGUAAAGCUCCCUCUUCGGAGGGAGCAUCAAAG |
| | -99, G65U | |
| 2238 | 174, Uvsx | ACUGGCGCUUUUAUCUGAUUACUUUGAGAGCCAUCACCAGCGACUAU |
| | Extended stem | GUCGUAGUGGGUAAAGCUCCCUCUUCGGAGGGAGCAUCAAAG |
| | with [A99] | |
| | G65U), | |
| | C18G, \hat{ }G55, | |
| | [GT-1] | |
| 2239 | 175, extended | ACUGGCGCCUUUAUCUCAUUACUUUGAGAGCCAUCACCAGCGACUAU |
| | stem | GUCGUAUGGGUAAAGCGCUUACGGACUUCGGUCCGUAAGAAGCAUCA |
| | truncation, | AAG |
| | T10C, [GT-1] | |
| 2240 | 176, 174 with | GCUGGCGCUUUUAUCUGAUUACUUUGAGAGCCAUCACCAGCGACUAU |
| | A1G | GUCGUAGUGGGUAAAGCUCCCUCUUCGGAGGGAGCAUCAAAG |
| | substitution | |
| | for T7 | |
| | transcription | |
| 2241 | 177, 174 with | ACUGGCGCUUUUAUCUGAUUACUUUGAGAGCCAUCACCAGCGACUAU |
| | bubble (+G55) | GUCGUAUGGGUAAAGCUCCCUCUUCGGAGGGAGCAUCAAAG |
| | removed | |
| 2242 | 181, stem 42 | ACUGGCGCCUUUAUCUGAUUACUUUGAGAGCCAUCACCAGCGACUAU |
| | (truncated | GUCGUAUGGGUAAAGCGCUUACGGACUUCGGUCCGUAAGAAGCAUCA |
| | stem loop); | AAG |
| | T10C, C18G, | |
| | [GT-1] | |
| | (95+[GT-1]) | |
| 2243 | 182, stem 42 | ACUGGCGCUUUUAUCUGAUUACUUUGAGAGCCAUCACCAGCGACUAU |
| | (truncated | GUCGUAUGGGUAAAGCGCUUACGGACUUCGGUCCGUAAGAAGCAUCA |
| | stem loop); | AAG |
| | C18G, [GT-1] | |
| 2244 | 183, stem 42 | ACUGGCGCUUUUAUCUGAUUACUUUGAGAGCCAUCACCAGCGACUAU |
| | (truncated | GUCGUAGUGGGUAAAGCGCUUACGGACUUCGGUCCGUAAGAAGCAUC |
| | stem loop); | AAAG |
| | C18G, \hat{ }G55, | |
| | [GT-1] | |
| 2245 | 184, stem 48 | ACUGGCGCUUUUAUCUGAUUACUUUGAGAGCCAUCACCAGCGACUAU |
| | (uvsx, -99 | GUCGUAUUGGGUAAAGCUCCCUCUUCGGAGGGAGCAUCAAAG |
| | g65t); | |
| | C18G, \hat{ }T55, | |
| | [GT-1] | |
| 2246 | 185, stem 42 | ACUGGCGCUUUUAUCUGAUUACUUUGAGAGCCAUCACCAGCGACUAU |
| | (truncated | GUCGUAUUGGGUAAAGCGCUUACGGACUUCGGUCCGUAAGAAGCAUC |
| | stem loop); | AAAG |
| | C18G, \hat{ }T55, | |
| | [GT-1] | |
| 2247 | 186, stem 42 | ACUGGCGCCUUUAUCAUCAUUACUUUGAGAGCCAUCACCAGCGACUA |
| | (truncated | UGUCGUAUGGGUAAAGCGCUUACGGACUUCGGUCCGUAAGAAGCAUC |
| | stem loop); | AAAG |
| | T10C, \hat{ }A17, | |
| | [GT-1] | |
| 2248 | 187, stem 46 | ACUGGCGCUUUUAUCUGAUUACUUUGAGAGCCAUCACCAGCGACUAU |
| | (uvsx); | GUCGUAGUGGGUAAAGCGCCCUCUUCGGAGGGAAGCAUCAAAG |
| | C18G, \hat{ }G55, | |
| | [GT-1] | |
| 2249 | 188, stem 50 | ACUGGCGCUUUUAUCUGAUUACUUUGAGAGCCAUCACCAGCGACUAU |
| | (ms2 U15C, | GUCGUAGUGGGUAAAGCUCACAUGAGGAUCACCCAUGUGAGCAUCAA |
| | -99, g65t); | AG |
| | C18G, \hat{ }G55, | |
| | [GT-1] | |
| 2250 | 189, 174 + | ACUGGCACUUUUACCUGAUUACUUUGAGAGCCAACACCAGCGACUAU |
| | G8A; T15C; | GUCGUAGUGGGUAAAGCUCCCUCUUCGGAGGGAGCAUCAAAG |
| | T35A | |
| 2251 | 190, 174 + | ACUGGCACUUUUAUCUGAUUACUUUGAGAGCCAUCACCAGCGACUAU |
| | G8A | GUCGUAGUGGGUAAAGCUCCCUCUUCGGAGGGAGCAUCAAAG |
| 2252 | 191, 174 + | ACUGGCCCUUUUAUCUGAUUACUUUGAGAGCCAUCACCAGCGACUAU |
| | G8C | GUCGUAGUGGGUAAAGCUCCCUCUUCGGAGGGAGCAUCAAAG |
| 2253 | 192, 174 + | ACUGGCGCUUUUACCUGAUUACUUUGAGAGCCAUCACCAGCGACUAU |
| | T15C | GUCGUAGUGGGUAAAGCUCCCUCUUCGGAGGGAGCAUCAAAG |
| 2254 | 193, 174 + | ACUGGCGCUUUUAUCUGAUUACUUUGAGAGCCAACACCAGCGACUAU |
| | 135A | GUCGUAGUGGGUAAAGCUCCCUCUUCGGAGGGAGCAUCAAAG |
| 2255 | 195, 175 + | ACUGGCACCUUUACCUGAUUACUUUGAGAGCCAACACCAGCGACUAU |
| | C18G + | GUCGUAUGGGUAAAGCGCUUACGGACUUCGGUCCGUAAGAAGCAUCA |
| | G8A; T15C; | AAG |
| | T35A | |
| 2256 | 196, 175+ | ACUGGCACCUUUAUCUGAUUACUUUGAGAGCCAUCACCAGCGACUAU |
| | C18G + G8A | GUCGUAUGGGUAAAGCGCUUACGGACUUCGGUCCGUAAGAAGCAUCA |
| | | AAG |
| 2257 | 197, 175 + | ACUGGCCCCUUUAUCUGAUUACUUUGAGAGCCAUCACCAGCGACUAU |
| | C18G + G8C | GUCGUAUGGGUAAAGCGCUUACGGACUUCGGUCCGUAAGAAGCAUCA |
| | | AAG |
| 2258 | 198, 175 + | ACUGGCGCCUUUAUCUGAUUACUUUGAGAGCCAACACCAGCGACUAU |
| | C18G +T35A | GUCGUAUGGGUAAAGCGCUUACGGACUUCGGUCCGUAAGAAGCAUCA |
| | | AAG |
| 2259 | 199, 174 + | GCUGGCGCUUUUAUCUGAUUACUUUGAGAGCCAUCACCAGCGACUAU |
| | A2G (test G | GUCGUAGUGGGUAAAGCUCCCUCUUCGGAGGGAGCAUCAAAG |
| | transcription | |
| | at start; | |
| | ccGCT...) | |
| 2260 | 200, 174 + | GACUGGCGCUUUUAUCUGAUUACUUUGAGAGCCAUCACCAGCGACUA |
| | \hat{ }G1 | UGUCGUAGUGGGUAAAGCUCCCUCUUCGGAGGGAGCAUCAAAG |
| | (ccGACT...) | |
| 2261 | 201, 174 + | ACUGGCGCCUUUAUCUGAUUACUUUGGAGAGCCAUCACCAGCGACUA |
| | T10C; \hat{ }G28 | UGUCGUAGUGGGUAAAGCUCCCUCUUCGGAGGGAGCAUCAAAG |
| 2262 | 202, 174 + | ACUGGCGCAUUUAUCUGAUUACUUUGUGAGCCAUCACCAGCGACUAU |
| | T10A; \hat{ }28T | GUCGUAGUGGGUAAAGCUCCCUCUUCGGAGGGAGCAUCAAAG |
| 2263 | 203, 174 + | ACUGGCGCCUUUAUCUGAUUACUUUGAGAGCCAUCACCAGCGACUAU |
| | T10C | GUCGUAGUGGGUAAAGCUCCCUCUUCGGAGGGAGCAUCAAAG |
| 2264 | 204,174+ | ACUGGCGCUUUUAUCUGAUUACUUUGGAGAGCCAUCACCAGCGACUA |
| | \hat{ }G28 | UGUCGUAGUGGGUAAAGCUCCCUCUUCGGAGGGAGCAUCAAAG |
| 2265 | 205, 174 + | ACUGGCGCAUUUAUCUGAUUACUUUGAGAGCCAUCACCAGCGACUAU |
| | T10A | GUCGUAGUGGGUAAAGCUCCCUCUUCGGAGGGAGCAUCAAAG |
| 2266 | 206, 174 + | ACUGGCGCUUUUAUCUGAUUACUUUGUGAGCCAUCACCAGCGACUAU |
| | A28T | GUCGUAGUGGGUAAAGCUCCCUCUUCGGAGGGAGCAUCAAAG |
| 2267 | 207, 174+ | ACUGGCGCUUUUAUUCUGAUUACUUUGAGAGCCAUCACCAGCGACUA |
| | \hat{ }T15 | UGUCGUAGUGGGUAAAGCUCCCUCUUCGGAGGGAGCAUCAAAG |
| 2268 | 208, 174 + | ACGGCGCUUUUAUCUGAUUACUUUGAGAGCCAUCACCAGCGACUAUG |
| | [T4] | UCGUAGUGGGUAAAGCUCCCUCUUCGGAGGGAGCAUCAAAG |
| 2269 | 209,174+ | ACUGGCGCUUUUAUAUGAUUACUUUGAGAGCCAUCACCAGCGACUAU |
| | C16A | GUCGUAGUGGGUAAAGCUCCCUCUUCGGAGGGAGCAUCAAAG |
| 2270 | 210, 174 + | ACUGGCGCUUUUAUCUUGAUUACUUUGAGAGCCAUCACCAGCGACUA |
| | \hat{ }T17 | UGUCGUAGUGGGUAAAGCUCCCUCUUCGGAGGGAGCAUCAAAG |
| 2271 | 211, 174 + | ACUGGCGCUUUUAUCUGAUUACUUUGAGAGCCAGCACCAGCGACUAU |
| | T35G | GUCGUAGUGGGUAAAGCUCCCUCUUCGGAGGGAGCAUCAAAG |
| | (compare with | |
| | 174 + T35A | |
| | above) | |
| 2272 | 212, 174 + | ACUGGCGCUGUUAUCUGAUUACUUCGAGAGCCAUCACCAGCGACUAU |
| | U11G, | GUCGUAGUGGGUAAAGCUCCCUCUUCGGAGGGAGCAUCGAAG |
| | A105G | |
| | (A86G), | |
| | U26C | |
| 2273 | 213, 174 + | ACUGGCGCUCUUAUCUGAUUACUUCGAGAGCCAUCACCAGCGACUAU |
| | U11C, | GUCGUAGUGGGUAAAGCUCCCUCUUCGGAGGGAGCAUCGAAG |
| | A105G | |
| | (A86G), | |
| | U26C | |
| 2274 | 214, | ACUGGCGCUUGUAUCUGAUUACUCUGAGAGCCAUCACCAGCGACUAU |
| | 174 + U12G; | GUCGUAGUGGGUAAAGCUCCCUCUUCGGAGGGAGCAUCAGAG |
| | A106G | |
| | (A87G), | |
| | U25C | |
| 2275 | 215, 174 + U12C; | ACUGGCGCUUCUAUCUGAUUACUCUGAGAGCCAUCACCAGCGACUAU |
| | A106G | GUCGUAGUGGGUAAAGCUCCCUCUUCGGAGGGAGCAUCAGAG |
| | (A87G), | |
| | U25C | |
| 2276 | 216, | ACUGGCGCUUUGAUCUGAUUACCUUGAGAGCCAUCACCAGCGACUAU |
| | 174_tx_11.G, | GUCGUAGUGGGUAAAGCUCCCUCUUCGGAGGGAGCAUCAAGG |
| | 87.G, 22.C | |
| 2277 | 217, | ACUGGCGCUUUCAUCUGAUUACCUUGAGAGCCAUCACCAGCGACUAU |
| | 174_tx_11.C, | GUCGUAGUGGGUAAAGCUCCCUCUUCGGAGGGAGCAUCAAGG |
| | 87.G, 22.C | |
| 2278 | 218, 174 + | ACUGGCGCUGUUAUCUGAUUACUUUGAGAGCCAUCACCAGCGACUAU |
| | I11G | GUCGUAGUGGGUAAAGCUCCCUCUUCGGAGGGAGCAUCAAAG |
| 2279 | 219, 174 + | ACUGGCGCUUUUAUCUGAUUACUUUGAGAGCCAUCACCAGCGACUAU |
| | A105G | GUCGUAGUGGGUAAAGCUCCCUCUUCGGAGGGAGCAUCGAAG |
| | (A86G) | |
| 2280 | 220, 174 + | ACUGGCGCUUUUAUCUGAUUACUUCGAGAGCCAUCACCAGCGACUAU |
| | U26C | GUCGUAGUGGGUAAAGCUCCCUCUUCGGAGGGAGCAUCAAAG |

VI. Methods of Constructing the Library

The libraries described herein may be constructed in a variety of ways. Libraries may be constructed using, for example PCR-based mutagenesis, plasmid recombineering, or other methods known to one of skill in the art to generate protein and RNA variants. In some embodiments, a combination of methods are used to construct one or more variant libraries.

In some embodiments, PCR-based mutagenesis is used to construct variant RNA libraries, such as sgRNA variant libraries. For example, in some embodiments, a PCR mutagenesis method using degenerate oligonucleotides is used to produce single nucleotide substitution variants. These degenerate oligonucleotides may be synthesized such that each locus of the primer that is complementary to the sgRNA locus has a 97% chance of being the wild type base, and a 1% chance of being each of the other three naturally occurring nucleotides. During PCR, the degenerate oligos may anneal to, and just beyond, the sgRNA scaffold within a small plasmid, amplifying the entire plasmid. The PCR product can then be purified, ligated, and transformed into a cell, such as E. coli, for screening. In other embodiments, a different PCR method is used to construct sgRNA scaffolds with single nucleotide insertions and deletions. For example, a unique PCR reaction is set up for each base pair intended for mutation. These PCR primers can be designed and paired such that PCR products will either be missing a base pair, or contain an additional inserted base pair. For inserted base pairs, PCR primers will insert a degenerate base such that all four possible naturally occurring nucleotides are represented in the final library.

In some embodiments of the DME methods provided herein, mutations are incorporated into double stranded DNA encoding the biomolecule. This DNA can be maintained and replicated in a standard cloning vector, for example a bacterial plasmid, referred to herein as the target plasmid. In some embodiments, an exemplary target plasmid contains a DNA sequence encoding the reference biomolecule that will be subjected to DME, a bacterial origin of replication, and a suitable antibiotic resistance expression cassette. In some embodiments, the antibiotic resistance cassette confers resistance to Kanamycin, Ampicillin, Spectinomycin, Bleomycin, Streptomycin, Erythromycin, Tetracycline, or Chloramphenicol. In some embodiments, the antibiotic resistance cassette confers resistance to Kanamycin.

Thus, in some embodiments, provided herein is a method of constructing a library of polynucleotide variants of a reference biomolecule, comprising:
(a) constructing a polynucleotide that encodes for a variant of the reference biomolecule, wherein the reference biomolecule is a protein or RNA or DNA;
   wherein the polynucleotide encodes an alteration of one or more monomer locations of the reference biomolecule, wherein the monomer is an amino acid of the protein or ribonucleotide of the RNA or deoxyribonucleotide of DNA, and
   wherein each alteration of a monomer location is independently selected from the group consisting of substitution of the monomer, deletion of one or more consecutive monomers beginning at the location, and insertion of one or more consecutive monomers adjacent to the location; and
(b) repeating the polynucleotide construction of (a) a sufficient number of times such that the library of polynucleotide represents variants comprising a single alteration of a single location for at least 1% of the monomer locations of the biomolecule.

Said methods of polynucleotide library construction may be used to produce a polynucleotide library representing any of the variant libraries described herein. For example, such methods may be used to construct a library of polynucleotides representing variants comprising a single alteration of a single location for at least 5%, at least 10%, at least 30%, at least 70%, at least 90%, or any other % described herein of the total monomer locations of the reference biomolecule; or variants comprising substitution of the monomer, variants comprising deletion of one or more monomers beginning at the location, and variants comprising insertion of one or more new monomers adjacent to the location for at least 1%, at least 5%, at least 10%, at least 30%, at least 50%, at least 70%, at least 90%, or other % of monomer locations; and wherein insertion comprises insertion of one to four monomers; or deletion comprises deletion of one to four monomers; or substitution comprises substitution with each of the other naturally occurring monomers; or variants each independently comprising alteration of one, two, three, four, five, six, seven, eight, nine, ten, eleven, twelve, thirteen, fourteen, fifteen, sixteen, seventeen, eighteen, nineteen, twenty, or more locations, wherein the library as a whole represents alteration of at least 5%, at least 10%, at least 30%, at least 70%, or at least 90% of the total locations of the reference biomolecule; or any combinations thereof, or any other variant libraries described herein. In some embodiments, each variant biomolecule independently comprises alteration of between one to twenty, between one to ten, between one to five, between five to ten, between five to fifteen, between five to twenty, between ten to fifteen, between ten to twenty, between fifteen to twenty, or between three to seven, or between three to ten monomer locations.

A library comprising said variants can be constructed in a variety of ways. In certain embodiments, plasmid recombineering is used to construct a library. Such methods can use DNA oligonucleotides encoding one or more mutations to incorporate said mutations into a plasmid encoding the reference biomolecule. For biomolecule variants with a plurality of mutations, in some embodiments more than one oligonucleotide is used. In some embodiments, the DNA oligonucleotides encoding one or more mutations wherein the mutation region is flanked by between 10 and 100 nucleotides of homology to the target plasmid, both 5′ and 3′ to the mutation. Such oligonucleotides can in some embodiments be commercially synthesized and used in PCR amplification. An exemplary template for an oligonucleotide encoding a mutation is provided below
5′-(N)₁₀₋₁₀₀−Mutation−(N′)₁₀₋₁₀₀−3′
   wherein the region encoding the mutation is flanked on the 5′ and 3′ ends by between 10 to 100 (independently) nucleotides that are homologous to the target plasmid (e.g., “homology arms”). The region encoding the desired mutation or mutations will comprise three nucleotides encoding an amino acid (for substitutions or single insertions), or zero nucleotides (for deletions). In some embodiments the oligonucleotide encodes insertion of greater than one amino acid. For example, wherein the oligonucleotide encodes the insertion of X amino acids, the region encoding the desired mutation comprises 3*X nucleotides encoding the X amino acids. In some embodiments, the mutation region encodes more than one mutation, for example mutations to two or more monomers of a biomolecule that are in close proximity (e.g., next to each other, or within 1, 2, 3, 4, 5, 6, 7, 8, 9, or 10, or more monomers of each other).

Such exemplary oligonucleotides may, for example, encode protein variants or RNA variants. For example, wherein the reference biomolecule is a protein, 40 different amino acid mutations to a single monomer in a protein can be encoded using 40 different oligonucleotides comprising the same set of homology arms (e.g., substitution with each of the 19 other naturally occurring amino acids, single insertion of each of the 20 naturally occurring amino acids, and single deletion of the original amino acid). In some embodiments, wherein the reference biomolecule is RNA, 8 possible oligonucleotides, using one set of homology arms, can be used to encode the 8 different nucleotide mutations to a single monomer (e.g., substitution with each of the other three naturally occurring nucleotides, single insertion of each of the 4 naturally occurring nucleotides, and single deletion of the original nucleotide). In some embodiments, wherein one or more non-natural monomers is used, additional oligonucleotides are constructed. In some embodiments, different pairs of homology arms (e.g., pairs of homology arms of different lengths) can be used to encode variants of the same target monomer or monomers.

Nucleotide sequences code for particular amino acid monomers in a substitution or insertion mutation in an oligo as described herein will be known to the person of ordinary skill in the art. For example, TTT or TTC triplets can be used to encode phenylalanine; TTA, TTG, CTT, CTC, CTA or CTG can be used to encode leucine; ATT, ATC or ATA can be used to encode isoleucine; ATG can be used to encode methionine; GTT, GTC, GTA or GTG c can be used to encode valine; TCT, TCC, TCA, TCG, AGT or AGC can be used to encode serine; CCT, CCC, CCA or CCG can be used to encode proline; ACT, ACC, ACA or ACG can be used to encode threonine; GCT, GCC, GCA or GCG can be used to encode alanine; TAT or TAC can be used to encode tyrosine; CAT or CAC can be used to encode histidine; CAA or CAG can be used to encode glutamine, AAT or AAC can be used to encode asparagine; AAA or AAG can be used to encode lysine; GAT or GAC can be used to encode aspartic acid; GAA or GAG can be used to encode glutamic acid; TGT or TGC c can be used to encode cysteine; TGG can be used to encode tryptophan; CGT, CGC, CGA, CGG, AGA or AGG can be used to encode arginine; and GGT, GGC, GGA or GGG can be used to encode glycine. In addition, ATG is used for initiation of the peptide synthesis as well as for methionine and TAA, TAG and TGA can be used to encode for the termination of the peptide synthesis.

In some exemplary embodiments where the reference biomolecule undergoing DME is an RNA, 8 different oligonucleotides, using the same set of homology arms, encode the above enumerated 8 different single nucleotide mutations for each nucleotide in the RNA that is targeted for DME. When the mutation is of a single ribonucleotide, the region of the oligo encoding the mutations can consist of the following nucleotide sequences: one nucleotide specifying a nucleotide (for substitutions or insertions), or zero nucleotides (for deletions). In some embodiments, the oligonucleotides are synthesized as single stranded DNA oligonucleotides. In some embodiments, all oligonucleotides targeting a particular amino acid or nucleotide of a biomolecule subjected to DME are pooled. In some embodiments, all oligonucleotides targeting a biomolecule subjected to DME are pooled. There is no limit to the type or number of mutations that can be created simultaneously in a library.

Therefore, in some aspects, provided herein is a library of variant oligonucleotides, wherein:
each variant oligonucleotide independently encodes an alteration of one or more sequential monomer locations of a reference biomolecule, wherein:
the reference biomolecule is a protein, RNA, or DNA,
the one or more monomers are one or more amino acids of the protein or ribonucleotides of the RNA or deoxyribonucleotide of the DNA, and
wherein each alteration of a monomer location is independently selected from the group consisting of substitution of the monomer, deletion of one or more consecutive monomers beginning at the location, and insertion of one or more consecutive monomers adjacent to the location;
each variant oligonucleotide comprises a pair of homology arms flanking the encoded alteration, wherein the homology arms are homologous to the reference biomolecule sequences flanking the corresponding monomer location alteration, and wherein each homology arm independently comprises between 10 to 100 nucleotides; and
the library of variant oligonucleotides represents alteration of a single monomer for at least 1% of monomer locations.

In some embodiments, the library of variant oligonucleotides represents alteration of a single monomer for at least 5%, at least 10%, at least 20%, at least 30%, at least 40%, at least 50%, at least 60%, at least 70%, at least 80%, at least 90%, at least 95%, at least 99%, or 100% of monomer locations. In certain embodiments, the library of variant oligonucleotides represents alteration of a single monomer for between 10% to 100%, between 20% to 100%, between 30% to 100%, between 40% to 100%, between 50% to 100%, between 60% to 100%, between 70% to 100%, between 80% to 100, or between 90% to 100% of monomer locations. In some embodiments, the library of variant oligonucleotides represents a library of variant biomolecules, wherein each variant biomolecule independently comprises alteration of one, two, three, four, five, six, seven, eight, nine, ten, eleven, twelve, thirteen, fourteen, fifteen, sixteen, seventeen, eighteen, nineteen, twenty or more locations, wherein the library as a whole represents alteration of at least 5%, at least 10%, at least 30%, at least 70%, or at least 90% of the total locations of the reference biomolecule. In some embodiments, the library of variant oligonucleotides represents a library of variant biomolecules, wherein each variant biomolecule independently comprises alteration of between one to twenty, between one to ten, between one to five, between five to ten, between five to fifteen, between five to twenty, between ten to fifteen, between ten to twenty, between fifteen to twenty, or between three to seven, or between three to ten monomer locations.

Plasmid recombineering can then be used to recombine these synthetic mutations into a target gene of interest. In some embodiments of plasmid recombineering methods, a target plasmid encoding the reference protein, a standard bacterial origin of replication, and an antibiotic resistance cassette (e.g., an antibiotic resistance cassette conferring resistance to Kanamycin, Ampicillin, Spectinomycin, Bleomycin, Streptomycin, Erythromycin, Tetracycline, or Chloramphenicol) is constructed. A library of oligonucleotides encoding the desired mutation may be constructed, for example, through commercial synthesis. A plurality of plasmids and the library of oligonucleotides are combined and introduced into an expression cell, for example introduced into E. coli (such as EcNR2 cells) using electroporation. The electroporated cells are then grown in the presence of the antibiotic, selecting for cells that have been transformed with the plasmid. Plasmids from these transformed cells are isolated using standard methods known to one of skill in the art, resulting in a plurality of plasmids, into at least some of which an oligonucleotide encoding for the desired mutation has been incorporated. Thus, at least a portion of the plasmids encode for protein variants. The isolated plasmids may also include plasmids that encode the reference protein, without incorporating any mutations. For example, in some embodiments, a single round of plasmid recombineering may produce a plurality of plasmids in which 10-30% independently encode for protein variants. Performing another round of plasmid recombineering using the plurality of isolated plasmids with another library of oligonucleotides (either the same library or a new library) may, in some embodiments, increase the total percentage of plasmids that encode for a protein variant. In certain embodiments, performing additional rounds of plasmid recombineering using plasmids from the previous round also results in stacking of mutations, for example producing plasmids that encode for variants comprising two, three, four, five, or more monomer alterations.

Therefore, in some aspects, provided herein is a vector library comprising a plurality of vectors, wherein each vector independently comprises one variant oligonucleotide of an oligonucleotide library as described herein. In certain embodiments, the vectors are constructed using plasmid recombineering. Exemplary vectors may include, but are not limited to, lentiviral vectors, adenoviral vectors, adeno-associated viral (AAV) vectors, and bacterial plasmids. In some embodiments, the vector is a bacterial plasmid further comprising a bacterial origin of replication and an antibiotic resistance expression cassette (e.g., conferring resistance to Kanamycin, Ampicillin, Spectinomycin, Bleomycin, Streptomycin, Erythromycin, Tetracycline or Chloramphenicol).

Further provided are methods of selecting a biomolecule variant, comprising producing a library of reference biomolecule variants from a polynucleotide variant library as described herein, or a vector library as described herein; screening the library of biomolecule variants for one or more functional characteristics; and selecting a biomolecule variant from the library.

In some embodiments, for certain libraries, methods of plasmid recombineering must be altered. For example, for some libraries, additional rounds plasmid recombineering are needed to construct enough vectors of sufficient diversity to adequately sample the desired alteration space of the reference molecule (e.g., 2, 3, 4, 5, 6, 7, 8, 9, 10, 11, or more rounds). In certain embodiments, a higher concentration of oligos encoding the alterations must be combined with the plasmid vectors to construct enough vectors of sufficient diversity to adequately sample the desired alteration space of the reference molecule. In some variations, the number of additional rounds and/or increased concentration of oligos does not have a linear relationship with the increased sampling space needed. Certain parameters may therefore be affected by reference biomolecule size and/or level of desired diversity in the library, but cannot be derived directly in a linear relationship in some embodiments.

In other embodiments, methods other than plasmid recombineering are used to construct one or more DME libraries, or a combination of plasmid recombineering and other methods are used to construct one or more DME libraries. For example, DME libraries may, in some embodiments, be constructed using one of the other mutational methods described herein. Such libraries may then be taken through the library screening as described herein, and further iterations be carried out if desired.

Collectively, the methods of the disclosure result in variants of CasX proteins and guides that can form ribonucleoprotein complexes (RNP), or gene editing pairs, that, in some embodiments, have one or more improved characteristics compared to a gene editing pair of a reference CasX and reference guide RNA. Exemplary improved characteristics, as described herein, may in some embodiments, and include improved CasX:gNA RNP complex stability, improved binding affinity between the CasX and gNA, improved kinetics of RNP complex formation, higher percentage of cleavage-competent RNP, improved RNP binding affinity to the target DNA, improved unwinding of the target DNA, increased editing activity, improved editing efficiency, improved editing specificity, increased activity of the nuclease, increased target strand loading for double strand cleavage, decreased target strand loading for single strand nicking, decreased off-target cleavage, improved binding of the non-target strand of DNA, or improved resistance to nuclease activity. In the foregoing embodiments, the improvement is at least about 2-fold, at least about 5-fold, at least about 10-fold, at least about 50-fold, at least about 100-fold, at least about 500-fold, at least about 1000-fold, at least about 5000-fold, at least about 10,000-fold, or at least about 100,000-fold compared to the characteristic of a reference CasX protein and reference gNA pair. In other cases, the one or more of the improved characteristics may be improved about 1.1 to 100,00×, about 1.1 to 10,00×, about 1.1 to 1,000×, about 1.1 to 500×, about 1.1 to 100×, about 1.1 to 50×, about 1.1 to 20×, about 10 to 100,00×, about 10 to 10,00×, about 10 to 1,000×, about 10 to 500×, about 10 to 100×, about 10 to 50×, about 10 to 20×, about 2 to 70×, about 2 to 50×, about 2 to 30×, about 2 to 20×, about 2 to 10×, about 5 to 50×, about 5 to 30×, about 5 to 10×, about 100 to 100,00×, about 100 to 10,00×, about 100 to 1,000×, about 100 to 500×, about 500 to 100,00×, about 500 to 10,00×, about 500 to 1,000×, about 500 to 750×, about 1,000 to 100,00×, about 10,000 to 100,00×, about 20 to 500×, about 20 to 250×, about 20 to 200×, about 20 to 100×, about 20 to 50×, about 50 to 10,000×, about 50 to 1,000×, about 50 to 500×, about 50 to 200×, or about 50 to 100×, improved relative to a reference gene editing pair. In other cases, the one or more of the improved characteristics may be improved about 1.1×, 1.2×, 1.3×, 1.4×, 1.5×, 1.6×, 1.7×, 1.8×, 1.9×, 2×, 3×, 4×, 5×, 6×, 7×, 8×, 9×, 10×, 11×, 12×, 13×, 14×, 15×, 16×, 17×, 18×, 19×, 20×, 25×, 30×, 40×, 45×, 50×, 55×, 60×, 70×, 80×, 90×, 100×, 110×, 120×, 130×, 140×, 150×, 160×, 170×, 180×, 190×, 200×, 210×, 220×, 230×, 240×, 250×, 260×, 270×, 280×, 290×, 300×, 310×, 320×, 330×, 340×, 350×, 360×, 370×, 380×, 390×, 400×, 425×, 450×, 475×, or 500× improved relative to a reference gene editing pair. In some embodiments, the variant gene editing pair comprises a gNA variant comprising a sequence of any one of SEQ ID NOs: 2101-2280 and a CasX variant of Table 1. In some embodiments, the gene editing pair comprises a CasX selected from any one of CasX 119, CasX 438, CasX 457, CasX 488, or CasX 491 and a gNA selected from any one of SEQ ID NOS: 2104, 2106, or 2238.

The description herein sets forth numerous exemplary configurations, methods, parameters, and the like. It should be recognized, however, that such description is not intended as a limitation on the scope of the present disclosure, but is instead provided as a description of exemplary embodiments.

VII. Kits and Articles of Manufacture

In some aspects, provided herein are kits comprising a biomolecule protein variant as described herein and a suitable container (for example a tube, vial or plate).

In some embodiments, the biomolecule variant is a Cas protein variant (such as a CasX variant protein). In some embodiments, the biomolecule variant is a CasX variant protein, and the kit further comprises a CasX guide RNA variant as described herein, or the reference guide RNA of SEQ ID NO: 4 or SEQ ID NO: 5.

In other embodiments, the biomolecule variant is a gRNA variant (such as a gRNA variant that binds to CasX). In some embodiments, the biomolecule variant is a CasX gRNA variant and the kit further comprises a CasX variant protein as described herein, or the reference CasX protein of SEQ ID NO: 1, SEQ ID NO: 2, or SEQ ID NO: 3.

In certain embodiments, provided herein are kits comprising a CasX protein and gRNA pair comprising a CasX variant protein and a CasX gRNA variant as described herein.

In some embodiments, the kit further comprises a buffer, a nuclease inhibitor, a protease inhibitor, a liposome, a therapeutic agent, a label, a label visualization reagent, or any combination of the foregoing. In some embodiments, the kit further comprises a pharmaceutically acceptable carrier, diluent or excipient.

In some embodiments, the kit comprises appropriate control compositions for gene editing applications, and instructions for use.

In some embodiments, the kit comprises a vector comprising a sequence encoding a CasX variant protein of the disclosure, a CasX gRNA variant of the disclosure, or a combination thereof.

### EXAMPLES

The following Examples are merely illustrative and are not meant to limit any aspects of the present disclosure in any way.

### Example 1: Assays Used to Measure sgRNA and CasX Protein Activity

Several assays were used to carry out initial screens of CasX protein and sgRNA DME libraries and engineered mutants, and to measure the activity of select protein and sgRNA variants relative to CasX reference sgRNAs and proteins.

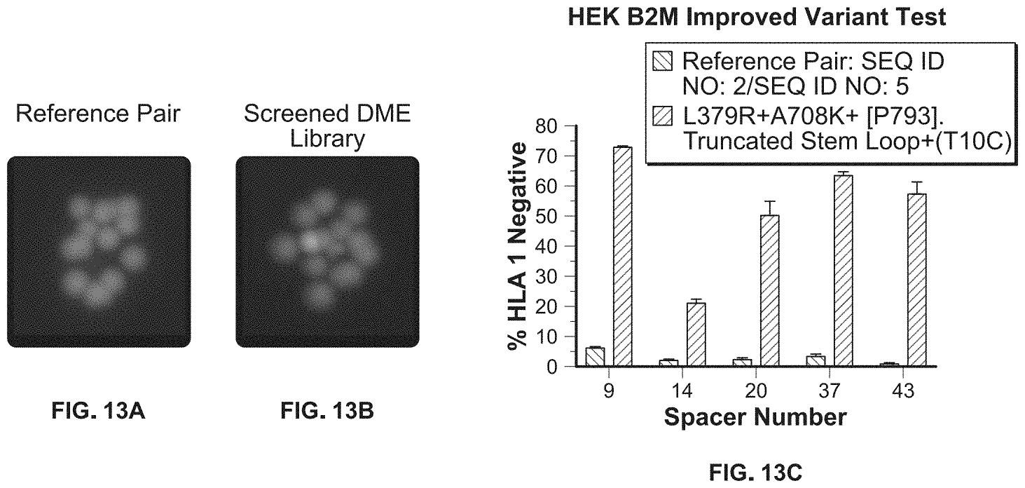

E. coli CRISPRi screen: Briefly, biological triplicates of dead CasX DME Libraries on a chloramphenicol (CM) resistant plasmid with a GFP guide RNA on a carbenicillin (Carb) resistant plasmid were transformed (at >5× library size) into MG1655 with genetically integrated and constitutively expressed GFP and RFP (see FIG. 13A-13B). Cells were grown overnight in EZ-RDM+Carb, CM and Anhydrotetracycline (aTc) inducer. E. coli were FACS sorted based on gates for the top 1% of GFP but not RFP repression, collected, and resorted immediately to further enrich for highly functional CasX molecules. Double sorted libraries were then grown out and DNA was collected for deep sequencing on a highseq. This DNA was also re-transformed onto plates and individual clones were picked for further analysis.

E. coli Toxin selection: Briefly, carbenicillin resistant plasmid containing an arabinose inducible toxin were transformed into E. coli cells and made electrocompetent. Biological triplicates of CasX DME Libraries with a toxin targeted guide RNA on a chloramphenicol resistant plasmid were transformed (at >5× library size) into said cells and grown in LB+CM and arabinose inducer. E. coli that cleaved the toxin plasmid survived in the induction media and were grown to mid log and plasmids with functional CasX cleavers were recovered. This selection was repeated as needed. Selected libraries were then grown out and DNA was collected for deep sequencing on a highseq. This DNA was also re-transformed onto plates and individual clones were picked for further analysis and testing.

Lentiviral based screen: Lentiviral particles were produced in HEK293 cells at a confluency of 70%-90% at time of transfection. Cells were transfected using polyethylenimine based transfection of plasmids containing a CasX DME library. Lentiviral vectors were co-transfected with the lentiviral packaging plasmid and the VSV-G envelope plasmids for particle production. Media was changed 12 hours post-transfection, and virus harvested at 36-48 hours post-transfection. Viral supernatants were filtered using 0.45 mm membrane filters, diluted in cell culture media if appropriate, and added to target cells HEK cells with an Integrated GFP reporter. Polybrene was supplemented to enhance transduction efficiency, if necessary. Transduced cells were selected for 24-48 hr post-transduction using puromycin and grown for 7-10 days. Cells were then sorted for GFP disruption & collected for highly functional CasX sgRNA or protein variants. Libraries were then Amplified via PCR directly from the genome and collected for deep sequencing on a highseq. This DNA could also be re-cloned and re-transformed onto plates and individual clones were picked for further analysis.

Assaying editing efficiency of an EGFP reporter: To assay the editing efficiency of CasX reference sgRNAs and proteins and variants thereof, EGFP HEK293T reporter cells were seeded into 96-well plates and transfected according to the manufacturer's protocol with lipofectamine 3000 (Life Technologies) and 100-200 ng plasmid DNA encoding a reference or variant CasX protein, P2A—puromycin fusion and the reference or variant sgRNA. The next day cells were selected with 1.5 μg/ml puromycin for 2 days and analyzed by fluorescence-activated cell sorting (FACS) 7 days after selection to allow for clearance of EGFP protein from the cells. EGFP disruption via editing was traced using an Attune NxT Flow Cytometer and high-throughput autosampler.

### Example 2: Cleavage Efficiency of CasX Reference sgRNA

The reference CasX sgRNA of SEQ ID NO: 4 (below) is described in WO 2018/064371, the contents of which are incorporated herein by reference.

(SEQ ID NO: 4)
ACAUCUGGCGCGUUUAUUCCAUUACUUUGGAGCCAGUCCCAGCGACUAU
GUCGUAUGGACGAAGCGCUUAUUUAUCGGAGAGAAACCGAUAAGUAAAA
CGCAUCAAAG.

It was found that alterations to the sgRNA reference sequence of SEQ ID NO: 4, producing SEQ ID NO: 5 (below) were able to improve CasX cleavage efficiency.

(SEQ ID NO: 5)
UACUGGCGCUUUUAUCUCAUUACUUUGAGAGCCAUCACCAGCGACUAUG
UCGUAUGGGUAAAGCGCUUAUUUAUCGGAGAGAAAUCCGAUAAAUAAGA
AGCAUCAAAG.

To assay the editing efficiency of CasX reference sgRNAs and variants thereof, EGFP HEK293T reporter cells were seeded into 96-well plates and transfected according to the manufacturer's protocol with lipofectamine 3000 (Life Technologies) and 100-200 ng plasmid DNA encoding a reference CasX protein, P2A—puromycin fusion and the sgRNA. The next day cells were selected with 1.5 μg/ml puromycin for 2 days and analyzed by fluorescence-activated cell sorting (FACS) 7 days after selection to allow for clearance of EGFP protein from the cells. EGFP disruption via editing was traced using an Attune NxT Flow Cytometer and high-throughput autosampler.

When testing cleavage of an EGFP reporter by CasX reference and sgRNA variants, the following spacer target sequences were used:

E6 (TGTGGTCGGGGTAGCGGCTG; SEQ ID NO: 29)
and
E7
(TCAAGTCCGCCATGCCCGAA; SEQ ID NO: 30).

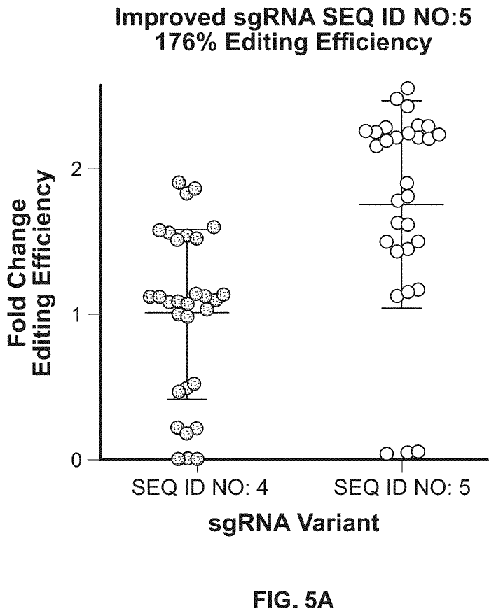

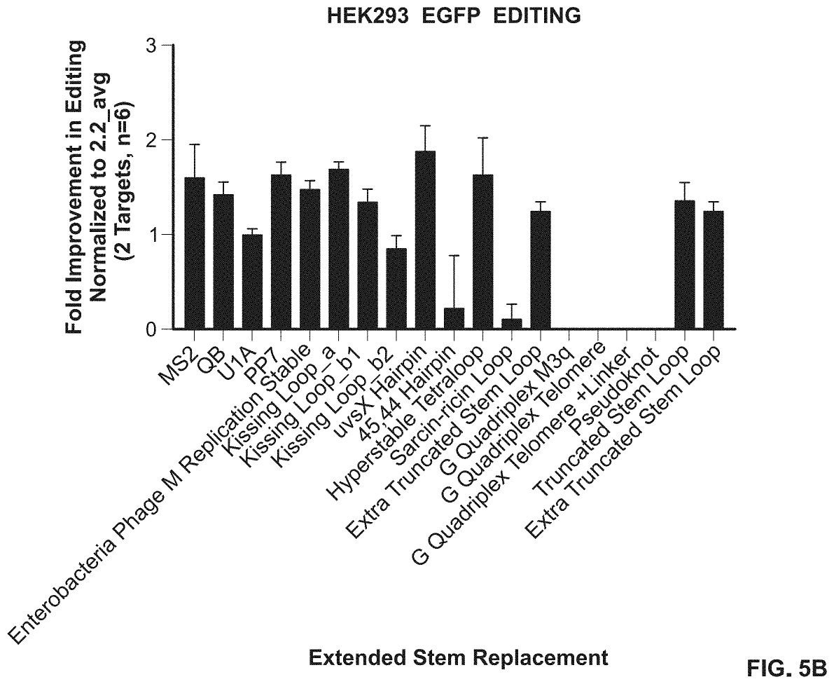

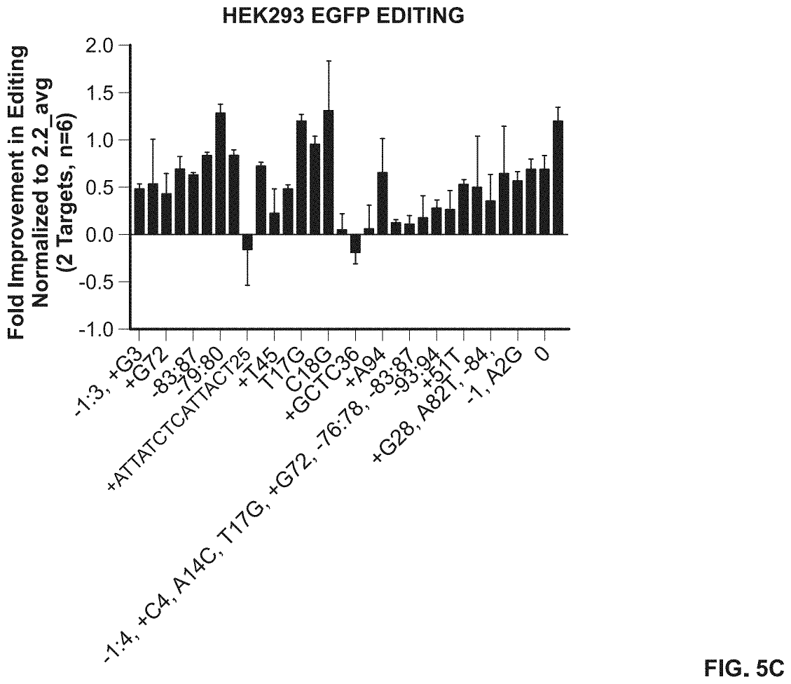

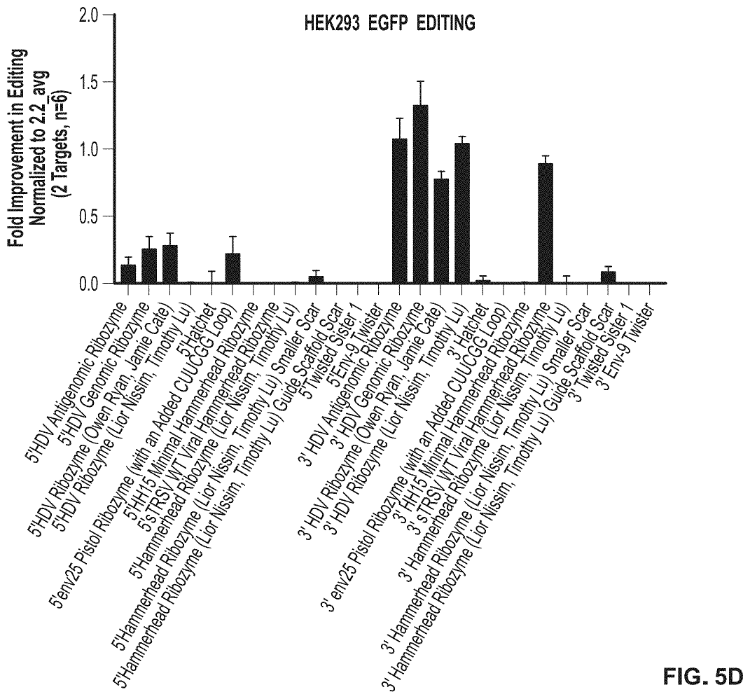

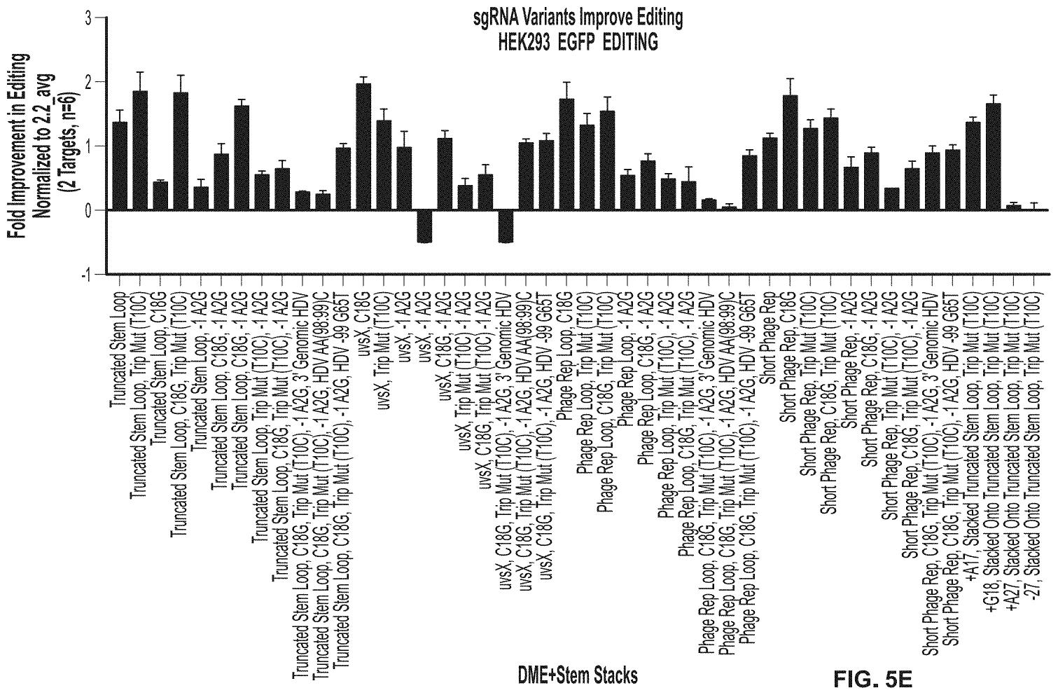

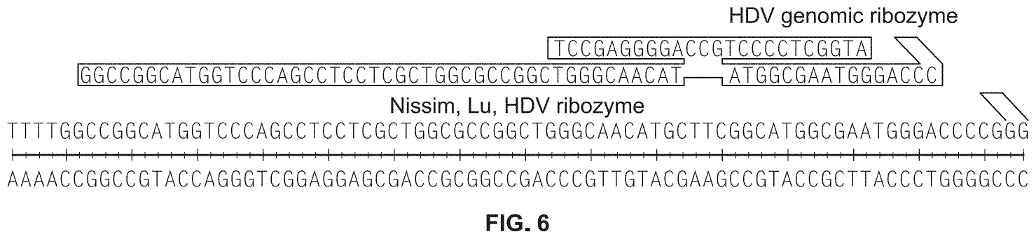

An example of the increased cleavage efficiency of the sgRNA of SEQ ID NO: 5 compared to the sgRNA of SEQ ID NO: 4 is shown in FIG. 5A. Editing efficiency of SEQ ID NO: 5 was improved 176% compared to SEQ ID NO: 4. Accordingly, SEQ ID NO: 5 was chosen as reference sgRNA for DME and additional sgRNA variant design, described below.

### Example 3: Mutagenesis of CasX References gRNA Produces Variants with Improved Target Cleavage

DME of the sgRNA was achieved using two distinct PCR methods. The first method, which generates single nucleotide substitutions, makes use of degenerate oligonucleotides. These are synthesized with a custom nucleotide mix, such that each locus of the primer that is complementary to the sgRNA locus has a 97% chance of being the wild type base, and a 1% chance of being each of the other three nucleotides. During PCR, the degenerate oligos anneal to, and just beyond, the sgRNA scaffold within a small plasmid, amplifying the entire plasmid. The PCR product was purified, ligated, and transformed into E. coli. The second method was used to generate sgRNA scaffolds with single or double nucleotide insertions and deletions. A unique PCR reaction was set up for each base pair intended for mutation: In the case of the CasX scaffold of SEQ ID NO: 5, 109 PCRs were used. These PCR primers were designed and paired such that PCR products were either missing a base pair, or contained an additional inserted base pair. For inserted base pairs, PCR primers inserted a degenerate base such that all four possible nucleotides were represented in the final library.

Once constructed, both the protein and sgRNA DME libraries were assayed in a screen or selection as described in Example 1 to quantitatively identify mutations conferring enhanced functionality. Any assay, such as cell survival or fluorescence intensity, is sufficient so long as the assay maintains a link between genotype and phenotype. High throughput sequencing of these populations and validating individual variant phenotypes provided information about mutations that affect functionality as assayed by screening or selection. Statistical analysis of deep sequencing data provided detailed insight into the mutation landscape and mechanism of protein function or guide RNA function (see FIGS. 3A-3B, FIG. 4A, 4B, 4C).

DME libraries of sgRNA variants were made using a reference gRNA of SEQ ID NO: 5, underwent selection or enrichment, and were sequenced to determine the fold enrichment of the sgRNA variants in the library. The libraries included every possible single mutation of every nucleotide, and double indels (insertion/deletions). The results are shown in FIGS. 3A-3B, FIGS. 4A-4C, and Tables 4-26 below.

To create a library of base pair substitutions using DME, two degenerate oligonucleotides that each bind to half of the sgRNA scaffold and together amplify the entire plasmid comprising the starting sgRNA scaffold were designed. These oligos were made from a custom nucleotide mix with a 3% mutation rate. These degenerate oligos were then used to PCR amplify the starting scaffold plasmid using standard manufacturing protocols. This PCR product was gel purified, again following standard protocols. The gel purified PCR product was then blunt end ligated and electroporated into an appropriate E. coli cloning strain. Transformants were grown overnight on standard media, and plasmid DNA was purified via miniprep.

To generate a library of small insertions and deletions, PCR primers were designed such that the PCR products resulting from amplification of the plasmid comprising the base sgRNA scaffold would either be missing a base pair, or contain an additional inserted base pair. For inserted base pairs, PCR primers were designed in which a degenerate base has been inserted, such that all four possible nucleotides were represented in the final library of pooled PCR products. The starting sgRNA scaffold was then PCR amplified with each set of oligos as their own reaction. Each PCR reaction contained five possible primers, although all primers annealed to the same sequence. For example, Primer 1 omitted a base, in order to create a deletion. Primers 2, 3, 4, and 5 inserted either an A, T, G, or C. However, these five primers all annealed to the same region and hence could be pooled in a single PCR. However, PCRs for different positions along the sgRNA needed to be kept in separate tubes, and 109 distinct PCR reactions were used to generate the sgRNA DME library.

The resulting 109 PCR products were then run on an agarose gel and excised before being combined and purified. The pooled PCR products were blunt ligated and electroporated into E. coli. Transformants were grown overnight on standard media with an appropriate selectable marker, and plasmid DNA was purified via miniprep. Having created a library of all single small indels, the steps of PCR amplifying the starting plasmid with each set of oligos, purifying, blunt end ligating, transforming into E. coli and miniprepping can be repeated to obtain a library containing most double small indels. Combining the single indel library and double indel library at a ratio of 1:1000 resulted in a library that represented both single and double indels.

The resulting libraries were then combined and passed through screening and/or selection process to identify variants with enhanced cleavage activity. DME libraries were screened using toxin cleavage and CRISPRi repression in E. coli, as well as EGFP cutting in lentiviral-transfected HEK293 cells, as described in Example 1. The fold enrichment of scaffold variants in DME libraries that have undergoing screening/selection followed by sequencing is shown below in Tables 4-26. The read counts associated with each of the below sequences in Tables 4-26 were determined (‘annotations’, ‘seq’). Only sequences with at least 10 reads across any sample were analyzed to filter from 15 Million to 600 K sequences. The below ‘seq’ gives the sequence of the entire insert between the two 5′ random 5mer and the 3′ random 5mer. ‘seq_short’ gives the anticipated sequence of the scaffold only. The mutations associated with each sequence were determined through alignment (‘muts’). All alterations are indicated by their [position (0-indexed)].[reference base].[alternate base]. Position 0 indicates the first T of the transcribed gRNA. Sequences with multiple mutations are semicolon separated. The column muts_1indexed, gives the same information but 1-indexed instead of 0-indexed. Each of the modifications are annotated (‘annotated_variants’), as being a single substitution/insertion/deletion, double substitution/insertion/deletion, single_del_single_sub (a deletion and an adjacent substitution), a single_sub_single_ins (a substitution and adjacent insertion), ‘outside_ref’ (indicates that the alteration is outside the transcribed gRNA), or ‘other’ (any larger substitution/insertion/deletion or some combination thereof). An insertion at position i indicates an inserted base between position i-1 and i (i.e. before the indicated position). To note about variant annotation: a deletion of any one of a consecutive set of bases can be attributed to any of those bases. Thus, a deletion of the T at position −1 is the same sequence as a deletion of the T at position 0. ‘counts’ indicates the sequencing-depth normalized read count per sequence per sample. Technical replicates were combined by taking the geometric mean. ‘log2enrichment’ gives the median enrichment (using a pseudocount of 10) across each context, or across all samples, after merging for technical replicates. The naive read count was averaged (geometric) between the D2_N and D3_N samples. Finally, the ‘log2enrichment_err’ gives the ‘confidence interval’ on the mean log2 enrichment. It is the standard deviation of the enrichment across samples*2/sqrt of the number of samples. Below, only the sequences with median log2enrichment−log2enrichment_err>0 are shown (2704/614564 sequences examined). Tables 4-26. Encoding sequences of exemplary CasX sg RNA variants and resulting activity. CI indicates confidence interval; MI indicates median enrichment, which indicates enhanced activity.

TABLE 4

| | SEQ | | | |
|---|---|---|---|---|
| index | ID NO | muts_1indexed | MI | 95% CI |
| 7240543 | 367 | 27.-.C; 76.G.- | 3.389759419 | 2.039653812 |
| 7240150 | 368 | 27.-.C; 75.-.0 | 3.111121121 | 1.861731632 |
| 2584994 | 369 | 0.T.-; 2.A.C; 27.-.0 | 2.99728039 | 1.806144082 |
| 2618163 | 370 | 0.T.-; 2.A.C; 55.-.G | 2.914525039 | 0.724917266 |
| 2655870 | 371 | 2.A.C; 0.T.-; 76.GG.-A | 2.902927654 | 0.391463755 |
| 2762330 | 372 | 2.A.C; 0.T.-; 55.-.T | 2.856516028 | 1.28972451 |
| 7247368 | 373 | 27.-.C; 86.C.- | 2.83486805 | 1.637226249 |
| 2731505 | 374 | 2.A.C; 0.T.-; 75.-.G | 2.79481581 | 0.624981577 |
| 2729600 | 375 | 2.A.C; 0.T.-; 76.-.T | 2.791450948 | 0.628411541 |
| 2701142 | 376 | 2.A.C; 0.T.-; 87.-.T | 2.767966305 | 0.559343857 |
| 2659588 | 377 | 2.A.C; 0.T.-; 75.-.0 | 2.732934068 | 0.47710005 |
| 2582823 | 378 | 0.T.-; 2.A.C; 27.-.A | 2.729090618 | 1.668805537 |
| 3000598 | 379 | 1.TA.--; 76.G.- | 2.704136598 | 0.439453245 |
| 10565036 | 380 | 15.-.T; 74.-.T | 2.681400766 | 0.808439581 |
| 9696472 | 381 | 28.-.T; 76.GG.-T | 2.681108849 | 1.714840304 |
| 2674674 | 382 | 2.A.C; 0.T.-; 86.-.0 | 2.6499525 | 0.771736317 |
| 7254130 | 383 | 27.-.C; 75.CG.-T | 2.62887552 | 1.755487816 |
| 2977442 | 384 | 1.TA.--; 55.-.G | 2.628550631 | 0.887370086 |
| 2661951 | 385 | 2.A.C; 0.T.-; 76.G.- | 2.626541337 | 0.431834643 |
| 1937646 | 386 | 2.A.C; 0.TT.--; 75.-.C | 2.626298021 | 1.328305588 |
| 2232796 | 387 | 0.T.-; 55.-.G | 2.606847968 | 0.776502589 |
| 2714418 | 388 | 0.T.-; 2.A.C; 81.GA.-T | 2.595247917 | 0.442508417 |
| 2700142 | 389 | 2.A.C; 0.T.-; 87.-.G | 2.581884688 | 0.608402275 |
| 2667512 | 390 | 2.A.C; 0.T.-; 77.GA.-- | 2.576796073 | 0.588238221 |
| 7239606 | 391 | 27.-.C; 76.-.A | 2.565846214 | 1.440612113 |
| 10563356 | 392 | 15.-.T; 75.-.G | 2.55742746 | 1.055615566 |
| 7181049 | 393 | 27.-.A; 75.-.0 | 2.542663573 | 1.893477285 |
| 2720034 | 394 | 2.A.C; 0.T.-; 78.-.0 | 2.5314705 | 0.491793711 |
| 2265581 | 395 | 0.T.-; 86.-.0 | 2.51980638 | 0.504274578 |
| 2256355 | 396 | 0.T.-; 76.GG.-C | 2.516497885 | 0.942311138 |
| 7251229 | 397 | 27.-.C; 76.-.G | 2.516430339 | 1.79266874 |
| 10281529 | 398 | 17.-.T; 76.GG.-A | 2.515423121 | 1.103585285 |
| 2299702 | 399 | 0.T.-; 74.-.T | 2.504423509 | 0.391893392 |
| 2670445 | 400 | 2.A.C; 0.T.-; 85.T.- | 2.498536138 | 1.225406412 |
| 2258816 | 401 | 0.T.-; 76.G.- | 2.494311051 | 0.474787855 |
| 7241311 | 402 | 27.-.C; 77.GA.-- | 2.492787478 | 1.594841999 |
| 2658150 | 403 | 2.A.C; 0.T.-; 76.GG.-C | 2.491526929 | 0.585113234 |
| 2734378 | 404 | 2.A.C; 0.T.-; 74.-.T | 2.489805276 | 0.484841997 |
| 2723181 | 405 | 2.A.C; 0.T.-; 76.-.6 | 2.488387029 | 0.421138525 |
| 2288202 | 406 | 0.T.-; 81.GA.-T | 2.487414543 | 0.591223915 |
| 2278172 | 407 | 0.T.-; 89.-.0 | 2.48621302 | 0.689529044 |
| 2997382 | 408 | 1.TA.--; 76.GG.-A | 2.465426966 | 1.066239003 |
| 255017 | 409 | 0.T.-:76.GG.-A | 2.463250003 | 0.421992457 |
| 2257399 | 410 | 0.T.-; 75.-.0 | 2.460412385 | 0.675576028 |
| 12183183 | 411 | 2.A.-; 81.GA.-T | 2.459190685 | 0.736058302 |
| 7252067 | 412 | 27.-.C; 76.GG.-T | 2.45896207 | 2.062274813 |
| 10525083 | 413 | 15.-.T; 75.-.0 | 2.448013673 | 1.006223409 |
| 7253869 | 414 | 27.-.C; 74.-.T | 2.439328513 | 1.638183736 |
| 4303777 | 415 | 4.T.-; 76.-.T | 2.435110112 | 0.781688536 |
| 2741395 | 416 | 2.A.C; 0.T.-; 73.A.- | 2.434901914 | 0.633362915 |
| 7250940 | 417 | 27.-.C; 78.A.- | 2.423359724 | 2.064125021 |
| 4302595 | 418 | 4.T.-; 76.GG.-T | 2.42205606 | 0.850176631 |
| 4275786 | 419 | 4.T.-; 87.-.T | 2.419947604 | 1.019110537 |
| 2650980 | 420 | 2.A.C; 0.T.-; 74.-.0 | 2.414107731 | 0.461696916 |
| 2458336 | 421 | 1.TA.--; 3.C.A; 76.G.- | 2.410845711 | 1.088632737 |
| 10284144 | 422 | 17.-.T; 76.G.- | 2.406246674 | 1.637908059 |
| 2726809 | 423 | 2.A.C; 0.T.-; 76.G.-; | 2.400026208 | 0.556489787 |
| | | 78.A.T | | |
| 2280896 | 424 | 0.T.-; 87.-.T | 2.398060925 | 0.559723653 |
| 2673790 | 425 | 2.A.C; 0.T.-; 88.G.- | 2.39801837 | 1.017283194 |
| 3188700 | 426 | 0.T.-; 2.A.G; 27.-.0 | 2.394340831 | 1.73237167 |
| 9632434 | 427 | 16.------------. | 2.393572747 | 1.140837334 |
| | | CTCATTACTTTG; | | |
| | | 75.-.G | | |
| 3029757 | 428 | 1.TA.--; 78.A.- | 2.391614326 | 0.52432112 |
| 2728393 | 429 | 2.A.C; 0.T.-; 76.GG.-T | 2.390176219 | 0.714223997 |
| 2300381 | 430 | 0.T.-; 75.CG.-T | 2.385232105 | 0.948093789 |
| 2279969 | 431 | 0.T.-; 86.C.- | 2.382152098 | 0.403913543 |
| 2260011 | 432 | 0.T.-; 77.-.0 | 2.379187705 | 0.60793876 |
| 2248579 | 433 | 0.T.-; 72.-.0 | 2.377033686 | 0.742558535 |
| 12075394 | 434 | 2.A.-; 55.-.G | 2.376878541 | 0.679081085 |
| 9602743 | 435 | 28.-.C; 76.GG.-C | 2.376348735 | 1.680837509 |
| 2736722 | 436 | 2.A.C; 0.T.-; 73.AT.-C | 2.374354239 | 1.104279695 |
| 12117240 | 437 | 2.A.-; 76.GG.-A | 2.372161723 | 0.428593735 |
| 10307397 | 438 | 17.-.T; 78.-.0 | 2.365042525 | 0.867959934 |
| 3034775 | 439 | 1.TA.--; 75.-.G | 2.359826914 | 0.99152259 |
| 12030812 | 440 | 2.A.-; 27.-.A | 2.355284207 | 1.651243725 |
| 10530683 | 441 | 15.-.T; 86.-.A | 2.354920575 | 0.999356279 |
| 12202799 | 442 | 2.A.-; 75.-.G | 2.352119205 | 0.508202346 |
| 9687168 | 443 | 28.-.T; 76.GG.-A | 2.350792044 | 1.612399102 |
| 4309853 | 444 | 4.T.-; 75.CG.-T | 2.344380848 | 0.844586894 |
| 4234320 | 445 | 4.T.-; 75.-.0 | 2.343966564 | 0.820229568 |
| 2698521 | 446 | 2.A.C; 0.T.-; 88.-.T | 2.33926209 | 0.684535077 |
| 2253698 | 447 | 0.T.-; 75.-.A | 2.33353651 | 0.918413016 |
| 2468003 | 448 | 1.TA.--; 3.C.A; 75.-.G | 2.329652898 | 0.934127399 |
| 12290253 | 449 | 2.A.-; 28.-.0 | 2.326187914 | 1.587751482 |
| 2999382 | 450 | 1.TA.--; 75.-.0 | 2.315411787 | 0.591810721 |
| 3227871 | 451 | 2.A.G; 0.T.-; 55.-.G | 2.313991155 | 0.774330181 |
| 10521017 | 452 | 15.-.T; 74.-.0 | 2.313768991 | 0.910046563 |
| 10089663 | 453 | 19.-.T; 75.-.G | 2.308273929 | 1.077849871 |
| 4274894 | 454 | 4.T.-; 87.-.G | 2.308046437 | 0.511567574 |
| 2466567 | 455 | 1.TA.--; 3.C.A; 78.A.- | 2.307828141 | 1.291273333 |
| 2696261 | 456 | 2.A.C; 0.T.-; 89.-.0 | 2.292578418 | 0.680820688 |
| 2675948 | 457 | 2.A.C; 0.T.-; 89.-.A | 2.289131671 | 1.259062601 |
| 10521784 | 458 | 15.-.T; 74.-.G | 2.282950048 | 0.904736128 |
| 12123787 | 459 | 2.A.-; 76.G.- | 2.27754961 | 0.49194122 |
| 10310335 | 460 | 17.-.T; 76.GG.-T | 2.27478155 | 0.80367504 |
| 2295876 | 461 | 0.T.-; 77.-.T | 2.273004186 | 0.931439741 |
| 2697871 | 462 | 0.T.-; 2.A.C; 89.-.T | 2.250463711 | 0.626247893 |
| 2735417 | 463 | 2.A.C; 0.T.-; 75.CG.-T | 2.249451799 | 0.389761214 |
| 2671836 | 464 | 0.T.-; 2.A.C; 86.-.A | 2.245473306 | 0.542416673 |
| 12033345 | 465 | 2.A.-; 27.-.C | 2.235034582 | 1.903166042 |

TABLE 5

| | SEQ | | | |
|---|---|---|---|---|
| | ID | | | |
| index | NO | muts_1indexed | MI | 95% CI |
| 2821484 | 466 | 0.T.-; 2.A.C; 17.-T. | 2.234604485 | 0.750279684 |
| 3033813 | 467 | 1.TA.--; 76.-.T | 2.229483844 | 0.547530348 |
| 2291551 | 468 | 0.T.-; 78.-.0 | 2.226391312 | 0.53155696 |
| 2716457 | 469 | 2.A.C; 0.T.-; 80.A.- | 2.212685904 | 0.548257242 |
| 2697599 | 470 | 2.A.C; 0.T.-; 89.A.- | 2.209480847 | 1.345862006 |
| 12135440 | 471 | 2.A.-; 87.-.A | 2.208341827 | 1.052844724 |
| 4273350 | 472 | 4.T.-; 88.-.T | 2.207860033 | 1.012912804 |
| 2298121 | 473 | 0.T.-; 75.-.G | 2.207579751 | 0.240933007 |
| 2652510 | 474 | 0.T.-; 2.A.C; 74.-.G | 2.206487468 | 0.612576212 |
| 3006640 | 475 | 1.TA.--; 86.-.0 | 2.206221139 | 0.584000131 |
| 10313388 | 476 | 17.-.T; 74.-.T | 2.206178293 | 1.036335839 |
| 10081410 | 477 | 19.-.T; 87.-.G | 2.205894948 | 0.589463833 |
| 3033236 | 478 | 1.TA.--; 76.GG.-T | 2.198134613 | 0.669434462 |
| 7242523 | 479 | 27.-.C86.-.0 | 2.198004115 | 1.972713412 |
| 7254383 | 480 | 27.-.C; 73.AT.-C | 2.19783418 | 1.510443212 |
| 2264531 | 481 | 0.T.-; 87.-.A | 2.197793214 | 0.777981784 |
| 2727301 | 482 | 0.T.-; 2.A.C; 77.-.T | 2.196877578 | 1.323161971 |
| 3019306 | 483 | 1.TA.--; 87.-.G | 2.191451738 | 0.53442114 |
| 4295725 | 484 | 4.T.-; 78.A.- | 2.187137221 | 0.609047392 |
| 10311816 | 485 | 17.-.T75.-.G | 2.187062055 | 1.506790657 |
| 12167745 | 486 | 2.A.-; 87.-.G | 2.184448369 | 0.736092188 |
| 12199256 | 487 | 2.A.-; 76.GG.-T | 2.178714409 | 0.736646546 |
| 6477911 | 488 | 16.-.C; 75.-.G | 2.177618084 | 0.983309644 |
| 4274124 | 489 | 4.T.-; 86.C.- | 2.17055291 | 0.474178023 |
| 12206105 | 490 | 2.A.-; 74.-.T | 2.170189846 | 0.60843597 |
| 12166825 | 491 | 2.A.-; 86.C.- | 2.167668003 | 0.773946533 |
| 11956698 | 492 | 2.AC.--; 43.C; 86.-.0 | 2.164335553 | 1.359888436 |
| 2280390 | 493 | 0.T.-; 87.-.G | 2.162228704 | 0.478769807 |
| 2650159 | 494 | 2.A.C; 0.T.-; 74.T. | 2.160583429 | 0.51707006 |
| 10531253 | 495 | 15.-.T; 87.-.A | 2.15924529 | 1.129639708 |
| 2665054 | 496 | 2.A.C; 0.T.-; 79.G.- | 2.157940781 | 0.562020183 |
| 8531520 | 497 | 75.-.G; 86.-.0 | 2.154823863 | 0.581992186 |
| 2296436 | 498 | 0.T.-; 76.GG.-T | 2.153923256 | 0.67936875 |
| 4249048 | 499 | 4.T.-; 86.-.0 | 2.142285584 | 0.675472603 |
| 10547068 | 500 | 15.-.T; 87.-.G | 2.139808506 | 0.856696675 |
| 12168820 | 501 | 2.A.-; 87.-.T | 2.139576287 | 0.458066181 |
| 2466824 | 502 | 1.TA.--; 3.C.A; 76.-.6 | 2.137393958 | 0.98855471 |
| 3036963 | 503 | 1.TA.--; 75.CG.-T | 2.136816031 | 0.479393618 |
| 10522450 | 504 | 15.-.T; 75.-.A | 2.134930675 | 1.003462809 |
| 10300736 | 505 | 17.-.T87.-.T | 2.134132228 | 1.348111441 |
| 3002220 | 506 | 1.TA.--; 79.G.- | 2.131038893 | 0.607179239 |
| 3030471 | 507 | 1.TA.--; 76.-.G | 2.129810368 | 0.371633581 |
| 10523429 | 508 | 15.-.T; 76.GG.-A | 2.129808628 | 0.787404871 |
| 1909254 | 509 | 0.TTA.---; 3.C.A; 75.-.G | 2.129733196 | 1.147227186 |
| 3004722 | 510 | 1.TA.--; 85.T.- | 2.123755125 | 1.091994071 |
| 2672731 | 511 | 2.A.C; 0.T.-; 87.-.A | 2.121163195 | 0.897965834 |
| 12129733 | 512 | 2.A.-; 77.GA.-- | 2.11956301 | 0.499892769 |
| 4250089 | 513 | 4.T.-; 89.-.A | 2.116592595 | 0.997715957 |
| 2688981 | 514 | 2.A.C; 0.T.-; 99.-.G | 2.112345173 | 0.980184341 |
| 2995452 | 515 | 1.TA.--; 74.-.G | 2.112014409 | 0.610553646 |
| 12114782 | 516 | 2.A.-; 75.-.A | 2.110203616 | 0.499880843 |
| 2993173 | 517 | 1.TA.--; 73.-.A | 2.10375793 | 0.696850789 |
| 1978344 | 518 | 0.T.C; 87.-.G | 2.100156515 | 0.870067465 |
| 4294004 | 519 | 4.T.-; 78.-.0 | 2.098823408 | 0.595418093 |
| 10568306 | 520 | 15.-.T; 73.A.- | 2.096194341 | 0.741080975 |
| 10561545 | 521 | 15.-.T; 76.GG.-T | 2.095379508 | 0.553757689 |
| 2713433 | 522 | 2.A.C; 0.T.-; 82.AA.-T | 2.094347694 | 0.559870514 |
| 1863579 | 523 | 0.TT.--; 75.-.G | 2.086195215 | 0.787239435 |
| 3006303 | 524 | 1.TA.--; 88.G.- | 2.086194701 | 0.536507797 |
| 4236935 | 525 | 4.T.-; 76.G.- | 2.081251549 | 0.919447585 |
| 12138801 | 526 | 2.A.-; 89.-.A | 2.079884636 | 1.115488685 |
| 12164760 | 527 | 2.A.-; 89.-.T | 2.079725529 | 0.315885203 |
| 10288787 | 528 | 17.-.T; 86.-.0 | 2.079540543 | 0.927030301 |
| 2664128 | 529 | 0.T.-2.A.C; 77.-.C | 2.079234701 | 0.378694546 |
| 2663861 | 530 | 0.T.-; 2.A.C; 76.G.-; | 2.077930225 | 0.700390601 |
| | | 78.A.C | | |
| 2726063 | 531 | 0.T.-; 2.A.C; 78.A.T | 2.077653454 | 0.972036971 |
| 4232837 | 532 | 4.T.-; 76.GG.-C | 2.068589675 | 0.579547915 |
| 3001194 | 533 | 1.TA.--; 77.-.A | 2.062571166 | 0.628957326 |
| 2048069 | 534 | 0.TT.--; 2.A.G; 76.G.- | 2.05862732 | 1.413051852 |
| 2653681 | 535 | 2.A.C; 0.T.-; 75.-.A | 2.051977832 | 0.427290312 |
| 2265126 | 536 | 0.T.-; 88.G.- | 2.050226061 | 0.556563218 |
| 2739399 | 537 | 0.T.-; 2.A.C; 73.A.G | 2.049449237 | 1.003306718 |
| 7250543 | 538 | 27.-.C; 78.-.C | 2.047334217 | 1.480241124 |
| 2747651 | 539 | 0.T.-; 2.A.C66.0 | 2.046981233 | 0.899726699 |
| 12437734 | 540 | 1.TAC.---; 78.A.- | 2.043018072 | 0.614544855 |
| 2826230 | 541 | 0.T.-; 2.A.C; 15.-.T | 2.041901776 | 0.537816622 |
| 2709008 | 542 | 2.A.C; 0.T.-; 82.A.-; | 2.036707329 | 1.246046649 |
| | | 84.A.T | | |
| 3005336 | 543 | 1.TA.--; 86.-.A | 2.034175728 | 0.483054171 |
| 4301274 | 544 | 4.T.-; 76.G.-; 78.A.T | 2.028068229 | 0.873353997 |
| 3018865 | 545 | 1.TA.--; 86.C.- | 2.024668973 | 0.616204139 |
| 2699310 | 546 | 2.A.C; 0.T.-; 86.0.- | 2.023086951 | 0.563791987 |
| 2279026 | 547 | 0.T.-; 89.A.- | 2.022323648 | 1.568173921 |
| 7248209 | 548 | 27.-.C; 82.A.- | 2.022242177 | 1.626724535 |
| 10562113 | 549 | 15.-.T; 76.-.T | 2.019995187 | 0.857776668 |
| 7181373 | 550 | 27.-.A; 76.G.- | 2.014441438 | 1.907810918 |
| 10559019 | 551 | 15.-.T; 76.-.G | 2.014069707 | 0.752817112 |
| 3018452 | 552 | 1.TA.--; 88.-.T | 2.012932283 | 0.626313379 |

TABLE 6

| | SEQ | | | |
|---|---|---|---|---|
| | ID | | | |
| index | NO | muts_1indexed | MI | 95% CI |
| 12118457 | 553 | 2.A.-; 76.-.A | 2.011043775 | 1.170428809 |
| 2805043 | 554 | 2.A.C; 0.T.-; 28.-.0 | 2.009926076 | 1.5236908 |
| 4242379 | 555 | 4.T.-; 77.GA.-- | 2.007947564 | 0.98469627 |
| 2259846 | 556 | 0.T.-; 76.6.-; 78.A.0 | 2.004816439 | 0.640251884 |
| 6462092 | 557 | 16.-.C; 87.-.A | 2.001230775 | 0.982714839 |
| 4312495 | 558 | 4.T.-; 73.AT.-G | 1.997381596 | 0.707994266 |
| 2668714 | 559 | 0.T.-; 2.A.C; 81.GA.-C | 1.996012534 | 0.678455572 |
| 2294477 | 560 | 0.T.-; 78.AG.-T | 1.993651117 | 0.703085174 |
| 12198135 | 561 | 2.A.-; 77.-.T | 1.993577573 | 1.432706828 |
| 4238150 | 562 | 4.T.-; 77.-.A | 1.992607238 | 0.761786326 |
| 3019738 | 563 | 1.TA.--; 87.-.T | 1.992446303 | 0.532459966 |
| 2352050 | 564 | 0.T.-; 17.-.T | 1.991048683 | 0.852386811 |
| 2705912 | 565 | 2.A.C; 0.T.-; 83.-.0 | 1.99036719 | 0.585299092 |
| 6478822 | 566 | 16.-.C; 74.-.T | 1.988911775 | 0.477065619 |
| 2665913 | 567 | 2.A.C; 0.T.-; 79.GA.-C | 1.9871574 | 1.186495063 |
| 3331447 | 568 | 2.A.G; 0.T.-; 76.GG.-T | 1.984971034 | 0.958178637 |
| 3186538 | 569 | 2.A.G; 0.T.-; 27.-.A | 1.983054551 | 1.530372349 |
| 2738784 | 570 | 2.A.C; 0.T.-; 73.AT.-G | 1.977333796 | 0.62344263 |
| 7832272 | 571 | 55.-.G | 1.976646956 | 0.881875422 |
| 4297458 | 572 | 4.T.-; 76.-.G | 1.976295522 | 0.996798704 |
| 3334291 | 573 | 2.A.G; 0.T.-; 75.-.G | 1.975325989 | 0.653653125 |
| 2212416 | 574 | 0.T.-; 27.-.0 | 1.973859043 | 1.457984475 |
| 8752897 | 575 | 55.-.T; 76.G.- | 1.971785265 | 0.46834501 |
| 2293333 | 576 | 0.T.-36.-.G | 1.970005749 | 0.514281315 |
| 7180386 | 577 | 27.-.A; 76.GG.-A | 1.969392489 | 1.667131306 |
| 2996180 | 578 | 1.TA.--; 75.-.A | 1.966703028 | 0.475623563 |
| 7238423 | 579 | 27.-.C; 74.T.- | 1.962642235 | 1.563372071 |
| 2261752 | 580 | 0.T.-; 77.GA.-- | 1.961634278 | 0.503084863 |
| 10282247 | 581 | 17.-.T; 76.GG.-C | 1.960039354 | 0.718769466 |
| 4230973 | 582 | 4.T.-; 76.GG.-A | 1.958471711 | 0.723493647 |
| 4276520 | 583 | 4.T.-; 86.-.G | 1.958025163 | 0.900653677 |
| 2675193 | 584 | 0.T.-; 2.A.C; 88.GA.-C | 1.956983044 | 0.878446278 |
| 13101476 | 585 | -1.GT.--; 75.-.G | 1.952447041 | 0.438583434 |
| 7203209 | 586 | 27.G.-76.GG.-C | 1.952129576 | 1.708559549 |
| 2724398 | 587 | 0.T.-; 2.A.C; 78.A.G | 1.947253829 | 0.801326607 |
| 10309365 | 588 | 17.-.T; 78.-.T | 1.946957778 | 1.542210263 |
| 10520418 | 589 | 15.-.T; 74.T.- | 1.944704908 | 0.727975608 |
| 10300394 | 590 | 17.-.T; 87.-.0 | 1.943744986 | 1.037237205 |
| 4248302 | 591 | 4.T.-; 88.G.- | 1.936753816 | 0.857321817 |
| 7240856 | 592 | 27.-.C; 76.G.-; 78.A.0 | 1.936751382 | 1.187952295 |
| 4313003 | 593 | 4.T.-; 73.A.G | 1.935442861 | 0.687757679 |
| 2467599 | 594 | 1.TA.--; 3.C.A; 76.GG.-T | 1.92287425 | 1.104512209 |
| 2279202 | 595 | 0.T.-; 89.-.T | 1.921076549 | 0.70944656 |
| 2259410 | 596 | 0.T.-; 77.-.A | 1.920454929 | 0.417160464 |
| 4305674 | 597 | 4.T.-; 75.-.G | 1.915266489 | 1.088551012 |
| 6459602 | 598 | 16.-.C; 76.G.- | 1.914798378 | 0.642358195 |
| 2701869 | 599 | 0.T.-; 2.A.C; 86.-.G | 1.914049421 | 0.477347775 |
| 2252978 | 600 | 0.T.-; 74.-.G | 1.911378422 | 0.602397906 |
| 6470049 | 601 | 16.-.C; 87.-.G | 1.910419486 | 0.714796483 |
| 12134362 | 602 | 2.A.-; 86.-.A | 1.906851105 | 0.661062722 |
| 12209524 | 603 | 2.A.-; 73.A.0 | 1.901209161 | 1.154288772 |
| 2260529 | 604 | 0.T.-; 79.G.- | 1.899530324 | 0.82876912 |
| 2690549 | 605 | 0.T.-; 2.A.C; 98.-.T | 1.898891625 | 0.95407757 |
| 10073100 | 606 | 19.-.T; 88.G.- | 1.89794244 | 0.781693777 |
| 4239969 | 607 | 4.T.-; 79.G.- | 1.897769811 | 0.794035202 |
| 3026047 | 608 | 1.TA.--; 81.GA.-T | 1.896236907 | 0.554505707 |
| 3003294 | 609 | 1.TA.--; 77.GA.-- | 1.895773589 | 0.506363603 |
| 12121216 | 610 | 2.A.-; 75.-.0 | 1.895093657 | 0.610069511 |
| 2696635 | 611 | 0.T.-; 2.A.C; 89.AT.-G | 1.893880561 | 0.881556619 |
| 12130978 | 612 | 2.A.-; 81.GA.-C | 1.891473979 | 0.935650632 |
| 6475473 | 613 | 16.-.C; 78.A.- | 1.888788297 | 0.580982578 |
| 1853356 | 614 | 0.TT.--; 76.G.- | 1.884632638 | 0.80171104 |
| 8544082 | 615 | 75.-.G; 87.-.G | 1.884341912 | 0.535653292 |
| 2884429 | 616 | 1.-.C; 76.6.- | 1.883538595 | 0.673377662 |
| 6368955 | 617 | 17.-.A; 76.-.G | 1.882010313 | 0.843102729 |
| 2746170 | 618 | 2.A.C; 0.T.-; 66.CT.-G | 1.87989538 | 0.516685509 |
| 4226314 | 619 | 4.T.-; 74.-.0 | 1.873701307 | 0.901044909 |
| 6304607 | 620 | 16.-.A; 76.G.- | 1.873365067 | 0.522811196 |
| 2583788 | 621 | 0.T.-; 2.A.C; 27.G.- | 1.873101254 | 1.38825951 |
| 2255694 | 622 | 0.T.-; 76.-.A | 1.869207789 | 0.836610884 |
| 7249882 | 623 | 27.-.C; 80.A.- | 1.867026014 | 1.645069173 |
| 10069481 | 624 | 19.-.T; 75.-.0 | 1.864128274 | 0.644689284 |
| 2643173 | 625 | 0.T.-; 2.A.C; 70.T.- | 1.863776691 | 1.688937677 |
| 12749699 | 626 | 0.-.T; 75.-.G | 1.863460232 | 0.756791498 |
| 7208859 | 627 | 27.G.-; 87.-.G | 1.861951751 | 1.68656168 |
| 4271233 | 628 | 4.T.-; 89.-.0 | 1.854344144 | 0.839274714 |
| 6455215 | 629 | 16.-.C; 73.-.A | 1.850284678 | 0.825458676 |
| 2816525 | 630 | 0.T.-; 2.A.C; 19.-.T | 1.847987652 | 0.368770724 |
| 2292594 | 631 | 0.T.-; 78.A.- | 1.846146605 | 0.312862911 |
| 2287708 | 632 | 0.T.-; 82.AA.-T | 1.845505779 | 0.408363625 |
| 2721779 | 633 | 2.A.C; 0.T.-; 78.A.- | 1.842043235 | 0.676554896 |
| 1945942 | 634 | 0.TT.--; 2.A.C; 75.-.G | 1.841650114 | 1.270815664 |
| 12111705 | 635 | 2.A.-; 74.-.0 | 1.840532416 | 0.668977898 |

TABLE 7

| | SEQ | | | |
|---|---|---|---|---|
| index | ID NO | muts_1indexed | MI | 95% CI |
| 2567750 | 636 | 0.T.-; 2.A.C; 16.-.0 | 1.8403251 | 0.426712425 |
| 2463364 | 637 | 1.TA.--; 3.C.A; 87.-.G | 1.839213942 | 0.821355081 |
| 3031594 | 638 | 1.TA.--; 78.AG.-T | 1.838954225 | 0.619562955 |
| 10199376 | 639 | 18.-.G; 75.-.G | 1.837121283 | 1.238162985 |
| 4272444 | 640 | 4.T.-; 89.A.- | 1.836884745 | 0.9982317 |
| 9610551 | 641 | 28.-.C; 78.A.- | 1.835988851 | 1.801689999 |
| 2737747 | 642 | 0.T.-; 2.A.C; 73.A.0 | 1.832606597 | 1.293143415 |
| 12113430 | 643 | 2.A.-; 74.-.G | 1.828115917 | 0.752764013 |
| 10530413 | 644 | 15.-.T; 85.TC.-G | 1.825064554 | 1.155205145 |
| 12176759 | 645 | 2.A.-; 83.-.T | 1.824304802 | 1.045532305 |
| 12127185 | 646 | 2.A.-79.0.- | 1.824126309 | 0.605894284 |
| 4288099 | 647 | 4.T.-; 81.GA.-T | 1.823734764 | 0.75329209 |
| 12196850 | 648 | 2.A.-; 78.A.T | 1.82118191 | 1.085783969 |
| 6457366 | 649 | 16.-.C; 75.-.A | 1.820899999 | 0.638027421 |
| 12105140 | 650 | 2.A.-; 72.-.0 | 1.818449485 | 0.69990752 |
| 1944577 | 651 | 0.TT.--; 2.A.C; 78.A.- | 1.816800398 | 1.169943299 |
| 4293546 | 652 | 4.T.-; 78.AG.-C | 1.815616502 | 1.015355487 |
| 9996838 | 653 | 19.-.G; 74.-.T | 1.814174099 | 0.799877397 |
| 10301024 | 654 | 17.-.T; 86.-.G | 1.813594662 | 0.966656071 |
| 2308228 | 655 | 0.T.-; 66.C.- | 1.811408251 | 0.755819624 |
| 7835938 | 656 | 55.-.G; 75.-.G | 1.811344956 | 1.11212595 |
| 3005841 | 657 | 1.TA.--; 87.-.A | 1.810592015 | 0.805934793 |
| 12169698 | 658 | 2.A.-; 86.-.G | 1.807867405 | 0.857412996 |
| 3028597 | 659 | 1.TA.--; 78.AG.-C | 1.802701874 | 0.743214495 |
| 7191855 | 660 | 27.-.A; 75.CG.-T | 1.802109849 | 1.429792639 |
| 9972503 | 661 | 19.-.G; 74.T.- | 1.801952299 | 0.749871626 |
| 4026979 | 662 | 3.-.C; 75.-.G | 1.801908368 | 1.374192028 |
| 7180118 | 663 | 27.-.A; 75.-.A | 1.801182739 | 1.524863174 |
| 10081203 | 664 | 19.-.T; 86.C.- | 1.799229513 | 0.502156779 |
| 10532156 | 665 | 15.-.T; 86.-.0 | 1.796941605 | 1.070232668 |
| 2749667 | 666 | 2.A.C; 0.T.-; 65.GC.-T | 1.795230574 | 0.641741966 |
| 12139228 | 667 | 2.A.-; 90.-.0 | 1.793917598 | 1.201242724 |
| 10288547 | 668 | 17.-.T; 88.G.- | 1.793873519 | 1.192733019 |
| 4331367 | 669 | 4.T.-; 55.-.T | 1.792669241 | 0.481210459 |
| 2725463 | 670 | 2.A.C; 0.T.-; 78.-.T | 1.79217915 | 0.507302457 |
| 2718857 | 671 | 0.T.-; 2.A.C; 79.GA.-T | 1.791913163 | 0.899839665 |
| 2247247 | 672 | 0.T.-; 72.-.A | 1.791822909 | 0.887353696 |
| 12125011 | 673 | 2.A.-; 77.-.A | 1.786430219 | 0.527171387 |
| 4225246 | 674 | 4.T.-; 74.T.- | 1.786417427 | 0.629044775 |
| 12165722 | 675 | 2.A.-; 88.-.T | 1.786308399 | 1.272797742 |
| 2733129 | 676 | 0.T.-; 2.A.C; 75.C.- | 1.785722582 | 0.560847969 |
| 2469676 | 677 | 1.TA.--; 3.C.A; 73.A.- | 1.785269687 | 1.17402736 |
| 3018172 | 678 | 1.TA.--; 89.-.T | 1.784650459 | 0.75738752 |
| 12196049 | 679 | 2.A.-; 78.-.T | 1.782353237 | 0.753905536 |
| 9612063 | 680 | 28.-.C; 74.-.T | 1.782091765 | 1.617793957 |
| 10547909 | 681 | 15.-.T86.-.G | 1.781475153 | 0.81786269 |
| 12194342 | 682 | 2.A.-; 78.A.-; 80.A.- | 1.77971829 | 1.288558347 |
| 4228855 | 683 | 4.T.-; 75.-.A | 1.775913052 | 0.896674597 |
| 10546613 | 684 | 15.-.T; 86.C.- | 1.775790253 | 0.858668751 |
| 10547538 | 685 | 15.-.T; 87.-.T | 1.771955914 | 1.080256702 |
| 10519772 | 686 | 15.-.T; 73.-.A | 1.770892898 | 0.624353321 |
| 8510297 | 687 | 77.G.T | 1.76973633 | 1.238813589 |
| 12119606 | 688 | 2.A.-; 76.GG.-C | 1.768206821 | 1.109938596 |
| 2669299 | 689 | 0.T.-; 2.A.C; 85.TC.-A | 1.766862971 | 0.841676179 |
| 6469807 | 690 | 16.-.C; 86.C.- | 1.764660394 | 0.758824717 |
| 10197299 | 691 | 18.-.G; 76.-.G | 1.763760462 | 0.832130059 |
| 3344225 | 692 | 2.A.G; 0.T.-; 73.A.- | 1.76219764 | 1.216224489 |
| 2456917 | 693 | 1.TA.--; 3.C.A; 75.-.A | 1.760739771 | 1.203417145 |
| 10307233 | 694 | 17.-.T; 78.AG.-C | 1.760381908 | 1.100594294 |
| 12314352 | 695 | 2.A.-; 15.-.T | 1.758187872 | 0.435582357 |
| 12177388 | 696 | 2.A.-; 82.AA.-- | 1.750995276 | 0.61463172 |
| 2694455 | 697 | 0.T.-; 2.A.C; 91.A.-; | 1.750810727 | 1.014669774 |
| | | 93.A.G | | |
| 3040066 | 698 | 1.TA.--; 73.A.- | 1.750348973 | 0.689636186 |
| 10081633 | 699 | 19.-.T87.-.T | 1.749883408 | 0.917269067 |
| 4246508 | 700 | 4.T.-; 86.-.A | 1.748983402 | 0.938986874 |
| 4301580 | 701 | 4.T.-; 77.-.T | 1.743946631 | 0.701295877 |
| 10181172 | 702 | 18.-.G; 75.-.A | 1.743101698 | 1.01566765 |
| 12200668 | 703 | 2.A.-; 76.-.T | 1.740748942 | 0.87292689 |
| 10524336 | 704 | 15.-.T; 76.GG.-C | 1.738223203 | 0.390480555 |
| 3007212 | 705 | 1.TA.--; 89.-.A | 1.737858461 | 1.071814108 |
| 10526271 | 706 | 15.-.T; 76.G.- | 1.737620179 | 1.09826626 |
| 10561166 | 707 | 15.-.T; 77.-.T | 1.736588831 | 0.744748617 |
| 2663037 | 708 | 2.A.C; 0.T.-; 77.-.A | 1.731783986 | 0.417310116 |
| 12136525 | 709 | 2.A.-; 88.G.- | 1.731312294 | 0.57794653 |
| 8758832 | 710 | 55.-.T; 78.A.- | 1.730884483 | 0.640655822 |
| 1864295 | 711 | 0.TT.--; 75.CG.-T | 1.7286748 | 0.424298588 |
| 10550736 | 712 | 15.-.T; 82.A.-; 84.A.G | 1.728100107 | 0.887580069 |
| 2657071 | 713 | 2.A.C; 0.T.-; 76.-.A | 1.727660257 | 1.206003654 |
| 2059338 | 714 | 0.TT.--; 2.A.G; 75.-.G | 1.725033887 | 1.054075378 |
| 12182224 | 715 | 2.A.-; 82.AA.-T | 1.721741871 | 0.598515022 |
| 2671130 | 716 | 2.A.C; 0.T.-; 85.TC.-G | 1.721255074 | 0.884259809 |
| 4200182 | 717 | 4.T.-; 55.-.G | 1.721190019 | 1.232924607 |
| 2281298 | 718 | 0.T.-; 86.-.G | 1.720150085 | 0.459949896 |

TABLE 8

| | SEQ | | | |
|---|---|---|---|---|
| index | ID NO | muts_1indexed | MI | 95% CI |
| 7182097 | 719 | 27.-.A; 77.GA.-- | 1.718675301 | 1.318350535 |
| 2251662 | 720 | 0.T.-; 74.T.- | 1.718536267 | 0.428185144 |
| 1904870 | 721 | 0.TTA.---; 3.C.A; | 1.715468512 | 1.34467556 |
| | | 76.G.- | | |
| 10553996 | 722 | 15.-.T; 81.GA.-T | 1.71542255 | 0.963037099 |
| 10202590 | 723 | 18.-.G; 73.A.- | 1.715117267 | 0.822174045 |
| 3028839 | 724 | 1.TA.--; 78.-.C | 1.712954587 | 0.450495404 |
| 3304552 | 725 | 0.T.-; 2.A.G; | 1.712919885 | 0.767193507 |
| | | 89.-.T | | |
| 4247308 | 726 | 4.T.-; 87.-.A | 1.711145921 | 0.765770921 |
| 4318521 | 727 | 4.T.-; 66.CT.-G | 1.710421741 | 0.956759562 |
| 7247759 | 728 | 27.-.C; 86.-.G | 1.709588646 | 1.198020951 |
| 10198320 | 729 | 18.-.G; 76.GG.-T | 1.709356476 | 0.700624761 |
| 2457655 | 730 | 1.TA.--; 3.C.A; | 1.709355062 | 1.259561047 |
| | | 76.GG.-C | | |
| 3032520 | 731 | 1.TA.--; 76.G.-; | 1.709186022 | 0.754280463 |
| | | 78.A.T | | |
| 2702792 | 732 | 0.T.-; 2.A.C; | 1.70908021 | 0.741854781 |
| | | 86.CC.-T | | |
| 12171374 | 733 | 2.A.-; 84.AT.-- | 1.708956084 | 1.239010302 |
| 10192666 | 734 | 18.-.G; 87.-.G | 1.706139319 | 0.672236416 |
| 2642318 | 735 | 2.A.C; 0.T.-; | 1.703389866 | 0.651239291 |
| | | 72.-.A | | |
| 2718074 | 736 | 2.A.C; 0.T.-; | 1.699976056 | 1.191093731 |
| | | 77.GA.--; 82.A.T | | |
| 12191670 | 737 | 2.A.-; 78.A.- | 1.696728454 | 0.819298298 |
| 2456219 | 738 | 1.TA.--; 3.C.A; | 1.696442704 | 1.260292211 |
| | | 74.T.- | | |
| 2457365 | 739 | 1.TA.--; 3.C.A; | 1.694881811 | 0.951237077 |
| | | 76.GG.-A | | |
| 8538180 | 740 | 75.-.G | 1.694861152 | 0.415924921 |
| 3020581 | 741 | 1.TA.--; | 1.692620071 | 1.160105308 |
| | | 86.CC.-T | | |
| 10281916 | 742 | 17.-.T; 76.-.A | 1.692603642 | 0.648841391 |
| 2707684 | 743 | 0.T.-; 2.A.C; | 1.691822732 | 1.346496086 |
| | | 82.A.-; 84.A.G | | |
| 2676761 | 744 | 0.T.-; 2.A.C; | 1.68930292 | 0.99991905 |
| | | 90.-.G | | |
| 7213979 | 745 | 27.G.-; 75.CG.-T | 1.688772312 | 1.195343004 |
| 2459101 | 746 | 1.TA.--; 3.C.A; | 1.686519606 | 0.966564286 |
| | | 77.GA-- | | |
| 8123571 | 747 | 75.-C; 86.-.C | 1.685647367 | 0.454380756 |
| 12207287 | 748 | 2.A.-; 75.CG.-T | 1.685305192 | 0.563871209 |
| 2740245 | 749 | 2.A.C; 0.T.-; | 1.684914398 | 1.012999566 |
| | | 70.-.T | | |
| 10531744 | 750 | 15.-.T; 88.G.- | 1.684556387 | 1.172453501 |
| 2669798 | 751 | 2.A.C; 0.T.-; | 1.683775918 | 0.485672655 |
| | | 82.-.A | | |
| 2294771 | 752 | 0.T.-; 78.-.T | 1.683554242 | 0.365785232 |
| 7213033 | 753 | 27.G.-; 76.GG.-T | 1.681704475 | 1.553533309 |
| 7829581 | 754 | 55.-.G; 76.G.- | 1.681581148 | 1.157922781 |
| 2808092 | 755 | 0.T.-; 2.A.C; | 1.680339253 | 1.570645735 |
| | | 28.-.T | | |
| 2960043 | 756 | 1.TA.--; 27.-.C | 1.675962289 | 1.352861328 |
| 10506564 | 757 | 15.-.T; 55.-.G | 1.675003018 | 1.443016487 |
| 4315349 | 758 | 4.T.-; 73.A.T | 1.667757548 | 0.705372587 |
| 2705067 | 759 | 2.A.C; 0.T.-; | 1.667686194 | 0.498039786 |
| | | 82.A.- | | |
| 3330280 | 760 | 0.T.-; 2.A.G; | 1.666946086 | 0.947896566 |
| | | 76.G.-; 78.A .T | | |
| 9630969 | 761 | 16.------------ . | 1.664680451 | 1.315435632 |
| | | CTCATTACTTTG; | | |
| | | 75.-.A | | |
| 12173513 | 762 | 2.A.-; 82.A.- | 1.663830201 | 0.733539657 |
| 3280346 | 763 | 0.T.-; 2.A.G; | 1.662631303 | 1.204381863 |
| | | 87.-.A | | |
| 7238549 | 764 | 27.-.C; 74.-.C | 1.661306709 | 1.214766158 |
| 8154695 | 765 | 76.G.-; 78.A.C | 1.661229303 | 0.368056731 |
| 10516784 | 766 | 15.-.T; 72.-.A | 1.66016215 | 0.597302394 |
| 10307953 | 767 | 17.-.T; 78.A.- | 1.65952488 | 0.82365406 |
| 12432835 | 768 | 1.TAC.---; 75.-.C | 1.654476204 | 0.813686317 |
| 12193344 | 769 | 2.A.-; 76.-.G | 1.653563552 | 0.663784021 |
| 2297191 | 770 | 0.T.-; 76.-.T | 1.652000897 | 0.458064366 |
| 2126158 | 771 | 0.TTA.---; | 1.649649089 | 1.318355451 |
| | | 3.C.G; 87.-G | | |
| 2283617 | 772 | 0.T.-; 83.-.C | 1.648963324 | 1.421238851 |
| 2654520 | 773 | 2.A.C; 0.T.-; | 1.647087379 | 0.573966628 |
| | | 75.CG.-A | | |
| 3332543 | 774 | 0.T.-; 2.A.G; | 1.644966768 | 0.844422969 |
| | | 76.-.T | | |
| 9604425 | 775 | 28.-.C88.G.- | 1.6439264 | 1.218234779 |
| 12109255 | 776 | 2.A.-; 73.-.A | 1.643507554 | 0.929692908 |
| 12438229 | 777 | 1.TAC.---; | 1.641912193 | 0.689368529 |
| | | 76.GG.-T | | |
| 8153054 | 778 | 77.G.C | 1.64142005 | 1.384906369 |
| 10308482 | 779 | 17.-.T; 76.-.G | 1.641323583 | 1.127042919 |
| 10300026 | 780 | 17.-.T; 86.C.- | 1.641224613 | 1.227957862 |
| 2715234 | 781 | 2.A.C; 0.T.-; | 1.640370122 | 1.47602933 |
| | | 80.AG.-C | | |
| 10532541 | 782 | 15.-.T; 90.T.- | 1.640240149 | 1.020337794 |
| 12721860 | 783 | 0.-.T; 76.G.- | 1.639509598 | 0.366635004 |
| 2460008 | 784 | 1.TA.--; 3.C.A; | 1.639261031 | 0.936045278 |
| | | 86.-.C | | |
| 2264044 | 785 | 0.T.-; 86.-.A | 1.639121471 | 0.511832699 |
| 12188811 | 786 | 2.A.-; 78.AG.-C | 1.637960122 | 0.77568855 |
| 12432569 | 787 | 1.TAC.---; | 1.637292013 | 0.882764983 |
| | | 76.GG.-A | | |
| 9602947 | 788 | 28.-.C; 75.-.C | 1.636117538 | 1.557596786 |
| 2994003 | 789 | 1.TA.--; 74. T.- | 1.633550393 | 0.541929003 |
| 12213405 | 790 | 2.A.-; 73.A.- | 1.63354167 | 0.735980135 |
| 2719575 | 791 | 0.T.-; 2.A.C; | 1.633437814 | 0.44613275 |
| | | 78.AG.-C | | |
| 2123173 | 792 | 0.TTA.---; 3.C.G; | 1.632290442 | 1.510924178 |
| | | 76.G.- | | |
| 10086342 | 793 | 19.-.T; 78.-.C | 1.630575414 | 0.477336939 |
| 12236371 | 794 | 2.A.-; 55.-.T | 1.629793154 | 0.850354697 |
| 6473588 | 795 | 16.-.C; 81.GA.-T | 1.6283178 | 0.397977937 |
| 7240999 | 796 | 27.-.C; 79.G.- | 1.627916832 | 1.310172414 |
| 12189370 | 797 | 2.A.-; 78.-.C | 1.625186884 | 0.714620198 |
| 3005003 | 798 | 1.TA.--; 85.TC.-G | 1.624844672 | 0.819992466 |
| 10185851 | 799 | 18.-.G; 86.-.C | 1.622189588 | 0.720091613 |
| 2725020 | 800 | 0.T.-; 2.A.C; | 1.621816405 | 0.69613073 |
| | | 78.AG.-T | | |

TABLE 9

| | SEQ ID | | | |
|---|---|---|---|---|
| index | NO | muts_1indexed | MI | 95% CI |
| 12212274 | 801 | 2.A.-; 70.-.T | 1.620710424 | 1.038198418 |
| 8470264 | 802 | 78.-.C | 1.617470851 | 0.271680388 |
| 2286841 | 803 | 0.T.-; 82.AA.-G | 1.617088496 | 0.606230824 |
| 7241506 | 804 | 27.-.C; 81.GA.-C | 1.616908898 | 1.111991942 |
| 12163987 | 805 | 2.A.-; 89.A.G | 1.616843955 | 0.718476436 |
| 3364655 | 806 | 0.T.-; 2.A.G; | 1.615459441 | 1.131392113 |
| | | 55.-.T | | |
| 1904677 | 807 | 0.TTA.---; 3.C.A; | 1.613614518 | 0.965094427 |
| | | 75.-.C | | |
| 2712438 | 808 | 2.A.C; 0.T.-; 82.-.T | 1.61208488 | 0.769494423 |
| 14645004 | 809 | -29.A.C; 0.T.-; | 1.610092293 | 0.432743672 |
| | | 2.A.C; 76.G.- | | |
| 10322550 | 810 | 17.-.T; 55.-.T | 1.608294231 | 0.835345091 |
| 10304965 | 811 | 17.-.T; 82.AA.-T | 1.605684059 | 1.005872373 |
| 10279228 | 812 | 17.-.T; 74.-.C | 1.603403686 | 0.964621553 |
| 3263089 | 813 | 2.A.G; 0.T.-; | 1.603002415 | 0.944419565 |
| | | 74.-.G | | |
| 2282393 | 814 | 0.T.-; 82.A.-; | 1.601545542 | 1.047011173 |
| | | 85.T.G | | |
| 2463251 | 815 | 1.TA .--; 3.C.A; | 1.597766756 | 0.958863507 |
| | | 86.C.- | | |
| 2459897 | 816 | 1.TA .--; | 1.595799757 | 0.724801659 |
| | | 3.C.A; 88.G.- | | |
| 1852430 | 817 | 0.TT.--; 76.GG.-A | 1.595672352 | 0.848408617 |
| 10305251 | 818 | 17.-.T; 81.GA.-T | 1.593404575 | 1.07855471 |
| 9603994 | 819 | 28.-.C; 85.TC.-A | 1.593398609 | 1.338922574 |
| 4319798 | 820 | 4.T.-; 66.CT.-- | 1.5927753 | 0.719209709 |
| 3042484 | 821 | 1 .TA.--; 66.CT.-G | 1.592062494 | 0.578104998 |
| 8544184 | 822 | 75.-.G; 87.-.T | 1.591574219 | 0.630898033 |
| 2709867 | 823 | 2.A.C; 0.T.-; | 1.590223625 | 0.505705027 |
| | | 82.AA.-C | | |
| 3439310 | 824 | 0.T.-; 2.A.G; | 1.589266839 | 0.341479677 |
| | | 15.-.T | | |
| 2718364 | 825 | 0.T.-; 2.A.C; | 1.587566696 | 1.149184797 |
| | | 80.A.T | | |
| 4223967 | 826 | 4.T.-; 73.-.A | 1.587282349 | 0.645700343 |
| 4271617 | 827 | 4.T.-; 89.AT.-G | 1.587137334 | 1.233444621 |
| 10460510 | 828 | 16.C.-; 76.GG.-A | 1.586590153 | 0.787644542 |
| 4227764 | 829 | 4.T.-; 74.-.G | 1.585660861 | 0.680124313 |
| 9994855 | 830 | 19.-.G; 76.GG.-T | 1.58530649 | 0.779320174 |
| 3272821 | 831 | 2.A.G; 0.T.-; | 1.583120825 | 0.912440621 |
| | | 76.G.-; 78.A.C | | |
| 12110798 | 832 | 2.A.-; 74.T.- | 1.581717864 | 0.658647546 |
| 1975319 | 833 | 0.T.C; 76.G.- | 1.58114814 | 0.609951036 |
| 10316332 | 834 | 17.-.T; 73.A.- | 1.580871543 | 0.902426494 |
| 2720616 | 835 | 0.T.-; 2.A.C; | 1.58077409 | 0.565168836 |
| | | 78.A.C | | |
| 8753785 | 836 | 55.-.T; 86.-.C | 1.580570661 | 0.907594533 |
| 8112378 | 837 | 76.-.A | 1.579846517 | 0.965148419 |
| 2819005 | 838 | 0.T.-; 2.A.C; | 1.579281152 | 0.490774802 |
| | | 18.-.G | | |
| 8357828 | 839 | 87.-.G | 1.578903423 | 0.260894611 |
| 6477023 | 840 | 16.-.C; 76.GG.-T | 1.577281377 | 0.801993714 |
| 12737747 | 841 | 0.-.T; 87.-.G | 1.576853785 | 0.587015792 |
| 12309294 | 842 | 2.A.-; 17.-.T | 1.575651742 | 0.644197096 |
| 2252133 | 843 | 0.T.-; 74.-.C | 1.575512867 | 0.340117554 |
| 10567192 | 844 | 15.-.T; 73.AT.-G | 1.575291887 | 0.657147067 |
| 3261438 | 845 | 2.A.G; 0.T.-; 74.-.C | 1.574575619 | 0.783331617 |
| 15169229 | 846 | -29.A.G; 75.-.G | 1.574259504 | 0.382115947 |
| 6128804 | 847 | 14.-.A; | 1.573502126 | 0.97997063 |
| | | 76.GG.-T | | |
| 12197720 | 848 | 2.A.-; 76.G.-; | 1.57327628 | 0.892867309 |
| | | 78.A.T | | |
| 3326919 | 849 | 2.A.G; 0.T.-; | 1.572520314 | 0.782894375 |
| | | 76.-.G | | |
| 12164376 | 850 | 2.A.-; 89.A.- | 1.571939028 | 1.399860294 |
| 2990209 | 851 | 1.TA.--; 70.T.- | 1.571341225 | 1.473641775 |
| 8538220 | 852 | 75.-.G; 132.G.T | 1.5708167 | 0.464722537 |
| 10068467 | 853 | 19.-.T; 76.GG.-A | 1.570115611 | 0.903671278 |
| 9697533 | 854 | 28.-.T; 75.CG.-T | 1.568984808 | 1.329590045 |
| 2958993 | 855 | 1.TA.--; 27.-.A | 1.567973804 | 1.255119149 |
| 3001629 | 856 | 1 .TA.--; 76.G.-; | 1.566060562 | 0.524342191 |
| | | 78.A.C | | |
| 4291732 | 857 | 4.T.-; 77.GA.--; | 1.564592325 | 1.309941389 |
| | | 82.A.T | | |
| 4238868 | 858 | 4.T.-; 76.G.-; | 1.56447294 | 0.829602825 |
| | | 78.A.C | | |
| 3306461 | 859 | 0.T.-; 2.A.G; | 1.563833782 | 0.717413376 |
| | | 87.-.G | | |
| 1937976 | 860 | 2.A.C; 0.TT.--; | 1.560038457 | 1.462696008 |
| | | 76.G.- | | |
| 4172716 | 861 | 4.T.-; 27.-.C | 1.558070079 | 1.387693861 |
| 12185288 | 862 | 2.A.-; 80.A.- | 1.557024858 | 0.705941145 |
| 14813579 | 863 | -29.A.C; 75.-.G | 1.556839809 | 0.414912384 |
| 2468675 | 864 | 1.TA.--; 3.C.A; | 1.553046656 | 0.931035197 |
| | | 75.CG.-T | | |
| 12195510 | 865 | 2.A.-; 78.AG.-T | 1.55000419 | 0.886783857 |
| 4285997 | 866 | 4.T.-; 82.AA.-G | 1.549250991 | 0.782347429 |
| 3275841 | 867 | 2.A.G; 0.T.-; | 1.549221581 | 0.526146695 |
| | | 77.GA.-- | | |
| 3018032 | 868 | 1.TA.--; 89.A.- | 1.549009371 | 1.113927175 |
| 2301817 | 869 | 0.T.-; 73.A.C | 1.54864254 | 0.917412432 |
| 3305057 | 870 | 0.T.-; 2.A.G; 88.-.T | 1.547965444 | 0.420214747 |
| 2122618 | 871 | 0.TTA.---; 3.C.G; | 1.547889984 | 1.094378143 |
| | | 76.GG.-A | | |
| 2289325 | 872 | 0.T.-; 80.A.- | 1.547099084 | 0.393404706 |
| 4291562 | 873 | 4.T.-; 80.AG.-T | 1.546888356 | 1.017074272 |
| 10557226 | 874 | 15.-.T; 78.-.C | 1.544857428 | 0.974814633 |
| 12748115 | 875 | 0.-.T; 76.GG -T | 1.544686324 | 0.709928076 |
| 3026518 | 876 | 1.TA.--; 80.AG.-C | 1.544042546 | 1.240581963 |
| 10545028 | 877 | 15.-.T; 89.-.C | 1.542272906 | 0.579291446 |
| 3416823 | 878 | 0.T.-; 2.A.G; 28.-.C | 1.53913175 | 1.436213329 |
| 9976094 | 879 | 19.-.G; 76.G.- | 1.538689261 | 0.748851507 |
| 1852751 | 880 | 0.TT.--; 76.GG.-C | 1.536921551 | 0.769662735 |
| 4314686 | 881 | 4.T.-; 73.A.- | 1.536187783 | 1.014477961 |

TABLE 10

| | SEQ ID | | | |
|---|---|---|---|---|
| index | NO | muts_1indexed | MI | 95% CI |
| 6470272 | 882 | 16.-.C; 87.-.T | 1.535725631 | 0.59665986 |
| 2673006 | 883 | 0.T.-; 2.A.C; | 1.535462742 | 0.804157995 |
| | | 87.C.A | | |
| 12137377 | 884 | 2.A.-; 86.-.C | 1.535147851 | 0.546194055 |
| 12184036 | 885 | 2.A.-; 80.AG.-C | 1.531564715 | 1.351567783 |
| 10285242 | 886 | 17.-.T; 77.-.C | 1.53026457 | 1.164347551 |
| 2263017 | 887 | 0.T.-; 82.-.A | 1.529811403 | 0.467986989 |
| 12163286 | 888 | 2.A.-; 89.AT.-G | 1.528822089 | 1.00107691 |
| 2706481 | 889 | 2.A.C; 0.T.-; | 1.52754828 | 1.209383598 |
| | | 82.A.-; 84.A.C | | |
| 4320578 | 890 | 4.T.-; 66.C.- | 1.527179936 | 0.994611388 |
| 3004121 | 891 | 1.TA.--; 85.TC.-A | 1.525870388 | 0.697533949 |
| 3269260 | 892 | 2.A.G; 0.T.-; 75.-.C | 1.521722305 | 0.738666566 |
| 7835518 | 893 | 55.-.G; 76.-.G | 1.518881805 | 0.935071683 |
| 10195401 | 894 | 18.-.G; 81.GA.-T | 1.518543539 | 0.775808631 |
| 6477333 | 895 | 16.-.C; 76.-.T | 1.51587769 | 0.626814313 |
| 4171307 | 896 | 4.T.-; 27.-.A | 1.513605325 | 1.233769066 |
| 10299590 | 897 | 17.-.T; 88.-.T | 1.513069933 | 1.295754832 |
| 6478447 | 898 | 16.-.C; 75.C.- | 1.512491339 | 0.508038646 |
| 4249490 | 899 | 4.T.-; 88.GA.-C | 1.512130404 | 0.73669735 |
| 12220656 | 900 | 2.A.-; 66.C.- | 1.512020037 | 1.05546421 |
| 7240739 | 901 | 27.-.C; 77.-.A | 1.511778431 | 1.177553371 |
| 10315246 | 902 | 17.-.T; 73.AT.-G | 1.511330905 | 1.009774993 |
| 1944754 | 903 | 0TT.--; 2.A.C; | 1.511225805 | 1.155505022 |
| | | 76.-.G | | |
| 3337255 | 904 | 2.A.G; 0.T.-; 74.-.T | 1.509602507 | 0.678006083 |
| 6362999 | 905 | 17.-.A; 76.G.- | 1.508590435 | 1.042551324 |
| 3017407 | 906 | 1.TA.--; 89.-.C | 1.508577828 | 0.465448085 |
| 9973601 | 907 | 19.-.G; 75.-.A | 1.502907348 | 0.893737423 |
| 12186826 | 908 | 2.A.-; 80.AG.-T | 1.500547059 | 0.812595989 |
| 3035711 | 909 | 1.TA.--; 75.C.- | 1.50008318 | 0.591995026 |
| 8526584 | 910 | 76.-.T | 1.499331872 | 0.320393064 |
| 2211100 | 911 | 0.T.-; 27.-.A | 1.498766744 | 1.299978621 |
| 8558515 | 912 | 74.-.T | 1.498532736 | 0.244304059 |
| 4321895 | 913 | 4.T.-; 65.GC.-T | 1.498442707 | 0.661273129 |
| 12204638 | 914 | 2.A.-; 75.C.- | 1.49596065 | 0.654918883 |
| 8118238 | 915 | 76.GG.-C | 1.495070866 | 0.554503755 |
| 2348592 | 916 | 0.T.-; 19.-.T | 1.493134598 | 0.463440478 |
| 3282394 | 917 | 0.T.-; 2.A.G; | 1.490851105 | 1.143853171 |
| | | 88.GA.-C | | |
| 9974216 | 918 | 19.-.G; 76.GG.-A | 1.489833949 | 0.650334517 |
| 3435006 | 919 | 0.T.-; 2.A.G; | 1.487780343 | 0.572012417 |
| | | 17.-.T | | |
| 2291281 | 920 | 0.T.-; 78.AG.-C | 1.48644962 | 0.721753764 |
| 3013663 | 921 | 1.TA.--; 99.-.G | 1.484001366 | 0.730348567 |
| 7255023 | 922 | 27.-.C; 70.-.T | 1.483723737 | 1.383884246 |
| 4307384 | 923 | 4.T.-; 75.C.- | 1.483251669 | 0.591919226 |
| 2702279 | 924 | 0.T.-; 2.A.C; | 1.482180584 | 1.154754969 |
| | | 86.CC.-G | | |
| 3036396 | 925 | 1.TA.--; 74.-.T | 1.480425433 | 0.455235967 |
| 10196645 | 926 | 18.-.G; 78.-.C | 1.478934738 | 0.7577364 |
| 4308690 | 927 | 4. T.-74.-.T | 1.478644519 | 0.955354495 |
| 4298804 | 928 | 4.T.-; 78.A.G | 1.476605159 | 0.725427219 |
| 12125860 | 929 | 2.A.-; 76.G.-; | 1.47599621 | 0.782159575 |
| | | 78.A.C | | |
| 2675530 | 930 | 0.T.-; 2.A.C; | 1.473977708 | 1.266428954 |
| | | 90.T.- | | |
| 7242260 | 931 | 27.-.C; 88.G.- | 1.473373043 | 1.439338655 |
| 4287312 | 932 | 4.T.-; 82.AA.-T | 1.472766154 | 0.577453742 |
| 3339492 | 933 | 2.A.G; 0.T.-; | 1.471548367 | 1.444939954 |
| | | 73.AT.-C | | |
| 4290113 | 934 | 4.T.-; 80.A.- | 1.470113687 | 0.639199692 |
| 2293835 | 935 | 0.T.-; 78.A.-; 80.A.- | 1.469388611 | 0.86669662 |
| 6455860 | 936 | 16.-.C; 74.-.C | 1.467963371 | 0.526897826 |
| 2706303 | 937 | 0.T.-; 2.A.C; | 1.467184493 | 1.023191849 |
| | | 82.AA.--; 85.T.C | | |
| 7252350 | 938 | 27.-.C; 76.-.T | 1.467027327 | 1.179599877 |
| 3277392 | 939 | 0.T.-; 2.A.G; | 1.466923265 | 1.201147414 |
| | | 85.TC.-A | | |
| 8538161 | 940 | 75.-.G; 132.G.C | 1.466591325 | 0.427589068 |
| 8202442 | 941 | 87.-.A | 1.464924451 | 0.818791149 |
| 2898633 | 942 | 1.-.C; 78.-.C | 1.464030898 | 0.456291529 |
| 2648767 | 943 | 2.A.C; 0.T.-; 73.-.A | 1.463173362 | 0.658913335 |
| 6115163 | 944 | 14.-.A; 88.G.- | 1.46294421 | 0.52938306 |
| 10576534 | 945 | 15.-.T; 55.-.T | 1.461210677 | 0.556416566 |
| 1904556 | 946 | 0.TTA.---; 3.C.A; | 1.461144948 | 1.088815589 |
| | | 76.GG.-C | | |
| 8073267 | 947 | 74.-.C | 1.458640802 | 0.430303917 |
| 8755280 | 948 | 55.-.T | 1.458287413 | 0.637579805 |
| 2341059 | 949 | 0.T.-; 28.-.C | 1.457350597 | 1.284432147 |
| 3007006 | 950 | 1.TA.--; 90.T.- | 1.45647646 | 1.125399861 |
| 7833962 | 951 | 55.-.G; 87.-.G | 1.456238024 | 0.883248585 |
| 4299868 | 952 | 4.T.-; 78.-.T | 1.455724565 | 0.940309293 |
| 8342692 | 953 | 89.A.G | 1.454833967 | 0.974687875 |
| 2262741 | 954 | 0.T.-; 85.TC.-A | 1.451410557 | 0.583323465 |
| 1942088 | 955 | 0TT.--; 2.A.C; | 1.450492391 | 1.215838114 |
| | | 86.C.- | | |
| 10200245 | 956 | 18.-.G; 74.-.T | 1.448405766 | 0.937707192 |
| 4219211 | 957 | 4.T.-; 72.-.A | 1.446520177 | 0.549344991 |
| 2457931 | 958 | 1.TA.--; 3.C.A; | 1.444076731 | 0.735893179 |
| | | 75.-.C | | |
| 3038631 | 959 | 1.TA.--; 73.AT.-G | 1.443584213 | 0.559939739 |
| 12753950 | 960 | 0.-.T; 73.A.- | 1.4435332 | 0.573037517 |
| 2129014 | 961 | 0.TTA.---; 3.C.G; | 1.439545748 | 1.366024853 |
| | | 75.-.G | | |
| 7833901 | 962 | 55.-.G; 86.C.- | 1.439456801 | 0.67108624 |
| 10066878 | 963 | 19.-.T; 74.-.C | 1.43944975 | 0.662912873 |

TABLE 11

| | SEQ | | | |
|---|---|---|---|---|
| index | ID NO | muts_1indexed | MI | 95% CI |
| 2714726 | 964 | 0.T.-; 2.A.C; | 1.438502347 | 0.738791942 |
| | | 77.GA.--; 83.A.T | | |
| 12106738 | 965 | 2.A.-; 72.-.G | 1.437789303 | 1.200787575 |
| 2720418 | 966 | 0.T.-; 2.A.C; | 1.43644621 | 1.201219979 |
| | | 77.GA.--; 80.A.C | | |
| 2291924 | 967 | 0.T.-; 78.A.C | 1.4359349 | 0.93677707 |
| 9991025 | 968 | 19.-.G; 81.GA.-T | 1.434371779 | 0.688279351 |
| 4243954 | 969 | 4.T.-; 85.TC.-A | 1.432539899 | 0.673581956 |
| 6362816 | 970 | 17.-.A; 75.-.C | 1.432516289 | 0.887237626 |
| 8204227 | 971 | 87.C.A | 1.432133272 | 1.064542809 |
| 1980019 | 972 | 0.T.C; 78.A.- | 1.431187129 | 0.702091337 |
| 8142815 | 973 | 76.G.-; 130.T.G | 1.429104435 | 0.270795433 |
| 10554966 | 974 | 15.-.T; 80.A.- | 1.428888329 | 1.003322663 |
| 2702620 | 975 | 0.T.-; 2.A.C; | 1.427340154 | 0.891520531 |
| | | 86.C.T | | |
| 8142856 | 976 | 76.G.-; 132.G.C | 1.427043687 | 0.237774998 |
| 12012995 | 977 | 2.A.-; 16.-.C | 1.424513327 | 0.515408648 |
| 4284095 | 978 | 4.T.-; 82.AA.-C | 1.424103366 | 0.718417545 |
| 10546168 | 979 | 15.-.T; 88.-.T | 1.423883538 | 1.002262718 |
| 8128579 | 980 | 75.-.C | 1.423710515 | 0.273255106 |
| 2703946 | 981 | 2.A.C; 0.T.-; | 1.423451845 | 1.275687556 |
| | | 82.A.-; 85.T.G | | |
| 12433040 | 982 | 1.TAC.---; 76.G.- | 1.422927656 | 0.851734633 |
| 12162901 | 983 | 2.A.-; 89.-.C | 1.42171048 | 0.831363626 |
| 2814556 | 984 | 0.T.-; 2.A.C; 19.-.G | 1.420198732 | 0.571931257 |
| 8142933 | 985 | 76.G.-; 132.G.T | 1.41986544 | 0.297329476 |
| 2710592 | 986 | 2.A.C; 0.T.-; 81.-.G | 1.419787754 | 0.684050276 |
| 8537382 | 987 | 75.-.G; 121.C.A | 1.419392503 | 0.407819009 |
| 12434064 | 988 | 1.TAC.---; 86.-.C | 1.417035784 | 0.739250344 |
| 12438652 | 989 | 1. TAC.---; 75.C.- | 1.416797803 | 0.893829093 |
| 8105679 | 990 | 76.GG.-A | 1.415509749 | 0.237573505 |
| 8089861 | 991 | 75.-.A; 86.-.C | 1.414086312 | 0.397272867 |
| 10177945 | 992 | 18.-.G; 72.-.A | 1.413781205 | 0.836300188 |
| 4243445 | 993 | 4.T.-; 81.GA.-C | 1.413254084 | 0.887148369 |
| 8123491 | 994 | 75.-.C; 88.G.- | 1.41240947 | 0.440956817 |
| 4313666 | 995 | 4.T.-; 70.-.T | 1.411481565 | 0.506158491 |
| 7180551 | 996 | 27.-.A; 76.-.A | 1.409575725 | 1.180673384 |
| 6534510 | 997 | 17.-.G; 76.GG.-T | 1.407215614 | 0.941339052 |
| 3025550 | 998 | 1.TA.--; 82.AA.-T | 1.406508777 | 0.569736842 |
| 10275000 | 999 | 17.-.T; 71.-.C | 1.40607729 | 0.754323892 |
| 8530347 | 1000 | 75.-C.GA | 1.405553591 | 0.332518861 |
| 12438782 | 1001 | 1.TAC.---; 74.-.T | 1.404014328 | 0.86810435 |
| 2724111 | 1002 | 2.A.C; 0.T.-; 78.A.-; | 1.402948435 | 1.013377956 |
| | | -80.A. | | |
| 12682492 | 1003 | 0.-.T; 27.-.C | 1.402481385 | 1.265768183 |
| 8336449 | 1004 | 89.-.C | 1.399968085 | 0.251375019 |
| 2994450 | 1005 | 1.TA.--; 74.-.C | 1.399303097 | 0.436372549 |
| 10070026 | 1006 | 19.-.T; 76.G.- | 1.398597697 | 0.599022476 |
| 4246898 | 1007 | 4.T.-; 86.CC.-A | 1.398315453 | 0.996312871 |
| 2056199 | 1008 | 0TT.--; 2.A.G; | 1.397796768 | 1.058988953 |
| | | 82.AA.-T | | |
| 2726405 | 1009 | 0.T.-; 2.A.C; | 1.397727971 | 0.988558899 |
| | | 77.G.T | | |
| 8093322 | 1010 | 75.-.A | 1.396233471 | 0.309278367 |
| 4239175 | 1011 | 4.T.-; 77.-.C | 1.395763792 | 0.978685252 |
| 3031832 | 1012 | 1.TA.--; 78.-.T | 1.394964503 | 0.529438738 |
| 2303944 | 1013 | 0.T.-; 73.A.- | 1.394767477 | 0.685653215 |
| 2255406 | 1014 | 0.T.-; 76.GG.-- | 1.39467151 | 1.055424187 |
| 2468522 | 1015 | 1.TA.--; 3.C.A; | 1.393765331 | 0.747608286 |
| | | 74.-.T | | |
| 8543995 | 1016 | 75.-.G; 86.C.- | 1.39257441 | 0.371930382 |
| 8348831 | 1017 | 88.-.T | 1.392335932 | 0.333299943 |
| 2899043 | 1018 | 1.-.C; 78.A.- | 1.392119807 | 0.692690413 |
| 6611143 | 1019 | 18.C.-; 75.-.A | 1.391822496 | 0.602240717 |
| 8142880 | 1020 | 76.G.- | 1.39077182 | 0.256141665 |
| 4294538 | 1021 | 4.T.-; 78.A.C | 1.390406199 | 0.607275427 |
| 447196 | 1022 | -27.C.A; 75.-.G | 1.390265949 | 0.365279208 |
| 3338210 | 1023 | 2.A.G; 0.T.-; | 1.390242773 | 0.685982978 |
| | | 75.CG.-T | | |
| 8538250 | 1024 | 75.-.G; 131.A.C | 1.389343955 | 0.441726963 |
| 10302419 | 1025 | 17.-.T; 83.-.C | 1.388447653 | 1.345445476 |
| 3169133 | 1026 | 0.T.-; 2.A.G; | 1.387799855 | 0.626570598 |
| | | 16.-.C | | |
| 1855234 | 1027 | 0.TT.--; 86.-.C | 1.386552663 | 0.590192706 |
| 3027053 | 1028 | 1.TA.--; 80.A.- | 1.386335615 | 0.44423395 |
| 8142905 | 1029 | 76.G.-; 133.A.C | 1.386299403 | 0.311670925 |
| 2465375 | 1030 | 1.TA.--; 3.C.A; | 1.386188008 | 0.849600498 |
| | | 81.GA.-T | | |
| 8137397 | 1031 | 76.G.-; 98.-.A | 1.38509752 | 0.65791826 |
| 3304306 | 1032 | 2.A.G; 0.T.-; | 1.38362179 | 1.225993381 |
| | | 89.A.- | | |
| 8537231 | 1033 | 75.-.G; 120.C.A | 1.383053376 | 0.450967918 |
| 4299393 | 1034 | 4.T.-; 78.AG.-T | 1.382187217 | 1.034357685 |
| 3295454 | 1035 | 2.A.G; 0.T.-; | 1.381863603 | 1.038871163 |
| | | 99.-.G | | |
| 8519489 | 1036 | 76.GG.-T | 1.379556363 | 0.163945711 |
| 3264318 | 1037 | 2.A.G; 0.T.-; | 1.379358937 | 0.702823304 |
| | | 75.-.A | | |
| 3266116 | 1038 | 2.A.G; 0.T.-; | 1.379046637 | 0.672325549 |
| | | 76.GG.-A | | |
| 2997992 | 1039 | 1.TA.--; 76.-.A | 1.378072319 | 0.700284634 |
| 2672282 | 1040 | 2.A.C; 0.T.-; | 1.376499067 | 0.804782737 |
| | | 86.CC.-A | | |
| 14798941 | 1041 | -29.A.C; 75.-.C | 1.375822882 | 0.254844812 |
| 12031760 | 1042 | 2.A.-; 27.G.- | 1.375192693 | 1.374595871 |
| 2201185 | 1043 | 0.T.-; 16.-.C | 1.372900924 | 0.445813321 |
| 2400173 | 1044 | 1.-.A; 76.G.- | 1.372064456 | 0.596118731 |
| 10088256 | 1045 | 19.-.T; 76.G.-; | 1.369986019 | 0.714603396 |
| | | 78.A.T | | |
| 10284913 | 1046 | 17 -.T; 77.- A | 1.369839502 | 1.090311599 |

TABLE 12

| | SEQ | | | |
|---|---|---|---|---|
| index | ID NO | muts_1indexed | MI | 95% CI |
| 10545701 | 1047 | 15.-.T; 89.A.- | 1.369748818 | 1.003332985 |
| 8212851 | 1048 | 86.-.C | 1.369391509 | 0.539620134 |
| 8132895 | 1049 | 75.-.C; 86.C.- | 1.368039243 | 0.296779105 |
| 3281950 | 1050 | 2.A.G; 0.T.-; | 1.367611373 | 0.907291353 |
| | | 86.-.C | | |
| 1858655 | 1051 | 0.TT.--; 87.-.G | 1.367558992 | 0.620186488 |
| 12737396 | 1052 | 0.-.T; 86.C.- | 1.365343254 | 0.552234176 |
| 6474033 | 1053 | 16.-.C; 80.A.- | 1.363437029 | 0.56174258 |
| 2646406 | 1054 | 0.T.-; 2.A.C; | 1.36343607 | 1.115304879 |
| | | 72.-.G | | |
| 3020097 | 1055 | 1.TA.--; 86.-.G | 1.363355265 | 0.580106368 |
| 12160739 | 1056 | 2.A.-; 91.A.-; | 1.363329423 | 1.066828539 |
| | | 93.A.G | | |
| 14919005 | 1057 | -29.A.C; 2.A.-; | 1.362482864 | 0.432898468 |
| | | 76.G.- | | |
| 10527714 | 1058 | 15.-.T; 79.G.- | 1.361775897 | 0.846824969 |
| 3023033 | 1059 | 1.TA.--; 82.A.-; | 1.361357615 | 1.194817135 |
| | | 84.A.G | | |
| 2467773 | 1060 | 1.TA.--; 3.C.A; | 1.36121818 | 0.679797788 |
| | | 76.-.T | | |
| 2284824 | 1061 | 0.T.-83.-.T | 1.360543389 | 0.848033047 |
| 9987305 | 1062 | 19.-.G; 87.-.G | 1.360442144 | 0.734418526 |
| 2628450 | 1063 | 2.A.C; 0.T.-; | 1.360069277 | 0.861447129 |
| | | 65.GC.-A | | |
| 8531228 | 1064 | 75.-.G; 87.-.A | 1.359545621 | 0.690949702 |
| 1939243 | 1065 | 0.TT.--; 2.A.C; | 1.358280955 | 0.943115167 |
| | | 86.-C | | |
| 3050495 | 1066 | 1.TA.--; 55.-.T | 1.358171094 | 0.87966165 |
| 7835450 | 1067 | 55.-.G; 78.A.- | 1358033334 | 0.698343089 |
| 12702721 | 1068 | 0.-.T; 55.-.G | 1.357295007 | 0.530874809 |
| 4231994 | 1069 | 4.T.-; 76.-.A | 1.357045893 | 0.79932847 |
| 10185683 | 1070 | 18.-.G; 88.G.- | 1.35658647 | 1.037901 |
| 2709497 | 1071 | 2.A.C; 0.T.-; | 1.355764778 | 1.203503878 |
| | | 82.A.C | | |
| 8330844 | 1072 | 91.A.G | 1.355287946 | 1.033211677 |
| 10287644 | 1073 | 17.-.T; 85.TC.-G | 1.355153586 | 1.18231053 |
| 9976346 | 1074 | 19.-.G; 77.-.A | 1.354948471 | 0.743583366 |
| 8759277 | 1075 | 55.-.T; 75.-.G | 1.352910748 | 0.800352238 |
| 2711676 | 1076 | 2.A.C; 0.T.-; | 1.351869067 | 0.771861665 |
| | | 82.AA.-G | | |
| 10199887 | 1077 | 18.-.G; 75.C.- | 1.351414349 | 0.818440979 |
| 12131652 | 1078 | 2.A.-; 85.TC.-A | 1.351255788 | 1.139173311 |
| 8628479 | 1079 | 66.CT.-G; 76.G.- | 1.350688923 | 0.362115272 |
| 2459762 | 1080 | 1.TA.--; 3.C.A; | 1.350298722 | 1.009173521 |
| | | 87.-.A | | |
| 8647329 | 1081 | 66.C.T | 1.350057167 | 1.188259683 |
| 6526262 | 1082 | 17.-.G; 76.G.- | 1.349925914 | 1.264875753 |
| 2279498 | 1083 | 0.T.-; 88.-.T | 1.349921712 | 0.487773646 |
| 2719218 | 1084 | 0.T.-; 2.A.C; 79. | 1.349444156 | 1.087166266 |
| | | GAGAAA.TTTCTC | | |
| 1858516 | 1085 | 0.TT.--; 86.C.- | 1.349395537 | 1.336682614 |
| 14798574 | 1086 | -29.A.C; 76.GG.-C | 1.34699507 | 0.500207927 |
| 10178596 | 1087 | 18.-.G; 72.-.C | 1.346450015 | 0.765748852 |
| 8118222 | 1088 | 76.GG.-C; 132.G.C | 1.34615675 | 0.516935159 |
| 12181387 | 1089 | 2.A.-; 82.-.T | 1.344913969 | 0.639139505 |
| 10285141 | 1090 | 17.-.T; 76.G.-; | 1.344831557 | 0.980116215 |
| | | 78.A.C | | |
| 8565359 | 1091 | 75.CG.-T | 1.344784065 | 0.28783714 |
| 8142963 | 1092 | 76.G.-; 131.A.C | 1.344489963 | 0.258971589 |
| 6313836 | 1093 | 16.-.A; 78.A.- | 1.341546233 | 0.715419964 |
| 6455586 | 1094 | 16.-.C; 74.T.- | 1.340536921 | 0.588962188 |
| 10069022 | 1095 | 19.-.T; 76.GG.-C | 1.339199983 | 0.689265401 |
| 8538125 | 1096 | 75.-.G; 130.T.G | 1.339090974 | 0.405488829 |
| 8208034 | 1097 | 88.G.- | 1.339014146 | 0.22663535 |
| 4210228 | 1098 | 4.T.-; 65.G.- | 1.337504821 | 0.725776958 |
| 8555144 | 1099 | 74.-.T; 86.-.C | 1.336356371 | 0.495439384 |
| 2211631 | 1100 | 0.T.-; 27.G.- | 1.335840597 | 1.02295738 |
| 14799468 | 1101 | -29.A.C; 76.G.- | 1.335226973 | 0.265255991 |
| 3023524 | 1102 | 1.TA.--; 82.AA.-- | 1.334715286 | 0.777258592 |
| 14921453 | 1103 | -29.A.C; 2.A.-; | 1.334084702 | 0.448087214 |
| | | 75.-.G | | |
| 2465666 | 1104 | 1.TA.--; 3.C.A; | 1.333777233 | 1.225453831 |
| | | 80.A.-- | | |
| 2124272 | 1105 | 0.TTA.---; 3.C.G; | 1.333161176 | 1.020991136 |
| | | 86.-.C | | |
| 4366553 | 1106 | 4.T.-; 28.-.C | 1.333118117 | 1.147457336 |
| 15160651 | 1107 | -29.A.G; 75.-.C | 1.332785693 | 0.280235081 |
| 2248937 | 1108 | 0.T.-; 70.T.-; 73.A.C | 1.329283638 | 1.288981376 |
| 10307622 | 1109 | 17.-.T; 78.A.C | 1.328660147 | 0.893411396 |
| 2670634 | 1110 | 0.T.-; 2.A.C; | 1.327285114 | 0.860888625 |
| | | 85.TC.-- | | |
| 10180147 | 1111 | 18.-.G; 74.-.C | 1.326125292 | 0.932899353 |
| 10288203 | 1112 | 17.-.T; 87.-.A | 1.325075156 | 0.741328018 |
| 14806896 | 1113 | -29.A.C; 87.-.G | 1.324442672 | 0.255955368 |
| 2708627 | 1114 | 0.T.-; 2.A.C; | 1.32346629 | 0.575802358 |
| | | 82.AA.- | | |
| 3260655 | 1115 | 2.A.G; 0.T.-; 74.T.- | 1.322242725 | 0.641221404 |
| 12719454 | 1116 | 0.-.T; 76.GG.-A | 1.322124436 | 0.483164367 |
| 12432022 | 1117 | 1.TAC.---; 74.-.C | 1.320938397 | 0.64685233 |
| 4245923 | 1118 | 4.T.-; 85.TC.-G | 1.320596842 | 1.255360283 |
| 8363261 | 1119 | 87.-.T | 1.320550533 | 0.482292904 |
| 2128723 | 1120 | 0.TTA.---; | 1.318357676 | 1.198530269 |
| | | 3.C.G; 76.GG.-T | | |
| 8514493 | 1121 | 77.-.T | 1.317772824 | 0.80389443 |
| 3330625 | 1122 | 0.T.-; 2.A.G; | 1.317088275 | 1.251882713 |
| | | 77.-.T | | |
| 10279842 | 1123 | 17.-.T; 74.-.G | 1.316219704 | 0.99735284 |
| 3271300 | 1124 | 2.A.G; 0.T.-; | 1.315040838 | 0.602125183 |
| | | 76.G.- | | |
| 12209957 | 1125 | 2.A.-; 73.-.G | 1.314239351 | 1.123034513 |
| 2295677 | 1126 | 0.T.-; 76.G.-; | 1.313626293 | 0.643771948 |
| | | 78.A.T | | |
| 7188615 | 1127 | 27.-.A; 79. | 1.311956522 | 1.250658747 |
| | | GAGAAA.TTTCTC | | |

TABLE 13

| | SEQ | | | |
|---|---|---|---|---|
| index | ID NO | muts_1indexed | MI | 95% CI |
| 8638657 | 1128 | 66.CT.-G; 78.A.- | 1.311428923 | 0.33055537 |
| 6470437 | 1129 | 16.-.C; 86.-.G | 1.309929002 | 0.430012879 |
| 12102732 | 1130 | 2.A.-; 72.-.A | 1.307434337 | 0.918377829 |
| 8142718 | 1131 | 76.G.-; 129.C.A | 1.304595264 | 0.256619569 |
| 8156448 | 1132 | 77.-.C | 1.304175846 | 0.589870986 |
| 1852995 | 1133 | 0.TT.--; 75.-.C | 1.303475262 | 0.900561689 |
| 2887175 | 1134 | 1.-.C; 88.G.- | 1.302706726 | 0.597968881 |
| 2263396 | 1135 | 0.T.-; 85.T.- | 1.302466047 | 1.134047233 |
| 1825818 | 1136 | 0.TT.-A; 76.G.- | 1.301875777 | 1.110318533 |
| 8344169 | 1137 | 89.A.- | 1.301561654 | 1.225981484 |
| 2709285 | 1138 | 2.A.C; 0.T.-; | 1.30091689 | 0.894342408 |
| | | 82.-.C | | |
| 3023675 | 1139 | 1.TA.--; 82.A.-; | 1.299899754 | 0.818223111 |
| | | 84.A.T | | |
| 10084841 | 1140 | 19.-.T; 81.GA.-T | 1.297930762 | 0.600453513 |
| 1976248 | 1141 | 0.T.C; 86.-.C | 1.297836547 | 0.825789148 |
| 12154344 | 1142 | 2.A.-; 99.-.G | 1.296306945 | 1.001477179 |
| 13097626 | 1143 | -1.GT.--; 76.G.- | 1.295125439 | 0.441980787 |
| 6458438 | 1144 | 16.-.C; 76.-.A | 1.29467865 | 0.846781549 |
| 8150274 | 1145 | 77.-.A | 1.294485982 | 0.228877584 |
| 8757116 | 1146 | 55.-.T; 87.-.G | 1.292770836 | 0.600605612 |
| 2701481 | 1147 | 0.T.-; 2.A.C; | 1.291935395 | 0.554674604 |
| | | 87.C.T | | |
| 6458094 | 1148 | 16.-.C; 76.GG.-A | 1.289567023 | 1.072472271 |
| 8096141 | 1149 | 75.-.A; 87.-.G | 1.289021439 | 0.399874445 |
| 1937383 | 1150 | 0.TT.--; 2.A.C; | 1.288410807 | 1.057575643 |
| | | 76.GG.-C | | |
| 10527226 | 1151 | 15.-.T; 76.G.-; | 1.288081249 | 0.940790829 |
| | | 78.A.C | | |
| 2461285 | 1152 | 1.TA.--; 3.C.A | 1.288043851 | 1.103673268 |
| 9999142 | 1153 | 19.-.G; 73.A.- | 1.286125046 | 0.905401071 |
| 8190839 | 1154 | 85.TC.-- | 1.285570034 | 0.96890997 |
| 4021093 | 1155 | 3.-.C; 87.-.G | 1.285356603 | 0.94937054 |
| 8128562 | 1156 | 75.-.C; 132.G.C | 1.283817887 | 0.295940599 |
| 4026117 | 1157 | 3.-.C; 76.GG.-T | 1.282205843 | 0.870543947 |
| 3458694 | 1158 | 0.TTAC.----; | 1.2817117 | 1.235570501 |
| | | 75.-.C | | |
| 2402393 | 1159 | 1.-.A; 87.-.A | 1.281613783 | 0.828164871 |
| 1852100 | 1160 | 0.TT.--; 75.-.A | 1.281266877 | 0.682106006 |
| 3325688 | 1161 | 2.A.G; 0.T.-; | 1.280888677 | 0.892056905 |
| | | 78.A.- | | |
| 2742029 | 1162 | 0.T.-; 2.A.C; | 1.280778188 | 0.548022631 |
| | | 73.A.T | | |
| 6577492 | 1163 | 18.-.A; 86.-.C | 1.279802601 | 0.717533757 |
| 12218636 | 1164 | 2.A.-; 66.CT.-G | 1.279066994 | 0.773028062 |
| 8219007 | 1165 | 89.-.A | 1.278500325 | 1.111071537 |
| 6369323 | 1166 | 17.-.A; 76.GG.-T | 1.278457146 | 0.804381168 |
| 2651674 | 1167 | 0.T.-; 2.A.C; | 1.278172092 | 1.277273592 |
| | | 74.TC.-- | | |
| 12717259 | 1168 | 0.-.T; 74.-.C | 1.277376795 | 0.540831784 |
| 15160113 | 1169 | -29.A.G; | 1.277357928 | 0.269809108 |
| | | 76.GG.-A | | |
| 2900998 | 1170 | 1.-.C; 76.-.T | 1.277094929 | 0.459925786 |
| 1864123 | 1171 | 0.TT.--; 74.-.T | 1.275311167 | 0.782684718 |
| 1936243 | 1172 | 0.TT.--; 2.A.C; | 1.26922446 | 0.978313316 |
| | | 73.-.A | | |
| 10087310 | 1173 | 19.-.T; 76.-.G | 1.268648221 | 1.013020879 |
| 8128641 | 1174 | 131.A.C; 75.-.C | 1.268371306 | 0.347123635 |
| 2466267 | 1175 | 1.TA.--; 3.C.A; | 1.267812234 | 0.761193775 |
| | | 78.-.C | | |
| 14814370 | 1176 | -29.A.C; 74.-.T | 1.267572185 | 0.224895956 |
| 8367586 | 1177 | 86.-.G | 1.267571029 | 0.166811565 |
| 14814654 | 1178 | -29.A.C; | 1.267223704 | 0.299661636 |
| | | 75.CG.-T | | |
| 7178892 | 1179 | 27.-.A; 72.-.C | 1.266580365 | 1.241702285 |
| 2713900 | 1180 | 0.T.-; 2.A.C; | 1.266523416 | 1.064785518 |
| | | 82.AA.--; | | |
| | | 84.A.T | | |
| 12745658 | 1181 | 0.-.T; 78.A.- | 1.266094696 | 0.628742094 |
| 12436108 | 1182 | 1.TAC.---; 86.C.- | 1.265494144 | 0.683395752 |
| 8490474 | 1183 | 76.-.G; 131.A.C | 1.264843818 | 0.316333863 |
| 6479094 | 1184 | 16.-.C; 75.CG.-T | 1.264484483 | 0.657988122 |
| 10280354 | 1185 | 17.-.T; 75.-.A | 1.264238931 | 1.254859427 |
| 10528666 | 1186 | 15.-.T; 77.GA.-- | 1.264204883 | 1.069840201 |
| 10303386 | 1187 | 17.-.T; 82.AA.-- | 1.264094608 | 1.141678594 |
| 2355406 | 1188 | 0.T.-; 15.-.T | 1.26208998 | 0.699889425 |
| 3032160 | 1189 | 1.TA.--; 78.A.T | 1.261906598 | 0.661737928 |
| 7237755 | 1190 | 27.-.C; 72.-.C | 1.261808889 | 1.185044155 |
| 2295261 | 1191 | 0.T.-; 78.A.T | 1.261798645 | 0.619874643 |
| 14798078 | 1192 | -29.A.C; | 1.261281447 | 0.214857356 |
| | | 76.GG.-A | | |
| 3307911 | 1193 | 0.T.-; 2.A.G; | 1.259023231 | 0.786548058 |
| | | 86.-.G | | |
| 8132962 | 1194 | 75.-.C; 87.-.G | 1.259001218 | 0.463752754 |
| 10181383 | 1195 | 18.-.G; | 1.258323933 | 0.523286921 |
| | | 75.CG.-A | | |
| 8197001 | 1196 | 86.-.A | 1.256849633 | 0.486914942 |
| 10309927 | 1197 | 17.-.T; 76.G.-; | 1.256782087 | 0.744678415 |
| | | 78.A.T | | |
| 2301271 | 1198 | 0.T.-; 73.AT.-C | 1.256424659 | 0.81100738 |
| 13853791 | 1199 | -14.A.C; 75.-.G | 1.255450038 | 0.42561035 |
| 8538003 | 1200 | 75.-.G; 128.T.G | 1.255025364 | 0.362250327 |
| 8531397 | 1201 | 75.-.G; 88.G.- | 1.254071245 | 0.476939803 |
| 10088571 | 1202 | 19.-.T; 76.GG.-T | 1.253979064 | 0.431051128 |
| 10090672 | 1203 | 19.-.T; 74.-.T | 1.253721121 | 0.83319223 |
| 9978638 | 1204 | 19.-.G; 87.-.A | 1.253713731 | 0.820915459 |
| 10183679 | 1205 | 18.-.G; 76.G.-; | 1.253476631 | 0.445201573 |
| | | 78.A.C | | |
| 2283016 | 1206 | 0.T.-; 82.A.- | 1.252963004 | 0.465519392 |
| 2695201 | 1207 | 0.T.-; 2.A.C; | 1.25282914 | 0.803574579 |
| | | 91.A.G | | |
| 6475853 | 1208 | 16.-.C; 76.-.G | 1.250559059 | 0.663368638 |
| 6111106 | 1209 | 14.-.A; | 1.249881883 | 0.738247287 |
| | | 76.GG.-A | | |
| 3082312 | 1210 | 1.TA.--; 17.-.T | 1.249436868 | 0.812464001 |

TABLE 14

| | SEQ | | | |
|---|---|---|---|---|
| index | ID NO | muts_1indexed | MI | 95% CI |
| 10566255 | 1211 | 15.-.T; 73.AT.-C | 1.248872576 | 0.813225669 |
| 10070730 | 1212 | 19.-.T; 79.G.- | 1.248861015 | 0.601945811 |
| 14812876 | 1213 | -29.A.C; 76.GG.-T | 1.248067875 | 0.150831793 |
| 1246999 | 1214 | -15.T.G; 76.G.- | 1.247102347 | 0.224797578 |
| 8558498 | 1215 | 74.-.T; 132.G.C | 1.246022069 | 0.249030346 |
| 10518792 | 1216 | 15.-.T; 72.-.G | 1.245964164 | 0.488651001 |
| 4277925 | 1217 | 4.T.-; 84.AT.-- | 1.245854234 | 0.936943861 |
| 8352817 | 1218 | 86.C.- | 1.244532434 | 0.150629215 |
| 8538048 | 1219 | 75.-.G; 129.C.A | 1.244280774 | 0.412263647 |
| 14797557 | 1220 | -29.A.C; 75.-.A | 1.242782689 | 0.319674168 |
| 8538200 | 1221 | 75.-.G; 133.A.C | 1.241616447 | 0.440187544 |
| 4283490 | 1222 | 4.T.-; 82.-.C | 1.24156885 | 0.687466845 |
| 1865218 | 1223 | 0.TT.--; 73.A.- | 1.240690771 | 0.7042098 |
| 6525015 | 1224 | 17.-.G; 75.-.A | 1.240613105 | 0.979161775 |
| 10181717 | 1225 | 18.-.G; 76.GG.-A | 1.23997956 | 1.137575689 |
| 6458686 | 1226 | 16.-.C; 76.GG.-C | 1.239775702 | 0.87363525 |
| 9978404 | 1227 | 19.-.G; 86.-.A | 1.239174316 | 0.801664764 |
| 9631659 | 1228 | 16.------------. | 1.2381472 | 1.157545889 |
| | | CTCATTACTTTG | | |
| 1938525 | 1229 | 0.TT.--; 2.A.C; | 1.234976889 | 0.873037971 |
| | | 77.GA.-- | | |
| 1907202 | 1230 | 0.TTA.---; 3.C.A; | 1.234558517 | 0.900076058 |
| | | 87.-.G | | |
| 2315524 | 1231 | 0.T.-; 55.-.T | 1.234352592 | 0.65468754 |
| 8531688 | 1232 | 75.-.G; 89.-.A | 1.234168624 | 0.685214819 |
| 14798356 | 1233 | -29.A.C; 76.-.A | 1.233456387 | 0.88515606 |
| 8590491 | 1234 | 73.A.G | 1.232844488 | 0.306976558 |
| 3335980 | 1235 | 2.A.G; 0.T.-; 75.C.- | 1.23143562 | 0.615508551 |
| 2695420 | 1236 | 0.T.-; 2.A.C; | 1.23131981 | 1.032803346 |
| | | 91.AA.-G | | |
| 3307298 | 1237 | 0.T.-; 2.A.G; 87.-.T | 1.231275978 | 0.519311047 |
| 2560220 | 1238 | 0.T.-; 2.A.C; 14.-.A | 1.231165601 | 0.62236647 |
| 15165185 | 1239 | -29.A.G; 87.-.G | 1.231041719 | 0.270182884 |
| 12718005 | 1240 | 0.-.T; 74.-.G | 1.230670859 | 0.871174328 |
| 10058332 | 1241 | 19.-.T; 55.-.G | 1.229512018 | 1.083906642 |
| 8532180 | 1242 | 75.-.G; 98.-.A | 1.229364421 | 0.748719278 |
| 7242912 | 1243 | 27.-.C; 90.-.G | 1.229092331 | 0.949305592 |
| 8105731 | 1244 | 76.GG.-A; 131.A.C | 1.228181078 | 0.230343111 |
| 2748293 | 1245 | 2.A.C; 0.T.-; 66.C.- | 1.227763647 | 0.98496011 |
| 3026215 | 1246 | 1.TA.--; 77.GA.--; | 1.226977479 | 0.997524073 |
| | | 83.A.T | | |
| 1938157 | 1247 | 0.TT.--; 2.A.C; | 1.225574228 | 0.831200101 |
| | | 77.-.A | | |
| 11775381 | 1248 | 2.-.C; 76.G.- | 1.225102258 | 0.595949363 |
| 15161003 | 1249 | -29.A.G; 76.G.- | 1.223889061 | 0.294582862 |
| 14811016 | 1250 | -29.A.C; 78.-.C | 1.222938798 | 0.273221745 |
| 7237431 | 1251 | 27.-.C; 72.-.A | 1.221788719 | 1.142877721 |
| 4220887 | 1252 | 4.T.-; 72.-.C | 1.219780408 | 0.66608177 |
| 10561000 | 1253 | 15.-.T; 76.G.-; | 1.218871558 | 0.647994569 |
| | | 78.A.T | | |
| 3318946 | 1254 | 0.T.-; 2.A.G; | 1.217687896 | 0.704918875 |
| | | 81.GA.-T | | |
| 10565555 | 1255 | 15.-.T; 75.CG.-T | 1.217561106 | 1.206694498 |
| 2644619 | 1256 | 2.A.C; 0.T.-; | 1.217521416 | 0.643415599 |
| | | 72.-.C | | |
| 12112275 | 1257 | 2.A.-; 74.T.G | 1.217072779 | 0.652972838 |
| 1862409 | 1258 | 0.TT.--; 76.-.G | 1.217021239 | 0.888749766 |
| 7189944 | 1259 | 27.-.A; 78.-.T | 1.216123094 | 1.075111755 |
| 6126842 | 1260 | 14.-.A; 78.-.C | 1.215991705 | 0.768204394 |
| 8543659 | 1261 | 75.-.G; 88.-.G | 1.214712222 | 0.655007886 |
| 2684568 | 1262 | 2.A.C; 0.T.- | 1.213071327 | 0.264663522 |
| 2697264 | 1263 | 2.A.C; 0.T.-; | 1.2126732 | 1.021553423 |
| | | 89.A.G | | |
| 4285424 | 1264 | 4.T.-; 82.A.G | 1.211126496 | 1.094417444 |
| 4298510 | 1265 | 4T.-; 78.A.-; | 1.209030922 | 0.66844537 |
| | | 80.A.- | | |
| 3594929 | 1266 | 2.-.A; 87.-.T | 1.208764231 | 0.738646374 |
| 10310746 | 1267 | 17.-.T; 76.-.T | 1.208539188 | 0.919441484 |
| 6535421 | 1268 | 17.-.G; 74.-.T | 1.207908272 | 0.926692004 |
| 2738172 | 1269 | 0.T.-; 2.A.C73.-.G | 1.207771032 | 1.035065567 |
| 1942201 | 1270 | 0.TT.--; 2.A.C; | 1.207677897 | 0.973271683 |
| | | 87.-.G | | |
| 8518877 | 1271 | 76.GG.-T; | 1.206646593 | 0.182266975 |
| | | 121.C.A | | |
| 15159780 | 1272 | -29.A.G; 75.-.A | 1.205938094 | 0.315739517 |
| 2290805 | 1273 | 0.T.-; 79. | 1.204355839 | 0.868799816 |
| | | GAGAAA.TTTCTC | | |
| 2399086 | 1274 | 1.-.A; 76.GG.-A | 1.203971897 | 0.48437301 |
| 1974829 | 1275 | 0.T.C; 76.GG.-A | 1.203879032 | 0.4210079 |

TABLE 15

| | SEQ | | | |
|---|---|---|---|---|
| index | ID NO | muts_1indexed | MI | 95% CI |
| 1192019 | 1276 | -15.T.G; 0.T.-; | 1.20360799 | 0.302971783 |
| | | 2.A.C | | |
| 8565342 | 1277 | 75.CG.-T; 132.G.C | 1.202289742 | 0.286937554 |
| 8357813 | 1278 | 87.-.G; 132.G.C | 1.201504305 | 0.284156001 |
| 14647197 | 1279 | -29.A.C; 0.T.-; | 1.19977199 | 0.596254455 |
| | | 2.A.C; 75.-.G | | |
| 10192426 | 1280 | 18.-.G; 86.C.- | 1.197676147 | 0.845523053 |
| 2239077 | 1281 | 0.T.-; 65.GC.-A | 1.197039025 | 0.827792408 |
| 12185807 | 1282 | 2.A.-; 80.A.-82.A.- | 1.195795094 | 1.14774883 |
| 14921338 | 1283 | -29.A.C; 2.A.-; | 1.194753512 | 0.590835399 |
| | | 76.GG.-T | | |
| 1909484 | 1284 | 0.TTA.---; 3.C.A; | 1.194601681 | 0.899923073 |
| | | 74.-.T | | |
| 10067367 | 1285 | 19.-.T; 74.-.G | 1.194366583 | 0.703892606 |
| 8406855 | 1286 | 82.A.-; 84.A.T | 1.19422157 | 0.570093929 |
| 3084704 | 1287 | 1.TA.--; 15.-.T | 1.194024744 | 0.639373123 |
| 8117630 | 1288 | 76.GG.-C; 121.C.A | 1.193941022 | 0.493915898 |
| 14813162 | 1289 | -29.A.C; 76.-.T | 1.193770617 | 0.312340253 |
| 10086912 | 1290 | 19.-.T; 78.A.- | 1.193704359 | 0.526544832 |
| 8565389 | 1291 | 75.CG.-T; 132.G.T | 1.19331243 | 0.298806463 |
| 6627225 | 1292 | 18.C.-; 76.GG.-T | 1.192355135 | 0.550645762 |
| 8485326 | 1293 | 76.-.G; 86.-.C | 1.192298677 | 0.493607798 |
| 1853928 | 1294 | 0.TT.--; 79.G.- | 1.191920618 | 0.949329516 |
| 12437875 | 1295 | 1.TAC.---; 76.-.G | 1.191773341 | 0.823417938 |
| 10182569 | 1296 | 18.-.G; 75.-.C | 1.191543511 | 0.876936342 |
| 6584325 | 1297 | 18.-.A; 76.-.G | 1.190997627 | 0.955552088 |
| 8638758 | 1298 | 66.CT.-G; 76.-.G | 1.190381196 | 0.453916978 |
| 6460324 | 1299 | 16.-.C; 79.G.- | 1.190312109 | 0.493534915 |
| 8365015 | 1300 | 87.C.T | 1.190052456 | 0.872602313 |
| 8490408 | 1301 | 76.-.G | 1.18960287 | 0.31994112 |
| 6525955 | 1302 | 17.-.G; 75.-.C | 1.188288682 | 1.099927803 |
| 6460105 | 1303 | 16.-.C; 76.G.-; | 1.187507242 | 0.685448258 |
| | | 78.A.C | | |
| 6112043 | 1304 | 14.-.A; 75.-.C | 1.18750131 | 0.773401733 |
| 1978266 | 1305 | 0.T.C; 86.C.- | 1.186318648 | 0.482781507 |
| 8636881 | 1306 | 66.CT.-G; 87.-.G | 1.186183907 | 0.213972824 |
| 15241255 | 1307 | -29.A.G; 2.A.-; | 1.185988694 | 0.443745556 |
| | | 75.-.G | | |
| 6362433 | 1308 | 17.-.A; 76.GG.-A | 1.185910029 | 0.85106617 |
| 2059902 | 1309 | 0.TT.--; 2.A.G; | 1.185892464 | 1.168809929 |
| | | 74.-.T | | |
| 14799744 | 1310 | -29.A.C; 77.-.A | 1.185825684 | 0.192460709 |
| 8118273 | 1311 | 76.GG.-C; | 1.18519234 | 0.62982038 |
| | | 132.G.T | | |
| 4278865 | 1312 | 4.T.-; 84.-.T | 1.184410432 | 1.107710251 |
| 10065094 | 1313 | 19.-.T; 72.-.C | 1.1828142 | 0.675106042 |
| 8561350 | 1314 | 74.-.T; 87.-.G | 1.182048719 | 0.393482481 |
| 15160423 | 1315 | -29.A.G; | 1.180793171 | 0.555546714 |
| | | 76.GG.-C | | |
| 2994738 | 1316 | 1.TA.--; 74.T.G | 1.18058976 | 0.979631175 |
| 15058565 | 1317 | -29.A.G; 0.T.-; | 1.180163675 | 0.270139027 |
| | | 2.A.C | | |
| 12222182 | 1318 | 2.A.-; 65.GC.-T | 1.179771955 | 0.796494205 |
| 2881480 | 1319 | 1.-.C; 74.T.- | 1.179501503 | 0.538435597 |
| 10193035 | 1320 | 18.-.G86.-.G | 1.17845471 | 0.684536204 |
| 6459089 | 1321 | 16.-.C; 75.-.C | 1.17843793 | 0.58933484 |
| 10298749 | 1322 | 17.-.T; 89.-.C | 1.178374767 | 0.684239424 |
| 8490381 | 1323 | 76.-.G; 132.G.C | 1.177042107 | 0.335663686 |
| 12306660 | 1324 | 2.A.-; 18.-.G | 1.177019617 | 0.435298202 |
| 8124036 | 1325 | 75.-.C; 98.-.A | 1.176947131 | 0.49926186 |
| 2893687 | 1326 | 1.-.C; 88.-.T | 1.17496713 | 0.780013503 |
| 6305247 | 1327 | 16.-.A; 77.GA.-- | 1.174157138 | 0.633742635 |
| 7248579 | 1328 | 27.-.C; 83.-.T | 1.173562933 | 1.083697051 |
| 2883890 | 1329 | 1.-.C; 75.-.C | 1.173398841 | 0.613509504 |
| 10183041 | 1330 | 18.-.G; 76.G.- | 1.173134322 | 0.967093776 |
| 2696443 | 1331 | 0.T.-; 2.A.C; | 1.173067193 | 0.976987691 |
| | | 89.A.C | | |
| 15239681 | 1332 | -29.A.G; 2.A.-; | 1.173012223 | 0.486727112 |
| | | 76.G.- | | |
| 8087771 | 1333 | 74.-.G; 87.-.G | 1.172944262 | 0.426278168 |
| 10285497 | 1334 | 17.-.T; 79.G.- | 1.17154961 | 0.929605625 |
| 8118258 | 1335 | 76.GG.-C; | 1.170986028 | 0.499395392 |
| | | 133.A.C | | |
| 8141939 | 1336 | 76.G.-; 121.C.A | 1.17085979 | 0.256575176 |
| 8066677 | 1337 | 74.T.- | 1.168909113 | 0.239501292 |
| 8558553 | 1338 | 74.-.T; 132.G.T | 1.167854164 | 0.29356652 |
| 6469022 | 1339 | 16.-.C; 89.-.C | 1.167563507 | 0.467845833 |
| 1046356 | 1340 | -17.C.A; 75.-.G | 1.166966628 | 0.334507035 |
| 10532753 | 1341 | 15.-.T; 89.-.A | 1.16628898 | 0.941587373 |
| 2706855 | 1342 | 2.A.C; 0.T.-; | 1.165750392 | 0.619157804 |
| | | 83.-.G | | |
| 12194678 | 1343 | 2.A.-; 78.A.G | 1.165471135 | 0.91536488 |
| 12126149 | 1344 | 2.A.-; 77.-.C | 1.164066997 | 0.392106235 |
| 3039439 | 1345 | 1.TA.--; 70.-.T | 1.162844229 | 1.00756116 |
| 8123371 | 1346 | 75.-.C; 87.-.A | 1.161856358 | 0.505141299 |
| 15160286 | 1347 | -29.A.G; 76.-.A | 1.161712843 | 0.721602172 |
| 8758541 | 1348 | 55.-.T; 80.A.- | 1.160729144 | 0.587416563 |
| 12433294 | 1349 | 1.TAC.---; | 1.160546375 | 0.559999519 |
| | | 79.G.- | | |
| 14801714 | 1350 | -29.A.C87.-.A | 1.15970438 | 0.841171049 |
| 15058156 | 1351 | 2.A.C; 0.T.-; | 1.158508484 | 0.396829259 |
| | | -29.A.G; 76.G.- | | |
| 2298993 | 1352 | 0.T.-; 75.C.- | 1.158479025 | 0.419303739 |
| 13100965 | 1353 | -1.GT.--; 78.A.- | 1.158052786 | 0.371262978 |
| 8438445 | 1354 | 77.GA.--; 83.A.T | 1.156188842 | 0.838502061 |
| 8519469 | 1355 | 76.GG.-T; | 1.155859915 | 0.148192041 |
| | | 132.G.C | | |

TABLE 16

| | SEQ | | | |
|---|---|---|---|---|
| index | ID NO | muts_1indexed | MI | 95% CI |
| 8569101 | 1356 | 75.CGG.-TT | 1.154557321 | 0.217307834 |
| 4310993 | 1357 | 4.T.-;73.AT.-C | 1.153274081 | 0.453854703 |
| 9971050 | 1358 | 19.-.G;72.-.C | 1.152740318 | 0.725290861 |
| 2996647 | 1359 | 1.TA.--;75.CG.-A | 1.151902848 | 0.811777159 |
| 8561305 | 1360 | 74.-.T;86.C.- | 1.151372297 | 0.237653764 |
| 8093224 | 1361 | 75.-.A;129.C.A | 1.151362432 | 0.273047434 |
| 3323632 | 1362 | 2.A.G;0.T.-;78.AG.- | 1.150994398 | 0.848919541 |
| | | C | | |
| 14663326 | 1363 | - | 1.150191366 | 0.599920591 |
| | | 29.A.C;0.T.-;2.A.G; | | |
| | | 75.-.G | | |
| 1936729 | 1364 | 0.TT.- | 1.15004696 | 1.030340427 |
| | | -;2.A.C;74.-.G | | |
| 1977130 | 1365 | 0.T.C | 1.148209421 | 0.707223693 |
| 8141742 | 1366 | 120.C.A;76.G.- | 1.148153033 | 0.267222437 |
| 1908681 | 1367 | 0.TTA.-- | 1.14774524 | 0.964815 |
| | | -;3.C.A;76.-.G | | |
| 3017898 | 1368 | 1.TA.--;89.A.G | 1.147741635 | 0.737313223 |
| 3340495 | 1369 | 0.T.-;2.A.G;73.A.C | 1.147576225 | 1.09581674 |
| 2254255 | 1370 | 0.T.-;75.CG.-A | 1.146513584 | 0.700676298 |
| 11953402 | 1371 | 2.AC.- | 1.145157595 | 1.093445431 |
| | | -;4.T.C;76.GG.-C | | |
| 2684619 | 1372 | 0.T.-;2.A.C; 132.G.T | 1.144862088 | 0.260357332 |
| 10314306 | 1373 | 17.-.T;73.AT.-C | 1.144426663 | 1.028995367 |
| 10559572 | 1374 | 15.-.T;78.A.G | 1.143699755 | 0.578604678 |
| 2630318 | 1375 | 2.A.C;0.T.-;66.CT.- | 1.143660067 | 0.5343262 |
| | | A | | |
| 1943847 | 1376 | 0.TT.- | 1.142911019 | 0.764533182 |
| | | -;2.A.C;81.GA.-T | | |
| 4270685 | 1377 | 4.T.-;90.-.T | 1.142261105 | 1.061096734 |
| 8066737 | 1378 | 74.T.-;131.A.C | 1.142106376 | 0.297627826 |
| 6101577 | 1379 | 14.-.A;55.-.G | 1.141633238 | 0.632413834 |
| 4279604 | 1380 | 4.T.-;82.A.- | 1.141087787 | 0.86559009 |
| 2284176 | 1381 | 0.T.-;83.-.G | 1.140852012 | 0.573812016 |
| 6480468 | 1382 | 16.-.C;70.-.T | 1.1398625 | 0.613893735 |
| 2640116 | 1383 | 0.T.-;2.A.C;71.-.C | 1.13661499 | 0.936457355 |
| 10194587 | 1384 | 18.-.G;82.AA.-C | 1.136546503 | 0.867225106 |
| 15456465 | 1385 | -30.C.G;75.-.G | 1.136361233 | 0.420956305 |
| 3432602 | 1386 | 0.T.-;2.A.G;18.-.G | 1.136032616 | 0.358683183 |
| 8345813 | 1387 | 89.-.T | 1.134872739 | 0.634425715 |
| 3023247 | 1388 | 1.TA.--;83.-.T | 1.134857334 | 0.960489164 |
| 10472698 | 1389 | 16.C.-;76.-.G | 1.134422965 | 0.910950327 |
| 1855129 | 1390 | 0.TT.--;88.G.- | 1.133496442 | 0.758584634 |
| 9993029 | 1391 | 19.-.G;78.A.- | 1.133174297 | 0.792593276 |
| 15168776 | 1392 | -29.A.G;76.GG.-T | 1.132498922 | 0.227015084 |
| 2464359 | 1393 | 1.TA.- | 1.131831655 | 1.057358093 |
| | | -;3.C.A;82.A.-;84.A. | | |
| | | G | | |
| 12156161 | 1394 | 2.A.-;98.-.T | 1.130993969 | 0.851874656 |
| 8544614 | 1395 | 75.-.G;82.A.- | 1.130902206 | 0.457628408 |
| 2278784 | 1396 | 0.T.-;89.A.G | 1.129976098 | 0.932328577 |
| 4229697 | 1397 | 4.T.-;75.CG.-A | 1.129356919 | 1.031398221 |
| 6461360 | 1398 | 16.-.C;82.-.A | 1.129237794 | 0.60908879 |
| 8128601 | 1399 | 133.A.C;75.-.0 | 1.129022276 | 0.316118395 |
| 6362009 | 1400 | 17.-.A;74.-.G | 1.127775382 | 0.792324832 |
| 14806733 | 1401 | -29.A.C;86.C.- | 1.127749344 | 0.128149617 |
| 1937160 | 1402 | 0.TT.- | 1.126385937 | 0.99995983 |
| | | -;2.A.C;76.GG.-A | | |
| 4311644 | 1403 | 4.T.-;73.A.C | 1.126234133 | 0.593451059 |
| 1863149 | 1404 | 0.TT.--;76.GG.-T | 1.126088195 | 0.642579265 |
| 15169751 | 1405 | -29.A.G;74.-.T | 1.12571698 | 0.264785044 |
| 14811726 | 1406 | -29.A.C;76.-.G | 1.125696747 | 0.337727802 |
| 6480066 | 1407 | 16.-.C;73.AT.-G | 1.125267029 | 0.917637118 |
| 3014440 | 1408 | 1.TA.--;98.-.T | 1.125187087 | 0.944870769 |
| 6473404 | 1409 | 16.-.C;82.AA.-T | 1.125183194 | 0.45047498 |
| 7179375 | 1410 | 27.-.A;73.-.A | 1.12275521 | 1.11852897 |
| 12303885 | 1411 | 2.A.-;19.-.T | 1.122538412 | 0.456330423 |
| 2267762 | 1412 | 0.T.-;98.-.A | 1.122023688 | 0.678726891 |
| 10318319 | 1413 | 17.-.T;66.CT.-G | 1.121565522 | 1.049618975 |
| 8093357 | 1414 | 75.-.A;132.G.T | 1.121299918 | 0.315044761 |
| 3027775 | 1415 | 1.TA.--;80.AG.-T | 1.120820262 | 0.672573613 |
| 10549691 | 1416 | 15.-.T;82.A.- | 1.11965366 | 0.843624461 |
| 8558571 | 1417 | 74.-.T;131.A.C | 1.119006524 | 0.242404014 |
| 12210725 | 1418 | 2.A.-;73.AT.-G | 1.118721361 | 0.804765677 |
| 6462677 | 1419 | 16.-.C;86.-.0 | 1.118051706 | 0.993606042 |
| 2281811 | 1420 | 0.T.-;86.CC.-T | 1.117740311 | 0.882847082 |
| 8496336 | 1421 | 78.A.-;80.A.- | 1.11711092 | 0.515102154 |
| 3038148 | 1422 | 1.TA.--;73.A.0 | 1.116865927 | 0.861601124 |
| 10199335 | 1423 | 75.-.G;127.T.G | 1.115860528 | 0.443672147 |
| 14801930 | 1424 | -29.A .C;88.G.- | 1.115492358 | 0.261525199 |
| 2885740 | 1425 | 1.-.C;81.GA.-C | 1.115472314 | 0.689247174 |
| 8436871 | 1426 | 81.GA.-T | 1.115411316 | 0.273931065 |
| 6533591 | 1427 | 17.-.G;78.-.C | 1.115398223 | 0.879526979 |
| 8508461 | 1428 | 78.A.T | 1.115273341 | 0.522766505 |
| 2303258 | 1429 | 0.T.-;70.-.T | 1.114089034 | 0.865293893 |
| 10200479 | 1430 | 18.-.G;75.CG.-T | 1.11302882 | 0.732217972 |
| 8142460 | 1431 | 76.G.-;126.C.A | 1.111268298 | 0.288237659 |
| 8490449 | 1432 | 76.-.G;132.G.T | 1.111184304 | 0.315337948 |
| 1862090 | 1433 | 0.TT.--;78.A.- | 1.110821771 | 0.799594856 |
| 8105143 | 1434 | 76.GG.-A;121.C.A | 1.110817347 | 0.256306387 |
| 10204124 | 1435 | 18.-.G;65.GC.-T | 1.110123297 | 0.661140904 |
| 2696979 | 1436 | 0.T.-2.A.C;88.-.G | 1.109825686 | 0.606525063 |
| 1246393 | 1437 | -15.T.G;76.GG.-A | 1.109540149 | 0.193534821 |
| 4277641 | 1438 | 4.T.-;84.-.C | 1.109476081 | 1.084635844 |
| 12163684 | 1439 | 2.A.-;88.-.G | 1.108884791 | 0.569947232 |
| 3643882 | 1440 | 3.CT.-A;76.GG.-A | 1.108525297 | 0.784501998 |
| 6461122 | 1441 | 16.-.C;81.GA.-C | 1.108411865 | 0.6256586 |
| 14645694 | 1442 | 2.A.C;0.T.-;-29.A.C | 1.108180575 | 0.267740202 |
| 2678659 | 1443 | 0.T.-;2.A.C;98.-.A | 1.108043817 | 0.375625961 |
| 2295085 | 1444 | 0.T.-;77.GA.- | 1.107908285 | 0.695122129 |
| | | -;80.A.T | | |
| 8127785 | 1445 | 75.-.C; 120.C.A | 1.107076026 | 0.298513014 |
| 8357871 | 1446 | 87.-.G;132.G.T | 1.106990466 | 0.336105007 |
| 12090020 | 1447 | 2.A.-;66.CT.-A | 1.106107395 | 0.759889566 |
| 3079463 | 1448 | 1.TA.--;19.-.T | 1.105122706 | 0.424402722 |
| 10277558 | 1449 | 17.-.T;72.-.G | 1.105013965 | 0.33485503 |
| 2694724 | 1450 | 0.T.-;2.A.C;92.A.T | 1.102493901 | 0.92875617 |
| 3135565 | 1451 | 1.T.G;3.C.-;75.C.- | 1.102427225 | 0.672977559 |
| 6304328 | 1452 | 16.-.A;75.-.0 | 1.102231603 | 0.655223933 |
| 2708067 | 1453 | 2.A.C;0.T.-;83.-.T | 1.102074657 | 0.85908326 |

TABLE 17

| | SEQ | | | |
|---|---|---|---|---|
| index | ID NO | muts_1indexed | MI | 95% CI |
| 6469331 | 1454 | 16.-.C;89.A.- | 1.101247124 | 0.790943347 |
| 10073526 | 1455 | 19.-.T;90.T.- | 1.100917015 | 0.917104807 |
| 3017595 | 1456 | 1.TA.--;89.AT.-G | 1.100705976 | 0.903502652 |
| 3031194 | 1457 | 1.TA.--;78.A.G | 1.100353042 | 1.041515667 |
| 12123777 | 1458 | 2.A.-;76.G.-;132.G.C | 1.099950644 | 0.426062735 |
| 15451300 | 1459 | -30.C.G;76.G.- | 1.099949995 | 0.258120629 |
| 8105041 | 1460 | 76.GG.-A;120.C.A | 1.099511776 | 0.197987545 |
| 2894267 | 1461 | 1.-.C;87.-.T | 1.099423144 | 0.721770941 |
| 2998547 | 1462 | 1.TA.--;76.GG.-C | 1.099108914 | 0.77205836 |
| 3022051 | 1463 | 1.TA.--;83.-.C | 1.098959048 | 0.800244551 |
| 8512487 | 1464 | 76.G.-;78.A.T | 1.098356606 | 0.434447312 |
| 2285757 | 1465 | 0.T.-;82.AA.-C | 1.09769235 | 0.581396293 |
| 6531470 | 1466 | 17.-.G;87.-.G | 1.097040084 | 0.891732461 |
| 3461447 | 1467 | 0.TTAC.----;78.A.- | 1.096939612 | 1.032099163 |
| 6475031 | 1468 | 16.-.C;78.-.C | 1.096131509 | 0.622829146 |
| 10194914 | 1469 | 18.-.G;82.AA.-G | 1.095184273 | 0.925851293 |
| 1041972 | 1470 | -17.C.A;76.G.- | 1.094390364 | 0.259851818 |
| 8537811 | 1471 | 75.-.G;126.C.A | 1.093652258 | 0.416192839 |
| 3020817 | 1472 | 1.TA.--;84.AT.-- | 1.093578537 | 1.006083902 |
| 2887379 | 1473 | 1.-.C;86.-.C | 1.09339523 | 0.649567308 |
| 1854285 | 1474 | 0.TT.--;77.GA.-- | 1.093372662 | 0.836050071 |
| 8357326 | 1475 | 87.-.G;121.C.A | 1.09282229 | 0.228022974 |
| 8128534 | 1476 | 75.-.C;130.T.G | 1.091710468 | 0.291584852 |
| 1947291 | 1477 | 0.TT.--;2.A.C;73.A.- | 1.091598518 | 1.082985081 |
| 12432721 | 1478 | 1.TAC.---;76.GG.-C | 1.091484949 | 0.424680956 |
| 1252779 | 1479 | -15.T.G;75.-.G | 1.091018899 | 0.435778338 |
| 3588353 | 1480 | 2.-.A;86.-.0 | 1.090352944 | 0.473490794 |
| 2900664 | 1481 | 1 .-.C;76.GG.-T | 1.090288414 | 0.927626492 |
| 8076983 | 1482 | 74.T.G | 1.090265095 | 0.516206235 |
| 2300899 | 1483 | 0.T.-;73.-.C | 1.088155007 | 0.922134256 |
| 12202788 | 1484 | 2.A.-;75.-.G;132.G.C | 1.086592764 | 0.396856807 |
| 10070325 | 1485 | 19.-.T;77.-.A | 1.085159477 | 0.602291028 |
| 14685826 | 1486 | -29.A.C;4.T.-;76.G.- | 1.084700709 | 0.875467461 |
| 14351033 | 1487 | -25.A.C;75.-.G | 1.084694375 | 0.401588153 |
| 8607376 | 1488 | 73.A.T | 1.084223593 | 0.466050446 |
| 12439360 | 1489 | 1.TAC.---;73.A.- | 1.08377761 | 0.784604612 |
| 12718596 | 1490 | 0.-.T;75.-.A | 1.082686019 | 0.729622493 |
| 2712801 | 1491 | 2.A.C;0.T.-;82.A.T | 1.082648143 | 1.029910332 |
| 6613293 | 1492 | 18.C.-;77.-.C | 1.081600577 | 0.704127135 |
| 8480766 | 1493 | 78.A.- | 1.080656792 | 0.244162899 |
| 2414074 | 1494 | 1.-.A;75.CG.-T | 1.078260507 | 0.690226021 |
| 8105662 | 1495 | 76.GG.-A;132.G.C | 1.078192392 | 0.265594919 |
| 2282078 | 1496 | 0.T.-;84.AT.-- | 1.077981676 | 1.017841506 |
| 8096091 | 1497 | 75.-.A;86.C.- | 1.077805608 | 0.284536894 |
| 442111 | 1498 | -27.C.A;76.GG.-C | 1.077745882 | 0.495264554 |
| 12161656 | 1499 | 2.A.-;91.A.G | 1.075879018 | 0.678047969 |
| 9997135 | 1500 | 19.-.G;75.CG.-T | 1.075769653 | 0.617579849 |
| 6480747 | 1501 | 16.-.C;73.A.- | 1.074075162 | 0.613495205 |
| 8066659 | 1502 | 74.T.-;132.G.C | 1.073725216 | 0.262916351 |
| 4265165 | 1503 | 4.T.-;99.-.G | 1.07334647 | 0.742133576 |
| 8212888 | 1504 | 86.-.C;132.G.T | 1.071784689 | 0.489573855 |
| 10532402 | 1505 | 15.-.T;88.GA.-C | 1.071101998 | 0.564708496 |
| 2897244 | 1506 | 1.-.C;81.GA.-T | 1.07106925 | 0.381005159 |
| 2274809 | 1507 | 0.T.-;98.-.T | 1.071006931 | 0.70160388 |
| 3584484 | 1508 | 2.-.A;76.GG.-C | 1.070634794 | 0.859304506 |
| 12115802 | 1509 | 2.A.-;75.CG.-A | 1.070285621 | 0.735963692 |
| 3349186 | 1510 | 2.A.G;0.T.-;66.CT.-G | 1.06950253 | 0.942756466 |
| 3314448 | 1511 | 0.T.-;2.A.G;82.A.-84. | 1.069109584 | 0.669577854 |
| | | A.T | | |
| 2882882 | 1512 | 1.-.C;76.GG.-A | 1.068897247 | 0.641235084 |
| 8112365 | 1513 | 132.G.C;76.-.A | 1.068484818 | 0.642427564 |
| 8118289 | 1514 | 76.GG.-C;131.A.C | 1.067607855 | 0.671530402 |
| 2684538 | 1515 | 0.T.-2.A.C132.G.C | 1.067511236 | 0.29169754 |
| 3305808 | 1516 | 2.A.G;0.T.-;86.C.- | 1.067367495 | 0.81480322 |
| 12141962 | 1517 | 2.A.-;98.-.A | 1.06684638 | 0.768887059 |
| 8629287 | 1518 | 66.CT.-G;87.-.A | 1.066757603 | 0.520708474 |
| 10548927 | 1519 | 15.-.T;84.-.G | 1.066135811 | 0.948733575 |
| 12437589 | 1520 | 1.TAC.---;78.-.C. | 1.066060316 | 1.009600092 |
| 8494451 | 1521 | 76.-.G;87.-.G | 1.065178507 | 0.356343345 |
| 8148054 | 1522 | 76.G.-;87.-.G | 1.064941808 | 0.413919716 |
| 2684598 | 1523 | 0.T.-;2.A.C;133.A.C | 1.064210221 | 0.264316583 |
| 1806606 | 1524 | -3.TAGT.----;76.G.- | 1.063373097 | 0.955312128 |
| 6112609 | 1525 | 14.-.A;76.G.- | 1.062684812 | 0.689632914 |
| 8128619 | 1526 | 75.-.C;132.G.T | 1.062529409 | 0.341411659 |
| 2263869 | 1527 | 0.T.-;85.-.G | 1.062153729 | 1.016617311 |
| 8519538 | 1528 | 76.GG.-T;131.A.C | 1.061496162 | 0.210300359 |
| 15167837 | 1529 | -29.A.G;78.A.- | 1.061156026 | 0.246892291 |
| 8539891 | 1530 | 113.A.C;75.-.G | 1.061040443 | 0.379626895 |
| 6110621 | 1531 | 14.-.A;75.-.A | 1.060284727 | 0.621027153 |
| 4012102 | 1532 | 3.-.C;76.GG.-A | 1.059255634 | 1.031842175 |
| 14644765 | 1533 | - | 1.058597553 | 0.329942143 |
| | | 29.A.C;0.T.-;2.A.C;76 | | |
| | | .GG.-A | | |
| 6114928 | 1534 | 14.-.A;87.-.A | 1.058454656 | 0.885887929 |
| 1858781 | 1535 | 0.TT.--;87.-.T | 1.058406061 | 0.825333202 |
| 10090936 | 1536 | 19.-.T;75.CG.-T | 1.055554876 | 0.65945615 |
| 2002673 | 1537 | 0.TTA.---;86.-.C | 1.055214988 | 0.912819901 |
| 1937274 | 1538 | 0.TT.--;2.A.C;76.-.A | 1.054745159 | 0.766113106 |
| 1946930 | 1539 | 2.A.C;0.TT.--;73.AT.- | 1.053796386 | 1.042376689 |
| | | G | | |
| 8564806 | 1540 | 75.CG.-T;121.C.A | 1.053601658 | 0.274429264 |
| 14646874 | 1541 | - | 1.053406381 | 0.59545095 |
| | | 29.A.C;0.T.-;2.A.C78 | | |
| | | .A.- | | |
| 3279449 | 1542 | 2.A.G;0.T.-;86.-.A | 1.052984275 | 0.589481391 |
| 10183929 | 1543 | 18.-.G;79.G.- | 1.052474243 | 0.657984499 |
| 4281239 | 1544 | 4.T.-;83.-.G | 1.052428885 | 0.86399563 |
| 8636987 | 1545 | 66.CT.-G;87.-.T | 1.051957568 | 0.462896567 |
| 2684414 | 1546 | 129.C.A;2.A.C;0.T.- | 1.050747476 | 0.311891892 |
| 10567800 | 1547 | 15.-.T;70.-.T | 1.050309671 | 0.621437389 |
| 12183487 | 1548 | 2.A.-;77.GA.--;83.A.T | 1.049084957 | 0.987091579 |
| 3429655 | 1549 | 0.T.-;2.A.G;19.-.T | 1.048854899 | 0.495285429 |
| 15168064 | 1550 | -29.A.G;76.-.G | 1.047823892 | 0.302363264 |
| 8579268 | 1551 | 73.A.C | 1.047594299 | 0.683277383 |
| 12725378 | 1552 | 0.-.T;86.-.A | 1.047411001 | 0.365860881 |
| 12133179 | 1553 | 2.A.-;85.TC.-- | 1.046943252 | 0.820385361 |
| 12169171 | 1554 | 2.A.-;87.C.T | 1.046922375 | 0.599814315 |
| 1974530 | 1555 | 0.T.C;74.-.G | 1.045406007 | 0.681746678 |
| 3276852 | 1556 | 2.A.G;0.T.-;81.GA.-C | 1.045355433 | 0.975208443 |
| 2277126 | 1557 | 0.T.-;91.A.-;93.A.G | 1.044132704 | 0.955042692 |
| 2668148 | 1558 | 0.T.-;2.A.C;80.-.A | 1.043324984 | 0.586273368 |
| 1946365 | 1559 | 0.TT.--;2.A.C;74.-.T | 1.042813973 | 1.040869889 |
| 10086224 | 1560 | 19.-.T;78.AG.-C | 1.042716835 | 0.735960104 |
| 6474902 | 1561 | 16.-.C;78.AG.-C | 1.042498444 | 0.502799595 |
| 3001790 | 1562 | 1.TA.--;77.-.C | 1.042102465 | 0.683500309 |
| 6463023 | 1563 | 16.-.C;89.-.A | 1.041885948 | 0.829735162 |
| 8470293 | 1564 | 78.-.C;132.G.T | 1.041802211 | 0.300184554 |
| 3134206 | 1565 | 1.T.G;3.C.- | 1.041152356 | 0.79291182 |
| 10203551 | 1566 | 18.-.G;66.CT.-G | 1.039956878 | 0.786827483 |
| 8629503 | 1567 | 66.CT.-G;86.-.C | 1.039159805 | 0.369657454 |
| 13846013 | 1568 | -14.A.C;76.G.- | 1.038294775 | 0.247154929 |
| 2263715 | 1569 | 0.T.-;85.TC.-G | 1.038283386 | 0.801663086 |
| 10560681 | 1570 | 15.-.T;78.A.T | 1.037822098 | 0.677021869 |
| 1253221 | 1571 | -15.T.G;75.CG.-T | 1.037675362 | 0.212533654 |
| 10556907 | 1572 | 15.-.T;78.AG.-C | 1.037273554 | 1.01979448 |
| 3319204 | 1573 | 0.T.-;2.A.G;77.GA.- | 1.035671503 | 0.978042547 |
| | | -;83.A.T | | |
| 2277677 | 1574 | 0.T.-;91.AA.-G | 1.035145434 | 0.944699856 |
| 3044097 | 1575 | 1.TA.--;65.GC.-T | 1.033908393 | 0.776681137 |
| 2728986 | 1576 | 0.T.-;2.A.C76.GG.- | 1.033146947 | 0.961151984 |
| | | -;78.A.T | | |
| 15059527 | 1577 | - | 1.032618019 | 0.530633171 |
| | | 29.A.G;0.T.-;2.A.C;75 | | |
| | | .-.G | | |
| 8127925 | 1578 | 75.-.C121.C.A | 1.031822771 | 0.245553704 |
| 8069875 | 1579 | 74.T.-;87.-.G | 1.031655887 | 0.582873666 |
| 4210905 | 1580 | 4.T.-;66.CT.-A | 1.031653511 | 0.842224225 |
| 393375 | 1581 | -27.C.A;0.T.-;2.A.C | 1.031022939 | 0.248514229 |
| 6469193 | 1582 | 16.-.C;88.-.G | 1.030464034 | 0.735892666 |
| 12723788 | 1583 | 0.-.T;77.GA.-- | 1.02991096 | 0.435853484 |
| 1975104 | 1584 | 0.T.C;75.-.C | 1.029831571 | 0.578621416 |
| 447486 | 1585 | -27.C.A;74.-.T | 1.029567827 | 0.222259337 |
| 2304326 | 1586 | 0.T.-;73.A.T | 1.028839146 | 0.531317588 |
| 8480805 | 1587 | 78.A.-;132.G.T | 1.028699655 | 0.24544604 |
| 10289207 | 1588 | 17.-.T;89.-.A | 1.026291461 | 0.760292997 |
| 10541758 | 1589 | 15.-.T;99.-.G | 1.025988854 | 0.736311706 |
| 8580639 | 1590 | 73.-TC.G-- | 1.025947068 | 0.358873945 |
| 2129400 | 1591 | 0.TTA.-- | 1.025918395 | 1.011043018 |
| | | -;3.C.G.74.-.T | | |
| 8142671 | 1592 | 76.G.-;128.T.G | 1.025910634 | 0.290060081 |
| 12726231 | 1593 | 0.-.T;88.G.- | 1.025634121 | 0.405083637 |
| 10288957 | 1594 | 17.-.T;88.GA.-C | 1.025294913 | 0.60244436 |
| 2982939 | 1595 | 1.TA.--;65.GC.-A | 1.024519789 | 0.854258194 |
| 8357852 | 1596 | 87.-.G;133.A.C | 1.024422549 | 0.266728008 |
| 6626305 | 1597 | 18.C.-;76.-.G | 1.023762958 | 0.940900038 |
| 15167605 | 1598 | -29.A.G;78.-.C | 1.023529076 | 0.227603078 |
| 3273923 | 1599 | 2.A.G;0.T.-;79.G.- | 1.021930112 | 0.761031763 |
| 10553626 | 1600 | 15.-.T;82.AA.-T | 1.019809642 | 0.843756794 |
| 3029129 | 1601 | 1.TA.--;78.A.C | 1.018314726 | 0.493342655 |
| 3133667 | 1602 | 1.T.G;3.C.-;76.G.- | 1.018063645 | 0.663755989 |
| 14921066 | 1603 | -29.A.C;2.A.-;78.A.- | 1.01768547 | 0.653829676 |
| 14806598 | 1604 | -29.A.C;88.-.T | 1.01731078 | 0.326928264 |
| 8139512 | 1605 | 115.T.G;76.G.- | 1.017267726 | 0.260385137 |
| 8636794 | 1606 | 66.CT.-G;86.C.- | 1.016727519 | 0.223982922 |
| 8127584 | 1607 | 75.-.C;119.C.A | 1.016622667 | 0.257590784 |
| 4311933 | 1608 | 4.T.-;73.-.G | 1.015685468 | 0.722112585 |
| 6471359 | 1609 | 16.-.C;83.-.C | 1.01562419 | 0.689800797 |
| 12433542 | 1610 | 1.TAC.---;77.GA.-- | 1.015490193 | 0.963013214 |
| 8093303 | 1611 | 75.-.A;132.G.C | 1.014481628 | 0.287331894 |
| 1246761 | 1612 | -15.T.G;75.-.C | 1.013809204 | 0.244509289 |
| 1943763 | 1613 | 0.TT.--;2.A.C;82.AA.- | 1.01333782 | 0.875914657 |
| | | T | | |
| 4158980 | 1614 | 4.T.-;16.-.C | 1.012370327 | 0.730848589 |
| 8470306 | 1615 | 78.-.C;131.A.C | 1.011978039 | 0.268703426 |
| 8069089 | 1616 | 74.T.-;98.-.T | 1.011870417 | 0.753778629 |
| 12438882 | 1617 | 1.TAC.---;75.CG.-T | 1.011591105 | 0.646464747 |
| 8338521 | 1618 | 89.AT.-G | 1.01013237 | 0.921901816 |
| 10088951 | 1619 | 19.-.T;76.-.T | 1.009998244 | 0.995271538 |
| 12163085 | 1620 | 2.A.-;89.A.C | 1.009951212 | 1.005859847 |
| 8479927 | 1621 | 78.A.-;121.C.A | 1.007731759 | 0.198019758 |
| 10196772 | 1622 | 18.-.G;78.A.C | 1.007451686 | 0.605771645 |
| 8552295 | 1623 | 75.C.-;87.-.G | 1.006469896 | 0.446050968 |
| 4027916 | 1624 | 3.-.C;74.-.T | 1.006243971 | 0.88765081 |
| 8489338 | 1625 | 76.-.G;119.C.A | 1.005065199 | 0.338308183 |
| 446968 | 1626 | -27.C.A;76.GG.-T | 1.005048486 | 0.187310862 |
| 2049927 | 1627 | 0.TT.--;2.A.G;88.G.- | 1.004518203 | 0.953193053 |
| 8598621 | 1628 | 70.-.T;87.-.G | 1.004188688 | 0.382729413 |
| 8600573 | 1629 | 73.A.-;86.-.C | 1.004072362 | 0.368500944 |
| 8473900 | 1630 | 78.A.C | 1.003342068 | 0.272291839 |
| 12174360 | 1631 | 2.A.-;83.-.C | 1.002121947 | 0.61218072 |
| 442458 | 1632 | -27.C.A;76.G.- | 1.000814752 | 0.255096372 |
| 15162537 | 1633 | -29.A.G;86.-.C | 0.999559775 | 0.511729714 |
| 2991036 | 1634 | 1.TA.--;72.-.C | 0.998951084 | 0.524247852 |
| 8489557 | 1635 | 76.-.G;120.C.A | 0.998819409 | 0.234587818 |
| 2704195 | 1636 | 0.T.-;2.A.C;84.A.G | 0.998758579 | 0.779291093 |
| 12746931 | 1637 | 0.-.T;78.AG.-T | 0.998623067 | 0.694500161 |
| 8544289 | 1638 | 75.-.G;86.-.G | 0.998103804 | 0.329574932 |
| 8490052 | 1639 | 76.-.G;126.C.A | 0.998093656 | 0.284212266 |
| 3003857 | 1640 | 1.TA.--;81.GA.-C | 0.997215707 | 0.622492253 |
| 2683589 | 1641 | 0.T.-;2.A.C;121.C.A | 0.996781493 | 0.258997418 |
| 8565256 | 1642 | 75.CG.-T;129.C.A | 0.995682253 | 0.263828668 |
| 2684649 | 1643 | 0.T.-;2.A.C;131.A.C | 0.99524259 | 0.271694246 |
| 10192242 | 1644 | 18.-.G88.-.T | 0.995235176 | 0.989010874 |
| 8128468 | 1645 | 75.-.C;129.C.A | 0.994697493 | 0.26199099 |
| 3255338 | 1646 | 2.A.G;0.T.-;72.-.C | 0.994393387 | 0.842137355 |
| 7829410 | 1647 | 55.-.G;75.-.C | 0.994082042 | 0.859909204 |
| 15162331 | 1648 | -29.A.G;87,-.A | 0.993077228 | 0.690696181 |
| 8212834 | 1649 | 86.-.C;132.G.C | 0.991782036 | 0.466773251 |
| 13222300 | 1650 | 2.A.G;-3.TAGT.--- | 0.991302063 | 0.722815444 |
| | | -;76.G.- | | |
| 8470255 | 1651 | 78.-.C;132.G.C | 0.990938343 | 0.219379454 |
| 2661937 | 1652 | 132.G.C;2.A.C;0.T.-;7 | 0.989945596 | 0.389653762 |
| | | 6.G.- | | |
| 2670761 | 1653 | 0.T.-;2.A.C;85.TCC.-- | 0.989731739 | 0.7195275 |
| | | - | | |
| 11776916 | 1654 | 2.-.C;87.-.A | 0.989233941 | 0.938218378 |
| 12747759 | 1655 | 0.-.T;77.-.T | 0.989194317 | 0.937953146 |
| 15165085 | 1656 | -29.A.G;86.C.- | 0.987044987 | 0.176311237 |
| 8212745 | 1657 | 86.-.C;129.C.A | 0.987010247 | 0.50896412 |
| 2989789 | 1658 | 1.TA.--;72.-.A | 0.986062777 | 0.659043613 |
| 6531564 | 1659 | 17.-.G;87.-.T | 0.985471522 | 0.962121285 |
| 12436169 | 1660 | 1.TAC.---;87.-.G | 0.984379414 | 0.678230211 |
| 3311127 | 1661 | 2.A.G;0.T.-;82.A.- | 0.983849984 | 0.759053343 |
| 2264270 | 1662 | 0.T.-;86.CC.-A | 0.983283085 | 0.774791896 |
| 10091719 | 1663 | 19.-.T;73.AT.-G | 0.982030918 | 0.402281056 |
| 8143233 | 1664 | 76.G.-;123.A.C | 0.98195845 | 0.225973301 |
| 1248077 | 1665 | -15.T.G;86.-.C | 0.981472735 | 0.61947878 |

TABLE 18

| | SEQ | | | |
|---|---|---|---|---|
| index | ID NO | muts_1indexed | MI | 95% CI |
| 12716866 | 1666 | 0.-.T;74.T.- | 0.980705762 | 0.501255257 |
| 3303133 | 1667 | 2.A.G;0.T.-;89.-.C | 0.980281754 | 0.929335139 |
| 9974910 | 1668 | 19.-.G;76.GG.-C | 0.980161229 | 0.702243506 |
| 8143415 | 1669 | 76.G.-;122.A.C | 0.979878321 | 0.246975709 |
| 1981670 | 1670 | 0.T.C;74.-.T | 0.979604036 | 0.59020272 |
| 2302384 | 1671 | 0.T.-;73.AT.-G | 0.978319856 | 0.564838423 |
| 1809039 | 1672 | -3.TAGT.----;78.A.- | 0.978230395 | 0.8011754 |
| 13139359 | 1673 | -I .G.-;2.A.C | 0.97786126 | 0.274956142 |
| 8538659 | 1674 | 75.-.G;122.A.C | 0.977608955 | 0.391570629 |
| 2651461 | 1675 | 0.T.-;2.A.C;74.T.G | 0.976860498 | 0.581709587 |
| 3028256 | 1676 | 1.TA.--;79.GA.-T | 0.976555598 | 0.767447405 |
| 444970 | 1677 | -27.C.A;87.-.G | 0.976499126 | 0.225151793 |
| 2271218 | 1678 | 132.G.T;0.T.- | 0.976357981 | 0.375657527 |
| 13101059 | 1679 | -1.GT.--;76.-.G | 0.97610403 | 0.319731571 |
| 15169928 | 1680 | -29.A.G;75.CG.-T | 0.976070783 | 0.275722437 |
| 6454149 | 1681 | 16.-.C;72.-.C | 0.975765291 | 0.471747331 |
| 8519506 | 1682 | 76.GG.-T;133.A.C | 0.975539914 | 0.183246169 |
| 1936400 | 1683 | 0.TT.--;2.A.C;74.T.- | 0.974896363 | 0.971225863 |
| 8363289 | 1684 | 87.-.T;132.G.T | 0.974823104 | 0.348800323 |
| 14646928 | 1685 | - | 0.974746731 | 0.273309529 |
| | | 29.A.C;0.T.-;2.A.C;76 | | |
| | | .-.G | | |
| 8212907 | 1686 | 86.-.C;131.A.C | 0.974581449 | 0.469863402 |
| 13097486 | 1687 | -1.GT.--;75.-.C | 0.974076361 | 0.347126982 |
| 3272148 | 1688 | 2.A.G;0.T.-;77.-.A | 0.973879721 | 0.592128628 |
| 8557995 | 1689 | 74.-.T;121.C.A | 0.973241728 | 0.209831785 |
| 8142576 | 1690 | 76.G.-;127.T.G | 0.972909535 | 0.375025867 |
| 14816291 | 1691 | -29.A.C;73.A.- | 0.971570292 | 0.231631239 |
| 10080185 | 1692 | 19.-.T89.-.C | 0.971142172 | 0.564636407 |
| 1904247 | 1693 | 0.TTA.-- | 0.970129816 | 0.748872279 |
| | | -;3.C.A;75.-.A | | |
| 6460821 | 1694 | 16.-.C;77.GA.-- | 0.969553741 | 0.637403652 |
| 12738126 | 1695 | 0.-.T;87.-.T | 0.968376883 | 0.57825455 |
| 8357730 | 1696 | 87.-.G;129.C.A | 0.968242916 | 0.269738584 |
| 12187919 | 1697 | 2.A.-;79.GA.-T | 0.968227596 | 0.963113501 |
| 14644862 | 1698 | - | 0.967299952 | 0.512413817 |
| | | 29.A.C;0.T.-;2.A.C;76 | | |
| | | .GG.-C | | |
| 13101334 | 1699 | -1.GT.--;76.GG.-T | 0.96664163 | 0.377178934 |
| 12437308 | 1700 | 1.TAC.---;80.A.- | 0.966358793 | 0.932816051 |
| 2672055 | 1701 | 0.T.-;2.A.C;86.C.A | 0.965996878 | 0.590376536 |
| 6304109 | 1702 | 16.-.A;76.GG.-C | 0.965683364 | 0.67187653 |
| 12214091 | 1703 | 2.A.-;73.A.T | 0.965610539 | 0.601810119 |
| 8511126 | 1704 | 76.6.-;78.AG.TC | 0.96509303 | 0.453545301 |
| 10473646 | 1705 | 16.C.-;76.GG.-T | 0.964836691 | 0.499237417 |
| 8561622 | 1706 | 74.-.T;82.A.- | 0.964731122 | 0.36234088 |
| 1981516 | 1707 | 0.T.C;75.C.- | 0.964349838 | 0.525063892 |
| 4300894 | 1708 | 4.T.-;77.G.T | 0.964207177 | 0.235903819 |
| 8084158 | 1709 | 74.-.G | 0.964116495 | 0.401532934 |
| 8096194 | 1710 | 75.-.A;87.-.T | 0.96360779 | 0.605413084 |
| 2281085 | 1711 | 0.T.-;87.C.T | 0.960523556 | 0.675358848 |
| 8063355 | 1712 | 74.T.-;86.-.C | 0.959756198 | 0.506555584 |
| 3038327 | 1713 | 1.TA.--;73.-.G | 0.9591209 | 0.853900434 |
| 9976817 | 1714 | 19.-.6;79.G.- | 0.958047025 | 0.737140085 |
| 13223005 | 1715 | 2.A.G;-3.TAGT.---- | 0.95795641 | 0.837056459 |
| 8542589 | 1716 | 75.-.6;98.-.T | 0.956947885 | 0.875376914 |
| 3345006 | 1717 | 0.T.-;2.A.G;73.A.T | 0.956723708 | 0.792775096 |
| 4217628 | 1718 | 4.T.-71.-.C | 0.956428726 | 0.494530665 |
| 10068711 | 1719 | 19.-.T;76.-.A | 0.955838642 | 0.689148232 |
| 10198139 | 1720 | 18.-.G;77.-.T | 0.95550711 | 0.662670415 |
| 2463484 | 1721 | 1.TA.--;3.C.A;87.-.T | 0.955371341 | 0.695396423 |
| 8490228 | 1722 | 76.-.6;128.T.G | 0.954993055 | 0.304520889 |
| 3322121 | 1723 | 0.T.-;2.A.G;80.AG.-T | 0.954883244 | 0.811714067 |
| 2458850 | 1724 | 1.TA.--;3.C.A;79.G.- | 0.954552438 | 0.857655704 |
| 6626017 | 1725 | 18.C.-;78.A.- | 0.954491633 | 0.61106783 |
| 8519520 | 1726 | 76.GG.-T;132.G.T | 0.954300925 | 0.281109543 |
| 1974653 | 1727 | 0.T.C;75.-.A | 0.954106906 | 0.489641158 |
| 2683428 | 1728 | 120.C.A;2.A.C;0.T.- | 0.953944451 | 0.252838081 |
| 4272200 | 1729 | 4.T.-;89.A.G | 0.953838275 | 0.924709618 |
| 8193481 | 1730 | 85.TC.-G | 0.952706766 | 0.701420781 |
| 6557686 | 1731 | 18.C.A;75.-.6 | 0.952635001 | 0.330369879 |
| 1860902 | 1732 | 0.TT.--;81.GA.-T | 0.952197311 | 0.514937583 |
| 2717874 | 1733 | 2.A.C;0.T.-;80.AG.-T | 0.951134819 | 0.611248832 |
| 2882024 | 1734 | 1.-.C;74.-.G | 0.950794893 | 0.618759103 |
| 3273132 | 1735 | 0.T.-;2.A.G;77.-.C | 0.95078631 | 0.397420244 |
| 441958 | 1736 | -27.C.A;76.GG.-A | 0.949448345 | 0.20486145 |
| 14811390 | 1737 | -29.A.C;78.A.- | 0.94924455 | 0.249151979 |
| 14802094 | 1738 | -29.A.C;86.-.C | 0.948918554 | 0.461499664 |
| 10523926 | 1739 | 15.-.T;76.-.A | 0.947880548 | 0.738861592 |
| 12742835 | 1740 | 0.-.T;81.GA.-T | 0.947825709 | 0.382500139 |
| 8093342 | 1741 | 75.-.A;133.A.C | 0.9477337 | 0.326505247 |
| 8490265 | 1742 | 76.-.G;129.C.A | 0.947716798 | 0.322105698 |
| 2412848 | 1743 | 1.-.A;76.-.T | 0.946977536 | 0.632308747 |
| 8183422 | 1744 | 85.TC.-A | 0.946704814 | 0.637809088 |
| 2463159 | 1745 | 1.TA.--;3.C.A;88.-.T | 0.945816148 | 0.551604962 |
| 8490433 | 1746 | 76.-.G,133.A.C | 0.94580569 | 0.317798446 |
| 2681222 | 1747 | 0.T.-;2.A.C;115.T.G | 0.945774394 | 0.287825585 |
| 8480741 | 1748 | 78.A.-;132.G.C | 0.945726636 | 0.201668102 |
| 2663534 | 1749 | 0.T.-;2.A.C;77.G.C | 0.945544637 | 0.860590156 |
| 8118132 | 1750 | 76.GG.-C;129.C.A | 0.94554045 | 0.373219502 |
| 6447398 | 1751 | 16.-.C;55.-.G | 0.945124875 | 0.768017164 |
| 2285156 | 1752 | 0.T.-;82.AA.-- | 0.94485704 | 0.502663519 |
| 8117520 | 1753 | 76.GG.-C;120.C.A | 0.944641128 | 0.413143505 |
| 8603147 | 1754 | 73.A.- | 0.944568512 | 0.225126189 |
| 8537609 | 1755 | 75.-.G;124.T.G | 0.944260148 | 0.365887334 |
| 2245955 | 1756 | 0.T.-;71.-.C | 0.944003192 | 0.683639716 |
| 8161116 | 1757 | 79.G.- | 0.942231169 | 0.264000452 |
| 8536998 | 1758 | 75.-.G;119.C.A | 0.941935837 | 0.370421962 |
| 8537871 | 1759 | 75.-.G;127.T.C | 0.941385669 | 0.333998494 |
| 8543767 | 1760 | 75.-.G;89.A.- | 0.94098922 | 0.627842945 |
| 6603080 | 1761 | 18.C.-;55.-.G | 0.940735855 | 0.707170754 |
| 13850293 | 1762 | -14.A.C;87.-.G | 0.939872328 | 0.218040413 |
| 1852615 | 1763 | 0.TT.--;76.-.A | 0.938499355 | 0.749884292 |
| 8208020 | 1764 | 88.G.-;132.G.C | 0.937909946 | 0.241574819 |
| 14918769 | 1765 | -29.A.C;2.A.-;76.GG.- | 0.937331761 | 0.352937114 |
| | | A | | |
| 8223161 | 1766 | 90.-.G | 0.936749506 | 0.664179652 |
| 2684123 | 1767 | 0.T.-;2.A.C;126.C.A | 0.935869575 | 0.26198456 |
| 2883487 | 1768 | 1.-.C;76.GG.-C | 0.934458485 | 0.884247882 |
| 8089075 | 1769 | 75.-C.AA | 0.934377668 | 0.299006427 |
| 13746840 | 1770 | -13.G.T;76.G.- | 0.934356994 | 0.266092099 |
| 10179608 | 1771 | 18.-.G;73.-.A | 0.933175531 | 0.586679061 |
| 8357113 | 1772 | 87.-.G;119.C.A | 0.933166453 | 0.238401775 |
| 2570963 | 1773 | 0.T.-;2.A.C;18.C.- | 0.93209533 | 0.403512556 |
| 6621548 | 1774 | 18.C.-;88.-.T | 0.931719159 | 0.702372684 |
| 8543544 | 1775 | 75.-.G;89.-.C | 0.93026646 | 0.330984722 |
| 8158269 | 1776 | 79.G.A | 0.928207937 | 0.859645581 |
| 3341556 | 1777 | 2.A.G;0.T.-;73.AT.-G | 0.928088432 | 0.857493258 |
| 2683151 | 1778 | 119.C.A;2.A.C;0.T.- | 0.927519705 | 0.28783831 |
| 8543919 | 1779 | 75.-.G;88.-.T | 0.925629705 | 0.543254506 |
| 2570189 | 1780 | 0.T.-;2.A.C;18.-.A | 0.925537001 | 0.64491759 |
| 4015474 | 1781 | 3.-.C;86.-.C | 0.925505786 | 0.838123078 |
| 2731496 | 1782 | 0.T.-;2.A.C;75.-.G;132 | 0.92511208 | 0.518018242 |
| | | .G.C | | |
| 8480834 | 1783 | 78.A.-;131.A.C | 0.925032194 | 0.257034431 |
| 3011827 | 1784 | 1.TA.-- | 0.923354091 | 0.387659338 |
| 8592843 | 1785 | 70.-.T;86.-.C | 0.923182623 | 0.500818269 |
| 8057655 | 1786 | 73.-.A | 0.923159152 | 0.547314306 |
| 8480787 | 1787 | 78.A.-;133.A.C | 0.922523853 | 0.246503981 |
| 2249456 | 1788 | 0.T.-;72.-.G | 0.922153962 | 0.819512544 |
| 8752628 | 1789 | 55.-.T;76.GG.-A | 0.92194028 | 0.502766206 |
| 2274200 | 1790 | 0.T.-;99.-.T | 0.92135973 | 0.847745604 |
| 8142972 | 1791 | 76.G.-;131.A.C;133.A. | 0.921146739 | 0.257676388 |
| | | C | | |
| 1252489 | 1792 | -15.T.G;76.GG.-T | 0.920958972 | 0.235680049 |
| 14822468 | 1793 | -29.A.C;55.-.T | 0.920816801 | 0.523726671 |
| 8357890 | 1794 | 87.-.G;131.A.C | 0.920798886 | 0.274644926 |
| 8485265 | 1795 | 76.-.G;88.G.- | 0.919513147 | 0.452533222 |
| 14796763 | 1796 | -29.A.C;74.-.C | 0.919493708 | 0.375134959 |
| 14796493 | 1797 | -29.A.C;74.T.- | 0.919211892 | 0.248759572 |
| 8558538 | 1798 | 74.-.T;133.A.C | 0.918860846 | 0.281318049 |
| 7247803 | 1799 | 27.-.C;86.CC.-G | 0.917956151 | 0.914761883 |
| 10073442 | 1800 | 19.-.T;88.GA.-C | 0.917769495 | 0.551828645 |
| 12133660 | 1801 | 2.A.-;85.TC.-G | 0.917554718 | 0.915961511 |
| 2572420 | 1802 | 0.T.-;2.A.C;19.-.A | 0.917245463 | 0.557634742 |
| 8555076 | 1803 | 74.-.T;88.G.- | 0.915485429 | 0.37741171 |
| 10607377 | 1804 | 16.C.T;75.-.G | 0.915305946 | 0.788886753 |
| 3281290 | 1805 | 2.A.G;0.T.-;88.G.- | 0.915191522 | 0.698541574 |
| 12713711 | 1806 | 0.-.T;72.-.A | 0.915132536 | 0.659473807 |
| 15408234 | 1807 | -30.C.G;0.T.-;2.A.C | 0.914828105 | 0.291008919 |
| 12722990 | 1808 | 0.-.T;79.G.- | 0.91469203 | 0.498534564 |
| 8105716 | 1809 | 76.GG.-A;132.G.T | 0.913542774 | 0.274934966 |
| 2271180 | 1810 | 0.T.- | 0.913216156 | 0.38072164 |
| 10289412 | 1811 | 17.-.T;90.-.G | 0.912848775 | 0.695466523 |
| 14807090 | 1812 | -29.A.C;87.-.T | 0.912395361 | 0.448815242 |
| 6108421 | 1813 | 14.-.A;72.-.C | 0.910081852 | 0.862648242 |
| 8141461 | 1814 | 76.G.-;119.C.A | 0.909297819 | 0.26332282 |
| 14350324 | 1815 | -25.A.C;76.-.C | 0.908340852 | 0.329528677 |
| 8538185 | 1816 | 130.-- | 0.906159692 | 0.420876967 |
| | | T.TAG;133.A.G;75.-. | | |
| | | G | | |
| 8538491 | 1817 | 75.-.G;123.A.C | 0.905622339 | 0.359184365 |
| 14292135 | 1818 | -25.A.C;0.T.-;2.A.C | 0.905462839 | 0.25526538 |
| 2399779 | 1819 | 1.-.A;75.-.C | 0.903712317 | 0.626250944 |
| 8142947 | 1820 | 76.G.-;131.AG.CC | 0.90278584 | 0.311578165 |
| 8603195 | 1821 | 73.A.-;131.A.C | 0.90153794 | 0.229442208 |
| 3329015 | 1822 | 2.A.G;0.T.-;78.-.T | 0.901071633 | 0.635158992 |
| 2457498 | 1823 | 1.TA.--;3.C.A;76.-.A | 0.90086193 | 0.877512785 |
| 14799938 | 1824 | -29.A.C;76.G.-;78.A.C | 0.900781085 | 0.250085624 |
| 10194359 | 1825 | 18.-.G;82.AA.-- | 0.900734628 | 0.723199799 |
| 2461767 | 1826 | 1.TA.--;3.C.A;99.-.G | 0.897938893 | 0.891247375 |
| 8128631 | 1827 | 75.-.C;131.AG.CC | 0.897742 | 0.298470213 |
| 6130904 | 1828 | 14.-.A;75.CG.-T | 0.897627082 | 0.808841286 |
| 2885480 | 1829 | 1.-.C;77.GA.-- | 0.896880771 | 0.563534094 |

TABLE 19

| index | SEQ ID NO | muts_lindexed | MI | 95% CI |
|---|---|---|---|---|
| 8565409 | 1830 | 131.A.C;75.CG.-T | 0.896200168 | 0.289353432 |
| 8526599 | 1831 | 76.-.T;133.A.C | 0.894753435 | 0.367051671 |
| 8542268 | 1832 | 75.-.G;99.-.G | 0.894634843 | 0.466299591 |
| 3296935 | 1833 | 0.T.-;2.A.G;98.-.T | 0.894142418 | 0.818628527 |
| 8535676 | 1834 | 115.T.G;75.-.G | 0.892450762 | 0.386408997 |
| 8530925 | 1835 | 75.-.G;82.-.A | 0.890548634 | 0.434402987 |
| 8142901 | 1836 | 76.G.-;134.G.T | 0.890248996 | 0.290204128 |
| 8142383 | 1837 | 76.G.-;125.T.G | 0.890028915 | 0.343416459 |
| 2054253 | 1838 | 0.TT.--;2.A.G;87.-.T | 0.889830012 | 0.871702087 |
| 8001281 | 1839 | 71.T.C | 0.887843685 | 0.608229078 |
| 6366788 | 1840 | 17.-.A;86.C.- | 0.887689243 | 0.797295445 |
| 12123821 | 1841 | 2.A.-;76.G.-;131.A.C | 0.886864617 | 0.302511684 |
| 15159066 | 1842 | -29.A.G;74.T.- | 0.88641859 | 0.227937789 |
| 10072842 | 1843 | 19.-.T;87.-.A | 0.886327606 | 0.611907237 |
| 1979426 | 1844 | 0.T.C;80.A.- | 0.885687199 | 0.575980831 |
| 10193667 | 1845 | 18.-.G;82.A.- | 0.885623931 | 0.827650358 |
| 1252039 | 1846 | -15.T.G;76.-.G | 0.885300041 | 0.316383221 |
| 4247573 | 1847 | 4.T.-;87.C.A | 0.885192731 | 0.526496586 |
| 6110295 | 1848 | 14.-.A;74.-.G | 0.883738665 | 0.833212815 |
| 6369429 | 1849 | 17.-.A;76.-.T | 0.883709542 | 0.672045707 |
| 6476407 | 1850 | 16.-.C;78.-.T | 0.883206478 | 0.612248822 |
| 2309043 | 1851 | 0.T.-;65.GC.-T | 0.88279209 | 0.648679211 |
| 10084280 | 1852 | 19.-.T;82.AA.-G | 0.882507854 | 0.749546575 |
| 2884850 | 1853 | 1.-.C;76.G.-;78.A.C | 0.881622675 | 0.491993778 |
| 2347258 | 1854 | 0.T.-;19.-.G | 0.879771208 | 0.615653289- |
| 12737110 | 1855 | 0.-.T;88.-.T | 0.879524619 | 0.357187729 |
| 10557558 | 1856 | 15.-.T;78.A.C | 0.878879263 | 0.710410533 |
| 1851901 | 1857 | 0.TT.--;74.-.G | 0.878121046 | 0.824086218 |
| 6621723 | 1858 | 18.C.-;86.C.- | 0.877071062 | 0.845236443 |
| 10567449 | 1859 | 15.-.T;73.A.G | 0.876199614 | 0.489297254 |
| 1863878 | 1860 | 0.TT.--;75.C.- | 0.876141036 | 0.766200413 |
| 7832261 | 1861 | 55.-.G;132.G.C | 0.875938665 | 0.806722857 |
| 15161180 | 1862 | -29.A.G;77.-.A | 0.875136509 | 0.216285884 |
| 8545164 | 1863 | 75.-.G;82.AA.-G | 0.875109059 | 0.568849243 |
| 7830386 | 1864 | 55.-.G;86.-.C | 0.874746244 | 0.74436841 |
| 6077749 | 1865 | 15.TC.-A;76.G.- | 0.874549453 | 0.859375029 |
| 8148008 | 1866 | 76.G.-;86.C.- | 0.87452541 | 0.186643953 |
| 2278635 | 1867 | 0.T.-;88.-.G | 0.873679439 | 0.724828094 |
| 1041817 | 1868 | -17.C.A;75.-.C | 0.873464925 | 0.245618671 |
| 2465231 | 1869 | 1.TA.--;3.C.A;82.AA.-T | 0.87288341 | 0.829692031 |
| 2266703 | 1870 | 0.T.-;90.-.G | 0.87219304 | 0.862449293 |
| 6625678 | 1871 | 18.C.-;78.-.C | 0.871854232 | 0.579835472 |
| 8136927 | 1872 | 76.G.-;86.-.C | 0.871633528 | 0.49310448 |
| 8093375 | 1873 | 75.-.A;131.A.C | 0.870605371 | 0.334695171 |
| 2454809 | 1874 | 1.TA.--;3.C.A;72.-.A | 0.870104785 | 0.7360795 |
| 1980576 | 1875 | 0.T.C;76.GG.-T | 0.870084283 | 0.466063377 |
| 2271158 | 1876 | 0.T.-;132.G.C | 0.869968206 | 0.382593755 |
| 442251 | 1877 | -27.C.A;75.-.C | 0.869789461 | 0.272812946 |
| 2350399 | 1878 | 0.T.-;18.-.G | 0.869175589 | 0.556109447 |
| 8498008 | 1879 | 78.A.G | 0.868791572 | 0.35574229 |
| 8080600 | 1880 | 74.-.G;86.-.C | 0.868096002 | 0.559804248 |
| 3328595 | 1881 | 2.A.G;0.T.-;78.AG.-T | 0.86801762 | 0.823575147 |
| 8467079 | 1882 | 78.AG.-C | 0.867519598 | 0.422260229 |
| 6459918 | 1883 | 16.-.C;77.-.A | 0.866086899 | 0.523207502 |
| 2265855 | 1884 | 0.T.-;88.GA.-C | 0.865179979 | 0.720694826 |
| 15161451 | 1885 | -29.A.G;79.G.- | 0.864880911 | 0.291402918 |
| 8565376 | 1886 | 75.CG.-T;133.A.C | 0.8647622 | 0.308122333 |
| 2684676 | 1887 | 0.T.-;2.A.C;131.A.G | 0.864125602 | 0.347136817 |
| 6461858 | 1888 | 16.-.C;86.-.A | 0.863837493 | 0.610729582 |
| 3011807 | 1889 | 1.TA.--;132.G.C | 0.863489882 | 0.395655463 |
| 1905700 | 1890 | 0.TTA.---;3.C.A;86.-.C | 0.86299387 | 0.79224794 |
| 8440297 | 1891 | 81.GAA.-TT | 0.862721887 | 0.410012308 |
| 8752800 | 1892 | 55.-.T;75.-.C | 0.862228765 | 0.546437409 |
| 12721020 | 1893 | 0.-.T75.-.C | 0.861994689 | 0.449429098 |
| 441780 | 1894 | -27.C.A;75.-.A | 0.861287307 | 0.299642761 |
| 10070497 | 1895 | 19.-.T;76.G.-;78.A.C | 0.861054294 | 0.561313263 |
| 8112403 | 1896 | 76.-.A;132.G.T | 0.860916867 | 0.583979668 |
| 1002534 | 1897 | -17.C.A;2.A.C;0.T.- | 0.860899766 | 0.227341425 |
| 3324612 | 1898 | 0.T.-;2.A.G;78.A.C | 0.86070632 | 0.73672108 |
| 3030912 | 1899 | 1.TA.--;78.A.-80.A.- | 0.860647782 | 0.838049368 |
| 10182195 | 1900 | 1 8.-.G;76.GG.-C | 0.860369871 | 0.461905865 |
| 8519380 | 1901 | 76.GG.-T;129.C.A | 0.860233343 | 0.206775628 |
| 8493521 | 1902 | 76.-.G;98.-.T | 0.859090878 | 0.735056688 |
| 8128428 | 1903 | 75.-.C;128.T.G | 0.857937673 | 0.24073509 |
| 1248006 | 1904 | -15.T.G;88.G.- | 0.856727 | 0.216712076 |
| 5585921 | 1905 | 10.T.C;76.G.- | 0.855093855 | 0.370550678 |
| 6127219 | 1906 | 14.-.A;78.A.- | 0.854883422 | 0.492926654 |
| 3007558 | 1907 | 1.TA.--;90.-.G | 0.854495024 | 0.711184832 |
| 10555821 | 1908 | 15.-.T;80.AG.-T | 0.854328412 | 0.84308171 |
| 12747339 | 1909 | 0.-.T;78.A.T | 0.853746444 | 0.745239398 |
| 14344892 | 1910 | -25.A.C;75.-.C | 0.853497099 | 0.295843322 |
| 10310038 | 1911 | 17.-.T;77.-.T | 0.853123635 | 0.646582684 |
| 4303315 | 1912 | 4.T.-;76.G.T | 0.851550244 | 0.664150686 |
| 14786751 | 1913 | -29.A.C;55.-.G | 0.851205863 | 0.737068985 |
| 15059318 | 1914 | -29.A.G;0.T.-;2.A.C;76.-.G | 0.851092115 | 0.284707875 |
| 15240190 | 1915 | -29.A.G;2.A.- | 0.850701999 | 0.499567732 |
| 6468525 | 1916 | 16.-.C;91.A.-;93.A.G | 0.848737138 | 0.651993977 |
| 2826831 | 1917 | 0.T.-;2.A.C;15.-.T;75.-.G | 0.848656876 | 0.523377407 |
| 8212871 | 1918 | 86.-.C;133.A.C | 0.848086579 | 0.669274383 |
| 3318144 | 1919 | 2.A.G;0.T.-;82.AA.-T | 0.847571377 | 0.741743097 |
| 1246180 | 1920 | -15.T.G;75.-.A | 0.847453607 | 0.337281833 |
| 1982591 | 1921 | 0.T.C;66.CT.-G | 0.84737962 | 0.441751749 |
| 15166880 | 1922 | -29.A.G;81.GA.-T | 0.847298283 | 0.253268693 |
| 1904171 | 1923 | 0.TTA.---;3.C.A;74.-.G | 0.845851242 | 0.783342801 |
| 14635061 | 1924 | -29.A.C;0.T.- | 0.845517511 | 0.38153428 |
| 8565091 | 1925 | 75.CG.-T;126.C.A | 0.845432049 | 0.207160773 |
| 2725821 | 1926 | 0.T.-;2.A.C;77.GA.--;80.A.T | 0.845151363 | 0.836702777 |
| 4259960 | 1927 | 4.T.-;130.T.G | 0.844420024 | 0.799710867 |
| 3135495 | 1928 | 1.T.G;3.C.-;75.-.G | 0.844345159 | 0.791310505 |
| 14345120 | 1929 | -25.A.C;76.G.- | 0.844207275 | 0.259459942 |
| 10071193 | 1930 | 19.-.T;81.G.- | 0.84366427 | 0.779495237 |
| 6476304 | 1931 | 16.-.C;78.AG.-T | 0.843608449 | 0.660829712 |
| 15175052 | 1932 | -29.A.G;55.-.T | 0.843589728 | 0.628713279 |
| 8519203 | 1933 | 76.GG.-T;126.C.A | 0.843115863 | 0.232539946 |
| 8173991 | 1934 | 77.GA.-- | 0.842982504 | 0.382878127 |
| 12746208 | 1935 | 0.-.T;76.-.G | 0.842187941 | 0.434677576 |
| 8133056 | 1936 | 75.-.C;87.-.T | 0.842005477 | 0.419078021 |
| 8526626 | 1937 | 76.-.T;131.A.0 | 0.841499516 | 0.222806303 |
| 1252968 | 1938 | -15.T.G;75.C.- | 0.840541627 | 0.361088873 |
| 14646713 | 1939 | -29.A.C;0.T.-;2.A.C;80.A.- | 0.840363457 | 0.512884706 |
| 6304778 | 1940 | 16.-.A;77.-.A | 0.839744987 | 0.461935208 |
| 8479746 | 1941 | 78.A.-;120.C.A | 0.838428917 | 0.292810002 |
| 12763666 | 1942 | 0.-.T;55.-.T | 0.838009445 | 0.783484132 |
| 2684656 | 1943 | 0.T.-;2.A.C;131.A.C;133.A.C | 0.837560227 | 0.206667086 |
| 14800177 | 1944 | -29.A.C;79.G.- | 0.837044741 | 0.233067105 |
| 8128118 | 1945 | 75.-.C;124.T.G | 0.836600946 | 0.256117965 |
| 13797685 | 1946 | -14.A.C;0.T.-;2.A.C | 0.836119439 | 0.249533999 |
| 4259801 | 1947 | 4.T.-;128.T.G | 0.836000745 | 0.762544053 |
| 6612829 | 1948 | 18.C.-;76.G.- | 0.833297918 | 0.707704073 |
| 448172 | 1949 | -27.C.A;73.A.- | 0.833152564 | 0.215681899 |
| 1246589 | 1950 | -15.T.G;76.GG.-C | 0.832838095 | 0.560142043 |
| 14796144 | 1951 | -29.A.C;73.-.A | 0.832196458 | 0.441116469 |
| 6611642 | 1952 | 18.C.-;76.GG.-A | 0.831495777 | 0.704158939 |
| 3040392 | 1953 | I .TA.--;73.A.T | 0.83125454 | 0.517209585 |
| 1938331 | 1954 | 0.TT.--;2.A.C;79.G.- | 0.83094649 | 0.782892584 |
| 10528065 | 1955 | 15.-.T;79.GA.-C | 0.830823439 | 0.713061332 |
| 3261986 | 1956 | 0.T.-;2.A.G;74.T.G | 0.82985054 | 0.735935966 |
| 8131593 | 1957 | 75.-.C;99.-.G | 0.829803923 | 0.552794831 |
| 14255597 | 1958 | -24.G.T;2.A.- | 0.829521014 | 0.569520648 |
| 14879001 | 1959 | -29.A.C;15.-.T;75.-.G | 0.829471291 | 0.804622726 |
| 14918841 | 1960 | -29.A.C;2.A.-;76.GG.-C | 0.829132035 | 0.731668707 |
| 2290589 | 1961 | 0.T.-;79.GA.-T | 0.828939315 | 0.726137312 |
| 2951795 | 1962 | 1.TA.--;16.-.0 | 0.828708264 | 0.305967101 |
| 9987799 | 1963 | 19.-.G;86.-.G | 0.827168874 | 0.730661257 |
| 15455726 | 1964 | -30.C.G;78.A.- | 0.827064513 | 0.282392503 |
| 14812695 | 1965 | -29.A.C;77.-.T | 0.826064557 | 0.574798815 |
| 8202480 | 1966 | 87.-.A;131.A.C | 0.825480268 | 0.570499479 |
| 8066107 | 1967 | 74.T.-;121.C.A | 0.824741856 | 0.204192194 |
| 14807234 | 1968 | -29.A.C;86.-.G | 0.823713381 | 0.173705555 |
| 10085211 | 1969 | 19.-.T;80.A.- | 0.823514146 | 0.633352874 |
| 8180233 | 1970 | 81.GA.-C | 0.823411608 | 0.427874666 |
| 1044371 | 1971 | -17.C.A;87.-.G | 0.821282659 | 0.292542788 |
| 10286908 | 1972 | 17.-.T;85.TC.-A | 0.821041632 | 0.501681072 |
| 10250881 | 1973 | 18.C.T;75.-.G | 0.820021901 | 0.593154858 |
| 2463586 | 1974 | 1.TA.--;3.0 A;86.-.G | 0.819988929 | 0.682384778 |
| 6554412 | 1975 | 18.C.A;76.G.- | 0.819014386 | 0.317795095 |
| 8485725 | 1976 | 76.-.G;98.-.A | 0.818075053 | 0.715764322 |
| 2271237 | 1977 | 0.T.-;131.A.C | 0.817142113 | 0.351930761 |
| 2564816 | 1978 | 0.T.-;2.A.C;17.-.A | 0.81646896 | 0.601217336 |
| 8357229 | 1979 | 87.-.G;120.C.A | 0.816184189 | 0.328957228 |
| 12747630 | 1980 | 0.-.T;76.G.-;78.A.T | 0.815905287 | 0.796115745 |
| 9972115 | 1981 | 19.-.G;73.-.A | 0.815790669 | 0.80208701 |
| 8212329 | 1982 | 86.-.C;121.C.A | 0.815247299 | 0.51423849 |
| 14654311 | 1983 | -29.A.C;1.TA.--;76.G.- | 0.815105862 | 0.379590045 |
| 1864798 | 1984 | 0.TT.--;73.AT.-G | 0.814459875 | 0.762293984 |
| 8117352 | 1985 | 76.GG.-C;119.C.A | 0.812998633 | 0.432977601 |
| 8479512 | 1986 | 78.A.-;119.C.A | 0.812335411 | 0.223689176 |
| 8133372 | 1987 | 75.-.C;82.A.- | 0.812332278 | 0.356824998 |
| 10468894 | 1988 | 16.C.-;87.-.G | 0.812035912 | 0.666965245 |
| 8489702 | 1989 | 76.-.G;121.C.A | 0.811977229 | 0.335430162 |
| 14919783 | 1990 | -29.A.C;2.A.- | 0.811812719 | 0.51274018 |
| 8198335 | 1991 | 86.C.A | 0.811151507 | 0.799145123 |
| 8105698 | 1992 | 76.GG.-A;133.A.C | 0.810854998 | 0.269366495 |
| 13845556 | 1993 | -14.A.C;76.GG.-C | 0.809202243 | 0.490618124 |
| 3011864 | 1994 | 1.TA.--;132.G.T | 0.80898504 | 0.35238499 |

TABLE 20

| | SEQ | | | |
|---|---|---|---|---|
| index | ID NO | muts_1indexed | MI | 95% CI |
| 13222066 | 1995 | 2.A.G;-3.TAGT.--- | 0.808611561 | 0.596822595 |
| | | -;76.GG.-A | | |
| 6471171 | 1996 | 16.-.C;82.A.- | 0.808494016 | 0.510086271 |
| 8526572 | 1997 | 132.G.C;76.-.T | 0.807564936 | 0.259100497 |
| 8352868 | 1998 | 86.C.-;131.A.C | 0.806885397 | 0.22636509 |
| 10198068 | 1999 | 18.-.G;76.G.-;78.A.T | 0.806835867 | 0.435582585 |
| 8137025 | 2000 | 76.G.-;89.-.A | 0.803563673 | 0.538455612 |
| 8629413 | 2001 | 66.CT.-G;88.G.- | 0.803450388 | 0.32031914 |
| 8105428 | 2002 | 76.GG.-A;126.C.A | 0.803147022 | 0.24041185 |
| 7947397 | 2003 | 66.CT.-A;87.-.G | 0.802024989 | 0.362070069 |
| 7835793 | 2004 | 55.-.G;76.GG.-T | 0.801885567 | 0.735401291 |
| 8140338 | 2005 | 76.G.-;116.T.G | 0.801593594 | 0.30577562 |
| 12722736 | 2006 | 0.-.T;77.-.C | 0.801221765 | 0.426859099 |
| 8757065 | 2007 | 55.-.T;86.C.- | 0.800987285 | 0.558821092 |
| 2398681 | 2008 | 1.-.A;75.-.A | 0.800763412 | 0.641433179 |
| 4011043 | 2009 | 3.-.C;74.-.C | 0.79937771 | 0.713346067 |
| 14920334 | 2010 | -29.A.C;2.A.-;86.C.- | 0.799161613 | 0.459738042 |
| 13845318 | 2011 | -14.A.C;76.GG.-A | 0.799099794 | 0.18794716 |
| 3427589 | 2012 | 0.T.-;2.A.G;19.-.G | 0.79900678 | 0.415960568 |
| 14806422 | 2013 | -29.A.C;89.A.- | 0.798118013 | 0.702122527 |
| 15165304 | 2014 | -29.A.G;87.-.T | 0.796830943 | 0.463308646 |
| 2125941 | 2015 | 0.TTA.-- | 0.796565821 | 0.79076485 |
| | | -;3.C.G;89.A.- | | |
| 15168973 | 2016 | -29.A.G;76.-.T | 0.796128601 | 0.380420766 |
| 8538239 | 2017 | 75.-.G;131.AG.CC | 0.795805651 | 0.429399788 |
| 8528721 | 2018 | 76.GGA.-TT | 0.795594742 | 0.447243511 |
| 7834109 | 2019 | 55.-.G;86.-.G | 0.794446595 | 0.595594758 |
| 8476335 | 2020 | 78.A.-;98.-.A | 0.793884665 | 0.527904732 |
| 8352802 | 2021 | 132.G.C;86.C.- | 0.793673627 | 0.214217899 |
| 10372832 | 2022 | 18.CA.-T;74.-.T | 0.793649001 | 0.724009478 |
| 8752727 | 2023 | 55.-.T;76.GG.-C | 0.792864878 | 0.681485029 |
| 6460172 | 2024 | 16.-.C;77.-.C | 0.792492284 | 0.473521838 |
| 1245743 | 2025 | -15.T.G;74.T.- | 0.792248453 | 0.347003397 |
| 6469515 | 2026 | 16.-.C88.-.T | 0.791786541 | 0.64480155 |
| 15241028 | 2027 | -29.A.G;2.A.-;78.A.- | 0.791581969 | 0.398369648 |
| 2711056 | 2028 | 0.T.-;2.A.C;82.A.G | 0.791084203 | 0.74717295 |
| 1974296 | 2029 | 0.T.C;74.T.- | 0.790042405 | 0.532969357 |
| 8637058 | 2030 | 66.CT.-G;86.-.G | 0.789170768 | 0.254255894 |
| 8526611 | 2031 | 76.-.T;132.G.T | 0.788188081 | 0.322643284 |
| 8144153 | 2032 | 76.G.-;119.C.T | 0.788021877 | 0.239807981 |
| 10566620 | 2033 | 15.-.T;73.A.C | 0.787853854 | 0.613069845 |
| 8557775 | 2034 | 74.-.T;119.C.A | 0.787787618 | 0.230477012 |
| 8462867 | 2035 | 79.GA.-T | 0.787274361 | 0.613395387 |
| 8549438 | 2036 | 75.C.- | 0.7872713 | 0.425057254 |
| 8558414 | 2037 | 74.-.T;129.C.A | 0.787235849 | 0.254942799 |
| 8105581 | 2038 | 76.GG.-A;129.C.A | 0.787085201 | 0.25915294 |
| 2281703 | 2039 | 0.T.-;86.C.T | 0.785739149 | 0.719182131 |
| 2400499 | 2040 | 1.-.A;76.G.-;78.A.C | 0.785147179 | 0.482179072 |
| 14920368 | 2041 | -29.A.C;2.A.-;87.-.G | 0.784869833 | 0.602095885 |
| 8543253 | 2042 | 75.-.G;91.A.-;93.A.G | 0.784852363 | 0.451551966 |
| 8488707 | 2043 | 76.-.G;116.T.G | 0.784670342 | 0.282512341 |
| 9979217 | 2044 | 19.-.G;86.-.C | 0.783235694 | 0.61177765 |
| 15162226 | 2045 | -29.A.G;86.-.A | 0.782740907 | 0.521792231 |
| 12146137 | 2046 | 2.A.-;116.T.G | 0.782680959 | 0.42917569 |
| 5454231 | 2047 | 8.G.C;76.G.- | 0.782380772 | 0.6463104 |
| 2288382 | 2048 | 0.T.-;77.GA.--;83.A.T | 0.781480078 | 0.648018195 |
| 8549424 | 2049 | 75.C.-;132.G.C | 0.781281893 | 0.386040689 |
| 6461529 | 2050 | 16.-.C;85.T.- | 0.781254783 | 0.720080877 |
| 1090544 | 2051 | 2.A.- | 0.781168584 | 0.530340013 |
| 2282648 | 2052 | 0.T.-;84.-.T | 0.779234454 | 0.667414229 |
| 12149194 | 2053 | 2.A.-;131.A.G | 0.778932674 | 0.43969611 |
| 8142223 | 2054 | 76.G.-;124.T.G | 0.778900279 | 0.273194276 |
| 8199575 | 2055 | 86.CC.-A | 0.77887351 | 0.610550764 |
| 13854291 | 2056 | -14.A.C;75.CG.-T | 0.778830352 | 0.362088557 |
| 8092813 | 2057 | 75.-.A;121.C.A | 0.778421275 | 0.281031479 |
| 8605540 | 2058 | 73.A.-;87.-.G | 0.778324817 | 0.302912081 |
| 68946 | 2059 | 0.T.-;2.A.C | 0.778217999 | 0.249763093 |
| 12199248 | 2060 | 2.A.-;76.GG.- | 0.778119212 | 0.423790052 |
| | | T;132.G.C | | |
| 8093073 | 2061 | 126.C.A75.-.A | 0.777970506 | 0.369671349 |
| 12149170 | 2062 | 2.A.-;131.A.C | 0.776491674 | 0.526766214 |
| 447600 | 2063 | -27.C.A;75.CG.-T | 0.776402867 | 0.266208398 |
| 8143156 | 2064 | 76.G.-;126.C.T | 0.776218375 | 0.345711065 |
| 1982252 | 2065 | 0.T.C;73.A.- | 0.776212517 | 0.440987509 |
| 4255522 | 2066 | 4.T.-;115.T.G | 0.776114871 | 0.763967165 |
| 8112417 | 2067 | 76.-.A;131.A.C | 0.776058906 | 0.677356656 |
| 8083653 | 2068 | 74.-.G121.C.A | 0.775457064 | 0.433721449 |
| 8539008 | 2069 | 75.-.G120.C.T | 0.775033077 | 0.360907809 |
| 13750813 | 2070 | -13.G.T;75.-.G | 0.773597076 | 0.496364906 |
| 8759144 | 2071 | 55.-.T;76.GG.-T | 0.77186309 | 0.578448287 |
| 2684637 | 2072 | 0.T.-;2.A.C;131.AG.C | 0.771368384 | 0.250615124 |
| | | C | | |
| 8032414 | 2073 | 72.-.C | 0.770653538 | 0.299141231 |
| 15165408 | 2074 | -29.A.G;86.-.G | 0.770467267 | 0.132165451 |
| 8352728 | 2075 | 86.C.-;129.C.A | 0.769563809 | 0.199735436 |
| 12191702 | 2076 | 2.A.-;78.A.-;131.A.C | 0.768623982 | 0.496502512 |
| 12751144 | 2077 | 0.-.T;74.-.T | 0.76856622 | 0.416724498 |
| 2894079 | 2078 | 1.-.C;87.-.G | 0.76797859 | 0.69721306 |
| 8480622 | 2079 | 78.A.-;129.C.A | 0.767578125 | 0.331587077 |
| 8758901 | 2080 | 55.-.T;76.-.G | 0.766343494 | 0.641541627 |
| 8202090 | 2081 | 87.-.A;121.C.A | 0.766102496 | 0.622079897 |
| 2885067 | 2082 | 1.-.C;79.G.- | 0.765626173 | 0.51214927 |
| 8202431 | 2083 | 87.-.A;132.G.C | 0.765077306 | 0.53718099 |
| 12191659 | 2084 | 2.A.-;78.A.-;132.G.C | 0.764704817 | 0.595721144 |
| 12149115 | 2085 | 2.A.-;133.A.C | 0.764324854 | 0.438594709 |
| 2271200 | 2086 | 0.T.-;133.A.C | 0.763753757 | 0.4294745 |
| 2252404 | 2087 | 0.T.-;74.T.G | 0.763452663 | 0.476144264 |
| 8142993 | 2088 | 131.A.G;76.G.- | 0.761824261 | 0.24967661 |
| 446438 | 2089 | -27.C.A;78.A.- | 0.761792637 | 0.249126858 |
| 8480581 | 2090 | 78.A.-;128.T.G | 0.76178249 | 0.28018538 |
| 3133382 | 2091 | 1.T.G;3.C.-;74.-.G | 0.760891826 | 0.629329233 |
| 2302762 | 2092 | 0.T.-73.A.G | 0.760848385 | 0.618073183 |
| 1041081 | 2093 | -17.C.A;74.T.- | 0.760237431 | 0.229813983 |
| 1074428 | 2094 | -17.C.A;2.A.- | 0.759954307 | 0.561101375 |
| 10571409 | 2095 | 15.-.T65.GC.-T | 0.759803199 | 0.638728683 |
| 8598575 | 2096 | 70.-.T;86.C.- | 0.757656592 | 0.3746533 |
| 8363306 | 2097 | 87.-.T;131.A.C | 0.757331721 | 0.451839871 |
| 8143881 | 2098 | 76.G.-;120.C.T | 0.757192938 | 0.313345954 |
| 15159530 | 2099 | -29.A.G;74.-.G | 0.757082564 | 0.394186622 |
| 4230077 | 2100 | 4.T.-;75.C.A | 0.755983607 | 0.733464455 |
| 8146649 | 2281 | 76.G.-;99.-.G | 0.755070921 | 0.379444158 |
| 2684498 | 2282 | 0.T.-,2.A.C,130.T.G | 0.754689937 | 0.294762457 |
| 8128273 | 2283 | 75.-.C126.C.A | 0.753949302 | 0.276623271 |
| 8066406 | 2284 | 74.T.-;126.C.A | 0.751660833 | 0.236816233 |
| 8363243 | 2285 | 87.-.T;132.G.C | 0.751028711 | 0.468864036 |
| 8142864 | 2286 | 76.G.-;132.GA.CC | 0.750861564 | 0.275934907 |
| 2512825 | 2287 | 1.T.C;76.G.- | 0.7504689 | 0.48593163 |
| 8091801 | 2288 | 75.-.A;115.T.G | 0.749700204 | 0.260297227 |
| 1114939 | 2289 | -16.C.A;76.G.- | 0.749305598 | 0.263900263 |
| 8142311 | 2290 | 76.G.-;125.T.C | 0.74877691 | 0.290550934 |
| 11774438 | 2291 | 2.-.C;76.GG.-A | 0.748308714 | 0.657502587 |
| 15064284 | 2292 | -29.A.G;1.TA.-- | 0.748045422 | 0.3832171 |
| 1187746 | 2293 | -15.T.G;0.T.- | 0.748017281 | 0.384223169 |
| 8092581 | 2294 | 75.-.A;119.C.A | 0.746934248 | 0.329723696 |
| 1246493 | 2295 | -15.T.G;76.-.A | 0.746842913 | 0.493140906 |
| 14646216 | 2296 | - | 0.74668829 | 0.368724428 |
| | | 29.A.C;0.T.-;2.A.C;87 | | |
| | | .-.G | | |
| 8142526 | 2297 | 76.G.-;127.T.C | 0.74638204 | 0.249355712 |
| 8191621 | 2298 | 85.TCC.-GA | 0.745990957 | 0.478821582 |
| 10308897 | 2299 | 17.-.T;78.A.G | 0.74547438 | 0.691042832 |
| 14661314 | 2300 | - | 0.745107888 | 0.569801975 |
| | | 29.A.C;0.T.-;2.A.G;75 | | |
| | | .-.C | | |
| 8549337 | 2301 | 75.C.-;129.C.A | 0.745005935 | 0.299426299 |
| 8753061 | 2302 | 55.-.T;79.G.- | 0.744926149 | 0.513566692 |
| 10097262 | 2303 | 19.-.T;55.-.T | 0.744819737 | 0.582631114 |
| 8161158 | 2304 | 79.G.-;131.A.C | 0.743647218 | 0.214645028 |
| 2661991 | 2305 | 0.T.-;2.A.C;76.G.-;131 | 0.743411308 | 0.431940993 |
| | | .A.C | | |
| 9987131 | 2306 | 19.-.G;86.C.- | 0.74325326 | 0.684101481 |
| 1046156 | 2307 | -17.C.A;76.GG.-T | 0.742891912 | 0.206153413 |
| 3311900 | 2308 | 0.T.-;2.A.G;83.-.C | 0.742731517 | 0.541403805 |
| 2412608 | 2309 | 1.-.A;76.GG.-T | 0.7419989 | 0.454493748 |
| 8092717 | 2310 | 75.-.A;120.C.A | 0.740460814 | 0.353030203 |
| 2684366 | 2311 | 0.T.-;2.A.C;128.T.G | 0.740365485 | 0.319772226 |
| 8536239 | 2312 | 75.-.G;116.T.G | 0.739558614 | 0.409490289 |
| 8483990 | 2313 | 78.A.-;98.-.T | 0.738582774 | 0.635321715 |
| 1290147 | 2314 | -15.T.G;2.A.-;76.G.- | 0.736953498 | 0.358146051 |
| 8629656 | 2315 | 66.CT.-G;89.-.A | 0.736647742 | 0.643898592 |
| 8039677 | 2316 | 72.-.G;86.-.C | 0.736394521 | 0.628402188 |
| 8528174 | 2317 | 76.-.T;87.-.G | 0.736315801 | 0.316059266 |
| 8142772 | 2318 | 76.G.-;130.T.C | 0.735973311 | 0.349764548 |
| 12148593 | 2319 | 2.A.-;126.C.A | 0.735792991 | 0.540631906 |
| 8089812 | 2320 | 75.-.A;88.G.- | 0.735648884 | 0.621749821 |
| 8436907 | 2321 | 81.GA.-T;131.A.C | 0.734237962 | 0.289458336 |
| 6303279 | 2322 | 16.-.A;74.-.G | 0.732956994 | 0.70590626 |
| 8136856 | 2323 | 76.G.-;88.G.- | 0.732170571 | 0.393401019 |
| 13099840 | 2324 | -1.GT.--;87.-.G | 0.73213014 | 0.204923163 |
| 12147390 | 2325 | 2.A.-;119.C.A | 0.731356849 | 0.364446154 |
| 8480707 | 2326 | 78.A.-;130.T.G | 0.730801992 | 0.306613853 |
| 8145151 | 2327 | 76.G.-;113.A.C | 0.729155512 | 0.24017937 |
| 2682115 | 2328 | 116.T.G;2.A.C;0.T.- | 0.726372083 | 0.269099758 |
| 2397740 | 2329 | 1.-.A;73.-.A | 0.725232042 | 0.569675223 |
| 8477975 | 2330 | 78.A.-;115.T.G | 0.725003641 | 0.25829691 |
| 10190335 | 2331 | 18.-.G;99.-.G | 0.724967082 | 0.471801343 |
| 15456232 | 2332 | -30.C.G;76.GG.-T | 0.724648029 | 0.153274083 |
| 1191613 | 2333 | - | 0.723562149 | 0.39593116 |
| | | 15.T.G;0.T.-;2.A.C;76. | | |
| | | G.- | | |
| 8352265 | 2334 | 86.C.-;121.C.A | 0.72284596 | 0.142245465 |
| 8212804 | 2335 | 86.-.C;130.T.G | 0.721964157 | 0.480722755 |
| 8549476 | 2336 | 132.G.T;75.C.- | 0.721079989 | 0.389979571 |
| 9994620 | 2337 | I9.-.G;77-.T | 0.720984013 | 0.612544282 |
| 14350752 | 2338 | -25.A.C;76.GG.-T | 0.720650806 | 0.13185545 |
| 13099030 | 2339 | -1.GT.-- | 0.72055901 | 0.376134358 |

TABLE 21

| | SEQ | | | |
|---|---|---|---|---|
| index | ID NO | muts_1indexed | MI | 95% CI |
| 12147928 | 2340 | 2.A.-;121.C.A | 0.720545241 | 0.487545739 |
| 1253117 | 2341 | -15.T.G;74.-.T | 0.720084866 | 0.252501472 |
| 8208073 | 2342 | 88.G.-;131.A.C | 0.719133155 | 0.210050353 |
| 2684254 | 2343 | 0.T.-;2.A.C;127.T.G | 0.719036934 | 0.352679314 |
| 8154688 | 2344 | 76.G.-;78.A.C;132.G. | 0.718994464 | 0.383020798 |
| | | C | | |
| 318717 | 2345 | -28.G.C;76.G.- | 0.71885563 | 0.191720408 |
| 8142885 | 2346 | 130.-- | 0.718716342 | 0.300945926 |
| | | T.TAG;133.A.G;76.G. | | |
| | | - | | |
| 14687527 | 2347 | -29.A.C;4.T.-;78.A.- | 0.71775509 | 0.526752246 |
| 15162677 | 2348 | -29.A.G;89.-.A | 0.717702888 | 0.668207942 |
| 15450951 | 2349 | -30.C.G;76.GG.-C | 0.717140275 | 0.47685517 |
| 8405267 | 2350 | 82.AA.-- | 0.715989547 | 0.291686385 |
| 8066712 | 2351 | 74.T.-;132.G.T | 0.715629569 | 0.310262393 |
| 8112393 | 2352 | 76.-.A;133.A.C | 0.71549299 | 0.479861009 |
| 8564706 | 2353 | 75.CG.-T,120.C.A | 0.714963297 | 0.236535754 |
| 8538090 | 2354 | 75.-.G;130.T.C | 0.714585785 | 0.385707956 |
| 14081174 | 2355 | -20.A.C;76.G.- | 0.714441554 | 0.176857594 |
| 8357562 | 2356 | 87.-.G;126.C.A | 0.713356322 | 0.284696561 |
| 6476171 | 2357 | 16.-.C;78.A.G | 0.713329524 | 0.676881239 |
| 12145038 | 2358 | 2.A.-;115.T.G | 0.712513 | 0.523524776 |
| 8636717 | 2359 | 66.CT.-G;88.-.T | 0.712296212 | 0.372467895 |
| 8208060 | 2360 | 88.G.-;132.G.T | 0.712226175 | 0.261444904 |
| 2746161 | 2361 | 0.T.-;2.A.C;66.CT.- | 0.711241204 | 0.361583276 |
| | | G;132.G.0 | | |
| 8064859 | 2362 | 74.T.-;115.T.G | 0.710992569 | 0.209965515 |
| 1981797 | 2363 | 0.T.C;75.CG.-T | 0.710765302 | 0.646448886 |
| 15719823 | 2364 | -32.G.T;0.T.-;2.A.C | 0.710088606 | 0.271097621 |
| 3024059 | 2365 | 1.TA.--;82.AA.-C | 0.709917185 | 0.373332434 |
| 14806152 | 2366 | -29.A.C;89.-.C | 0.708940534 | 0.181536327 |
| 14634677 | 2367 | -29.A.C;0.T.-;76.G.- | 0.708441715 | 0.420617475 |
| 672656 | 2368 | -23.C.A;75.-.G | 0.708188696 | 0.429780424 |
| 8628797 | 2369 | 66.CT.-G;77.GA.-- | 0.707896801 | 0.333142814 |
| 10529623 | 2370 | 15.-.T;85.TC.-A | 0.70783661 | 0.506178761 |
| 10196969 | 2371 | 18.-.G;78.A.- | 0.707389309 | 0.69751051 |
| 8057272 | 2372 | 73.-.A;121.C.A | 0.707360184 | 0.369603218 |
| 13845728 | 2373 | -14.A.C;75.-.C | 0.706574477 | 0.296568536 |
| 1045822 | 2374 | -17.C.A;76.-.G | 0.706174615 | 0.323551014 |
| 10460865 | 2375 | 16.C.-;76.GG.-C | 0.705744149 | 0.522507616 |
| 4222138 | 2376 | 4.T.-;72.-.G | 0.704993477 | 0.401332431 |
| 1152457 | 2377 | -15.T.C;0.T.-;2.A.C | 0.704466347 | 0.351046476 |
| 8069945 | 2378 | 74.T.-;87.-.T | 0.70432033 | 0.402131002 |
| 6303440 | 2379 | 16.-.A;75.-.A | 0.704295633 | 0.656523061 |
| 5593794 | 2380 | 10.T.C;75.CG.-T | 0.704113278 | 0.280887784 |
| 14654654 | 2381 | -29.A.C;1.TA.-- | 0.703489272 | 0.363240543 |
| 7829345 | 2382 | 55.-.G;76.GG.-C | 0.703371081 | 0.651218332 |
| 7490581 | 2383 | 36.C.A;76.GG.-C | 0.702828956 | 0.438837246 |
| 15452184 | 2384 | -30.C.G;86.-.C | 0.702460521 | 0.465360303 |
| 8089736 | 2385 | 75.-.A;87.-.A | 0.702242786 | 0.403569437 |
| 3161365 | 2386 | 0.T.-;2.A.G;14.-.A | 0.702180409 | 0.699897723 |
| 8215458 | 2387 | 88.GA.-C | 0.702027917 | 0.285995925 |
| 2455947 | 2388 | 1.TA.--;3.C.A;73.-.A | 0.70199884 | 0.692587003 |
| 827787 | 2389 | -21.C.A;76.G.- | 0.701801158 | 0.246155238 |
| 3574182 | 2390 | 2.-.A;55.-.G | 0.70077073 | 0.681126044 |
| 8504697 | 2391 | 78.-.T | 0.700694002 | 0.457301016 |
| 8147538 | 2392 | 76.G.-;91.A.-;93.A.G | 0.700512042 | 0.391148044 |
| 8436856 | 2393 | 81.GA.-T;132.G.C | 0.700344125 | 0.19857296 |
| 8110287 | 2394 | 76.-.A;86.-.C | 0.700322656 | 0.448259352 |
| 8598693 | 2395 | 70.-.T;87.-.T | 0.699981587 | 0.315205095 |
| 4260194 | 2396 | 4.T.-;129.C.T | 0.699010018 | 0.509569637 |
| 8059622 | 2397 | 73.-.A;87.-.G | 0.698999314 | 0.388603932 |
| 8586230 | 2398 | 73.AT.-G | 0.698732941 | 0.264987891 |
| 8126524 | 2399 | 75.-.C;115.T.G | 0.698610242 | 0.336087672 |
| 10084621 | 2400 | 19.-.T;82.AA.-T | 0.698526311 | 0.642093957 |
| 10607021 | 2401 | 16.C.T;78.A.- | 0.698487586 | 0.567347419 |
| 8212230 | 2402 | 86.-.C;120.C.A | 0.698013662 | 0.50513075 |
| 2664493 | 2403 | 0.T.-;2.A.C;79.G.A | 0.698011945 | 0.639630835 |
| 2203429 | 2404 | 0.T.-;18.C.- | 0.697561122 | 0.407203853 |
| 8605503 | 2405 | 73.A.,-;86.C.- | 0.697298567 | 0.200410632 |
| 13852662 | 2406 | -14.A.C;78.A.- | 0.697272825 | 0.309315646 |
| 8546163 | 2407 | 75.C.-;86.-.C | 0.697016055 | 0.445359301 |
| 446575 | 2408 | -27.C.A;76.-.G | 0.695980214 | 0.351410771 |
| 8065997 | 2409 | 74.T.-;120.C.A | 0.695979977 | 0.233779111 |
| 11888602 | 2410 | 2.A.C;75.-.G | 0.69559201 | 0.514633776 |
| 8536608 | 2411 | 75.-.G;118.T.C | 0.693904103 | 0.323497498 |
| 14797194 | 2412 | -29.A.C;74.-.G | 0.693690739 | 0.384361164 |
| 15166776 | 2413 | -29.A.G;82.AA.-T | 0.693594042 | 0.237378116 |
| 14800643 | 2414 | -29.A.C;77.GA.-- | 0.693435682 | 0.378778787 |
| 8030604 | 2415 | 72.-.C;86.-.C | 0.692063669 | 0.344818271 |
| 2464748 | 2416 | 1.TA.--;3.C.A;82.AA.- | 0.691743005 | 0.573710339 |
| | | C | | |
| 8493269 | 2417 | 76.-.G;99.-.G | 0.691472756 | 0.355929538 |
| 8549456 | 2418 | 75.C.-;133.A.C | 0.69071559 | 0.458090894 |
| 2307776 | 2419 | 0.T.-;66.CT.-- | 0.690358826 | 0.673270196 |
| 6306305 | 2420 | 16.-.A;86.-.C | 0.690314014 | 0.602110134 |
| 8126956 | 2421 | 75.-.C;116.T.G | 0.690175397 | 0.277812588 |
| 14809754 | 2422 | -29.A.C;81.GA.-T | 0.688454834 | 0.29609246 |
| 8212714 | 2423 | 86.-.C;128.T.G | 0.687830213 | 0.369390789 |
| 1251890 | 2424 | -15.T.G;78.A.- | 0.68686342 | 0.318568855 |
| 8518607 | 2425 | 76.GG.-T;119.C.A | 0.68650775 | 0.191235812 |
| 8057702 | 2426 | 73.-.A;131.A.C | 0.686176201 | 0.431944832 |
| 3024866 | 2427 | 1.TA.--;82.AA.-G | 0.686104906 | 0.454012439 |
| 8367599 | 2428 | 86.-.G;133.A.C | 0.68587266 | 0.156982412 |
| 8431922 | 2429 | 82.AA.-T | 0.685861849 | 0.217270657 |
| 8144351 | 2430 | 76.G.-;117.G.T | 0.685412598 | 0.238848867 |
| 8538257 | 2431 | 75.-.G;131.A.C;133.A. | 0.685222941 | 0.418849067 |
| | | C | | |
| 8543064 | 2432 | 75.-.G;91.A.- | 0.684684899 | 0.640360013 |
| 15455856 | 2433 | -30.C.G;76.-.G | 0.684667278 | 0.299094636 |
| 12149015 | 2434 | 2.A.-;130.T.G | 0.684628303 | 0.459482563 |
| 2685087 | 2435 | 0.T.-;2.A.C;122.A.C | 0.68431304 | 0.234414414 |
| 8084140 | 2436 | 74.-.G;132.G.C | 0.683463073 | 0.395894389 |
| 8142757 | 2437 | 76.G.-;130.T.C;132.G. | 0.683368549 | 0.271903521 |
| | | C | | |
| 8538197 | 2438 | 75.-.G;134.G.T | 0.683303537 | 0.367656483 |
| 15058053 | 2439 | - | 0.683089038 | 0.335849266 |
| | | 29.A.G;0.T.-;2.A.C;76 | | |
| | | .GG.-C | | |
| 8066567 | 2440 | 74.T.-;129.C.A | 0.680987394 | 0.26636043 |
| 441402 | 2441 | -27.C.A;74.T.- | 0.680666111 | 0.300414617 |
| 1042785 | 2442 | -17.C.A;86.-.0 | 0.678600413 | 0.334671562 |
| 8490149 | 2443 | 76.-.G;127.T.G | 0.678408907 | 0.29278641 |
| 1905560 | 2444 | 0.TTA.-- | 0.678221748 | 0.634547551 |
| | | -;3.C.A;87.-.A | | |
| 8352170 | 2445 | 86.C.-;120.C.A | 0.678142556 | 0.182223647 |
| 1252598 | 2446 | -15.T.G;76.-.T | 0.677678067 | 0.234976145 |
| 2400384 | 2447 | 1.-.A;77.-.A | 0.677524672 | 0.355978788 |
| 8087722 | 2448 | 74.-.G;86.C.- | 0.676149479 | 0.432474934 |
| 8101522 | 2449 | 75.-C.AG | 0.67614354 | 0.285448934 |
| 8087834 | 2450 | 74.-.G;87.-.T | 0.676028279 | 0.449497639 |
| 8431908 | 2451 | 82.AA.-T;132.G.C | 0.675935187 | 0.224923092 |
| 14645411 | 2452 | - | 0.675701823 | 0.635118105 |
| | | 29.A.C;0.T.-;2.A.C;86 | | |
| | | .-.C | | |
| 2835829 | 2453 | 0.T.-;2.A.C;6.G.T | 0.674847549 | 0.297866453 |
| 8438736 | 2454 | 81.GAA.-TC | 0.674319631 | 0.36029861 |
| 8065838 | 2455 | 74.T.-;119.C.A | 0.673352621 | 0.209456007 |
| 15171004 | 2456 | -29.A,G;73.A.- | 0.67309218 | 0.259465148 |
| 8084203 | 2457 | 74.-.G;131.A.C | 0.672638793 | 0.327011811 |
| 15161712 | 2458 | -29.A.G;77.GA.-- | 0.672345803 | 0.38770658 |
| 6613064 | 2459 | 18.C.-;77.-.A | 0.672260517 | 0.550699573 |
| 12315000 | 2460 | 2.A.-;15.-.T;75.-.G | 0.672180697 | 0.634716358 |
| 14246167 | 2461 | -24.G.T;75.-.G | 0.671730114 | 0.307720749 |
| 15051656 | 2462 | -29.A.G;0.T.- | 0.67119501 | 0.366366001 |
| 8469914 | 2463 | 78.-.C;121.C.A | 0.670982816 | 0.231982774 |
| 8352836 | 2464 | 86.C.-;133.A.C | 0.670437953 | 0.207264383 |
| 8554990 | 2465 | 74.-.T;87.-.A | 0.670240877 | 0.490358551 |
| 830076 | 2466 | -21.C.A;75.-.G | 0.670218516 | 0.422319746 |
| 8538376 | 2467 | 75.-.G;126.C.G | 0.670202704 | 0.370287506 |
| 15451096 | 2468 | -30.C.G;75.-.C | 0.670027612 | 0.235695956 |
| 1290476 | 2469 | -15.T.G;2.A.- | 0.668606404 | 0.65790079 |
| 14644913 | 2470 | - | 0.667729957 | 0.334589988 |
| | | 29.A.C;0.T.-;2.A.C;75 | | |
| | | .-.C | | |
| 8481064 | 2471 | 78.A.-;123.A.C | 0.666590429 | 0.232012003 |
| 12726534 | 2472 | 0.-.T;86.-.C | 0.665708352 | 0.531149931 |
| 14814019 | 2473 | -29.A.C;75.C.- | 0.665656435 | 0.396720553 |
| 15450607 | 2474 | -30.C.G;75.-.A | 0.665082103 | 0.225224942 |
| 8512477 | 2475 | 76.G.-;78.A.T;132.G. | 0.665001481 | 0.478100918 |
| | | C | | |
| 1247921 | 2476 | -15.T.G;87.-.A | 0.664815358 | 0.476053218 |
| 6461965 | 2477 | 16.-.C;86.CC.-A | 0.663795788 | 0.62018675 |
| 14815751 | 2478 | -29.A.C;73.A.G | 0.663422519 | 0.362091839 |
| 8557906 | 2479 | 74.-.T;120.C.A | 0.663111331 | 0.196201718 |
| 8174025 | 2480 | 77.GA --;132.G.T | 0.662605083 | 0.264797557 |
| 1979872 | 2481 | 0.T.C;78.-.C | 0.662557174 | 0.404196186 |
| 8148116 | 2482 | 76.G.-;87.-.T | 0.662403165 | 0.583645084 |
| 8055441 | 2483 | 73.-.A;86.-.C | 0.662135274 | 0.470696085 |
| 15162449 | 2484 | -29.A.G;88.G.- | 0.66196323 | 0.205534263 |
| 8522485 | 2485 | 76.GGA.-TC | 0.66191775 | 0.401082807 |
| 3081068 | 2486 | 1.TA.--;18.-.G | 0.661511132 | 0.556336464 |
| 8117952 | 2487 | 76.GG.-C;126.C.A | 0.661310322 | 0.38129357 |
| 6469397 | 2488 | 16.-.C;89.-.T | 0.661127615 | 0.591422391 |
| 8181855 | 2489 | 85.TCC.-AA | 0.661004434 | 0.567631116 |
| 1044315 | 2490 | -17.C.A;86.C.- | 0.660954164 | 0.167201347 |
| 14920528 | 2491 | -29.A.C;2.A.-;82.A.- | 0.659413017 | 0.536093731 |
| 8518772 | 2492 | 76.GG.-T;120.C.A | 0.65901063 | 0.283077251 |
| 15058093 | 2493 | - | 0.658082073 | 0.434010427 |
| | | 29.A.G;0.T.-;2.A.C;75 | | |
| | | .-.C | | |
| 8057683 | 2494 | 132.G.T;73.-.A | 0.656683021 | 0.433937068 |
| 2459622 | 2495 | 1.TA.--;3.C.A;86.-.A | 0.656221452 | 0.656035224 |
| 8069836 | 2496 | 74.T.-;86.C.- | 0.655888245 | 0.292848962 |
| 3320802 | 2497 | 2.A.G;0.T.-;80.A.- | 0.655685526 | 0.611479278 |
| 14919186 | 2498 | -29.A.C;2.A.-;77.GA.- | 0.655286056 | 0.360298823 |
| 8207846 | 2499 | 88.G.-;126.C.A | 0.655096377 | 0.243604744 |
| 447068 | 2500 | -27.C.A;76.-.T | 0.65455178 | 0.227422314 |
| 8603132 | 2501 | 73.A.-;132.G.C | 0.653928447 | 0.247296366 |
| 8755264 | 2502 | 55.-.T;132.G.C | 0.653511089 | 0.548281641 |
| 443309 | 2503 | -27.C.A;86.-.C | 0.653207249 | 0.447236787 |

TABLE 22

| | SEQ | | | |
|---|---|---|---|---|
| index | ID NO | muts_lindexed | MI | 95% CI |
| 8548846 | 2504 | 75.C.-;121.C.A | 0.652717251 | 0.454635257 |
| 8150297 | 2505 | 77.-.A;132.G.T | 0.652483401 | 0.274067745 |
| 8603165 | 2506 | 73.A.-;133.A.C | 0.651995199 | 0.297596 |
| 12312790 | 2507 | 16.C.-;2.A.- | 0.651829339 | 0.523664364 |
| 10248608 | 2508 | 18.C.T;76.G.- | 0.65143407 | 0.536447137 |
| 1046713 | 2509 | -17.C.A;75.CG.-T | 0.651373242 | 0.2628061 |
| 8638044 | 2510 | 66.CT.-G;82.AA.-T | 0.651267731 | 0.286853587 |
| 3315325 | 2511 | 0.T.-;2.A.G;82.AA.-C | 0.649742268 | 0.60527814 |
| 12314014 | 2512 | 2.A.-;15.-.T;76.G.- | 0.649432547 | 0.573783459 |
| 8494400 | 2513 | 76.-.G;86.C.- | 0.649382925 | 0.187112086 |
| 14920881 | 2514 | -29.A.C;2.A.-;80.A.- | 0.648202591 | 0.517031462 |
| 14243707 | 2515 | -24.G.T;76.G.- | 0.647505918 | 0.184867776 |
| 12148911 | 2516 | 2.A.-;129.C.A | 0.646912178 | 0.60106697 |
| 12149062 | 2517 | 2.A.-132.G.C | 0.646447274 | 0.501642261 |
| 8600526 | 2518 | 73.A.-;88.G.- | 0.645193272 | 0.440415837 |
| 8538871 | 2519 | 75.-.G;121.C.T | 0.645184704 | 0.40216231 |
| 8603181 | 2520 | 73.A.-;132.G.T | 0.645084394 | 0.288944622 |
| 15450764 | 2521 | -30.C.G;76.GG.-A | 0.644258092 | 0.211001918 |
| 12149230 | 2522 | 2.A.-;129.C.G | 0.643329654 | 0.340406439 |
| 8558338 | 2523 | 74.-.T;127.T.G | 0.643068363 | 0.272440562 |
| 8367575 | 2524 | 86.-.G;132.G.C | 0.641668887 | 0.1457948 |
| 14647726 | 2525 | -29.A.C;0.T.-;2.A.C;66.CT.-G | 0.641412285 | 0.377955569 |
| 8490463 | 2526 | 76.-.G;131.AG.CC | 0.640049069 | 0.222285584 |
| 12123507 | 2527 | 2.A.-;76.G.-;121.C.A | 0.639903685 | 0.451876032 |
| 8352850 | 2528 | 86.C.-;132.G.T | 0.639565433 | 0.244789313 |
| 12191691 | 2529 | 2.A.-;78.A.-;132.G.T | 0.639118578 | 0.498911309 |
| 8638264 | 2530 | 66.CT.-G;80.A.- | 0.638943302 | 0.281775101 |
| 1195928 | 2531 | -15.T.G;1.TA.-- | 0.638864668 | 0.361194556 |
| 1979286 | 2532 | 0.T.C;81.GA.-T | 0.63859349 | 0.548201787 |
| 8207662 | 2533 | 88.G.-;121.C.A | 0.638318686 | 0.120347159 |
| 6460643 | 2534 | 16.-.C;81.G.- | 0.638310296 | 0.572206436 |
| 2686745 | 2535 | 0.T.-;2.A.C;113.A.C | 0.638107876 | 0.276224167 |
| 1045705 | 2536 | -17.C.A;78.A.- | 0.637718862 | 0.261909741 |
| 8600457 | 2537 | 73.A.-;87.-.A | 0.636224444 | 0.454199961 |
| 7948057 | 2538 | 66.CT.-A;76.-.G | 0.636173306 | 0.379844371 |
| 10091271 | 2539 | 19.-.T;73.AT.-C | 0.636047852 | 0.54205078 |
| 442030 | 2540 | -27.C.A;76.-.A | 0.636046349 | 0.591730246 |
| 844891 | 2541 | 2.A.-;-21.C.A | 0.632935206 | 0.622195627 |
| 10516019 | 2542 | 15.-.T;71.-.C | 0.632798013 | 0.533791186 |
| 12016332 | 2543 | 2.A.-;18.C.- | 0.631955982 | 0.463438076 |
| 8073253 | 2544 | 74.-.C;132.G.C | 0.631661253 | 0.355974737 |
| 8357699 | 2545 | 87.-.G;128.T.G | 0.630236239 | 0.334726151 |
| 2684905 | 2546 | 0.T.-;2.A.C;123.A.C | 0.63013769 | 0.30068044 |
| 2684593 | 2547 | 0.T.-;2.A.C;134.G.T | 0.629727119 | 0.25806889 |
| 12149142 | 2548 | 2.A.-;132.G.T | 0.629713317 | 0.481100174 |
| 2881692 | 2549 | 1.-.C;74.-.C | 0.627981095 | 0.530566104 |
| 5590003 | 2550 | 87.-.G;10.T.C | 0.627660496 | 0.470739888 |
| 12123808 | 2551 | 132.G.T;2.A.-;76.G.- | 0.627589046 | 0.327420951 |
| 8212595 | 2552 | 86.-.C;126.C.A | 0.627387867 | 0.514472305 |
| 8173470 | 2553 | 77.GA.--;121.C.A | 0.626575942 | 0.292013291 |
| 8034488 | 2554 | 72.-.C;82.A.- | 0.626551427 | 0.141402238 |
| 2411142 | 2555 | 1.-.A78.-.C | 0.626392306 | 0.400317799 |
| 8096384 | 2556 | 75.-.A;82.A.- | 0.626331195 | 0.4184413 |
| 2723173 | 2557 | 0.T.-;2.A.C;76.-.G;132.G.C | 0.626278728 | 0.31951463 |
| 8118097 | 2558 | 76.GG.-C;128.T.G | 0.625076866 | 0.405168323 |
| 8543409 | 2559 | 75.-.G;91.AA.-G | 0.624970143 | 0.399800368 |
| 14812614 | 2560 | -29.A.C;76.G.-;78.A.T | 0.624719682 | 0.41001969 |
| 6476723 | 2561 | 16.-.C;76.G.-;78.A.T | 0.624048653 | 0.568485562 |
| 8519286 | 2562 | 76.GG.-T;127.T.G | 0.623896278 | 0.239307789 |
| 8501650 | 2563 | 78.AG.-T | 0.623450189 | 0.439968264 |
| 8208050 | 2564 | 88.G.-;133.A.C | 0.623252172 | 0.206345206 |
| 8549499 | 2565 | 75.C.-;131.A.C | 0.622971653 | 0.381498008 |
| 12009703 | 2566 | 2.A.-;17.-.A | 0.62272951 | 0.617146589 |
| 8128850 | 2567 | 75.-.C;123.A.C | 0.622500225 | 0.271537384 |
| 1862825 | 2568 | 0.TT.--;78.-.T | 0.622420716 | 0.588046598 |
| 6368672 | 2569 | 17.-.A;78.-.C | 0.622294539 | 0.60729061 |
| 8519348 | 2570 | 76.GG.-T;128.T.G | 0.622179066 | 0.277414915 |
| 1041692 | 2571 | -17.C.A;76.GG.-C | 0.621568558 | 0.482033714 |
| 8018631 | 2572 | 72.-.A | 0.620704206 | 0.469244558 |
| 8066533 | 2573 | 74.T.-;128.T.G | 0.619394119 | 0.261300111 |
| 8436892 | 2574 | 81.GA.-T;132.G.T | 0.6187912 | 0.153725765 |
| 8636610 | 2575 | 66.CT.-G;89.A.- | 0.617976625 | 0.523674002 |
| 2884910 | 2576 | 1.-.C;77.-.C | 0.617324835 | 0.494013201 |
| 8143053 | 2577 | 76.G.-;129.C.T | 0.617246947 | 0.285046334 |
| 8356385 | 2578 | 87.-.G;115.T.G | 0.616275923 | 0.347649465 |
| 8561418 | 2579 | 74.-.T;87.-.T | 0.616099222 | 0.531230795 |
| 6467416 | 2580 | 16.-.C;99.-.G | 0.614592516 | 0.506581659 |
| 2723199 | 2581 | 0.T.-;2.A.C;76.-.G132.G.T | 0.614591974 | 0.388667098 |
| 13746674 | 2582 | -13.G.T;75.-.C | 0.614408274 | 0.31688527 |
| 15736191 | 2583 | -32.G.T;76.G.- | 0.613525442 | 0.181348798 |
| 2950619 | 2584 | 1.TA.--;17.T.C | 0.612573777 | 0.330320805 |
| 1250048 | 2585 | -15.T.G;87.-.G | 0.612309332 | 0.301352125 |
| 8519441 | 2586 | 76.GG.-T;130.T.G | 0.611111182 | 0.22661563 |
| 8174044 | 2587 | 77.GA.--;131.A.C | 0.610717722 | 0.367883539 |
| 8083913 | 2588 | 74.-.G;126.C.A | 0.610464009 | 0.361277358 |
| 6554290 | 2589 | 18.C.A;75.-.C | 0.610353714 | 0.248319065 |
| 8481228 | 2590 | 78.A.-;122.A.C | 0.610254061 | 0.293301542 |
| 14004700 | 2591 | -19.G.T;0.T.-;2.A.C | 0.609843143 | 0.268233428 |
| 481605 | 2592 | -27.C.A;2.A.- | 0.609754574 | 0.487237879 |
| 2262447 | 2593 | 0.T.-;81.GA.-C | 0.608367109 | 0.518060275 |
| 2683891 | 2594 | 0.T.-;2.A.C;124.T.G | 0.608299233 | 0.300466966 |
| 2685505 | 2595 | 0.T.-;2.A.C;120.C.T | 0.608011273 | 0.287147596 |
| 827692 | 2596 | -21.C.A;75.-.C | 0.607793108 | 0.315024918 |
| 13101663 | 2597 | -1.GT.--;74.-.T | 0.607364457 | 0.271699421 |
| 2271017 | 2598 | 0.T.-;128.T.G | 0.606729725 | 0.344765189 |
| 8066699 | 2599 | 74.T.-;133.A.C | 0.606568555 | 0.229285806 |
| 8118193 | 2600 | 76.GG.-C;130.T.G | 0.606502407 | 0.534475385 |
| 8073290 | 2601 | 74.-.C;132.G.T | 0.606200531 | 0.307476047 |
| 1117646 | 2602 | -16.C.A;75.-.G | 0.60596891 | 0.417438742 |
| 444910 | 2603 | -27.C.A;86.C.- | 0.604808061 | 0.1069721 |
| 8563682 | 2604 | 75.CG.-T;115.T.G | 0.604638581 | 0.20973375 |
| 14645196 | 2605 | -29.A.C;0.T.-;2.A.C;77.GA.-- | 0.604366944 | 0.450675558 |
| 14663089 | 2606 | -29.A.C;0.T.-;2.A.G;76.-.G | 0.604210237 | 0.579091661 |
| 8480843 | 2607 | 78.A.-;131.A.C;133.A.C | 0.602956995 | 0.220786526 |
| 15241063 | 2608 | -29.A.G;2.A.-;76.-.G | 0.602866438 | 0.535046196 |
| 8128359 | 2609 | 75.-.C;127.T.G | 0.60265641 | 0.24558453 |
| 12202830 | 2610 | 2.A.-;75.-.G;131.A.C | 0.6021552 | 0.300307984 |
| 2516661 | 2611 | 1.T.C;76.-.G | 0.601658638 | 0.569136768 |
| 8600854 | 2612 | 73.A.-;98.-.A | 0.601410904 | 0.554678943 |
| 15158807 | 2613 | -29.A.G;73.-.A | 0.600152864 | 0.594433328 |
| 12147720 | 2614 | 2.A.-;120.C.A | 0.600140012 | 0.523644495 |
| 14344554 | 2615 | -25.A.C;76.GG.-A | 0.599996463 | 0.212388649 |
| 3133295 | 2616 | 1.T.G;3.C.-;74.T.- | 0.599817227 | 0.540582624 |
| 3601058 | 2617 | 2.-.A;76.GG.-T | 0.599399219 | 0.520337615 |
| 8562045 | 2618 | 74.-.T;82.AA.-T | 0.59910687 | 0.25652345 |
| 8080686 | 2619 | 74.-.G;89.-.A | 0.599083728 | 0.541504936 |
| 8116266 | 2620 | 76.GG.-C;115.T.G | 0.599077745 | 0.438717053 |
| 8528148 | 2621 | 76.-.T;86.C.- | 0.597986897 | 0.267868788 |
| 14809572 | 2622 | -29.A.C;82.AA.-T | 0.597370752 | 0.168815452 |
| 1041548 | 2623 | -17.C.A;76.GG.-A | 0.597127645 | 0.347987184 |
| 13847372 | 2624 | -14.A.C;86.-.C | 0.597092285 | 0.439947956 |
| 2654872 | 2625 | 0.T.-;2.A.C;75.C.A | 0.596011018 | 0.360937483 |
| 8543705 | 2626 | 75.-.G;89.A.G | 0.595783213 | 0.480599849 |
| 8150315 | 2627 | 77.-.A;131.A.C | 0.59518379 | 0.216809566 |
| 13854171 | 2628 | -14.A.C;74.-.T | 0.59491988 | 0.255047542 |
| 8084187 | 2629 | 74.-.G;132.G.T | 0.594518766 | 0.378253331 |
| 1249988 | 2630 | -15.T.G;86.C.- | 0.594456707 | 0.263547148 |
| 10308807 | 2631 | 17.-.T;78.A.-;80.A.- | 0.593350924 | 0.537958354 |
| 8093276 | 2632 | 75.-.A;130.T.G | 0.593146278 | 0.294496621 |
| 15069677 | 2633 | -29.A.G;0.T.-;2.A.G;75.-.G | 0.5926846 | 0.429138172 |
| 2884699 | 2634 | 1.-.C;77.-.A | 0.592681567 | 0.444413531 |
| 14921605 | 2635 | -29.A.C;2.A.-;74.-.T | 0.591983792 | 0.536395035 |
| 8448153 | 2636 | 80.A.-;132.G.C | 0.591660429 | 0.174714397 |
| 8140966 | 2637 | 76.G.-;118.T.C | 0.591028328 | 0.208755316 |
| 8161100 | 2638 | 79.6.-;132.G.C | 0.590790681 | 0.220833117 |
| 15165008 | 2639 | -29.A.G;88.-.T | 0.58999307 | 0.294162942 |
| 15058006 | 2640 | -29.A.G;0.T.-;2.A.C;76.GG.-A | 0.589688255 | 0.449116705 |
| 14647360 | 2641 | -29.A.C;0.T.-;2.A.C;75.CG.-T | 0.588777864 | 0.365024825 |
| 8207961 | 2642 | 88.G.-;129.C.A | 0.588244428 | 0.254294724 |
| 2684707 | 2643 | 0.T.-;2.A.C;129.C.G | 0.58718304 | 0.249024882 |
| 12177699 | 2644 | 2.A.-;82.A.-;84.A.T | 0.58696641 | 0.577956828 |
| 8495115 | 2645 | 76.-.G;80.A.G | 0.586627596 | 0.276894747 |
| 8173741 | 2646 | 77.GA.--;126.C.A | 0.585562165 | 0.261884393 |
| 8044380 | 2647 | 72.-.G;87.-.G | 0.585537507 | 0.496438628 |
| 2270366 | 2648 | 0.T.-;120.C.A | 0.585051153 | 0.348301546 |
| 15456767 | 2649 | -30.C.G;74.-.T | 0.584964692 | 0.259355294 |
| 12752882 | 2650 | 0.-.T;73.AT.-G | 0.583581773 | 0.561012988 |
| 4217308 | 2651 | 4.T.-;71.T.C | 0.583528708 | 0.515253098 |
| 14810890 | 2652 | -29.A.C;78.AG.-C | 0.583180403 | 0.367641912 |
| 13853442 | 2653 | -14.A.C;76.GG.-T | 0.582589545 | 0.211217084 |
| 8448176 | 2654 | 80.A.- | 0.582531333 | 0.209077508 |
| 8103057 | 2655 | 76.GG.-A;98.-.A | 0.582277673 | 0.55389364 |
| 8141130 | 2656 | 76.G.-;118.T.G | 0.581284111 | 0.26198905 |
| 8133120 | 2657 | 75.-.C;86.-.G | 0.581268194 | 0.268509352 |
| 14921140 | 2658 | -29.A.C;2.A.-;76.-.G | 0.581166066 | 0.463527496 |
| 1046627 | 2659 | -17.C.A;74.-.T | 0.580843268 | 0.237913321 |
| 8490817 | 2660 | 76.-.G;122.A.C | 0.580816128 | 0.338035457 |
| 2749021 | 2661 | 0.T.-;2.A.C;65.G.T | 0.580627515 | 0.520199907 |
| 1251730 | 2662 | -15.T.G;78.-.0 | 0.580454498 | 0.277680214 |
| 8565400 | 2663 | 75.CG.-T;131.AG.CC | 0.580378421 | 0.162900123 |
| 8034315 | 2664 | 72.-.C;87.-.G | 0.579900852 | 0.400196584 |
| 1095467 | 2665 | -16.C.A;0.T.-;2.A.C | 0.578139753 | 0.253542538 |
| 1982142 | 2666 | 0.T.C;70.-.T | 0.578040747 | 0.514803955 |

TABLE 23

| | SEQ | | | |
|---|---|---|---|---|
| index | ID NO | muts_lindexed | MI | 95% CI |
| 2661968 | 2667 | 0.T.-;2.A.C;76.G.-;133.A.C | 0.57749224 | 0.441653169 |
| 14529775 | 2668 | -28.G.T;75.-.G | 0.577078051 | 0.357956174 |
| 2464540 | 2669 | 0.T.-;3.C.-;82.AA.-- | 0.576438266 | 0.496783332 |
| 3011533 | 2670 | 1.TA.--;126.C.A | 0.576212191 | 0.385876942 |
| 8160673 | 2671 | 79.G.-;121.C.A | 0.576161715 | 0.276769402 |
| 445036 | 2672 | -27.C.A;87.-.T | 0.576139586 | 0.385762845 |
| 8480668 | 2673 | 78.A.-;130.T.C | 0.576024382 | 0.239310768 |
| 446329 | 2674 | -27.C.A;78.-.C | 0.575818594 | 0.275614681 |
| 8524684 | 2675 | 76.-.T;86.-.C | 0.575418001 | 0.427849393 |
| 14350148 | 2676 | -25.A.C;78.A.- | 0.574994909 | 0.251987218 |
| 15456629 | 2677 | -30.C.G;75.C.- | 0.574735978 | 0.433262652 |
| 8084175 | 2678 | 74.-.G;133.A.C | 0.573978066 | 0.497590865 |
| 8470281 | 2679 | 78.-.C;133.A.C | 0.573588021 | 0.327243841 |
| 1976159 | 2680 | 0.T.C;88.G.- | 0.573415984 | 0.487091048 |
| 2553815 | 2681 | 0.T.-;2.A.C;11.T.C | 0.572813487 | 0.380949243 |
| 8565313 | 2682 | 75.CG.-T;130.T.G | 0.572720854 | 0.28519884 |
| 8142626 | 2683 | 76.G.-;128.T.C | 0.572573376 | 0.270734577 |
| 15059444 | 2684 | -29.A.G;0.T.-;2.A.C;76.GG.-T | 0.571014973 | 0.539165235 |
| 14349990 | 2685 | -25.A.C;78.-.C | 0.570479705 | 0.339570631 |
| 7944404 | 2686 | 66.CT.-A;86.-.C | 0.570401891 | 0.517202925 |
| 8143508 | 2687 | 76.G.-;122.A.G | 0.570368433 | 0.295091218 |
| 8483736 | 2688 | 78.A.-;99.-.G | 0.569940382 | 0.383399129 |
| 8457128 | 2689 | 80.AG.-T | 0.569875532 | 0.407717978 |
| 14685680 | 2690 | -29.A.C;4.T.-;76.GG.-C | 0.569769951 | 0.468156843 |
| 8639135 | 2691 | 66.CT.-G;75.-.G | 0.569640144 | 0.439103296 |
| 8093196 | 2692 | 75.-.A;128.T.G | 0.569631485 | 0.286483725 |
| 2574670 | 2693 | 0.T.-2.A.C;21.T.A | 0.568848291 | 0.277790817 |
| 2270511 | 2694 | 0.T.-;121.C.A | 0.568823446 | 0.346919825 |
| 2411434 | 2695 | 1.-.A;78.A.- | 0.568308397 | 0.492015937 |
| 8128649 | 2696 | 75.-.C;131.A.C;133.A.C | 0.56797398 | 0.310988199 |
| 2837903 | 2697 | 2.A.C;0.T.-;5.G.T | 0.567182668 | 0.301762792 |
| 15456872 | 2698 | -30.C.G;75.CG.-T | 0.566922487 | 0.275000232 |
| 2684575 | 2699 | 130.--T.TAG;133.A.G;2.A.C;0.T.- | 0.566786287 | 0.297282581 |
| 15486653 | 2700 | -30.C.G;2.A.- | 0.566597124 | 0.457183039 |
| 12202811 | 2701 | 2.A.-;75.-.G;133.A.C | 0.565986807 | 0.395655607 |
| 8480879 | 2702 | 78.A.-;129.C.G | 0.565951849 | 0.323772129 |
| 3011188 | 2703 | 1.TA.--;121.C.A | 0.563547027 | 0.371989823 |
| 8297879 | 2704 | 99.-.G | 0.563426918 | 0.267608562 |
| 8352639 | 2705 | 86.C.-;127.T.G | 0.563082098 | 0.202268903 |
| 14801514 | 2706 | -29.A.C;86.-.A | 0.562277455 | 0.47388314 |
| 1975537 | 2707 | 0.T.C;79.G.- | 0.562276863 | 0.48611243 |
| 8480783 | 2708 | 78.A.-;134.G.T | 0.560674716 | 0.40924491 |
| 14351204 | 2709 | -25.A.C;75.C.- | 0.56061618 | 0.404146443 |
| 1042672 | 2710 | -17.C.A;87.-.A | 0.560291693 | 0.386629447 |
| 8480385 | 2711 | 78.A.-;126.C.A | 0.56011981 | 0.238382308 |
| 8105496 | 2712 | 76.GG.-A;127.T.G | 0.559463981 | 0.268526426 |
| 15059173 | 2713 | -29.A.G;0.T.-;2.A.C;80.A.- | 0.558328951 | 0.364430265 |
| 8132470 | 2714 | 75.-.C;91.AA.-G | 0.55794057 | 0.467738717 |
| 14663399 | 2715 | -29.A.C;0.T.-;2.A.G;75.C.- | 0.555989953 | 0.452975089 |
| 8132353 | 2716 | 75.-.C;91.A.-;93.A.G | 0.555655149 | 0.391589733 |
| 6557204 | 2717 | 18.C.A;78.A.- | 0.55490577 | 0.33009122 |
| 13845080 | 2718 | -14.A.C;75.-.A | 0.553964545 | 0.280917125 |
| 2894429 | 2719 | 1.-.C;86.-.G | 0.553556726 | 0.355589983 |
| 8605594 | 2720 | 73.A.-;87.-.T | 0.553338911 | 0.323431172 |
| 14918668 | 2721 | -29.A.C;2.A.-;75.-.A | 0.553238993 | 0.285233158 |
| 13852859 | 2722 | -14.A.C;76.-.G | 0.552869618 | 0.304031476 |
| 8558273 | 2723 | 74.-.T;126.C.A | 0.552629697 | 0.203156607 |
| 14344734 | 2724 | -25.A.C;76.GG.-C | 0.552119262 | 0.424653466 |
| 8063226 | 2725 | 74.T.-;87.-.A | 0.552096685 | 0.354902882 |
| 8564564 | 2726 | 75.CG.-T;119.C.A | 0.551864161 | 0.230129505 |
| 13687669 | 2727 | -12.G.T75.-.G | 0.551148172 | 0.378236607 |
| 14812439 | 2728 | -29.A.C;78.A.T | 0.550882224 | 0.501507682 |
| 7944045 | 2729 | 66.CT.-A;76.G.- | 0.550594074 | 0.425751575 |
| 2685752 | 2730 | 0.T.-;2.A.C;119.C.T | 0.549480674 | 0.2058528 |
| 8118242 | 2731 | 130.--T.TAG;133.A.G;76.GG.-C | 0.548710279 | 0.423160468 |
| 1245577 | 2732 | -15.T.G;73.-.A | 0.548630123 | 0.53908022 |
| 15454032 | 2733 | -30.C.G;86.C.- | 0.548408194 | 0.146894103 |
| 15738375 | 2734 | -32.G.T;75.-.G | 0.548196327 | 0.30032935 |
| 6302341 | 2735 | 16.-.A;72.-.C | 0.54793736 | 0.363280011 |
| 2287278 | 2736 | 0.T.-;82.-.T | 0.547862516 | 0.435436106 |
| 3599083 | 2737 | 2.-.A;78.-.C | 0.547517977 | 0.397685932 |
| 8538303 | 2738 | 75.-.G;129.C.G | 0.547177668 | 0.446183912 |
| 3025181 | 2739 | 1.TA.--;82.-.T | 0.546005635 | 0.497627964 |
| 999582 | 2740 | -17.C.A;0.T.- | 0.545876413 | 0.406976245 |
| 9986114 | 2741 | 19.-.G;89.-.C | 0.545714579 | 0.49212709 |
| 13096860 | 2742 | -1.GT.--;74.T.- | 0.54540182 | 0.126101418 |
| 14686894 | 2743 | -29.A.C;4.T.-;86.C.- | 0.545239171 | 0.409735305 |
| 8515608 | 2744 | 76.G.-;78.AG.TT | 0.545069364 | 0.313301484 |
| 10071761 | 2745 | 19.-.T;85.TC.-A | 0.54479944 | 0.527860057 |
| 8540169 | 2746 | 75.-.G;113.A.G | 0.543102637 | 0.381475433 |
| 15170520 | 2747 | -29.A.G;73.AT.-G | 0.542963315 | 0.302212358 |
| 8133499 | 2748 | 75.-.C;83.-.G | 0.542495998 | 0.398113706 |
| 15161304 | 2749 | -29.A.G;76.G.-;78.A.C | 0.542401586 | 0.360524231 |
| 14815543 | 2750 | -29.A.C;73.AT.-G | 0.542111484 | 0.268698449 |
| 14812304 | 2751 | -29.A.C;78.-.T | 0.541883351 | 0.456256042 |
| 8351219 | 2752 | 86.C.-;115.T.G | 0.541795444 | 0.167333867 |
| 8363173 | 2753 | 87.-.T;129.C.A | 0.541710882 | 0.45548051 |
| 8128504 | 2754 | 75.-.C;130.T.C | 0.541636404 | 0.301115914 |
| 8538167 | 2755 | 75.-.G;132.GA.CC | 0.541089363 | 0.415736007 |
| 8063302 | 2756 | 74.T.-;88.G.- | 0.540731374 | 0.306571561 |
| 10087552 | 2757 | 19.-.T;78.A.-;80.A.- | 0.540592506 | 0.495589309 |
| 7490687 | 2758 | 36.C.A;76.G.- | 0.540151999 | 0.152783677 |
| 8202465 | 2759 | 87.-.A;132.G.T | 0.54005277 | 0.527499683 |
| 8519530 | 2760 | 76.GG.-T;131.AG.CC | 0.539568972 | 0.199248804 |
| 4321391 | 2761 | 4.T.-;65.G.T | 0.538942702 | 0.513208936 |
| 15239627 | 2762 | -29.A.G;2.A.-;75.-.C | 0.538937683 | 0.394383352 |
| 14808642 | 2763 | -29.A.C;82.A.-;84.A.T | 0.538835503 | 0.494127547 |
| 12123800 | 2764 | 2.A.-;76.G.-;133.A.C | 0.53867639 | 0.36512328 |
| 15169507 | 2765 | -29.A.G;75.C.- | 0.538649298 | 0.410436551 |
| 2731526 | 2766 | 0.T.-;2.A.C;75.-.G;132.G.T | 0.538312596 | 0.51810426 |
| 8118032 | 2767 | 76.GG.-C;127.T.G | 0.53700376 | 0.351634793 |
| 15168665 | 2768 | -29.A.G;77.-.T | 0.536694116 | 0.500951198 |
| 8546114 | 2769 | 75.C.-;88.G.- | 0.536531987 | 0.433499049 |
| 6480287 | 2770 | 16.-.C;73.A.G | 0.535878646 | 0.477206798 |
| 8367284 | 2771 | 86.-.G;121.C.A | 0.535296368 | 0.178941915 |
| 14245829 | 2772 | -24.G.T;78.A.- | 0.534877866 | 0.289282764 |
| 8526256 | 2773 | 76.-.T;121.C.A | 0.534562327 | 0.258036007 |
| 320895 | 2774 | -28.G.C;75.-.G | 0.533966141 | 0.338633053 |
| 14801003 | 2775 | -29.A.C;85.TC.-A | 0.533852209 | 0.42681567 |
| 2900348 | 2776 | 1.-.C;76.G.-;78.A.T | 0.533722522 | 0.476159074 |
| 8173897 | 2777 | 77.GA.--;129.C.A | 0.533268703 | 0.286973833 |
| 10315449 | 2778 | 17.-.T;73.A.G | 0.532731562 | 0.462080339 |
| 8118283 | 2779 | 76.GG.-C;131.AG.CC | 0.532401677 | 0.506645788 |
| 8638120 | 2780 | 66.CT.-G;81.GA.-T | 0.529612827 | 0.189572957 |
| 8115215 | 2781 | 76.GG.-C;98.-.A | 0.529601406 | 0.407199505 |
| 8098639 | 2782 | 75.CG.-A | 0.528065372 | 0.398201351 |
| 8363276 | 2783 | 87.-.T;133.A.C | 0.527654337 | 0.444969797 |
| 8490333 | 2784 | 76.-.G;130.T.G | 0.527134113 | 0.344258636 |
| 670332 | 2785 | -23.C.A;76.G.- | 0.526515155 | 0.335457235 |
| 14499641 | 2786 | -28.G.T;0.T.-;2.A.C | 0.52630839 | 0.192014079 |
| 8357643 | 2787 | 87.-.G;127.T.G | 0.526215994 | 0.313357684 |
| 4269759 | 2788 | 4.T.-;91.A.-;93.A.G | 0.526142398 | 0.366589265 |
| 8145628 | 2789 | 76.G.-;113.A.G | 0.525564142 | 0.316731543 |
| 1250181 | 2790 | -15.T.G;86.-.G | 0.525481067 | 0.170826111 |
| 2684458 | 2791 | 0.T.-;2.A.C;130.T.C | 0.524709128 | 0.229934214 |
| 8211364 | 2792 | 86.-.C;115.T.G | 0.524286326 | 0.484460897 |
| 12327615 | 2793 | 2.A.-;6.G.T | 0.523903903 | 0.498314675 |
| 13750639 | 2794 | -13.G.T;76.GG.-T | 0.52360612 | 0.199695415 |
| 8545256 | 2795 | 75.-.G;82.AA.-T | 0.523533206 | 0.310507673 |
| 15051403 | 2796 | -29.A.G;0.T.-;76.G.- | 0.523477863 | 0.359359453 |
| 8128996 | 2797 | 75.-.C;122.A.C | 0.52294617 | 0.295511794 |
| 15157689 | 2798 | -29.A.G;72.-.A | 0.522828828 | 0.3905261 |
| 3011885 | 2799 | 1.TA.--;131.A.C | 0.522211145 | 0.412727331 |
| 6586124 | 2800 | 18.-.A;73.AT.-C | 0.521721358 | 0.392610894 |
| 8538269 | 2801 | 75.-.G;131.A.G | 0.521700337 | 0.380171958 |
| 2661660 | 2802 | 0.T.-;2.A.C;76.G.-;121.C.A | 0.52050173 | 0.428916241 |
| 8490491 | 2803 | 76.-.G;131.A.G | 0.520366526 | 0.267501834 |
| 8638542 | 2804 | 66.CT.-G;78.-.C | 0.519761904 | 0.367445975 |
| 14230312 | 2805 | -24.G.T;0.T.-;2.A.C | 0.519671019 | 0.345673439 |
| 6554102 | 2806 | 18.C.A;76.GG.-A | 0.519352035 | 0.207450089 |
| 8480490 | 2807 | 78.A.-;127.T.G | 0.519219321 | 0.21628878 |
| 12148735 | 2808 | 2.A.-;127.T.G | 0.518903576 | 0.454392832 |
| 6554952 | 2809 | 18.C.A;86.-.C | 0.518790459 | 0.411420745 |
| 8548546 | 2810 | 75.C.-;119.C.A | 0.517924262 | 0.375435555 |
| 8537738 | 2811 | 75.-.G;125.T.G | 0.517546384 | 0.421774082 |
| 14524986 | 2812 | -28.G.T;76.G.- | 0.517443138 | 0.210817034 |
| 8112028 | 2813 | 76.-.A;121.C.A | 0.517164085 | 0.479428413 |
| 8558469 | 2814 | 74.-.T;130.T.G | 0.517109614 | 0.240257462 |
| 8536730 | 2815 | 75.-.G;118.T.G | 0.516654079 | 0.347346716 |
| 1975405 | 2816 | 0.T.C;77.-.A | 0.516223556 | 0.381140846 |
| 8490677 | 2817 | 76.-.6;123.A.C | 0.515655644 | 0.354670318 |
| 14351455 | 2818 | -25.A.C;75.CG.-T | 0.515062617 | 0.304205957 |
| 8519708 | 2819 | 76.GG.-T;123.A.C | 0.514732027 | 0.221694148 |
| 13850181 | 2820 | -14.A.C;86.C.- | 0.514653567 | 0.175135516 |
| 829963 | 2821 | -21.C.A;76.GG.-T | 0.512665825 | 0.195077868 |
| 396157 | 2822 | -27.C.A;1.TA.-- | 0.512397621 | 0.411313736 |
| 8128583 | 2823 | 130.--T.TAG;133.A.G;75.-.C | 0.511360625 | 0.326791328 |
| 3011846 | 2824 | 1.TA.--;133.A.C | 0.510597585 | 0.351631622 |
| 14918900 | 2825 | -29.A.C;2.A.-;75.-.C | 0.510304993 | 0.475271006 |
| 15159253 | 2826 | -29.A.G;74.-.C | 0.509144831 | 0.438279977 |
| 8480820 | 2827 | 78.A.-;131.AG.CC | 0.508771663 | 0.277308284 |
| 2824789 | 2828 | 0.T.-;2.A.C;16.C.- | 0.508408045 | 0.431164458 |
| 8030574 | 2829 | 72.-.C;88.G.- | 0.506884465 | 0.293464717 |

TABLE 24

| | SEQ | | | |
|---|---|---|---|---|
| index | ID NO | muts_lindexed | MI | 95% CI |
| 8103971 | 2830 | 76.GG.-A;115.T.G | 0.506714342 | 0.334208414 |
| 8480769 | 2831 | 130.--T.TAG;133.A.G;78.A.- | 0.506662335 | 0.275750543 |
| 12146846 | 2832 | 2.A.-;118.T.C | 0.506662335 | 0.448261871 |
| 8105632 | 2833 | 76.GG.-A;130.T.G | 0.506661965 | 0.31757799 |
| 14655186 | 2834 | -29.A.C;1.TA.--;78.A.- | 0.505038768 | 0.349546779 |
| 13887801 | 2835 | -14.A.C;2.A.- | 0.50476973 | 0.416608677 |
| 8558448 | 2836 | 74.-.T;130.T.C | 0.504326742 | 0.274992635 |
| 8588552 | 2837 | 73.AT.-G;87.-.G | 0.503452084 | 0.382877256 |
| 4277297 | 2838 | 4.T.-;86.C.T | 0.50273009 | 0.316942926 |
| 8490414 | 2839 | 130.--T.TAG;133.A.G;76.-.G | 0.502294014 | 0.265692536 |
| 8557082 | 2840 | 74.-.T;115.T.G | 0.501788618 | 0.240258884 |
| 3010886 | 2841 | 1.TA.--;119.C.A | 0.501621564 | 0.332438342 |
| 8123134 | 2842 | 75.-.C;82.-.A | 0.500644531 | 0.401625156 |
| 8558564 | 2843 | 74.-.T;131.AG.CC | 0.500523453 | 0.241207919 |
| 10570905 | 2844 | 15.-.T;66.C.- | 0.500493846 | 0.475165652 |
| 8448232 | 2845 | 80.A.-;131.A.C | 0.499354119 | 0.207066339 |
| 1041390 | 2846 | -17.C.A;75.-.A | 0.499154073 | 0.323859893 |
| 646656 | 2847 | -23.C.A;0.T.-;2.A.C | 0.499025819 | 0.25793286 |
| 15167125 | 2848 | -29.A.G;80.A.- | 0.498690448 | 0.246341392 |
| 8105551 | 2849 | 76.GG.-A;128.T.G | 0.497708543 | 0.268069258 |
| 8084057 | 2850 | 74.-.G;129.C.A | 0.495342021 | 0.351272002 |
| 8493858 | 2851 | 76.-.G;91.A.- | 0.495092834 | 0.442273746 |
| 10544166 | 2852 | 15.-.T;91.A.-;93.A.G | 0.494903344 | 0.36111403 |
| 8565224 | 2853 | 75.CG.-T;128.T.G | 0.493977822 | 0.257917935 |
| 8586274 | 2854 | 73.AT.-G;131.A.C | 0.493739387 | 0.325651011 |
| 8362865 | 2855 | 87.-.T;121.C.A | 0.493526779 | 0.439303415 |
| 443254 | 2856 | -27.C.A;88.G.- | 0.492968287 | 0.160647841 |
| 13171639 | 2857 | -1.G.T;75.-.G | 0.492601142 | 0.491746074 |
| 8478628 | 2858 | 78.A.-;116.T.G | 0.491876176 | 0.261017897 |
| 6557301 | 2859 | 18.C.A;76.-.G | 0.49164967 | 0.407268607 |
| 8752532 | 2860 | 55.-.T;75.-.A | 0.491390512 | 0.44462484 |
| 8560929 | 2861 | 74.-.T;91.A.-;93.A.G | 0.491205156 | 0.384453162 |
| 4295718 | 2862 | 4.T.-;78.A.-;132.G.C | 0.491177117 | 0.428226189 |
| 10561864 | 2863 | 15.-.T;76.G.T | 0.491146433 | 0.343126473 |
| 8537677 | 2864 | 75.-.G;125.T.C | 0.489714365 | 0.274407052 |
| 8143025 | 2865 | 76.G.-;129.C.G | 0.489227868 | 0.327699958 |
| 8089936 | 2866 | 75.-.A;89.-.A | 0.488779674 | 0.372660333 |
| 8599794 | 2867 | 70.-.T;76.-.G | 0.488667386 | 0.391145449 |
| 8105873 | 2868 | 76.GG.-A;123.A.C | 0.487861644 | 0.22247771 |
| 8517616 | 2869 | 76.GG.-T;115.T.G | 0.486978242 | 0.198126193 |
| 12149710 | 2870 | 2.A.-;122.A.C | 0.485932471 | 0.444772033 |
| 8489904 | 2871 | 76.-.G;124.T.G | 0.485539102 | 0.229906368 |
| 1164547 | 2872 | -15.T.C;76.G.- | 0.485109654 | 0.30382645 |
| 8653886 | 2873 | 65.GC.-T;87.-.6 | 0.485040713 | 0.238958896 |
| 8074762 | 2874 | 74.-.C;86.C.- | 0.484897947 | 0.341794685 |
| 8480183 | 2875 | 78.A.-;124.T.G | 0.484866253 | 0.155741545 |
| 14921899 | 2876 | -29.A.C;2.A.-;73.A.- | 0.484654008 | 0.412332886 |
| 806417 | 2877 | -21.C.A;0.T.-;2.A.C | 0.484651885 | 0.213811885 |
| 8367608 | 2878 | 86.-.G;132.G.T | 0.484324949 | 0.200140872 |
| 3000591 | 2879 | 1.TA.--;76.G.-;132.G.C | 0.4836883 | 0.410892791 |
| 8602683 | 2880 | 73.A.-;121.C.A | 0.48312272 | 0.181092975 |
| 1250113 | 2881 | -15.T.G;87.-.T | 0.482791984 | 0.353024933 |
| 1246020 | 2882 | -15.T.G;74.-.G | 0.482594805 | 0.468388077 |
| 8095244 | 2883 | 75.-.A;99.-.G | 0.482411376 | 0.440951749 |
| 7516650 | 2884 | 38.C.A;75.-.G | 0.482411376 | 0.23182513 |
| 8101468 | 2885 | 75.C.A;78.A.- | 0.482082335 | 0.243384018 |
| 6420798 | 2886 | 17.T.C;76.G.- | 0.481444121 | 0.122802281 |
| 8080536 | 2887 | 74.-.G;88.G.- | 0.481189232 | 0.304120518 |
| 8583631 | 2888 | 73.AT.-G;86.-.C | 0.481173989 | 0.328294793 |
| 2685339 | 2889 | 0.T.-;2.A.C;121.C.T | 0.480161236 | 0.259384948 |
| 15241190 | 2890 | -29.A.G;2.A.-;76.3G.-T | 0.480084038 | 0.448042386 |
| 4235216 | 2891 | 4.T.-;77.G.A | 0.479539261 | 0.358264062 |
| 333335 | 2892 | 2.A.-;-28.G.C | 0.479358813 | 0.436521088 |
| 15454091 | 2893 | -30.C.G;87.-.G | 0.479044667 | 0.245281612 |
| 8104903 | 2894 | 76.GG.-A;119.C.A | 0.478218223 | 0.290640621 |
| 14795119 | 2895 | -29.A.C72.-.C | 0.478167361 | 0.366311838 |
| 8549156 | 2896 | 126.C.A;75.C.- | 0.477655337 | 0.401183875 |
| 2270186 | 2897 | 0.T.-;119.C.A | 0.476357464 | 0.28961569 |
| 442714 | 2898 | -27.C.A;79.G.- | 0.475921463 | 0.33589485 |
| 2684191 | 2899 | 0.T.-;2.A.C;127.T.C | 0.475552623 | 0.230755681 |
| 2661980 | 2900 | 0.T.-;2.A.C;76.G.-;132.G.T | 0.475543203 | 0.461390486 |
| 8759441 | 2901 | 55.-.T;75.CG.-T | 0.475274664 | 0.3110126 |
| 8548730 | 2902 | 75.C.-;120.C.A | 0.474785619 | 0.390058461 |
| 2517486 | 2903 | 1.T.C;75.CG.-T | 0.474646379 | 0.383115501 |
| 13098412 | 2904 | -1.GT.--;86.-.C | 0.473674402 | 0.202438358 |
| 6556251 | 2905 | 18.C.A;87.-.G | 0.471145708 | 0.219704096 |
| 8539383 | 2906 | 75.-.G;117.G.T | 0.470019299 | 0.350569819 |
| 2728409 | 2907 | 0.T.-;2.A.C;76.GG.-T;132.G.T | 0.469423673 | 0.457772037 |
| 8147743 | 2908 | 76.G.-;89.-.C | 0.468585571 | 0.171258383 |
| 8538151 | 2909 | 75.-.G;132.G.A | 0.467133266 | 0.349055208 |
| 8519808 | 2910 | 76.GG.-T;122.A.C | 0.466576243 | 0.178702651 |
| 8538739 | 2911 | 75.-.G;122.A.G | 0.466576243 | 0.334549602 |
| 8055399 | 2912 | 73.-.A;88.G.- | 0.466033327 | 0.320041272 |
| 8602922 | 2913 | 73.A.-;126.C.A | 0.465865335 | 0.283031316 |
| 8558390 | 2914 | 74.-.T;128.T.G | 0.46527251 | 0.205871798 |
| 8202371 | 2915 | 87.-.A;129.C.A | 0.465267382 | 0.464757478 |
| 8495023 | 2916 | 78.A.-;82.A.G | 0.463214654 | 0.211642756 |
| 8093252 | 2917 | 75.-.A;130.T.C | 0.463013832 | 0.334659591 |
| 2566367 | 2918 | 0.T.-2.A.C;17.T.C | 0.461392589 | 0.268420878 |
| 443194 | 2919 | -27.C.A;87.-.A | 0.460771587 | 0.399261729 |
| 8586216 | 2920 | 73.AT.-G;132.G.C | 0.460668725 | 0.250991995 |
| 8492129 | 2921 | 76.-.G;113.A.G | 0.459948539 | 0.273948034 |
| 8602593 | 2922 | 73.A.-;120.C.A | 0.459546198 | 0.167376352 |
| 12438314 | 2923 | 1.TAC.---;76.-.T | 0.458955662 | 0.409257705 |
| 8018666 | 2924 | 72.-A;111.A.C | 0.458702522 | 0.405962971 |
| 2658141 | 2925 | 0.T.-;2.A.C;76.GG.-C;132.G.C | 0.458544612 | 0.41841279 |
| 2270855 | 2926 | 0.T.-;126.C.A | 0.458127918 | 0.339841458 |
| 3011711 | 2927 | 1.TA.--;129.C.A | 0.457672819 | 0.369464206 |
| 8357785 | 2928 | 87.-.G;130.T.G | 0.457390155 | 0.321441502 |
| 12148855 | 2929 | 2.A.-;128.T.G | 0.456649691 | 0.424208993 |
| 8538425 | 2930 | 75.-.G;126.C.T | 0.456066648 | 0.391670844 |
| 14812176 | 2931 | -29.A.C;78.AG.-T | 0.455217768 | 0.421822764 |
| 959345 | 2932 | -18.T.G;0.T.-;2.A.C | 0.454745656 | 0.262947402 |
| 8352569 | 2933 | 86.C.-;126.C.A | 0.451977309 | 0.231744784 |
| 8562579 | 2934 | 75.CG.-T;86.-.C | 0.451863845 | 0.284864192 |
| 12185280 | 2935 | 2.A.-;80.A.-;132.G.C | 0.451858405 | 0.397487978 |
| 8118567 | 2936 | 76.GG.-C;122.A.C | 0.449218148 | 0.341479227 |
| 8129443 | 2937 | 75.-.C;119.C.T | 0.448058984 | 0.241337157 |
| 8488242 | 2938 | 76.-.G;115.T.G | 0.447807737 | 0.303351067 |
| 2685947 | 2939 | 0.T.-;2.A.C;117.G.T | 0.447350974 | 0.223995386 |
| 2684042 | 2940 | 0.T.-;2.A.C;125.T.G | 0.446446953 | 0.225442366 |
| 2628011 | 2941 | 0.T.-;2.A.C;65.G.A | 0.445909737 | 0.431014642 |
| 1093922 | 2942 | -16.C.A;0.T.- | 0.445744275 | 0.384769858 |
| 14021392 | 2943 | -19.G.T;76.G.- | 0.445446692 | 0.210980489 |
| 14023783 | 2944 | -19.G.T;75.-.G | 0.445006163 | 0.320561961 |
| 8479108 | 2945 | 118.T.C;78.A.- | 0.444437185 | 0.180007604 |
| 4295742 | 2946 | 4.T.-;78.A.-;132.G.T | 0.443700313 | 0.342467455 |
| 8348822 | 2947 | 88.-.T;132.G.C | 0.443636958 | 0.306921941 |
| 8448031 | 2948 | 80.A.-;128.T.G | 0.442657435 | 0.216018231 |
| 8480854 | 2949 | 78.A.-;131.A.G | 0.442172304 | 0.339275348 |
| 8073282 | 2950 | 74.-.C;133.A.C | 0.441868617 | 0.352017188 |
| 2271058 | 2951 | 129.C.A;0.T.- | 0.441858081 | 0.316640496 |
| 12151722 | 2952 | 2.A.-;113.A.C | 0.44078825 | 0.348903885 |
| 13168765 | 2953 | -1.G.T;76.G.- | 0.440234903 | 0.237503321 |
| 8760885 | 2954 | 56.G.T;76.G.- | 0.438783025 | 0.163508619 |
| 8518019 | 2955 | 76.GG.-T;116.T.G | 0.438369692 | 0.235604662 |
| 1117245 | 2956 | -16.C.A;78.A.- | 0.438279124 | 0.16834881 |
| 8592769 | 2957 | 70.-.T;88.G.- | 0.438220877 | 0.244749237 |
| 8628663 | 2958 | 66.CT.-G;79.G.- | 0.438072351 | 0.182645901 |
| 8480752 | 2959 | 78.A.-;132.GA.CC | 0.437930513 | 0.248881928 |
| 8059585 | 2960 | 73.-.A;86.C.- | 0.437225419 | 0.435957495 |
| 13750261 | 2961 | -13.G.T;78.A.- | 0.437054685 | 0.253065367 |
| 8539599 | 2962 | 75.-.G;114.G.T | 0.436888965 | 0.374443118 |
| 8352028 | 2963 | 86.C.-;119.C.A | 0.436035802 | 0.188996533 |
| 8129947 | 2964 | 75.-.C;113.A.C | 0.43594687 | 0.304848987 |
| 8538081 | 2965 | 75.-.G;130.T.C;132.G.C | 0.434698024 | 0.332020273 |
| 8561460 | 2966 | 74.-.T;86.-.G | 0.432879878 | 0.233198854 |
| 8363222 | 2967 | 87.-.T;130.T.G | 0.432369032 | 0.345082874 |
| 15749286 | 2968 | -32.G.T;2.A.- | 0.43081932 | 0.390213068 |
| 8129269 | 2969 | 75.-.C;120.C.T | 0.430595045 | 0.273748314 |
| 445858 | 2970 | -27.C.A;82.AA.-T | 0.430559526 | 0.234423079 |
| 8133915 | 2971 | 75.-.C;80.A.G | 0.430504694 | 0.343719431 |
| 1045161 | 2972 | -17.C.A;82.AA.-T | 0.430467643 | 0.182104489 |
| 2569551 | 2973 | 0.T.-;2.A.C;18.C.A | 0.430355335 | 0.27785676 |
| 8034268 | 2974 | 72.-.C;86.C.- | 0.427635605 | 0.226345972 |
| 481315 | 2975 | -27.C.A;2.A.-;76.G.- | 0.427566605 | 0.366076873 |
| 447361 | 2976 | -27.C.A;75.C.- | 0.427271989 | 0.372051561 |
| 393117 | 2977 | -27.C.A;0.T.-;2.A.C;76.G.- | 0.427167737 | 0.380439384 |
| 672550 | 2978 | -23.C.A;76.GG.-T | 0.426979754 | 0.135361911 |
| 13171223 | 2979 | -1.G.T;78.A.- | 0.426700654 | 0.170495659 |
| 2269114 | 2980 | 0.T.-;115.T.G | 0.424407199 | 0.334312683 |
| 15164751 | 2981 | -29.A.G;89.-.C | 0.424272539 | 0.193097014 |
| 8150288 | 2982 | 77.-.A;133.A.C | 0.423804972 | 0.252292931 |
| 13716962 | 2983 | -13.G.T;0.T.-;2.A.C | 0.42315833 | 0.20734707 |
| 14810153 | 2984 | -29.A.C;80.A.- | 0.422936471 | 0.207060587 |
| 8149925 | 2985 | 77.-.A;121.C.A | 0.42217724 | 0.192407441 |
| 8118444 | 2986 | 76.GG.-C;123.A.C | 0.421898172 | 0.264213012 |
| 15450237 | 2987 | -30.C.G;74.T.- | 0.421545908 | 0.305538885 |
| 13847292 | 2988 | -14.A.C;88.G.- | 0.421223502 | 0.122864931 |
| 8599283 | 2989 | 70.-.T;82.AA.-G | 0.42040004 | 0.308617971 |
| 2258810 | 2990 | 0.T.-;76.G.-;132.G.C | 0.420140578 | 0.380686219 |
| 8352862 | 2991 | 86.C.-;131.AG.CC | 0.42006813 | 0.340106853 |
| 8431466 | 2992 | 82.AA.-T;121.C.A | 0.418074771 | 0.20942073 |
| 10604385 | 2993 | 16.C.T;76.GG.-C | 0.418006899 | 0.309663803 |

TABLE 25

| | SEQ | | | |
|---|---|---|---|---|
| index | ID NO | muts_lindexed | MI | 95% CI |
| 15410869 | 2994 | -30.C.G;1.TA.-- | 0.417875135 | 0.3568233 |
| 14644576 | 2995 | -29.A.C;0.T.-;2.A.C;74 | 0.417019277 | 0.397760744 |
| 8174011 | 2996 | 77.GA.--;133.A.C | 0.416289819 | 0.329786398 |
| 13750370 | 2997 | -13.G.T;76.-.G | 0.415803975 | 0.250075934 |
| 8083409 | 2998 | 74.-.G;119.C.A | 0.415582401 | 0.37566693 |
| 8093325 | 2999 | 130.--T.TAG;133.A.G75.-.A | 0.41506487 | 0.287158065 |
| 7740425 | 3000 | 51.C.A;75.-.G | 0.413952218 | 0.309260684 |
| 2271544 | 3001 | 0.T.-;122.A.C | 0.412907976 | 0.313660504 |
| 8154715 | 3002 | 76.G.-;78.A.C;132.G.T | 0.412514098 | 0.330364487 |
| 2684548 | 3003 | 0.T.-;2.A.C;132.GA.CC | 0.412508844 | 0.221325092 |
| 1042081 | 3004 | -17.C.A;77.-.A | 0.412076905 | 0.146558067 |
| 14808586 | 3005 | -29.A.C;82.AA.-- | 0.411847708 | 0.267953299 |
| 8106752 | 3006 | 76.GG.-A;113.A.C | 0.411607169 | 0.272676178 |
| 8447956 | 3007 | 80.A.-;127.T.G | 0.410631483 | 0.234388742 |
| 8128664 | 3008 | 75.-.C;131.A.G | 0.409653057 | 0.338241648 |
| 1291175 | 3009 | -15.T.G;2.A.-;75.-.G | 0.409209938 | 0.3796168 |
| 1253907 | 3010 | -15.T.G;73.A.- | 0.408538157 | 0.239463307 |
| 8128396 | 3011 | 128.T.C;75.-.C | 0.407284315 | 0.25239378 |
| 14084593 | 3012 | -20.A.C;75.-.G | 0.406446952 | 0.340365597 |
| 2661890 | 3013 | 0.T.-;2.A.C;76.G.-;129.C.A | 0.406369959 | 0.358795066 |
| 8598917 | 3014 | 70.-.T;82.A.- | 0.40571344 | 0.363210997 |
| 8519493 | 3015 | 130.--T.TAG;133.A.G;76.GG.-T | 0.404790669 | 0.16478942 |
| 2655861 | 3016 | 0.T.-;2.A.C;76.GG.-A;132.G.C | 0.404290669 | 0.211492433 |
| 8554353 | 3017 | 74.-C.TA | 0.403856841 | 0.278654898 |
| 6557545 | 3018 | 18.C.A;76.GG.-T | 0.403794566 | 0.248846831 |
| 1247115 | 3019 | -15.T.G;77.-.A | 0.402928751 | 0.162190367 |
| 15450484 | 3020 | -30.C.G;74.-.G | 0.401571837 | 0.368581694 |
| 8105724 | 3021 | 76.GG.-A;131.AG.CC | 0.400845215 | 0.31233423 |
| 14644689 | 3022 | -29.A.C;0.T.-;2.A.C;75.-.A | 0.400778989 | 0.380620086 |
| 8558610 | 3023 | 74.-.T;129.C.G | 0.400473999 | 0.215598514 |
| 8357449 | 3024 | 87.-.G;124.T.G | 0.4003889 | 0.279813501 |
| 15738093 | 3025 | -32.G.T;78.A.- | 0.39957936 | 0.178694312 |
| 8161146 | 3026 | 79.G.-;132.G.T | 0.39905064 | 0.197100501 |
| 827638 | 3027 | -21.C.A;76.GG.-C | 0.399045423 | 0.381135643 |
| 14647317 | 3028 | -29.A.C;0.T.-;2.A.C;74.AT.-G | 0.398936731 | 0.337066703 |
| 8431948 | 3029 | 82.AA.-T;132.G.T | 0.3962767 | 0.282558622 |
| 14344384 | 3030 | -25.A.C;75.-.A | 0.395805888 | 0.31302797 |
| 8508448 | 3031 | 78.A.T;132.G.C | 0.394920905 | 0.354687022 |
| 8150265 | 3032 | 77.-.A;132.G.C | 0.394788052 | 0.232297315 |
| 8654330 | 3033 | 65.GC.-T;78.A.- | 0.394710446 | 0.293953197 |
| 8093514 | 3034 | 75.-.A;123.A.C | 0.393696908 | 0.309225612 |
| 8352775 | 3035 | 86.C.-;130.T.G | 0.39207924 | 0.217323726 |
| 8066628 | 3036 | 74.T.-;130.T.G | 0.391719849 | 0.262493357 |
| 15168618 | 3037 | -29.A.G;76.G.-;78.A.T | 0.389830815 | 0.33561224 |
| 672344 | 3038 | -23.C.A;78.A.- | 0.389587037 | 0.321933192 |
| 8586257 | 3039 | 73.AT.-G;132.G.T | 0.388395464 | 0.296363207 |
| 8105301 | 3040 | 76.GG.-A;124.T.G | 0.388226799 | 0.287549837 |
| 8212901 | 3041 | 86.-.C;131.AG.CC | 0.386148792 | 0.352659282 |
| 13588657 | 3042 | -10.A.C;76.G.- | 0.384737506 | 0.348068257 |
| 728974 | 3043 | -22.T.A;75.-.G | 0.384109233 | 0.325342595 |
| 8448212 | 3044 | 80.A.-;132.G.T | 0.382825545 | 0.197802389 |
| 8128219 | 3045 | 75.-.C;125.T.G | 0.382212437 | 0.342348339 |
| 8084164 | 3046 | 130.--T.TAG;133.A.G;74.-.G | 0.380674413 | 0.324462071 |
| 13800992 | 3047 | -14.A.C;1.TA.-- | 0.380502059 | 0.379567092 |
| 8084111 | 3048 | 74.-.G;130.T.G | 0.379838914 | 0.284915658 |
| 14348272 | 3049 | -25.A.C;87.-.G | 0.375787656 | 0.227005333 |
| 8032112 | 3050 | 72.-.C;121.C.A | 0.374984841 | 0.316858242 |
| 8599500 | 3051 | 70.-.T;80.A.- | 0.374957082 | 0.306856796 |
| 14647476 | 3052 | -29.A.C;0.T.-;2.A.C;73.AT.-G | 0.374849427 | 0.287178991 |
| 8637349 | 3053 | 66.CT.-G;82.A.- | 0.374748495 | 0.369535198 |
| 14059318 | 3054 | 2.A.C;0.T.-;-20.A.C | 0.374318246 | 0.261266848 |
| 5590089 | 3055 | 10.T.C;87.-.T | 0.372525513 | 0.344891 |
| 8105685 | 3056 | 76.GG.-A;130.--T.TAG;133.A.G | 0.372066359 | 0.23292177 |
| 2687214 | 3057 | 0.T.-;2.A.C;113.A.G | 0.370636094 | 0.260077315 |
| 8605752 | 3058 | 73.A.-;82.A.- | 0.369387324 | 0.344859167 |
| 8066727 | 3059 | 74.T.-;131.AG.CC | 0.366894432 | 0.284573613 |
| 872410 | 3060 | -21.C.-;76.G.- | 0.366441507 | 0.282320025 |
| 13168637 | 3061 | -1.G.T;75.-.C | 0.36622796 | 0.325690795 |
| 442575 | 3062 | -27.C.A;77.-.A | 0.365239949 | 0.148841169 |
| 670080 | 3063 | -23.C.A;76.GG.-A | 0.365193115 | 0.229198474 |
| 2536818 | 3064 | 1.T.C;3.C.- | 0.365058878 | 0.278411465 |
| 15239473 | 3065 | -29.A.G;2.A.-;75.-.A | 0.364330715 | 0.307941812 |
| 8599361 | 3066 | 70.-.T;82.AA.-T | 0.364075981 | 0.203190312 |
| 8447558 | 3067 | 80.A.-121.C.A | 0.363793637 | 0.189981353 |
| 8032400 | 3068 | 72.-.C;132.G.C | 0.362895096 | 0.277357076 |
| 2591751 | 3069 | 0.T.-;2.A.C;33.C.A | 0.362710162 | 0.289879239 |
| 8151955 | 3070 | 76.G.-;82.A.G | 0.361619023 | 0.2931134 |
| 829720 | 3071 | -21.C.A;78.A.- | 0.361572174 | 0.340207762 |
| 8633205 | 3072 | 66.CT.-G.133.A.C | 0.361235295 | 0.177612583 |
| 8367621 | 3073 | 86.-.G;131.A.C | 0.360882293 | 0.14994125 |
| 8652746 | 3074 | 65.GC.-T | 0.359676845 | 0.34117811 |
| 8641968 | 3075 | 66.CT.-- | 0.359510719 | 0.335128609 |
| 8489994 | 3076 | 76.-.G;125.T.G | 0.359266847 | 0.243082633 |
| 2271196 | 3077 | 0.T.-;134.G.T | 0.357221231 | 0.333356566 |
| 2684526 | 3078 | 0.T.-;2.A.C;132.G.A | 0.357103171 | 0.210774129 |
| 6557839 | 3079 | 18.C.A;74.-.T | 0.356398057 | 0.194388522 |
| 15057882 | 3080 | -29.A.G;0.T.-;2.A.C;74.T.- | 0.355573213 | 0.347677573 |
| 14812029 | 3081 | -29.A.C;78.A.G | 0.354936599 | 0.331966329 |
| 8565161 | 3082 | 75.CG.-T;127.T.G | 0.354149416 | 0.290483884 |
| 1042365 | 3083 | -17.C.A;77.GA.-- | 0.352230794 | 0.264271374 |
| 1114842 | 3084 | -16.C.A;75.-.C | 0.351420163 | 0.323308043 |
| 3011677 | 3085 | 1.TA.--;128.T.G | 0.349353976 | 0.272131853 |
| 8367521 | 3086 | 86.-.G;129.C.A | 0.349102113 | 0.128912924 |
| 8545111 | 3087 | 75.-.G;82.A.G | 0.348846687 | 0.279265182 |
| 13670603 | 3088 | -12.G.T;0.T.-;2.A.C | 0.346705159 | 0.220809539 |
| 8152309 | 3089 | 76.G.-;80.A.G | 0.344879701 | 0.240148808 |
| 14635704 | 3090 | -29.A.C;0.T.-;78.A.- | 0.343977628 | 0.269327054 |
| 8101708 | 3091 | 75.CGG.-AT | 0.343807137 | 0.263179626 |
| 15738145 | 3092 | -32.G.T;76.-.G | 0.343373872 | 0.282940777 |
| 14351983 | 3093 | -25.A.C;73.A.- | 0.342166961 | 0.317506007 |
| 8066472 | 3094 | 74.T.-;127.T.G | 0.341452423 | 0.218881305 |
| 8134358 | 3095 | 75.-G.CT | 0.340668573 | 0.260397851 |
| 8603055 | 3096 | 73.A.-;129.C.A | 0.339516932 | 0.284512591 |
| 1251152 | 3097 | -15.T.G;82.AA.-T | 0.337292843 | 0.221583879 |
| 1005071 | 3098 | -17.C.A;1.TA.-- | 0.335312695 | 0.306486266 |
| 8137618 | 3099 | 76.G.-;104.C.A | 0.335162523 | 0.190958854 |
| 15158102 | 3100 | -29.A.G;72.-.C | 0.334668341 | 0.245386507 |
| 8129152 | 3101 | 75.-.C;121.C.T | 0.334449323 | 0.186487396 |
| 8208002 | 3102 | 88.G.-;130.T.G | 0.333618091 | 0.136446113 |
| 3581291 | 3103 | 2.-.A;72.-.C | 0.331079889 | 0.299960469 |
| 1251375 | 3104 | -15.T.G;80.A.- | 0.330673201 | 0.237553781 |
| 8128320 | 3105 | 75.-.C;127.T.C | 0.329450929 | 0.31539949 |
| 8356949 | 3106 | 87.-.G;118.T.G | 0.328766524 | 0.276642735 |
| 8552259 | 3107 | 75.C.-;86.C.- | 0.328683252 | 0.274572035 |
| 830221 | 3108 | -21.C.A;74.-.T | 0.328073756 | 0.279164881 |
| 2820364 | 3109 | 0.T.-;2.A.C;18.C.T | 0.328071337 | 0.303059134 |
| 15456319 | 3110 | -30.C.G;76.-.T | 0.327788273 | 0.239917243 |
| 8470089 | 3111 | 78.-.C;126.C.A | 0.327502065 | 0.285083789 |
| 8161135 | 3112 | 79.G.-;133.A.C | 0.327120166 | 0.249238373 |
| 8481813 | 3113 | 78.A.-;119.C.T | 0.326577601 | 0.263148897 |
| 2684845 | 3114 | 0.T.-;2.A.C;126.C.T | 0.326497023 | 0.268527975 |
| 8128793 | 3115 | 75.-.C;126.C.T | 0.325657328 | 0.244960408 |
| 15405296 | 3116 | -30.C.G;0.T.- | 0.324922115 | 0.303112615 |
| 8595845 | 3117 | 70.-.T;129.C.A | 0.323993445 | 0.292377507 |
| 8105737 | 3118 | 76.GG.-A;131.A.C;133.A.C | 0.323238212 | 0.214800697 |
| 8470189 | 3119 | 78.-.C;129.C.A | 0.323151711 | 0.297959942 |
| 14245594 | 3120 | -24.G.T;80.A.- | 0.323015835 | 0.259376759 |
| 1251224 | 3121 | -15.T.G;81.GA.-T | 0.322672044 | 0.236717429 |
| 7939926 | 3122 | 65.G.-;76.G.- | 0.321874555 | 0.229114823 |
| 8648998 | 3123 | 65.G.T;76.G.- | 0.32161445 | 0.165407591 |
| 14098317 | 3124 | -20.A.C;2.A.- | 0.321338341 | 0.261130203 |
| 8032447 | 3125 | 72.-.C;131.A.C | 0.320310642 | 0.25131762 |
| 8061102 | 3126 | 74.T.-;76.G.C | 0.320134619 | 0.17974794 |
| 8481588 | 3127 | 78.A.-;120.C.T | 0.31991061 | 0.266621576 |
| 8565286 | 3128 | 75.CG.-T;130.T.C | 0.319658388 | 0.299836722 |
| 14245896 | 3129 | -24.G.T;76.-.G | 0.318978655 | 0.198135025 |
| 8066445 | 3130 | 74.T.-;127.T.C | 0.318741324 | 0.229575007 |
| 8150200 | 3131 | 77.-.A;129.C.A | 0.318392177 | 0.222652224 |
| 8479230 | 3132 | 78.A.-;118.T.G | 0.315585221 | 0.212655987 |
| 8482576 | 3133 | 78.A.-;113.A.C | 0.313923006 | 0.235801574 |
| 2271423 | 3134 | 0.T.-;123.A.C | 0.313151728 | 0.262740752 |
| 13907909 | 3135 | -14.A.G;0.T.-;2.A.C | 0.312602248 | 0.24235172 |
| 8066743 | 3136 | 74.T.-;131.A.C;133.A.C | 0.311512836 | 0.213517827 |
| 8352697 | 3137 | 86.C.-;128.T.G | 0.31093017 | 0.185786592 |
| 301021 | 3138 | -28.G.C;0.T.-;2.A.C | 0.308009842 | 0.177963593 |
| 8480313 | 3139 | 78.A.-;125.T.G | 0.307352894 | 0.265386782 |
| 8136771 | 3140 | 76.G.-;87.C.A | 0.305748033 | 0.204149437 |
| 8019966 | 3141 | 72.-.A;82.A.- | 0.305426544 | 0.276125022 |
| 8632613 | 3142 | 66.CT.-G;121.C.A | 0.305245351 | 0.18051425 |
| 8583599 | 3143 | 73.AT.-G;88.G.- | 0.305036767 | 0.281668863 |
| 8475891 | 3144 | 78.A.-;88.G.- | 0.304225711 | 0.24315761 |
| 8567785 | 3145 | 75.C.T;77.-.A | 0.303944466 | 0.161149893 |
| 8448066 | 3146 | 80.A.-;129.C.A | 0.303325704 | 0.215444753 |
| 8136691 | 3147 | 76.G.-;86.C.A | 0.302433752 | 0.195854751 |
| 15059855 | 3148 | -29.A.G;0.T.-;2.A.C;66.CT.-G | 0.301250125 | 0.258032296 |
| 13171297 | 3149 | -1.G.T;76.-.G | 0.300469679 | 0.249568302 |
| 8470230 | 3150 | 78.-.C;130.T.G | 0.299543757 | 0.27947901 |
| 8142877 | 3151 | 76.G.-;134.G.C | 0.29949224 | 0.197954128 |
| 555214 | 3152 | -26.T.C;76.G.- | 0.29846809 | 0.182034813 |
| 446048 | 3153 | -27.C.A;80.A.- | 0.298324534 | 0.210212488 |

TABLE 26

| index | SEQ ID NO | muts_1indexed | MI | 95% CI |
|---|---|---|---|---|
| 8436528 | 3154 | 81.GA.-T;121.C.A | 0.297090048 | 0.283427352 |
| 8353141 | 3155 | 86.C.-;122.A.C | 0.296049987 | 0.245918877 |
| 8565426 | 3156 | 75.CG.-T;131.A.G | 0.295840924 | 0.235610502 |
| 8132576 | 3157 | 75.-.C;89.-.C | 0.295816698 | 0.21575762 |
| 8092121 | 3158 | 75.-.A;116.T.G | 0.295438612 | 0.276704748 |
| 8633166 | 3159 | 66.CT.-G;132.G.C | 0.295238555 | 0.137541162 |
| 8142165 | 3160 | 76.G.-;124.T.C | 0.294668253 | 0.252511967 |
| 2686290 | 3161 | 0.T.-;2.A.C;114.G.T | 0.294611939 | 0.235882425 |
| 8161038 | 3162 | 79.G.-;129.C.A | 0.293458957 | 0.265995213 |
| 13853578 | 3163 | -14.A.C;76.-.T | 0.292814241 | 0.239208093 |
| 807836 | 3164 | -21.C.A;1.TA.-- | 0.291985874 | 0.265062731 |
| 8469754 | 3165 | 78.-.C;119.C.A | 0.290688734 | 0.158231713 |
| 8137474 | 3166 | 76.G.-;101.C.A | 0.290545033 | 0.225586567 |
| 8160587 | 3167 | 79.G.-;120.C.A | 0.290485378 | 0.16140082 |
| 8142955 | 3168 | 76.G.-;131.AGA.CCC | 0.289861064 | 0.156100467 |
| 8762708 | 3169 | 56.G.T;75.-.G | 0.288589286 | 0.245071065 |
| 14635887 | 3170 | 0.T.-;-29.A.C;75.-.G | 0.287655949 | 0.220550516 |
| 15455571 | 3171 | -30.C.G;78.-.C | 0.286554251 | 0.151262545 |
| 8066265 | 3172 | 74.T.-;124.T.G | 0.284557684 | 0.18450021 |
| 8436842 | 3173 | 81.GA.-T;130.T.G | 0.283443437 | 0.227668014 |
| 13846354 | 3174 | -14.A.C;79.G.- | 0.282193081 | 0.194513828 |
| 8490993 | 3175 | 76.-.G;121.C.T | 0.281487779 | 0.237968585 |
| 14646258 | 3176 | -29.A.C;0.T.-;2.A.C;87.-.T | 0.281390861 | 0.280842128 |
| 8431378 | 3177 | 82.AA.-T;120.C.A | 0.279359971 | 0.217352128 |
| 8431703 | 3178 | 82.AA.-T;126.C.A | 0.278958399 | 0.248775754 |
| 447910 | 3179 | -27.C.A;73.AT.-G | 0.27887466 | 0.214623934 |
| 8066683 | 3180 | 74.T.-;130.--T.TAG;133.A.G | 0.278590377 | 0.236479801 |
| 2760011 | 3181 | 0.T.-;2.A.C;58.G.T | 0.27816451 | 0.250084418 |
| 3012063 | 3182 | 1.TA.--;123.A.C | 0.277695499 | 0.270902767 |
| 13855018 | 3183 | -14.A.C;73.A.- | 0.277345113 | 0.240410092 |
| 8447252 | 3184 | 80.A.-;119.C.A | 0.276750412 | 0.261342977 |
| 8489127 | 3185 | 76.-.G;118.T.G | 0.275614164 | 0.268649953 |
| 8526408 | 3186 | 76.-.T;126.C.A | 0.275422119 | 0.186856595 |
| 8446211 | 3187 | 80.A.-;115.T.G | 0.273001999 | 0.176712389 |
| 8431937 | 3188 | 82.AA.-T;133.A.C | 0.272461593 | 0.215640473 |
| 6558231 | 3189 | 18.C.A;73.A.- | 0.270722227 | 0.209417884 |
| 8159873 | 3190 | 79.G.-;115.T.G | 0.270544898 | 0.219973209 |
| 8602463 | 3191 | 73.A.-;119.C.A | 0.267631124 | 0.229610693 |
| 2684642 | 3192 | 0.T.-;2.A.C;131.AGA.CCC | 0.267606676 | 0.193922958 |
| 8143095 | 3193 | 76.G.-;126.C.G | 0.26607975 | 0.205850153 |
| 1042210 | 3194 | -17.C.A;79.G.- | 0.263898352 | 0.153341127 |
| 15452123 | 3195 | -30.C.G;88.G- | 0.262802964 | 0.246339122 |
| 13852053 | 3196 | -14.A.C;80.A.- | 0.262449421 | 0.238482785 |
| 8435985 | 3197 | 81.GA.-T;115.T.G | 0.261537752 | 0.210117266 |
| 223220 | 3198 | -30.C.A;76.G.- | 0.260927881 | 0.212705604 |
| 12148242 | 3199 | 2.A.-;124.T.C | 0.259970416 | 0.231655778 |
| 8602984 | 3200 | 73.A.-;127.T.G | 0.259333216 | 0.17429791 |
| 318643 | 3201 | -28.G.C;75.-.C | 0.258711926 | 0.253858239 |
| 15451555 | 3202 | -30.C.G;79.G.- | 0.258610617 | 0.228040833 |
| 8436802 | 3203 | 81.GA.-T;129.C.A | 0.258102815 | 0.221392597 |
| 8512529 | 3204 | 76.G.-;78.A.T;131.A.C | 0.256573774 | 0.192299447 |
| 8519060 | 3205 | 76.GG.-T;124.T.G | 0.254764495 | 0.17776839 |
| 1045581 | 3206 | -17.C.A;78.-.C | 0.254111585 | 0.16098974 |
| 13844608 | 3207 | -14.A.C;74.T.- | 0.251536336 | 0.230596398 |
| 13171509 | 3208 | -1.G.T;76.GG.-T | 0.251215355 | 0.178972378 |
| 8336250 | 3209 | 89.-.C;121.C.A | 0.247903737 | 0.177200161 |
| 15455277 | 3210 | -30.C.G;80.A.- | 0.24643105 | 0.215568133 |
| 8353027 | 3211 | 86.C.-;123.A.C | 0.245734783 | 0.146234159 |
| 8161013 | 3212 | 79.G.-;128.T.G | 0.245117825 | 0.184156133 |
| 8105760 | 3213 | 76.GG.-A;129.C.G | 0.243519956 | 0.200992141 |
| 8558713 | 3214 | 74.-.T;123.A.C | 0.243362245 | 0.217508129 |
| 2681904 | 3215 | 0.T.-;2.A.C;116.T.C | 0.243150168 | 0.227835889 |
| 8558310 | 3216 | 74.-.T;127.T.C | 0.238872167 | 0.164543464 |
| 2684449 | 3217 | 0.T-;2.A.C;130.T.C;132.G.C | 0.234640315 | 0.191407277 |
| 15052207 | 3218 | -29.A.G;0.T.-;75.-.G | 0.232527238 | 0.228978007 |
| 8524468 | 3219 | 76.G.T;78.A.- | 0.231822737 | 0.184427214 |
| 7490514 | 3220 | 36.C.A;76.GG.-A | 0.230612085 | 0.201072386 |
| 8633217 | 3221 | 66.CT.-G;132.G.T | 0.225041391 | 0.188349309 |
| 8069615 | 3222 | 74.T.-;89.-.C | 0.224219112 | 0.182205253 |
| 15451403 | 3223 | -30.C.G;77.-.A | 0.22377016 | 0.141786542 |
| 8520167 | 3224 | 76.GG.-T;119.C.T | 0.222213862 | 0.181552856 |
| 10994911 | 3225 | 8.G.T;76.G.- | 0.221857972 | 0.186488557 |
| 2272784 | 3226 | 0.T.-;113.A.G | 0.217602613 | 0.188068889 |
| 8100983 | 3227 | 75.C.A;87.-.G | 0.20946824 | 0.207400395 |
| 13851721 | 3228 | -14.A.C;82.AA.-T | 0.208699774 | 0.190610953 |
| 8084086 | 3229 | 74.-.G;130.T.C | 0.207083817 | 0.200301272 |
| 8564034 | 3230 | 75.CG.-T;116.T.G | 0.206201826 | 0.195294871 |
| 1117838 | 3231 | -16.C.A;75.CG.-T | 0.205361121 | 0.20010844 |
| 14023671 | 3232 | -19.G.T;76.GG.-T | 0.205124123 | 0.18913669 |
| 8519544 | 3233 | 76.GG.-T;131.A.C;133.A.C | 0.201318374 | 0.159186928 |
| 8633185 | 3234 | 66.CT.-G | 0.199632516 | 0.137407357 |
| 14817545 | 3235 | -29.A.C;66.CT.-G | 0.199449017 | 0.147317397 |
| 1482006 | 3236 | -9.T.C;76.G.- | 0.199005805 | 0.183058025 |
| 14524849 | 3237 | -28.G.T;75.-.C | 0.198371675 | 0.181096792 |
| 8470132 | 3238 | 78.-.C;127.T.G | 0.197187102 | 0.191993677 |
| 7738954 | 3239 | 51.C.A;76.G.- | 0.188853628 | 0.174711687 |
| 1247296 | 3240 | -15.T.G;79.G.- | 0.188770966 | 0.162582829 |
| 8519864 | 3241 | 76.GG.-T;122.A.G | 0.187827314 | 0.124500437 |
| 1117512 | 3242 | -16.C.A;76.GG.-T | 0.185440387 | 0.166113954 |
| 15171788 | 3243 | -29.A.G;66.CT.-G | 0.184297092 | 0.119128778 |
| 8601732 | 3244 | 73.A.-;115.T.G | 0.182910648 | 0.17442519 |
| 6556220 | 3245 | 18.C.A;86.C.- | 0.182226427 | 0.124165253 |
| 8633071 | 3246 | 66.CT.-G;129.C.A | 0.174547902 | 0.164343167 |
| 8499488 | 3247 | 78.A.-;80.A.G | 0.170717115 | 0.165935562 |
| 8519321 | 3248 | 76.GG.-T;128.T.C | 0.169470546 | 0.133277047 |
| 14348190 | 3249 | -25.A.C;86.C.- | 0.164802634 | 0.107431366 |
| 321013 | 3250 | -28.G.C;74.-.T | 0.163668333 | 0.162660862 |

Approximately 140 modified gRNAs were generated, some by DME and some by targeted engineering, and assayed for their ability to disrupt expression of a target GFP reporter construct by creation of indels. Sequences for these gRNA variants are shown in Table 3. These modified gRNAs exclude modifications to the spacer region, and instead comprise different modified scaffolds (the portion of the sgRNA that interacts with the CRISPR protein, protein binding segment). gRNA scaffolds generated by DME include one or more deletions, substitutions, and insertions, which can consist of a single or several bases. The remaining gRNA variants were rationally engineered based on knowledge of thermostable RNA structures, and are either terminal fusions of ribozymes or insertions of highly stable stem loop sequences. Additional gRNAs were generated by combining gRNA variants. The results for select gRNA variants are shown in Table 27 below.

TABLE 27
Ability of select gRNA variants to disrupt GFP expression.

| | | Normalized | |
|---|---|---|---|
| | | Editing | |
| SEQ ID | | Activity (ave, | |
| NO: | NAME (Description) | 2 spacers n = 6) | Std. dev. |
| 5 | X2 reference | — | — |
| 2101 | phage replication stable | 1.42 | 0.22 |
| 2102 | Kissing loop_b1 | 1.17 | 0.11 |
| 2103 | Kissing loop_a | 1.18 | 0.03 |
| 2104 | 32, uysX hairpin | 1.89 | 0.11 |
| 2105 | PP7 | 1.08 | 0.04 |
| 2106 | 64, trip mut, extended stem truncation | 1.69 | 0.18 |
| 2107 | hyperstable tetraloop | 1.36 | 0.11 |
| 2108 | C18G | 1.22 | 0.42 |
| 2109 | T17G | 1.27 | 0.04 |
| 2110 | CUUCGG loop | 1.24 | 0.22 |
| 2111 | MS2 | 1.12 | 0.25 |
| 2112 | −1, A2G, −78, G77T | 1.00 | 0.18 |
| 2113 | QB | 1.44 | 0.25 |
| 2114 | 45, 44 hairpin | 0.24 | 0.41 |
| 2115 | U1A | 1.02 | 0.05 |
| 2116 | A14C, T17G | 0.86 | 0.01 |
| 2117 | CUUCGG loop modified | 0.75 | 0.04 |
| 2118 | Kissing loop_b2 | 0.99 | 0.06 |
| 2119 | −76:78, −83:87 | 0.97 | 0.01 |
| 2120 | −4 | 0.93 | 0.03 |
| 2121 | extended stem truncation | 0.73 | 0.02 |
| 2124 | −98:100 | 0.66 | 0.05 |
| 2125 | −1:5 | 0.45 | 0.05 |
| 2126 | −2163 | 0.57 | 0.02 |
| 2127 | =+G28, A82T, −84, | 0.56 | 0.04 |
| 2128 | =+51T | 0.52 | 0.03 |
| 2129 | −1:4, +G5A, +G86, | 0.09 | 0.21 |
| 2130 | 2174 | 0.34 | 0.09 |
| 2131 | +g72 | 0.34 | 0.24 |
| 2132 | shorten front, CUUCGG loop | 0.65 | 0.02 |
| | modified. extend extended | | |
| 2133 | A14C | 0.37 | 0.03 |
| 2134 | −1:3, +G3 | 0.45 | 0.16 |
| 2135 | =+C45, +T46 | 0.42 | 0.04 |
| 2136 | CUUCGG loop modified, fun start | 0.38 | 0.03 |
| 2137 | −74:75 | 0.18 | 0.04 |
| 2138 | \hat{ }T45 | 0.21 | 0.05 |
| 2139 | −69, −94 | 0.24 | 0.09 |
| 2140 | −94 | 0.01 | 0.01 |
| 2141 | modified CUUCGG, minus T in 1st triplex | 0.04 | 0.03 |
| 2142 | −1:4, +C4, A14C, T17G, +G72, −76:78, −83:87 | 0.16 | 0.03 |
| 2143 | T1C, −73 | 0.06 | 0.06 |
| 2144 | Scaffold uuCG, stem uuCG. Stem swap, t shorten | 0.01 | 0.09 |
| 2145 | Scaffold uuCG, stem uuCG. Stem swap | 0.04 | 0.03 |
| 2146 | 0.0090408 | 0.06 | 0.04 |
| 2147 | no stem Scaffold uuCG | −0.11 | 0.02 |
| 2148 | no stem Scaffold uuCG, fun start | −0.06 | 0.02 |
| 2149 | Scaffold uuCG, stem uuCG, fun start | −0.02 | 0.02 |
| 2150 | Pseudoknots | −0.01 | 0.01 |
| 2151 | Scaffold uuCG, stem uuCG | −0.05 | 0.01 |
| 2152 | Scaffold uuCG, stem uuCG, no start | −0.04 | 0.02 |
| 2153 | Scaffold uuCG | −0.12 | 0.07 |
| 2154 | +GCTC36 | −0.20 | 0.05 |
| 2155 | G quadriplex telomere basket + ends | −0.21 | 0.02 |
| 2156 | G quadriplex M3q | −0.25 | 0.04 |
| 2157 | G quadriplex telomere basket no ends | −0.17 | 0.04 |
| 2159 | Sarcin-ricin loop | 0.40 | 0.03 |
| 2160 | uvsX, C18G | 1.94 | 0.06 |
| 2161 | truncated stem loop, C18G, trip mut (T10C) | 1.97 | 0.16 |
| 2162 | short phage rep, C18G | 1.91 | 0.17 |
| 2163 | phage rep loop, C18G | 1.72 | 0.13 |
| 2164 | +G18, stacked onto 64 | 1.44 | 0.08 |
| 2165 | truncated stem loop, C18G, −1 A2G | 1.63 | 0.40 |
| 2166 | phage rep loop, C18G, trip mut (T10C) | 1.76 | 0.12 |
| 2167 | short phage rep, C18G, trip mut (T10C) | 1.20 | 0.09 |
| 2168 | uvsX, trip mut (T10C) | 1.54 | 0.12 |
| 2169 | truncated stem loop | 1.50 | 0.10 |
| 2170 | +A17, stacked onto 64 | 1.54 | 0.13 |
| 2171 | 3′ HDV genomic ribozyme | 1.13 | 0.13 |
| 2172 | phage rep loop, trip mut (T10C) | 1.39 | 0.10 |
| 2173 | −79:80 | 1.33 | 0.05 |
| 2174 | short phage rep, trip mut (T10C) | 1.19 | 0.10 |
| 2175 | extra truncated stem loop | 1.08 | 0.05 |
| 2176 | T17G, C18G | 0.94 | 0.09 |
| 2177 | short phage rep | 1.11 | 0.05 |
| 2178 | uvsX, C18G, −1 A2G | 0.62 | 0.08 |
| 2179 | uvsX, C18G, trip mut (T10C), −1 A2G, | 1.06 | 0.08 |
| | HDV −99 G65U | | |
| 2180 | 3′ HDV antigenomic ribozyme | 1.20 | 0.07 |
| 2181 | uvsX, C18G, trip mut (T10C), −1 A2G, | 0.95 | 0.03 |
| | HDV AA(98:99)C | | |
| 2182 | 3′ HDV ribozyme (Lior Nissim, Timothy Lu) | 1.08 | 0.01 |
| 2183 | TAC(1:3)GA, stacked onto 64 | 0.92 | 0.04 |
| 2184 | uvsX, −1 A2G | 1.46 | 0.13 |
| 2185 | truncated stem loop, C18G, trip mut (T10C), | 0.80 | 0.02 |
| | −1 A2G, HDV −99 G65U | | |
| 2186 | short phage rep, C18G, trip mut (T10C), | 0.80 | 0.05 |
| | −1 A2G, HDV −99 G65U | | |
| 2187 | 3′ sTRSV WT viral Hammerhead ribozyme | 0.98 | 0.03 |
| 2188 | short phage rep, C18G, −1 A2G | 1.78 | 0.18 |
| 2189 | short phage rep, C18G, trip mut (T10C), | 0.81 | 0.08 |
| | −1 A2G, 3′ genomic HDV | | |
| 2190 | phage rep loop, C18G, trip mut (T10C), | 0.86 | 0.07 |
| | −1 A2G, HDV −99 G65U | | |
| 2191 | 3′ HDV ribozyme (Owen Ryan, Jamie Cate) | 0.78 | 0.04 |
| 2192 | phage rep loop, C18G, −1 A2G | 0.70 | 0.08 |
| 2193 | \hat{ }C55 | 0.78 | 0.03 |
| 2194 | −78, G77T | 0.73 | 0.07 |
| 2195 | \hat{ }G1 | 0.73 | 0.10 |
| 2196 | short phage rep, −1 A2G | 0.66 | 0.11 |
| 2197 | truncated stem loop, C18G, trip mut (T10C), | 0.68 | 0.09 |
| | −1 A2G | | |
| 2198 | −1, A2G | 0.54 | 0.07 |
| 2199 | truncated stem loop, trip mut (T10C), −1 A2G | 0.40 | 0.03 |
| 2200 | uvsX, C18G, trip mut (T10C), −1 A2G | 0.35 | 0.11 |
| 2201 | phage rep loop, −1 A2G | 0.96 | 0.05 |
| 2202 | phage rep loop, trip mut (T10C), −1 A2G | 0.49 | 0.06 |
| 2203 | phage rep loop, C18G, trip mut (T10C), −1 A2G | 0.73 | 0.13 |
| 2204 | truncated stem loop, C18G | 0.59 | 0.02 |
| 2205 | uvsX, trip mut (T10C), −1 A2G | 0.56 | 0.08 |
| 2206 | truncated stem loop, −1 A2G | 0.89 | 0.07 |
| 2207 | short phage rep, trip mut (T10C), −1 A2G | 0.37 | 0.12 |
| 2208 | 5′HDV ribozyme (Owen Ryan, Jamie Cate) | 0.39 | 0.03 |
| 2209 | 5′HDV genomic ribozyme | 0.35 | 0.06 |
| 2210 | truncated stem loop, C18G, trip mut (T10C), | 0.24 | 0.04 |
| | −1 A2G, HDV AA(98:99)C | | |
| 2211 | 5′env25 pistol ribozyme (with an added | 0.33 | 0.07 |
| | CUUCGG loop) | | |
| 2212 | 5′HDV antigenomic ribozyme | 0.17 | 0.01 |
| 2213 | 3′ Hammerhead ribozyme (Lior Nissim, | 0.09 | 0.02 |
| | Timothy Lu) guide scaffold scar | | |
| 2214 | +A27, stacked onto 64 | 0.03 | 0.03 |
| 2215 | 5′Hammerhead ribozyme (Lior Nissim, | 0.18 | 0.03 |
| | Timothy Lu) smaller scar | | |
| 2216 | phage rep loop, C18G, trip mut (T10), | 0.13 | 0.04 |
| | −1 A2G, HDV AA(98:99)C | | |
| 2217 | −27, stacked onto 64 | 0.00 | 0.03 |
| 2218 | 3′ Hatchet | 0.09 | 0.01 |
| 2219 | 3′ Hammerhead ribozyme (Lior Nissim, | 0.05 | 0.03 |
| | Timothy Lu) | | |
| 2220 | 5′Hatchet | 0.04 | 0.03 |
| 2221 | 5′HDV ribozyme (Lior Nissim, Timothy Lu) | 0.08 | 0.01 |
| 2222 | 5′Hammerhead ribozyme (Lior Nissim, | 0.22 | 0.01 |
| | Timothy Lu) | | |
| 2223 | 3′ HH15 Minimal Hammerhead ribozyme | 0.01 | 0.01 |
| 2224 | 5′ RBMX recruiting motif | −0.08 | 0.03 |
| 2225 | 3′ Hammerhead ribozyme (Lior Nissim, | −0.04 | 0.02 |
| | Timothy Lu) smaller scar | | |
| 2226 | 3′ env25 pistol ribozyme (with an added | −0.01 | 0.01 |
| | CUUCGG loop) | | |
| 2227 | 3′ Env-9 Twister | −0.17 | 0.02 |
| 2228 | +ATTATCTCATTACT25 | −0.18 | 0.27 |
| 2229 | 5′Env-9 Twister | −0.02 | 0.01 |
| 2230 | 3′ Twisted Sister 1 | −0.27 | 0.02 |
| 2231 | no stem | −0.15 | 0.03 |
| 2232 | 5′HH15 Minimal Hammerhead ribozyme | −0.18 | 0.04 |
| 2233 | 5′Hammerhead ribozyme (Lior Nissim, | −0.14 | 0.01 |
| | Timothy Lu) guide scaffold scar | | |
| 2234 | 5′Twisted Sister 1 | −0.14 | 0.04 |
| 2235 | 5′sTRSV WT viral Hammerhead ribozyme | −0.15 | 0.02 |
| 2236 | 148, =+G55, stacked onto 64 | 3.40 | 0.18 |
| 2239 | 175, trip mut, extended stem truncation, | 1.18 | 0.09 |
| | with [T] deletion at 5′ end | | |

Although guide stability can be measured thermodynamically (for example, by analyzing melting temperatures) or kinetically (for example, using optical tweezers to measure folding strength), without wishing to be bound by any theory it is believed that a more stable sgRNA bolsters CRISPR editing efficiency. Thus, editing efficiency was used as the primary assay for improved guide function.

The activity of the gRNA scaffold variants was assayed using E6 and E7 spacers targeting GFP. The starting sgRNA scaffold in this case was a reference Planctomyces CasX tracr RNA fused to a Planctomyces Crispr RNA (crRNA) using a “GAAA” stem loop (SEQ ID NO: 5). The activity of variant gRNAs shown in Table 27 was normalized to the activity of this starting, or base, sgRNA scaffold.

The sgRNA scaffold was cloned into a small (less than 3 kilobase pair) plasmid with a 3′ type II restriction enzyme site for dropping in different spacers. The spacer region of the sgRNA is the part of the sgRNA interacts with the target DNA, and does not interact directly with the CasX protein. Thus, scaffold changes should be spacer independent. One way to achieve this is by executing sgRNA DME and testing sgRNA variants using several distinct spacers, such as the E6 and E7 spacers targeting GFP. This reduces the possibility of creating an sgRNA scaffold variant that works well with one spacer sequence targeting one genetic target, but not other spacer sequences directed to other targets. For the data shown in Table 27, the E6 and E7 spacer sequences targeting GFP were used. Repression of GFP expression by sgRNA variants was normalized to GFP repression by the sgRNA starting scaffold of SEQ ID NO: 5 assayed with the same spacer sequence(s).

Activity of select sgRNA variants is shown in FIGS. 5A and 5B, mean change in activity is shown in Table 27, and sgRNA variant sequences are provided in Table 3. sgRNA variants with increased activity were tested in HEK293 cells as described in Example 1.

### Example 4: Mutagenesis of CasX Protein Produces Improved Variants

A selectable, mammalian-expression plasmid was constructed that included a reference, also referred to herein as starting or base, CasX protein sequence, an sgRNA scaffold, and a destination sequence that can be replaced by spacer sequences. In this case, the starting CasX protein was SEQ ID NO: 2, the wild type Planctomycetes CasX sequence and the scaffold was the wild type sgRNA scaffold of SEQ ID NO: 5. This destination plasmid was digested using the appropriate restriction enzyme following manufacturer's protocol. Following digestion, the digested DNA was purified using column purification according to manufacturer's protocol. The E6 and E7 spacer oligos targeting GFP were annealed in 10 uL of annealing buffer. The annealed oligos were ligated to the purified digested backbone using a Golden Gate ligation reaction. The Golden Gate ligation product was transformed into chemically competent bacterial cells and plated onto LB agar plates with the appropriate antibiotic. Individual colonies were picked, and the GFP spacer insertion was verified via Sanger sequencing.

The following methods were used to construct a DME library of CasX variant proteins. The functional Plm CasX system, which is a 978 residue multi-domain protein (SEQ ID NO: 2) can function in a complex with a 108 bp sgRNA scaffold (SEQ ID NO: 5), with an additional 3′ 20 bp variable spacer sequence, which confers DNA binding specificity. Construction of the comprehensive mutation library thus required two methods: one for the protein, and one for the sgRNA. Plasmid recombineering was used to construct a DME protein library of CasX variant proteins. PCR-based mutagenesis was used to construct an RNA library of the sgRNA. Importantly, the DME approach can make use of a variety of molecular biology techniques. The techniques used for genetic library construction can be variable, while the design and scope of mutations encompasses the DME method.

In designing DME mutations for the reference CasX protein, synthetic oligonucleotides were constructed as follows: for each codon, three types of oligonucleotides were synthesized. First, the substitution oligonucleotide replaced the three nucleotides of the codon with one of 19 possible alternative codons which code for the 19 possible amino acid mutations. 30 base pair flanking regions of perfect homology to the target gene allow programmable targeting of these mutations. Second, a similar set of 20 synthetic oligonucleotides encoded the insertion of single amino acids. Here, rather than replace the codon, a new region consisting of three base pairs was inserted between the codon and the flanking homology region. Twenty different sets of three nucleotides were inserted, corresponding to new codons for each of the twenty amino acids. Larger insertions can be built identically but will contain an additional three, six, or nine base pairs, encoding all possible combinations of two, three, or four amino acids. Third, an oligonucleotide was designed to remove the three base pairs comprising the codon, thus deleting the amino acid. As above, oligonucleotides can be designed to delete one, two, three, or four amino acids. Plasmid recombineering was then used to recombine these synthetic mutations into a target gene of interest, however other molecular biology methods can be used in its place to accomplish the same goal.

Table 28 shows fold enrichment of CasX variant protein DME libraries created from the reference protein of SEQ ID NO: 2, which were then subjected to DME selection/screening processes.

In Table 28 below, the read counts associated with each of the listed variants was determined. Each variant was defined by its position (0-indexed), reference base, and alternate base. Only sequences with at least 10 reads (summed) across samples were analyzed, to filter from 457K variants to 60K variants. An insertion at position i indicates an inserted base between position i-1 and i (i.e., before the indicated position). ‘counts’ indicates the sequencing-depth normalized read count per sequence per sample. Technical replicates were combined by taking the geometric mean. ‘log2enrichment’ gives the median enrichment (using a pseudocount of 10) across each context, or across all samples, after merging for technical replicates. Each context was normalized by its own naive sample. Finally, the ‘log2enrichment_err’ gives the ‘confidence interval’ on the mean log2 enrichment. It is the std. deviation of the enrichment across samples *2/sqrt of the number of samples. Below, only the sequences with median log2enrichment−log2enrichment_err>0 are shown (60274 sequences examined).

The computational protocol used to generate Table 28 was as follows: each sample library was sequenced on an Illumina HiSeq for 150 cycles paired end (300 cycles total). Reads were trimmed to remove adapter sequences, and aligned to a reference sequence. Reads were filtered if they did not align to the reference, or if the expected number of errors per read was high, given the phred base quality scores. Reads that aligned to the reference sequence, but did not match exactly, were assessed for the protein mutation that gave rise to the mismatch, by aligning the encoded protein sequence of the read to the protein sequence of the reference at the aligned location. Any consecutive variants were grouped into one variant that extended multiple residues. The number of reads that support any given variant was determined for each sample. This raw variant read count per sample was normalized by the total number of reads per sample (after filtering for low expected number of errors per read, given the phred quality scores) to account for different sequencing depths. Technical replicates were combined by finding the geometric mean of variant normalized read count (shown below, ‘counts’). Enrichment was calculated for each sample by diving by the naive read count (with the same context—i.e. D2, D3, DDD). To down weight the enrichment associated with low read count, a pseudocount of 10 was added to the numerator and denominator during the enrichment calculation. The enrichment for each context is the median across the individual gates, and the enrichment overall is the median enrichment across the gates and contexts. Enrichment error is the standard deviation of the log2 enrichment values, divided by the sqrt of the number of values per variant, multiplied by 2 to make a 95% confidence interval on the mean.

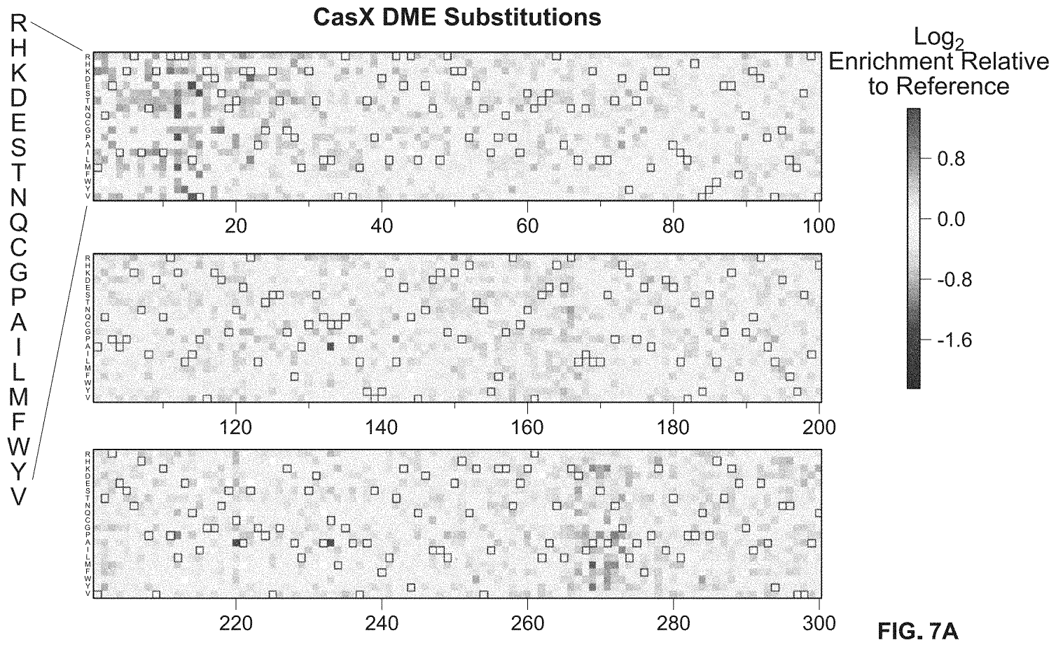

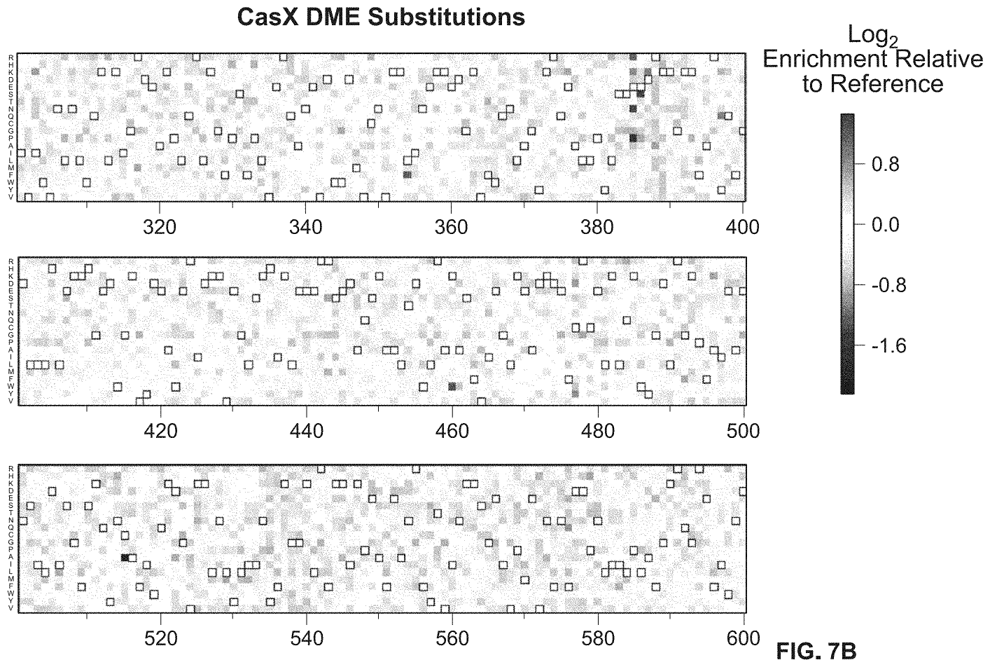

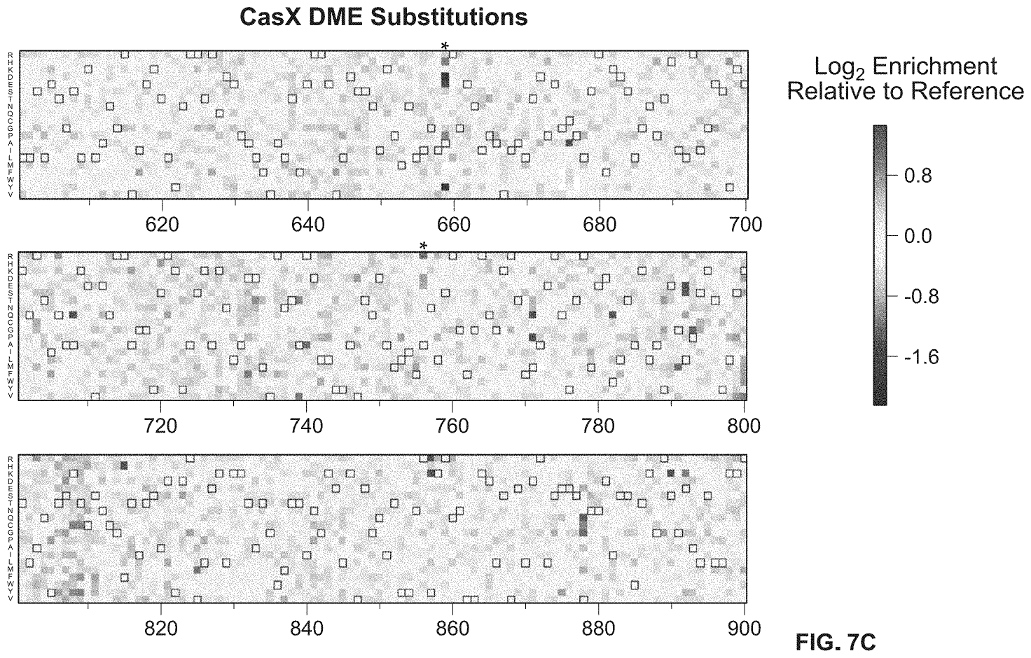

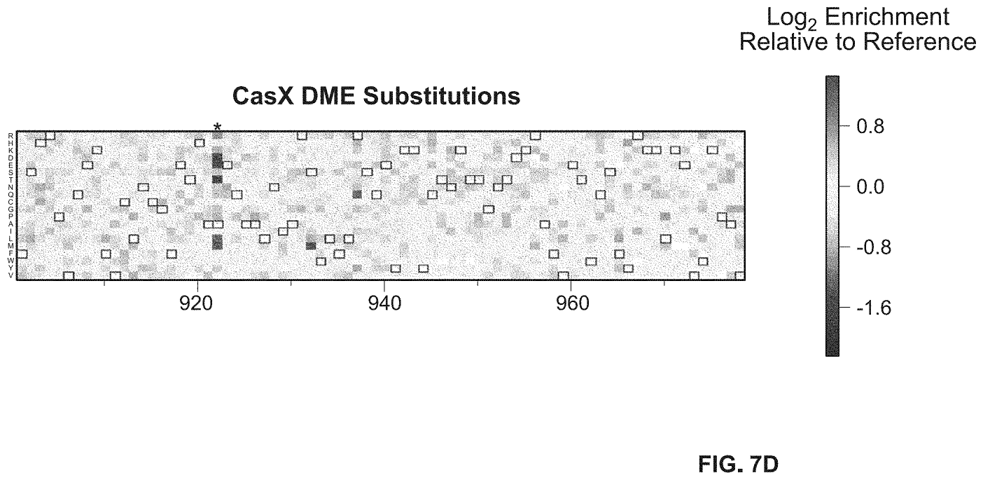

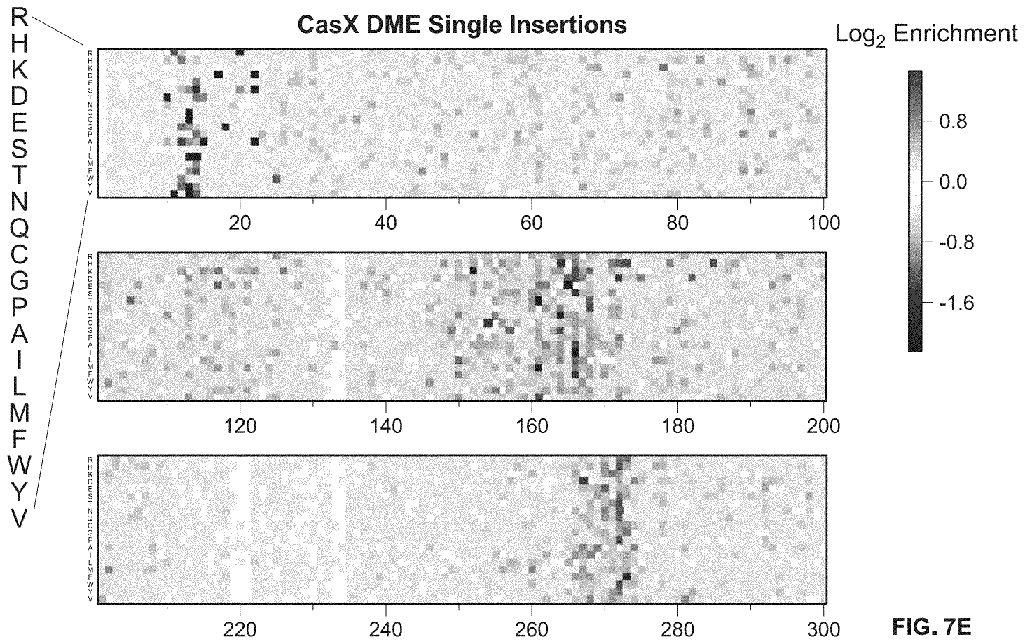

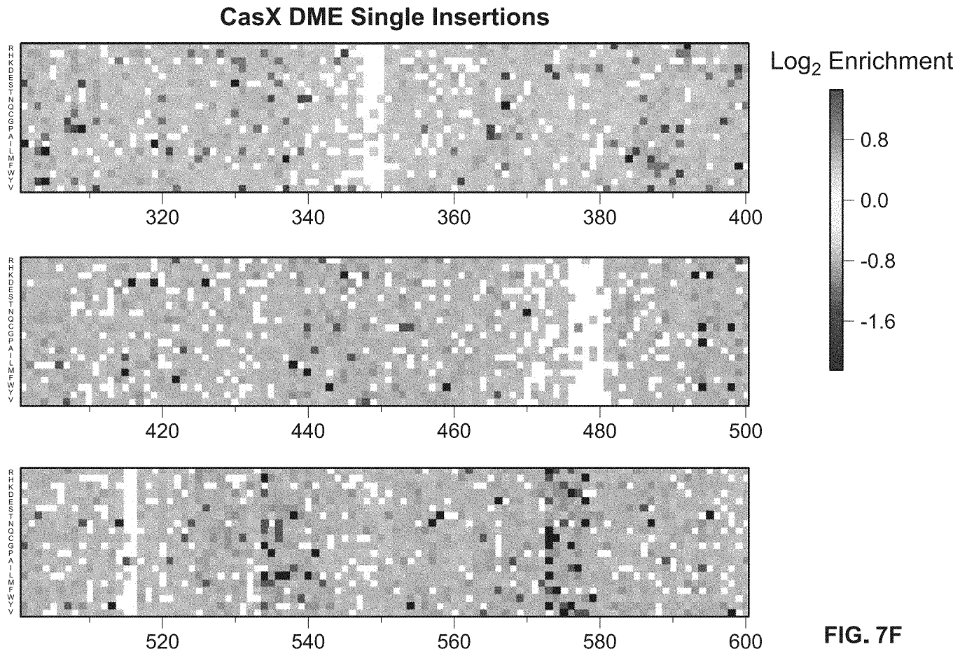

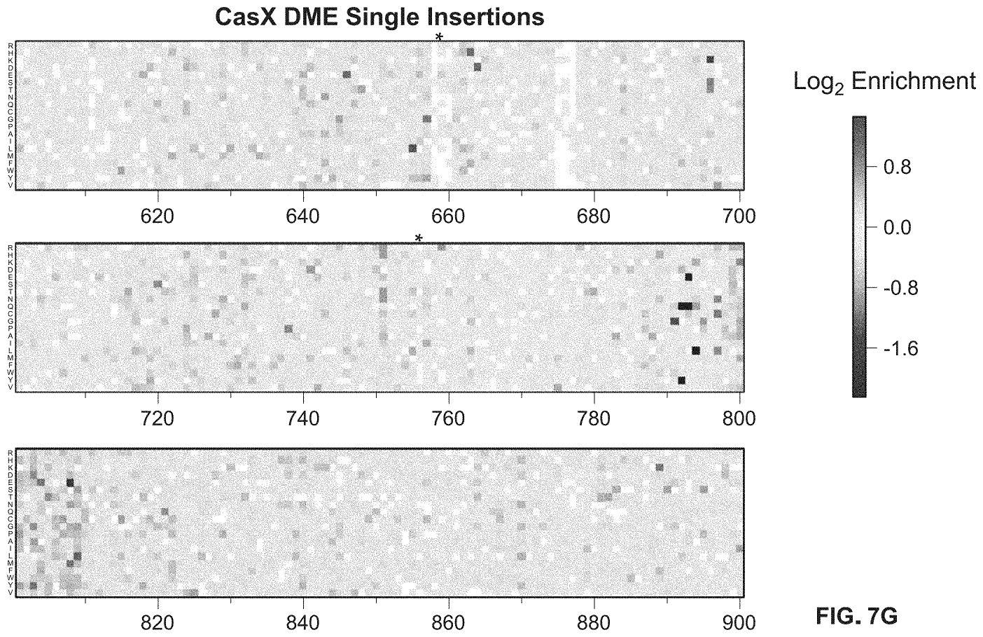

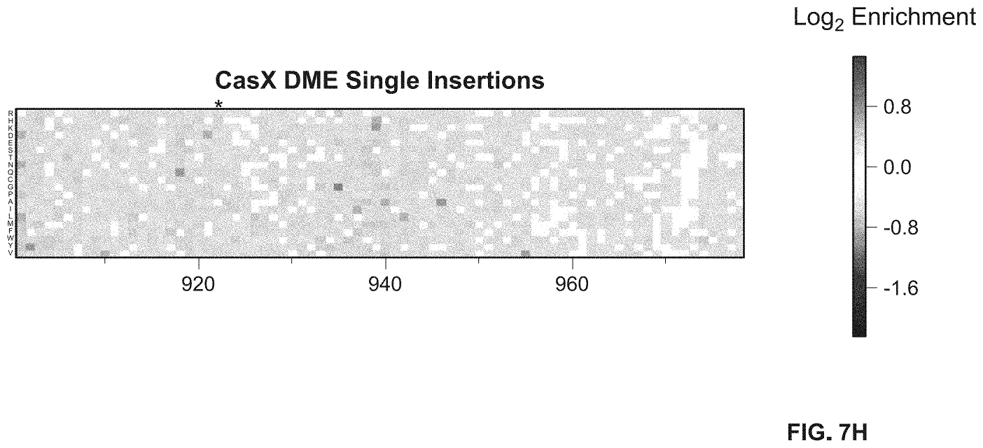

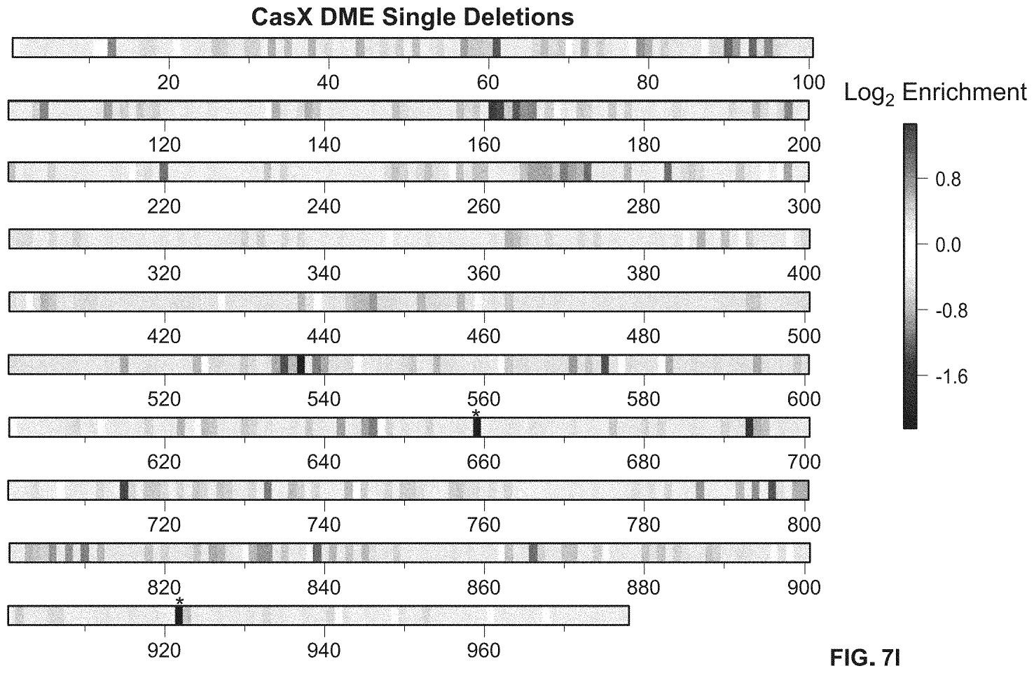

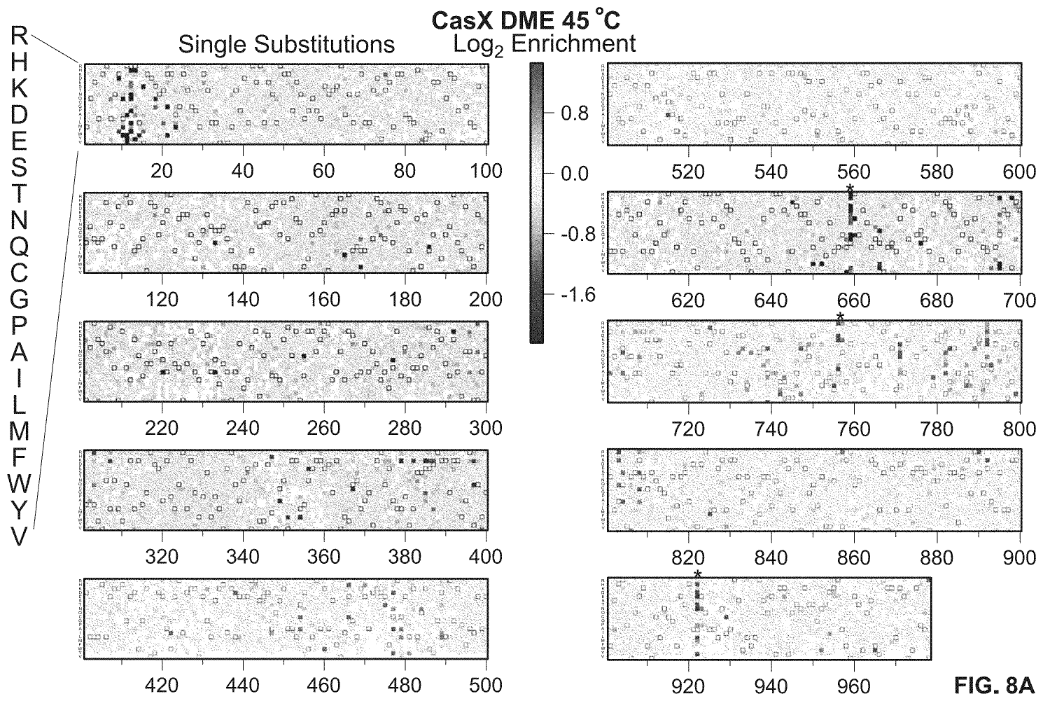

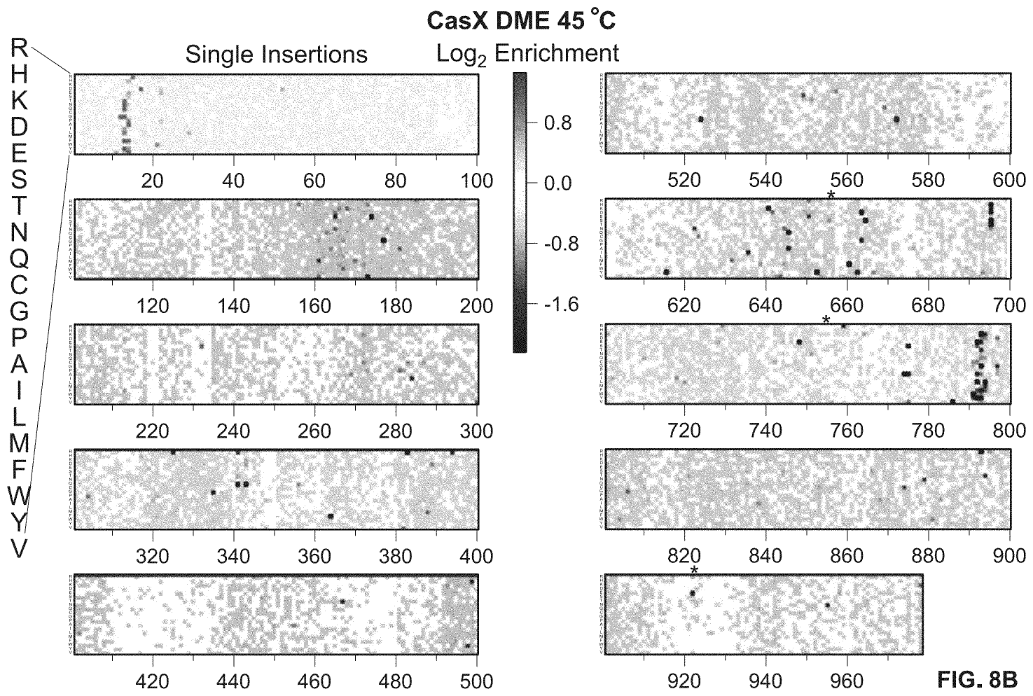

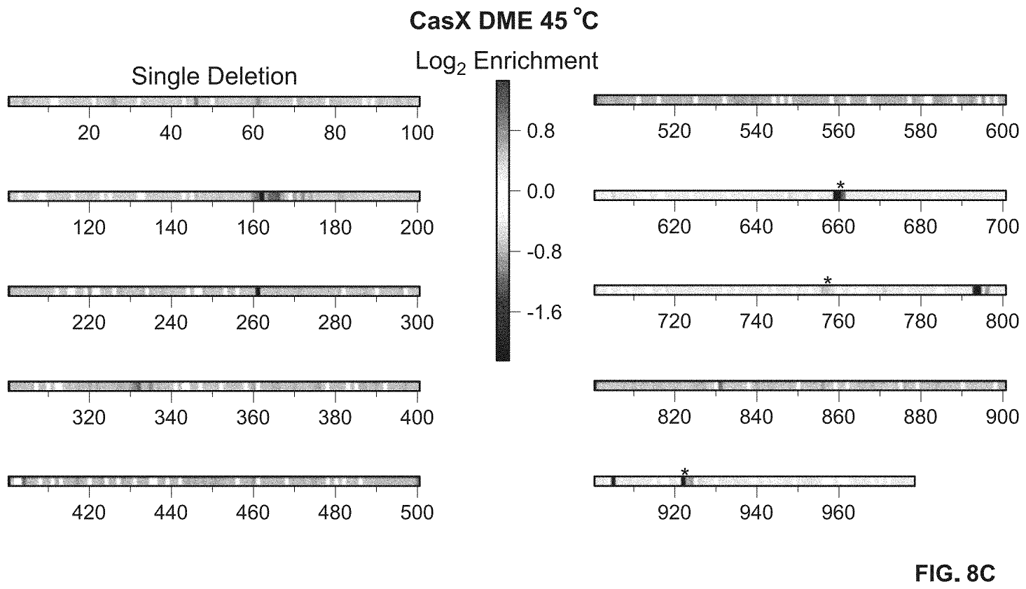

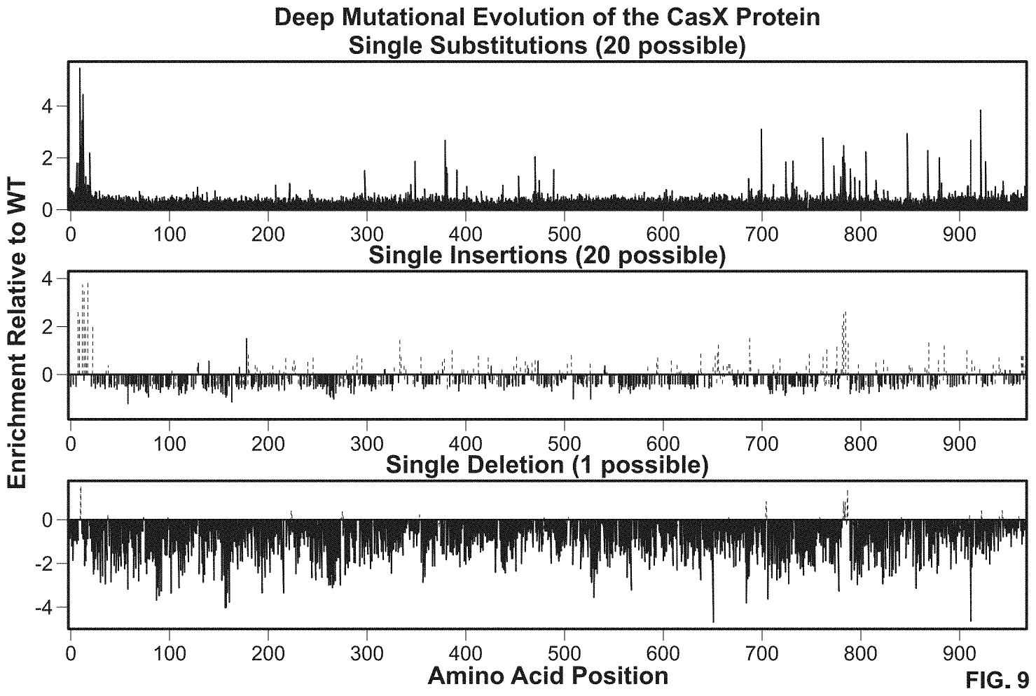

Heat maps of DME variant enrichment for each position of the CasX reference protein are shown in FIGS. 7A-7I and FIGS. 8A-8C. Fold enrichment of DME variants with single substitutions, insertions and deletions of each amino acid of the reference CasX protein of SEQ ID NO: 2 are shown. FIGS. 7A-7I and Table 28 summarize the results when the DME experiment was run at 37° C. FIGS. 8A-8C summarize the results when the same experiment was run at 45° C. A comparison of the data in FIGS. 7A-7I and FIGS. 8A-8C shows that running the same assay at two temperatures enriches for different variants. A comparison of the two temperatures thus indicates which amino acid residues and changes are important for thermostability and folding, and can be targeted to produce CasX variant proteins with improved thermostability and folding. FIG. 9 shows a survey of the comprehensive mutational landscape of all single mutations of the reference CasX protein of SEQ ID NO: 2.

TABLE 28
Fold enrichment of CasX DME variants.

| Pos. | Ref. | Alt. | Med. Enrich. | 95% CI | Pos. | Ref. | Alt. | Med. Enrich. | 95% CI |
|---|---|---|---|---|---|---|---|---|---|
| 11 | R | N | 3.123689614 | 1.666090155 | 877 | V | D | 1.738762289 | 0.688664606 |
| 13 | -- | AS | 2.772897791 | 0.812692873 | 459 | K | W | 1.696823829 | 0.67904004 |
| 13 | -- | AG | 2.740825108 | 1.138556052 | 891 | E | K | 1.6928634 | 0.819015932 |
| 12 | - | V | 2.739405927 | 1.743064315 | 9 | - | T | 1.667698181 | 0.626564384 |
| 13 | -- | TS | 2.69239793 | 1.005397595 | 19 | - | R | 1.664532235 | 0.885325268 |
| 12 | - | Y | 2.676525308 | 1.621386271 | 11 | R | P | 1.655382042 | 1.234907956 |
| 754 | FE | LA | 2.638126094 | 0.709679147 | 793 | - | L | 1.585086754 | 0.91714318 |
| 13 | - | L | 2.63160466 | 1.131924801 | 931 | S | L | 1.583295371 | 0.643295534 |
| 14 | V | S | 2.616515776 | 1.515637887 | 12 | -- | AG | 1.580094246 | 1.037517499 |
| 877 | V | G | 2.558943878 | 1.132565008 | 770 | M | P | 1.577648056 | 1.061356917 |
| 21 | - | D | 2.295527175 | 0.893253582 | 791 | L | E | 1.551380949 | 0.823309399 |
| 12 | -- | PG | 2.222956581 | 1.243693989 | 21 | - | A | 1.542633652 | 0.760237264 |
| 824 | V | M | 2.181465681 | 1.137291381 | 814 | F | H | 1.510927821 | 0.672796928 |
| 12 | - | Q | 2.102167857 | 1.396704669 | 12 | - | C | 1.506305374 | 0.730799624 |
| 13 | L | E | 2.049540302 | 0.886997965 | 791 | L | S | 1.505731571 | 0.598349327 |
| 12 | R | A | 2.046419725 | 1.229773759 | 792 | -- | AS | 1.474378912 | 0.833339427 |
| 889 | S | K | 2.030682939 | 0.721857305 | 12 | - | L | 1.46896091 | 0.783746198 |
| 791 | - | Q | 1.996189679 | 0.799796529 | 795 | T | - | 1.465811841 | 0.744738295 |
| 21 | - | S | 1.907167641 | 0.736834562 | 792 | - | Q | 1.462809015 | 0.586506727 |
| 14 | - | A | 1.89090961 | 1.25865759 | 11 | R | S | 1.459875087 | 0.740946571 |
| 11 | R | M | 1.88125645 | 0.779897343 | 11 | R | T | 1.450818176 | 0.908088492 |
| 856 | Y | R | 1.83253552 | 0.74976479 | 738 | A | V | 1.397545277 | 0.638310372 |
| 707 | A | Q | 1.830052571 | 0.555234229 | 791 | - | Y | 1.382702158 | 0.877495368 |
| 16 | - | D | 1.826796594 | 1.168291076 | 384 | E | P | 1.36783963 | 0.775382596 |
| 17 | S | G | 1.799890039 | 0.536675637 | 793 | -- | ST | 1.351743597 | 0.608183464 |
| 931 | S | M | 1.798321904 | 1.171026479 | 738 | A | T | 1.349932545 | 0.581386051 |
| 13 | L | V | 1.782912682 | 0.513630591 | 781 | W | Q | 1.342276465 | 0.719454459 |
| 11 | -- | AS | 1.782444935 | 0.75642805 | 17 | - | G | 1.340746587 | 0.878053267 |
| 856 | Y | K | 1.748619552 | 0.651026121 | 12 | -- | AS | 1.333635165 | 1.19716917 |
| 796 | -- | AS | 1.742437726 | 0.859039085 | 771 | A | Y | 1.292995852 | 0.871463205 |
| 792 | - | E | 1.290525566 | 1.195462062 | 979 | L-E[stop] | VSSK (SEQ | 1.125229136 | 0.372301096 |
| | | | | | | | ID NO: 3797) | | |
| 921 | A | M | 1.28763891 | 0.560591034 | 936 | R | Q | 1.117866436 | 0.745233062 |
| 979 | LE[stop]GS- | VSSKDL | 1.282505495 | 0.371661154 | 979 | LE[stop]GS- | VSSKDLQAS | 1.111969193 | 0.311410682 |
| | | (SEQ ID NO: | | | | PGIK (SEQ ID | N (SEQ ID | | |
| | | 3804) | | | | NO: 3279) | NO: 3813) | | |
| 770 | M | Q | 1.279910431 | 1.186538897 | 396 | Y | Q | 1.105278825 | 0.646150998 |
| 16 | -- | AG | 1.271874994 | 0.55951096 | 979 | LE[stop]GSP | VSSKDL | 1.104849849 | 0.260693612 |
| | | | | | | | (SEQ ID NO: | | |
| | | | | | | | 3804) | | |
| 384 | E | N | 1.247124467 | 0.607911368 | 353 | L | F | 1.103922948 | 0.510520582 |
| 979 | L- | VS | 1.239823793 | 0.315337927 | 979 | LE[stop]GS- | VSSKDLQA | 1.100880851 | 0.345695892 |
| | | | | | | PG (SEQ ID | (SEQ ID NO: | | |
| | | | | | | NO: 3251) | 3810) | | |
| 979 | LE[stop] | VSS | 1.233215135 | 0.36262523 | 697 | Y | H | 1.097977697 | 0.419010874 |
| 658 | --D | APG | 1.220851584 | 0.979760686 | 796 | -- | PG | 1.095168865 | 0.816765224 |
| 979 | L-E | VSS | 1.21568584 | 0.37106558 | 4 | -- | TS | 1.088089915 | 0.693109756 |
| 385 | E | S | 1.210243487 | 0.826999735 | 10 | R | K | 1.085472062 | 0.382234839 |
| 979 | LE[stop]GS- | VSSKDLQAS | 1.208612972 | 0.286427519 | 790 | G | M | 1.066566819 | 0.686227232 |
| | PGIK (SEQ ID | NK (SEQ ID | | | | | | | |
| | NO: | NO: 3814) | | | | | | | |
| | 3279)[stop] | | | | | | | | |
| 793 | -- | SA | 1.192367811 | 0.72089465 | 921 | A | K | 1.056315246 | 0.70226115 |
| 739 | R | A | 1.188987234 | 0.611670208 | 696 | - | R | 1.049001055 | 0.880941583 |
| 795 | -- | AS | 1.183930928 | 0.90542554 | 9 | I | L | 1.039309233 | 0.528320595 |
| 979 | LE[stop]GS-P | VSSKDLQ | 1.180100725 | 0.35995062 | 979 | LE[stop]GSPG | VSSKDLQAS | 1.037884742 | 0.299531766 |
| | | (SEQ ID NO: | | | | IK (SEQ ID | NK (SEQ ID | | |
| | | 3809) | | | | NO: | NO: 3814) | | |
| | | | | | | 3279)[stop]N | | | |
| 977 | V | K | 1.17977084 | 0.720108501 | 13 | - | S | 1.031062599 | 0.727357338 |
| 658 | --D | AAS | 1.173300666 | 0.50353561 | 384 | E | R | 1.028117481 | 0.683537724 |
| 14 | -- | TS | 1.173232132 | 0.700156049 | 21 | K | D | 1.019445543 | 0.748518701 |
| 10 | - | V | 1.164019233 | 1.085055677 | 978 | [stop] | G | 1.016498062 | 0.514955543 |
| 375 | E | K | 1.163948709 | 0.891802018 | 979 | L-E[stop]G | VSSKD (SEQ | 1.016126075 | 0.353515679 |
| | | | | | | | ID NO: 3800) | | |
| 795 | -- | AG | 1.14629929 | 0.481029275 | 10 | R | N | 1.010184099 | 0.846798556 |
| 979 | LE[stop]GSPG | VSSKDLQ | 1.143633475 | 0.340695621 | 794 | -- | PG | 1.00924007 | 0.987312969 |
| | (SEQ ID NO: | (SEQ ID NO: | | | | | | | |
| | 3251) | 3809) | | | | | | | |
| 979 | LE | VS | 1.142516835 | 0.386398408 | 741 | L | W | 0.851844349 | 0.594072278 |
| 877 | V | Q | 1.141917178 | 0.655790093 | 24 | - | W | 0.835220929 | 0.745009807 |
| 791 | L | Q | 1.004388299 | 0.361910793 | 755 | E | [stop] | 0.833955657 | 0.31600491 |
| 792 | P | G | 1.002325281 | 0.805296973 | 928 | I | T | 0.832425124 | 0.307759846 |
| 877 | V | C | 0.995089773 | 0.566724231 | 979 | LE[stop]GS- | VSSKDLQAS | 0.822335062 | 0.317179456 |
| | | | | | | PGI (SEQ ID | (SEQ ID NO: | | |
| | | | | | | NO: 3278) | 3812) | | |
| 476 | C | Y | 0.984546648 | 0.686487573 | 781 | W | K | 0.810589018 | 0.686153856 |
| 19 | -- | PG | 0.984071689 | 0.738694244 | 791 | L | R | 0.806201856 | 0.611654466 |
| 979 | LE[stop]GSPG | VSSKDLQA | 0.972011014 | 0.292930615 | 979 | LE[stop]GSPG | VSSKDLQAS | 0.80600706 | 0.220866187 |
| | I (SEQ ID NO: | (SEQ ID NO: | | | | IK (SEQ ID | N (SEQ ID | | |
| | 3278) | 3810) | | | | NO: | NO: 3813) | | |
| | | | | | | 3279)[stop] | | | |
| 752 | L | P | 0.971338521 | 0.459371253 | 711 | E | Q | 0.793874739 | 0.38732268 |
| 12 | R | C | 0.969988229 | 0.745286116 | 703 | T | N | 0.791134752 | 0.735228799 |
| 12 | R | Y | 0.962112567 | 0.714384629 | 793 | S | - | 0.7821232 | 0.523699668 |
| 979 | LE[stop]GSPG | VSSKDLQAS | 0.960035296 | 0.298173201 | 385 | E | K | 0.781091846 | 0.579724424 |
| | IK (SEQ ID | (SEQ ID NO: | | | | | | | |
| | NO: 3279) | 3812) | | | | | | | |
| 18 | -- | PG | 0.952532997 | 0.782330584 | 955 | R | M | 0.780963169 | 0.340474646 |
| 778 | M | I | 0.945963409 | 0.345538178 | 469 | - | N | 0.775656135 | 0.541879732 |
| 798 | S | P | 0.942103893 | 0.470224487 | 788 | Y | T | 0.770125047 | 0.581859138 |
| 16 | D | G | 0.941159649 | 0.341870864 | 705 | Q | R | 0.76633283 | 0.261069709 |
| 22 | A | Q | 0.937573643 | 0.676316271 | 9 | -- | TS | 0.763723778 | 0.674640849 |
| 754 | FE | IA | 0.935796963 | 0.660936674 | 979 | LE[stop]GS | VSSKD (SEQ | 0.761764547 | 0.205465156 |
| | | | | | | | ID NO: 3800) | | |
| 1 | Q | K | 0.935474248 | 0.373656765 | 715 | A | K | 0.761122086 | 0.540516283 |
| 14 | V | F | 0.932689058 | 0.742246472 | 384 | E | K | 0.760859162 | 0.22641046 |
| 8 | K | I | 0.928472117 | 0.521050669 | 591 | QG | R- | 0.757963418 | 0.374903235 |
| 384 | E | G | 0.920571639 | 0.452302777 | 316 | R | M | 0.757086682 | 0.310302995 |
| 732 | D | T | 0.912254061 | 0.759438627 | 770 | M | T | 0.753193128 | 0.319236781 |
| 658 | D | Y | 0.894131769 | 0.312165116 | 384 | E | Q | 0.752976137 | 0.602376709 |
| 211 | L | P | 0.887315174 | 0.318877781 | 17 | S | E | 0.752400908 | 0.414988963 |
| 14 | V | A | 0.885138345 | 0.699864156 | 755 | E | D | 0.74863141 | 0.212934852 |
| 979 | LE[stop]G | V--S | 0.884897395 | 0.252782429 | 12 | R | - | 0.743504623 | 0.648509511 |
| 13 | - | F | 0.883212774 | 0.713984249 | 938 | Q | E | 0.741570425 | 0.469451701 |
| 979 | LE[stop]G | VSSK (SEQ | 0.881127427 | 0.417135617 | 657 | I | V | 0.73806027 | 0.256874713 |
| | ID NO: 3797) | | | | | | | | |
| 386 | D | K | 0.879045429 | 0.728272074 | 656 | G | C | 0.659813316 | 0.293973226 |
| 5 | R | I | 0.871114116 | 0.317513506 | 4 | K | N | 0.656251908 | 0.302190904 |
| 660 | -- | AS | 0.862493953 | 0.798632847 | 774 | Q | E | 0.654737733 | 0.134116674 |
| 877 | V | M | 0.855677916 | 0.267740831 | -1 | S | C | 0.652333059 | 0.118222939 |
| -1 | S | T | 0.735179004 | 0.144429929 | 21 | -- | AS | 0.651563705 | 0.48650799 |
| 2 | E | [stop] | 0.734071396 | 0.323713248 | 185 | L | P | 0.649897837 | 0.225081568 |
| 384 | E | A | 0.733775595 | 0.660142332 | 38 | P | T | 0.648698083 | 0.350485275 |
| 891 | E | Y | 0.733458673 | 0.465192765 | 936 | R | H | 0.648045448 | 0.423309347 |
| 643 | V | F | 0.732765961 | 0.577614171 | 813 | G | C | 0.644003475 | 0.310838653 |
| 796 | - | C | 0.732364738 | 0.485790322 | 786 | L | M | 0.643153738 | 0.314936636 |
| 280 | L | M | 0.731787266 | 0.258239226 | 942 | K | N | 0.639528926 | 0.249553292 |
| 695 | - | K | 0.730902961 | 0.509205112 | 293 | Y | H | 0.636816244 | 0.207205991 |
| 343 | W | L | 0.725824372 | 0.292120452 | 542 | F | L | 0.635949082 | 0.181128276 |
| 3 | ------ | IKRINK (SEQ | 0.721338414 | 0.470264314 | 303 | W | L | 0.635588216 | 0.261903568 |
| | | ID NO: 3475) | | | | | | | |
| 732 | D | N | 0.71945188 | 0.416870981 | 979 | LE | V[stop] | 0.635165807 | 0.329009453 |
| 687 | --- | PTH | 0.716433371 | 0.159856315 | 578 | P | H | 0.634392073 | 0.324298942 |
| 176 | A | D | 0.71514177 | 0.206626688 | 687 | -- | PT | 0.633217575 | 0.355316701 |
| 485 | W | L | 0.713411462 | 0.238105577 | 886 | K | N | 0.632562679 | 0.231080349 |
| 22 | A | D | 0.710738042 | 0.32510753 | 20 | K | R | 0.632186797 | 0.237509121 |
| 193 | L | P | 0.709349304 | 0.242633498 | 248 | L | P | 0.631068881 | 0.180279623 |
| 899 | R | M | 0.707875506 | 0.298429738 | 18 | N | S | 0.630660766 | 0.266585824 |
| 886 | KG | R- | 0.706803824 | 0.286241441 | 836 | M | V | 0.630065132 | 0.266534124 |
| 796 | -- | TS | 0.697218521 | 0.492426198 | 116 | K | N | 0.629540403 | 0.234219411 |
| 329 | P | H | 0.696817542 | 0.314817482 | 847 | EG | GA | 0.628295048 | 0.299740787 |
| 273 | L | P | 0.696199602 | 0.349703999 | 912 | L | P | 0.627137425 | 0.187179246 |
| 31 | L | M | 0.696080627 | 0.331245769 | 92 | P | H | 0.626243107 | 0.350245614 |
| 645 | - | E | 0.692307595 | 0.590013131 | 299 | Q | K | 0.623386276 | 0.302029469 |
| 9 | I | Y | 0.689813642 | 0.667593375 | 707 | A | T | 0.622086487 | 0.275515174 |
| 9 | 1 | N | 0.688953393 | 0.257809633 | 669 | L | M | 0.620453868 | 0.351072046 |
| 919 | H | R | 0.688781806 | 0.363439859 | 789 | E | D | 0.617920878 | 0.216264385 |
| 687 | P | H | 0.684782236 | 0.310607479 | 916 | F | S | 0.617302977 | 0.309372822 |
| 332 | P | H | 0.672484781 | 0.326219913 | 55 | P | li | 0.616365993 | 0.329695842 |
| 796 | - | N | 0.672333697 | 0.64437503 | 936 | R | G | 0.615282844 | 0.189389227 |
| 421 | W | L | 0.667702097 | 0.291970479 | 595 | F | L | 0.615176885 | 0.154670433 |
| 875 | E | [stop] | 0.66617872 | 0.287006304 | 0 | M | 1 | 0.612039515 | 0.303853593 |
| 378 | L | K | 0.664474618 | 0.393361359 | 925 | A | P | 0.581907283 | 0.186614282 |
| 891 | E | Q | 0.663650921 | 0.312291932 | 659 | R | L | 0.580864225 | 0.319384189 |
| 926 | L | M | 0.661737644 | 0.525550321 | 306 | L | P | 0.578183307 | 0.210431982 |
| 381 | L | R | 0.609889042 | 0.420808291 | 676 | P | Q | 0.577757554 | 0.308473522 |
| 945 | T | A | 0.609683347 | 0.258353939 | 877 | V | E | 0.57724394 | 0.294796776 |
| 389 | K | N | 0.609647876 | 0.274048697 | 19 | T | A | 0.576889973 | 0.198407278 |
| 755 | E | G | 0.607714844 | 0.078377344 | 14 | V | D | 0.574902804 | 0.437270334 |
| 559 | I | M | 0.606040482 | 0.27336203 | 887 | G | Q | 0.574717855 | 0.519529758 |
| 825 | L | P | 0.604240507 | 0.192490062 | 935 | L | V | 0.573813105 | 0.185021716 |
| 733 | M | T | 0.603960776 | 0.340233556 | 961 | W | L | 0.573698555 | 0.253700288 |
| 664 | P | T | 0.60370266 | 0.234348448 | 23 | -- | GP | 0.572198674 | 0.570313308 |
| 10 | R | T | 0.602483957 | 0.372156893 | 541 | R | L | 0.571508027 | 0.254421711 |
| 964 | F | L | 0.60175279 | 0.17004436 | 288 | E | D | 0.571482463 | 0.24542675 |
| 911 | C | S | 0.601303891 | 0.279730674 | 742 | L | V | 0.570384839 | 0.3027928 |
| 788 | Y | G | 0.600935917 | 0.580949772 | 931 | S | T | 0.570369019 | 0.120673525 |
| 447 | Q | K | 0.600543047 | 0.297568309 | 623 | ------- | RRTRQDE | 0.569913903 | 0.141118873 |
| | | | | | | | (SEQ ID NO: | | |
| | | | | | | | 3684) | | |
| 13 | L | P | 0.599989903 | 0.236688663 | 27 | P | H | 0.569605452 | 0.285015385 |
| 193 | L | M | 0.599332216 | 0.309308194 | 28 | M | T | 0.56885021 | 0.216863369 |
| 114 | P | H | 0.599262194 | 0.344450733 | 907 | E | [stop] | 0.567613159 | 0.345163987 |
| 660 | G | R | 0.599221963 | 0.319640645 | 577 | D | Y | 0.567493308 | 0.253952459 |
| 894 | S | T | 0.599084973 | 0.166490359 | 672 | P | H | 0.566921749 | 0.31335168 |
| 904 | P | H | 0.59783828 | 0.349499416 | 669 | L | P | 0.564276636 | 0.224594167 |
| 782 | L | T | 0.595786463 | 0.513346845 | 52 | E | D | 0.564250133 | 0.246311739 |
| 944 | Q | K | 0.595243666 | 0.351818545 | 46 | N | T | 0.563094073 | 0.208662987 |
| 207 | P | H | 0.595218482 | 0.277632613 | 5 | R | G | 0.560139309 | 0.15069426 |
| 151 | H | N | 0.595188624 | 0.277503327 | 912 | L | V | 0.559515875 | 0.111973397 |
| 495 | A | K | 0.594637604 | 0.315764586 | 40 | L | M | 0.558605774 | 0.239058063 |
| -1 | S | P | 0.594582952 | 0.377333364 | 923 | Q | [stop] | 0.558515774 | 0.34688202 |
| 480 | L | E | 0.594055289 | 0.432259346 | 979 | L- E[stop]G | VSSKE (SEQ | 0.557263947 | 0.22994802 |
| | | | | | | | ID NO: 3826) | | |
| 469 | E | A | 0.594025118 | 0.30338267 | 41 | R | T | 0.555902565 | 0.199937528 |
| 11 | R | G | 0.59320688 | 0.163279008 | 179 | E | [stop] | 0.555817911 | 0.245362937 |
| 85 | W | L | 0.591691074 | 0.2708118 | 344 | W | L | 0.555474112 | 0.286390208 |
| 15 | K | E | 0.587925122 | 0.149546484 | 703 | T | R | 0.53396819 | 0.160757401 |
| 755 | E | K | 0.586636571 | 0.217538569 | 962 | Q | E | 0.533896042 | 0.302336405 |
| 337 | Q | R | 0.585098232 | 0.172195554 | 764 | Q | H | 0.53385913 | 0.24340782 |
| 877 | V | A | 0.584567684 | 0.258968272 | 793 | S | T | 0.533306619 | 0.17379091 |
| 793 | -- | TS | 0.583269098 | 0.45091329 | 6 | I | M | 0.533192185 | 0.188523563 |
| 670 | I | R | 0.582033902 | 0.112618756 | 467 | L | P | 0.533022246 | 0.179464215 |
| 63 | R | M | 0.554978749 | 0.336590825 | 244 | Q | [stop] | 0.532045714 | 0.262393061 |
| 1 | Q | R | 0.554755158 | 0.207724233 | 8 | K | N | 0.531704561 | 0.294399975 |
| 9 | I | V | 0.554053334 | 0.219348804 | 508 | F | V | 0.529042378 | 0.192146822 |
| 914 | C | [stop] | 0.552658801 | 0.347714953 | 665 | A | P | 0.529013767 | 0.174049723 |
| 836 | M | I | 0.551813626 | 0.180327214 | 46 | NL | T[stop] | 0.529006897 | 0.272198259 |
| 856 | Y | H | 0.549262192 | 0.369311354 | 3 | I | V | 0.528916598 | 0.14506718 |
| 620 | L | M | 0.548957556 | 0.322210662 | 518 | W | S | 0.528332889 | 0.199792834 |
| 926 | L | P | 0.547714601 | 0.450095044 | 792 | P | A | 0.528028079 | 0.112407207 |
| 377 | L | P | 0.546553821 | 0.20366425 | 13 | L | A | 0.526728857 | 0.318983292 |
| 920 | A | S | 0.545992524 | 0.484867291 | 56 | Q | K | 0.526387006 | 0.188452852 |
| 961 | W | [stop] | 0.544371204 | 0.244581668 | 878 | N | S | 0.526073971 | 0.27887921 |
| 746 | V | G | 0.543151726 | 0.512718498 | 213 | Q | E | 0.525578421 | 0.16885346 |
| 554 | --- | RFY | 0.542549772 | 0.20487223 | 748 | Q | H | 0.525406412 | 0.200108279 |
| 664 | P | H | 0.542466431 | 0.281534858 | 15 | K | N | 0.525094369 | 0.273038164 |
| 5 | R | [stop] | 0.541304946 | 0.166704906 | 954 | K | N | 0.524763966 | 0.208680978 |
| 803 | Q | K | 0.540975244 | 0.291121648 | 835 | W | L | 0.524725836 | 0.26540236 |
| 652 | M | I | 0.540953074 | 0.217563311 | 847 | E | D | 0.524019387 | 0.23897504 |
| 326 | KG | R- | 0.540593574 | 0.402287668 | 608 | L | M | 0.523890883 | 0.248052068 |
| 789 | E | [stop] | 0.540122225 | 0.236046287 | 932 | W | R | 0.523129128 | 0.299781077 |
| 889 | S | L | 0.539927241 | 0.375365013 | 21 | K | N | 0.522953217 | 0.250998038 |
| 10 | R | I | 0.539433301 | 0.326816988 | 790 | G | [stop] | 0.5229473 | 0.262740975 |
| 725 | K | N | 0.539088606 | 0.178127049 | 707 | A | D | 0.522560362 | 0.214610237 |
| 603 | L | P | 0.538897648 | 0.229282796 | 954 | K | V | 0.522546614 | 0.349200627 |
| 15 | K | R | 0.538786311 | 0.154390287 | 952 | T | A | 0.521534511 | 0.149679645 |
| 541 | R | G | 0.537572295 | 0.133876643 | 892 | A | D | 0.521298872 | 0.228218092 |
| 632 | L | M | 0.537440995 | 0.246129141 | 847 | ------- | EGQITYY | 0.521149636 | 0.115331328 |
| | | | | | | | (SEQ ID NO: | | |
| | | | | | | | 3388) | | |
| 665 | A | S | 0.536996011 | 0.286216687 | 7 | N | I | 0.521103862 | 0.202836314 |
| 650 | K | E | 0.536939626 | 0.139863469 | 917 | E | K | 0.509268127 | 0.386629094 |
| 932 | W | L | 0.536075206 | 0.314946873 | 12 | R | I | 0.509210198 | 0.267908359 |
| 684 | L | M | 0.535519584 | 0.338883641 | 326 | K | N | 0.508325806 | 0.277854988 |
| 918 | T | R | 0.535067274 | 0.304580877 | 802 | A | W | 0.507146644 | 0.398619961 |
| 10 | R | G | 0.534873359 | 0.3557865 | 627 | Q | H | 0.506946344 | 0.17779761 |
| 575 | F | L | 0.534865272 | 0.139851134 | 705 | Q | K | 0.506601342 | 0.205329495 |
| 737 | T | G | 0.534759369 | 0.303617666 | 935 | L | P | 0.505173269 | 0.279127846 |
| 907 | E | G | 0.534688762 | 0.240107856 | 636 | L | P | 0.504912592 | 0.279575261 |
| 702 | R | M | 0.520743818 | 0.247227864 | 378 | L | V | 0.504856105 | 0.146721248 |
| 901 | S | G | 0.520379757 | 0.143482219 | 770 | M | I | 0.502407214 | 0.148647414 |
| 560 | N | H | 0.519240936 | 0.286066696 | 302 | I | T | 0.502263164 | 0.328365742 |
| 350 | V | M | 0.518159753 | 0.277778553 | 584 | P | H | 0.501836401 | 0.188263444 |
| 535 | F | L | 0.518099748 | 0.153008763 | 962 | Q | H | 0.501557133 | 0.21210836 |
| 512 | Y | H | 0.517168474 | 0.223506594 | 909 | F | L | 0.501216251 | 0.397907118 |
| 278 | 1 | M | 0.516794992 | 0.238648894 | 522 | G | C | 0.50035512 | 0.232143601 |
| 746 | V | A | 0.51672383 | 0.202625874 | 233 | M | I | 0.500272986 | 0.246898577 |
| 664 | P | R | 0.516702968 | 0.252959416 | 284 | P | R | 0.499965267 | 0.18413971 |
| -1 | S | A | 0.516689693 | 0.142459137 | 639 | E | D | 0.499845638 | 0.16815712 |
| 298 | A | D | 0.51645727 | 0.257163483 | 351 | K | E | 0.49917291 | 0.274793088 |
| 361 | G | C | 0.515521808 | 0.242033529 | 12 | R | S | 0.498984129 | 0.193129295 |
| 424 | 1 | V | 0.515355817 | 0.185117148 | 920 | A | V | 0.498509984 | 0.394258252 |
| 907 | E | D | 0.514835248 | 0.277377403 | 709 | E | [stop] | 0.498173203 | 0.222297538 |
| 923 | Q | E | 0.514826301 | 0.324456465 | 443 | S | H | 0.498010803 | 0.445232627 |
| 413 | W | L | 0.514728329 | 0.241932097 | 27 | P | L | 0.497724007 | 0.373177387 |
| 748 | Q | R | 0.514571576 | 0.240563892 | 849 | Q | K | 0.497661989 | 0.259123161 |
| 591 | Q | H | 0.514415886 | 0.331792035 | 793 | - | Q | 0.497102388 | 0.47673495 |
| 1 | Q | E | 0.514404075 | 0.263908964 | 750 | A | G | 0.496799617 | 0.243940432 |
| 171 | P | T | 0.513803013 | 0.237477165 | 26 | G | C | 0.496365725 | 0.228107532 |
| 544 | K | R | 0.512919851 | 0.163480182 | 706 | A | D | 0.494947511 | 0.225683587 |
| 677 | ------- | LSRFKD | 0.511837147 | 0.194279796 | 431 | L | P | 0.494543065 | 0.192514906 |
| | | (SEQ ID NO: | | | | | | | |
| | | 3577) | | | | | | | |
| 377 | L | M | 0.511718619 | 0.274965484 | 13 | LV | AS | 0.494489513 | 0.367074627 |
| 1 | Q | H | 0.511496323 | 0.29357307 | 0 | M | V | 0.49405414 | 0.206071479 |
| 202 | R | M | 0.511365875 | 0.303187834 | 614 | R | I | 0.494053835 | 0.209299062 |
| 422 | E | [stop] | 0.511043687 | 0.224103239 | 248 | L | M | 0.49299868 | 0.24880607 |
| 922 | E | [stop] | 0.510570886 | 0.450135707 | 81 | L | M | 0.492127571 | 0.369172442 |
| 407 | ------- | KKHGED | 0.510425363 | 0.211479415 | | | | | |
| | | (SEQ ID NO: | | | | | | | |
| | | 3500) | | | | | | | |
| 8 | K | A | 0.510125467 | 0.417426274 | 921 | D | Y | 0.479522102 | 0.330930172 |
| 300 | I | M | 0.510084254 | 0.178542003 | 17 | S | R | 0.479410291 | 0.242870401 |
| 668 | A | P | 0.509985424 | 0.202934866 | 23 | G | C | 0.47738757 | 0.286426817 |
| 418 | - | D | 0.49144742 | 0.21486801 | 892 | A | G | 0.477302415 | 0.253000116 |
| 914 | C | R | 0.490784001 | 0.353820866 | 832 | A | T | 0.47606534 | 0.23451824 |
| 3 | I | S | 0.490305334 | 0.219289736 | 421 | W | [stop] | 0.475666945 | 0.216973062 |
| 781 | W | L | 0.490256264 | 0.225567162 | 316 | R | S | 0.47464939 | 0.264534919 |
| 234 | G | [stop] | 0.489800943 | 0.231905474 | 681 | K | N | 0.474468269 | 0.192816933 |
| 369 | A | V | 0.489746571 | 0.142680124 | 22 | A | V | 0.474221933 | 0.206217506 |
| 685 | G | C | 0.48966455 | 0.174412352 | 691 | L | M | 0.473867575 | 0.189071763 |
| 498 | A | S | 0.489397172 | 0.173872708 | 95 | L | V | 0.473859579 | 0.188485586 |
| 746 | V | D | 0.488692506 | 0.484120982 | 827 | K | N | 0.47365473 | 0.198868181 |
| 666 | -- | AG | 0.488446913 | 0.383322789 | 858 | R | M | 0.473407136 | 0.257236194 |
| 309 | W | L | 0.487964134 | 0.209151088 | 519 | Q | P | 0.472315609 | 0.224391717 |
| 979 | ---- | VSSK (SEQ | 0.486810051 | 0.287650542 | 95 | L | P | 0.471361064 | 0.162277972 |
| | | ID NO: 3797) | | | | | | | |
| 27 | P | R | 0.486771244 | 0.185539954 | 976 | A | T | 0.470889659 | 0.109031 |
| 583 | L | M | 0.486474099 | 0.232216764 | 782 | L | I | 0.470558203 | 0.125178365 |
| 760 | G | R | 0.485722591 | 0.195838563 | 723 | A | S | 0.469929973 | 0.218713854 |
| 596 | I | T | 0.485474246 | 0.130718203 | 24 | K | R | 0.469399175 | 0.236250784 |
| 189 | G | [stop] | 0.484957086 | 0.271997616 | 748 | Q | E | 0.46890075 | 0.291020418 |
| 884 | W | L | 0.48469466 | 0.210361106 | 686 | --- | NPT | 0.468711675 | 0.157459195 |
| 162 | E | [stop] | 0.484515492 | 0.270313618 | 1 | Q | L | 0.468380179 | 0.341181409 |
| 405 | L | P | 0.484058533 | 0.143471721 | 466 | G | V | 0.467982153 | 0.207162352 |
| 815 | T | A | 0.483688268 | 0.140346764 | 346 | --- | MVC | 0.467747954 | 0.140593808 |
| 875 | E | D | 0.483680843 | 0.230122106 | 746 | V | L | 0.467699466 | 0.162488099 |
| 703 | T | K | 0.483561705 | 0.243688021 | 101 | Q | K | 0.467562845 | 0.263058522 |
| 35 | V | A | 0.48268809 | 0.163074127 | 99 | V | L | 0.467355555 | 0.098627209 |
| 320 | K | E | 0.482629615 | 0.202594011 | 354 | I | M | 0.46704321 | 0.243813968 |
| 203 | E | D | 0.482289135 | 0.173584261 | 826 | E | [stop] | 0.466802563 | 0.164892155 |
| 202 | R | S | 0.482184999 | 0.1640178 | 150 | P | L | 0.466773068 | 0.200507693 |
| 613 | G | C | 0.482001189 | 0.220237462 | 476 | C | R | 0.466682009 | 0.123054893 |
| 220 | A | P | 0.481251117 | 0.159715468 | 38 | P | H | 0.466309116 | 0.291701454 |
| 920 | A | G | 0.481026982 | 0.321704418 | 120 | E | [stop] | 0.465867266 | 0.21730484 |
| 874 | E | Q | 0.480905869 | 0.250463545 | 370 | G | R | 0.465477814 | 0.252126933 |
| 192 | A | G | 0.480770514 | 0.112319124 | 7 | N | K | 0.465102103 | 0.221573061 |
| 578 | P | T | 0.48002354 | 0.203348553 | 920 | A | P | 0.45449471 | 0.288443793 |
| 515 | A | P | 0.480000762 | 0.142980394 | 701 | Q | H | 0.453812486 | 0.146230302 |
| 55 | P | T | 0.465075846 | 0.236340763 | 891 | E | [stop] | 0.453785945 | 0.233457013 |
| 681 | K | E | 0.464515385 | 0.142005053 | 133 | C | W | 0.453639333 | 0.137405208 |
| 781 | W | C | 0.464433122 | 0.295451154 | 370 | G | V | 0.453597184 | 0.202403506 |
| 946 | N | D | 0.463522655 | 0.373105851 | 548 | E | D | 0.453077345 | 0.109679349 |
| 368 | L | M | 0.463023353 | 0.266615533 | 689 | H | D | 0.453055551 | 0.09160837 |
| 0 | M | T | 0.462868938 | 0.232012879 | 931 | S | R | 0.45302365 | 0.382294772 |
| 737 | T | A | 0.462760296 | 0.301960654 | 133 | C | [stop] | 0.452586533 | 0.10138833 |
| 847 | ---- | EGQI (SEQ | 0.462759431 | 0.219565444 | 868 | E | [stop] | 0.452282618 | 0.301898798 |
| | | ID NO: 3385) | | | | | | | |
| 0 | M | K | 0.462242932 | 0.245616902 | 33 | V | L | 0.451975838 | 0.159872004 |
| 711 | E | [stop] | 0.461879161 | 0.191719959 | 266 | D | Y | 0.451699485 | 0.165335876 |
| 357 | K | N | 0.461332764 | 0.184353442 | 497 | E | D | 0.451539434 | 0.154482619 |
| 434 | H | D | 0.461154018 | 0.191223379 | 661 | E | [stop] | 0.45138977 | 0.234896635 |
| 910 | V | E | 0.460870605 | 0.281013173 | 897 | K | N | 0.451376493 | 0.172130787 |
| 922 | E | D | 0.460080408 | 0.286351122 | 894 | S | G | 0.451201568 | 0.216541569 |
| 480 | L | D | 0.459795711 | 0.404684507 | 46 | N | K | 0.450854268 | 0.293319843 |
| 772 | E | G | 0.459510918 | 0.312503946 | 42 | E | [stop] | 0.450047213 | 0.226279727 |
| 369 | A | P | 0.459368992 | 0.154954523 | 20 | K | N | 0.449773662 | 0.196721642 |
| 148 | G | C | 0.459321913 | 0.21989387 | 285 | H | N | 0.44861581 | 0.243329874 |
| 565 | E | [stop] | 0.459284191 | 0.257970072 | 47 | L | V | 0.448453393 | 0.267732388 |
| 472 | K | N | 0.458126194 | 0.217353923 | 953 | D | E | 0.448187279 | 0.183598076 |
| 19 | T | K | 0.458002489 | 0.250652905 | 8 | K | E | 0.447865624 | 0.173510738 |
| 550 | F | L | 0.457885561 | 0.135416611 | 255 | K | N | 0.447654062 | 0.257753112 |
| 642 | E | D | 0.457477443 | 0.18048994 | 965 | Y | [stop] | 0.447638184 | 0.206848878 |
| 761 | F | L | 0.457399802 | 0.126293846 | 381 | L | V | 0.447548148 | 0.24623578 |
| 104 | P | H | 0.457206235 | 0.205670388 | 938 | Q | K | 0.44750144 | 0.297903846 |
| 588 | G | C | 0.457151433 | 0.254991865 | 719 | S | C | 0.4472033 | 0.232249869 |
| 516 | F | L | 0.456927783 | 0.127509134 | 89 | Q | K | 0.447094951 | 0.222907496 |
| 147 | K | N | 0.456444496 | 0.280029247 | 735 | R | L | 0.447058488 | 0.220193339 |
| 651 | P | H | 0.456356549 | 0.186081926 | 673 | E | G | 0.446968171 | 0.213951556 |
| 2 | E | D | 0.456056175 | 0.35763481 | 126 | G | C | 0.446802066 | 0.204738022 |
| 643 | V | G | 0.455368156 | 0.295796806 | 919 | H | D | 0.446668628 | 0.327432207 |
| 524 | K | N | 0.45482233 | 0.143701874 | 23 | G | V | 0.446595867 | 0.2102612 |
| 18 | N | K | 0.454706199 | 0.199478283 | 733 | M | 1 | 0.446594817 | 0.174646778 |
| 5 | R | T | 0.45449471 | 0.277079709 | 490 | R | G | 0.435740618 | 0.182925074 |
| 310 | Q | E | 0.446297431 | 0.123674296 | 789 | E | G | 0.435579914 | 0.162786893 |
| 729 | L | V | 0.445993097 | 0.433135394 | 603 | -- | LE | 0.43556049 | 0.202470667 |
| 455 | W | L | 0.445597501 | 0.281894997 | 442 | R | S | 0.435504028 | 0.210966357 |
| 215 | G | V | 0.445352945 | 0.205217458 | 714 | R | I | 0.435462316 | 0.200883442 |
| 135 | P | T | 0.44528202 | 0.217449002 | 8 | K | R | 0.435212211 | 0.195908908 |
| 936 | R | T | 0.445259832 | 0.32221387 | 854 | N | D | 0.43513717 | 0.067943636 |
| 519 | Q | K | 0.444720886 | 0.28933765 | 335 | E | [stop] | 0.434927464 | 0.21407853 |
| 656 | G | R | 0.444552088 | 0.279063867 | 915 | G | R | 0.434895859 | 0.195491247 |
| 613 | G | R | 0.444378039 | 0.117584873 | 762 | G | C | 0.434868342 | 0.215911162 |
| 16 | D | Y | 0.44433236 | 0.241975919 | 3 | I | T | 0.434607673 | 0.107252687 |
| 5 | R | K | 0.443724261 | 0.262708705 | 406 | E | [stop] | 0.434574625 | 0.271888642 |
| 3 | I | M | 0.443191661 | 0.128675121 | 710 | V | A | 0.434488312 | 0.161462791 |
| 523 | V | L | 0.443126307 | 0.088900743 | 594 | E | Q | 0.434478655 | 0.199232108 |
| 760 | G | C | 0.442544743 | 0.174174731 | 601 | L | M | 0.433295669 | 0.21298138 |
| 27 | P | T | 0.442229152 | 0.271402709 | 194 | --- | DFY | 0.433205 | 0.315807396 |
| 694 | G | D | 0.441607057 | 0.430247861 | 79 | A | S | 0.433187114 | 0.14702693 |
| 695 | E | D | 0.440698297 | 0.174763691 | 913 | NC | FS | 0.432811714 | 0.214195068 |
| 96 | M | I | 0.440309501 | 0.212758418 | 955 | R | S | 0.432632415 | 0.15138175 |
| 234 | G | V | 0.44028737 | 0.19450919 | 793 | ------ | SKTYL (SEQ | 0.432421193 | 0.207758327 |
| | | | | | | | ID NO: 3715) | | |
| 385 | E | D | 0.440128169 | 0.19408182 | 171 | P | H | 0.432364213 | 0.194710101 |
| 744 | Y | H | 0.439198298 | 0.25211241 | 560 | N | S | 0.432346515 | 0.239882019 |
| 519 | Q | H | 0.438343378 | 0.164581049 | 370 | --- | GYK | 0.432297106 | 0.219290605 |
| 385 | E | [stop] | 0.438258279 | 0.212771705 | 321 | P | Q | 0.432271564 | 0.211438092 |
| 793 | S | R | 0.438010456 | 0.160112082 | 979 | LE[stop]GS- | VSSKDLRA | 0.432126183 | 0.250028634 |
| | | | | | | PG (SEQ ID | (SEQ ID NO: | | |
| | | | | | | NO: 3251) | 3820) | | |
| 726 | A | S | 0.437983799 | 0.129329735 | 21 | K | E | 0.431813708 | 0.20570077 |
| 953 | D | Y | 0.437888499 | 0.29124605 | 348 | C | W | 0.431395847 | 0.285738532 |
| 203 | E | [stop] | 0.437866757 | 0.193004717 | 712 | Q | E | 0.430794328 | 0.137430622 |
| 887 | G | V | 0.437831028 | 0.150855683 | 867 | V | A | 0.430546539 | 0.112438125 |
| 189 | G | R | 0.437816984 | 0.195105194 | 902 | H | N | 0.430482041 | 0.210989962 |
| 672 | P | L | 0.437768207 | 0.1420574 | 232 | C | R | 0.430431738 | 0.130635142 |
| 906 | Q | R | 0.437668081 | 0.257388395 | 164 | E | [stop] | 0.43010378 | 0.307258004 |
| 887 | G | R | 0.436446894 | 0.261046568 | 926 | L | V | 0.42049552 | 0.169568285 |
| 6 | I | T | 0.436255483 | 0.311769796 | 873 | S | R | 0.420222785 | 0.189220359 |
| 751 | M | R | 0.436212653 | 0.194544034 | 823 | R | G | 0.420141589 | 0.140425724 |
| 115 | V | A | 0.436134597 | 0.191229151 | 703 | T | A | 0.419927183 | 0.299947391 |
| 348 | C | R | 0.429790014 | 0.254295816 | 265 | K | N | 0.419762272 | 0.205398427 |
| 13 | L | R | 0.429496589 | 0.209797858 | 904 | P | L | 0.419717349 | 0.24717221 |
| 11 | R | W | 0.429311947 | 0.298268587 | 315 | G | A | 0.419275038 | 0.167267502 |
| 944 | Q | E | 0.429084418 | 0.194128082 | 346 | M | I | 0.418933456 | 0.153077303 |
| 974 | K | E | 0.428778767 | 0.120819051 | 301 | V | A | 0.418922077 | 0.253824177 |
| 935 | L | M | 0.428357966 | 0.408223034 | 545 | I | M | 0.418607437 | 0.264461321 |
| 131 | Q | E | 0.427961752 | 0.108783149 | 676 | P | T | 0.41817469 | 0.167866208 |
| 961 | W | R | 0.427770336 | 0.153009954 | 516 | F | S | 0.418152987 | 0.18301751 |
| 508 | F | L | 0.427277307 | 0.150834085 | 790 | G | V | 0.417872524 | 0.17800118 |
| 732 | D | Y | 0.427260152 | 0.232782252 | 890 | G | V | 0.417424955 | 0.242331279 |
| 876 | S | G | 0.427219565 | 0.1654476 | 684 | L | P | 0.41697175 | 0.237298169 |
| 36 | M | I | 0.426965901 | 0.18021585 | 369 | A | T | 0.416965887 | 0.158164268 |
| 699 | E | [stop] | 0.426936027 | 0.247620152 | 890 | G | R | 0.416918523 | 0.30183511 |
| 624 | R | G | 0.426915666 | 0.161800086 | 515 | A | T | 0.416763488 | 0.158965629 |
| 687 | ----- | PTHTL (SEQ | 0.426399688 | 0.235010897 | | | | | |
| | | ID NO: 3626) | | | | | | | |
| 176 | A | G | 0.425859136 | 0.154112817 | 903 | R | G | 0.416689964 | 0.149830948 |
| 256 | K | N | 0.425760398 | 0.195398586 | 898 | K | [stop] | 0.416641263 | 0.154852179 |
| 904 | P | A | 0.425684716 | 0.273763449 | 632 | L | V | 0.416523782 | 0.131108293 |
| 859 | Q | K | 0.425619083 | 0.166409301 | 126 | G | D | 0.41639346 | 0.171080754 |
| 222 | G | [stop] | 0.425285813 | 0.299517445 | 151 | H | R | 0.41621118 | 0.192083944 |
| 20 | K | E | 0.425128158 | 0.147645138 | 480 | L | P | 0.4153828 | 0.153349872 |
| 327 | G | C | 0.425002655 | 0.239317573 | 569 | M | T | 0.415261579 | 0.12705723 |
| 530 | L | P | 0.423859206 | 0.240275284 | 819 | A | S | 0.414776737 | 0.173259385 |
| 175 | E | Q | 0.423850119 | 0.242087732 | 212 | E | [stop] | 0.414560972 | 0.214325617 |
| 797 | L | P | 0.423394833 | 0.254739368 | 104 | P | T | 0.414121539 | 0.241680787 |
| 351 | K | M | 0.423313443 | 0.177944606 | 765 | G | A | 0.413859942 | 0.202334164 |
| 912 | L | M | 0.423204978 | 0.27824291 | 862 | -- | VK | 0.413059952 | 0.195129021 |
| 188 | F | L | 0.422539663 | 0.187750751 | 210 | P | A | 0.412638448 | 0.228860931 |
| 850 | I | M | 0.422459968 | 0.218452121 | 824 | V | A | 0.412207035 | 0.173953175 |
| 391 | K | N | 0.422162984 | 0.158915852 | 736 | N | K | 0.411883437 | 0.18403448 |
| 894 | - | S | 0.42194087 | 0.23660887 | 13 | L | H | 0.411795935 | 0.405614507 |
| 758 | S | R | 0.420859106 | 0.119214586 | 844 | L | V | 0.411372197 | 0.244473235 |
| 941 | K | N | 0.420814047 | 0.266042931 | 973 | W | L | 0.403521777 | 0.16358494 |
| 381 | L | P | 0.42076192 | 0.122089029 | 976 | A | S | 0.403444209 | 0.261893297 |
| 564 | G | C | 0.411344604 | 0.228204596 | 180 | L | P | 0.403389637 | 0.163854455 |
| 694 | G | R | 0.41123482 | 0.211796515 | 220 | A | S | 0.402957864 | 0.279961071 |
| 977 | V | L | 0.411157664 | 0.380351062 | 894 | ------ | SLLKK (SEQ | 0.402797711 | 0.216370575 |
| | | | | | | | ID NO: 3720) | | |
| 142 | E | K | 0.410509302 | 0.15102557 | 739 | R | I | 0.402772732 | 0.234602886 |
| 4 | K | E | 0.410380978 | 0.274892917 | 548 | E | [stop] | 0.402765683 | 0.262561545 |
| 890 | G | D | 0.410337543 | 0.240602631 | 764 | Q | K | 0.402617217 | 0.220740512 |
| 409 | H | D | 0.410132391 | 0.22531365 | 723 | A | D | 0.402461227 | 0.236080429 |
| 563 | S | C | 0.409998896 | 0.206123321 | 934 | F | L | 0.402458138 | 0.384373835 |
| 793 | S | N | 0.409457982 | 0.067541166 | 42 | E | D | 0.401939693 | 0.171540664 |
| 705 | Q | H | 0.409365382 | 0.15278139 | 956 | A | G | 0.401859954 | 0.23877341 |
| 515 | A | D | 0.409252018 | 0.206051204 | 771 | A | D | 0.401428057 | 0.231350403 |
| 382 | S | R | 0.408669778 | 0.157144259 | 15 | K | M | 0.401237871 | 0.256454456 |
| 97 | S | N | 0.408564877 | 0.109922347 | 298 | A | V | 0.401000777 | 0.140487597 |
| 624 | R | I | 0.40845718 | 0.228955853 | 128 | A | P | 0.400992369 | 0.173078759 |
| 568 | P | T | 0.408066084 | 0.284742394 | 511 | Q | H | 0.400978135 | 0.171613013 |
| 702 | R | S | 0.408063786 | 0.129537489 | 26 | G | V | 0.400800405 | 0.212307845 |
| 796 | Y | N | 0.40788333 | 0.311628718 | 591 | ------ | QGREFI (SEQ | 0.400574847 | 0.190655853 |
| | | | | | | | ID NO: 3636) | | |
| 897 | K | R | 0.407876662 | 0.136002906 | 156 | G | S | 0.400389686 | 0.306653761 |
| 292 | A | V | 0.407642755 | 0.163883385 | 728 | N | S | 0.400298817 | 0.177178828 |
| 741 | L | Q | 0.407532982 | 0.11928093 | 917 | ------ | ETHADE | 0.400170477 | 0.15562198 |
| | | | | | | | (SEQ ID NO: | | |
| | | | | | | | 3401) | | |
| 315 | G | C | 0.407147181 | 0.218556644 | 640 | R | G | 0.399931978 | 0.200741 |
| -1 | S | Y | 0.407080752 | 0.324937034 | 254 | I | M | 0.39981124 | 0.209846066 |
| 945 | T | I | 0.407011152 | 0.285905433 | 644 | L | P | 0.399481964 | 0.165702888 |
| 695 | E | [stop] | 0.406081569 | 0.227028835 | 549 | A | S | 0.399416255 | 0.189530269 |
| 956 | A | S | 0.405686952 | 0.185566124 | 528 | L | V | 0.399354304 | 0.147818268 |
| 752 | L | M | 0.405575007 | 0.172103348 | 502 | I | V | 0.399285899 | 0.256373682 |
| 45 | E | [stop] | 0.405531899 | 0.162357698 | 79 | A | D | 0.399080303 | 0.154917165 |
| 487 | G | C | 0.405450681 | 0.290615306 | 753 | I | M | 0.399024046 | 0.268887392 |
| 310 | Q | R | 0.405123752 | 0.12048192 | 588 | G | D | 0.398941525 | 0.112261489 |
| 791 | L | P | 0.404916001 | 0.108993438 | 873 | S | G | 0.392619693 | 0.143564629 |
| 767 | R | I | 0.404746394 | 0.223610078 | 414 | G | D | 0.392615344 | 0.149137614 |
| 538 | G | C | 0.404409405 | 0.233295785 | 237 | A | G | 0.392578525 | 0.167793454 |
| 584 | P | A | 0.403953066 | 0.108926305 | 479 | E | [stop] | 0.392365621 | 0.272905538 |
| 552 | A | D | 0.403929388 | 0.192995621 | 752 | L | V | 0.392234134 | 0.171880044 |
| 648 | N | D | 0.403814843 | 0.290734901 | 692 | R | I | 0.391963575 | 0.221910688 |
| 722 | Y | H | 0.398538883 | 0.164012123 | 683 | s | Y | 0.39187962 | 0.197184801 |
| 550 | - | G | 0.398527591 | 0.353355602 | 568 | P | s | 0.391506615 | 0.094807068 |
| 133 | C | R | 0.398285042 | 0.283233819 | 114 | P | T | 0.391456539 | 0.163794482 |
| 591 | -- | QG | 0.398079043 | 0.133460692 | 341 | V | A | 0.391246425 | 0.087691935 |
| 877 | V | L | 0.398057665 | 0.212468549 | 50 | K | R | 0.39108021 | 0.159163965 |
| 958 | V | A | 0.398007545 | 0.130004197 | 698 | K | R | 0.390885992 | 0.181654156 |
| 903 | R | I | 0.39789959 | 0.321002606 | 979 | L- | V[stop] | 0.3907803 | 0.18994351 |
| 118 | G | D | 0.397657151 | 0.192339782 | 932 | W | G | 0.390757599 | 0.185057669 |
| 745 | A | S | 0.397594938 | 0.285476509 | 519 | Q | R | 0.390675235 | 0.117792262 |
| 914 | C | F | 0.397278541 | 0.29475166 | 140 | K | E | 0.390615529 | 0.123713502 |
| 461 | --- | SFV | 0.39704755 | 0.20205322 | 40 | L | P | 0.390579865 | 0.194510846 |
| 637 | --- | TFE | 0.396824735 | 0.209304074 | 978 | - | [stop] | 0.390537744 | 0.255501032 |
| 855 | R | M | 0.396780958 | 0.191874811 | 509 | S | T | 0.390466368 | 0.117704569 |
| 142 | E | [stop] | 0.396624103 | 0.229993954 | 465 | E | [stop] | 0.390424913 | 0.211758729 |
| 108 | D | N | 0.396298431 | 0.15939576 | 88 | F | S | 0.390363974 | 0.156430305 |
| 730 | ------- | ADDMVRN | 0.395727458 | 0.207712648 | 429 | E | [stop] | 0.390336598 | 0.135919503 |
| | | (SEQ ID NO: | | | | | | | |
| | | 3305) | | | | | | | |
| 241 | T | I | 0.395690613 | 0.131948289 | 783 | --- | TAK | 0.390178711 | 0.143499076 |
| 641 | R | I | 0.395315387 | 0.202249461 | 442 | R | M | 0.390097432 | 0.262199628 |
| 364 | F | L | 0.395209211 | 0.112951976 | 453 | T | A | 0.389911631 | 0.312187594 |
| 739 | R | G | 0.395162717 | 0.191317885 | 923 | Q | H | 0.389855175 | 0.353446475 |
| 446 | A | S | 0.39510798 | 0.254001902 | 666 | V | A | 0.389840585 | 0.169825945 |
| 593 | R | [stop] | 0.395071199 | 0.196636879 | 499 | E | D | 0.38958943 | 0.172940321 |
| 168 | L | P | 0.39502304 | 0.27101743 | 930 | R | G | 0.389517964 | 0.2357312 |
| 890 | G | C | 0.394653545 | 0.224530018 | 847 | ------ | EGQITY | 0.389324278 | 0.122951036 |
| | | | | | | | (SEQ ID NO: | | |
| | | | | | | | 3387) | | |
| 677 | -- | LS | 0.394551417 | 0.187547463 | 846 | V | L | 0.389120343 | 0.259313474 |
| 47 | L | R | 0.394492318 | 0.238759289 | 908 | K | N | 0.38907418 | 0.225076472 |
| 339 | N | S | 0.394482682 | 0.152047471 | 975 | P | T | 0.388901662 | 0.256059318 |
| 316 | R | G | 0.394439897 | 0.159274636 | 783 | T | R | 0.381262501 | 0.118770396 |
| 206 | H | N | 0.394299838 | 0.156799046 | 916 | F | V | 0.380756944 | 0.281228145 |
| 651 | P | A | 0.394024946 | 0.151434436 | 450 | A | T | 0.38074186 | 0.136570467 |
| 441 | R | G | 0.393551449 | 0.150649913 | 906 | Q | E | 0.380700478 | 0.285392821 |
| 325 | L | P | 0.393343386 | 0.140601419 | 29 | K | [stop] | 0.380574061 | 0.171976662 |
| 589 | K | N | 0.3926379 | 0.261890195 | 936 | R | I | 0.38042421 | 0.204558309 |
| 149 | K | N | 0.38882454 | 0.171027465 | 754 | F | I | 0.380277272 | 0.145574058 |
| 691 | L | P | 0.388805401 | 0.14397393 | 315 | G | S | 0.380117687 | 0.143338421 |
| 207 | P | A | 0.387921412 | 0.102883658 | 89 | Q | [stop] | 0.379768129 | 0.102222221 |
| 11 | - | S | 0.387747808 | 0.379461072 | 289 | G | C | 0.379664161 | 0.235845043 |
| 638 | F | L | 0.387272475 | 0.168477543 | 750 | A | T | 0.379378398 | 0.182932261 |
| 558 | V | L | 0.386662896 | 0.254612529 | 216 | G | C | 0.379274317 | 0.176888646 |
| 816 | I | V | 0.386659025 | 0.185203822 | 303 | W | C | 0.379215164 | 0.182222922 |
| 680 | F | L | 0.386638685 | 0.211225716 | 295 | N | K | 0.379144284 | 0.378487654 |
| 329 | P | T | 0.386489681 | 0.220048383 | 919 | H | Y | 0.379137691 | 0.321018649 |
| 576 | D | G | 0.386151413 | 0.113653327 | 726 | A | D | 0.379067543 | 0.145080733 |
| 225 | G | V | 0.386137184 | 0.239109613 | 133 | C | S | 0.378841599 | 0.162936296 |
| 22 | A | G | 0.385839168 | 0.336984972 | 497 | E | [stop] | 0.378292682 | 0.202801468 |
| 146 | D | E | 0.385277721 | 0.095712474 | 444 | E | K | 0.378042967 | 0.318660643 |
| 507 | G | R | 0.385233777 | 0.212044464 | 693 | I | M | 0.378036899 | 0.225823359 |
| 523 | V | I | 0.385109283 | 0.152511446 | 587 | F | L | 0.377947216 | 0.117981043 |
| 501 | S | G | 0.385073546 | 0.140125388 | 291 | E | D | 0.377733323 | 0.142365006 |
| 763 | R | L | 0.38502172 | 0.191531655 | 85 | W | S | 0.377648166 | 0.097279693 |
| 705 | Q | E | 0.384851421 | 0.17568818 | 165 | R | M | 0.377647305 | 0.161201002 |
| 82 | H | D | 0.383907018 | 0.103874584 | 569 | M | I | 0.377387614 | 0.195898876 |
| 794 | K | N | 0.383803253 | 0.195192527 | 247 | I | T | 0.37729282 | 0.165305688 |
| 979 | LE[stop]GSPG | VSSKDLR | 0.38375861 | 0.240184851 | 513 | - | N | 0.377106209 | 0.14731404 |
| | (SEQ ID NO: | (SEQ ID NO: | | | | | | | |
| | 3251) | 3819) | | | | | | | |
| 894 | S | R | 0.383344078 | 0.273603195 | 754 | F | L | 0.376911731 | 0.164266559 |
| 639 | E | [stop] | 0.383174826 | 0.193125393 | 21 | K | [stop] | 0.376868031 | 0.199468055 |
| 655 | I | M | 0.383102617 | 0.208514699 | 268 | A | T | 0.376839819 | 0.129211081 |
| 261 | L | V | 0.382856978 | 0.19611714 | 672 | P | T | 0.376830532 | 0.204970386 |
| 480 | L | R | 0.382841683 | 0.252187108 | 735 | R | [stop] | 0.376814295 | 0.09621637 |
| 489 | L | V | 0.38262991 | 0.16124555 | 147 | K | E | 0.376789616 | 0.140417542 |
| 134 | Q | E | 0.382580711 | 0.180510987 | 904 | P | R | 0.37666328 | 0.185106225 |
| 650 | -- | PA | 0.382487274 | 0.372015728 | 712 | Q | H | 0.376030218 | 0.227827888 |
| 630 | P | H | 0.381699363 | 0.211396524 | 92 | P | T | 0.368981275 | 0.236532466 |
| 21 | K | R | 0.381603442 | 0.1634713 | 292 | A | T | 0.36879806 | 0.193425471 |
| 677 | --- | LSR | 0.381372384 | 0.163400905 | 465 | E | D | 0.368752489 | 0.224455423 |
| 284 | P | T | 0.381276843 | 0.171865261 | 189 | -------- | GQRALDFY | 0.368745456 | 0.227136846 |
| | | | | | | | (SEQ ID NO: | | |
| | | | | | | | 3448) | | |
| 2 | E | V | 0.375325693 | 0.197955097 | 805 | T | A | 0.368671629 | 0.11272788 |
| 184 | S | I | 0.375300851 | 0.252137747 | 947 | K | E | 0.368551642 | 0.227968732 |
| 163 | H | D | 0.3751698 | 0.208290707 | 148 | G | D | 0.36788165 | 0.139635081 |
| 677 | L | P | 0.375131489 | 0.090158552 | 129 | C | W | 0.367758112 | 0.199915902 |
| 44 | L | P | 0.374906966 | 0.249472829 | 129 | C | [stop] | 0.367708546 | 0.192643557 |
| 606 | G | V | 0.374739683 | 0.285964981 | 98 | R | T | 0.367673403 | 0.174398036 |
| 937 | S | G | 0.374669762 | 0.248499289 | 478 | C | W | 0.367598979 | 0.111931907 |
| 727 | K | N | 0.374273348 | 0.164838535 | 228 | L | M | 0.367328433 | 0.24869867 |
| 734 | V | A | 0.374244799 | 0.121134147 | 547 | P | H | 0.367324308 | 0.220855574 |
| 902 | H | Q | 0.374087073 | 0.175219897 | 105 | K | N | 0.367245695 | 0.155463083 |
| 398 | F | L | 0.373909011 | 0.239653674 | 597 | W | R | 0.367058721 | 0.142955463 |
| 845 | K | N | 0.373742099 | 0.158752661 | 328 | F | L | 0.366955458 | 0.100787228 |
| 822 | D | N | 0.373424135 | 0.138952336 | 469 | E | [stop] | 0.366917206 | 0.180496612 |
| 136 | L | M | 0.372880562 | 0.202180857 | 130 | S | T | 0.366622403 | 0.127263853 |
| 543 | K | E | 0.372880222 | 0.146877967 | 283 | Q | E | 0.366530641 | 0.247989672 |
| 244 | Q | H | 0.372873077 | 0.184616643 | 958 | V | L | 0.366470474 | 0.270699212 |
| 403 | L | R | 0.372697479 | 0.330913239 | 673 | E | Q | 0.366346139 | 0.219545941 |
| 679 | R | I | 0.372176403 | 0.370324076 | 118 | G | C | 0.366255984 | 0.265748809 |
| 738 | A | D | 0.372074442 | 0.291834989 | 848 | G | V | 0.366195099 | 0.200861406 |
| 155 | F | L | 0.371845015 | 0.114679195 | 923 | Q | L | 0.366184575 | 0.233234243 |
| 174 | P | R | 0.371603352 | 0.137168151 | 357 | K | R | 0.366148171 | 0.185792239 |
| 919 | H | N | 0.371556993 | 0.327290993 | 623 | ------ | RRTRQD | 0.365486053 | 0.26101804 |
| | | | | | | | (SEQ ID NO: | | |
| | | | | | | | 3683) | | |
| 944 | Q | H | 0.37144256 | 0.338788753 | 85 | W | C | 0.365346783 | 0.146084706 |
| 164 | E | G | 0.370935537 | 0.216755032 | 376 | ----- | ALLPY (SEQ | 0.365321474 | 0.191317647 |
| | | | | | | | ID NO: 3319) | | |
| 197 | S | G | 0.370856052 | 0.178568608 | 356 | E | D | 0.365050343 | 0.136074432 |
| 840 | N | K | 0.370814634 | 0.142530771 | 262 | A | S | 0.365012551 | 0.204615446 |
| 13 | L | M | 0.370495333 | 0.29466367 | 774 | Q | K | 0.359747336 | 0.182131652 |
| 488 | D | N | 0.370055302 | 0.226946737 | 439 | E | D | 0.359587685 | 0.134619305 |
| 929 | A | P | 0.370027168 | 0.168555798 | 198 | I | T | 0.359370526 | 0.173615874 |
| 580 | L | V | 0.36995513 | 0.139984948 | 156 | G | C | 0.359055571 | 0.173590319 |
| 135 | P | A | 0.369933138 | 0.10604161 | 399 | G | C | 0.358922413 | 0.255017848 |
| 342 | D | Y | 0.369924443 | 0.189241086 | 59 | S | T | 0.358703019 | 0.109042363 |
| 959 | ET | AV | 0.369879201 | 0.114167508 | 93 | V | M | 0.358615623 | 0.161948363 |
| 557 | T | A | 0.369640872 | 0.087836911 | 674 | G | [stop] | 0.358503233 | 0.220631194 |
| 6 | I | V | 0.369460173 | 0.192497769 | 539 | K | N | 0.358074633 | 0.087009621 |
| 765 | G | S | 0.3649426 | 0.100657536 | 709 | E | D | 0.357944736 | 0.136689683 |
| 717 | ---- | GYSR (SEQ | 0.364903794 | 0.186125273 | 120 | E | G | 0.357933511 | 0.168382586 |
| | | ID NO: 3457) | | | | | | | |
| 199 | H | Y | 0.364586783 | 0.168211628 | 494 | F | L | 0.357874746 | 0.139367085 |
| 796 | Y | H | 0.364521403 | 0.145575579 | 272 | G | V | 0.357428523 | 0.207170798 |
| 237 | A | P | 0.364453395 | 0.150681341 | 527 | N | I | 0.357320226 | 0.086164887 |
| 768 | T | A | 0.36435574 | 0.18512185 | 236 | V | A | 0.357249373 | 0.125737046 |
| 513 | N | D | 0.364305814 | 0.16260499 | 974 | K | N | 0.357242055 | 0.190403244 |
| 823 | RV | LS | 0.364237044 | 0.11377221 | 10 | RR | PG | 0.356712463 | 0.324298272 |
| 656 | G | A | 0.364010939 | 0.135958583 | 39 | D | Y | 0.356585187 | 0.235756832 |
| 276 | P | T | 0.363878534 | 0.201304545 | 579 | N | S | 0.3558347 | 0.181516226 |
| 214 | I | V | 0.363876419 | 0.142178855 | 214 | I | M | 0.355779849 | 0.142887254 |
| 300 | I | V | 0.363823907 | 0.234997169 | 843 | E | [stop] | 0.355689249 | 0.225441771 |
| 769 | F | S | 0.363687361 | 0.079831237 | 526 | ---- | LNLY (SEQ | 0.355597159 | 0.179351732 |
| | | | | | | | ID NO: 3563) | | |
| 182 | T | R | 0.363686071 | 0.201742372 | 667 | I | M | 0.355548811 | 0.239632986 |
| 677 | L | V | 0.363578004 | 0.138045802 | 559 | I | V | 0.355478406 | 0.171281999 |
| 796 | Y | C | 0.363566923 | 0.281557418 | 706 | A | S | 0.355431605 | 0.116949175 |
| 5 | R | S | 0.363258223 | 0.211185531 | 11 | RR | TS | 0.35536352 | 0.272262643 |
| 298 | A | S | 0.36320777 | 0.211187305 | 865 | L | Q | 0.355287262 | 0.164676142 |
| 594 | E | [stop] | 0.36278807 | 0.205352129 | 946 | N | K | 0.355277474 | 0.180093688 |
| 105 | K | R | 0.362205009 | 0.140104618 | 689 | HI | PV | 0.355052108 | 0.144577201 |
| 907 | E | Q | 0.362024887 | 0.226228418 | 898 | K | N | 0.354894826 | 0.200062158 |
| 509 | S | G | 0.361807445 | 0.13953396 | 950 | -- | GN | 0.354845909 | 0.167057981 |
| 110 | R | I | 0.361752083 | 0.138681372 | 332 | P | T | 0.354796362 | 0.20270742 |
| 406 | E | Q | 0.361750488 | 0.303638253 | 323 | Q | E | 0.354759964 | 0.249399571 |
| 470 | A | V | 0.361349462 | 0.10686226 | 42 | E | A | 0.354721226 | 0.213005644 |
| 4 | K | [stop] | 0.36129388 | 0.179352157 | 644 | L | V | 0.351676716 | 0.163471035 |
| 362 | K | E | 0.361196668 | 0.232368389 | 78 | K | E | 0.35167205 | 0.128519193 |
| 713 | R | G | 0.3607467 | 0.181817788 | 272 | G | C | 0.351365895 | 0.208785029 |
| 857 | K | N | 0.360715256 | 0.172046815 | 157 | -------- | RCNVSEHE | 0.351115058 | 0.126463217 |
| | | | | | | | (SEQ ID NO: | | |
| | | | | | | | 3661) | | |
| 120 | E | D | 0.36030686 | 0.214810208 | 883 | S | R | 0.351093302 | 0.143213807 |
| 277 | K | E | 0.36002957 | 0.210892547 | 917 | E | V | 0.350763439 | 0.206641731 |
| 477 | RCELK (SEQ | SFSSH (SEQ | 0.360015336 | 0.177473578 | 843 | E | D | 0.350569244 | 0.142523946 |
| | ID NO: 3285) | ID NO: 3696) | | | | | | | |
| 532 | I | T | 0.359759307 | 0.145072322 | 870 | D | Y | 0.350431061 | 0.194706521 |
| 22 | A | T | 0.354629728 | 0.083320918 | 393 | F | V | 0.35027948 | 0.168738586 |
| 948 | T | S | 0.354488334 | 0.198422577 | 162 | E | K | 0.350236681 | 0.12523983 |
| 16 | D | E | 0.354450775 | 0.187189495 | 119 | N | D | 0.350147467 | 0.235898677 |
| 170 | S | Y | 0.354344814 | 0.160709939 | 306 | L | M | 0.349889759 | 0.165537841 |
| 862 | | VKDLS (SEQ | 0.354059938 | 0.179170942 | 110 | R | T | 0.349523294 | 0.289863999 |
| | | ID NO: 3781) | | | | | | | |
| 249 | E | [stop] | 0.354016591 | 0.294486267 | 976 | A | D | 0.34941868 | 0.241042383 |
| 531 | I | M | 0.353941253 | 0.095481374 | 914 | C | W | 0.349231308 | 0.169568161 |
| 266 | D | H | 0.35392753 | 0.237329699 | 115 | V | M | 0.349160578 | 0.17839763 |
| 859 | Q | E | 0.353923377 | 0.126451964 | 863 | K | N | 0.348978081 | 0.175915912 |
| 113 | I | V | 0.353631334 | 0.187941798 | 830 | K | R | 0.348789882 | 0.11782242 |
| 136 | L | P | 0.353572714 | 0.240617705 | 564 | G | S | 0.348654331 | 0.240781896 |
| 503 | L | M | 0.353400839 | 0.174768283 | 647 | S | I | 0.348570495 | 0.163208612 |
| 51 | P | R | 0.353321532 | 0.126698252 | 617 | E | D | 0.348384104 | 0.103608149 |
| 179 | E | D | 0.353270131 | 0.108592116 | 262 | A | T | 0.348231917 | 0.222328473 |
| 31 | L | V | 0.353260601 | 0.168619621 | 713 | R | I | 0.348163293 | 0.202182526 |
| 502 | I | F | 0.353258477 | 0.139633145 | 893 | L | P | 0.348133135 | 0.24849422 |
| 378 | L | M | 0.353221613 | 0.189998728 | 202 | R | G | 0.347997162 | 0.177282082 |
| 890 | G | A | 0.353138339 | 0.149947604 | 806 | S | Y | 0.347673828 | 0.200543155 |
| 913 | N | K | 0.353092797 | 0.294888192 | 391 | K | R | 0.347608788 | 0.122435715 |
| 956 | A | D | 0.352997131 | 0.204713576 | 683 | S | C | 0.34755615 | 0.102168244 |
| 158 | C | W | 0.352758393 | 0.130405614 | 446 | A | T | 0.347296208 | 0.236243043 |
| 157 | ---- | RCNV (SEQ | 0.352566351 | 0.116984328 | 282 | P | A | 0.347073665 | 0.253113968 |
| | | ID NO: 3658) | | | | | | | |
| 771 | A | G | 0.352390901 | 0.141133059 | 580 | L | P | 0.347062657 | 0.078573865 |
| 227 | A | G | 0.352335693 | 0.141777326 | 895 | L | P | 0.347059979 | 0.152424473 |
| 202 | RE | G- | 0.352321171 | 0.210660545 | 929 | A | T | 0.34702013 | 0.306789031 |
| 99 | V | F | 0.352314021 | 0.162936095 | 555 | F | L | 0.343270194 | 0.098281937 |
| 643 | V | E | 0.352268894 | 0.209333581 | 294 | N | D | 0.343264324 | 0.126839815 |
| 41 | R | I | 0.352205261 | 0.321737078 | 553 | N | D | 0.342736197 | 0.153294035 |
| 387 | R | P | 0.352184692 | 0.159814147 | 893 | L | M | 0.342736077 | 0.179172833 |
| 539 | K | E | 0.351957196 | 0.146275596 | 951 | N | K | 0.342592943 | 0.278844401 |
| 478 | C | F | 0.351788403 | 0.313141443 | 51 | P | T | 0.342576973 | 0.1929364 |
| 942 | K | E | 0.351775756 | 0.256493816 | 649 | I | T | 0.342534817 | 0.270208479 |
| 36 | M | I | 0.351715805 | 0.097577134 | 175 | E | D | 0.342455704 | 0.202360388 |
| 108 | D | Y | 0.347014656 | 0.291577591 | 823 | R | S | 0.341965728 | 0.273152096 |
| 258 | E | [stop] | 0.34694757 | 0.281979872 | 219 | C | R | 0.341954249 | 0.136482174 |
| 673 | E | A | 0.346691172 | 0.265253287 | 283 | Q | R | 0.341949927 | 0.224313066 |
| 950 | G | D | 0.346646349 | 0.128298199 | 444 | E | [stop] | 0.341881438 | 0.217688103 |
| 792 | P | T | 0.346487957 | 0.236073016 | 649 | I | V | 0.341655494 | 0.148589673 |
| 673 | E | [stop] | 0.346388527 | 0.198074161 | 854 | N | K | 0.341614877 | 0.157948422 |
| 150 | P | R | 0.34632855 | 0.278480507 | 514 | C | S | 0.34160113 | 0.231141571 |
| 456 | L | P | 0.345951509 | 0.161500864 | 623 | ---- | RRTR (SEQ | 0.341527608 | 0.187073234 |
| | | | | | | | ID NO: 3681) | | |
| 790 | G | R | 0.345911786 | 0.179210019 | 585 | L | M | 0.341496703 | 0.21431877 |
| 647 | S | T | 0.345819661 | 0.158521168 | 211 | -- | LE | 0.341207432 | 0.169230112 |
| 542 | F | S | 0.345619595 | 0.191970857 | 544 | K | E | 0.341142267 | 0.208342511 |
| 841 | G | D | 0.345447865 | 0.129392183 | 478 | C | R | 0.341091687 | 0.148433288 |
| 57 | P | A | 0.345371652 | 0.147875225 | 858 | R | G | 0.340977066 | 0.206052559 |
| 578 | P | R | 0.345346371 | 0.12075926 | 172 | H | D | 0.340873936 | 0.298188428 |
| 793 | S | I | 0.345235059 | 0.262377638 | 16 | D | A | 0.340771918 | 0.308121625 |
| 453 | T | S | 0.345118763 | 0.097101409 | 525 | K | N | 0.340626838 | 0.147516442 |
| 651 | P | R | 0.345088622 | 0.208316961 | 532 | I | V | 0.340576058 | 0.099088927 |
| 556 | Y | [stop] | 0.345070339 | 0.114662396 | 520 | K | [stop] | 0.34056167 | 0.228510512 |
| 86 | E | [stop] | 0.344943839 | 0.21976554 | 743 | Y | [stop] | 0.340397436 | 0.102396798 |
| 646 | S | G | 0.344888595 | 0.154435246 | 344 | W | C | 0.340364668 | 0.176812201 |
| 592 | G | C | 0.34478874 | 0.240350052 | 220 | A | G | 0.340276978 | 0.133945921 |
| 49 | K | N | 0.344659946 | 0.130706516 | 186 | G | V | 0.340265085 | 0.116877863 |
| 586 | A | D | 0.344294219 | 0.15117877 | 694 | G | C | 0.340225482 | 0.309935909 |
| 166 | L | V | 0.34415435 | 0.139737754 | 411 | E | Q | 0.340144727 | 0.282548314 |
| 726 | A | P | 0.344144415 | 0.164178243 | 406 | E | G | 0.340120492 | 0.140875629 |
| 666 | V | L | 0.344130904 | 0.155760915 | 573 | F | L | 0.340030507 | 0.166015227 |
| 749 | D | H | 0.344052929 | 0.242192495 | 52 | E | [stop] | 0.336207682 | 0.211986135 |
| 486 | Y | C | 0.34395063 | 0.130965705 | 299 | Q | E | 0.336024324 | 0.156699489 |
| 134 | Q | K | 0.343594633 | 0.210709609 | 183 | YS | WM | 0.335855997 | 0.179538112 |
| 91 | D | H | 0.34352508 | 0.153686099 | 194 | D | Y | 0.335755348 | 0.131644969 |
| 40 | LR | PV | 0.343506493 | 0.155292328 | 213 | Q | R | 0.335726769 | 0.209853061 |
| 12 | R | T | 0.343490891 | 0.187270573 | 802 | A | D | 0.33571172 | 0.168573673 |
| 653 | N | D | 0.343487264 | 0.148663517 | 163 | H | N | 0.33571123 | 0.197315666 |
| 52 | E | Q | 0.343438912 | 0.247941408 | 943 | Y | C | 0.335604909 | 0.172843558 |
| 8 | K | Q | 0.343298615 | 0.279455517 | 118 | G | S | 0.335544316 | 0.125891126 |
| 458 | A | G | 0.339794018 | 0.171435317 | 758 | S | G | 0.335513561 | 0.149050456 |
| 675 | C | [stop] | 0.339687357 | 0.208292109 | 941 | K | [stop] | 0.335374859 | 0.192348189 |
| 576 | D | Y | 0.339621402 | 0.21774439 | 279 | ------- | TLPPQPH | 0.335305655 | 0.144688363 |
| | | | | | | | (SEQ ID NO: | | |
| | | | | | | | 3755) | | |
| 787 | A | S | 0.339526186 | 0.318305548 | 632 | LF | PV | 0.335263893 | 0.113883053 |
| 537 | G | C | 0.339454064 | 0.174110887 | 894 | ------ | SLLKKR | 0.335263893 | 0.141289409 |
| | | | | | | | (SEQ ID NO: | | |
| | | | | | | | 3721) | | |
| 185 | -- | LG | 0.339451721 | 0.186103153 | 943 | Y | [stop] | 0.335115123 | 0.291608446 |
| 844 | L | P | 0.339318044 | 0.191881119 | 38 | P | R | 0.33481965 | 0.113021039 |
| 712 | Q | K | 0.339288003 | 0.193891353 | 616 | I | F | 0.334790976 | 0.107803908 |
| 591 | Q | R | 0.339223049 | 0.160616368 | 134 | Q | H | 0.334549336 | 0.158461695 |
| 169 | L | P | 0.339210958 | 0.127439702 | 186 | G | C | 0.334321874 | 0.156717674 |
| 923 | ----- | QAALN (SEQ | 0.339143383 | 0.169170821 | 184 | S | G | 0.334296555 | 0.223929833 |
| | | ID NO: 3631) | | | | | | | |
| 623 | R | S | 0.339131953 | 0.245088648 | 765 | G | C | 0.33423513 | 0.213904011 |
| 589 | K | Q | 0.33901987 | 0.177422866 | 687 | P | T | 0.334191461 | 0.22545553 |
| 522 | G | V | 0.338985606 | 0.226282565 | 803 | --- | QYT | 0.33418367 | 0.096860089 |
| 204 | S | T | 0.338673547 | 0.170845305 | 374 | Q | R | 0.334175524 | 0.104826318 |
| 698 | K | E | 0.338580473 | 0.129708045 | 455 | W | C | 0.334165051 | 0.186741008 |
| 497 | E | V | 0.338306724 | 0.13489235 | 552 | ----- | ANRFY (SEQ | 0.333923423 | 0.258649392 |
| | | | | | | | ID NO: 3327) | | |
| 23 | G | S | 0.338162596 | 0.15304761 | 407 | K | R | 0.333913165 | 0.142719617 |
| 29 | K | R | 0.337989172 | 0.147861886 | 175 | E | K | 0.333834455 | 0.196225639 |
| 716 | G | V | 0.337974681 | 0.202399788 | 610 | ----- | LANGR (SEQ | 0.333428825 | 0.102899397 |
| | | | | | | | ID NO: 3536) | | |
| 703 | T | S | 0.337889214 | 0.141977828 | 127 | F | I | 0.329561201 | 0.268089932 |
| 979 | LE[stop]GSPG | VSSKDLE | 0.337814175 | 0.168342402 | 837 | T | S | 0.329510402 | 0.099725089 |
| | (SEQ ID NO: | (SEQ ID NO: | | | | | | | |
| | 3251) | 3805) | | | | | | | |
| 240 | L | M | 0.3377179 | 0.151631422 | 704 | I | T | 0.329114566 | 0.113551049 |
| 950 | G | C | 0.337265205 | 0.234973706 | 387 | R | L | 0.328928103 | 0.199189713 |
| 7 | N | S | 0.337036852 | 0.185037778 | 171 | P | R | 0.328685191 | 0.279786527 |
| 64 | A | P | 0.336967696 | 0.255179815 | 767 | R | T | 0.328611454 | 0.173820273 |
| 795 | T | S | 0.336837648 | 0.117371137 | 597 | W | L | 0.328585458 | 0.282536549 |
| 480 | L | Q | 0.336803159 | 0.213915334 | 955 | R | G | 0.328533511 | 0.252801289 |
| 600 | L | V | 0.336801383 | 0.230766925 | 629 | E | [stop] | 0.328472442 | 0.226070443 |
| 175 | E | [stop] | 0.336712437 | 0.187755487 | 699 | E | G | 0.328340286 | 0.161755276 |
| 63 | R | S | 0.336640982 | 0.183725757 | 564 | G | A | 0.328244232 | 0.11512512 |
| 394 | A | P | 0.336388779 | 0.125201204 | 129 | C | F | 0.327975914 | 0.184885596 |
| 230 | ---- | DACM (SEQ | 0.333428825 | 0.108521075 | 26 | G | S | 0.327861024 | 0.174859434 |
| | | ID NO: 3341) | | | | | | | |
| 848 | G | S | 0.333406808 | 0.165245749 | 199 | H | N | 0.327823226 | 0.25447122 |
| 630 | P | R | 0.333389309 | 0.182782946 | 701 | Q | R | 0.327746296 | 0.151982714 |
| 442 | R | G | 0.333281333 | 0.186150848 | 186 | G | D | 0.327613843 | 0.101552272 |
| 836 | M | T | 0.33320739 | 0.215623837 | 422 | E | D | 0.327579534 | 0.227939955 |
| 222 | G | V | 0.333139545 | 0.173506426 | 924 | A | T | 0.327501843 | 0.29494568 |
| 21 | K | T | 0.333022379 | 0.190202016 | 176 | A | P | 0.32741005 | 0.239900376 |
| 696 | S | I | 0.332955668 | 0.138037632 | 499 | E | K | 0.327284744 | 0.159757942 |
| 635 | A | T | 0.332902532 | 0.130552446 | 546 | K | R | 0.327156617 | 0.166513946 |
| 551 | E | G | 0.332833114 | 0.158314375 | 556 | Y | H | 0.327151432 | 0.118520339 |
| 780 | D | Y | 0.332787267 | 0.203141483 | 548 | --- | EAF | 0.326965289 | 0.171181066 |
| 47 | L | M | 0.332771785 | 0.228474741 | 901 | S | I | 0.326880206 | 0.320148616 |
| 347 | V | L | 0.332766547 | 0.164853137 | 14 | V | I | 0.326870011 | 0.276842054 |
| 841 | G | C | 0.332584425 | 0.2483922 | 814 | F | L | 0.32685269 | 0.084563864 |
| 593 | R | I | 0.332546881 | 0.22140312 | 157 | ------ | RCNVSE | 0.326801479 | 0.200654893 |
| | | | | | | | (SEQ ID NO: | | |
| | | | | | | | 3660) | | |
| 749 | D | Y | 0.332359902 | 0.199451757 | 250 | H | R | 0.326584294 | 0.078102923 |
| 27 | P | S | 0.332358372 | 0.306966339 | 730 | A | V | 0.326443401 | 0.110931779 |
| 276 | P | H | 0.332221583 | 0.26420075 | 497 | E | Q | 0.326193187 | 0.212891542 |
| 293 | Y | [stop] | 0.332046234 | 0.133526657 | 536 | K | R | 0.326129704 | 0.20597101 |
| 3 | I | N | 0.332004357 | 0.072687293 | 906 | Q | P | 0.326073598 | 0.193779388 |
| 642 | ---- | EVLD (SEQ | 0.331972419 | 0.22538863 | 243 | Y | D | 0.326001836 | 0.130392708 |
| | | ID NO: 3404) | | | | | | | |
| 620 | L | P | 0.331807594 | 0.15763111 | 786 | L | Q | 0.32241581 | 0.22201146 |
| 456 | L | V | 0.331754102 | 0.143226803 | 4 | K | M | 0.32231147 | 0.124043743 |
| 130 | S | G | 0.331571239 | 0.167684126 | 781 | W | R | 0.322196176 | 0.263818038 |
| 629 | E | K | 0.33154282 | 0.153428302 | 182 | T | I | 0.322044203 | 0.109310181 |
| 950 | G | V | 0.331464709 | 0.229681218 | 888 | R | G | 0.322001059 | 0.172130189 |
| 328 | F | Y | 0.331454046 | 0.090600532 | 388 | K | N | 0.321769292 | 0.13958088 |
| 303 | W | S | 0.331070804 | 0.245928403 | 504 | D | Y | 0.321517406 | 0.182186572 |
| 421 | W | C | 0.330779828 | 0.216037825 | 260 | R | I | 0.321461619 | 0.146534668 |
| 351 | K | R | 0.330630005 | 0.142537112 | 695 | E | Q | 0.321451268 | 0.199405121 |
| 498 | A | T | 0.33049042 | 0.166213318 | 960 | T | A | 0.321351275 | 0.243570837 |
| 937 | S | T | 0.330380882 | 0.231058955 | 496 | I | F | 0.321275456 | 0.162860461 |
| 592 | OR | DN | 0.329593548 | 0.300041765 | 454 | D | H | 0.321034191 | 0.123925099 |
| 798 | S | F | 0.325769587 | 0.320454472 | 859 | Q | H | 0.321009248 | 0.15665955 |
| 882 | S | G | 0.325732755 | 0.141569252 | 432 | S | I | 0.32093586 | 0.219919612 |
| 759 | R | G | 0.325319087 | 0.080028833 | 120 | E | Q | 0.320905282 | 0.134126668 |
| 576 | D | V | 0.325192282 | 0.239519469 | 359 | E | [stop] | 0.320840565 | 0.172779106 |
| 309 | W | [stop] | 0.325098891 | 0.096106342 | 474 | E | [stop] | 0.320753733 | 0.198938474 |
| 554 | R | I | 0.325075441 | 0.185726803 | 609 | K | R | 0.320654761 | 0.097190768 |
| 483 | Q | H | 0.324598695 | 0.153049426 | 654 | L | P | 0.320340402 | 0.21351518 |
| 979 | E | VSSKDQ | 0.324398559 | 0.118712651 | 344 | W | G | 0.32013599 | 0.133467654 |
| | | (SEQ ID NO: | | | | | | | |
| | | 3823) | | | | | | | |
| 834 | G | C | 0.324348652 | 0.175539945 | 629 | E | D | 0.319764058 | 0.097801219 |
| 719 | S | Y | 0.324298439 | 0.22105488 | 631 | A | D | 0.319695703 | 0.120854121 |
| 842 | K | R | 0.324267597 | 0.102772814 | 124 | S | Y | 0.319588026 | 0.148095027 |
| 97 | S | T | 0.324252325 | 0.240123255 | 244 | Q | R | 0.319581236 | 0.174412151 |
| 172 | H | N | 0.324047776 | 0.168532939 | 338 | A | D | 0.319500211 | 0.171228389 |
| 692 | R | G | 0.324024313 | 0.134914995 | 634 | V | L | 0.3194918 | 0.113193905 |
| 39 | D | V | 0.324012084 | 0.186802864 | 91 | D | N | 0.319468455 | 0.231799127 |
| 776 | T | I | 0.323918216 | 0.153171775 | 740 | D | E | 0.319448668 | 0.093677265 |
| 652 | M | T | 0.323898442 | 0.13705991 | 942 | K | R | 0.319440348 | 0.184998826 |
| 611 | A | V | 0.323836429 | 0.18975125 | 146 | D | Y | 0.319268754 | 0.209601725 |
| 658 | D | G | 0.323834837 | 0.116577804 | 513 | N | K | 0.319264079 | 0.180017602 |
| 158 | C | [stop] | 0.323773158 | 0.093674966 | 366 | Q | H | 0.318971922 | 0.184226775 |
| 887 | G | A | 0.32369757 | 0.19151617 | 477 | R | G | 0.318963003 | 0.179227033 |
| 337 | Q | H | 0.323607141 | 0.165283008 | 947 | K | R | 0.318930494 | 0.25585521 |
| 319 | A | D | 0.323458799 | 0.152084781 | 478 | C | S | 0.318576968 | 0.151506435 |
| 215 | | GGNSCA | 0.323334457 | 0.165215546 | 94 | G | A | 0.315344942 | 0.125574217 |
| | | (SEQ ID NO: | | | | | | | |
| | | 3431) | | | | | | | |
| 351 | K | N | 0.323273003 | 0.138737748 | 509 | S | R | 0.315237336 | 0.198196247 |
| 878 | - | I | 0.323133111 | 0.265099492 | 715 | A | S | 0.314795788 | 0.184022977 |
| 597 | W | C | 0.323039345 | 0.210227048 | 639 | E | G | 0.314490675 | 0.131536259 |
| 85 | W | G | 0.3230112 | 0.140970302 | 485 | W | R | 0.314444162 | 0.077460473 |
| 830 | K | E | 0.322976082 | 0.171606667 | 529 | Y | [stop] | 0.314338149 | 0.096977512 |
| 193 | -- | LD | 0.322600674 | 0.167338288 | 773 | R | M | 0.314128132 | 0.191934874 |
| 350 | V | A | 0.32248331 | 0.252994511 | 227 | A | D | 0.313893012 | 0.086820124 |
| 443 | S | G | 0.318453544 | 0.181417518 | 865 | L | V | 0.313870986 | 0.093939035 |
| 766 | K | E | 0.318255467 | 0.119279294 | 25 | T | S | 0.313828907 | 0.165926738 |
| 557 | T | S | 0.318254881 | 0.136960287 | 206 | H | R | 0.313540953 | 0.153060153 |
| 39 | D | E | 0.318241109 | 0.177504749 | 33 | V | I | 0.313378588 | 0.092743144 |
| 586 | A | S | 0.318046156 | 0.197164692 | 736 | N | S | 0.313292021 | 0.139875641 |
| 270 | A | P | 0.317952258 | 0.133471459 | 613 | G | A | 0.313219371 | 0.139952239 |
| 707 | A | S | 0.317797903 | 0.176472631 | 472 | K | R | 0.313201874 | 0.163543589 |
| 173 | K | N | 0.317699885 | 0.158843579 | 149 | --- | KPH | 0.313073613 | 0.111009375 |
| 676 | P | R | 0.317616441 | 0.273323665 | 966 | R | I | 0.313069041 | 0.220268045 |
| 409 | H | N | 0.31739526 | 0.238962249 | 847 | E | [stop] | 0.312986862 | 0.248850102 |
| 878 | N | D | 0.317341485 | 0.123856244 | 892 | A | V | 0.312917635 | 0.236911004 |
| 967 | K | E | 0.317328223 | 0.198885809 | 322 | L | P | 0.312907638 | 0.167614176 |
| 405 | L | M | 0.317316848 | 0.232382071 | 947 | K | N | 0.312809501 | 0.23804854 |
| 759 | R | T | 0.317284234 | 0.210047842 | 820 | D | Y | 0.312669916 | 0.196444965 |
| 505 | I | M | 0.317274558 | 0.129635964 | 627 | Q | E | 0.312477809 | 0.180929549 |
| 612 | N | D | 0.317252502 | 0.181380961 | 20 | K | T | 0.312450252 | 0.306509245 |
| 862 | V | A | 0.317158438 | 0.090072044 | 914 | C | G | 0.312434698 | 0.246328459 |
| 295 | -N | LS | 0.317076665 | 0.155046903 | 793 | S | G | 0.312385644 | 0.182436917 |
| 165 | R | G | 0.317047785 | 0.17842685 | 411 | E | D | 0.312132984 | 0.213313342 |
| 760 | G | D | 0.316786277 | 0.162885521 | 901 | S | R | 0.311953255 | 0.163461395 |
| 244 | Q | K | 0.316600083 | 0.246636704 | 393 | F | L | 0.311946018 | 0.192991506 |
| 238 | S | Y | 0.316596499 | 0.171458712 | 757 | L | P | 0.311927617 | 0.117197609 |
| 475 | F | L | 0.316549309 | 0.192939087 | 702 | R | G | 0.311688104 | 0.266620819 |
| 829 | K | N | 0.316494901 | 0.154808851 | 589 | K | R | 0.311588343 | 0.136320933 |
| 28 | M | I | 0.31630177 | 0.188404934 | 717 | G | R | 0.311565735 | 0.080863714 |
| 186 | G | A | 0.316262682 | 0.1767869 | 286 | T | S | 0.311321567 | 0.240949263 |
| 679 | R | G | 0.316180477 | 0.112760057 | 150 | P | T | 0.311291496 | 0.13427262 |
| 925 | A | G | 0.315901657 | 0.192750307 | 107 | I | L | 0.307707331 | 0.205313283 |
| 892 | A | P | 0.315901657 | 0.129374073 | 776 | T | A | 0.307705621 | 0.113209696 |
| 642 | E | A | 0.315758891 | 0.205380131 | 306 | L | V | 0.307515106 | 0.116397313 |
| 629 | E | G | 0.315702888 | 0.119743865 | 651 | P | T | 0.307457933 | 0.189846398 |
| 642 | E | G | 0.315673565 | 0.11044042 | 155 | F | Y | 0.307385155 | 0.165676404 |
| 104 | P | R | 0.315607101 | 0.202791238 | 229 | S | T | 0.307373154 | 0.086318269 |
| 807 | K | E | 0.315573228 | 0.117464708 | 517 | I | V | 0.307363772 | 0.108604289 |
| 599 | D | E | 0.315416693 | 0.115740153 | 334 | V | A | 0.306982037 | 0.139604112 |
| 578 | P | A | 0.311263999 | 0.106013626 | 614 | R | K | 0.306921623 | 0.187827913 |
| 41 | R | G | 0.311016733 | 0.286865829 | 824 | V | L | 0.306719384 | 0.210851946 |
| 781 | W | S | 0.310870839 | 0.281958829 | 723 | A | V | 0.306692766 | 0.140247988 |
| 382 | S | I | 0.310857774 | 0.22558917 | 711 | E | G | 0.306675894 | 0.224133351 |
| 723 | A | T | 0.310856537 | 0.118165477 | 499 | E | Q | 0.306671973 | 0.224590082 |
| 451 | A | G | 0.310527551 | 0.159640493 | 104 | P | S | 0.306640385 | 0.162249455 |
| 568 | P | L | 0.310447286 | 0.186724922 | 3 | I | L | 0.306608196 | 0.194776786 |
| 216 | G | S | 0.310362762 | 0.143843218 | 702 | R | K | 0.306541295 | 0.149431609 |
| 216 | G | R | 0.310272111 | 0.119909677 | 954 | K | E | 0.306525004 | 0.187285491 |
| 89 | Q | R | 0.310167676 | 0.139047602 | 842 | --- | KEL | 0.306410776 | 0.206532128 |
| 433 | K | R | 0.310161393 | 0.097615554 | 466 | G | C | 0.30635382 | 0.179163452 |
| 21 | KA | NC | 0.310061242 | 0.098851828 | 979 | ----- | VSSKD (SEQ | 0.306277048 | 0.179502088 |
| | | | | | | | ID NO: 3799) | | |
| | | | | | | | [stop] | | |
| 141 | L | P | 0.309573602 | 0.118441502 | 830 | K | | 0.306086752 | 0.154175951 |
| 425 | D | Y | 0.309531408 | 0.253195982 | 243 | Y | F | 0.306073033 | 0.15669665 |
| 579 | N | D | 0.309484128 | 0.137585893 | 88 | F | L | 0.305867737 | 0.156711191 |
| 825 | L | V | 0.309431153 | 0.160157183 | 149 | K | E | 0.305762803 | 0.092392237 |
| 464 | I | M | 0.309049855 | 0.208541437 | 102 | P | H | 0.305663323 | 0.198476248 |
| 710 | V | L | 0.309047105 | 0.126001585 | 554 | ---- | RFYT (SEQ | 0.305511625 | 0.122801047 |
| | | | | | | | ID NO: 3665) | | |
| 671 | D | H | 0.309035221 | 0.209514286 | 720 | - | R | 0.305347434 | 0.161540535 |
| 735 | R | P | 0.309028904 | 0.132025621 | 128 | A | G | 0.305254739 | 0.159245241 |
| 819 | A | G | 0.308778739 | 0.188847749 | 122 | L | P | 0.305222365 | 0.154910099 |
| 2 | E | G | 0.308512084 | 0.159248809 | 792 | P | S | 0.305214901 | 0.160903917 |
| 109 | Q | H | 0.308384304 | 0.180580793 | 312 | L | P | 0.305192803 | 0.183880511 |
| 66 | L | V | 0.308337109 | 0.160085063 | 299 | Q | [stop] | 0.305119863 | 0.096364942 |
| 93 | V | L | 0.308334538 | 0.186355769 | 668 | A | T | 0.305069729 | 0.135204642 |
| 621 | Y | [stop] | 0.308307714 | 0.182192979 | 962 | Q | R | 0.302114892 | 0.192863031 |
| 0 | M | L | 0.308276685 | 0.236934633 | 656 | G | S | 0.301941181 | 0.160658808 |
| 857 | K | E | 0.308118374 | 0.128063493 | 526 | L | P | 0.301907253 | 0.200130867 |
| 264 | L | I | 0.308089176 | 0.231951197 | 181 | V | L | 0.301627326 | 0.141701986 |
| 646 | S | T | 0.307934288 | 0.163215891 | 602 | S | G | 0.301374384 | 0.168690577 |
| 461 | S | T | 0.307923977 | 0.13026743 | 2 | E | K | 0.301361669 | 0.293245611 |
| 937 | S | N | 0.307902696 | 0.280386833 | 46 | N | S | 0.301357514 | 0.121526311 |
| 774 | Q | L | 0.30782826 | 0.179585187 | 71 | T | S | 0.301285774 | 0.182156883 |
| 427 | K | N | 0.307771318 | 0.212433986 | 887 | G | D | 0.301271887 | 0.117733719 |
| 422 | E | G | 0.307743696 | 0.21393123 | 121 | R | S | 0.301231571 | 0.167844846 |
| 639 | E | Q | 0.304680843 | 0.266883075 | 108 | D | V | 0.301094262 | 0.261979025 |
| 812 | C | [stop] | 0.304671385 | 0.223383408 | 979 | LE[stop]GS- | VSSKDLQA | 0.301043 | 0.222937332 |
| | | | | | | PGI (SEQ ID | (SEQ ID NO: | | |
| | | | | | | NO: 3278) | 3810)[stop] | | |
| 856 | -- | YK | 0.304562199 | 0.117931145 | 73 | Y | [stop] | 0.300976299 | 0.109164204 |
| 959 | ------- | ETWQSFY | 0.304562199 | 0.204359044 | 645 | D | H | 0.300832783 | 0.189820783 |
| | | (SEQ ID NO: | | | | | | | |
| | | 3403) | | | | | | | |
| 640 | R | [stop] | 0.304365031 | 0.131009317 | 972 | --- | VWK | 0.300386808 | 0.146545616 |
| 968 | KL | S[stop] | 0.304328899 | 0.221090558 | 127 | F | S | 0.300342022 | 0.146847301 |
| 24 | K | N | 0.304215048 | 0.239991354 | 571 | V | A | 0.300337937 | 0.156010497 |
| 858 | R | T | 0.304052714 | 0.1448623 | 386 | D | N | 0.300273532 | 0.259491112 |
| 530 | L | M | 0.303970715 | 0.250168829 | 381 | L | M | 0.300116697 | 0.157006178 |
| 269 | S | R | 0.303928294 | 0.209763505 | 493 | P | A | 0.299995588 | 0.227049942 |
| 251 | Q | E | 0.303459913 | 0.190095434 | 199 | H | R | 0.299830107 | 0.074234175 |
| 340 | E | Q | 0.30343193 | 0.10804688 | 642 | E | [stop] | 0.299768631 | 0.20842894 |
| 623 | - | R | 0.303430789 | 0.233394445 | 352 | K | [stop] | 0.299555207 | 0.106916877 |
| 880 | D | Y | 0.30324465 | 0.244720194 | 314 | I | V | 0.299339024 | 0.237860572 |
| 223 | P | A | 0.303031527 | 0.177373299 | 696 | S | T | 0.299269551 | 0.19370537 |
| 899 | R | T | 0.302967154 | 0.112177355 | 554 | R | G | 0.299260223 | 0.263070996 |
| 60 | N | D | 0.30295183 | 0.177064719 | 413 | W | S | 0.298889603 | 0.120871006 |
| 966 | R | S | 0.302926375 | 0.099801177 | 973 | W | [stop] | 0.298886432 | 0.173734887 |
| 687 | P | A | 0.302859855 | 0.188291569 | 1 | Q | [stop] | 0.298848883 | 0.253324527 |
| 821 | Y | C | 0.302780706 | 0.154234626 | 59 | S | G | 0.298416382 | 0.178538741 |
| 628 | D | Y | 0.302709978 | 0.176578494 | 717 | G | [stop] | 0.298317755 | 0.217662606 |
| 952 | -------- | TDKRAFVE | 0.302629733 | 0.089246659 | 348 | C | S | 0.298274049 | 0.13599769 |
| | | (SEQ ID NO: | | | | | | | |
| | | 3741) | | | | | | | |
| 540 | L | V | 0.302623885 | 0.094608809 | 707 | A | G | 0.298173789 | 0.189062395 |
| 855 | R | T | 0.302608606 | 0.19469877 | 345 | D | Y | 0.295298688 | 0.153403354 |
| 59 | S | I | 0.302606901 | 0.165051866 | 469 | E | G | 0.295269456 | 0.193145904 |
| 272 | G | D | 0.302541592 | 0.185286895 | 495 | A | T | 0.295248074 | 0.179130836 |
| 284 | P | H | 0.302498547 | 0.213421981 | 929 | A | G | 0.295233981 | 0.250007265 |
| 342 | -- | TS | 0.302413033 | 0.240972915 | 435 | I | T | 0.2952095 | 0.10707736 |
| 43 | R | W | 0.302283296 | 0.149981215 | 586 | A | T | 0.295123473 | 0.125804414 |
| 760 | G | A | 0.302207311 | 0.130376601 | 627 | Q | R | 0.295089748 | 0.147312376 |
| 766 | K | N | 0.302181165 | 0.136382512 | 17 | S | I | 0.295022842 | 0.203345294 |
| 478 | CE | AQ | 0.298056287 | 0.28697996 | 96 | M | V | 0.29492941 | 0.118289949 |
| 915 | G | A | 0.298020743 | 0.21282862 | 83 | V | M | 0.294841632 | 0.151911965 |
| 969 | L | M | 0.297993119 | 0.288243926 | 721 | K | [stop] | 0.294783263 | 0.121804362 |
| 953 | D | V | 0.297929214 | 0.145206254 | 550 | F | S | 0.294772324 | 0.160417343 |
| 485 | W | G | 0.297911414 | 0.242181721 | 538 | G | A | 0.29474804 | 0.174345187 |
| 676 | P | A | 0.297863971 | 0.089640148 | 462 | F | L | 0.294742725 | 0.14185505 |
| 4 | K | T | 0.297828559 | 0.161108285 | 822 | D | H | 0.294658575 | 0.162957386 |
| 631 | A | G | 0.297777083 | 0.103836414 | 213 | QI | PV | 0.294575907 | 0.193654425 |
| 250 | H | P | 0.29766948 | 0.081415922 | 658 | D | N | 0.294502464 | 0.107952026 |
| 11 | - | R | 0.29755173 | 0.242218951 | 309 | W | S | 0.294338009 | 0.284836107 |
| 274 | A | T | 0.297540582 | 0.172279995 | 835 | W | C | 0.294317109 | 0.120763755 |
| 918 | T | K | 0.297381988 | 0.249593921 | 607 | S | Y | 0.294194742 | 0.192145848 |
| 43 | R | L | 0.297375059 | 0.247052829 | 853 | Y | [stop] | 0.294188525 | 0.116100881 |
| 51 | P | A | 0.29736536 | 0.241677851 | 895 | L | M | 0.294152124 | 0.189733578 |
| 64 | A | T | 0.297190007 | 0.136022098 | 298 | AQ | DR | 0.294067945 | 0.080730567 |
| 617 | E | Q | 0.297156994 | 0.256789508 | 221 | S | T | 0.293988985 | 0.161830985 |
| 468 | | K | 0.297121715 | 0.218726347 | 854 | ----- | NRYKRQ | 0.29389502 | 0.164228467 |
| | | | | | | | (SEQ ID NO: | | |
| | | | | | | | 3597) | | |
| 705 | Q | [stop] | 0.297097391 | 0.129530594 | 184 | --- | SLG | 0.29389502 | 0.133943716 |
| 538 | G | D | 0.297030166 | 0.143641253 | 24 | K | E | 0.293893146 | 0.087429384 |
| 697 | Y | [stop] | 0.29694611 | 0.165401562 | 903 | R | T | 0.293855808 | 0.156130706 |
| 30 | T | N | 0.296922856 | 0.20113666 | 649 | I | M | 0.293844709 | 0.213121389 |
| 374 | Q | E | 0.296916876 | 0.294201034 | 646 | S | N | 0.293718938 | 0.053702828 |
| 429 | E | G | 0.296692622 | 0.12956891 | 751 | M | T | 0.293692865 | 0.188828745 |
| 617 | E | G | 0.296673186 | 0.100617287 | 138 | V | A | 0.293692865 | 0.172441917 |
| 174 | P | L | 0.296325925 | 0.125090192 | 421 | W | R | 0.293643119 | 0.202965718 |
| 476 | C | W | 0.296243077 | 0.108583652 | 891 | E | D | 0.290888227 | 0.199229012 |
| 536 | K | [stop] | 0.296174047 | 0.204485045 | 663 | I | T | 0.290884576 | 0.159824412 |
| 340 | E | [stop] | 0.296106359 | 0.228363644 | 86 | E | G | 0.290735509 | 0.164271816 |
| 263 | N | S | 0.295761788 | 0.153417105 | 950 | ------- | GNTDKRA | 0.290646329 | 0.08439848 |
| | | | | | | | (SEQ ID NO: | | |
| | | | | | | | 3447) | | |
| 292 | A | D | 0.295588873 | 0.132003236 | 910 | V | A | 0.290614659 | 0.192165123 |
| 524 | K | E | 0.295588726 | 0.123024834 | 130 | S | R | 0.290579337 | 0.126556505 |
| 252 | K | E | 0.295509892 | 0.130412924 | 286 | T | A | 0.290569747 | 0.161258253 |
| 360 | D | H | 0.295426779 | 0.169820671 | 412 | D | Y | 0.290563856 | 0.192946257 |
| 771 | A | T | 0.295409018 | 0.21146028 | 390 | G | C | 0.290531408 | 0.226107283 |
| 960 | T | S | 0.295303172 | 0.200733126 | 96 | M | T | 0.290483084 | 0.117441458 |
| 885 | T | A | 0.293639992 | 0.136222429 | 796 | Y | F | 0.290480726 | 0.145066767 |
| 372 | K | N | 0.293601801 | 0.159631501 | 617 | E | [stop] | 0.290459043 | 0.254049857 |
| 899 | R | W | 0.293409271 | 0.197663789 | 520 | K | Q | 0.290432231 | 0.149193863 |
| 323 | Q | R | 0.293396269 | 0.187618952 | 238 | S | C | 0.29036146 | 0.125809391 |
| 787 | A | V | 0.293181255 | 0.111256021 | 510 | K | N | 0.290307315 | 0.121616244 |
| 97 | S | G | 0.29311892 | 0.120983434 | 751 | M | I | 0.290086322 | 0.117481113 |
| 523 | V | A | 0.293107836 | 0.144403198 | 764 | Q | E | 0.290043861 | 0.213865459 |
| 606 | GS | -A | 0.293095145 | 0.176419666 | 239 | F | L | 0.290032145 | 0.120563078 |
| 647 | S | G | 0.293070849 | 0.180316262 | 750 | A | S | 0.290021488 | 0.169783417 |
| 401 | L | M | 0.293059235 | 0.238931791 | 509 | S | N | 0.290010303 | 0.173158694 |
| 706 | A | T | 0.293004089 | 0.157196701 | 791 | L | V | 0.28993006 | 0.240441646 |
| 167 | I | M | 0.292976512 | 0.174804994 | 976 | A | P | 0.289917569 | 0.129909297 |
| 239 | F | Y | 0.292846447 | 0.244049066 | 970 | K | E | 0.289792346 | 0.088055606 |
| 532 | I | M | 0.292790974 | 0.132047771 | 370 | G | S | 0.289754414 | 0.116500268 |
| 362 | K | N | 0.292779584 | 0.196868197 | 229 | S | I | 0.289718863 | 0.192569781 |
| 531 | I | F | 0.292690193 | 0.245999103 | 126 | G | S | 0.289695476 | 0.136718855 |
| 551 | E | D | 0.292676692 | 0.177028816 | 39 | D | H | 0.28966543 | 0.205820796 |
| 366 | Q | R | 0.292637285 | 0.233099785 | 541 | R | W | 0.289647451 | 0.149474595 |
| 45 | E | K | 0.292602703 | 0.135241306 | 963 | S | R | 0.289642486 | 0.119359764 |
| 170 | S | P | 0.292487757 | 0.117055288 | 614 | R | G | 0.289631701 | 0.096593744 |
| 522 | -------- | GVKKLNLY | 0.292477218 | 0.205588046 | 903 | R | K | 0.289598509 | 0.276955136 |
| | | (SEQ ID NO: | | | | | | | |
| | | 3455) | | | | | | | |
| 184 | S | T | 0.292461578 | 0.171099938 | 700 | K | E | 0.289582689 | 0.146563937 |
| 256 | K | R | 0.292459664 | 0.134546625 | 176 | A | T | 0.289565984 | 0.071489526 |
| 898 | K | R | 0.292371281 | 0.233917307 | 862 | V | L | 0.28755723 | 0.122530143 |
| 687 | ------ | PTHILR (SEQ | 0.292237604 | 0.252992689 | 376 | A | D | 0.287488687 | 0.149852687 |
| | | ID NO: 3627) | | | | | | | |
| 499 | E | [stop] | 0.292180944 | 0.205912614 | 717 | G | A | 0.287475979 | 0.138371481 |
| 439 | E | [stop] | 0.291789527 | 0.178224776 | 871 | R | G | 0.287423469 | 0.12544588 |
| 286 | T | I | 0.291597253 | 0.134630039 | 779 | E | [stop] | 0.287388451 | 0.214465092 |
| 326 | K | R | 0.291167908 | 0.130858044 | 659 | R | Q | 0.287382153 | 0.188389105 |
| 309 | W | C | 0.291117426 | 0.126634127 | 688 | T | S | 0.2872606 | 0.18090055 |
| 141 | L | V | 0.291053469 | 0.125358393 | 450 | A | G | 0.287222025 | 0.226851871 |
| 599 | D | H | 0.290990101 | 0.194898673 | 608 | L | P | 0.287206606 | 0.153956956 |
| 714 | R | G | 0.289551118 | 0.131217053 | 74 | T | A | 0.28708898 | 0.151009591 |
| 849 | Q | E | 0.289450204 | 0.14256548 | 101 | Q | H | 0.287075864 | 0.127870371 |
| 861 | V | L | 0.289424991 | 0.184715842 | 168 | L | M | 0.287051161 | 0.164606192 |
| 227 | A | S | 0.289407395 | 0.147147965 | 522 | G | A | 0.286889556 | 0.191392288 |
| 337 | Q | E | 0.289400311 | 0.154536453 | 158 | -- | CN | 0.286856801 | 0.104191954 |
| 282 | P | Q | 0.289371748 | 0.241776764 | 822 | D | Y | 0.286792384 | 0.216414998 |
| 147 | ----- | KGKPH (SEQ | 0.289327222 | 0.167067239 | 31 | LL | PV | 0.286704233 | 0.167404084 |
| | | ID NO: 3494) | | | | | | | |
| 215 | -------- | GGNSCASG | 0.28926976 | 0.113347286 | 753 | ------ | IFENLS (SEQ | 0.286664247 | 0.204891377 |
| | | (SEQ ID NO: | | | | | ID NO: 3474) | | |
| | | 3432) | | | | | | | |
| 615 | - | Q | 0.288918789 | 0.138819471 | 894 | ---- | SLLK (SEQ | 0.286588033 | 0.088926565 |
| | | | | | | | ID NO: 3719) | | |
| 148 | ------- | GKPHTNY | 0.288918789 | 0.145077971 | 443 | S | R | 0.286575868 | 0.16053834 |
| | | (SEQ ID NO: | | | | | | | |
| | | 3438) | | | | | | | |
| 70 | L | V | 0.288897546 | 0.141249384 | 813 | G | S | 0.286517663 | 0.166687094 |
| 131 | Q | H | 0.28889109 | 0.089984222 | 545 | I | T | 0.28643634 | 0.175437623 |
| 417 | Y | [stop] | 0.288830461 | 0.139069155 | 43 | R | G | 0.286322337 | 0.211707784 |
| 917 | E | Q | 0.288684907 | 0.209421131 | 671 | D | G | 0.28629192 | 0.163952723 |
| 681 | K | R | 0.288657171 | 0.188212382 | 501 | S | T | 0.286282753 | 0.120251174 |
| 824 | --- | VLE | 0.288568311 | 0.142383803 | 729 | L | M | 0.286200559 | 0.141100837 |
| 757 | L | M | 0.288547614 | 0.138199941 | 264 | L | F | 0.28603772 | 0.148836446 |
| 683 | S | P | 0.288449161 | 0.100064584 | 613 | G | S | 0.285821749 | 0.213295055 |
| 879 | N | D | 0.288359669 | 0.112916417 | 806 | S | P | 0.285754508 | 0.139734573 |
| 87 | EF | AV | 0.28833835 | 0.157423397 | 251 | Q | R | 0.285704309 | 0.129794167 |
| 623 | R | M | 0.288312668 | 0.180378091 | 503 | L | P | 0.285623626 | 0.150765257 |
| 360 | D | G | 0.288240177 | 0.1450193 | 544 | K | N | 0.285528499 | 0.105740594 |
| 469 | E | D | 0.288213424 | 0.169330277 | 685 | G | S | 0.285482686 | 0.116956671 |
| 488 | D | H | 0.288056714 | 0.224399768 | 66 | L | P | 0.285241304 | 0.178235911 |
| 832 | A | D | 0.28797086 | 0.133987122 | 713 | R | [stop] | 0.281751627 | 0.150509506 |
| 331 | F | L | 0.287898632 | 0.125465761 | 759 | R | I | 0.281715415 | 0.207490665 |
| 880 | D | N | 0.287796432 | 0.265861692 | 103 | A | D | 0.281654023 | 0.156258821 |
| 813 | G | V | 0.28764847 | 0.18793522 | 352 | K | R | 0.281644749 | 0.090972271 |
| 125 | S | R | 0.287612867 | 0.078156909 | 23 | G | D | 0.281613067 | 0.110087313 |
| 315 | G | V | 0.287582891 | 0.216366011 | 490 | R | I | 0.28158749 | 0.189684 |
| 348 | C | [stop] | 0.285167016 | 0.232120541 | 534 | Y | C | 0.281578683 | 0.19797794 |
| 615 | V | L | 0.285139566 | 0.138644746 | 728 | N | K | 0.281567938 | 0.122533743 |
| 34 | R | K | 0.285068253 | 0.155629412 | 218 | S | G | 0.28156304 | 0.0827746 |
| 606 | G | D | 0.284708065 | 0.131937418 | 131 | Q | K | 0.28143462 | 0.261996702 |
| 564 | G | R | 0.284584869 | 0.153328649 | 117 | D | Y | 0.281261616 | 0.150312544 |
| 767 | R | G | 0.284520477 | 0.167110905 | 809 | C | S | 0.281246687 | 0.119977311 |
| 459 | K | N | 0.284319069 | 0.144116629 | 899 | R | S | 0.281103794 | 0.115069396 |
| 100 | A | G | 0.284064196 | 0.232698011 | 192 | A | P | 0.281083951 | 0.125030936 |
| 182 | T | S | 0.284017418 | 0.165066704 | 913 | N | S | 0.280977138 | 0.259159821 |
| 552 | A | P | 0.28399207 | 0.192922882 | 232 | C | S | 0.28083211 | 0.170644437 |
| 874 | E | [stop] | 0.283924403 | 0.212096559 | 928 | I | L | 0.280808974 | 0.249623753 |
| 656 | G | V | 0.283837412 | 0.096364514 | 495 | A | G | 0.280579997 | 0.166279564 |
| 527 | N | D | 0.283828964 | 0.095606466 | 917 | ----- | ETHAA (SEQ | 0.280544768 | 0.259917773 |
| | | | | | | | ID NO: 3399) | | |
| 560 | N | D | 0.283827293 | 0.131100485 | 85 | W- | LS | 0.280472053 | 0.101385815 |
| 518 | W | [stop] | 0.283768829 | 0.144873432 | 344 | W | [stop] | 0.280246002 | 0.139860723 |
| 900 | F | Y | 0.283754684 | 0.18210141 | 493 | P | H | 0.280219202 | 0.225933372 |
| 485 | W | C | 0.283722783 | 0.101623525 | 189 | G | A | 0.28010846 | 0.181165246 |
| 528 | L | M | 0.283582823 | 0.241404553 | 565 | E | G | 0.28010846 | 0.126376781 |
| 463 | V | L | 0.283409253 | 0.174572622 | 944 | Q | R | 0.279992746 | 0.221800854 |
| 938 | Q | R | 0.283399277 | 0.159588016 | 674 | G | A | 0.27982066 | 0.112736684 |
| 809 | C | R | 0.2832933 | 0.140866937 | 45 | E | V | 0.279758496 | 0.126165976 |
| 765 | G | V | 0.283226034 | 0.181883423 | 281 | P | A | 0.27973122 | 0.169207983 |
| 253 | V | E | 0.283192966 | 0.158310209 | 828 | L | P | 0.279653349 | 0.165044194 |
| 745 | A | D | 0.283094632 | 0.139036808 | 460 | A | D | 0.27950426 | 0.185233285 |
| 739 | R | S | 0.283000418 | 0.086394522 | 539 | K | R | 0.279423784 | 0.231876099 |
| 262 | A | D | 0.282981572 | 0.21883829 | 62 | S | G | 0.279325036 | 0.105769252 |
| 75 | E | D | 0.282861668 | 0.096240394 | 883 | S | T | 0.278909433 | 0.17133128 |
| 122 | L | V | 0.28282995 | 0.142431105 | 166 | --- | LIL | 0.27890183 | 0.114735325 |
| 427 | K | R | 0.282689541 | 0.126741896 | 553 | N | K | 0.276534729 | 0.129122139 |
| 472 | K | E | 0.282354225 | 0.243592384 | 500 | N | K | 0.276479484 | 0.075342066 |
| 69 | L | V | 0.282311609 | 0.233097353 | 796 | Y | [stop] | 0.276459628 | 0.151040972 |
| 128 | A | D | 0.282136746 | 0.144684711 | 313 | K | E | 0.276424062 | 0.141250225 |
| 240 | L | P | 0.282112821 | 0.187484636 | 184 | S | R | 0.276360484 | 0.093462218 |
| 840 | N | D | 0.28205862 | 0.169019904 | 770 | M | V | 0.276349013 | 0.177344184 |
| 496 | I | L | 0.281766947 | 0.156440465 | 30 | T | S | 0.27626759 | 0.074607362 |
| 445 | D | N | 0.27879438 | 0.120139275 | 887 | G | C | 0.276203171 | 0.205245818 |
| 121 | R | G | 0.278752599 | 0.152495589 | 885 | T | S | 0.276162821 | 0.125136939 |
| 66 | LN | PV | 0.278503247 | 0.058556198 | 372 | K | E | 0.2761455 | 0.186164615 |
| 603 | ------- | LETGSLK | 0.278503247 | 0.20379117 | 161 | S | F | 0.276099268 | 0.101256778 |
| | | (SEQ ID NO: | | | | | | | |
| | | 3545) | | | | | | | |
| 225 | G | [stop] | 0.278489806 | 0.182580993 | 280 | LP | PV | 0.2760948 | 0.15312325 |
| 175 | --- | EAN | 0.278488851 | 0.117512649 | 118 | G | A | 0.276069076 | 0.158472607 |
| 274 | A | S | 0.278435433 | 0.213434648 | 945 | T | S | 0.275967844 | 0.217091948 |
| 870 | D | G | 0.278347965 | 0.136371883 | 597 | W | S | 0.275959763 | 0.205648781 |
| 683 | S | T | 0.278234202 | 0.119170388 | 700 | K | [stop] | 0.275943939 | 0.231744011 |
| 792 | P | H | 0.277909356 | 0.196357382 | 654 | L | M | 0.275895098 | 0.222206287 |
| 18 | N | R | 0.277904726 | 0.144376969 | 34 | R | I | 0.275728667 | 0.262529033 |
| 484 | K | R | 0.277812806 | 0.156918996 | 650 | K | N | 0.275727906 | 0.092682765 |
| 51 | P | H | 0.27780081 | 0.207949147 | 347 | V | D | 0.275634849 | 0.162043607 |
| 549 | A | D | 0.277618034 | 0.184792104 | 701 | Q | E | 0.275445666 | 0.129639485 |
| 285 | H | Q | 0.277595201 | 0.164383067 | 221 | S | P | 0.275424064 | 0.253543179 |
| 772 | E | [stop] | 0.277569205 | 0.252009775 | 902 | H | Y | 0.275413846 | 0.238626124 |
| 233 | M | T | 0.277522281 | 0.101460422 | 408 | K | N | 0.275278915 | 0.187758493 |
| 677 | ------- | LSRFKDS | 0.277439144 | 0.176461932 | 410 | G | R | 0.275207307 | 0.148329245 |
| | | (SEQ ID NO: | | | | | | | |
| | | 3578) | | | | | | | |
| 444 | E | D | 0.277438575 | 0.185715982 | 202 | R | T | 0.27519939 | 0.225294793 |
| 287 | K | R | 0.277424076 | 0.122002352 | 190 | Q | H | 0.275101911 | 0.155497318 |
| 86 | E | Q | 0.277422525 | 0.267475322 | 296 | V | A | 0.274868513 | 0.216028266 |
| 650 | K | R | 0.277338051 | 0.1661601 | 176 | A | V | 0.274754076 | 0.101747221 |
| 119 | N | K | 0.2772012 | 0.097660237 | 16 | D | V | 0.274707044 | 0.080710216 |
| 419 | E | D | 0.27717758 | 0.091079949 | 338 | A | G | 0.274649181 | 0.21549192 |
| 849 | Q | H | 0.277146577 | 0.10057266 | 908 | K | [stop] | 0.274631009 | 0.235774306 |
| 745 | A | P | 0.277094424 | 0.180486538 | 745 | A | T | 0.274596368 | 0.139876086 |
| 895 | L | V | 0.277059576 | 0.147621158 | 582 | I | T | 0.274539152 | 0.136455089 |
| 200 | V | R | 0.276947529 | 0.109871945 | 73 | Y | H | 0.274522926 | 0.183155681 |
| 491 | G | A | 0.276923451 | 0,236639042 | 525 | ------ | KLNLYL | 0.272179534 | 0.127115618 |
| | | | | | | | (SEQ ID NO: | | |
| | | | | | | | 3512) | | |
| 437 | L | P | 0.276817656 | 0.127643327 | 178 | D | H | 0.27217863 | 0.114858223 |
| 794 | K | E | 0.276808052 | 0.108760175 | 186 | G | S | 0.272004663 | 0.206440397 |
| 609 | K | E | 0.274518342 | 0.096584602 | 797 | LS | PV | 0.271846299 | 0.116235959 |
| 148 | ----- | GKPHT (SEQ | 0.274483854 | 0.138944547 | 434 | H | L | 0.271775834 | 0.108387354 |
| | | ID NO: 3436) | | | | | | | |
| 269 | S | I | 0.274483065 | 0.167999753 | 124 | S | C | 0.271634239 | 0.201362524 |
| 600 | L | P | 0.274446407 | 0.156944314 | 687 | ---- | PTHI (SEQ ID | 0.271046382 | 0.217907583 |
| | | | | | | | NO: 3625) | | |
| 609 | K | N | 0.274296988 | 0.098675974 | 626 | R | I | 0.271037385 | 0.191496316 |
| 548 | E | G | 0.274291628 | 0.174184065 | 717 | G | V | 0.271024109 | 0.162847575 |
| 282 | P | R | 0.274223113 | 0.269615449 | 534 | Y | [stop] | 0.270681224 | 0.104188898 |
| 743 | Y | N | 0.274041951 | 0.169744437 | 150 | P | H | 0.270599643 | 0.192362809 |
| 273 | LA | PV | 0.273953381 | 0.083004597 | 552 | A | S | 0.270597368 | 0.181876059 |
| 241 | ----- | TKYQD (SEQ | 0.273953381 | 0.041697608 | 150 | P | S | 0.270581156 | 0.14794261 |
| | | ID NO: 3752) | | | | | | | |
| 752 | LI | PV | 0.273953381 | 0.179521275 | 270 | A | S | 0.270550408 | 0.145246028 |
| 500 | ----- | NSILD (SEQ | 0.273953381 | 0.096079618 | 563 | S | Y | 0.270533409 | 0.17681632 |
| | | ID NO: 3598) | | | | | | | |
| 88 | FQ | DR | 0.273953381 | 0.132934109 | 664 | --- | PAV | 0.270462826 | 0.090794222 |
| 548 | E | K | 0.273785339 | 0.140999456 | 97 | S | I | 0.270410385 | 0.155670382 |
| 758 | S | T | 0.273170088 | 0.17814745 | 64 | A | D | 0.270367942 | 0.13574281 |
| 884 | W | S | 0.27315778 | 0.127540825 | 143 | Q | E | 0.27021122 | 0.220203083 |
| 258 | E | D | 0.273147573 | 0.172394328 | 686 | N | I | 0.270089028 | 0.228432562 |
| 720 | R | M | 0.272984313 | 0.209562405 | 544 | K | [stop] | 0.270051777 | 0.124983342 |
| 217 | N | H | 0.272871217 | 0.212149421 | 537 | G | A | 0.270050779 | 0.18424231 |
| 0 | M | R | 0.272866831 | 0.105028991 | 902 | H | L | 0.269853978 | 0.238618549 |
| 376 | A | G | 0.27284261 | 0.107816996 | 361 | G | A | 0.269774718 | 0.191146018 |
| 221 | S | C | 0.272816553 | 0.204562414 | 963 | S | C | 0.269617744 | 0.20243244 |
| 691 | LR | PV | 0.272779276 | 0.168092844 | 965 | Y | H | 0.26944455 | 0.246260675 |
| 796 | YL | DR | 0.272779276 | 0.144849416 | 66 | --- | LNK | 0.269318761 | 0.181427468 |
| 439 | ---- | EERR (SEQ | 0.272779276 | 0.117493254 | 959 | ----- | ETWQS (SEQ | 0.269318761 | 0.133778085 |
| | | ID NO: 3381) | | | | | ID NO: 3402) | | |
| 383 | S | N | 0.272651878 | 0.203030872 | 509 | ----- | SKQYN (SEQ | 0.269239232 | 0.199612231 |
| | | | | | | | ID NO: 3712) | | |
| 603 | L | M | 0.272615876 | 0.2046327 | 32 | L | I | 0.269033673 | 0.109933858 |
| 183 | Y | H | 0.27230417 | 0.167987777 | 913 | N | I | 0.265873279 | 0.228181021 |
| 858 | R | K | 0.272264159 | 0.162833579 | 775 | Y | S | 0.265844485 | 0.132207982 |
| 209 | K | N | 0.269020729 | 0.109971766 | 678 | S | R | 0.265770435 | 0.147977027 |
| 48 | R | [stop] | 0.268939151 | 0.082435645 | 602 | S | R | 0.265750704 | 0.118408744 |
| 466 | - | T | 0.268825688 | 0.095723888 | 121 | R | T | 0.265718915 | 0.126781949 |
| 45 | E | Q | 0.268733142 | 0.139266278 | 818 | S | R | 0.265623217 | 0.145609734 |
| 843 | E | Q | 0.268599201 | 0.195661988 | 798 | S | C | 0.265584497 | 0.073889024 |
| 643 | V | L | 0.268577714 | 0.156052892 | 864 | ------ | DLSVEL | 0.265506357 | 0.19885122 |
| | | | | | | | (SEQ ID NO: | | |
| | | | | | | | 3365) | | |
| 285 | H | R | 0.268299231 | 0.21489701 | 373 | R | G | 0.265364174 | 0.162678423 |
| 317 | D | G | 0.268047511 | 0.116283826 | 803 | Q | E | 0.265269725 | 0.202509841 |
| 195 | F | L | 0.268045884 | 0.108480308 | 628 | D | E | 0.265261641 | 0.142156395 |
| 590 | R | K | 0.267781681 | 0.208536761 | 194 | D | N | 0.265249363 | 0.155857424 |
| 180 | L | V | 0.267694655 | 0.240305187 | 336 | R | I | 0.2651284 | 0.181377392 |
| 21 | KA | TV | 0.267470584 | 0.147038119 | 602 | S | I | 0.265065039 | 0.204267576 |
| 210 | P | H | 0.267434518 | 0.190772597 | 34 | R | S | 0.265026085 | 0.223416007 |
| 612 | N | S | 0.267419306 | 0.129882451 | 775 | Y | N | 0.264899495 | 0.150356822 |
| 440 | E | G | 0.267419306 | 0.166870392 | 647 | ---- | SNIK (SEQ ID | 0.264896362 | 0.152108713 |
| | | | | | | | NO: 3725) | | |
| 651 | P | L | 0.267350724 | 0.179171164 | 369 | A | G | 0.264866639 | 0.127314344 |
| 686 | ------- | NPTHILR | 0.267281547 | 0.145940038 | 407 | KKHGEDWG | RSTARTGA | 0.26465494 | 0.11425501 |
| | | (SEQ ID NO: | | | | (SEQ ID NO: | (SEQ ID NO: | | |
| | | 3595) | | | | 3269) | 3688) | | |
| 56 | Q | E | 0.267209421 | 0.156465006 | 117 | D | H | 0.264598341 | 0.092643909 |
| 656 | G | D | 0.267197717 | 0.143131022 | 149 | K | R | 0.26429667 | 0.254633892 |
| 591 | Q | E | 0.267046259 | 0.172628923 | 624 | R | S | 0.264277774 | 0.09593797 |
| 771 | A | P | 0.266971248 | 0.20146384 | 526 | L | M | 0.26419728 | 0.176624184 |
| 667 | I | N | 0.266893998 | 0.140849994 | 671 | D | N | 0.264084519 | 0.212711081 |
| 333 | L | P | 0.26683779 | 0.202160591 | 572 | N | K | 0.264075863 | 0.218490453 |
| 168 | L | V | 0.266833554 | 0.09646076 | 949 | T | S | 0.263657544 | 0.110498861 |
| 43 | R | P | 0.266528412 | 0.166392391 | 20 | KKA | T-V | 0.263583848 | 0.126615658 |
| 76 | M | T | 0.26642278 | 0.06437874 | 56 | Q | R | 0.263561421 | 0.151855491 |
| 85 | WE | CC | 0.266335966 | 0.095081027 | 492 | K | N | 0.263524564 | 0.121563708 |
| 784 | A | D | 0.266225364 | 0.186318048 | 315 | G | D | 0.26350398 | 0.250984577 |
| 179 | E | G | 0.266200643 | 0.159572948 | 440 | E | [stop] | 0.260572941 | 0.226197983 |
| 282 | P | T | 0.266142294 | 0.234821238 | 245 | D | Y | 0.260411841 | 0.171518027 |
| 505 | I | V | 0.266033676 | 0.153318009 | 838 | T | A | 0.260310871 | 0.127668195 |
| 884 | W | C | 0.265892315 | 0.146379991 | 510 | K | E | 0.260303511 | 0.170827119 |
| 705 | Q | L | 0.265873279 | 0.218762249 | 885 | T | I | 0.260229119 | 0.18213929 |
| 625 | T | S | 0.263431268 | 0.11997699 | 606 | G | C | 0.260187776 | 0.249968408 |
| 657 | I | S | 0.26332391 | 0.140695845 | 298 | A | P | 0.260175418 | 0.137767012 |
| 688 | T | R | 0.26332192 | 0.129910161 | 31 | L | R | 0.260094537 | 0.205569477 |
| 835 | W | R | 0.263224631 | 0.136063076 | 19 | T | I | 0.259989986 | 0.207028692 |
| 903 | R | S | 0.263145681 | 0.157044964 | 886 | K | R | 0.259901164 | 0.087667222 |
| 876 | S | T | 0.262876961 | 0.112192073 | 817 | T | S | 0.259831477 | 0.054519088 |
| 468 | K | R | 0.262863102 | 0.120169191 | 901 | S | T | 0.259815097 | 0.082797155 |
| 590 | --- | RQG | 0.26279648 | 0.125412364 | 343 | W | S | 0.259761267 | 0.144643456 |
| 912 | L | R | 0.262679132 | 0.194562045 | 25 | T | R | 0.259617038 | 0.188030957 |
| 222 | G | R | 0.262575495 | 0.121179798 | 238 | S | P | 0.259597922 | 0.12796144 |
| 379 | P | A | 0.262556362 | 0.200217288 | 343 | W | R | 0.259570669 | 0.092335686 |
| 7 | N | Y | 0.262545332 | 0.249153444 | 317 | D | Y | 0.259540606 | 0.174340169 |
| 514 | C | R | 0.262528328 | 0.153764358 | 347 | ------ | VCNVICK | 0.259425173 | 0.186479916 |
| | | | | | | | (SEQ ID NO: | | |
| | | | | | | | 3770) | | |
| 964 | -- | FY | 0.262491519 | 0.18918584 | 606 | G | S | 0.259379927 | 0.201078104 |
| 951 | N | I | 0.262433241 | 0.181173796 | 879 | N | S | 0.259300679 | 0.19356618 |
| 738 | A | S | 0.262344275 | 0.213159289 | 784 | A | S | 0.259182688 | 0.192685039 |
| 109 | Q | K | 0.262161279 | 0.235829587 | 48 | R | I | 0.259088713 | 0.132594855 |
| 371 | Y | C | 0.262089785 | 0.121531872 | 112 | L | M | 0.25908476 | 0.122948809 |
| 62 | S | I | 0.262062515 | 0.217469036 | 181 | V | A | 0.259030426 | 0.153412207 |
| 967 | K | N | 0.261999761 | 0.11991933 | 567 | V | M | 0.258972858 | 0.206147057 |
| 395 | R | T | 0.261975414 | 0.202071604 | 787 | A | P | 0.258909575 | 0.199316536 |
| 546 | K | E | 0.261933935 | 0.196957538 | 741 | --- | LLY | 0.258835623 | 0.170116186 |
| 473 | D | H | 0.26183541 | 0.210514432 | 280 | -- | LP | 0.258711013 | 0.142341042 |
| 422 | | ERIDKKV | 0.261766763 | 0.175889641 | 639 | ------- | ERREVLD | 0.258711013 | 0.096645952 |
| | | (SEQ ID NO: | | | | | (SEQ ID NO: | | |
| | | 3393) | | | | | 3395) | | |
| 661 | E | D | 0.261685468 | 0.21738252 | 11 | RR | AS | 0.258711013 | 0.198257452 |
| 807 | K | N | 0.261631077 | 0.137745855 | 660 | G | V | 0.258707306 | 0.163939116 |
| 495 | A | P | 0.261336035 | 0.145111761 | 519 | ----- | QKDGVK | 0.255711118 | 0.090066635 |
| | | | | | | | (SEQ ID NO: | | |
| | | | | | | | 3641) | | |
| 474 | E | V | 0.261129255 | 0.1424745 | 977 | V | E | 0.255573788 | 0.223531947 |
| 100 | A | V | 0.261042682 | 0.097040591 | 448 | S | P | 0.255534334 | 0.216106849 |
| 660 | G | A | 0.260992911 | 0.257791059 | 872 | ---- | LSEE (SEQ | 0.255312236 | 0.130213196 |
| | | | | | | | ID NO: 3572) | | |
| 613 | G | V | 0.260991628 | 0.142830183 | 534 | -Y | DS | 0.255312236 | 0.080703663 |
| 356 | --- | EKK | 0.260606313 | 0.08939761 | 765 | -- | GK | 0.255312236 | 0.10865158 |
| 419 | E | R | 0.260606313 | 0.127113021 | 28 | MK | C- | 0.255312236 | 0.091611028 |
| 62 | S | N | 0.258582734 | 0.206139171 | 826 | EK | DR | 0.255312236 | 0.103881802 |
| 716 | G | C | 0.258579754 | 0.205579693 | 302 | I | S | 0.2552956 | 0.169641843 |
| 185 | L | M | 0.258521471 | 0.171738368 | 866 | S | I | 0.255156321 | 0.209048192 |
| 407 | K | N | 0.258498581 | 0.130697064 | 472 | K | M | 0.255025429 | 0.186702335 |
| 973 | W | C | 0.258383156 | 0.162271324 | 165 | R | S | 0.25497678 | 0.100932181 |
| 419 | E | [stop] | 0.258326013 | 0.179526252 | 242 | K | R | 0.254948866 | 0.230748057 |
| 457 | R | K | 0.258323684 | 0.189885325 | 311 | --- | KLK | 0.25494628 | 0.09906032 |
| 876 | S | R | 0.258284608 | 0.118534232 | 200 | V | E | 0.254874846 | 0.123567532 |
| 19 | T | S | 0.258270715 | 0.163493921 | 129 | C | R | 0.25474894 | 0.168215252 |
| 680 | F | S | 0.258237866 | 0.129529513 | 284 | P | A | 0.254723328 | 0.141080203 |
| 2 | E | A | 0.257800465 | 0.161538463 | 232 | --- | CMG | 0.254645266 | 0.200305653 |
| 20 | K | D | 0.257606921 | 0.080857215 | 946 | N | S | 0.2545847 | 0.199844301 |
| 481 | K | E | 0.257527339 | 0.131433394 | 80 | I | V | 0.254434146 | 0.224490053 |
| 227 | A | P | 0.257425537 | 0.162403215 | 327 | G | V | 0.25442364 | 0.168129037 |
| 319 | A | G | 0.25734846 | 0.183688663 | 107 | I | V | 0.254364427 | 0.144921072 |
| 773 | R | T | 0.257312824 | 0.076585471 | 777 | R | I | 0.254281708 | 0.219559132 |
| 59 | S | R | 0.257311236 | 0.098683009 | 801 | L | P | 0.254280774 | 0.139428109 |
| 522 | G | D | 0.257141461 | 0.205906219 | 417 | Y | H | 0.254230823 | 0.102936144 |
| 164 | E | D | 0.257089377 | 0.152824439 | 251 | Q | L | 0.254085129 | 0.154282551 |
| 705 | QA | R- | 0.257083631 | 0.186668119 | 856 | Y | [stop] | 0.254033585 | 0.087466157 |
| 82 | H | Y | 0.256846745 | 0.145259346 | 753 | I | F | 0.25397349 | 0.160875608 |
| 606 | G | R | 0.256772211 | 0.222683526 | 303 | W | G | 0.253842324 | 0.162875151 |
| 281 | P | L | 0.256724807 | 0.103452649 | 852 | Y | H | 0.253666441 | 0.130229811 |
| 471 | D | Y | 0.256649107 | 0.251689277 | 223 | P | S | 0.253640033 | 0.10193396 |
| 231 | A | S | 0.256583564 | 0.187236499 | 472 | K | [stop] | 0.253606489 | 0.18360472 |
| 433 | K | N | 0.256518065 | 0.138408672 | 471 | D | N | 0.250823008 | 0.230246417 |
| 883 | S | G | 0.256375244 | 0.115658726 | 714 | R | [stop] | 0.250772621 | 0.098784657 |
| 672 | P | A | 0.256302042 | 0.169194225 | 192 | A | S | 0.25063862 | 0.18266448 |
| 681 | KD | R- | 0.256180855 | 0.206050883 | 668 | A | D | 0.250605134 | 0.186660163 |
| 762 | G | A | 0.256159485 | 0.149790153 | 147 | -- | KG | 0.250457437 | 0.166419391 |
| 774 | Q | R | 0.256113556 | 0.176872341 | 464 | IE | DR | 0.250457437 | 0.129773988 |
| 630 | P | T | 0.255980317 | 0.147464802 | 325 | -- | LK | 0.250457437 | 0.197198993 |
| 151 | H | Q | 0.255948941 | 0.118092357 | 812 | C | R | 0.250440238 | 0.175896886 |
| 38 | PDL | LT[stop] | 0.255810824 | 0.132108929 | 215 | G | C | 0.250425413 | 0.161826099 |
| 240 | LT | PV | 0.255810824 | 0.138991378 | 564 | G | D | 0.250350924 | 0.110254953 |
| 851 | T | S | 0.25343316 | 0.097399235 | 787 | A | D | 0.250325364 | 0.160958271 |
| 725 | K | E | 0.253359857 | 0.175271591 | 674 | G | V | 0.25029228 | 0.086627759 |
| 115 | V | L | 0.253354021 | 0.093695173 | 182 | T | A | 0.250160953 | 0.131790182 |
| 918 | T | I | 0.253156435 | 0.23080792 | 383 | S | R | 0.250148943 | 0.108851149 |
| 630 | P | L | 0.252953716 | 0.223745102 | 497 | E | G | 0.250036476 | 0.073841396 |
| 75 | E | Q | 0.252809731 | 0.120415311 | 154 | Y | C | 0.250036476 | 0.229055007 |
| 480 | L | M | 0.252718021 | 0.192126204 | 827 | K | R | 0.250016633 | 0.209047833 |
| 197 | S | T | 0.252713621 | 0.125864993 | 722 | Y | [stop] | 0.249927847 | 0.149439604 |
| 779 | E | Q | 0.25259488 | 0.11277405 | 380 | Y | H | 0.249902562 | 0.080398395 |
| 340 | EV | DC | 0.252472535 | 0.047624791 | 68 | K | [stop] | 0.249695921 | 0.134323821 |
| 12 | R | K | 0.252469729 | 0.189301078 | 178 | D | Y | 0.24960373 | 0.233005696 |
| 515 | A | S | 0.252433747 | 0.168422609 | 880 | D | V | 0.249521617 | 0.133706258 |
| 615 | ---- | VIEK (SEQ | 0.252369421 | 0.112001396 | 543 | K | R | 0.249512007 | 0.164262829 |
| | | ID NO: 3778) | | | | | | | |
| 513 | N | S | 0.252353713 | 0.094778563 | 101 | Q | E | 0.249509933 | 0.220597507 |
| 274 | A | P | 0.252335379 | 0.222801897 | 261 | L | P | 0.249467079 | 0.135680009 |
| 474 | E | Q | 0.252314637 | 0.161495393 | 410 | G | A | 0.249451996 | 0.157770206 |
| 898 | K | E | 0.252289386 | 0.197783073 | 916 | --------- | FETHAAEQA | 0.249445316 | 0.231377364 |
| | | | | | | | (SEQ | | |
| | | | | | | | ID NO: 3410) | | |
| 397 | Q | K | 0.252164481 | 0.217428232 | 467 | L | M | 0.249366626 | 0.154018589 |
| 455 | W | S | 0.25204917 | 0.248519347 | 745 | A | V | 0.249363082 | 0.18169323 |
| 135 | P | S | 0.252041319 | 0.143618662 | 773 | R | K | 0.249259705 | 0.143796066 |
| 500 | N | D | 0.252036438 | 0.129905572 | 221 | S | Y | 0.249177365 | 0.225580403 |
| 204 | S | I | 0.252028425 | 0.131493678 | 953 | DK | CL | 0.248980289 | 0.153230139 |
| 235 | A | T | 0.251989659 | 0.158776047 | 29 | KT | NC | 0.247444507 | 0.126896702 |
| 839 | I | M | 0.251899392 | 0.164461403 | 777 | R | G | 0.247073817 | 0.140696212 |
| 473 | D | N | 0.251700557 | 0.215226558 | 720 | R | T | 0.246870637 | 0.139065914 |
| 715 | A | D | 0.251688144 | 0.14707302 | 529 | --- | YLI | 0.246804685 | 0.066320143 |
| 352 | K | E | 0.251658395 | 0.165058904 | 977 | V | M | 0.24675063 | 0.232768749 |
| 413 | R | I | 0.251517421 | 0.230382833 | 414 | G | C | 0.246666689 | 0.173156358 |
| 272 | G | R | 0.251488679 | 0.185835986 | 487 | G | D | 0.246317089 | 0.205561043 |
| 647 | S | R | 0.251423405 | 0.100129809 | 696 | S | G | 0.246296346 | 0.111834798 |
| 333 | L | M | 0.251344003 | 0.196286065 | 515 | A | G | 0.246293045 | 0.17108612 |
| 964 | F | Y | 0.25104576 | 0.166483614 | 438 | -- | EE | 0.246243471 | 0.172505379 |
| 474 | E | K | 0.250927827 | 0.172968831 | 730 | A | S | 0.246013083 | 0.141113967 |
| 751 | M | V | 0.250846737 | 0.147715329 | 574 | N | D | 0.245981475 | 0.227302881 |
| 213 | ------ | QIGGNS | 0.248980289 | 0.134226006 | 747 | T | S | 0.245965899 | 0.17316365 |
| | | (SEQ ID NO: | | | | | | | |
| | | 3639) | | | | | | | |
| 57 | P | H | 0.248900571 | 0.215896368 | 740 | D | Y | 0.245945789 | 0.167910919 |
| 301 | V | L | 0.24886944 | 0.106508651 | 640 | R | I | 0.245900817 | 0.188813199 |
| 586 | A | P | 0.248863678 | 0.211216154 | 3 | I | F | 0.245678 | 0.179390362 |
| 909 | F | Y | 0.248749713 | 0.182356511 | 355 | N | D | 0.245670687 | 0.09594124 |
| 626 | R | T | 0.248743703 | 0.208846467 | 371 | Y | [stop] | 0.245500092 | 0.105713424 |
| 186 | G | R | 0.24871786 | 0.199871451 | 51 | P | S | 0.24544462 | 0.203086773 |
| 645 | D | N | 0.248657263 | 0.126033155 | 28 | M | L | 0.245403036 | 0.189135882 |
| 173 | K | R | 0.24855018 | 0.153000538 | 458 | A | D | 0.245377197 | 0.208634207 |
| 519 | Q | [stop] | 0.248535487 | 0.209163595 | 572 | N | I | 0.24524576 | 0.164550203 |
| 888 | R | I | 0.248471987 | 0.104169936 | 959 | E | [stop] | 0.245144817 | 0.219795779 |
| 491 | G | C | 0.248444417 | 0.204717262 | 527 | N | S | 0.245098015 | 0.16437657 |
| 527 | N | K | 0.248397784 | 0.121054149 | 321 | P | S | 0.245086017 | 0.160736605 |
| 893 | L | V | 0.248370955 | 0.162725859 | 579 | N | K | 0.244981546 | 0.165374413 |
| 379 | P | H | 0.248321642 | 0.237522233 | 707 | A | P | 0.244857358 | 0.22019856 |
| 900 | F | L | 0.248316685 | 0.187112489 | 414 | G | A | 0.244717702 | 0.113316145 |
| 974 | ----- | KPAV (SEQ | 0.24830974 | 0.09950399 | 963 | S | G | 0.244450471 | 0.188301401 |
| | | ID NO: | | | | | | | |
| | | 3518)[stop] | | | | | | | |
| 409 | H | R | 0.248289463 | 0.198716638 | 108 | D | H | 0.244382837 | 0.099322593 |
| 278 | I | T | 0.248133293 | 0.145997719 | 19 | T | R | 0.244301214 | 0.22638105 |
| 230 | ----- | DACMG | 0.248087937 | 0.141736439 | 457 | R | S | 0.244059876 | 0.203207391 |
| | | (SEQ ID NO: | | | | | | | |
| | | 3342) | | | | | | | |
| 412 | ------ | DWGKVY | 0.248000785 | 0.085936492 | 735 | R | Q | 0.243928198 | 0.170841115 |
| | | (SEQ ID NO: | | | | | | | |
| | | 3370) | | | | | | | |
| 548 | E | V | 0.244464905 | 0.11615159 | 280 | L | P | 0.243719915 | 0.122012762 |
| 135 | P | H | 0.247697198 | 0.24068468 | 529 | Y | C | 0.241113191 | 0.148105236 |
| 824 | V | E | 0.247676063 | 0.211426874 | 102 | P | S | 0.241100901 | 0.126616893 |
| 250 | H | N | 0.247644364 | 0.173527273 | 568 | P | R | 0.241086845 | 0.174639843 |
| 101 | Q | [stop] | 0.247598429 | 0.141658982 | 416 | V | L | 0.24098406 | 0.086334529 |
| 364 | F | S | 0.247520151 | 0.139448351 | 834 | G | S | 0.240965197 | 0.161966438 |
| 420 | A | G | 0.247498728 | 0.234162787 | 322 | L | M | 0.240965197 | 0.161073617 |
| 627 | Q | P | 0.243601279 | 0.172067752 | 538 | G | s | 0.240933783 | 0.072861862 |
| 571 | -- | VN | 0.243561744 | 0.078796567 | 536 | K | E | 0.240888218 | 0.130971778 |
| 25 | T | A | 0.243399906 | 0.118102255 | 676 | P | s | 0.240757682 | 0.111329254 |
| 129 | C | S | 0.243399597 | 0.045331126 | 108 | D | E | 0.240718917 | 0.12602791 |
| 522 | G | S | 0.243323907 | 0.089702225 | 217 | N | K | 0.240713475 | 0.15867648 |
| 695 | E | K | 0.243320032 | 0.148139423 | 342 | D | E | 0.24062135 | 0.069616641 |
| 603 | L | V | 0.243217969 | 0.148743728 | 471 | D | H | 0.240564636 | 0.181535186 |
| 404 | H | Q | 0.242964457 | 0.173626579 | 218 | S | N | 0.240529528 | 0.151826239 |
| 469 | E | Q | 0.242802772 | 0.126770274 | 191 | R | I | 0.240513696 | 0.229207246 |
| 484 | KWY | NSS | 0.242735572 | 0.182387025 | 963 | --- | SFY | 0.240421887 | 0.098315268 |
| 797 | L | V | 0.2425558 | 0.204091719 | 77 | K | N | 0.240381155 | 0.116252284 |
| 928 | I | F | 0.242416049 | 0.232458614 | 637 | ---- | TFER (SEQ | 0.240288787 | 0.148900082 |
| | | | | | | | ID NO: 3744) | | |
| 974 | K | R | 0.242320513 | 0.114367362 | 571 | V | L | 0.240279118 | 0.074639743 |
| 687 | P | L | 0.242304633 | 0.20007901 | 346 | M | T | 0.240147015 | 0.108146398 |
| 885 | T | R | 0.242245862 | 0.204992576 | 512 | Y | [stop] | 0.240104852 | 0.068415116 |
| 768 | T | S | 0.242193729 | 0.178836886 | 430 | G | C | 0.240047705 | 0.20806366 |
| 588 | ---- | GKRQ (SEQ | 0.242084293 | 0.124769338 | 599 | D | G | 0.239869359 | 0.206138755 |
| | | ID NO: 3440) | | | | | | | |
| 262 | ------ | ANLKD1 | 0.242084293 | 0.137081914 | 462 | F | s | 0.23971457 | 0.144092402 |
| | | (SEQ ID NO: | | | | | | | |
| | | 3325) | | | | | | | |
| 246 | I | C | 0.242084293 | 0.107590717 | 724 | S | R | 0.239681347 | 0.127922837 |
| 288 | E | [stop] | 0.242056668 | 0.219648186 | 61 | T | S | 0.239626948 | 0.164373644 |
| 978 | -[stop] | YV | 0.242009218 | 0.097706533 | 525 | K | [stop] | 0.239380142 | 0.131802154 |
| 110 | R | [stop] | 0.241965346 | 0.120709959 | 296 | V | E | 0.239355864 | 0.120748179 |
| 741 | L | M | 0.241912289 | 0.193137515 | 968 | K | Q | 0.238999998 | 0.129755167 |
| 72 | D | Y | 0.241758248 | 0.224435844 | 617 | E | K | 0.238964823 | 0.084548152 |
| 653 | N | Y | 0.24166971 | 0.0887834 | 120 | E | K | 0.238945442 | 0.100801456 |
| 324 | R | [stop] | 0.241651421 | 0.106997792 | 44 | L | V | 0.238860984 | 0.10949901 |
| 293 | Y | D | 0.241440886 | 0.202068751 | 315 | G | R | 0.238751925 | 0.215543005 |
| 695 | E | A | 0.241330438 | 0.115436697 | 87 | E | [stop] | 0.238731064 | 0.177299521 |
| 798 | -------- | SKTLAQYT | 0.241309883 | 0.196326087 | 204 | S | C | 0.236855446 | 0.164372504 |
| | | (SEQ ID NO: | | | | | | | |
| | | 3714) | | | | | | | |
| 866 | S | G | 0.241237257 | 0.109329768 | 82 | H | Q | 0.236837713 | 0.172606609 |
| 818 | S | G | 0.238509249 | 0.201919192 | 861 | ------- | VVKDLSVE | 0.236770505 | 0.195127344 |
| | | | | | | | (SEQ ID NO: | | |
| | | | | | | | 3837) | | |
| 189 | G | V | 0.238447609 | 0.179422249 | 493 | P | L | 0.236700832 | 0.181806123 |
| 394 | A | D | 0.238439863 | 0.125867824 | 474 | E | G | 0.236695789 | 0.180206764 |
| 861 | - | V | 0.238439176 | 0.202222792 | 302 | I | F | 0.236588615 | 0.136160472 |
| 357 | K | E | 0.238434177 | 0.184905545 | 109 | Q | R | 0.236576305 | 0.166840659 |
| 353 | L | V | 0.23831895 | 0.17206072 | 97 | S | R | 0.236508024 | 0.179878709 |
| 488 | D | V | 0.2382354 | 0.188903119 | 40 | L | V | 0.236210141 | 0.21459356 |
| 684 | ----- | LGNPT (SEQ | 0.2382268 | 0.157487774 | 761 | F | C | 0.236145536 | 0.170046245 |
| | | ID NO: 3549) | | | | | | | |
| 376 | A | V | 0.238191318 | 0.142572457 | 50 | K | N | 0.236137845 | 0.22219675 |
| 349 | N | D | 0.238174065 | 0.053089179 | 205 | N | K | 0.236073257 | 0.12180008 |
| 331 | F | S | 0.238131141 | 0.093269792 | 399 | G | D | 0.236045787 | 0.181873656 |
| 971 | E | D | 0.238076025 | 0.194709418 | 521 | D | Y | 0.235934057 | 0.180076567 |
| 775 | Y | F | 0.238057448 | 0.214475137 | 665 | A | D | 0.235822456 | 0.220273467 |
| 730 | A | T | 0.238038323 | 0.175731569 | 252 | K | R | 0.235675801 | 0.120466673 |
| 631 | --- | ALF | 0.237949975 | 0.190053084 | 646 | S | R | 0.235675637 | 0.183914638 |
| 504 | D | H | 0.23794567 | 0.139048842 | 102 | P | A | 0.235653058 | 0.16760539 |
| 94 | G | D | 0.237937578 | 0.15570335 | 810 | S | N | 0.235539825 | 0.164257896 |
| 291 | E | [stop] | 0.237828954 | 0.19900832 | 936 | R | S | 0.235496123 | 0.188093786 |
| 871 | R | I | 0.237759309 | 0.236033629 | 111 | K | R | 0.235492778 | 0.118354865 |
| 761 | F | Y | 0.237669703 | 0.128380283 | 220 | A | V | 0.235467868 | 0.198253635 |
| 910 | ---- | VCLN (SEQ | 0.237633429 | 0.152561858 | 855 | --- | RYK | 0.235222552 | 0.156668306 |
| | | ID NO: 3768) | | | | | | | |
| 731 | D | Y | 0.237566392 | 0.167223625 | 354 | I | N | 0.235178848 | 0.098023234 |
| 245 | D | A | 0.237553897 | 0.189220496 | 158 | C | F | 0.235135625 | 0.169427052 |
| 979 | L-E | VWS | 0.237546222 | 0.150693183 | 689 | H | R | 0.235102048 | 0.220671524 |
| 208 | V | E | 0.237546113 | 0.17752812 | 594 | E--F | GRII (SEQ ID | 0.235051862 | 0.132444365 |
| | | | | | | | NO: 3451) | | |
| 483 | Q | R | 0.23746372 | 0.159123209 | 154 | Y | D | 0.234980588 | 0.232501764 |
| 634 | V | M | 0.237398857 | 0.152995502 | 870 | D | V | 0.234951394 | 0.118777361 |
| 837 | T | I | 0.237183554 | 0.104666535 | 198 | I | N | 0.234906329 | 0.184047389 |
| 479 | E | Q | 0.237085358 | 0.157162064 | 76 | M | I | 0.234796263 | 0.126238567 |
| 555 | F | V | 0.237065318 | 0.182110462 | 434 | H | N | 0.234726089 | 0.143174214 |
| 872 | LS | PV | 0.23698628 | 0.179042308 | 570 | E | Q | 0.232497705 | 0.099759258 |
| 601 | L | P | 0.236954247 | 0.122470012 | 645 | D | E | 0.2323596 | 0.127143455 |
| 127 | F | L | 0.236892252 | 0.129435749 | 54 | I | N | 0.23228755 | 0.182788712 |
| 484 | --KW | NSSL (SEQ | 0.234680329 | 0.165662856 | 725 | K | R | 0.232253631 | 0.11253677 |
| | | ID NO: 3599) | | | | | | | |
| 49 | K | [stop] | 0.234415257 | 0.114263318 | 771 | A | S | 0.232158252 | 0.16845905 |
| 896 | L | P | 0.234287413 | 0.192149813 | 896 | L | V | 0.232108864 | 0.141878039 |
| 530 | L | V | 0.234192802 | 0.173965176 | 487 | G | V | 0.232053935 | 0.22651513 |
| 643 | V | A | 0.234106948 | 0.176627185 | 655 | I | V | 0.231994505 | 0.148078533 |
| 711 | E | K | 0.234002178 | 0.154011045 | 708 | K | R | 0.231988811 | 0.183732743 |
| 918 | ----- | THAAEQ | 0.23373891 | 0.117744474 | 699 | E | D | 0.231934703 | 0.178386576 |
| | | (SEQ ID NO: | | | | | | | |
| | | 3747) | | | | | | | |
| 473 | D | E | 0.233630727 | 0.181285916 | 446 | A | P | 0.231896096 | 0.131534649 |
| 666 | V | E | 0.233615017 | 0.210063502 | 902 | H | P | 0.231793863 | 0.226418313 |
| 610 | ------- | LANGRVIE | 0.233598549 | 0.098900798 | 555 | F | S | 0.231772683 | 0.154329003 |
| | | (SEQ ID NO: | | | | | | | |
| | | 3538) | | | | | | | |
| 463 | V | A | 0.233582437 | 0.13705941 | 685 | G | R | 0.231646911 | 0.113490558 |
| 771 | A | V | 0.233335501 | 0.144017771 | 430 | G | A | 0.231581897 | 0.168869877 |
| 89 | Q | H | 0.233314663 | 0.120225936 | 423 | R | G | 0.231294589 | 0.188648387 |
| 18 | N | D | 0.233234266 | 0.100130745 | 773 | R | S | 0.231238362 | 0.139470334 |
| 547 | P | A | 0.233232691 | 0.192665943 | 148 | --- | GKP | 0.231166477 | 0.084708483 |
| 628 | D | H | 0.233191566 | 0.113338873 | 795 | TY | PG | 0.231166477 | 0.229360354 |
| 290 | I | V | 0.233178351 | 0.147527858 | 598 | N | S | 0.230890539 | 0.114382772 |
| 837 | ---- | TTIN (SEQ ID | 0.233038063 | 0.141130326 | 109 | Q | [stop] | 0.230738213 | 0.089332392 |
| | | NO: 3761) | | | | | | | |
| 909 | -- | FV | 0.233038063 | 0.131142006 | 481 | ---- | KLQK (SEQ | 0.23071553 | 0.20441951 |
| | | | | | | | ID NO: 3513) | | |
| 260 | R | G | 0.232970656 | 0.120191772 | 592 | -GR | DNQ | 0.230655892 | 0.071944702 |
| 707 | ------- | AKEVEQR | 0.232896265 | 0.116012039 | 254 | I | T | 0.2306357 | 0.069580284 |
| | | (SEQ ID NO: | | | | | | | |
| | | 3314) | | | | | | | |
| 638 | F | S | 0.232893598 | 0.149395863 | 530 | L | R | 0.230571343 | 0.193066361 |
| 671 | D | A | 0.232880356 | 0.163658679 | 365 | W | [stop] | 0.230333383 | 0.12753339 |
| 443 | S | T | 0.232784832 | 0.170920909 | 131 | Q | R | 0.2302555 | 0.206903114 |
| 392 | K | N | 0.232687633 | 0.108105318 | 244 | Q | E | 0.230190451 | 0.222512927 |
| 500 | N | I | 0.232640715 | 0.1305158 | 900 | F | I | 0.230181139 | 0.149890666 |
| 111 | K | E | 0.232613623 | 0.097737029 | 318 | E | Q | 0.230160478 | 0.212890421 |
| 610 | L | V | 0.229644521 | 0.180175813 | 312 | L | M | 0.230110955 | 0.204915228 |
| 847 | E | G | 0.229640073 | 0.111868196 | 106 | N | S | 0.230101564 | 0.155287559 |
| 636 | -- | LT | 0.229485665 | 0.192188426 | 968 | K | R | 0.230017803 | 0.168949701 |
| 665 | A | G | 0.229408129 | 0.212381399 | 631 | A | P | 0.229723383 | 0.159718894 |
| 82 | H | R | 0.229295108 | 0.108155794 | 864 | D | G | 0.226094276 | 0.177950676 |
| 371 | Y | D | 0.229277426 | 0.117283148 | 140 | K | R | 0.226067524 | 0.114127554 |
| 148 | G | V | 0.229238098 | 0.159823444 | 814 | F | S | 0.225959256 | 0.114511043 |
| 443 | S | I | 0.229142738 | 0.169822985 | 215 | G | D | 0.225350951 | 0.086324983 |
| 660 | G | C | 0.229029418 | 0.194710612 | 138 | V | L | 0.225143743 | 0.155359682 |
| 181 | V | D | 0.228966959 | 0.164951106 | 192 | A | T | 0.22512485 | 0.144695235 |
| 832 | A | P | 0.228767879 | 0.092204547 | 502 | I | S | 0.225038868 | 0.197567126 |
| 152 | T | A | 0.228705386 | 0.182569685 | 494 | F | V | 0.224968248 | 0.143764694 |
| 685 | G | A | 0.228675631 | 0.17392363 | 162 | E | D | 0.224950043 | 0.153078143 |
| 112 | L | P | 0.22866263 | 0.221195984 | 788 | Y | [stop] | 0.22492674 | 0.129943744 |
| 214 | I | T | 0.22857342 | 0.11423526 | 263 | N | I | 0.224722541 | 0.117014395 |
| 610 | L | M | 0.22841473 | 0.205382368 | 918 | ------- | THAAEQA | 0.224719714 | 0.202778103 |
| | | | | | | | (SEQ ID NO: | | |
| | | | | | | | 3748) | | |
| 110 | R | G | 0.228257249 | 0.086720324 | 272 | G | A | 0.224696933 | 0.211543463 |
| 590 | R | S | 0.228041456 | 0.143022556 | 322 | L | V | 0.2246772 | 0.156881144 |
| 596 | I | M | 0.227907909 | 0.117874099 | 132 | C | R | 0.224659007 | 0.146010501 |
| 1 | Q | P | 0.227785203 | 0.168369144 | 657 | I | F | 0.224649177 | 0.161870244 |
| 567 | V | E | 0.227660557 | 0.156302233 | 917 | - | E | 0.224592553 | 0.150266826 |
| 32 | L | V | 0.227635279 | 0.12966479 | 704 | ------ | IQAAKE | 0.224567514 | 0.109443666 |
| | | | | | | | (SEQ ID NO: | | |
| | | | | | | | 3481) | | |
| 65 | N | S | 0.22749218 | 0.063907676 | 328 | --- | FPS | 0.224567514 | 0.088644166 |
| 291 | E | G | 0.227296993 | 0.128103388 | 455 | W | R | 0.224240948 | 0.159412878 |
| 635 | A | V | 0.22713711 | 0.159876533 | 528 | -- | LY | 0.224210461 | 0.204469226 |
| 894 | S | I | 0.227093532 | 0.165363718 | 289 | G | A | 0.224158556 | 0.07475664 |
| 675 | C | R | 0.227077437 | 0.19145584 | 477 | RCE | SFS | 0.224109734 | 0.175971589 |
| 863 | K | E | 0.227027728 | 0.176903569 | 290 | I | M | 0.224106784 | 0.121750806 |
| 130 | S | N | 0.226933191 | 0.162445952 | 699 | EK | AV | 0.223971566 | 0.120407858 |
| 187 | K | E | 0.226883263 | 0.185467572 | 190 | ------ | QRALDFY | 0.223971566 | 0.118248938 |
| | | | | | | | (SEQ ID NO: | | |
| | | | | | | | 3646) | | |
| 330 | S | G | 0.226753105 | 0.138020012 | 287 | K | [stop] | 0.223966216 | 0.119362605 |
| 224 | V | A | 0.226536103 | 0.153342124 | 33 | V | A | 0.223884337 | 0.200194354 |
| 802 | A | T | 0.226368502 | 0.154358709 | 321 | P | R | 0.223833871 | 0.153353055 |
| 148 | G | S | 0.226168476 | 0.097680006 | 149 | K | [stop] | 0.221989288 | 0.160692576 |
| 732 | D | E | 0.226134547 | 0.109002487 | 230 | --- | DAC | 0.221929991 | 0.119956442 |
| 350 | V | L | 0.223803585 | 0.123552417 | 559 | -I | TV | 0.221929991 | 0.162385076 |
| 598 | N | D | 0.223755594 | 0.127015451 | 125 | S | T | 0.221924231 | 0.192354491 |
| 784 | A | V | 0.22374846 | 0.140061096 | 738 | A | P | 0.221764129 | 0.166374434 |
| 540 | L | P | 0.223660834 | 0.130300184 | 389 | K | L | 0.221512528 | 0.096823472 |
| 330 | S | R | 0.2236138 | 0.142019721 | 829 | K | M | 0.22130603 | 0.111760034 |
| 162 | E | Q | 0.223613045 | 0.201165398 | 435 | I | V | 0.221227154 | 0.143247597 |
| 128 | A | V | 0.223401934 | 0.126557909 | 626 | R | S | 0.221038435 | 0.198631408 |
| 296 | V | L | 0.223401818 | 0.13392173 | 135 | P | R | 0.221017429 | 0.116069626 |
| 634 | V | E | 0.223309652 | 0.118175475 | 203 | E | Q | 0.22076143 | 0.119826394 |
| 356 | E | Q | 0.22323735 | 0.143945409 | 783 | T | I | 0.220740744 | 0.134860122 |
| 289 | G | V | 0.223202197 | 0.145913012 | 672 | P | S | 0.220729114 | 0.141569742 |
| 805 | T | N | 0.223188037 | 0.139245678 | 361 | G | D | 0.220639166 | 0.141910298 |
| 599 | D | Y | 0.223008187 | 0.183323322 | 690 | I | M | 0.220631897 | 0.180897111 |
| 246 | I | M | 0.222998811 | 0.092368092 | 552 | A | G | 0.220614882 | 0.110523427 |
| 36 | M | K | 0.222893666 | 0.113406903 | 441 | R | I | 0.220543521 | 0.155159451 |
| 476 | C | [stop] | 0.222743024 | 0.176188321 | 218 | S | R | 0.220420945 | 0.153071466 |
| 464 | I | V | 0.222701858 | 0.18421718 | 917 | ------ | ETHAAE | 0.220288736 | 0.09840913 |
| | | | | | | | (SEQ ID NO: | | |
| | | | | | | | 3400) | | |
| 224 | V | L | 0.222626458 | 0.136476862 | 204 | S | R | 0.220214876 | 0.101819626 |
| 42 | E | G | 0.22255062 | 0.189996134 | 255 | K | E | 0.220080844 | 0.12573371 |
| 832 | A | S | 0.222538216 | 0.190249328 | 479 | E | D | 0.220079089 | 0.099777598 |
| 734 | V | I | 0.222476682 | 0.141366416 | 438 | E | G | 0.219979549 | 0.120742867 |
| 146 | D | H | 0.22246095 | 0.16577062 | 605 | T | 1 | 0.219976898 | 0.126979027 |
| 755 | AN | DS | 0.222404547 | 0.10970681 | 109 | Q | E | 0.219959218 | 0.140761458 |
| 581 | I | V | 0.222357666 | 0.17105795 | 744 | Y | C | 0.219956045 | 0.132833086 |
| 698 | K | [stop] | 0.222296953 | 0.103211977 | 930 | ------ | RSWLFL | 0.219822658 | 0.120132898 |
| | | | | | | | (SEQ ID NO: | | |
| | | | | | | | 3689) | | |
| 507 | G | D | 0.22225927 | 0.153400026 | 172 | H | Q | 0.219757029 | 0.10461302 |
| 246 | I | V | 0.222098073 | 0.120973819 | 329 | P | A | 0.219753668 | 0.110968401 |
| 47 | L | P | 0.222066189 | 0.162841956 | 783 | T | S | 0.219504994 | 0.118049041 |
| 301 | VI | CL | 0.222059585 | 0.122617461 | 610 | L | P | 0.219499239 | 0.160199117 |
| 210 | PL | DR | 0.222059585 | 0.108090576 | 433 | --- | KHI | 0.216309574 | 0.092546366 |
| 174 | ------ | PEANDE | 0.222059585 | 0.182232379 | 375 | E | [stop] | 0.216261145 | 0.199757211 |
| | | (SEQ ID NO: | | | | | | | |
| | | 3616) | | | | | | | |
| 160 | --- | VSE | 0.222059585 | 0.137662445 | 297 | V | A | 0 216143366 | 0.15509483 |
| 68 | K | E | 0.222044865 | 0.16348242 | 148 | ------- | GKPHTNYF | 0.216132461 | 0.211503255 |
| | | | | | | | (SEQ ID NO: | | |
| | | | | | | | 3439) | | |
| 38 | P | A | 0.219404694 | 0.107368636 | 645 | D | V | 0.21604012 | 0.117781298 |
| 446 | A | V | 0.218887024 | 0.176662627 | 147 | KG | R- | 0.215998635 | 0.103939398 |
| 41 | R | K | 0.218858764 | 0.128896181 | 292 | A | S | 0.215943856 | 0.157240024 |
| 810 | S | R | 0.21870856 | 0.129689435 | 387 | R | G | 0.215798372 | 0.151215331 |
| 83 | V | L | 0.218625171 | 0.138945755 | 157 | R | T | 0.215790548 | 0.152247144 |
| 474 | E | D | 0.218570822 | 0.130400355 | 203 | E | K | 0.215703649 | 0.168783031 |
| 712 | Q | [stop] | 0.218254094 | 0.091444311 | 123 | T | S | 0.21570133 | 0.105624839 |
| 371 | Y | H | 0.218137961 | 0.189187449 | 383 | S | G | 0.215603433 | 0.137401501 |
| 35 | V | L | 0.218110612 | 0.095949997 | 310 | Q | [stop] | 0.21551735 | 0.135329921 |
| 687 | P | R | 0.21806458 | 0.159278352 | 592 | G | A | 0.215456343 | 0.13373272 |
| 621 | Y | N | 0.218036238 | 0.089590425 | 562 | K | R | 0.215325036 | 0.122831356 |
| 753 | I | N | 0.21792347 | 0.101271232 | 951 | N | S | 0.21531813 | 0.214926405 |
| 337 | Q | L | 0.217694196 | 0.180223104 | 823 | R | I | 0.215273573 | 0.191310901 |
| 366 | Q | E | 0.217564323 | 0.195945495 | 723 | A | P | 0.215193332 | 0.108699964 |
| 156 | G | R | 0.217510036 | 0.186872459 | 713 | R | T | 0.215008884 | 0.104394548 |
| 813 | G | A | 0.217404463 | 0.109971024 | 878 | N | I | 0.214931515 | 0.11752804 |
| 911 | C | W | 0.217360044 | 0.181625646 | 145 | N | H | 0.214892161 | 0.185408691 |
| 896 | L | Q | 0.217312492 | 0.09770592 | 338 | A | T | 0.21480521 | 0.15310635 |
| 395 | R | S | 0.217267056 | 0.103436045 | 169 | L | V | 0.214751891 | 0.163877193 |
| 506 | S | R | 0.217238346 | 0.104753923 | 30 | T | P | 0.214714414 | 0.144104489 |
| 459 | KA | NR | 0.217171538 | 0.126085081 | 164 | E | A | 0.214693055 | 0.151750991 |
| 605 | T | S | 0.217140582 | 0.104288213 | 734 | V | F | 0.214507965 | 0.184315198 |
| 147 | K | R | 0.217113942 | 0.165662771 | 841 | G | V | 0.21449654 | 0.163419397 |
| 358 | K | R | 0.217018444 | 0.148484962 | 848 | G | D | 0.214491489 | 0.166744246 |
| 710 | V | E | 0.216906218 | 0.158321415 | 93 | VGL | WA [stop] | 0.21434042 | 0.171347302 |
| 948 | T | N | 0.216794988 | 0.204294035 | 747 | T | K | 0.214238165 | 0.122971462 |
| 62 | S | T | 0.216604466 | 0.167204921 | 688 | T | K | 0.214222271 | 0.126368648 |
| 827 | K | E | 0.216603742 | 0.107241416 | 878 | N | Y | 0.214205323 | 0.111547616 |
| 457 | R | G | 0.216513116 | 0.052626339 | 190 | Q | E | 0.214170887 | 0.122424442 |
| 159 | N | K | 0.216507269 | 0.109954763 | 901 | ------ | SHRPVQE | 0.212684828 | 0.084903934 |
| | | | | | | | (SEQ ID NO: | | |
| | | | | | | | 3707) | | |
| 177 | N | D | 0.216431319 | 0.179290406 | 459 | K | E | 0.212680715 | 0.093525423 |
| 921 | ------- | AEQAALN | 0.216389396 | 0.149922966 | 228 | L | V | 0.212591965 | 0.092947468 |
| | | (SEQ ID NO: | | | | | | | |
| | | 3308) | | | | | | | |
| 633 | -- | FV | 0.216309574 | 0.179645361 | 831 | T | I | 0.212576099 | 0.16705965 |
| 523 | | VKKLN (SEQ | 0.214126014 | 0.14801882 | 819 | A | T | 0.212522918 | 0.164976137 |
| | | ID NO: 3782) | | | | | | | |
| 792 | --- | PSK | 0.214126014 | 0.088425611 | 645 | D | G | 0.21251225 | 0.121902674 |
| 171 | --- | PHK | 0.214126014 | 0.186440571 | 794 | K | R | 0.212502396 | 0.178916123 |
| 918 | -- | TH | 0.214126014 | 0.10224323 | 859 | Q | P | 0.212311083 | 0.170329714 |
| 833 | T | S | 0.214086868 | 0.0993742 | 738 | A | G | 0.212248976 | 0.161293316 |
| 72 | D | E | 0.214062412 | 0.115630034 | 409 | H | Q | 0.212187222 | 0.201696134 |
| 560 | N | K | 0.213945541 | 0.173784949 | 192 | ----- | ALDFY (SEQ | 0.212165997 | 0.132724298 |
| | | | | | | | ID NO: 3317) | | |
| 906 | Q | L | 0.213845132 | 0.187470303 | 782 | ------ | LTAKLA | 0.212165997 | 0.121732843 |
| | | | | | | | (SEQ ID NO: | | |
| | | | | | | | 3580) | | |
| 461 | S | I | 0.21384342 | 0.180386801 | 86 | EEF | DCL | 0.212165997 | 0.090389548 |
| 622 | N | I | 0.213809938 | 0.161761781 | 251 | Q | H | 0.212109948 | 0.151365816 |
| 768 | T | I | 0.213809607 | 0.08102538 | 197 | S | R | 0.211641987 | 0.087103971 |
| 204 | --- | SNH | 0.21345676 | 0.114570097 | 196 | Y | C | 0.211596178 | 0.195825393 |
| 944 | - | Q | 0.213449244 | 0.157411492 | 125 | S | I | 0.211507893 | 0.117116373 |
| 49 | K | R | 0.213334728 | 0.181645679 | 237 | A | T | 0.211485023 | 0.118730598 |
| 411 | E | [stop] | 0.213222053 | 0.149931485 | 574 | N | S | 0.211257767 | 0.135650502 |
| 719 | S | A | 0.213134782 | 0.140566151 | 73 | Y | C | 0.211200986 | 0.169366394 |
| 731 | D | E | 0.213022905 | 0.120709041 | 380 | Y | [stop] | 0.21093329 | 0.132735624 |
| 475 | F | S | 0.213010505 | 0.137035236 | 219 | C | Y | 0.210905605 | 0.190298454 |
| 305 | N | K | 0.213008678 | 0.108878566 | 777 | R | S | 0.210879382 | 0.15535129 |
| 30 | TL | PC | 0.212945774 | 0.075648365 | 799 | ------ | KTLAQYT | 0.210719207 | 0.130227708 |
| | | | | | | | (SEQ ID NO: | | |
| | | | | | | | 3530) | | |
| 611 | A | G | 0.212935031 | 0.195766935 | 79 | A | T | 0.210637972 | 0.047863719 |
| 266 | DI | AV | 0.212926287 | 0.127744646 | 654 | L | R | 0.210450467 | 0.143325776 |
| 730 | ---- | ADDM (SEQ | 0.212926287 | 0.097551919 | 479 | E | K | 0.210277517 | 0.147945245 |
| | | ID NO: 3302) | | | | | | | |
| 684 | -- | LG | 0.212926287 | 0.093015719 | 595 | F | I | 0.208631842 | 0.129889087 |
| 979 | LE[stop]GSPG | VSSKDLK | 0.212926287 | 0.091900005 | 765 | G | R | 0.208575469 | 0.10091353 |
| | (SEQ ID NO: | (SEQ ID NO: | | | | | | | |
| | 3251) | 3808) | | | | | | | |
| 241 | ---- | TKYQ (SEQ | 0.212926287 | 0.1464038 | 506 | S | G | 0.208540925 | 0.155512988 |
| | | ID NO: 3751) | | | | | | | |
| 949 | T | I | 0.212862846 | 0.194719268 | 408 | K | R | 0.208534867 | 0.133392724 |
| 709 | E | G | 0.212846074 | 0.116849712 | 171 | P | A | 0.208511912 | 0.145333852 |
| 926 | -- | LN | 0.212734596 | 0.151263965 | 953 | -- | DK | 0.208375969 | 0.185478366 |
| 587 | F | E | 0.210211385 | 0.204490333 | 518 | W | C | 0.208374964 | 0.121746678 |
| 444 | E | Q | 0.210197326 | 0.171958409 | 34 | R | G | 0.208371871 | 0.100655798 |
| 546 | K | Q | 0.210196739 | 0.176398222 | 663 | ---- | IPAV (SEQ ID | 0.208314284 | 0.125213293 |
| | | | | | | | NO: 3479) | | |
| 645 | D | Y | 0.210085231 | 0.190055155 | 737 | T | S | 0.208225559 | 0.129504354 |
| 67 | N | S | 0.210019556 | 0.13100266 | 6 | I | N | 0.208110644 | 0.078448603 |
| 403 | L | P | 0.209919624 | 0.075615563 | 677 | L | M | 0.208075234 | 0.142372791 |
| 452 | L | P | 0.209882094 | 0.127675947 | 456 | L | Q | 0.208040599 | 0.142959764 |
| 733 | M | V | 0.209851123 | 0.136163056 | 190 | Q | R | 0.207948331 | 0.189816674 |
| 872 | L | P | 0.209831548 | 0.152338232 | 382 | S | G | 0.207889255 | 0.137324724 |
| 882 | S | R | 0.209789855 | 0.108285285 | 953 | D | H | 0.207762178 | 0.180457041 |
| 679 | R | T | 0.209762925 | 0.169692137 | 522 | G | R | 0.207711735 | 0.201735272 |
| 553 | ------- | NRFYTVI | 0.209733011 | 0.13607198 | 655 | I | F | 0.207554053 | 0.114186846 |
| | | (SEQ ID NO: | | | | | | | |
| | | 3596) | | | | | | | |
| 650 | ---- | KPMN (SEQ | 0.209706804 | 0.099600175 | 345 | D | N | 0.207459671 | 0.194429167 |
| | | ID NO: 3523) | | | | | | | |
| 802 | AQ | DR | 0.209706804 | 0.100831295 | 619 | T | A | 0.20742287 | 0.107807162 |
| 415 | K | R | 0.209696722 | 0.172211853 | 273 | L | M | 0.207369167 | 0.150911133 |
| 470 | A | P | 0.209480997 | 0.11945606 | 695 | E | G | 0.207324806 | 0.170023455 |
| 389 | K | R | 0.209459216 | 0.190864781 | 662 | N | S | 0.207198335 | 0.146245893 |
| 233 | M | K | 0.209263613 | 0.148910419 | 102 | P | R | 0.2071 03872 | 0.104479817 |
| 846 | V | A | 0.209194154 | 0.132301095 | 212 | E | G | 0.207077093 | 0.167731322 |
| 803 | Q | R | 0.209112961 | 0.157007924 | 118 | G | V | 0.20699607 | 0.113451465 |
| 594 | -EF | GRI | 0.209067243 | 0.142920346 | 841 | G | R | 0.20698149 | 0.160303912 |
| 418 | D | Y | 0.208952621 | 0.201914561 | 501 | S | R | 0.206963691 | 0.188972116 |
| 424 | I | N | 0.208940616 | 0.184257414 | 402 | L | M | 0.206953352 | 0.103953797 |
| 152 | ----- | TNYFG (SEQ | 0.208921679 | 0.069015043 | 642 | ------- | EVLDSSN | 0.206944663 | 0.088763805 |
| | | ID NO: 3756) | | | | | (SEQ ID NO: | | |
| | | | | | | | 3406) | | |
| 184 | ------- | SLGKFGQ | 0.208921679 | 0.145515626 | 448 | S | C | 0.205480956 | 0.165327281 |
| | | (SEQ ID NO: | | | | | | | |
| | | 3717) | | | | | | | |
| 944 | ---- | QTNK (SEQ | 0.208921679 | 0.115799997 | 341 | V | L | 0.205333121 | 0.121382241 |
| | | ID NO: 3652) | | | | | | | |
| 435 | IK | DR | 0.208921679 | 0.100379476 | 351 | K | [stop] | 0.205260708 | 0.137391414 |
| 926 | LN | PV | 0.208921679 | 0.122257143 | 408 | K | [stop] | 0.205233141 | 0.101895161 |
| 31 | L | P | 0.208720548 | 0.120146815 | 626 | R | [stop] | 0.204917321 | 0.133170214 |
| 426 | ------ | KKVEGLS | 0.206944663 | 0.120828794 | 426 | K | N | 0.204813329 | 0.115277631 |
| | | (SEQ ID NO: | | | | | | | |
| | | 3507) | | | | | | | |
| 273 | -- | LA | 0.206944663 | 0.200099204 | 217 | N | D | 0.204605492 | 0.15571936 |
| 631 | AL | DR | 0.206944663 | 0.132545056 | 55 | P | A | 0.204494052 | 0.203454056 |
| 75 | E | V | 0.206746722 | 0.108008381 | 979 | L--E | VSSK (SEQ | 0.204463305 | 0.104199954 |
| | | | | | | | ID NO: 3797) | | |
| 159 | ------ | NVSEHER | 0.206678079 | 0.108971025 | 789 | EG | GD | 0.204429605 | 0.094907378 |
| | | (SEQ ID NO: | | | | | | | |
| | | 3606) | | | | | | | |
| 974 | - | K | 0.206678079 | 0.087902725 | 174 | P | H | 0.204410022 | 0.192547659 |
| 13 | L | T | 0.206678079 | 0.17404612 | 37 | T | I | 0.20435056 | 0.108024009 |
| 135 | P | L | 0.206613655 | 0.11493052 | 230 | D | Y | 0.204310577 | 0.163888419 |
| 576 | D | N | 0.206571359 | 0.197674836 | 369 | A | D | 0.204246596 | 0.143255593 |
| 396 | -- | YQ | 0.206474109 | 0.165665557 | 567 | V | L | 0.204221782 | 0.133245956 |
| 426 | K | R | 0.206261752 | 0.175070461 | 356 | E | G | 0.204079788 | 0.096784994 |
| 720 | R | S | 0.206187746 | 0.130762963 | 826 | E | G | 0.204045427 | 0.079692638 |
| 731 | D | H | 0.206140141 | 0.18515674 | 234 | ------ | GAVASF | 0.203921342 | 0.148635343 |
| | | | | | | | (SEQ ID NO: | | |
| | | | | | | | 3423) | | |
| 792 | ----- | PSKTY (SEQ | 0.206037621 | 0.119445689 | 791 | - | LP | 0.203921342 | 0.086381396 |
| | | ID NO: 3623) | | | | | | | |
| 470 | ------ | ADKDEFC | 0.206037621 | 0.160849031 | 550 | F | Y | 0.203856294 | 0.154808557 |
| | | (SEQ ID NO: | | | | | | | |
| | | 3306) | | | | | | | |
| 846 | ---- | VEGQ (SEQ | 0.205946011 | 0.115023996 | 139 | Y | H | 0.203748432 | 0.112669732 |
| | | ID NO: 3773) | | | | | | | |
| 730 | ----- | ADDMV | 0.205946011 | 0.203904239 | 842 | K | E | 0.203739019 | 0.14619773 |
| | | (SEQ ID NO: | | | | | | | |
| | | 3303) | | | | | | | |
| 195 | F | S | 0.205931771 | 0.0997168 | 565 | E | D | 0.203689065 | 0.115937226 |
| 763 | R | G | 0.205931024 | 0.177755816 | 667 | IA | TV | 0.203650432 | 0.146532587 |
| 668 | A | G | 0.205831825 | 0.181720031 | 554 | ----- | RFYTV (SEQ | 0.203650432 | 0.085651298 |
| | | | | | | | ID NO: 3666) | | |
| 123 | T | I | 0.205810457 | 0.169798366 | 481 | ----- | KLQKW | 0.203650432 | 0.173739202 |
| | | | | | | | (SEQ ID NO: | | |
| | | | | | | | 3514) | | |
| 394 | A | G | 0.205790009 | 0.129212763 | 64 | A | V | 0.203579261 | 0.147026682 |
| 776 | T | N | 0.205770287 | 0.088016724 | 429 | E | K | 0.203478388 | 0.197959656 |
| 779 | E | D | 0.205703015 | 0.117547264 | 659 | R | W | 0.203469266 | 0.155374384 |
| 787 | A | G | 0.205542455 | 0.113825299 | 644 | L | M | 0.201626647 | 0.191409491 |
| 775 | Y | [stop] | 0.203457477 | 0.112309611 | 326 | K | E | 0.201516415 | 0.172628702 |
| 420 | A | P | 0.203276202 | 0.137871454 | 584 | P | T | 0.201277532 | 0.157595812 |
| 844 | -- | LK | 0.20327417 | 0.108693201 | 216 | G | A | 0.201151425 | 0.135718161 |
| 543 | KK | DR | 0.20327417 | 0.081409516 | 158 | C | R | 0.200895575 | 0.132515505 |
| 483 | QK | DR | 0.203103924 | 0.108226373 | 557 | T | P | 0.20079665 | 0.175823626 |
| 661 | E---N | DHSRD (SEQ | 0.203103924 | 0.080468187 | 615 | ------- | VIEKTLY | 0.20079665 | 0.14533527 |
| | | ID NO: 3355) | | | | | (SEQ ID NO: | | |
| | | | | | | | 3779) | | |
| 591 | -------- | QGREFIWN | 0.203103924 | 0.127711804 | 121 | R | I | 0.200425228 | 0.146944719 |
| | | (SEQ ID NO: | | | | | | | |
| | | 3637) | | | | | | | |
| 434 | ----- | HIKLE (SEQ | 0.203103924 | 0.128782985 | 67 | N | K | 0.200404848 | 0.19495599 |
| | | ID NO: 3461) | | | | | | | |
| 192 | A | D | 0.203101012 | 0.088663269 | 258 | E | G | 0.200396788 | 0.144009482 |
| 979 | LE | VW | 0.203097285 | 0.114357374 | 232 | -- | CM | 0.200312143 | 0.13867079 |
| 905 | V | E | 0.2029568 | 0.158582123 | 526 | -- | LN | 0.200312143 | 0.15960761 |
| 648 | N | K | 0.202865781 | 0.076554962 | 202 | -RE | SSS | 0.200312143 | 0.113603268 |
| 811 | N | D | 0.202736819 | 0.184175153 | 68 | K | T | 0.200238961 | 0.196349346 |
| 573 | F | Y | 0.202703202 | 0.143842683 | 448 | S | Y | 0.200204468 | 0.144800694 |
| 388 | K | E | 0.202623765 | 0.1173393 | 837 | --- | TTI | 0.200162181 | 0.089943784 |
| 265 | K | [stop] | 0.202622408 | 0.159704419 | 158 | ----- | CNVSE (SEQ | 0.200162181 | 0.088327822 |
| | | | | | | | ID NO: 3339) | | |
| 511 | Q | E | 0.202512176 | 0.199826141 | 796 | ------- | YLSKTLA | 0.200048174 | 0.1285851 |
| | | | | | | | (SEQ ID NO: | | |
| | | | | | | | 3852) | | |
| 375 | E | Q | 0.202480508 | 0.162732896 | 276 | -- | PK | 0.200048174 | 0.079289415 |
| 106 | N | K | 0.202431652 | 0.125127347 | 801 | ---- | LAQY (SEQ | 0.200048174 | 0.196038539 |
| | | | | | | | ID NO: 3540) | | |
| 52 | E | G | 0.202421366 | 0.17180627 | 651 | ----- | PMNLI (SEQ | 0.200048174 | 0.135317157 |
| | | | | | | | ID NO: 3620) | | |
| 597 | W | [stop] | 0.202346989 | 0.135138719 | 756 | - | N | 0.200048174 | 0.172777109 |
| 153 | N | K | 0.202320957 | 0.084739162 | 149 | ------ | KPHTNY | 0.200048174 | 0.109852809 |
| | | | | | | | (SEQ ID NO: | | |
| | | | | | | | 3521) | | |
| 471 | D | E | 0.202309983 | 0.069685161 | 494 | -- | FA | 0.200048174 | 0.123840308 |
| 486 | Y | H | 0.202105792 | 0.189019359 | 181 | V | I | 0.19996686 | 0.166465973 |
| 732 | D | V | 0.202045584 | 0.172766987 | 616 | I | M | 0.19990025 | 0.183539616 |
| 833 | T | I | 0.202003023 | 0.114654955 | 264 | -- | LK | 0.198353725 | 0.107390522 |
| 220 | A | D | 0.201986226 | 0.167650811 | 296 | ---- | VVAQ (SEQ | 0.198353725 | 0.116995821 |
| | | | | | | | ID NO: 3835) | | |
| 386 | D | G | 0.201893421 | 0.144223833 | 152 | T | I | 0.198333224 | 0.117839718 |
| 271 | N | K | 0.201821721 | 0.136225013 | 720 | R | G | 0.198275202 | 0.180739318 |
| 236 | VA | -C | 0.201781577 | 0.118494484 | 236 | V | L | 0.198162379 | 0.091047961 |
| 661 | E | Q | 0.201717523 | 0.126595353 | 903 | R | [stop] | 0.197764314 | 0.184873287 |
| 227 | A | - | 0.199865011 | 0.119483676 | 190 | Q | [stop] | 0.197676182 | 0.135507554 |
| 866 | S | R | 0.199834101 | 0.105100812 | 19 | TK | PG | 0.197606812 | 0.087295898 |
| 664 | ------ | PAVIALT | 0.199723054 | 0.116432821 | 554 | R | [stop] | 0.197270424 | 0.119115645 |
| | | (SEQ ID NO: | | | | | | | |
| | | 3612) | | | | | | | |
| 955 | R | W | 0.199719648 | 0.122422647 | 63 | R | K | 0.197266572 | 0.156106069 |
| 507 | G | A | 0.199700659 | 0.133738835 | 671 | D | Y | 0.197186873 | 0.193857965 |
| 925 | ---- | ALNI (SEQ | 0.199681554 | 0.112069534 | 380 | YL | T[stop] | 0.197159823 | 0.186882164 |
| | | ID NO: 3320) | | | | | | | |
| 419 | --- | EAW | 0.199681554 | 0.151874009 | 210 | P | R | 0.197120998 | 0.088119535 |
| 663 | I | N | 0.199667187 | 0.147345549 | 637 | T | S | 0.196993711 | 0.074085124 |
| 845 | K | R | 0.199649448 | 0.119477749 | 657 | I | M | 0.196919314 | 0.094328263 |
| 782 | L | V | 0.199620025 | 0.156520261 | 458 | -- | AK | 0.196819897 | 0.136384351 |
| 173 | K | E | 0.199587002 | 0.098249426 | 304 | V | F | 0.196773726 | 0.171052025 |
| 615 | ------- | VIEKTLYN | 0.199584873 | 0.182641156 | 263 | N | K | 0.196728929 | 0.082784462 |
| | | (SEQ ID NO: | | | | | | | |
| | | 3780) | | | | | | | |
| 630 | P | A | 0.199530215 | 0.103804567 | 601 | L | V | 0.196677335 | 0.163553469 |
| 446 | AQ | DR | 0.199529716 | 0.10633379 | 545 | I | N | 0.196522854 | 0.15815205 |
| 374 | Q | [stop] | 0.199329379 | 0.131990493 | 571 | VN | AV | 0.196419899 | 0.093569564 |
| 778 | M | K | 0.199291554 | 0.158456568 | 284 | ----- | PHTKE (SEQ | 0.196419899 | 0.146831822 |
| | | | | | | | ID NO: 3618) | | |
| 858 | R | S | 0.199265103 | 0.108121324 | 163 | -HE | PTR | 0.196323235 | 0.180126799 |
| 579 | N | I | 0.19915895 | 0.103520322 | 57 | P | L | 0.196165872 | 0.129483671 |
| 63 | R | G | 0.199095742 | 0.127135026 | 659 | R | P | 0.196165872 | 0.140190097 |
| 646 | S | I | 0.199062518 | 0.104634011 | 784 | A | P | 0.196137855 | 0.183129066 |
| 90 | K | E | 0.199052878 | 0.198240775 | 323 | Q | H | 0.196115938 | 0.150227482 |
| 203 | -- | ES | 0.19897765 | 0.14607778 | 763 | R | W | 0.195967691 | 0.113028792 |
| 439 | E | Q | 0.198907882 | 0.179263601 | 257 | N | Y | 0.195936425 | 0.189617104 |
| 621 | Y | C | 0.198885865 | 0.125823263 | 125 | s | G | 0.19588405 | 0.126337645 |
| 310 | Q | H | 0.198723557 | 0.146313995 | 787 | A | T | 0.195855224 | 0.170500255 |
| 60 | N | K | 0.198659421 | 0.192782927 | 213 | Q | L | 0.195810372 | 0.164285983 |
| 299 | Q | R | 0.1986231 | 0.112149973 | 979 | --- | VSS | 0.195756097 | 0.115771783 |
| 279 | T | s | 0.198506775 | 0.126696973 | 440 | E | Q | 0.192625703 | 0.16228978 |
| 278 | I | N | 0.198457202 | 0.188794837 | 698 | K | N | 0.192440231 | 0.067040488 |
| 462 | -- | FV | 0.198353725 | 0.132924725 | 757 | L | Q | 0.192392703 | 0.11735809 |
| 466 | G | D | 0.195631404 | 0.128114426 | 446 | ---- | AQSK (SEQ | 0.192307738 | 0.188279486 |
| | | | | | | | ID NO: 3329) | | |
| 388 | K | R | 0.195529616 | 0.155892093 | 91 | D | Y | 0.192222499 | 0.161107527 |
| 767 | R | K | 0.195477683 | 0.182282632 | 65 | N | K | 0.192152721 | 0.086051749 |
| 673 | E | V | 0.195473785 | 0.111723182 | 228 | L | Q | 0.192019982 | 0.075226208 |
| 864 | D | Y | 0.195306139 | 0.092331083 | 107 | I | N | 0.191587572 | 0.153969194 |
| 885 | T | K | 0.195258477 | 0.131521124 | 307 | N | S | 0.191540821 | 0.186358955 |
| 856 | Y | C | 0.195214677 | 0.129834532 | 944 | QT | PV | 0.191451442 | 0.133263263 |
| 205 | N | S | 0.194826059 | 0.070507432 | 526 | ------ | LNLYLI (SEQ | 0.191451442 | 0.098341333 |
| | | | | | | | ID NO: 3565) | | |
| 696 | S | R | 0.194740876 | 0.106074027 | 750 | -A | LS | 0.191451442 | 0.07841082 |
| 498 | A | V | 0.194435389 | 0.108630638 | 651 | --- | PMN | 0.191451442 | 0.159749911 |
| 281 | P | H | 0.194325757 | 0.164586878 | 370 | ----- | GYKRQ (SEQ | 0.191451442 | 0.172523736 |
| | | | | | | | ID NO: 3456) | | |
| 106 | N | D | 0.194156411 | 0.113601316 | 654 | L | V | 0.191441378 | 0.100236525 |
| 756 | --- | NLS | 0.194120313 | 0.113317678 | 332 | P | L | 0.191427852 | 0.132400599 |
| 591 | ---- | QGRE (SEQ | 0.194120313 | 0.089464524 | 724 | S | G | 0.191322798 | 0.152424888 |
| | | ID NO: 3635) | | | | | | | |
| 572 | N | D | 0.194049735 | 0.182872987 | 206 | H | D | 0.191266107 | 0.183831734 |
| 762 | G | S | 0.193891502 | 0.138436771 | 594 | E | D | 0.191101272 | 0.114552929 |
| 41 | R | [stop] | 0.193882715 | 0.149226534 | 525 | K | E | 0.190973602 | 0.101119046 |
| 370 | G | D | 0.193873435 | 0.131402011 | 576 | D | E | 0.190942249 | 0.134849057 |
| 58 | I | T | 0.193827338 | 0.18015548 | 663 | I | V | 0.190923863 | 0.098130963 |
| 64 | A | S | 0.193814684 | 0.163559402 | 225 | G | A | 0.190920356 | 0.167486936 |
| 203 | E | G | 0.193809853 | 0.182009134 | 227 | A | V | 0.190541259 | 0.158522801 |
| 318 | E | K | 0.193618764 | 0.182298755 | 539 | ---- | KLRF (SEQ | 0.190525892 | 0.118424918 |
| | | | | | | | ID NO: 3515) | | |
| 867 | V | L | 0.193526313 | 0.149480344 | 336 | ------- | RQANEVD | 0.190525892 | 0.095546149 |
| | | | | | | | (SEQ ID NO: | | |
| | | | | | | | 3676) | | |
| 343 | W | [stop] | 0.193259223 | 0.086409476 | 511 | --- | QYN | 0.190525892 | 0.10542285 |
| 920 | ---- | AAEQ (SEQ | 0.1932196 | 0.09807778 | 182 | -- | TY | 0.190525892 | 0.095282059 |
| | | ID NO: 3298) | | | | | | | |
| 559 | I | N | 0.193172208 | 0.185545361 | 955 | R | K | 0.190477708 | 0.163763612 |
| 577 | D | E | 0.193102893 | 0.104761592 | 936 | ------ | RSQEYK | 0.188141846 | 0.120467426 |
| | | | | | | | (SEQ ID NO: | | |
| | | | | | | | 3686) | | |
| 721 | K | N | 0.193081281 | 0.123219324 | 428 | VE | AV | 0.188141846 | 0.111936388 |
| 767 | R | S | 0.19293341 | 0.180949858 | 419 | ---- | EAWE (SEQ | 0.188141846 | 0.161004571 |
| | | | | | | | ID NO: 3378) | | |
| 353 | L | P | 0.192916533 | 0.142447603 | 148 | ------ | GKPFITN | 0.188141846 | 0.126152225 |
| | | | | | | | (SEQ ID NO: | | |
| | | | | | | | 3437) | | |
| 662 | N | D | 0.192798707 | 0.113762689 | 972 | ------ | VWICPA | 0.188141846 | 0.100559027 |
| | | | | | | | (SEQ ID NO: | | |
| | | | | | | | 3838) | | |
| 87 | E | G | 0.192780117 | 0.1542337 | 328 | F | S | 0.188082476 | 0.152191585 |
| 347 | V | G | 0.192656101 | 0.11936042 | 596 | I | N | 0.188043065 | 0.141822306 |
| 669 | L | V | 0.190343627 | 0.076107876 | 482 | L | V | 0.187880246 | 0.186391629 |
| 492 | K | Q | 0.190290589 | 0.150334427 | 582 | I | V | 0.18725447 | 0.136748728 |
| 721 | K | E | 0.190242607 | 0.123347897 | 699 | E | Q | 0.187137878 | 0.176072109 |
| 389 | K | E | 0.190239723 | 0.177951808 | 758 | S | I | 0.18709104 | 0.158068821 |
| 619 | T | I | 0.190153498 | 0.116807589 | 113 | 1 | N | 0.187005943 | 0.142849404 |
| 93 | V | E | 0.190153374 | 0.163133537 | 968 | K | E | 0.186636923 | 0.128956962 |
| 336 | R | G | 0.190122687 | 0.099072113 | 168 | ----- | LLSPH (SEQ | 0.186576707 | 0.08269231 |
| | | | | | | | ID NO: 3560) | | |
| 878 | N | K | 0.190097445 | 0.16631012 | 833 | TGWM (SEQ | PAG[stop] | 0.186576707 | 0.125195246 |
| | | | | | | ID NO: 3289) | | | |
| 847 | -- | EG | 0.190063819 | 0.165413398 | 272 | ------- | GLAFPK | 0.186576707 | 0.060722091 |
| | | | | | | | (SEQ ID NO: | | |
| | | | | | | | 3442) | | |
| 481 | --- | KLQ | 0.190063819 | 0.144467422 | 529 | ----- | YLIIN (SEQ | 0.186576707 | 0.104569212 |
| | | | | | | | ID NO: 3851) | | |
| 655 | I | N | 0.190024208 | 0.138898845 | 261 | ------- | LANLKD | 0.186576707 | 0.081389931 |
| | | | | | | | (SEQ ID NO: | | |
| | | | | | | | 3539) | | |
| 696 | S- | TG | 0.189908515 | 0.068382259 | 884 | W | [stop] | 0.18656617 | 0.16960295 |
| 55 | P | R | 0.189907461 | 0.115309052 | 719 | S | F | 0.186508523 | 0.176978743 |
| 269 | S | N | 0.18989023 | 0.150359662 | 825 | L | M | 0.185209061 | 0.126954087 |
| 210 | P | L | 0.189875815 | 0.142379934 | 727 | K | M | 0.185134776 | 0.155871835 |
| 798 | S | Y | 0.18982788 | 0.189131471 | 28 | M | K | 0.1848853 | 0.176098567 |
| 258 | E | K | 0.189676636 | 0.183203558 | 404 | H | R | 0.184633168 | 0.163423927 |
| 190 | Q | P | 0.189645523 | 0.168321089 | 394 | A | T | 0.184555363 | 0.1424277 |
| 377 | L | V | 0.189542806 | 0.136436344 | 581 | I | F | 0.184470581 | 0.083013305 |
| 500 | N | S | 0.189535073 | 0.180860478 | 766 | K | M | 0.184394313 | 0.16735316 |
| 295 | N | S | 0.18951855 | 0.108197323 | 547 | P | L | 0.184346525 | 0.155161861 |
| 974 | K | [stop] | 0.189482309 | 0.139647592 | 275 | F | S | 0.184250266 | 0.085183481 |
| 54 | I | V | 0.189429698 | 0.1555694 | 537 | G | V | 0.184185986 | 0.146420736 |
| 736 | N | D | 0.189336313 | 0.075796871 | 873 | S | N | 0.184149692 | 0.143102895 |
| 505 | I | N | 0.189099927 | 0.151637022 | 198 | -I | CL | 0.184139991 | 0.106675461 |
| 396 | Y | H | 0.189044775 | 0.129353397 | 639 | --- | ERR | 0.184139991 | 0.11669463 |
| 117 | D | V | 0.188915066 | 0.132090825 | 287 | -K | CL | 0.184067988 | 0.105370778 |
| 8 | K | M | 0.188755388 | 0.159809948 | 404 | H | N | 0.183958455 | 0.132891407 |
| 699 | E | K | 0.188739566 | 0.092771182 | 710 | ----- | VEQRR (SEQ | 0.183918384 | 0.104439918 |
| | | | | | | | ID NO: 3776) | | |
| 132 | C | G | 0.188700628 | 0.133537793 | 889 | S | P | 0.183788189 | 0.164091129 |
| 338 | A | V | 0.188698117 | 0.151434141 | 144 | V | L | 0.183743996 | 0.065170935 |
| 641 | R | [stop] | 0.188367145 | 0.11062471 | 165 | R | K | 0.183736362 | 0.17610787 |
| 208 | V | L | 0.188333358 | 0.080207667 | 28 | M | V | 0.183560659 | 0.134087452 |
| 207 | P | T | 0.188302368 | 0.15553127 | 611 | A | T | 0.183558778 | 0.136945744 |
| 879 | N | K | 0.186386792 | 0.12079248 | 148 | GK | DR | 0.183483799 | 0.153480995 |
| 712 | Q | L | 0.186379419 | 0.129128012 | 515 | A | C | 0.183483799 | 0.109594032 |
| 583 | L | P | 0.186146799 | 0.156442099 | 367 | N | S | 0.183341948 | 0.159877593 |
| 323 | ---- | QRLK (SEQ | 0.186069265 | 0.110701992 | 868 | E | K | 0.183187044 | 0.163165035 |
| | | ID NO: 3648) | | | | | | | |
| 358 | ---- | KEDG (SEQ | 0.18604741 | 0.119601341 | 306 | L | Q | 0.183120006 | 0.156397405 |
| | | ID NO: 3492) | | | | | | | |
| 835 | -- | WM | 0.18604741 | 0.100790291 | 216 | G | D | 0.183066489 | 0.119789101 |
| 839 | ------- | INGKELK | 0.18604741 | 0.115878922 | 728 | N | Y | 0.183065668 | 0.166304554 |
| | | (SEQ ID NO: | | | | | | | |
| | | 3477) | | | | | | | |
| 463 | V | E | 0.186017541 | 0.06776571 | 879 | N | I | 0.183004606 | 0.128653405 |
| 299 | Q | H | 0.185842115 | 0.085070655 | 126 | G | V | 0.182789208 | 0.179342988 |
| 832 | A | C | 0.185822701 | 0.103905008 | 35 | V | M | 0.182763396 | 0.156289233 |
| 127 | F | Y | 0.185786991 | 0.140080792 | 443 | S | N | 0.182633222 | 0.162446869 |
| 159 | N | S | 0.185693031 | 0.145375399 | 951 | N | D | 0.182629417 | 0.175906154 |
| 532 | -- | IN | 0.185685948 | 0.088889817 | 410 | G | S | 0.182624091 | 0.128840332 |
| 439 | ----- | EERRS (SEQ | 0.185685948 | 0.095520154 | 382 | SS | CL | 0.180218478 | 0.105067529 |
| | | ID NO: 3382) | | | | | | | |
| 152 | -- | TN | 0.185685948 | 0.085877547 | 369 | AG | DS | 0.180218478 | 0.132171137 |
| 684 | --- | LGN | 0.18563709 | 0.122810431 | 757 | LS | PV | 0.180218478 | 0.120148198 |
| 718 | Y | [stop] | 0.185557954 | 0.073476523 | 674 | -------- | GCPLSRFK | 0.180218478 | 0.119094301 |
| | | | | | | | (SEQ ID NO: | | |
| | | | | | | | 3425) | | |
| 585 | L | P | 0.185474446 | 0.130833458 | 418 | -- | DE | 0.180218478 | 0.162709755 |
| 85 | W | R | 0.185353654 | 0.134359698 | 702 | ------- | RTIQAAK | 0.180179308 | 0.102882749 |
| | | | | | | | (SEQ ID NO: | | |
| | | | | | | | 3693) | | |
| 931 | ----- | SWLFL (SEQ | 0.185304071 | 0.113870586 | 81 | L | P | 0.180116381 | 0.137095425 |
| | | ID NO: 3735) | | | | | | | |
| 543 | ---- | KKIK (SEQ | 0.185304071 | 0.066752877 | 939 | --- | EYK | 0.18007812 | 0.13192478 |
| | | ID NO: 3501) | | | | | | | |
| 547 | ------- | PEAFEAN | 0.185304071 | 0.089391329 | 31 | L | Q | 0.180015666 | 0.152602881 |
| | | (SEQ ID NO: | | | | | | | |
| | | 3615) | | | | | | | |
| 91 | D | G | 0.1853036 | 0.092089443 | 213 | ----- | QIGGN (SEQ | 0.179890016 | 0.080439406 |
| | | | | | | | ID NO: 3638) | | |
| 766 | K | R | 0.185284272 | 0.110005204 | 379 | -- | PY | 0.179789203 | 0.118280148 |
| 461 | ----- | SFVIE (SEQ | 0.185264915 | 0.156592075 | 331 | F | Y | 0.179617168 | 0.14637274 |
| | | ID NO: 3698) | | | | | | | |
| 950 | ----- | GNTDK (SEQ | 0.185264915 | 0.154386625 | 540 | L | M | 0.179584486 | 0.167412262 |
| | | ID NO: 3446) | | | | | | | |
| 233 | M | V | 0.182567289 | 0.115088116 | 693 | I | V | 0.179569128 | 0.124539552 |
| 96 | M | L | 0.182378018 | 0.128312349 | 776 | T | S | 0.179453432 | 0.075575874 |
| 753 | ------ | IFANLS (SEQ | 0.182269944 | 0.088037483 | 264 | L | V | 0.179340275 | 0.144429387 |
| | | ID NO: 3472) | | | | | | | |
| 634 | V | A | 0.182243984 | 0.121794563 | 547 | P | R | 0.179333799 | 0.110886672 |
| 556 | Y | S | 0.182208476 | 0.102238152 | 820 | D | E | 0.179273983 | 0.124243775 |
| 972 | ------- | VWKPAV | 0.182135365 | 0.122971859 | 604 | E | K | 0.17907609 | 0.153006263 |
| | | (SEQ ID NO: | | | | | | | |
| | | 3839)[stop] | | | | | | | |
| 716 | G | D | 0.182118038 | 0.088377906 | 651 | P | S | 0.17907294 | 0.16496086 |
| 419 | E | G | 0.182093842 | 0.165354368 | 382 | S | C | 0.179061797 | 0.042397129 |
| 145 | N | K | 0.181832601 | 0.074663212 | 680 | F | Y | 0.179026865 | 0.083849485 |
| 652 | M | R | 0.181725898 | 0.15882275 | 552 | A | V | 0.178983921 | 0.137645246 |
| 183 | Y | [stop] | 0.181723054 | 0.087766244 | 693 | I | F | 0.178916903 | 0.17080226 |
| 229 | S | R | 0.18162155 | 0.118611624 | 151 | HT | LS | 0.178787645 | 0.11267363 |
| 589 | K | E | 0.181594685 | 0.120760487 | 190 | ----- | QRALD (SEQ | 0.178787645 | 0.150480322 |
| | | | | | | | ID NO: 3645) | | |
| 304 | V | I | 0.181591972 | 0.14363826 | 208 | ----- | VKPLE (SEQ | 0.178787645 | 0.112763983 |
| | | | | | | | ID NO: 3783) | | |
| 873 | S | C | 0.181321853 | 0.144241543 | 194 | D | V | 0.178645393 | 0.146182868 |
| 114 | P | S | 0.181260379 | 0.131437002 | 767 | RT | Sc | 0.176164273 | 0.119651092 |
| 100 | A | S | 0.181149523 | 0.170663024 | 678 | S | N | 0.176147348 | 0.146692604 |
| 413 | W | [stop] | 0.181066052 | 0.139390154 | 817 | T | A | 0.176123605 | 0.120992816 |
| 166 | L | M | 0.180963828 | 0.128703075 | 635 | A | G | 0.176061926 | 0.119367224 |
| 496 | ------ | IEAENS (SEQ | 0.180890191 | 0.096196015 | 212 | E | A | 0.175873239 | 0.11085302 |
| | | ID NO: 3468) | | | | | | | |
| 504 | D | V | 0.180843532 | 0.116307526 | 821 | Y | [stop] | 0.175384143 | 0.118184345 |
| 199 | H | Q | 0.180819165 | 0.098967075 | 447 | Q | R | 0.175284629 | 0.123528707 |
| 675 | C | W | 0.180770613 | 0.172891211 | 257 | N | S | 0.175186561 | 0.099304683 |
| 94 | G | S | 0.180639091 | 0.140246364 | 618 | K | R | 0.175178956 | 0.153225543 |
| 212 | E | D | 0.180617877 | 0.126552831 | 217 | N | S | 0.175170771 | 0.153898212 |
| 557 | T | N | 0.180519556 | 0.15369828 | 852 | Y | [stop] | 0.175104531 | 0.090584521 |
| 753 | I | S | 0.180492647 | 0.165598334 | 255 | K | R | 0.175069831 | 0.070668507 |
| 872 | L | V | 0.180432435 | 0.164444609 | 430 | --- | GLS | 0.175035484 | 0.093564105 |
| 596 | ------ | IWNDLL | 0.180218478 | 0.160627748 | 827 | ---- | KLKK (SEQ | 0.175035484 | 0.069987475 |
| | | (SEQ ID NO: | | | | | ID NO: 3510) | | |
| | | 3487) | | | | | | | |
| 163 | H | R | 0.178633884 | 0.108142143 | 796 | --- | YLS | 0.175035484 | 0.092544675 |
| 383 | S | I | 0.178486259 | 0.158810182 | 414 | --------- | GKVYDEAW | 0.175035484 | 0.140128399 |
| | | | | | | | E (SEQ ID | | |
| | | | | | | | NO: 3441) | | |
| 156 | G | D | 0.178426488 | 0.134868493 | 547 | ----- | PEAFE (SEQ | 0.175035484 | 0.118947618 |
| | | | | | | | ID NO: 3614) | | |
| 234 | G | E | 0.178414368 | 0.12320748 | 186 | ------ | GKFGQR | 0.175035484 | 0.092907507 |
| | | | | | | | (SEQ ID NO: | | |
| | | | | | | | 3435) | | |
| 804 | Y | [stop] | 0.178116642 | 0.169884859 | 580 | L | R | 0.174993228 | 0.092760152 |
| 582 | I | N | 0.177915368 | 0.151449157 | 422 | E | K | 0.174900558 | 0.171745203 |
| 655 | I | T | 0.177824888 | 0.131979099 | 285 | H | Y | 0.174862549 | 0.137793142 |
| 129 | C | Y | 0.177764169 | 0.131217004 | 737 | T | I | 0.174757975 | 0.115488534 |
| 20 | K | [stop] | 0.177744686 | 0.162022223 | 455 | W | G | 0.174674459 | 0.156270727 |
| 852 | Y | C | 0.177655192 | 0.126363222 | 401 | L | P | 0.174440338 | 0.064966394 |
| 179 | E | Q | 0.177438027 | 0.163530401 | 953 | - | DKR | 0.174181069 | 0.090682808 |
| 365 | W | S | 0.177330558 | 0.12784352 | 953 | ---- | DKRA (SEQ | 0.174181069 | 0.085814279 |
| | | | | | | | ID NO: 3359) | | |
| 245 | D | E | 0.177288135 | 0.128142583 | 360 | D | N | 0.174161173 | 0.117286104 |
| 593 | R | G | 0.177150053 | 0.165372274 | 520 | K | E | 0.174117735 | 0.143263172 |
| 838 | T | S | 0.177144418 | 0.166381063 | 255 | K | M | 0.171890748 | 0.139268571 |
| 979 | LE[stop]G | VSSR (SEQ | 0.177037198 | 0.160568847 | 675 | -- | CP | 0.171877476 | 0.064917248 |
| | | ID NO: 3834) | | | | | | | |
| 265 | K | E | 0.176890073 | 0.124809095 | 853 | Y | C | 0.171733581 | 0.087723362 |
| 440 | E | D | 0.176868582 | 0.097257257 | 631 | A | V | 0.171731995 | 0.15053602 |
| 107 | I | M | 0.176863119 | 0.14397234 | 668 | A | V | 0.171647872 | 0.129168631 |
| 22 | A | P | 0.176753805 | 0.123959084 | 508 | F | S | 0.17126701 | 0.136692573 |
| 292 | A | G | 0.176665583 | 0.159949136 | 925 | AL | DR | 0.17104041 | 0.083554381 |
| 803 | Q | [stop] | 0.176624558 | 0.101059884 | 437 | -- | LE | 0.17104041 | 0.06885585 |
| 329 | P | S | 0.176586746 | 0.173503743 | 853 | -- | YN | 0.17104041 | 0.123300185 |
| 196 | Y | [stop] | 0.176517802 | 0.122355941 | 797 | ------ | LSKTLA | 0.17104041 | 0.064415402 |
| | | | | | | | (SEQ ID NO: | | |
| | | | | | | | 3574) | | |
| 758 | S | N | 0.176368261 | 0.089480066 | 815 | --- | TIT | 0.17104041 | 0.104377719 |
| 298 | A | T | 0.176357721 | 0.087659893 | 462 | --FV | ERL[stop] | 0.17104041 | 0.089353273 |
| 333 | L | V | 0.176333899 | 0.163860363 | 471 | -- | DK | 0.17104041 | 0.0730883 |
| 518 | W | R | 0.176185261 | 0.104632883 | 418 | ----- | DEAWE (SEQ | 0.170904662 | 0.126366449 |
| | | | | | | | ID NO: 3348) | | |
| 459 | KA | -V | 0.176164273 | 0.103778218 | 213 | --- | QIG | 0.170882441 | 0.117196646 |
| 192 | AL | DR | 0.176164273 | 0.079837153 | 703 | ---- | TIQA (SEQ | 0.170763645 | 0.147647998 |
| | | | | | | | ID NO: 3750) | | |
| 979 | LE----[stop]G | VSSKDLQA | 0.176164273 | 0.074531926 | 356 | E | A | 0.170659559 | 0.127216719 |
| | | (SEQ ID NO: | | | | | | | |
| | | 3810) | | | | | | | |
| 35 | VMT | ETA | 0.176164273 | 0.104758915 | 869 | L | V | 0.170596065 | 0.1158133 |
| 145 | N | D | 0.174107257 | 0.119744646 | 106 | NI | TV | 0.170299453 | 0.164756763 |
| 819 | ---- | ADYD (SEQ | 0.174068679 | 0.17309276 | 160 | V | L | 0.170273865 | 0.111449611 |
| | | ID NO: 3307) | | | | | | | |
| 561 | K | [stop] | 0.174057181 | 0.086009056 | 163 | H | Q | 0.170101095 | 0.104599592 |
| 761 | F | S | 0.17403349 | 0.168753775 | 210 | P | T | 0.170021527 | 0.150133417 |
| 563 | S | P | 0.173902999 | 0.138700996 | 748 | QD | R- | 0.169874659 | 0.074658631 |
| 70 | L | P | 0.173882613 | 0.120818159 | 775 | ------ | YTRMED | 0.169874659 | 0.080414628 |
| | | | | | | | (SEQ ID NO: | | |
| | | | | | | | 3859) | | |
| 24 | K | [stop] | 0.173808747 | 0.113872328 | 513 | N | I | 0.169811112 | 0.150139289 |
| 834 | G | A | 0.173722333 | 0.117168406 | 743 | -- | YY | 0.169783049 | 0.088429509 |
| 167 | I | N | 0.173700086 | 0.14772793 | 467 | ------- | LKEADKD | 0.169783049 | 0.163043441 |
| | | | | | | | (SEQ ID NO: | | |
| | | | | | | | 3556) | | |
| 496 | -------- | IEAENSILD | 0.173653508 | 0.110162475 | 859 | | QNVVK (SEQ | 0.167565632 | 0.122604368 |
| | | (SEQ ID NO: | | | | | ID NO: 3643) | | |
| | | 3470) | | | | | | | |
| 618 | K | [stop] | 0.173508668 | 0.101750483 | 719 | S | P | 0.167206156 | 0.083551442 |
| 297 | V | E | 0.173261294 | 0.132967549 | 712 | Q | R | 0.167205037 | 0.147128575 |
| 426 | K | E | 0.173245682 | 0.081642461 | 964 | F | S | 0.166884399 | 0.138397154 |
| 182 | T | K | 0.173138422 | 0.156579716 | 359 | E | G | 0.16680448 | 0.139659272 |
| 660 | G | S | 0.17299716 | 0.158169348 | 191 | R | K | 0.166577954 | 0.144007057 |
| 805 | T | S | 0.172972548 | 0.12868971 | 339 | N | D | 0.166374831 | 0.157063101 |
| 458 | A | S | 0.172827968 | 0.144714634 | 212 | E | K | 0.166305352 | 0.157035199 |
| 731 | D | V | 0.172739834 | 0.130565896 | 413 | WG | LS | 0.166270685 | 0.125303472 |
| 829 | K | E | 0.172710008 | 0.121812751 | 149 | -- | KP | 0.166270685 | 0.076773688 |
| 859 | Q | [stop] | 0.172627299 | 0.130823394 | 284 | ---- | PHTK (SEQ | 0.166270685 | 0.139854804 |
| | | | | | | | ID NO: 3617) | | |
| 305 | -- | NL | 0.172611068 | 0.12831984 | 146 | D | N | 0.166006779 | 0.113823305 |
| 178 | - | DE | 0.172611068 | 0.108355628 | 686 | N | D | 0.165853975 | 0.141480032 |
| 652 | M | V | 0.172566944 | 0.106266804 | 492 | K | R | 0.16571672 | 0.088451245 |
| 582 | I | M | 0.172413921 | 0.144870464 | 580 | LI | PV | 0.165563978 | 0.079217211 |
| 335 | E | G | 0.172324707 | 0.120749484 | 661 | --- | ENI | 0.165563978 | 0.126675099 |
| 940 | -- | YK | 0.172247171 | 0.104630004 | 829 | K | R | 0.165378823 | 0.103172827 |
| 450 | A | D | 0.172235862 | 0.15659478 | 608 | L | V | 0.165024412 | 0.161094218 |
| 187 | K | T | 0.172165735 | 0.159986695 | 451 | --- | ALT | 0.164823895 | 0.158152194 |
| 289 | GI | AV | 0.172163889 | 0.117287191 | 581 | II | TV | 0.164823895 | 0.074002626 |
| 579 | NL | DR | 0.172163889 | 0.094383078 | 297 | ---- | VAQI (SEQ | 0.164823895 | 0.107420642 |
| | | | | | | | ID NO: 3765) | | |
| 843 | E | G | 0.172115298 | 0.163114025 | 783 | - | T | 0.164823895 | 0.135845679 |
| 259 | K | E | 0.171933606 | 0.128545463 | 496 | I | V | 0.164665656 | 0.140996169 |
| 663 | -I | CL | 0.169783049 | 0.106475808 | 979 | LE[stop]G | VSSE (SEQ | 0.164491714 | 0.145714149 |
| | | | | | | | ID NO: 3795) | | |
| 803 | ------ | QYTSKT | 0.169772888 | 0.094792337 | 932 | ---- | WLFL (SEQ | 0.164491714 | 0.083188044 |
| | | (SEQ ID NO: | | | | | ID NO: 3841) | | |
| | | 3655) | | | | | | | |
| 808 | ------ | TCSNCG | 0.169772888 | 0.089412307 | 637 | ------ | TFERRE | 0.164491714 | 0.152633112 |
| | | (SEQ ID NO: | | | | | (SEQ ID NO: | | |
| | | 3739) | | | | | 3745) | | |
| 845 | K | E | 0.169715078 | 0.127028772 | 325 | --- | LKG | 0.164491714 | 0.125129505 |
| 552 | A | T | 0.169382091 | 0.146396839 | 764 | ------ | QGKRTFM | 0.163440941 | 0.098647738 |
| | | | | | | | (SEQ ID NO: | | |
| | | | | | | | 3634) | | |
| 476 | C | F | 0.169278987 | 0.093974927 | 107 | I | T | 0.163178218 | 0.154967966 |
| 711 | E | D | 0.169174495 | 0.118203075 | 633 | FVAL (SEQ | LWP[stop] | 0.163026367 | 0.076347451 |
| | | | | | | ID NO: 3259) | | | |
| 631 | A | S | 0.169116909 | 0.130583861 | 213 | -- | QI | 0.163026367 | 0.09979216 |
| 303 | W | [stop] | 0.169003266 | 0.078930757 | 186 | ----- | GKFGQ (SEQ | 0.163026367 | 0.114909103 |
| | | | | | | | ID NO: 3434) | | |
| 561 | K | I | 0.168954178 | 0.166308652 | 592 | G | D | 0.162807696 | 0.109433096 |
| 157 | -- | RC | 0.168739459 | 0.094824256 | 257 | N | K | 0.162725471 | 0.091658038 |
| 721 | K | R | 0.168620063 | 0.147491806 | 473 | DE | YH | 0.162404215 | 0.086992333 |
| 614 | R | [stop] | 0.168568195 | 0.15863634 | 975 | P | A | 0.162340126 | 0.074611129 |
| 611 | A | D | 0.168315642 | 0.157590847 | 833 | T | A | 0.162275301 | 0.096163195 |
| 78 | K | [stop] | 0.168282214 | 0.125424128 | 871 | R | S | 0.162178581 | 0.080758991 |
| 917 | ---- | ETHA (SEQ | 0.168207257 | 0.122439321 | 909 | ----- | FVCLN (SEQ | 0.162125073 | 0.14885021 |
| | | ID NO: 3398) | | | | | ID NO: 3421) | | |
| 756 | NL | DR | 0.168207257 | 0.079944251 | 341 | -- | VD | 0.162125073 | 0.111287809 |
| 678 | S | G | 0.168124453 | 0.111226188 | 57 | PI | DS | 0.162125073 | 0.110736083 |
| 525 | K | I | 0.16804127 | 0.142310409 | 83 | VY | AV | 0.162125073 | 0.121259318 |
| 653 | N | K | 0.167953422 | 0.124668308 | 643 | --- | VLD | 0.162125073 | 0.148280778 |
| 37 | T | N | 0.16794635 | 0.137106698 | 561 | K | N | 0.161973573 | 0.145314105 |
| 174 | P | S | 0.167775884 | 0.122107474 | 349 | N | K | 0.161796683 | 0.105713204 |
| 756 | ---- | NLSR (SEQ | 0.167679572 | 0.073550026 | 318 | E | R | 0.161659235 | 0.066441966 |
| | | ID NO: 3594) | | | | | | | |
| 168 | ------ | LLSPHK | 0.167679572 | 0.081935755 | 554 | -- | RF | 0.161611946 | 0.149093192 |
| | | (SEQ ID NO: | | | | | | | |
| | | 3561) | | | | | | | |
| 160 | ------- | VSEHERLI | 0.167679572 | 0.116191677 | 505 | I | F | 0.161489243 | 0.076235653 |
| | | (SEQ ID NO: | | | | | | | |
| | | 3791) | | | | | | | |
| 630 | ---- | PALF (SEQ | 0.164491714 | 0.073996533 | 102 | P | T | 0.161386248 | 0.119400583 |
| | | ID NO: 3610) | | | | | | | |
| 343 | ----- | WWDMV | 0.164491714 | 0.076194534 | 514 | CA | LS | 0.16113532 | 0.083183292 |
| | | (SEQ ID NO: | | | | | | | |
| | | 3846) | | | | | | | |
| 642 | -- | EV | 0.164491714 | 0.162646605 | 979 | ------ | VSSKDLQ | 0.161025471 | 0.108550491 |
| | | | | | | | (SEQ ID NO: | | |
| | | | | | | | 3809) | | |
| 419 | ----- | EAWER (SEQ | 0.164491714 | 0.082157078 | 445 | D | Y | 0.161008394 | 0.118993907 |
| | | ID NO: 3379) | | | | | | | |
| 360 | -- | DG | 0.164491714 | 0.073133393 | 143 | Q | K | 0.160693826 | 0.130109004 |
| 408 | K | E | 0.16446662 | 0.067392631 | 547 | P | S | 0.160635883 | 0.144061844 |
| 48 | R | G | 0.164301321 | 0.157884797 | 29 | K | N | 0.158279304 | 0.142748603 |
| 613 | G | D | 0.164218988 | 0.127296459 | 372 | K | R | 0.158267712 | 0.11920003 |
| 175 | ----- | EANDE (SEQ | 0.164149182 | 0.111610409 | 275 | F | L | 0.158241303 | 0.120299703 |
| | | ID NO: 3377) | | | | | | | |
| 671 | D | E | 0.164120916 | 0.112217289 | 741 | L | P | 0.158158865 | 0.120228264 |
| 794 | ------- | KTYLSKT | 0.16411942 | 0.087804343 | 430 | G | V | 0.158115277 | 0.126566194 |
| | | (SEQ ID NO: | | | | | | | |
| | | 3531) | | | | | | | |
| 599 | ------ | DLLSLE | 0.16411942 | 0.120903184 | 921 | --- | AEQ | 0.158108573 | 0.11103467 |
| | | (SEQ ID NO: | | | | | | | |
| | | 3364) | | | | | | | |
| 58 | I- | LS | 0.16411942 | 0.094001227 | 242 | K | E | 0.158032112 | 0.1512035 |
| 826 | E | D | 0.163807302 | 0.112540279 | 148 | GK | RQ | 0.158026029 | 0.155853601 |
| 889 | S | [stop] | 0.163771981 | 0.149267099 | 295 | -- | NV | 0.157603522 | 0.100157866 |
| 199 | ---H | PRLY (SEQ | 0.163715064 | 0.07899198 | 876 | ---- | SVNN (SEQ | 0.157603522 | 0.131358152 |
| | | ID NO: 3622) | | | | | ID NO: 3732) | | |
| 916 | FET | VQA | 0.163715064 | 0.085074401 | 215 | G | A | 0.157466168 | 0.125711629 |
| 496 | ------- | IEAENSI | 0.163715064 | 0.073631578 | 319 | A | V | 0.15742503 | 0.144655841 |
| | | (SEQ ID NO: | | | | | | | |
| | | 3469) | | | | | | | |
| 164 | ---- | ERLI (SEQ ID | 0.163715064 | 0.124419929 | 222 | G | A | 0.157400391 | 0.107390901 |
| | | NO: 3394) | | | | | | | |
| 345 | D | G | 0.16357556 | 0.12500461 | 523 | V | D | 0.157098281 | 0.069302906 |
| 134 | Q | [stop] | 0.163522049 | 0.142382805 | 753 | ------- | IFANLSR | 0.157085986 | 0.062378414 |
| | | | | | | | (SEQ ID NO: | | |
| | | | | | | | 3473) | | |
| 43 | R | Q | 0.160624353 | 0.132247177 | 177 | N | S | 0.157058654 | 0.117427271 |
| 317 | D | E | 0.160609141 | 0.14140596 | 461 | S | R | 0.157014829 | 0.122688776 |
| 807 | K | [stop] | 0.160484146 | 0.104229856 | 823 | R | T | 0.156977695 | 0.125466793 |
| 572 | N | S | 0.160431799 | 0.062377966 | 427 | K | M | 0.156963925 | 0.118535881 |
| 644 | LD | PV | 0.160242602 | 0.128569608 | 111 | K | [stop] | 0.156885345 | 0.101390983 |
| 699 | EK | DR | 0.160242602 | 0.092172248 | 253 | V | L | 0.156787797 | 0.082680225 |
| 850 | I | V | 0.160226988 | 0.152692033 | 91 | D | V | 0.156758895 | 0.14763673 |
| 100 | AQ | LS | 0.160110772 | 0.101933413 | 71 | T | I | 0.156624998 | 0.127600056 |
| 558 | VI | CL | 0.160110772 | 0.10892714 | 592 | ------ | GREFIW | 0.156575371 | 0.050528735 |
| | | | | | | | (SEQ ID NO: | | |
| | | | | | | | 3450) | | |
| 270 | -- | AN | 0.160110772 | 0.124579798 | 847 | ----- | EGQIT (SEQ | 0.156575371 | 0.108055014 |
| | | | | | | | ID NO: 3386) | | |
| 979 | LE[stop]GS- | VSSKDLQAS | 0.160110772 | 0.049257177 | 111 | KL | S[stop] | 0.156575371 | 0.112953961 |
| | PGIK (SEQ ID | NT (SEQ ID | | | | | | | |
| | NO: | NO: 3816) | | | | | | | |
| | 3279)[stop] | | | | | | | | |
| 484 | K---WYGD | NSSLSASF | 0.160110772 | 0.077521171 | 979 | L-E[stop] | VSSN (SEQ | 0.156575371 | 0.054922359 |
| | (SEQ ID NO: | (SEQ ID NO: | | | | | ID NO: 3829) | | |
| | 3274) | 3602) | | | | | | | |
| 205 | NH | LS | 0.160110772 | 0.08695461 | 717 | G | E | 0.15414714 | 0.124750031 |
| 281 | P | C | 0.160110772 | 0.141761431 | 667 | I | V | 0.154117319 | 0.147646705 |
| 939 | E | R | 0.160110772 | 0.106121188 | 623 | ----- | RRTRQ (SEQ | 0.153993707 | 0.122323206 |
| | | | | | | | ID NO: 3682) | | |
| 672 | - | S | 0.160110772 | 0.105653932 | 773 | R | G | 0.153915262 | 0.146586561 |
| 894 | ------- | SLLKKRFS | 0.160110772 | 0.071577892 | 433 | -- | KH | 0.153881949 | 0.097541884 |
| | | (SEQ ID NO: | | | | | | | |
| | | 3722) | | | | | | | |
| 199 | HV | T[stop] | 0.160110772 | 0.129212095 | 35 | V | G | 0.153666817 | 0.124448628 |
| 47 | L | Q | 0.159718064 | 0.101565653 | 211 | L | V | 0.153538313 | 0.134546484 |
| 262 | A | V | 0.159650297 | 0.156994685 | 26 | G | D | 0.15349539 | 0.149545585 |
| 788 | ------ | YEGLPS | 0.159522485 | 0.129386966 | 279 | ----- | TLPPQ (SEQ | 0.15339361 | 0.125011235 |
| | | (SEQ ID NO: | | | | | ID NO: 3754) | | |
| | | 3848) | | | | | | | |
| 529 | Y | N | 0.159442162 | 0.135286632 | 664 | ------ | PAVIAL | 0.15339361 | 0.13972264 |
| | | | | | | | (SEQ ID NO: | | |
| | | | | | | | 3611) | | |
| 604 | E | V | 0.159292857 | 0.097301034 | 377 | ---- | LLPY (SEQ | 0.15339361 | 0.12480719 |
| | | | | | | | ID NO: 3559) | | |
| 284 | P | S | 0.159001205 | 0.153355474 | 53 | N | D | 0.15332875 | 0.117758231 |
| 750 | A | D | 0.158401706 | 0.125762435 | 140 | K | N | 0.153228737 | 0.097346381 |
| 950 | G | A | 0.158324371 | 0.153957854 | 694 | GE | DR | 0.153190779 | 0.097274205 |
| 688 | T | I | 0.158292674 | 0.119969439 | 741 | ---- | LLYY (SEQ | 0.153190779 | 0.13376095 |
| | | | | | | | ID NO: 3562) | | |
| 203 | ------ | ESNHPV | 0.156575371 | 0.141927058 | 592 | ----- | GREFI (SEQ | 0.153190779 | 0.103123693 |
| | | (SEQ ID NO: | | | | | ID NO: 3449) | | |
| | | 3396) | | | | | | | |
| 230 | DA | LS | 0.156575371 | 0.105363533 | 684 | ------ | LGNPTHI | 0.153147895 | 0.112048537 |
| | | | | | | | (SEQ ID NO: | | |
| | | | | | | | 3550) | | |
| 408 | ----- | KHGED (SEQ | 0.156575371 | 0.140706352 | 532 | --- | INY | 0.153147895 | 0.072663729 |
| | | ID NO: 3497) | | | | | | | |
| 606 | ------- | GSLKLAN | 0.156575371 | 0.154364417 | 311 | K | N | 0.153086255 | 0.08609524 |
| | | (SEQ ID NO: | | | | | | | |
| | | 3454) | | | | | | | |
| 166 | L | Q | 0.156435151 | 0.079474192 | 678 | ----- | SRFKD (SEQ | 0.152422378 | 0.09122337 |
| | | | | | | | ID NO: 3728) | | |
| 213 | Q | H | 0.156012357 | 0.091435578 | 969 | LK | PV | 0.152422378 | 0.0541377 |
| 447 | Q | E | 0.155900092 | 0.095629939 | 419 | EAWERIDKK | RPGRESTRR | 0.152422378 | 0.081179935 |
| | | | | | | V (SEQ ID | W (SEQ ID | | |
| | | | | | | NO: 3256) | NO: 3674) | | |
| 689 | H | P | 0.155877877 | 0.131928361 | 670 | -- | TD | 0.152422378 | 0.096788119 |
| 335 | E | Q | 0.155876225 | 0.110366115 | 383 | --- | SEE | 0.152422378 | 0.066189551 |
| 84 | Y | D | 0.155784728 | 0.135489779 | 880 | --- | DIS | 0.15109455 | 0.085164607 |
| 531 | I | N | 0.155410746 | 0.152604803 | 296 | VV | DR | 0.15109455 | 0.140218943 |
| 103 | A | S | 0.155352263 | 0.149390311 | 293 | YN | DS | 0.15109455 | 0.094395956 |
| 661 | E | V | 0.155230224 | 0.090301063 | 359 | ED | AV | 0.15109455 | 0.062026733 |
| 865 | ------- | LSVELDR | 0.15478543 | 0.145114034 | 210 | PL | RQ | 0.15109455 | 0.109823159 |
| | | (SEQ ID NO: | | | | | | | |
| | | 3579) | | | | | | | |
| 677 | LS | PV | 0.15478543 | 0.108120931 | 758 | S- | TG | 0.15109455 | 0.105413113 |
| 570 | E | G | 0.154599098 | 0.10691093 | 232 | CM | LS | 0.15109455 | 0.096388212 |
| 762 | G | D | 0.154432235 | 0.117428168 | 930 | RSWLFL | EAGCS (SEQ | 0.15109455 | 0.077157167 |
| | | | | | | (SEQ ID NO: | ID NO: | | |
| | | | | | | 3287) | 3376)[stop] | | |
| 177 | N | K | 0.15431964 | 0.1416948 | 886 | KG | C- | 0.15109455 | 0.085064934 |
| 484 | K | N | 0.154291635 | 0.117621744 | 594 | EF | DC | 0.15109455 | 0.055097165 |
| 592 | GRE-- | DNQVG (SEQ | 0.154254957 | 0.077027283 | 140 | K | [stop] | 0.150604639 | 0.124522684 |
| | | ID NO: 3368) | | | | | | | |
| 704 | ----- | IQAAK (SEQ | 0.154254957 | 0.108682368 | 979 | LE[stop]GS- | VSSKDI (SEQ | 0.150527572 | 0.113935287 |
| | | ID NO: 3480) | | | | | ID NO: 3803) | | |
| 285 | ----- | HTKEG (SEQ | 0.154254957 | 0.106587271 | 979 | L-E[stop]G | VSSKA (SEQ | 0.150527572 | 0.106493096 |
| | | ID NO: 3464) | | | | | ID NO: 3798) | | |
| 721 | KY | TV | 0.154254957 | 0.124126134 | 851 | T | A | 0.150513073 | 0.138774627 |
| 650 | ------- | KPMNLIG | 0.154254957 | 0.151047576 | 615 | V | A | 0.150425208 | 0.101961366 |
| | | (SEQ ID NO: | | | | | | | |
| | | 3524) | | | | | | | |
| 403 | ---- | LHLE (SEQ | 0.152422378 | 0.132942463 | 359 | - | E | 0.150399286 | 0.136024193 |
| | | ID NO: 3551) | | | | | | | |
| 389 | KG | TV | 0.152422378 | 0.11037889 | 508 | ------ | FSKQYN | 0.150399286 | 0.049469473 |
| | | | | | | | (SEQ ID NO: | | |
| | | | | | | | 3416) | | |
| 850 | ----- | ITYYN (SEQ | 0.152422378 | 0.102611165 | 202 | R-------- | SSSLASGL | 0.150399286 | 0.07744146 |
| | | ID NO: 3484) | | | | | (SEQ ID NO: | | |
| | | | | | | | 3731)[stop] | | |
| 230 | ------- | DACMGAV | 0.152422378 | 0.082337669 | 884 | ----- | WTKGR | 0.150399286 | 0.084711675 |
| | | (SEQ ID NO: | | | | | (SEQ ID NO: | | |
| | | 3343) | | | | | 3844) | | |
| 461 | ---- | SFVI (SEQ ID | 0.152422378 | 0.085894307 | 399 | ------ | GDLLLH | 0.150399286 | 0.08514719 |
| | | NO: 3697) | | | | | (SEQ ID NO: | | |
| | | | | | | | 3426) | | |
| 673 | E- | DR | 0.152422378 | 0.059554386 | 39 | D | G | 0.150354378 | 0.13986784 |
| 257 | N | D | 0.152411625 | 0.106853984 | 891 | E | V | 0.150263535 | 0.113865674 |
| 590 | R | G | 0.152081011 | 0.117905973 | 450 | A | P | 0.150166455 | 0.146935336 |
| 737 | T | N | 0.151886476 | 0.142783247 | 240 | ---- | LTKY (SEQ | 0.147451251 | 0.080958956 |
| | | | | | | | ID NO: 3581) | | |
| 790 | G | E | 0.151825437 | 0.098317165 | 942 | KY | NC | 0.147451251 | 0.116243971 |
| 831 | T | S | 0.151806143 | 0.14386859 | 47 | LR | C- | 0.147451251 | 0.058888218 |
| 906 | QE | PV | 0.151695593 | 0.100183043 | 807 | KT | -C | 0.147451251 | 0.120603495 |
| 99 | V | D | 0.151565952 | 0.12300149 | 603 | LE | PV | 0.147451251 | 0.066385351 |
| 959 | --- | ETW | 0.151393972 | 0.086210639 | 873 | --- | SEE | 0.147451251 | 0.078348652 |
| 520 | K | R | 0.151365824 | 0.113621271 | 15 | KD | R- | 0.147451251 | 0.123855007 |
| 852 | Y | N | 0.151328449 | 0.137543743 | 206 | HP | DS | 0.147451251 | 0.064383902 |
| 444 | E | G | 0.151257656 | 0.118296919 | 599 | DL | -- | 0.147451251 | 0.079608104 |
| 147 | --- | KGK | 0.15109455 | 0.054833005 | 979 | L-E[stop]GS | VSSKDP | 0.147451251 | 0.049212446 |
| | | | | | | | (SEQ ID NO: | | |
| | | | | | | | 3822) | | |
| 171 | -- | PH | 0.15109455 | 0.08380172 | 979 | LE[stop]GS- | VSSNDLQAS | 0.147451251 | 0.067765787 |
| | | | | | | PGIK (SEQ ID | NK (SEQ ID | | |
| | | | | | | NO: | NO: 3833) | | |
| | | | | | | 3279)[stop] | | | |
| 925 | --- | ALN | 0.15109455 | 0.138412128 | 448 | -- | SK | 0.147451251 | 0.090898875 |
| 539 | ----- | KLRFK (SEQ | 0.15109455 | 0.128926028 | 505 | I- | LS | 0.147451251 | 0.077683234 |
| | | ID NO: 3516) | | | | | | | |
| 334 | ------- | VERQANE | 0.15109455 | 0.059721295 | 398 | FG | SV | 0.147451251 | 0.073631355 |
| | | (SEQ ID NO: | | | | | | | |
| | | 3777) | | | | | | | |
| 484 | KW | TG | 0.15109455 | 0.091510022 | 512 | -Y | DS | 0.147451251 | 0.05128316 |
| 848 | G- | AV | 0.15109455 | 0.104352239 | 345 | ---- | DMVC (SEQ | 0.147451251 | 0.06441585 |
| | | | | | | | ID NO: 3366) | | |
| 236 | ------ | VASFLT | 0.15109455 | 0.088006138 | 177 | ND-- | FTG[stop] | 0.147451251 | 0.085413531 |
| | | (SEQ ID NO: | | | | | | | |
| | | 3767) | | | | | | | |
| 429 | E | D | 0.149933575 | 0.107236607 | 36 | MT | C- | 0.147451251 | 0.118494367 |
| 77 | K | E | 0.148931072 | 0.079170957 | 953 | D- | AV | 0.147451251 | 0.040719542 |
| 259 | ------- | KRLANLKD | 0.148805792 | 0.108390156 | 451 | AL | DR | 0.147451251 | 0.096339405 |
| | | (SEQ ID NO: | | | | | | | |
| | | 3528) | | | | | | | |
| 978 | [stop]L | GI | 0.148805792 | 0.119775179 | 631 | A | C | 0.147319263 | 0.109020371 |
| 386 | D- | AV | 0.148805792 | 0.079572543 | 848 | G | A | 0.147279724 | 0.093306967 |
| 748 | QD | PV | 0.148805792 | 0.094563395 | 239 | F | S | 0.147177048 | 0.142500129 |
| 609 | KL | DR | 0.148805792 | 0.060702366 | 270 | A | T | 0.147117218 | 0.13621963 |
| 699 | EK | DC | 0.148805792 | 0.122863259 | 352 | K | N | 0.147067273 | 0.12109567 |
| 279 | --- | TLP | 0.148805792 | 0.138832536 | 563 | S | T | 0.147049099 | 0.111696976 |
| 24 | K | M | 0.148782741 | 0.14630409 | 612 | N | K | 0.146927237 | 0.108594483 |
| 798 | S | T | 0.148583442 | 0.105674096 | 569 | M | V | 0.146754771 | 0.119310335 |
| 349 | N | S | 0.148310626 | 0.138528822 | 855 | R | G | 0.144425593 | 0.123370913 |
| 403 | -- | LH | 0.148273333 | 0.102736 | 617 | E | V | 0.144206082 | 0.126166622 |
| 967 | ------ | KKLKEVW | 0.148059201 | 0.11964291 | 918 | -------- | THAAEQAA | 0.143857661 | 0.070236443 |
| | | (SEQ ID NO: | | | | | (SEQ ID NO: | | |
| | | 3504) | | | | | 3749) | | |
| 157 | RC | LS | 0.14801524 | 0.133243315 | 733 | ---- | MVRN (SEQ | 0.143791778 | 0.090612696 |
| | | | | | | | ID NO: 3585) | | |
| 493 | PF | TV | 0.14801524 | 0.059147928 | 217 | NS | TG | 0.143791778 | 0.113745581 |
| 188 | ------ | FGQRALD | 0.14801524 | 0.10137508 | 657 | ----- | IARGE (SEQ | 0.143791778 | 0.039293361 |
| | | (SEQ ID NO: | | | | | ID NO: 3466) | | |
| | | 3412) | | | | | | | |
| 898 | KR | TG | 0.14801524 | 0.120213578 | 533 | N | S | 0.14375365 | 0.085993529 |
| 186 | -- | GK | 0.14801524 | 0.114746024 | 185 | ------- | LGKFGQRA | 0.14367777 | 0.094952199 |
| | | | | | | | (SEQ ID NO: | | |
| | | | | | | | 3548) | | |
| 328 | F- | LS | 0.14801524 | 0.071716609 | 616 | ------- | IEKTLYN | 0.14367777 | 0.110151228 |
| | | | | | | | (SEQ ID NO: | | |
| | | | | | | | 3471) | | |
| 204 | ------ | SNHPVKP | 0.14801524 | 0.094645672 | 668 | ------ | ALTDPE | 0.14367777 | 0.113895553 |
| | | (SEQ ID NO: | | | | | (SEQ ID NO: | | |
| | | 3724) | | | | | 3323) | | |
| 314 | -- | IG | 0.14801524 | 0.075655093 | 259 | ---- | KRLA (SEQ | 0.14367777 | 0.070148108 |
| | | | | | | | ID NO: 3527) | | |
| 422 | ER | AV | 0.14801524 | 0.044733928 | 175 | E- | DR | 0.14367777 | 0.049065425 |
| 64 | AN | DS | 0.14801524 | 0.108571015 | 610 | ------ | LANGRV | 0.14367777 | 0.105216814 |
| | | | | | | | (SEQ ID NO: | | |
| | | | | | | | 3537) | | |
| 855 | -- | RY | 0.14801524 | 0.108772293 | 507 | ------- | GFSKQYN | 0.14367777 | 0.101689858 |
| | | | | | | | (SEQ ID NO: | | |
| | | | | | | | 3430) | | |
| 504 | D | E | 0.147876758 | 0.098656217 | 487 | --- | GDL | 0.14367777 | 0.046711447 |
| 342 | D | H | 0.147844774 | 0.140125334 | 731 | DD | CL | 0.14367777 | 0.067816779 |
| 86 | EE | DR | 0.147451251 | 0.143531987 | 265 | KD | R- | 0.14367777 | 0.130304386 |
| 940 | -Y | SV | 0.14673352 | 0.076906931 | 386 | --- | DRK | 0.14367777 | 0.092432212 |
| 794 | KT | NC | 0.14673352 | 0.093083088 | 790 | ----- | GLPSK (SEQ | 0.14367777 | 0.104428158 |
| | | | | | | | ID NO: 3444) | | |
| 487 | ---- | GDLR (SEQ | 0.14673352 | 0.141269601 | 147 | -------- | KGKPHTNY | 0.140217655 | 0.060731949 |
| | | ID NO: 3427) | | | | | (SEQ ID NO: | | |
| | | | | | | | 3496) | | |
| 717 | -- | GY | 0.14673352 | 0.129086357 | 979 | LE[stop]GS- | VSSKDV | 0.140217655 | 0.126849347 |
| | | | | | | | (SEQ ID NO: | | |
| | | | | | | | 3824) | | |
| 468 | ---- | KEAD (SEQ | 0.14673352 | 0.112176586 | 342 | - | D | 0.140217655 | 0.083180031 |
| | | ID NO: 3490) | | | | | | | |
| 102 | P | L | 0.146729077 | 0.094784801 | 701 | ------ | QRTIQA | 0.140217655 | 0.094973524 |
| | | | | | | | (SEQ ID NO: | | |
| | | | | | | | 3650) | | |
| 462 | F | V | 0.146714745 | 0.123539268 | 588 | G | R | 0.140077599 | 0.123307802 |
| 291 | E | Q | 0.146533408 | 0.078647294 | 248 | L | V | 0.139838145 | 0.132091481 |
| 657 | ------ | IDRGEN | 0.146511494 | 0.145489762 | 641 | R | G | 0.139811399 | 0.120984089 |
| | | (SEQ ID NO: | | | | | | | |
| | | 3467) | | | | | | | |
| 32 | L | F | 0.146467882 | 0.099225719 | 375 | E | G | 0.13977585 | 0.117490416 |
| 619 | T | N | 0.146372017 | 0.145146105 | 179 | E | K | 0.139614148 | 0.122113279 |
| 355 | N | K | 0.146341962 | 0.141209887 | 285 | --- | HTK | 0.139514563 | 0.076217964 |
| 132 | C | S | 0.146274101 | 0.131138669 | 166 | -- | LI | 0.139514563 | 0.075733937 |
| 831 | T | A | 0.146217161 | 0.113775751 | 786 | ---- | LAYE (SEQ | 0.139514563 | 0.068877295 |
| | | | | | | | ID NO: 3541) | | |
| 868 | E | V | 0.145780526 | 0.143894902 | 274 | AF | TV | 0.139413376 | 0.092095094 |
| 231 | A | P | 0.14576396 | 0.105172115 | 578 | -- | PN | 0.139413376 | 0.112737023 |
| 944 | ----- | QTNKT (SEQ | 0.14564914 | 0.125394667 | 775 | ----- | YTRME (SEQ | 0.13869596 | 0.096841774 |
| | | ID NO: 3653) | | | | | ID NO: 3858) | | |
| 236 | ----- | VASFL (SEQ | 0.14564914 | 0.09085897 | 838 | TING (SEQ | PSTA (SEQ | 0.13869596 | 0.135948561 |
| | | ID NO: 3766) | | | | ID NO: 3290) | ID NO: 3624) | | |
| 709 | -- | EV | 0.14564914 | 0.119119066 | 75 | E | K | 0.138622423 | 0.112055782 |
| 865 | L | P | 0.145527367 | 0.10928669 | 556 | Y | C | 0.138477684 | 0.131330328 |
| 510 | ---- | KQYN (SEQ | 0.145296444 | 0.112653295 | 98 | R | [stop] | 0.138179687 | 0.102036322 |
| | | ID NO: 3525) | | | | | | | |
| 959 | -- | ET | 0.145296444 | 0.114339851 | 460 | A | T | 0.137813435 | 0.108501414 |
| 414 | G | V | 0.1451247 | 0.140131131 | 111 | K | N | 0.137723187 | 0.11828435 |
| 465 | E | G | 0.144909944 | 0.124547249 | 566 | I | F | 0.137434779 | 0.130961132 |
| 300 | I | T | 0.144877384 | 0.129206612 | 438 | ------ | EEERRS | 0.137192189 | 0.064149715 |
| | | | | | | | (SEQ ID NO: | | |
| | | | | | | | 3380) | | |
| 215 | G | S | 0.144824715 | 0.07809376 | 58 | I | M | 0.13705694 | 0.089110339 |
| 288 | E | G | 0.144744415 | 0.110082872 | 913 | NCGFET | EAAVQA | 0.134611486 | 0.113195929 |
| | | | | | | (SEQ ID NO: | (SEQ ID NO: | | |
| | | | | | | 3282) | 3372) | | |
| 16 | D | N | 0.144678092 | 0.139073977 | 11 | -R | AS | 0.134611486 | 0.123271552 |
| 774 | QY | PV | 0.14367777 | 0.076535556 | 978 | [stop]LE[stop] | YVSSKDLQA | 0.134611486 | 0.087096491 |
| | | | | | | GS-PG (SEQ | (SEQ ID NO: | | |
| | | | | | | ID NO: 3251) | 3864) | | |
| 910 | -- | VC | 0.14367777 | 0.024273265 | 247 | ------ | ILEHQK | 0.134611486 | 0.104206673 |
| | | | | | | | (SEQ ID NO: | | |
| | | | | | | | 3476) | | |
| 484 | KW | DR | 0.14367777 | 0.094175463 | 517 | I | T | 0.134524102 | 0.104605605 |
| 20 | -- | CL | 0.14367777 | 0.08704024 | 18 | N | Y | 0.134422379 | 0.132333464 |
| 847 | -------- | EGQITYYN | 0.14367777 | 0.054370233 | 804 | ---- | YTSK (SEQ | 0.134383084 | 0.102298299 |
| | | (SEQ ID NO: | | | | | ID NO: 3860) | | |
| | | 3389) | | | | | | | |
| 114 | P | L | 0.143623976 | 0.107371623 | 872 | ------- | LSEESVN | 0.134383084 | 0.104954479 |
| | | | | | | | (SEQ ID NO: | | |
| | | | | | | | 3573) | | |
| 294 | N | S | 0.143486731 | 0.084830242 | 743 | Y | H | 0.134286698 | 0.08203884 |
| 473 | D | G | 0.143465301 | 0.122194432 | 250 | H | Q | 0.134238241 | 0.111012466 |
| 376 | A | T | 0.1434567 | 0.101440197 | 268 | A | P | 0.134027791 | 0.098451313 |
| 637 | T | A | 0.143296115 | 0.114711319 | 978 | [stop]LE[stop] | YVSSKDLQ | 0.134010909 | 0.133274253 |
| | | | | | | GSPG (SEQ | (SEQ ID NO: | | |
| | | | | | | ID NO: 3251) | 3863) | | |
| 365 | W | C | 0.143131818 | 0.093254266 | 664 | -- | PA | 0.134010909 | 0.124393367 |
| 559 | I | S | 0.142993499 | 0.107801059 | 979 | LE[stop]G- | VSSND (SEQ | 0.133919467 | 0.126494561 |
| | | | | | | | ID NO: 3830) | | |
| 671 | D | S | 0.142731931 | 0.123439168 | 241 | T | N | 0.133870518 | 0.110803484 |
| 487 | ----- | GDLRGK | 0.14265438 | 0.086040474 | 153 | N | S | 0.133623126 | 0.12555263 |
| | | (SEQ ID NO: | | | | | | | |
| | | 3428) | | | | | | | |
| 211 | LEQIG (SEQ | RNRSA (SEQ | 0.14265438 | 0.100691421 | 196 | Y | H | 0.133619017 | 0.107174466 |
| | ID NO: 3280) | ID NO: 3670) | | | | | | | |
| 26 | GP | CL | 0.14265438 | 0.067388407 | 744 | Y- | LS | 0.133358224 | 0.114892564 |
| 421 | -- | WE | 0.14265438 | 0.084239003 | 633 | F | S | 0.133277029 | 0.122435158 |
| 211 | ---- | LEQI (SEQ ID | 0.14265438 | 0.118588014 | 619 | T | S | 0.133139525 | 0.08963831 |
| | | NO: 3543) | | | | | | | |
| 767 | R | [stop] | 0.141592128 | 0.123403074 | 742 | L | P | 0.133131448 | 0.09127341 |
| 290 | I | N | 0.141531787 | 0.136370873 | 809 | C | [stop] | 0.133028515 | 0.072072201 |
| 774 | Q | [stop] | 0.141517184 | 0.125118121 | 86 | E | D | 0.132733699 | 0.128073996 |
| 341 | V | E | 0.14127686 | 0.094518287 | 473 | D | V | 0.132562245 | 0.055193421 |
| 176 | A | S | 0.140653486 | 0.112098857 | 568 | -- | PM | 0.130626359 | 0.119168349 |
| 562 | K | N | 0.140512419 | 0.126501373 | 362 | K | R | 0.130604026 | 0.105840846 |
| 317 | D | H | 0.140493859 | 0.124148887 | 359 | E | V | 0.130475561 | 0.064946527 |
| 941 | ------ | KKYQTN | 0.140217655 | 0.077001548 | 426 | ---- | KKVE (SEQ | 0.130424348 | 0.109290243 |
| | | (SEQ ID NO: | | | | | ID NO: 3506) | | |
| | | 3508) | | | | | | | |
| 826 | E | K | 0.136937076 | 0.066669616 | 300 | IV | DR | 0.130424348 | 0.08495594 |
| 955 | R | T | 0.136388186 | 0.086919652 | 893 | -- | LS | 0.130424348 | 0.106896252 |
| 400 | ----- | DLLLH (SEQ | 0.136321349 | 0.064628042 | 256 | KN | TV | 0.130424348 | 0.057621352 |
| | | ID NO: 3361) | | | | | | | |
| 163 | -------- | HERLILL | 0.136321349 | 0.117792482 | 767 | ---- | RTFM (SEQ | 0.130424348 | 0.06446722 |
| | | (SEQ ID NO: | | | | | ID NO: 3691) | | |
| | | 3460) | | | | | | | |
| 950 | - | G | 0.136321349 | 0.089773613 | 324 | R | G | 0.13036573 | 0.130162815 |
| 353 | ------- | LINEKKE | 0.136321349 | 0.11384298 | 460 | A | P | 0.129809906 | 0.111386576 |
| | | (SEQ ID NO: | | | | | | | |
| | | 3554) | | | | | | | |
| 469 | -------- | EADKDEFC | 0.136321349 | 0.136235916 | 744 | Y | S | 0.129801283 | 0.120155085 |
| | | (SEQ ID NO: | | | | | | | |
| | | 3373) | | | | | | | |
| 298 | ------ | AQIVIW | 0.136321349 | 0.124259801 | 297 | V | L | 0.1296923 | 0.098130283 |
| | | (SEQ ID NO: | | | | | | | |
| | | 3328) | | | | | | | |
| 967 | --- | KKL | 0.136321349 | 0.087024226 | 979 | LE | VP | 0.129554025 | 0.068280994 |
| 834 | G | D | 0.136317736 | 0.131556677 | 595 | ------- | FIWNDLL | 0.129554025 | 0.083916268 |
| | | | | | | | (SEQ ID NO: | | |
| | | | | | | | 3414) | | |
| 675 | C | S | 0.135933989 | 0.124817499 | 909 | F | C | 0.129452838 | 0.12013501 |
| 295 | N | D | 0.135903192 | 0.116385268 | 39 | D | N | 0.128914064 | 0.121593627 |
| 489 | L | P | 0.135710175 | 0.113005835 | 263 | N | D | 0.128846416 | 0.111193487 |
| 316 | R | W | 0.135665116 | 0.08159144 | 403 | ------- | LHLEKKH | 0.128586666 | 0.071668629 |
| | | | | | | | (SEQ ID NO: | | |
| | | | | | | | 3553) | | |
| 782 | L | P | 0.135444097 | 0.094158481 | 979 | LE[stop]GS-G | VSSKDLV | 0.128586666 | 0.121567211 |
| | | | | | | | (SEQ ID NO: | | |
| | | | | | | | 3821) | | |
| 252 | K | I | 0.135215444 | 0.118419704 | 876 | ------ | SVNNDI | 0.128586666 | 0.054233667 |
| | | | | | | | (SEQ ID NO: | | |
| | | | | | | | 3733) | | |
| 703 | -- | TI | 0.135116856 | 0.093813019 | 228 | ------ | LSDACMG | 0.128586666 | 0.126842965 |
| | | | | | | | (SEQ ID NO: | | |
| | | | | | | | 3571) | | |
| 671 | --- | DPE | 0.135116856 | 0.117221994 | 701 | ---- | QRTI (SEQ ID | 0.128586666 | 0.098093616 |
| | | | | | | | NO: 3649) | | |
| 763 | R | Q | 0.135073853 | 0.130952104 | 549 | ------- | AFEANRFY | 0.127406426 | 0.084837264 |
| | | | | | | | (SEQ ID NO: | | |
| | | | | | | | 3310) | | |
| 815 | T | S | 0.135026549 | 0.096980291 | 979 | LE[stop]GSPG | VSSKDLQE | 0.127187739 | 0.092227907 |
| | | | | | | I (SEQ ID NO: | (SEQ ID NO: | | |
| | | | | | | 3278) | 3817) | | |
| 141 | L | M | 0.134960075 | 0.098794232 | 445 | D | E | 0.127007554 | 0.122060316 |
| 789 | E | K | 0.134893603 | 0.120008321 | 82 | H | N | 0.126805938 | 0.104486705 |
| 36 | M | L | 0.13488937 | 0.122340012 | 676 | P | L | 0.126754121 | 0.080812602 |
| 278 | I | F | 0.134789571 | 0.111040576 | 951 | ---- | NTDK (SEQ | 0.126641231 | 0.099218396 |
| | | | | | | | ID NO: 3604) | | |
| 358 | K | I | 0.132508402 | 0.120198091 | 979 | LE[stop]GS- | VSSKDLQAS | 0.126641231 | 0.095848514 |
| | | | | | | PGIK (SEQ ID | NN (SEQ ID | | |
| | | | | | | NO: | NO: 3815) | | |
| | | | | | | 3279)[stop] | | | |
| 476 | - | C | 0.132326289 | 0.087739647 | 204 | ---- | SNHP (SEQ | 0.126641231 | 0.07625836 |
| | | | | | | | ID NO: 3723) | | |
| 953 | DK | E- | 0.132326289 | 0.066036843 | 426 | KK | DR | 0.126641231 | 0.097925475 |
| 770 | ------ | MAERQY | 0.132326289 | 0.083381966 | 923 | QAA | PV- | 0.126641231 | 0.093158654 |
| | | (SEQ ID NO: | | | | | | | |
| | | 3584) | | | | | | | |
| 887 | ------- | GRSGEAL | 0.132326289 | 0.072961347 | 101 | QP | ET | 0.126641231 | 0.062121806 |
| | | (SEQ ID NO: | | | | | | | |
| | | 3453) | | | | | | | |
| 630 | P | S | 0.132221835 | 0.08064538 | 942 | K-Y | NCL | 0.126641231 | 0.088910569 |
| 290 | I | T | 0.132066117 | 0.101441805 | 826 | EK | AV | 0.126641231 | 0.091897908 |
| 81 | L | Q | 0.132063026 | 0.114766305 | 292 | ----- | AYNNV (SEQ | 0.126641231 | 0.106376872 |
| | | | | | | | ID NO: 3338) | | |
| 809 | C | F | 0.131888449 | 0.093326725 | 879 | ------ | NDISSWT | 0.126641231 | 0.078787272 |
| | | | | | | | (SEQ ID NO: | | |
| | | | | | | | 3590) | | |
| 497 | ------ | EAENSIL | 0.131863052 | 0.100142921 | 181 | VTYSLGKFG | - | 0.126641231 | 0.089695218 |
| | | (SEQ ID NO: | | | | Q (SEQ ID | SHTAWASSD | | |
| | | 3374) | | | | NO: 3296) | (SEQ ID NO: | | |
| | | | | | | | 3709) | | |
| 717 | ----- | GYSRK (SEQ | 0.131863052 | 0.112950153 | 137 | YV | DR | 0.126641231 | 0.109693213 |
| | | ID NO: 3458) | | | | | | | |
| 386 | ---- | DRKK (SEQ | 0.131863052 | 0.08146183 | 548 | ---- | EAFE (SEQ | 0.126641231 | 0.095888318 |
| | | ID NO: 3369) | | | | | ID NO: 3375) | | |
| 68 | KL | TV | 0.131863052 | 0.070945883 | 670 | ------ | TDPEGCP | 0.12652671 | 0.087582312 |
| | | | | | | | (SEQ ID NO: | | |
| | | | | | | | 3743) | | |
| 700 | KQ | DR | 0.131863052 | 0.063471315 | 344 | -- | WD | 0.12652671 | 0.059784458 |
| 831 | TAT | PPP | 0.131863052 | 0.067816715 | 589 | K | [stop] | 0.126002643 | 0.117169902 |
| 157 | ----- | RCNVS (SEQ | 0.131863052 | 0.080937513 | 670 | T | I | 0.125333365 | 0.115123087 |
| | | ID NO: 3659) | | | | | | | |
| 953 | ------ | DKRAFV | 0.131771442 | 0.07848717 | 843 | E | K | 0.125307936 | 0.1170313 |
| | | (SEQ ID NO: | | | | | | | |
| | | 3360) | | | | | | | |
| 978 | [stop]L | GF | 0.131771442 | 0.061548024 | 209 | --- | KPL | 0.125145098 | 0.058688797 |
| 979 | LE[stop]G | VSCK (SEQ | 0.131568591 | 0.101292375 | 256 | ----- | KNEKR (SEQ | 0.125145098 | 0.118773295 |
| | | ID NO: 3788) | | | | | ID NO: 3517) | | |
| 855 | R | S | 0.131540317 | 0.054730727 | 627 | ------- | QDEPALF | 0.125145098 | 0.11944079 |
| | | | | | | | (SEQ ID NO: | | |
| | | | | | | | 3633) | | |
| 128 | A | T | 0.13150991 | 0.131075942 | 637 | TF | S- | 0.125145098 | 0.075022945 |
| 225 | G | R | 0.131348437 | 0.12857841 | 846 | ------ | VEGQIT | 0.125145098 | 0.095200634 |
| | | | | | | | (SEQ ID NO: | | |
| | | | | | | | 3774) | | |
| 874 | E | D | 0.131154993 | 0.12741404 | 112 | LI | PV | 0.125145098 | 0.061303825 |
| 54 | I | T | 0.130796445 | 0.072189843 | 592 | GRE- | DNQV (SEQ | 0.125145098 | 0.061215515 |
| | | | | | | | ID NO: 3367) | | |
| 797 | -------- | LSKTLAQYT | 0.128586666 | 0.060991971 | 273 | ------- | LAFPKIT | 0.125145098 | 0.062360109 |
| | | (SEQ ID NO: | | | | | (SEQ ID NO: | | |
| | | 3575) | | | | | 3535) | | |
| 14 | VK | AG | 0.128586666 | 0.085310723 | 773 | ---- | RQYT (SEQ | 0.125145098 | 0.098790624 |
| | | | | | | | ID NO: 3680) | | |
| 423 | RI | LS | 0.128586666 | 0.084850033 | 274 | AF | DS | 0.125145098 | 0.089301627 |
| 583 | -- | LP | 0.128586666 | 0.051620503 | 686 | N- | TV | 0.125145098 | 0.106327975 |
| 979 | LE[stop]GS- | VSSNDLQAS | 0.128586666 | 0.102476858 | 549 | - | A | 0.125145098 | 0.111251903 |
| | PGIK (SEQ ID | N (SEQ ID | | | | | | | |
| | NO: 3279) | NO: 3832) | | | | | | | |
| 979 | LE[stop]GS- | FSSKDLQAS | 0.128586666 | 0.093654912 | 615 | --- | VIE | 0.125145098 | 0.115519537 |
| | PGIK (SEQ ID | NK (SEQ ID | | | | | | | |
| | NO: | NO: 3420) | | | | | | | |
| | 3279)[stop] | | | | | | | | |
| 533 | -- | NY | 0.128586666 | 0.127517343 | 486 | Y | [stop] | 0.12498861 | 0.117668911 |
| 563 | ---- | SGEI (SEQ ID | 0.128586666 | 0.112169649 | 479 | E | G | 0.124803485 | 0.119823525 |
| | | NO: 3702) | | | | | | | |
| 979 | L-E[stop]GS | VSSKDH | 0.128586666 | 0.096285329 | 225 | G | E | 0.124549307 | 0.110077498 |
| | | (SEQ ID NO: | | | | | | | |
| | | 3802) | | | | | | | |
| 755 | ---- | ANLS (SEQ | 0.12851771 | 0.091942401 | 123 | T | N | 0.123826195 | 0.091669684 |
| | | ID NO: 3326) | | | | | | | |
| 461 | S | N | 0.128271168 | 0.11452282 | 436 | K | E | 0.123328926 | 0.10928445 |
| 864 | D | E | 0.128210448 | 0.108842691 | 139 | Y | [stop] | 0.123256307 | 0.11429924 |
| 84 | Y | C | 0.128022871 | 0.110536014 | 669 | - | L | 0.119637812 | 0.05675251 |
| 720 | ---- | RKYA (SEQ | 0.127406426 | 0.102905352 | 845 | ------ | KVEGQI | 0.119637812 | 0.06612892 |
| | | ID NO: 3669) | | | | | (SEQ ID NO: | | |
| | | | | | | | 3532) | | |
| 416 | VYDEAWE | CTMRPG | 0.127406426 | 0.059900059 | 400 | ------ | DLLLHL | 0.119637812 | 0.07276695 |
| | (SEQ ID NO: | (SEQ ID NO: | | | | | (SEQ ID NO: | | |
| | 3297) | 3340)- | | | | | 3362) | | |
| 808 | ---- | TCSN (SEQ | 0.127406426 | 0.082184056 | 757 | L | R | 0.119502434 | 0.108713549 |
| | | ID NO: 3738) | | | | | | | |
| 791 | ------ | LPSKTY | 0.127406426 | 0.108127962 | 578 | P | L | 0.119430629 | 0.116829607 |
| | | (SEQ ID NO: | | | | | | | |
| | | 3568) | | | | | | | |
| 162 | ------ | EHERLI (SEQ | 0.127406426 | 0.099109571 | 634 | VA | LS | 0.119372647 | 0.100712827 |
| | | ID NO: 3390) | | | | | | | |
| 858 | ------ | RQNVVKDL | 0.126641231 | 0.065591267 | 510 | K-- | SHL | 0.119372647 | 0.080479619 |
| | | (SEQ ID NO: | | | | | | | |
| | | 3679) | | | | | | | |
| 231 | A | C | 0.126641231 | 0.070173983 | 979 | LE[stop]G | ASSK (SEQ | 0.119372647 | 0.074447954 |
| | | | | | | | ID NO: 3332) | | |
| 898 | KRF | NCL | 0.126641231 | 0.049641927 | 798 | -S | TA | 0.119372647 | 0.036802807 |
| 789 | EG | AV | 0.126641231 | 0.10544887 | 653 | NL | DR | 0.119372647 | 0.061028998 |
| 640 | RR | TG | 0.126641231 | 0.104632778 | 854 | -N | LS | 0.119372647 | 0.074161693 |
| 303 | ----- | WVNLN | 0.126641231 | 0.064376538 | 420 | A | S | 0.119261972 | 0.115184751 |
| | | (SEQ ID NO: | | | | | | | |
| | | 3845) | | | | | | | |
| 640 | R- | TV | 0.126641231 | 0.051697037 | 519 | --- | QKD | 0.119051026 | 0.108753459 |
| 890 | GE | DR | 0.126641231 | 0.058497447 | 600 | LLS | PV- | 0.119011185 | 0.056536344 |
| 513 | ------- | NCAFIWQK | 0.126641231 | 0.110534935 | 271 | ------- | NGLAFPK | 0.119011185 | 0.073725244 |
| | | (SEQ ID NO: | | | | | (SEQ ID NO: | | |
| | | 3589) | | | | | 3592) | | |
| 36 | MT | TV | 0.126641231 | 0.096682191 | 51 | P | L | 0.118978183 | 0.099712186 |
| 979 | -- | AV | 0.126641231 | 0.031136061 | 403 | ----- | LHLEK (SEQ | 0.118963684 | 0.11518549 |
| | | | | | | | ID NO: 3552) | | |
| 607 | --- | SLK | 0.126641231 | 0.117782054 | 457 | ----- | RAKAS (SEQ | 0.118963684 | 0.088377062 |
| | | | | | | | ID NO: 3656) | | |
| 979 | LE[stop]G | FSSK (SEQ | 0.126627253 | 0.064240928 | 776 | ---- | TRME (SEQ | 0.118963684 | 0.083809802 |
| | | ID NO: 3418) | | | | | ID NO: 3759) | | |
| 29 | KT | LS | 0.126627253 | 0.070400509 | 320 | KPLQRL | SHCRD (SEQ | 0.118677331 | 0.073630679 |
| | | | | | | (SEQ ID NO: | ID NO: | | |
| | | | | | | 3270) | 3704)[stop] | | |
| 510 | KQ-Y | SHLQ (SEQ | 0.126602218 | 0.092982894 | 685 | GNPT (SEQ | ATLH (SEQ | 0.118677331 | 0.086334956 |
| | | ID NO: 3705) | | | | ID NO: 3263) | ID NO: 3334) | | |
| 960 | --- | TWQ | 0.12652671 | 0.053263565 | 178 | ---- | DELV (SEQ | 0.118677331 | 0.101525884 |
| | | | | | | | ID NO: 3352) | | |
| 665 | --- | AVI | 0.12652671 | 0.057438099 | 160 | ----- | VSEHE (SEQ | 0.113504256 | 0.099167463 |
| | | | | | | | ID NO: 3789) | | |
| 675 | - | C | 0.12652671 | 0.103567494 | 745 | ----- | AVTQD (SEQ | 0.113504256 | 0.111375922 |
| | | | | | | | ID NO: 3336) | | |
| 451 | ------- | ALTDWLR | 0.12652671 | 0.081452296 | 570 | E | K | 0.1130503 | 0.100973674 |
| | | (SEQ ID NO: | | | | | | | |
| | | 3324) | | | | | | | |
| 805 | ----- | TSKTC (SEQ | 0.12652671 | 0.07786947 | 368 | L | P | 0.111983406 | 0.095724154 |
| | | ID NO: 3760) | | | | | | | |
| 890 | GE | VAKPLLQQ | 0.12652671 | 0.093632788 | 275 | F | Y | 0.111191948 | 0.100665217 |
| | | (SEQ ID NO: | | | | | | | |
| | | 3764) | | | | | | | |
| 885 | -- | TK | 0.12652671 | 0.12280066 | 521 | D | E | 0.111133748 | 0.10058089 |
| 831 | T | N | 0.123113024 | 0.105004336 | 562 | K | E | 0.110566391 | 0.097349138 |
| 147 | ------ | KGKPHTN | 0.123112897 | 0.091739528 | 136 | L | Q | 0.110244812 | 0.107286129 |
| | | (SEQ ID NO: | | | | | | | |
| | | 3495) | | | | | | | |
| 256 | --- | KNE | 0.122844147 | 0.106923843 | 411 | E | G | 0.110174632 | 0.097582202 |
| 179 | EL | A- | 0.122844147 | 0.091584443 | 381 | LS | PV | 0.110164473 | 0.095898615 |
| 406 | ----- | EKKHG (SEQ | 0.122844147 | 0.089153499 | 616 | I | V | 0.109853606 | 0.094001833 |
| | | ID NO: 3392) | | | | | | | |
| 295 | ------ | NVVAQ (SEQ | 0.122844147 | 0.103819809 | 843 | E | R | 0.109803145 | 0.097494217 |
| | | ID NO: 3607) | | | | | | | |
| 658 | D | E | 0.122389699 | 0.080353294 | 676 | P | H | 0.109607681 | 0.091744681 |
| 206 | H | Q | 0.122384978 | 0.08971464 | 484 | KWYG (SEQ | NSSL (SEQ | 0.109535927 | 0.106819917 |
| | | | | | | ID NO: 3273) | ID NO: 3600) | | |
| 689 | H | Q | 0.122256431 | 0.089420446 | 511 | QY | PV | 0.109451554 | 0.106726398 |
| 306 | LN | PV | 0.121921649 | 0.07283705 | 979 | LE[stop]GSP | VSSKDV | 0.108902792 | 0.077647274 |
| | | | | | | | (SEQ ID NO: | | |
| | | | | | | | 3824) | | |
| 620 | LY | PV | 0.121921649 | 0.084823364 | 420 | A | V | 0.108649806 | 0.097722159 |
| 910 | -- | SG | 0.121685511 | 0.114110877 | 53 | N | K | 0.108567111 | 0.086753227 |
| 508 | -------- | FSKQYNCA | 0.121235544 | 0.060533533 | 114 | P | A | 0.108538006 | 0.106859466 |
| | | (SEQ ID NO: | | | | | | | |
| | | 3417) | | | | | | | |
| 314 | I | F | 0.120726616 | 0.074980055 | 637 | ------- | TFERREV | 0.108360722 | 0.063051456 |
| | | | | | | | (SEQ ID NO: | | |
| | | | | | | | 3746) | | |
| 746 | VT | C- | 0.120516649 | 0.087097894 | 286 | TK | DR | 0.108360722 | 0.053025872 |
| 910 | VC | CL | 0.119637812 | 0.085877084 | 249 | EH | AV | 0.108360722 | 0.095653705 |
| 621 | ------ | YNRRTR | 0.119637812 | 0.065553526 | 67 | NK | DR | 0.108360722 | 0.039884349 |
| | | (SEQ ID NO: | | | | | | | |
| | | 3853) | | | | | | | |
| 467 | ------ | LKEAD (SEQ | 0.119637812 | 0.109940477 | 944 | ------- | QTNKTTG | 0.108360722 | 0.078648908 |
| | | ID NO: 3555) | | | | | (SEQ ID NO: | | |
| | | | | | | | 3654) | | |
| 827 | - | KL | 0.119637812 | 0.054530509 | 513 | ------ | NCAFIW | 0.108360722 | 0.045078115 |
| | | | | | | | (SEQ ID NO: | | |
| | | | | | | | 3588) | | |
| 374 | --- | QEA | 0.119637812 | 0.063378708 | 429 | ---- | EGLS (SEQ | 0.108360722 | 0.046808088 |
| | | | | | | | ID NO: 3384) | | |
| 145 | --- | NDK | 0.119637812 | 0.051846935 | 615 | VI | AV | 0.108360722 | 0.089957198 |
| 979 | LE[stop]GSPG | FSSKDLQ | 0.119637812 | 0.067517262 | 927 | ---- | NIAR (SEQ | 0.108360722 | 0.096224338 |
| | (SEQ ID NO: | (SEQ ID NO: | | | | | ID NO: 3593) | | |
| | 3251) | 3419) | | | | | | | |
| 338 | --- | ANE | 0.119637812 | 0.103007188 | 56 | Q | V | 0.108360722 | 0.076115958 |
| 389 | KG | R- | 0.119637812 | 0.050940425 | 852 | YY | C- | 0.108360722 | 0.054744482 |
| 587 | ------ | FGKRQG | 0.118677331 | 0.110043529 | 816 | IT | LS | 0.108360722 | 0.074232993 |
| | | (SEQ ID NO: | | | | | | | |
| | | 3411) | | | | | | | |
| 783 | ------ | TAKLAY | 0.118677331 | 0.076704941 | 210 | P | S | 0.108088041 | 0.085752595 |
| | | (SEQ ID NO: | | | | | | | |
| | | 3736) | | | | | | | |
| 542 | -- | FK | 0.118677331 | 0.098685141 | 251 | --- | QKV | 0.107840626 | 0.092439 |
| 733 | ------ | MVRNTAR | 0.118677331 | 0.078476963 | 351 | ---- | KKLI (SEQ | 0.107840626 | 0.05939446 |
| | | (SEQ ID NO: | | | | | ID NO: 3502) | | |
| | | 3586) | | | | | | | |
| 396 | ---- | YQFG (SEQ | 0.118677331 | 0.08225792 | 962 | ------ | QSFYRKK | 0.107840626 | 0.060903469 |
| | | ID NO: 3855) | | | | | (SEQ ID NO: | | |
| | | | | | | | 3651) | | |
| 837 | ----- | TTING (SEQ | 0.118677331 | 0.059978646 | 594 | EFI | DCL | 0.107840626 | 0.078577001 |
| | | ID NO: 3762) | | | | | | | |
| 729 | L | P | 0.118360335 | 0.091091038 | 600 | --- | LLS | 0.107840626 | 0.107212137 |
| 194 | D | E | 0.117679069 | 0.090466918 | 979 | LE[stop]GS- | ASSKDLQAS | 0.107840626 | 0.073484536 |
| | | | | | | PGIK (SEQ ID | N (SEQ ID | | |
| | | | | | | NO: 3279) | NO: 3333) | | |
| 582 | ILP | SC- | 0.11732562 | 0.090313521 | 606 | --- | GSL | 0.107840626 | 0.104907627 |
| 901 | --- | SHR | 0.11712133 | 0.108439325 | 604 | --- | ETG | 0.107840626 | 0.105428162 |
| 67 | N | D | 0.116939695 | 0.113264127 | 473 | ------- | DEFCRCE | 0.107840626 | 0.072973962 |
| | | | | | | | (SEQ ID NO: | | |
| | | | | | | | 3351) | | |
| 309 | W | R | 0.116671977 | 0.111491729 | 798 | ------ | SKTLAQ | 0.107840626 | 0.085530107 |
| | | | | | | | (SEQ ID NO: | | |
| | | | | | | | 3713) | | |
| 74 | T | S | 0.11653877 | 0.0855649 | 607 | ----- | SLKLA (SEQ | 0.107840626 | 0.087611083 |
| | | | | | | | ID NO: 3178) | | |
| 838 | T | N | 0.116394614 | 0.094955966 | 705 | Q- | ET | 0.107840626 | 0.102652999 |
| 137 | Y | [stop] | 0.116334699 | 0.088258455 | 215 | GG | CL | 0.105199237 | 0.057087854 |
| 591 | Q | [stop] | 0.116290785 | 0.093561727 | 886 | KG | TV | 0.105199237 | 0.077099458 |
| 686 | N | K | 0.116232458 | 0.062605741 | 198 | -I | TV | 0.105199237 | 0.087584827 |
| 445 | ----- | DAQSK (SEQ | 0.115532631 | 0.10378499 | 878 | NN | DS | 0.105199237 | 0.079694461 |
| | | ID NO: 3344) | | | | | | | |
| 134 | Q | P | 0.114967131 | 0.11371497 | 76 | MK | IC | 0.105199237 | 0.090203405 |
| 698 | - | KE | 0.114412847 | 0.098843087 | 227 | ALSDA (SEQ | SPERR (SEQ | 0.105199237 | 0.101107303 |
| | | | | | | ID NO: 3252) | ID NO: 3727) | | |
| 701 | QR | PV | 0.114412847 | 0.104102361 | 134 | Q-P | HCL | 0.105199237 | 0.057452451 |
| 281 | --- | PPQ | 0.114412847 | 0.077542482 | 794 | K-T | NCL | 0.105199237 | 0.055344005 |
| 708 | K | [stop] | 0.113715295 | 0.106986973 | 532 | ----- | INYFK (SEQ | 0.105199237 | 0.091675146 |
| | | | | | | | ID NO: 3478) | | |
| 696 | SYK | LQR | 0.113676993 | 0.07036758 | 558 | VI | AV | 0.105199237 | 0.093989814 |
| 703 | -- | TIQ | 0.113676993 | 0.062517799 | 610 | -- | LA | 0.105199237 | 0.085523633 |
| 596 | I | F | 0.113504467 | 0.107709004 | 82 | -H | DS | 0.105199237 | 0.045790293 |
| 197 | ------ | SIHVTRE | 0.108360722 | 0.081689422 | 780 | DW | AV | 0.105199237 | 0.092887336 |
| | | (SEQ ID NO: | | | | | | | |
| | | 3710) | | | | | | | |
| 510 | KQYNCA | SHLQNS | 0.108360722 | 0.044585998 | 708 | ------------- | KEVEQR | 0.105052225 | 0.060231645 |
| | (SEQ ID NO: | (SEQ ID NO: | | | | | (SEQ ID NO: | | |
| | 3271) | 3706) | | | | | 3493) | | |
| 953 | D | C | 0.108360722 | 0.098828046 | 548 | EAFE (SEQ | RPSR (SEQ | 0.105052225 | 0.087924295 |
| | | | | | | ID NO: 3255) | ID NO: 3675) | | |
| 63 | RA | SC | 0.108360722 | 0.091093584 | 251 | ----- | QKVIK (SEQ | 0.105052225 | 0.044504449 |
| | | | | | | | ID NO: 3642) | | |
| 597 | ----- | WNDLL (SEQ | 0.108360722 | 0.065802495 | | | | | |
| | | ID NO: 3842) | | | 497 | EA | AV | 0.105052225 | 0.084527693 |
| 208 | VK | CL | 0.108360722 | 0.044537036 | 841 | ------- | GKELKVE | 0.105052225 | 0.091417746 |
| | | | | | | | (SEQ ID NO: | | |
| | | | | | | | 3433) | | |
| 468 | ------- | KEADKDE | 0.108360722 | 0.074432186 | 575 | F- | LS | 0.105052225 | 0.076582865 |
| | | (SEQ ID NO: | | | | | | | |
| | | 3491) | | | | | | | |
| 84 | -Y | DS | 0.108360722 | 0.088490546 | 910 | ----- | VCLNC (SEQ | 0.105052225 | 0.090851749 |
| | | | | | | | ID NO: 3769) | | |
| 496 | -- | IE | 0.108360722 | 0.07371372 | 570 | ----- | EVNFN (SEQ | 0.104207678 | 0.100821855 |
| | | | | | | | ID NO: 3407) | | |
| 672 | P---E | SGCV (SEQ | 0.108360722 | 0.07159837 | 661 | -- | EN | 0.104134797 | 0.102286534 |
| | | ID NO: | | | | | | | |
| | | 3701)[stop] | | | | | | | |
| 910 | VC | AV | 0.108360722 | 0.062775349 | 500 | --- | NSI | 0.104134797 | 0.058937244 |
| 868 | EL | DR | 0.108360722 | 0.050620256 | 420 | ------- | AWERIDK | 0.104134797 | 0.06870659 |
| | | | | | | | (SEQ ID NO: | | |
| | | | | | | | 3337) | | |
| 235 | -- | AV | 0.108360722 | 0.094955272 | 285 | ------- | HTKEGIE | 0.10063092 | 0.059060467 |
| | | | | | | | (SEQ ID NO: | | |
| | | | | | | | 3465) | | |
| 332 | PL | RQ | 0.108360722 | 0.062876398 | 347 | --- | VCN | 0.10063092 | 0.070834064 |
| 461 | ------- | SFVIEGLK | 0.108360722 | 0.064022496 | 671 | - | D | 0.10063092 | 0.070617109 |
| | | (SEQ ID NO: | | | | | | | |
| | | 3699) | | | | | | | |
| 562 | KSGEI (SEQ | SPAR (SEQ | 0.108360722 | 0.067954904 | 103 | AP | DS | 0.10063092 | 0.044259819 |
| | ID NO: 3272) | ID NO: 3726)- | | | | | | | |
| 556 | ------ | YTVINKK | 0.108360722 | 0.070852948 | 584 | --- | PLA | 0.10063092 | 0.096095285 |
| | | (SEQ ID NO: | | | | | | | |
| | | 3861) | | | | | | | |
| 121 | RLT | SC- | 0.108360722 | 0.070897115 | 685 | GN | DS | 0.10063092 | 0.057986016 |
| 868 | EL | NW | 0.108360722 | 0.108128749 | 837 | ------- | TTINGKE | 0.10063092 | 0.070942034 |
| | | | | | | | (SEQ ID NO: | | |
| | | | | | | | 3763) | | |
| 745 | ---- | AVTQ (SEQ | 0.108360722 | 0.088762315 | 509 | ---- | SKQY (SEQ | 0.10063092 | 0.078527136 |
| | | ID NO: 3335) | | | | | ID NO: 3711) | | |
| 674 | ------ | GCPLSR | 0.107840626 | 0.089241733 | 914 | -C | LS | 0.10063092 | 0.094652044 |
| | | (SEQ ID NO: | | | | | | | |
| | | 3424) | | | | | | | |
| 185 | ------- | LGKFGQR | 0.107840626 | 0.068363178 | 932 | --- | WLF | 0.10063092 | 0.060195605 |
| | | (SEQ ID NO: | | | | | | | |
| | | 3547) | | | | | | | |
| 344 | WD | LS | 0.107840626 | 0.066070011 | 979 | LE[stop]G | VSRK (SEQ | 0.10063092 | 0.052097814 |
| | | | | | | | ID NO: 3794) | | |
| 274 | - | AF | 0.107840626 | 0.075101467 | 194 | ------ | DFYSIH (SEQ | 0.10063092 | 0.073983623 |
| | | | | | | | ID NO: 3354) | | |
| 577 | D | G | 0.1075508 | 0.10472372 | 596 | ---- | IWND (SEQ | 0.10063092 | 0.075782386 |
| | | | | | | | ID NO: 3486) | | |
| 700 | K | M | 0.107451835 | 0.099853237 | 32 | L | S | 0.099998377 | 0.098160777 |
| 641 | -- | RE | 0.106527066 | 0.104478931 | 822 | D | E | 0.099951571 | 0.083423411 |
| 599 | ---- | DLLS (SEQ | 0.106527066 | 0.100649327 | 957 | F | S | 0.099918571 | 0.054364404 |
| | | ID NO: 3363) | | | | | | | |
| 564 | GE | DR | 0.106527066 | 0.090487961 | 902 | ---- | HRPV (SEQ | 0.099764722 | 0.080515888 |
| | | | | | | | ID NO: 3462) | | |
| 836 | MT | IC | 0.106527066 | 0.100530022 | 474 | ----- | EFCRC (SEQ | 0.099764722 | 0.089224756 |
| | | | | | | | ID NO: 3383) | | |
| 853 | ----- | YNRYK (SEQ | 0.106527066 | 0.088862545 | 242 | --- | KYQ | 0.099764722 | 0.054563676 |
| | | ID NO: 3854) | | | | | | | |
| 586 | ---- | AFGK (SEQ | 0.106527066 | 0.08642655 | 342 | D | C | 0.099764722 | 0.075335971 |
| | | ID NO: 3311) | | | | | | | |
| 275 | -F | SV | 0.106527066 | 0.099879454 | 413 | -- | WG | 0.099764722 | 0.079591734 |
| 429 | -- | EG | 0.106527066 | 0.066947062 | 149 | ------- | KPHTNYF | 0.099764722 | 0.070518497 |
| | | | | | | | (SEQ ID NO: | | |
| | | | | | | | 3522) | | |
| 612 | N | T | 0.106459427 | 0.08415093 | 510 | KQY | SHL | 0.099764722 | 0.087972807 |
| 611 | --- | ANG | 0.105912094 | 0.09807063 | 775 | ---- | YTRM (SEQ | 0.097097924 | 0.054287911 |
| | | | | | | | ID NO: 3857) | | |
| 563 | ----- | SGEIV (SEQ | 0.105912094 | 0.10402865 | 607 | -- | SL | 0.097097924 | 0.071187897 |
| | | ID NO: 3703) | | | | | | | |
| 203 | E- | DR | 0.10545658 | 0.048953383 | 897 | -K | TE | 0.097097924 | 0.05492748 |
| 872 | -- | LS | 0.10545658 | 0.08227801 | 118 | GN | DS | 0.097097924 | 0.083309653 |
| 291 | EA | -C | 0.10545658 | 0.078263499 | 425 | D | V | 0.096834118 | 0.093228512 |
| 894 | S- | TG | 0.10545658 | 0.077864616 | 704 | -- | IQ | 0.096824625 | 0.053400496 |
| 851 | -T | LS | 0.10545658 | 0.071676834 | 207 | ---- | PVKPLE | 0.096824625 | 0.074740089 |
| | | | | | | | (SEQ ID NO: | | |
| | | | | | | | 3630) | | |
| 251 | -- | QK | 0.105199237 | 0.101057895 | 154 | -- | YF | 0.096824625 | 0.067984555 |
| 194 | ----- | DFYSI (SEQ | 0.105199237 | 0.05958457 | 668 | ---- | ALTD (SEQ | 0.096824625 | 0.088221952 |
| | | ID NO: 3353) | | | | | ID NO: 3322) | | |
| 236 | --- | VAS | 0.105199237 | 0.084024149 | 386 | -- | DR | 0.096824625 | 0.067625309 |
| 899 | RF | SC | 0.105199237 | 0.046835281 | 388 | ---- | KKGK (SEQ | 0.096824625 | 0.060426936 |
| | | | | | | | ID NO: 3498) | | |
| 533 | ---- | NYFK (SEQ | 0.104134797 | 0.074535749 | 880 | ---- | DISS (SEQ ID | 0.096824625 | 0.089590245 |
| | | ID NO: 3609) | | | | | NO: 3358) | | |
| 747 | --- | TQD | 0.104134797 | 0.072847901 | 783 | -------- | TAKLAYEG | 0.096824625 | 0.064829377 |
| | | | | | | | (SEQ ID NO: | | |
| | | | | | | | 3737) | | |
| 371 | -- | YK | 0.104134797 | 0.087850723 | 643 | -------- | VLDSSNIK | 0.096824625 | 0.089286037 |
| | | | | | | | (SEQ ID NO: | | |
| | | | | | | | 3785) | | |
| 625 | TR | -Q | 0.104134797 | 0.077810682 | 157 | --- | RCN | 0.096824625 | 0.095145301 |
| 195 | -- | FY | 0.104134797 | 0.074775738 | 576 | ------- | DDPNLII | 0.096824625 | 0.040738988 |
| | | | | | | | (SEQ ID NO: | | |
| | | | | | | | 3346) | | |
| 464 | -- | IE | 0.103802674 | 0.096071807 | 296 | ----- | VVAQI (SEQ | 0.096824625 | 0.081486595 |
| | | | | | | | ID NO: 3836) | | |
| 451 | A | T | 0.103708002 | 0.093659384 | 559 | -I | CL | 0.096824625 | 0.07248553 |
| 245 | DII | ETV | 0.10291048 | 0.070762893 | 979 | LE-[stop] | VSIK (SEQ ID | 0.096824625 | 0.050151323 |
| | | | | | | | NO: 3792) | | |
| 504 | ---- | DISG (SEQ ID | 0.10291048 | 0.066659076 | 767 | ------ | RTFMAE | 0.096824625 | 0.057097889 |
| | | NO: 3356) | | | | | (SEQ ID NO: | | |
| | | | | | | | 3692) | | |
| 323 | -Q | IH | 0.10291048 | 0.071312882 | 820 | ------- | DYDRVLE | 0.091736446 | 0.087280678 |
| | | | | | | | (SEQ ID NO: | | |
| | | | | | | | 3371) | | |
| 638 | ----- | FERRE (SEQ | 0.10291048 | 0.096842919 | 415 | KVY | NC- | 0.091736446 | 0.087802292 |
| | | ID NO: 3409) | | | | | | | |
| 593 | ------- | REFIWNDLL | 0.10291048 | 0.079136445 | 674 | GCPL (SEQ | DAH[stop] | 0.091736446 | 0.089744971 |
| | | (SEQ ID NO: | | | | ID NO: 3260) | | | |
| | | 3663) | | | | | | | |
| 730 | ------ | ADDMVR | 0.10291048 | 0.102673345 | 705 | QA | -C | 0.091736446 | 0.071260814 |
| | | (SEQ ID NO: | | | | | | | |
| | | 3304) | | | | | | | |
| 827 | KL | TV | 0.10291048 | 0.094773598 | 307 | -N | TD | 0.091736446 | 0.071147866 |
| 138 | VY | C- | 0.10291048 | 0.091363063 | 370 | G- | AV | 0.091736446 | 0.051182414 |
| 310 | QK | DR | 0.10291048 | 0.068590108 | 954 | KRA | T-V | 0.091736446 | 0.081861067 |
| 524 | KKL | RN [stop] | 0.102360708 | 0.063041226 | 326 | KGFPS (SEQ | RASLA (SEQ | 0.091644836 | 0.054125593 |
| | | | | | | ID NO: 3267) | ID NO: 3657) | | |
| 940 | ----- | YKKYQ (SEQ | 0.102324952 | 0.078047936 | 289 | GI | LS | 0.091644836 | 0.069499341 |
| | | ID NO: 3850) | | | | | | | |
| 918 | --- | THA | 0.102324952 | 0.066375654 | 142 | -E | CL | 0.091644836 | 0.064151435 |
| 979 | LE[stop]GSPG | VSSNDLQ | 0.102324952 | 0.073267994 | 10 | RR | TG | 0.091644836 | 0.090788699 |
| | (SEQ ID NO: | (SEQ ID NO: | | | | | | | |
| | 3251) | 3831) | | | | | | | |
| 4 | K | Q | 0.101594625 | 0.098660596 | 193 | LDFYSIH | RTSTAST | 0.091277438 | 0.058446074 |
| | | | | | | (SEQ ID NO: | (SEQ ID NO: | | |
| | | | | | | 3276) | 3694) | | |
| 589 | ----- | KRQGR (SEQ | 0.101233118 | 0.096410486 | 979 | LE[stop]GS- | VSIKDLQAS | 0.091277438 | 0.055852497 |
| | | ID NO: 3529) | | | | PGIK (SEQ ID | NK (SEQ ID | | |
| | | | | | | NO: | NO: 3793) | | |
| | | | | | | 3279)[stop] | | | |
| 211 | ----- | LEQIG (SEQ | 0.101233118 | 0.097193308 | 590 | ----- | RQGRE (SEQ | 0.091277438 | 0.07404543 |
| | | ID NO: 3544) | | | | | ID NO: 3678) | | |
| 649 | I | N | 0.101148579 | 0.091521137 | 308 | --- | LWQ | 0.091277438 | 0.063930973 |
| 220 | ------ | ASGPVG | 0.099764722 | 0.05025267 | 311 | -------- | KLKIGRDEA | 0.091277438 | 0.090951045 |
| | | (SEQ ID NO: | | | | | (SEQ ID NO: | | |
| | | 3330) | | | | | 3509) | | |
| 787 | AYEG (SEQ | PTRD (SEQ | 0.099764722 | 0.069079749 | 585 | ------ | LAFGKR | 0.091277438 | 0.057801256 |
| | ID NO: 3253) | ID NO: 3629) | | | | | (SEQ ID NO: | | |
| | | | | | | | 3534) | | |
| 888 | ----- | RSGEA (SEQ | 0.099764722 | 0.094243718 | 466 | ------- | GLKEADK | 0.091277438 | 0.064806465 |
| | | ID NO: 3685) | | | | | (SEQ ID NO: | | |
| | | | | | | | 3443) | | |
| 504 | ------ | DISGFS (SEQ | 0.099764722 | 0.091750112 | 414 | -- | GK | 0.089604136 | 0.067494445 |
| | | ID NO: 3357) | | | | | | | |
| 323 | QR | RD | 0.099764722 | 0.040967673 | 979 | LE[stop]GSPG | ISSKDLQ | 0.089062173 | 0.071078934 |
| | | | | | | (SEQ ID NO: | (SEQ ID NO: | | |
| | | | | | | 3251) | 3482) | | |
| 647 | SN | DS | 0.099764722 | 0.071118435 | 300 | ---- | IVIW (SEQ ID | 0.089062173 | 0.052509601 |
| | | | | | | | NO: 3485) | | |
| 740 | DLLY (SEQ | SAV- | 0.099753827 | 0.050146089 | 209 | KP | TV | 0.089062173 | 0.046404323 |
| | ID NO: 3254) | | | | | | | | |
| 38 | - | A | 0.099114744 | 0.090540757 | 851 | -T | CL | 0.089062173 | 0.047830666 |
| 261 | LA | PV | 0.099083678 | 0.060781559 | 466 | GL | LS | 0.089062173 | 0.060367604 |
| 255 | ---- | KKNE (SEQ | 0.098543421 | 0.07624083 | 202 | RE-- | SSSL (SEQ ID | 0.089062173 | 0.059904595 |
| | | ID NO: 3505) | | | | | NO: 3730) | | |
| 280 | ---- | LPPQ (SEQ | 0.098543421 | 0.069822078 | 291 | EA | DC | 0.089062173 | 0.078319771 |
| | | ID NO: 3567) | | | | | | | |
| 308 | LW | PV | 0.097993366 | 0.087176639 | 871 | RL | LS | 0.089062173 | 0.055570451 |
| 753 | --- | IFA | 0.097806547 | 0.045793305 | 874 | EE | DR | 0.089062173 | 0.077193595 |
| 205 | N | I | 0.097706358 | 0.075812724 | 868 | ELDR (SEQ | NWT- | 0.089062173 | 0.059312334 |
| | | | | | | ID NO: 3257) | | | |
| 142 | E | Q | 0.097553503 | 0.074603349 | 301 | VI | AV | 0.089062173 | 0.083633904 |
| 717 | ------- | GYSRKYAS | 0.097097924 | 0.054767341 | 208 | ---- | VKPLEQI | 0.089062173 | 0.046334388 |
| | | (SEQ ID NO: | | | | | (SEQ ID NO: | | |
| | | 3459) | | | | | 3784) | | |
| 979 | LE[stop]GSPG | VSSKDLH | 0.097097924 | 0.068112769 | 305 | -N | TT | 0.089062173 | 0.072049193 |
| | (SEQ ID NO: | (SEQ ID NO: | | | | | | | |
| | 3251) | 3806) | | | | | | | |
| 527 | NLYL (SEQ | TCT[stop] | 0.097097924 | 0.089930288 | 978 | [stop]L | GP | 0.089062173 | 0.071277586 |
| | ID NO: 3283) | | | | | | | | |
| 230 | D | T | 0.097097924 | 0.061172404 | 866 | S- | TG | 0.089062173 | 0.056446779 |
| 595 | ---- | FIWN (SEQ | 0.097097924 | 0.075559339 | 628 | DE | LS | 0.089062173 | 0.070268313 |
| | | ID NO: 3413) | | | | | | | |
| 526 | LN | PV | 0.097097924 | 0.065035268 | 651 | -P | TA | 0.089062173 | 0.05500823 |
| 928 | IA | TV | 0.096824625 | 0.059262285 | 276 | --- | PKI | 0.089062173 | 0.06318371 |
| 694 | --- | GES | 0.096824625 | 0.04858003 | 299 | - | V | 0.089062173 | 0.08531757 |
| 190 | --- | QRA | 0.096824625 | 0.080026424 | 346 | -- | MV | 0.089062173 | 0.060831249 |
| 601 | ------- | LSLETGS | 0.096824625 | 0.078527715 | 742 | LY | PV | 0.089062173 | 0.087665343 |
| | | (SEQ ID NO: | | | | | | | |
| | | 3576) | | | | | | | |
| 150 | -- | PH | 0.096482996 | 0.069152449 | 743 | YY | ET | 0.089062173 | 0.059923968 |
| 307 | --- | NLW | 0.096482996 | 0.053647152 | 751 | ML | RQ | 0.089062173 | 0.045208162 |
| 808 | --- | TCS | 0.096381808 | 0.086676449 | 894 | -S | RQ | 0.089062173 | 0.071980752 |
| 687 | ------- | PTHILRI | 0.095815136 | 0.067505643 | 433 | KH | TV | 0.089062173 | 0.061328218 |
| | | (SEQ ID NO: | | | | | | | |
| | | 3628) | | | | | | | |
| 469 | --- | EAD | 0.095416799 | 0.081758814 | 899 | RF | LS | 0.089062173 | 0.083069213 |
| 181 | VTYS (SEQ | SHTA (SEQ | 0.095412022 | 0.081952005 | 582 | --- | ILP | 0.089062173 | 0.053169618 |
| | ID NO: 3295) | ID NO: 3708) | | | | | | | |
| 814 | F | C | 0.095092296 | 0.090308339 | 979 | LE[stop]GS- | VSSKDLHAS | 0.087252372 | 0.071793737 |
| | | | | | | PGIK (SEQ ID | N (SEQ ID | | |
| | | | | | | NO:) | NO: 3807) | | |
| 389 | K | [stop] | 0.094408724 | 0.074513611 | 735 | ------ | RNTARD | 0.087252372 | 0.052948743 |
| | | | | | | | (SEQ ID NO: | | |
| | | | | | | | 3672) | | |
| 663 | I | C | 0.094255793 | 0.075689829 | 227 | ------------ | ALSDACM | 0.087252372 | 0.073258454 |
| | | | | | | | (SEQ ID NO: | | |
| | | | | | | | 3321) | | |
| 979 | L | I | 0.092483102 | 0.077877212 | 151 | HTNYFGRCN | TPTTSADAT | 0.087252372 | 0.05854259 |
| | | | | | | V (SEQ ID | C (SEQ ID | | |
| | | | | | | NO: 3264) | NO: 3758) | | |
| 290 | I- | LS | 0.092483102 | 0.055600721 | 875 | ------ | ESVNND | 0.087252372 | 0.069839022 |
| | | | | | | | (SEQ ID NO: | | |
| | | | | | | | 3397) | | |
| 202 | R-------E | SSSLASGL | 0.092483102 | 0.051559995 | 151 | -H | CL | 0.087252372 | 0.072166234 |
| | | (SEQ ID NO: | | | | | | | |
| | | 3731)[stop] | | | | | | | |
| 130 | S | I | 0.092259428 | 0.091849472 | 517 | ----- | IWQKD (SEQ | 0.087252372 | 0.059389612 |
| | | | | | | | ID NO: 3488) | | |
| 237 | A | V | 0.092157582 | 0.073154252 | 294 | NN | ET | 0.087252372 | 0.054113615 |
| 550 | F- | LS | 0.091736446 | 0.078399586 | 979 | LE[stop]GS- | VSSEDLQAS | 0.087252372 | 0.053550045 |
| | | | | | | PGIK (SEQ ID | NK (SEQ ID | | |
| | | | | | | NO: | NO: 3796) | | |
| | | | | | | 3279)[stop] | | | |
| 352 | --- | KLI | 0.091736446 | 0.062601185 | 280 | LP | C- | 0.087252372 | 0.046361662 |
| 257 | ------ | NEKRLA | 0.091736446 | 0.074344692 | 973 | WK | CL | 0.087252372 | 0.043130788 |
| | | (SEQ ID NO: | | | | | | | |
| | | 3591) | | | | | | | |
| 978 | [stop]LE | QVS | 0.091736446 | 0.070305933 | 859 | - | Q | 0.087252372 | 0.049734005 |
| 878 | NN | ET | 0.091736446 | 0.057372719 | 383 | ----- | SEEDR (SEQ | 0.087252372 | 0.079531899 |
| | | | | | | | ID NO: 3695) | | |
| 484 | -KWYGD | NSSLSA | 0.091736446 | 0.051261975 | 193 | -------- | LDFYSIHVT | 0.087252372 | 0.075700876 |
| | (SEQ ID NO: | (SEQ ID NO: | | | | | (SEQ ID NO: | | |
| | 3274) | 3601) | | | | | 3542) | | |
| 796 | -- | YL | 0.08954136 | 0.077067905 | 731 | ---- | DDMV (SEQ | 0.087252372 | 0.055852115 |
| | | | | | | | ID NO: 3345) | | |
| 872 | --- | LSE | 0.089427419 | 0.072631533 | 586 | --- | AFG | 0.087252372 | 0.059593552 |
| 388 | ----- | KKGKK (SEQ | 0.089427419 | 0.050485092 | 11 | RR | GD | 0.087252372 | 0.07840862 |
| | | ID NO: 3499) | | | | | | | |
| 211 | LEQIGG | RNRSAA | 0.089427419 | 0.058037112 | 979 | LE[stop]G | VPSK (SEQ | 0.086010969 | 0.05573546 |
| | (SEQ ID NO: | (SEQ ID NO: | | | | | ID NO: 3787) | | |
| | 3281) | 3671) | | | | | | | |
| 193 | LDFYSIHV | RTSTAST | 0.089427419 | 0.06189365 | 671 | D | V | 0.084756133 | 0.072837893 |
| | (SEQ ID NO: | (SEQ ID NO: | | | | | | | |
| | 3277) | 3694)[stop] | | | | | | | |
| 769 | FMAERQY | LWPRGST | 0.089427419 | 0.048645432 | 462 | --- | FVI | 0.083590457 | 0.068208408 |
| | (SEQ ID NO: | (SEQ ID NO: | | | | | | | |
| | 3258) | 3582) | | | | | | | |
| 558 | --- | VIN | 0.089427419 | 0.08506841 | 619 | TLYNRRTR | PCTTGEPD | 0.083590457 | 0.071170573 |
| | | | | | | (SEQ ID NO: | (SEQ ID NO: | | |
| | | | | | | 3292) | 3613) | | |
| 973 | --- | WKP | 0.089427419 | 0.059845159 | 337 | QA | PV | 0.083590457 | 0.078536227 |
| 285 | ---- | HTKE (SEQ | 0.089427419 | 0.058488636 | 418 | ---- | DEAW (SEQ | 0.083590457 | 0.038813523 |
| | | ID NO: 3463) | | | | | ID NO: 3347) | | |
| 353 | -- | LI | 0.089427419 | 0.055053978 | 426 | -- | KK | 0.083590457 | 0.07413354 |
| 950 | ---- | GNTD (SEQ | 0.089427419 | 0.068410765 | 208 | VK | AV | 0.083590457 | 0.037512118 |
| | | ID NO: 3445) | | | | | | | |
| 642 | ----- | EVLDS (SEQ | 0.089427352 | 0.04064403 | 519 | -- | QK | 0.083590457 | 0.082570582 |
| | | ID NO: 3405) | | | | | | | |
| 586 | AF | ET | 0.089427352 | 0.026351335 | 122 | LT | D[stop] | 0.083590457 | 0.076976074 |
| 147 | KG | C- | 0.089427352 | 0.03353623 | 659 | RG | PV | 0.083590457 | 0.0659041 |
| 473 | ----- | DEFCR (SEQ | 0.089427352 | 0.087380064 | 160 | ------- | VSEHERL | 0.083590457 | 0.081613302 |
| | | ID NO: 3350) | | | | | (SEQ ID NO: | | |
| | | | | | | | 3790) | | |
| 62 | SR | CL | 0.089427352 | 0.085389222 | 278 | IT | TA | 0.083590457 | 0.047460329 |
| 946 | N | C | 0.089427352 | 0.086906423 | 242 | KY | CL | 0.083590457 | 0.045794039 |
| 341 | ----- | VDWWD | 0.089427352 | 0.088291312 | 518 | WQ | GR | 0.08340916 | 0.072293259 |
| | | (SEQ ID NO: | | | | | | | |
| | | 3772) | | | | | | | |
| 546 | --- | KPE | 0.089427352 | 0.070048864 | 513 | ---- | NCAF (SEQ | 0.08340916 | 0.058923148 |
| | | | | | | | ID NO: 3587) | | |
| 979 | LE[stop]G-- | VSSKDLQAC | 0.089062173 | 0.059857989 | 31 | L | C | 0.082126328 | 0.081561344 |
| | SPGI (SEQ ID | L (SEQ ID | | | | | | | |
| | NO: 3278) | NO: 3811) | | | | | | | |
| 944 | --- | QTN | 0.089062173 | 0.066135158 | 868 | E | G | 0.081974564 | 0.070868354 |
| 170 | SP | RQ | 0.089062173 | 0.059574685 | | | | | |
| 771 | ----- | AERQY (SEQ | 0.089062173 | 0.079594468 | 681 | ----- | KDSLG (SEQ | 0.080796062 | 0.070617083 |
| | | ID NO: 3309) | | | | | ID NO: 3489) | | |
| 808 | TC | DS | 0.089062173 | 0.069853908 | 552 | -- | AN | 0.080796062 | 0.080329675 |
| 347 | -- | VC | 0.089062173 | 0.085265549 | 168 | --- | LLS | 0.080796062 | 0.076933587 |
| 554 | RF | SC | 0.089062173 | 0.05713278 | 418 | -------- | DEAWERID | 0.080796062 | 0.062400841 |
| | | | | | | | (SEQ ID NO: | | |
| | | | | | | | 3349) | | |
| 419 | EA | LS | 0.089062173 | 0.062902243 | 356 | ----- | EKKED (SEQ | 0.080428937 | 0.076250147 |
| | | | | | | | ID NO: 3391) | | |
| 184 | ------ | SLGKFG | 0.089062173 | 0.066443269 | 904 | -- | PV | 0.077521024 | 0.061782081 |
| | | (SEQ ID NO: | | | | | | | |
| | | 3716) | | | | | | | |
| 524 | K-K | ETE | 0.089062173 | 0.078642197 | 8 | KIR | ETG | 0.075979618 | 0.06718831 |
| 544 | KI | NC | 0.089062173 | 0.051439626 | 963 | ---- | SFYR (SEQ | 0.075979618 | 0.064323698 |
| | | | | | | | ID NO: 3700 | | |
| 417 | ------ | YDEAWE | 0.089062173 | 0.084599468 | 34 | RV | SC | 0.075979618 | 0.063118319 |
| | | (SEQ ID NO: | | | | | | | |
| | | 3847) | | | | | | | |
| 911 | CL | DR | 0.089062173 | 0.07167912 | 369 | ------ | AGYKRQ | 0.075979618 | 0.050848396 |
| | | | | | | | (SEQ ID NO: | | |
| | | | | | | | 3313) | | |
| 735 | -------- | RNTARDLLY | 0.089062173 | 0.058412514 | 242 | KY | TV | 0.075979618 | 0.056127246 |
| | | (SEQ ID NO: | | | | | | | |
| | | 3673) | | | | | | | |
| 305 | N | D | 0.089057834 | 0.075458081 | 297 | VAQIV (SEQ | WPRS (SEQ | 0.075979618 | 0.07433917 |
| | | | | | | ID NO: 3293) | ID NO: | | |
| | | | | | | | 3843)[stop] | | |
| 886 | KGR | RAD | 0.08869535 | 0.056741957 | 672 | -P | LS | 0.075979618 | 0.056690099 |
| 235 | A | P | 0.088591922 | 0.085721293 | 650 | KP | TV | 0.075979618 | 0.062837656 |
| 494 | ------- | FAIEAEN | 0.088487772 | 0.046582849 | 454 | DW | AV | 0.075979618 | 0.049282705 |
| | | (SEQ ID NO: | | | | | | | |
| | | 3408) | | | | | | | |
| 957 | F | Y | 0.088355066 | 0.088244344 | 312 | LK | PV | 0.075979618 | 0.074673373 |
| 670 | ----- | TDPEG (SEQ | 0.087352311 | 0.070989739 | 636 | LT | PV | 0.075651042 | 0.051037357 |
| | | ID NO: 3742) | | | | | | | |
| 388 | -- | KK | 0.087352311 | 0.077174067 | 325 | ----- | LKGFP (SEQ | 0.075651042 | 0.068819815 |
| | | | | | | | ID NO: 3557) | | |
| 294 | -- | NN | 0.087352311 | 0.079627552 | 669 | L | E | 0.075651042 | 0.075396635 |
| 748 | ------ | QDAMLI | 0.087352311 | 0.070738039 | 79 | A | V | 0.074780904 | 0.074608034 |
| | | (SEQ ID NO: | | | | | | | |
| | | 3632) | | | | | | | |
| 978 | [stop]LE[stop] | SVSSK (SEQ | 0.087252372 | 0.078631278 | 887 | | GRSGEA | 0.073542892 | 0.072424639 |
| | G | ID NO: 3734) | | | | | (SEQ ID NO: | | |
| | | | | | | | 3452) | | |
| 743 | ------ | YYAVTQ | 0.087252372 | 0.074424467 | 404 | EIL | DR | 0.073542892 | 0.054184233 |
| | | (SEQ ID NO: | | | | | | | |
| | | 3865) | | | | | | | |
| 90 | KDP | NCL | 0.087252372 | 0.062483354 | 190 | Q-R | HVA | 0.073542892 | 0.04828771 |
| 459 | --- | KAS | 0.087252372 | 0.077679223 | 811 | NC | DS | 0.073542892 | 0.073088889 |
| 319 | -------- | AKPLQRLK | 0.087252372 | 0.077741662 | 824 | ---- | VLEK (SEQ | 0.073542892 | 0.055393108 |
| | | (SEQ ID NO: | | | | | ID NO: 3786) | | |
| | | 3316) | | | | | | | |
| 844 | ------- | LKVEGQI | 0.087252372 | 0.078010123 | 63 | RA | TV | 0.073542892 | 0.069467367 |
| | | (SEQ ID NO: | | | | | | | |
| | | 3558) | | | | | | | |
| 964 | ----- | FYRKK (SEQ | 0.087252372 | 0.061717189 | 350 | VK | AV | 0.072378636 | 0.048322939 |
| | | ID NO: 3422) | | | | | | | |
| 510 | ----- | KQYNC (SEQ | 0.087252372 | 0.072460113 | 690 | ILRI (SEQ ID | PEN- | 0.072378636 | 0.05860973 |
| | | ID NO: 3526) | | | | NO: 3265) | | | |
| 211 | LE | C- | 0.087252372 | 0.072615166 | 384 | EED | D-C | 0.072378636 | 0.064425519 |
| 154 | --- | YFG | 0.087252372 | 0.050562832 | 349 | ------- | NVKKLIN | 0.071251281 | 0.055420168 |
| | | | | | | | (SEQ ID NO: | | |
| | | | | | | | 3605) | | |
| 428 | - | V | 0.087252372 | 0.070602271 | 427 | KVE | NCL | 0.071251281 | 0.037488341 |
| 328 | ------- | FPSFPLV | 0.087252372 | 0.050986167 | 537 | GGKLRFK | AASCGSR | 0.071251281 | 0.047685675 |
| | | (SEQ ID NO: | | | | (SEQ ID NO: | (SEQ ID NO: | | |
| | | 3415) | | | | 3261) | 3301) | | |
| 334 | --- | VER | 0.087252372 | 0.083245674 | 486 | ----- | YGDLR (SEQ | 0.071251281 | 0.057530417 |
| | | | | | | | ID NO: 3849) | | |
| 635 | --- | ALT | 0.087252372 | 0.058640453 | 586 | ------- | AFGKRQG | 0.071251281 | 0.055531439 |
| | | | | | | | (SEQ ID NO: | | |
| | | | | | | | 3312) | | |
| 87 | EF | DC | 0.087252372 | 0.084662756 | 850 | ---- | ITYY (SEQ | 0.071251281 | 0.070061657 |
| | | | | | | | ID NO: 34843) | | |
| 763 | ---- | RQGK (SEQ | 0.087252372 | 0.06272177 | 929 | --- | ARS | 0.071251281 | 0.070844259 |
| | | ID NO: 3677) | | | | | | | |
| 525 | ---- | KLNL (SEQ | 0.087252372 | 0.087055601 | 617 | EK | AV | 0.071251281 | 0.056273969 |
| | | ID NO: 3511) | | | | | | | |
| 482 | LQK | PLM | 0.087252372 | 0.0864173 | 977 | V[stop] | AV | 0.071036023 | 0.057250091 |
| 228 | -- | LS | 0.087252372 | 0.071648918 | 522 | --- | GVK | 0.071036023 | 0.066325629 |
| 149 | ---- | KPHT (SEQ | 0.087252372 | 0.063809398 | 903 | RP | LS | 0.070891186 | 0.042147704 |
| | | ID NO: 3520) | | | | | | | |
| 14 | VKDSNTK | SRTATQR | 0.087252372 | 0.086609324 | 689 | HI | P- | 0.070270828 | 0.063050321 |
| | (SEQ ID NO: | (SEQ ID NO: | | | | | | | |
| | 3294) | 3729) | | | | | | | |
| 567 | VP | C- | 0.087252372 | 0.05902513 | 663 | - | I | 0.070270828 | 0.06150934 |
| 275 | -- | FP | 0.080428937 | 0.059363481 | 649 | IK | RQ | 0.070270828 | 0.060647973 |
| 308 | ------ | LWQKLK | 0.080428937 | 0.078547724 | 258 | -- | EK | 0.070270828 | 0.058125711 |
| | | (SEQ ID NO: | | | | | | | |
| | | 3583) | | | | | | | |
| 15 | KDSNTKK | RTATQRR | 0.080428937 | 0.072523813 | 152 | TN | DS | 0.070270828 | 0.059660679 |
| | (SEQ ID NO: | (SEQ ID NO: | | | | | | | |
| | 3266) | 3690) | | | | | | | |
| 979 | LE[stop]GSPG | VSSKDLQG | 0.080428937 | 0.070440346 | 351 | ----- | KKLINE | 0.070270828 | 0.061736597 |
| | I (SEQ ID NO: | (SEQ ID NO: | | | | | (SEQ ID NO: | | |
| | 3278) | 3818) | | | | | 3503) | | |
| 425 | --- | DKK | 0.080428937 | 0.056582403 | 763 | -- | RQ | 0.070270828 | 0.05541295 |
| 288 | EGI | RAS | 0.080428937 | 0.054809688 | 666 | VI | DS | 0.070270828 | 0.069953364 |
| 849 | QI | R- | 0.080428937 | 0.058314054 | 186 | GK | RQ | 0.066783091 | 0.059043838 |
| 526 | ----- | LNLYL (SEQ | 0.080428937 | 0.073029285 | 242 | ------- | KYQDHLE | 0.066783091 | 0.058248788 |
| | | ID NO: 3564) | | | | | (SEQ ID NO: | | |
| | | | | | | | 3533) | | |
| 546 | ---- | KPEA (SEQ | 0.080428937 | 0.06983999 | 190 | ------- | QRALDFYS | 0.066783091 | 0.060436783 |
| | | ID NO: 3519) | | | | | | | |
| 792 | -- | PS | 0.080428937 | 0.067496853 | 484 | --KWYGDL | NSSLSASF | 0.061911903 | 0.060235262 |
| | | | | | | (SEQ ID NO: | (SEQ ID NO: | | |
| | | | | | | 3275) | 3603) | | |
| 706 | -------- | AAKEVEQR | 0.080428937 | 0.075434091 | 416 | VY | CT | 0.061911903 | 0.058375882 |
| | | (SEQ ID NO: | | | | | | | |
| | | 3300) | | | | | | | |
| 710 | ---- | VEQR (SEQ | 0.080165897 | 0.064037522 | 900 | FS | SV | 0.060850202 | 0.045333847 |
| | | ID NO: 3775) | | | | | | | |
| 949 | -T | LS | 0.080165897 | 0.057028434 | 550 | FE | CL | 0.060850202 | 0.050669807 |
| 224 | V | C | 0.080165897 | 0.062705318 | 169 | LS | -P | 0.059253838 | 0.055169203 |
| 202 | ----- | RESNH (SEQ | 0.08002463 | 0.069004172 | 487 | GD | CL | 0.058561444 | 0.050771143 |
| | | ID NO: 3664) | | | | | | | |
| 380 | YLS | -T[stop] | 0.079267535 | 0.078743084 | 800 | ------ | TLAQYT | 0.058239485 | 0.054115265 |
| | | | | | | | (SEQ ID NO: | | |
| | | | | | | | 3753) | | |
| 617 | --- | EKT | 0.079267535 | 0.066283102 | 863 | KD | RI | 0.058239485 | 0.041340026 |
| 237 | AS | TA | 0.079267535 | 0.061120875 | 407 | KKHGE (SEQ | RSTAR (SEQ | 0.058239485 | 0.049050481 |
| | | | | | | ID NO: 3268) | ID NO: 3687) | | |
| 416 | VYD | C-T | 0.07889536 | 0.067603097 | 593 | ------ | REFIW (SEQ | 0.058239485 | 0.057097188 |
| | | | | | | | ID NO: 3662) | | |
| 554 | -------- | RFYTVINKK | 0.078495111 | 0.06923226 | 979 | LE[stop]G-SP | VSSKVLQ | 0.050653241 | 0.049828056 |
| | | (SEQ ID NO: | | | | | (SEQ ID NO: | | |
| | | 3667) | | | | | 3827) | | |
| 619 | TLYN (SEQ | PC-T | 0.078181072 | 0.043873495 | 42 | ER | A- | 0.050653241 | 0.043693463 |
| | ID NO: 3291) | | | | | | | | |
| 487 | ------ | GDLRGKP | 0.072378636 | 0.071208648 | 897 | -- | KK | 0.050653241 | 0.046680114 |
| | | (SEQ ID NO: | | | | | | | |
| | | 3429) | | | | | | | |
| 644 | L | [stop] | 0.072378636 | 0.060246346 | 294 | NN | DS | 0.049177787 | 0.048944158 |
| 544 | KI | TV | 0.072378636 | 0.05442277 | 186 | GKFGQRAL | ASSDREPWT | 0.049177787 | 0.048777834 |
| | | | | | | DFY (SEQ ID | ST (SEQ ID | | |
| | | | | | | NO: 3262) | NO: | 3331) | |
| 933 | ---- | LFLR (SEQ | 0.072378636 | 0.06374014 | 696 | SYK | -LQ | 0.049177787 | 0.048584657 |
| | | ID NO: 3546) | | | | | | | |
| 276 | PKITLP (SEQ | LRSPCL | 0.072378636 | 0.070970251 | 552 | AN | DS | 0.049177787 | 0.044744659 |
| | ID NO: 3284) | (SEQ ID NO: | | | | | | | |
| | | 3570) | | | | | | | |
| 808 | ------- | TCSNCGFT | 0.072378636 | 0.065622369 | 979 | LE[stop]G- | VSSKYLQAS | 0.049086177 | 0.048688856 |
| | | (SEQ ID NO: | | | | SPGIK (SEQ | NK (SEQ ID | | |
| | | 3740) | | | | ID NO: | NO: 3828) | | |
| | | | | | | 3279)[stop] | | | |
| 978 | [stop]LE[stop] | YVSSKDL | 0.072378636 | 0.066035046 | 413 | -------- | WGKVYDEA | 0.048681821 | 0.046101055 |
| | GS- | (SEQ ID NO: | | | | | (SEQ ID NO: | | |
| | | 3862) | | | | | 3840) | | |
| 919 | HA | PV | 0.072378636 | 0.058676376 | 920 | ----- | AAEQA (SEQ | 0.048224673 | 0.046055533 |
| | | | | | | | ID NO: 3299) | | |
| 378 | -------- | LPYLSSE | 0.072378636 | 0.071574474 | | | | | |
| | | (SEQ ID NO: | | | | | | | |
| | | 3569) | | | | | | | |
| 858 | RQ | LS | 0.072378636 | 0.04290216 | | | | | |
| 152 | -------- | TNYFGRCN | 0.072378636 | 0.054244402 | | | | | |
| | | (SEQ ID NO: | | | | | | | |
| | | 3757) | | | | | | | |
| 859 | ------ | QNVVKD | 0.072378636 | 0.069366552 | | | | | |
| | | (SEQ ID NO: | | | | | | | |
| | | 3644) | | | | | | | |
| 226 | KA | LS | 0.071324732 | 0.06748566 | | | | | |
| 849 | ------ | QITYYN | 0.071251281 | 0.061753986 | | | | | |
| | | (SEQ ID NO: | | | | | | | |
| | | 3640) | | | | | | | |
| 376 | ---- | ALLP (SEQ | 0.071251281 | 0.046839434 | | | | | |
| | | ID NO: 3318) | | | | | | | |
| 660 | --- | GEN | 0.071251281 | 0.063597301 | | | | | |
| | | (SEQ ID NO: | | | | | | | |
| | | 3647) | | | | | | | |
| 615 | VI | DS | 0.066783091 | 0.065544343 | | | | | |
| 295 | | NVVAQI | 0.066783091 | 0.066726619 | | | | | |
| | | (SEQ ID NO: | | | | | | | |
| | | 3608) | | | | | | | |
| 549 | AFE | PTR | 0.066783091 | 0.063274062 | | | | | |
| 924 | -AL | PSG | 0.066783091 | 0.057049314 | | | | | |
| 979 | LE[stop] | VSR | 0.06547263 | 0.059545386 | | | | | |
| 284 | P | L | 0.06489326 | 0.063807972 | | | | | |
| 620 | -- | LY | 0.06268489 | 0.052769076 | | | | | |
| 668 | -A | LS | 0.06268489 | 0.057930418 | | | | | |
| 651 | ---- | PMNL (SEQ | 0.06268489 | 0.054376534 | | | | | |
| | | ID NO: 3619) | | | | | | | |
| 723 | --SK | PPLL (SEQ ID | 0.061911903 | 0.057719078 | | | | | |
| | | NO: 3621) | | | | | | | |
| 788 | YEG | TRD | 0.061911903 | 0.061258021 | | | | | |
| 572 | NF | DS | 0.061911903 | 0.059419672 | | | | | |
| 943 | ---- | YQTN (SEQ | 0.061911903 | 0.05179175 | | | | | |
| | | ID NO: 3856) | | | | | | | |
| 979 | LE[stop]GS-P | VSSKDVQ | 0.061911903 | 0.05324798 | | | | | |
| | | (SEQ ID NO: | | | | | | | |
| | | 3825) | | | | | | | |
| 49 | KK | RS | 0.061911903 | 0.057783548 | | | | | |
| 745 | -A | LS | 0.061911903 | 0.055420231 | | | | | |
| 262 | -AN | ETD | 0.061911903 | 0.056977155 | | | | | |
| 726 | ---- | AKNL (SEQ | 0.061911903 | 0.05965082 | | | | | |
| | | ID NO: 3315) | | | | | | | |
| 583 | ---- | LPLA (SEQ | 0.061911903 | 0.053222838 | | | | | |
| | | ID NO: 3566) | | | | | | | |
| 585 | -- | LA | 0.061911903 | 0.047677961 | | | | | |
| 347 | -------- | VCNVKKLI | 0.061911903 | 0.060561898 | | | | | |
| | | (SEQ ID NO: | | | | | | | |
| | | 3771) | | | | | | | |
| 735 | RN | Q- | 0.061911903 | 0.057911259 | | | | | |
| 176 | AN | TD | 0.061911903 | 0.042711394 | | | | | |
| 979 | LE[stop]GSPG | VSSKDFQ | 0.047884408 | 0.043419619 | | | | | |
| | (SEQ ID NO: | (SEQ | ID NO: | | | | | | |
| | 3251) | 3801) | | | | | | | |
| 423 | RIDKKV | ---NRQ | 0.046868759 | 0.045505043 | | | | | |
| | (SEQ ID NO: | | | | | | | | |
| | 3286) | | | | | | | | |
| 162 | EH | AV | 0.043166861 | 0.040108447 | | | | | |
| 741 | LLY | CC- | 0.041101883 | 0.039741701 | | | | | |
| 443 | SEDAQS | RGRPI (SEQ | 0.041101883 | 0.03770041 | | | | | |
| | (SEQ ID NO: | ID NO: | | | | | | | |
| | 3288) | 3668)[stop] | | | | | | | |
| 767 | RT | TA | 0.041101883 | 0.040956261 | | | | | |

[stop] represent a stop codon, so that amino acids that follow are additional amino acids after a stop codon. (−) holds the position for the insertion shown in the adjacent “Alteration” column. Pos.: Position; Ref.: Reference; Alt.: Alternation; Med. Enrich.: Median Enrichment.

### Example 5: Cleavage Activity of Selected CasX Variant Proteins and Variant Protein:sgRNA Pairs

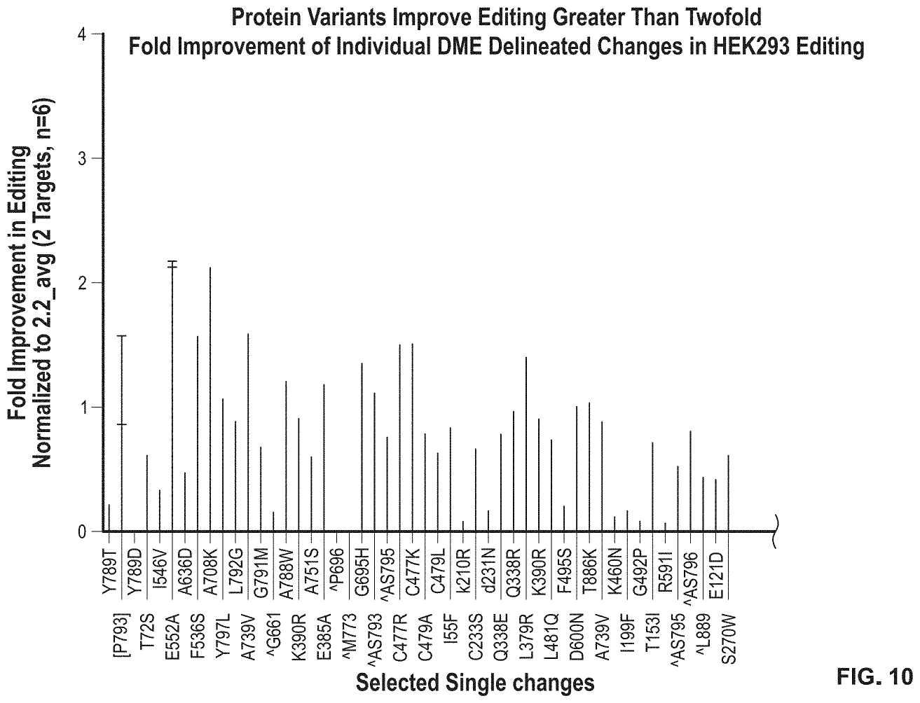

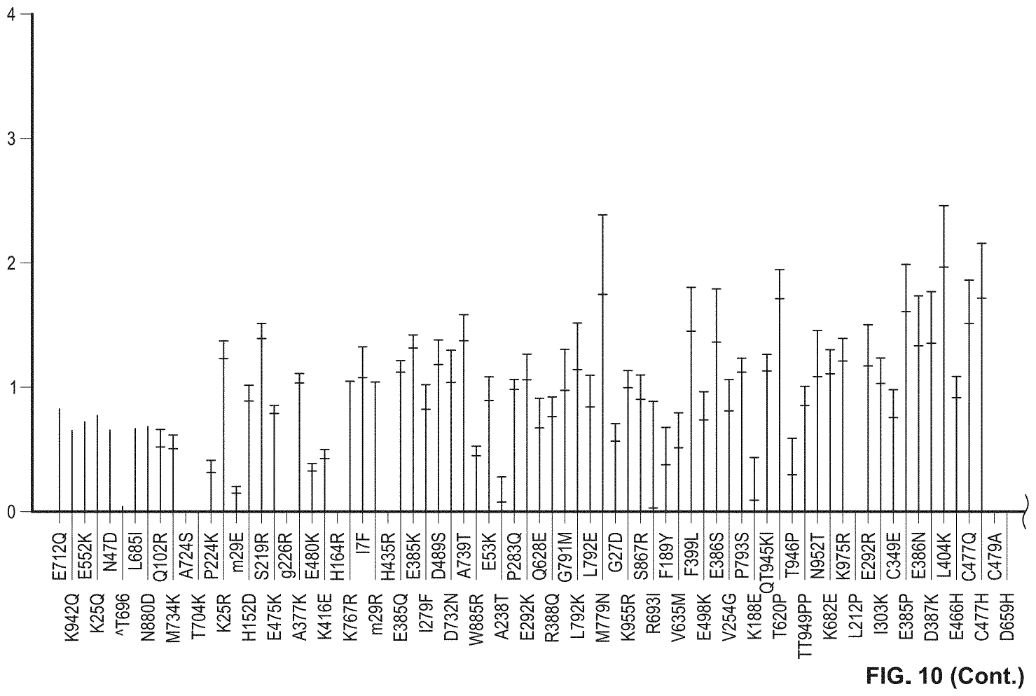

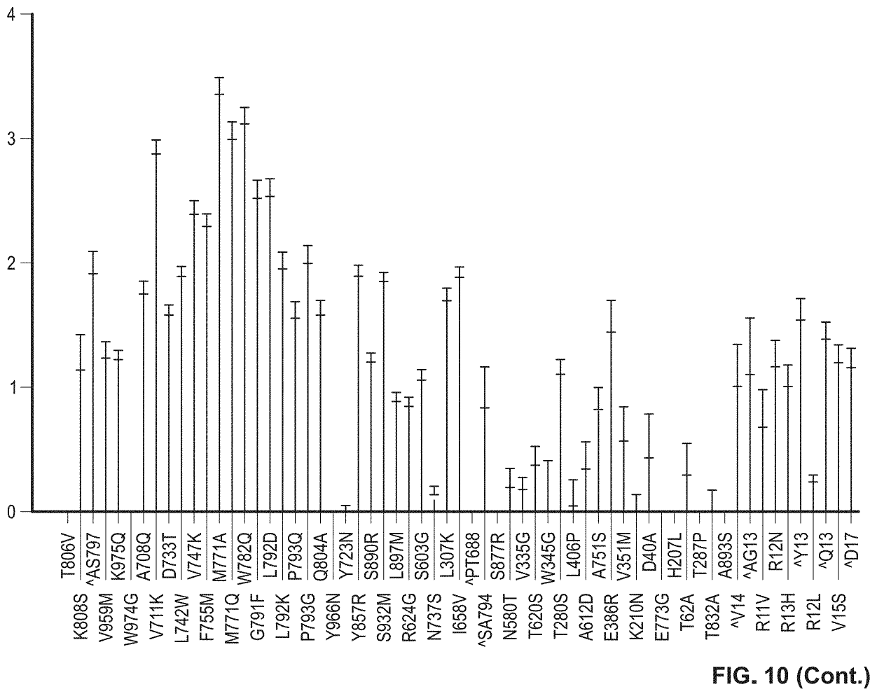

The effect of select CasX variant proteins on CasX protein activity, using a reference sgRNA scaffold (SEQ ID NO: 5) and E6 and/or E7 spacers is shown in Table 29 below and FIGS. 10 and 11.

In brief, EGFP HEK293T reporter cells were seeded into 96-well plates and transfected according to the manufacturer's protocol with lipofectamine 3000 (Life Technologies) and 50-200 ng plasmid DNA encoding the variant CasX protein, P2A-puromycin fusion and the reference sgRNA. The next day cells were selected with 1.5 μg/ml puromycin for 2 days and analyzed by fluorescence-activated cell sorting 7 days after selection to allow for clearance of EGFP protein from the cells EGFP disruption via editing was traced using an Attune NxT Flow Cytometer and high-throughput autosampler.

TABLE 29
Effect of CasX Protein Variants.

| Norm | SD | Mut. | SEQ ID NO |
|---|---|---|---|
| 3.56 | 0.479918161 | L379R + C477K + A708K + [P793] + T620P | 3866 |
| 3.44 | 0.065473567 | M771A | 3867 |
| 3.25 | 0.243066966 | L379R + A708K + [P793] + D732N | 3868 |
| 3.2 | 0.065443719 | W782Q | 3869 |
| 3.08 | 0.06581193 | M771Q | 3870 |
| 3.06 | 0.098482124 | R458I + A739V | 3871 |
| 2.99 | 0.249667198 | L379R + A708K + [P793] + M771N | 3872 |
| 2.98 | 0.226829483 | L379R + A708K + [P793] + A739T | 3873 |
| 2.98 | 0.230093698 | L379R + C477K + A708K + [P793] + D489S | 3874 |
| 2.95 | 0.225022742 | L379R + C477K + A708K + [P793] + D732N | 3875 |
| 2.95 | 0.048047426 | V711K | 3876 |
| 2.85 | 0.244869555 | L379R + C477K + A708K + [P793] + Y797L | 3877 |
| 2.84 | 0.16661152 | L379R + A708K + [P793] | 3878 |
| 2.82 | 0.219742241 | L379R + C477K + A708K + [P793] + M771N | 3879 |
| 2.75 | 0.215673641 | A708K + [P793] + E386S | 3880 |
| 2.71 | 0.10301172 | L379R + C477K + A708K + [P793] | 3881 |
| 2.62 | 0.066259269 | L792D | 3882 |
| 2.61 | 0.069056066 | G791F | 3883 |
| 2.56 | 0.138158681 | A708K + [P793] + A739V | 3884 |
| 2.52 | 0.110846334 | L379R + A708K + [P793] + A739V | 3885 |
| 2.5 | 0.070762901 | C477K + A708K + [P793] | 3886 |
| 2.47 | 0.180431811 | L249I, M771N | 3887 |
| 2.46 | 0.050035486 | V747K | 3888 |
| 2.42 | 0.14702229 | L379R + C477K + A708K + [P793] + M779N | 3889 |
| 2.36 | 0.045498608 | F755M | 3890 |
| 2.3 | 0.179759799 | L379R + A708K + [P793] + G791M | 3891 |
| 2.29 | 0.16573206 | E386R + F399L + [P793] | 3892 |
| 2.24 | 0.000278715 | A708K + [P793] | 3893 |
| 2.23 | 0.243365847 | L404K | 3894 |
| 2.16 | 0.019745961 | E552A | 3895 |
| 2.13 | 0.002238075 | A708K | 3896 |
| 2.08 | 0.316339196 | M779N | 3897 |
| 2.08 | 0.062500445 | P793G | 3898 |
| 2.07 | 0.117354932 | L379R + C477K + A708K + [P793] + A739V | 3899 |
| 2.03 | 0.057771128 | L792K | 3900 |
| 2.01 | 0.186905281 | L379R + A708K + [P793] + M779N | 3901 |
| 2.01 | 0.080358848 | \hat{ }AS797 | 3902 |
| 1.95 | 0.218366091 | C477H | 3903 |
| 1.95 | 0.040076499 | Y857R | 3904 |
| 1.94 | 0.032799694 | L742W | 3905 |
| 1.94 | 0.038256856 | I658V | 3906 |
| 1.93 | 0.055533894 | C477K + A708K + [P793] + A739V | 3907 |
| 1.9 | 0.028572575 | S932M | 3908 |
| 1.84 | 0.115143156 | T620P | 3909 |
| 1.81 | 0.18802403 | E385P | 3910 |
| 1.81 | 0.049828835 | A708Q | 3911 |
| 1.76 | 0.043121298 | L307K | 3912 |
| 1.7 | 0.03352434 | L379R + A708K + [P793] + D489S | 3913 |
| 1.7 | 0.170748704 | C477Q | 3914 |
| 1.65 | 0.051918988 | Q804A | 3915 |
| 1.64 | 0.169459451 | F399L | 3916 |
| 1.64 | 0.02984323 | L379R + A708K + [P793] + Y797L | 3917 |
| 1.64 | 0.168799771 | L379R + C477K + A708K + [P793] + G791M | 3918 |
| 1.63 | 0.035361733 | D733T | 3919 |
| 1.63 | 0.062042898 | P793Q | 3920 |
| 1.6 | 0.000928887 | A739V | 3921 |
| 1.59 | 0.208295832 | E386S | 3922 |
| 1.58 | 0.00189514 | F536S | 3923 |
| 1.57 | 0.204148363 | D387K | 3924 |
| 1.55 | 0.198137682 | E386N | 3925 |
| 1.52 | 0.000291529 | C477K | 3926 |
| 1.51 | 0.00032232 | C477R | 3927 |
| 1.49 | 0.095600844 | A739T | 3928 |
| 1.46 | 0.051799824 | S219R | 3929 |
| 1.41 | 0.000272809 | K416E & A708K | 3930 |
| 1.4 | 4.65E−05 | L379R | 3931 |
| 1.38 | 0.043395969 | E385K | 3932 |
| 1.36 | 0.000269797 | G695H | 3933 |
| 1.35 | 0.02584186 | L379R + C477K + A708K + [P793] + A739T | 3934 |
| 1.35 | 0.158192737 | E292R | 3935 |
| 1.34 | 0.184524879 | L792K | 3936 |
| 1.31 | 0.064556939 | K25R | 3937 |
| 1.31 | 0.08768015 | K975R | 3938 |
| 1.31 | 0.062237773 | V959M | 3939 |
| 1.29 | 0.092916832 | D489S | 3940 |
| 1.29 | 0.137197584 | K808S | 3941 |
| 1.28 | 0.181775511 | N952T | 3942 |
| 1.27 | 0.031730102 | K975Q | 3943 |
| 1.25 | 0.030353503 | S890R | 3944 |
| 1.23 | 0.350374014 | [P793] | 3945 |
| 1.21 | 8.61E−05 | A788W | 3946 |
| 1.21 | 0.057483618 | Q338R + A339E | 3947 |
| 1.21 | 0.116491085 | I7F | 3948 |
| 1.21 | 0.061416272 | QT945KI | 3949 |
| 1.21 | 0.091585825 | K682E | 3950 |
| 1.19 | 0.000423928 | E385A | 3951 |
| 1.19 | 0.053255444 | P793S | 3952 |
| 1.18 | 0.043774095 | E385Q | 3953 |
| 1.18 | 0.124987984 | D732N | 3954 |
| 1.17 | 0.101573595 | E292K | 3955 |
| 1.16 | 0.000245107 | S794R + Y797L | 3956 |
| 1.15 | 0.160445636 | G791M | 3957 |
| 1.14 | 0.098217225 | I303K | 3958 |
| 1.12 | 0.000275601 | \hat{ }AS793 | 3959 |
| 1.11 | 0.037923895 | S603G | 3960 |
| 1.08 | 6.48E−05 | Y797L | 3961 |
| 1.08 | 0.034990079 | A377K | 3962 |
| 1.08 | 0.059730153 | K955R | 3963 |
| 1.04 | 0.000376903 | T886K | 3964 |
| 1.03 | 0.036131932 | Q338R + A339K | 3965 |
| 1.03 | 0.031397109 | P283Q | 3966 |
| 1.01 | 0.000158685 | D600N | 3967 |
| 1.01 | 0.095937558 | S867R | 3968 |
| 1.01 | 0.079977243 | E466H | 3969 |
| 1 | 0.086320071 | E53K | 3970 |
| 0.98 | 0.123364563 | L792E | 3971 |
| 0.97 | 5.98E−05 | Q338R | 3972 |
| 0.96 | 0.059312097 | H152D | 3973 |
| 0.95 | 0.122246867 | V254G | 3974 |
| 0.94 | 0.072611815 | TT949PP | 3975 |
| 0.93 | 0.091846036 | I279F | 3976 |
| 0.93 | 0.031803852 | L897M | 3977 |
| 0.92 | 0.000288973 | K390R | 3978 |
| 0.91 | 0.000565042 | K390R | 3979 |
| 0.89 | 0.001316868 | L792G | 3980 |
| 0.89 | 0.000623156 | A739V | 3981 |
| 0.89 | 0.033874895 | R624G | 3982 |
| 0.88 | 0.103894502 | C349E | 3983 |
| 0.86 | 0.11267313 | E498K | 3984 |
| 0.85 | 0.079415017 | R388Q | 3985 |
| 0.84 | 0.000115651 | I55F | 3986 |
| 0.84 | 0.000383356 | E712Q | 3987 |
| 0.83 | 0.025220431 | E475K | 3988 |
| 0.81 | 0.000172705 | \hat{ }AS796 | 3989 |
| 0.8 | 0.111675911 | Q628E | 3990 |
| 0.79 | 0.000114918 | C479A | 3991 |
| 0.79 | 0.001115871 | Q338E | 3992 |
| 0.78 | 0.000744903 | K25Q | 3993 |
| 0.76 | 0.000269223 | \hat{ }AS795 | 3994 |
| 0.74 | 0.000437653 | L481Q | 3995 |
| 0.73 | 0.0001773 | E552K | 3996 |
| 0.72 | 0.000298273 | T153I | 3997 |
| 0.69 | 0.000273628 | N880D | 3998 |
| 0.68 | 0.000192096 | G791M | 3999 |
| 0.67 | 0.000295463 | C233S | 4000 |
| 0.67 | 0.000123996 | Q367K + I425S | 4001 |
| 0.67 | 0.000188025 | L685I | 4002 |
| 0.66 | 0.000169478 | K942Q | 4003 |
| 0.66 | 0.000374718 | N47D | 4004 |
| 0.66 | 0.138212411 | V635M | 4005 |
| 0.64 | 0.067027049 | G27D | 4006 |
| 0.63 | 0.000195863 | C479L | 4007 |
| 0.63 | 0.000439659 | [P793] + P793AS | 4008 |
| 0.62 | 0.000211625 | T72S | 4009 |
| 0.62 | 0.000217614 | S270W | 4010 |
| 0.61 | 0.00019414 | A751S | 4011 |
| 0.6 | 0.066962306 | Q102R | 4012 |
| 0.57 | 0.052391074 | M734K | 4013 |
| 0.53 | 0.000621789 | \hat{ }AS795 | 4014 |
| 0.53 | 0.145184217 | F189Y | 4015 |
| 0.5 | 0.038258832 | W885R | 4016 |
| 0.48 | 0.000505099 | A636D | 4017 |
| 0.47 | 0.030480379 | K416E | 4018 |
| 0.46 | 0.428767546 | R693I | 4019 |
| 0.45 | 0.593145404 | m29R | 4020 |
| 0.45 | 0.144374311 | T946P | 4021 |
| 0.44 | 0.000253022 | \hat{ }L889 | 4022 |
| 0.42 | 0.000171566 | E121D | 4023 |
| 0.37 | 0.042821047 | P224K | 4024 |
| 0.37 | 0.683382544 | K767R | 4025 |
| 0.36 | 0.026543344 | E480K | 4026 |
| 0.34 | 0.000998618 | I546V | 4027 |
| 0.27 | 0.164274898 | K188E | 4028 |
| 0.22 | 0.00106697 | Y789T | 4029 |
| 0.21 | 0.000512104 | F495S | 4030 |
| 0.18 | 0.023184407 | m29E | 4031 |
| 0.18 | 0.096249035 | A238T | 4032 |
| 0.17 | 0.000141352 | d231N | 4033 |
| 0.17 | 9.49E−05 | I199F | 4034 |
| 0.17 | 0.031218317 | N737S | 4035 |
| 0.16 | 3.87E−05 | \hat{ }G661A | 4036 |
| 0.12 | 4.08E−05 | K460N | 4037 |
| 0.08 | 0.000897639 | k210R | 4038 |
| 0.08 | 3.47E−05 | G492P | 4039 |
| 0.07 | 0.000266253 | R591I | 4040 |
| 0.04 | 6.41E−05 | \hat{ }T696 | 4041 |
| 0.03 | 0.022802297 | S507G + G508R | 4042 |
| 0.02 | 0.028138538 | Y723N | 4043 |
| −0.01 | 0.000529731 | \hat{ }P696 | 4044 |
| −0.01 | 0.038340599 | g226R | 4045 |
| −0.02 | 0.052026759 | W974G | 4046 |
| −0.04 | 0.000176981 | \hat{ }M773 | 4047 |
| −0.04 | 0.07902452 | H435R | 4048 |
| −0.06 | 0.069143378 | A724S | 4049 |
| −0.06 | 0.060317972 | T704K | 4050 |
| −0.06 | 0.017155351 | Y966N | 4051 |
| −0.08 | 0.036299549 | H164R | 4052 |
| −0.15 | 0.032952207 | F556I, D646A, G695D, A751S, A820P | 4053 |
| −0.17 | 0.04149111 | D659H | 4054 |
| −0.21 | 0.064777446 | T806V | 4055 |
| −0.24 | 0.001280151 | Y789D | 4056 |
| −0.31 | 0.05332531 | C479A | 4057 |
| −0.35 | 0.066448437 | L212P | 4058 |

Norm = Normalized Editing Activity (avg, 2 spacer n = 6); SD = Standard Deviation; Mut = Mutation Descriptor.
Mutations are relative to SEQ ID NO: 2.
[ ] indicate deletions, and (\hat{ }) indicate insertions at the specified positions of SEQ ID NO: 2.
E6 and E7 spacers were used, and the data are the average of N = 6 replicates.
St. Dev. = Standard Deviation.
Editing activity was normalized to that of the reference CasX protein of SEQ ID NO: 2.

Selected CasX variant proteins from the DME screen and CasX variant proteins comprising combinations of mutations were assayed for their ability to disrupt via cleavage and indel formation GFP reporter expression. CasX variant proteins were assayed with two targets, with 6 replicates. FIG. 10 shows the fold improvement in activity over the reference CasX protein of SEQ ID NO: 2 of select variants carrying single mutations, assayed with the reference sgRNA scaffold of SEQ ID NO: 5.

FIG. 11 shows that combining single mutations, such as those shown in FIG. 10, can produce CasX variant proteins, that can improve editing efficiency by greater than two-fold. The most improved CasX variant proteins, which combine 3 or 4 individual mutations, exhibit activity comparable to Staphylococcus aureus Cas9 (SaCas9) which is used in the clinic (Maeder et al. 2019, Nature Medicine 25(2):229-233).

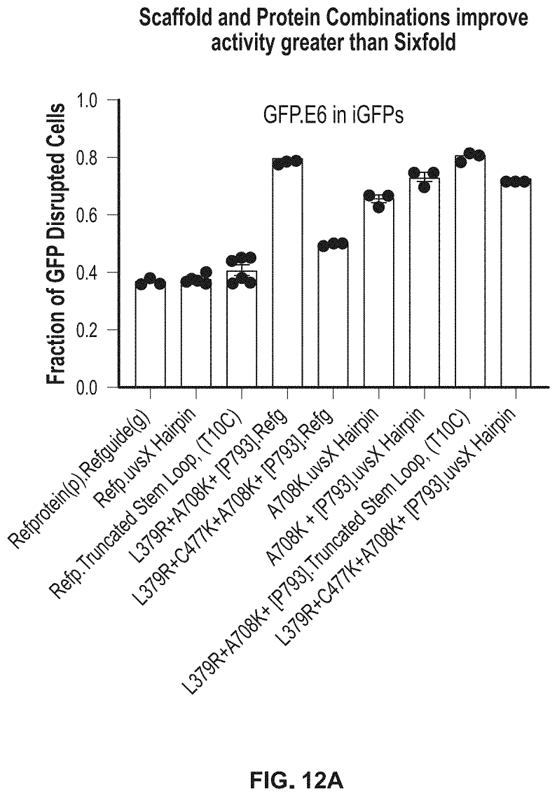

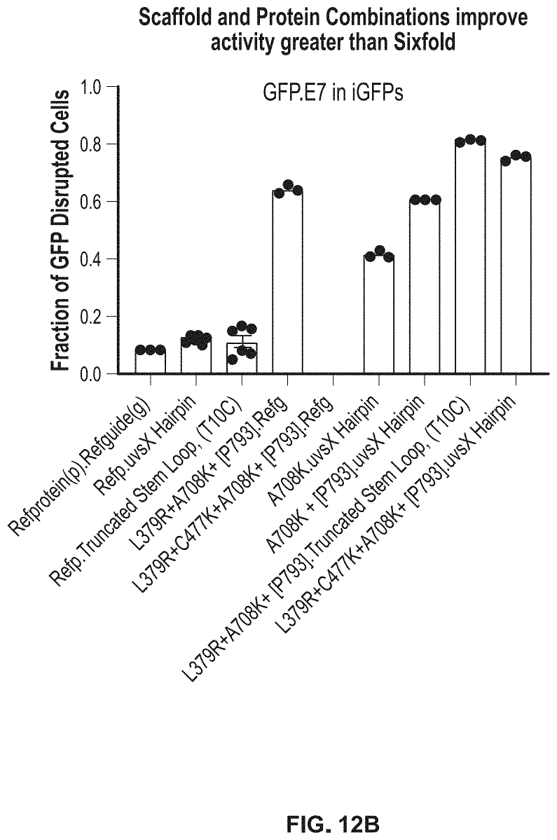

FIGS. 12A-12B shows that CasX variant proteins, when combined with select sgRNA variants, can achieve even greater improvements in editing efficiency. For example, a protein variant comprising L379K and A708K substitutions, and a P793 deletion of SEQ ID NO: 2, when combined with the truncated stem loop T10C sgRNA variant more than doubles the fraction of disrupted cells.

### Example 6: RNP Assembly

Purified wild-type and RNP of CasX and single guide RNA (sgRNA) were either prepared immediately before experiments or prepared and snap-frozen in liquid nitrogen and stored at −80° C. for later use. To prepare the RNP complexes, the CasX protein was incubated with sgRNA at 1:1.2 molar ratio. Briefly, sgRNA was added to Buffer #1 (25 mM NaPi, 150 mM NaCl, 200 mM trehalose, 1 mM MgCl2), then the CasX was added to the sgRNA solution, slowly with swirling, and incubated at 37° C. for 10 min to form RNP complexes. RNP complexes were filtered before use through a 0.22 μm Costar 8160 filters that were pre-wet with 200111 Buffer #1. If needed, the RNP sample was concentrated with a 0.5 ml Ultra 100-Kd cutoff filter, (Millipore part #UFC510096), until the desired volume was obtained. Formation of competent RNP was assessed as described in Example 12.

### Example 7: Assessing Binding Affinity to the Guide RNA

Purified wild-type and improved CasX will be incubated with synthetic single-guide RNA containing a 3′ Cy7.5 moiety in low-salt buffer containing magnesium chloride as well as heparin to prevent non-specific binding and aggregation. The sgRNA will be maintained at a concentration of 10 pM, while the protein will be titrated from 1 pM to 100 μM in separate binding reactions. After allowing the reaction to come to equilibrium, the samples will be run through a vacuum manifold filter-binding assay with a nitrocellulose membrane and a positively charged nylon membrane, which bind protein and nucleic acid, respectively. The membranes will be imaged to identify guide RNA, and the fraction of bound vs unbound RNA will be determined by the amount of fluorescence on the nitrocellulose vs nylon membrane for each protein concentration to calculate the dissociation constant of the protein-sgRNA complex. The experiment will also be carried out with improved variants of the sgRNA to determine if these mutations also affect the affinity of the guide for the wild-type and mutant proteins. We will also perform electromobility shift assays to qualitatively compare to the filter-binding assay and confirm that soluble binding, rather than aggregation, is the primary contributor to protein-RNA association.

### Example 8: Assessing Binding Affinity to the Target DNA

Purified wild-type and improved CasX will be complexed with single-guide RNA bearing a targeting sequence complementary to the target nucleic acid. The RNP complex will be incubated with double-stranded target DNA containing a PAM and the appropriate target nucleic acid sequence with a 5′ Cy7.5 label on the target strand in low-salt buffer containing magnesium chloride as well as heparin to prevent non-specific binding and aggregation. The target DNA will be maintained at a concentration of 1 nM, while the RNP will be titrated from 1 pM to 100 μM in separate binding reactions. After allowing the reaction to come to equilibrium, the samples will be run on a native 5% polyacrylamide gel to separate bound and unbound target DNA. The gel will be imaged to identify mobility shifts of the target DNA, and the fraction of bound vs unbound DNA will be calculated for each protein concentration to determine the dissociation constant of the RNP-target DNA ternary complex.

### Example 9: Assessing Differential PAM Recognition In Vitro

Purified wild-type and engineered CasX variants will be complexed with single-guide RNA bearing a fixed targeting sequence. The RNP complexes will be added to buffer containing MgCl2 at a final concentration of 100 nM and incubated with 5′ Cy7.5-labeled double-stranded target DNA at a concentration of 10 nM. Separate reactions will be carried out with different DNA substrates containing different PAMs adjacent to the target nucleic acid sequence. Aliquots of the reactions will be taken at fixed time points and quenched by the addition of an equal volume of 50 mM EDTA and 95% formamide. The samples will be run on a denaturing polyacrylamide gel to separate cleaved and uncleaved DNA substrates. The results will be visualized and the rate of cleavage of the non-canonical PAMs by the CasX variants will be determined.

### Example 10: Assessing Nuclease Activity for Double-Strand Cleavage

Purified wild-type and engineered CasX variants will be complexed with single-guide RNA bearing a fixed PM22 targeting sequence. The RNP complexes will be added to buffer containing MgCl2 at a final concentration of 100 nM and incubated with double-stranded target DNA with a 5′ Cy7.5 label on either the target or non-target strand at a concentration of 10 nM. Aliquots of the reactions will be taken at fixed time points and quenched by the addition of an equal volume of 50 mM EDTA and 95% formamide. The samples will be run on a denaturing polyacrylamide gel to separate cleaved and uncleaved DNA substrates. The results will be visualized and the cleavage rates of the target and non-target strands by the wild-type and engineered variants will be determined. To more clearly differentiate between changes to target binding vs the rate of catalysis of the nucleolytic reaction itself, the protein concentration will be titrated over a range from 10 nM to 1 uM and cleavage rates will be determined at each concentration to generate a pseudo-Michaelis-Menten fit and determine the kcat* and KM*. Changes to KM* are indicative of altered binding, while changes to kcat* are indicative of altered catalysis.

### Example 11: Assessing Target Strand Loading for Cleavage

Purified wild-type and engineered CasX 119 will be complexed with single-guide RNA bearing a fixed PM22 targeting sequence. The RNP complexes will be added to buffer containing MgCl2 at a final concentration of 100 nM and incubated with double-stranded target DNA with a 5′ Cy7.5 label on the target strand and a 5′ Cy5 label on the non-target strand at a concentration of 10 nM. Aliquots of the reactions will be taken at fixed time points and quenched by the addition of an equal volume of 50 mM EDTA and 95% formamide. The samples will be run on a denaturing polyacrylamide gel to separate cleaved and uncleaved DNA substrates. The results will be visualized and the cleavage rates of both strands by the variants will be determined. Changes to the rate of target strand cleavage but not non-target strand cleavage would be indicative of improvements to the loading of the target strand in the active site for cleavage. This activity could be further isolated by repeating the assay with a dsDNA substrate that has a gap on the non-target strand, mimicking a pre-cleaved substrate. Improved cleavage of the non-target strand in this context would give further evidence that the loading and cleavage of the target strand, rather than an upstream step, has been improved.

### Example 12: CasX:gNA In Vitro Cleavage Assays

1. Determining Cleavage-competent Fraction

The ability of CasX variants to form active RNP compared to reference CasX was determined using an in vitro cleavage assay. The beta-2 microglobulin (B2M) 7.37 target for the cleavage assay was created as follows. DNA oligos with the sequence TGAAGCTGACAGCATTCGGGCCGAGATGTCTCGCTCCGTGGCCTTAGCTGTGCTCGC GCT (SEQ ID NO: 4059; non-target strand, NTS) and TGAAGCTGACAGCATTCGGGCCGAGATGTCTCGCTCCGTGGCCTTAGCTGTGCTCGC GCT (SEQ ID NO: 4060; target strand, TS) were purchased with 5′ fluorescent labels (LI-COR IRDye 700 and 800, respectively). dsDNA targets were formed by mixing the oligos in a 1:1 ratio in 1× cleavage buffer (20 mM Tris HCl pH 7.5, 150 mM NaCl, 1 mM TCEP, 5% glycerol, 10 mM MgCl₂), heating to 95° C. for 10 minutes, and allowing the solution to cool to room temperature.

CasX RNPs were reconstituted with the indicated CasX and guides (see graphs) at a final concentration of 1 μM with 1.5-fold excess of the indicated guide in 1× cleavage buffer (20 mM Tris HCl pH 7.5, 150 mM NaCl, 1 mM TCEP, 5% glycerol, 10 mM MgCl2) at 37° C. for 10 min before being moved to ice until ready to use. The 7.37 target was used, along with sgRNAs having spacers complementary to the 7.37 target.

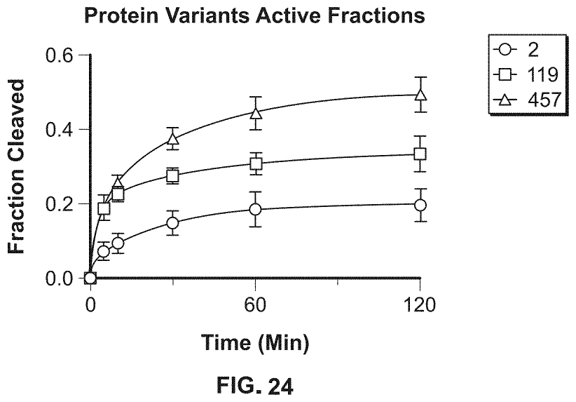

Cleavage reactions were prepared with final RNP concentrations of 100 nM and a final target concentration of 100 nM. Reactions were carried out at 37° C. and initiated by the addition of the 7.37 target DNA. Aliquots were taken at 5, 10, 30, 60, and 120 minutes and quenched by adding to 95% formamide, 20 mM EDTA. Samples were denatured by heating at 95° C. for 10 minutes and run on a 10% urea-PAGE gel. The gels were imaged with a LI-COR Odyssey CLx and quantified using the LI-COR Image Studio software. The resulting data were plotted and analyzed using Prism. We assumed that CasX acts as essentially as a single-turnover enzyme under the assayed conditions, as indicated by the observation that sub-stoichiometric amounts of enzyme fail to cleave a greater-than-stoichiometric amount of target even under extended time-scales and instead approach a plateau that scales with the amount of enzyme present. Thus, the fraction of target cleaved over long time-scales by an equimolar amount of RNP is indicative of what fraction of the RNP is properly formed and active for cleavage. The cleavage traces were fit with a biphasic rate model, as the cleavage reaction clearly deviates from monophasic under this concentration regime, and the plateau was determined for each of three independent replicates. The mean and standard deviation were calculated to determine the active fraction (Table 30). The graphs are shown in FIG. 24.

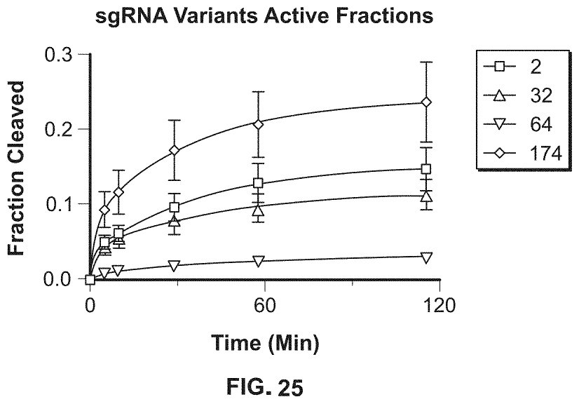

Apparent active (competent) fractions were determined for RNPs formed for CasX2+guide 174+7.37 spacer, CasX119+guide 174+7.37 spacer, and CasX459+guide 174+7.37 spacer. The determined active fractions are shown in Table 30. Both CasX variants had higher active fractions than the wild-type CasX2, indicating that the engineered CasX variants form significantly more active and stable RNP with the identical guide under tested conditions compared to wild-type CasX. This may be due to an increased affinity for the sgRNA, increased stability or solubility in the presence of sgRNA, or greater stability of a cleavage-competent conformation of the engineered CasX:sgRNA complex. An increase in solubility of the RNP was indicated by a notable decrease in the observed precipitate formed when CasX457 was added to the sgRNA compared to CasX2. Cleavage-competent fractions were also determined for CasX2.2.7.37, CasX2.32.7.37, CasX2.64.7.37, and CasX2.174.7.37 to be 16±3%, 13±3%, 5±2%, and 22±5%, as shown in FIG. 25.

The data indicate that both CasX variants and sgRNA variants are able to form a higher degree of active RNP with guide RNA compare to wild-type CasX and wild-type sgRNA. 2. In vitro Cleavage Assays—Determining kcleave for CasX variants compared to wild-type reference CasX

The apparent cleavage rates of CasX variants 119 and 457 compared to wild-type reference CasX were determined using an in vitro fluorescent assay for cleavage of the target 7.37.

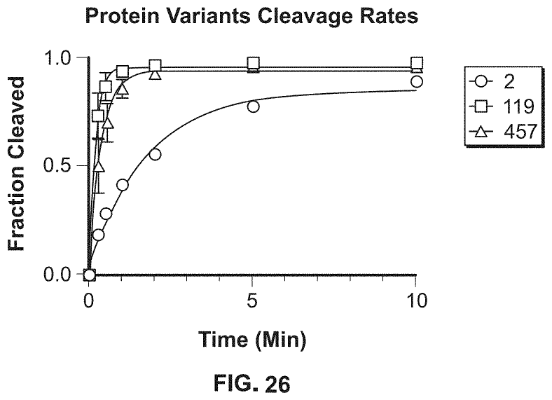

CasX RNPs were reconstituted with the indicated CasX (see FIG. 26) at a final concentration of 1 μM with 1.5-fold excess of the indicated guide in 1× cleavage buffer (20 mM Tris HCl pH 7.5, 150 mM NaCl, 1 mM TCEP, 5% glycerol, 10 mM MgCl2) at 37° C. for 10 min before being moved to ice until ready to use. Cleavage reactions were set up with a final RNP concentration of 200 nM and a final target concentration of 10 nM. Reactions were carried out at 37° C. and initiated by the addition of the target DNA. Aliquots were taken at 0.25, 0.5, 1, 2, 5, and 10 minutes and quenched by adding to 95% formamide, 20 mM EDTA. Samples were denatured by heating at 95° C. for 10 minutes and run on a 10% urea-PAGE gel. The gels were imaged with a LI-COR Odyssey CLx and quantified using the LI-COR Image Studio software. The resulting data were plotted and analyzed using Prism, and the apparent first-order rate constant of non-target strand cleavage (kcleave) was determined for each CasX:sgRNA combination replicate individually. The mean and standard deviation of three replicates with independent fits are presented in Table 30, and the cleavage traces are shown in FIG. 25.

Apparent cleavage rate constants were determined for wild-type CasX2, and CasX variants 119 and 457 with guide 174 and spacer 7.37 utilized in each assay. Under the assayed conditions, the kcleave of CasX2, CasX119, and CasX457 were 0.51±0.01 min-1, 6.29±2.11 min-1, and 3.01±0.90 min-1 (mean±SD), respectively (see Table 30 and FIG. 26). Both CasX variants had improved cleavage rates relative to the wild-type CasX2, though notably CasX119 has a higher cleavage rate under tested conditions than CasX457. As demonstrated by the active fraction determination, however, CasX457 more efficiently forms stable and active RNP complexes, allowing different variants to be used depending on whether the rate of cutting or the amount of active holoenzyme is more important for the desired outcome.

The data indicate that the CasX variants have a higher level of activity, with Kcleave rates approximately 5 to 10-fold higher compared to wild-type CasX2. 3. In vitro Cleavage Assays: Comparison of guide variants to wild-type guides

Cleavage assays were also performed with wild-type reference CasX2 and reference guide 2 compared to guide variants 32, 64, and 174 to determine whether the variants improved cleavage. The experiments were performed as described above. As many of the resulting RNPs did not approach full cleavage of the target in the time tested, we determined initial reaction velocities (VO) rather than first-order rate constants. The first two timepoints (15 and 30 seconds) were fit with a line for each CasX:sgRNA combination and replicate. The mean and standard deviation of the slope for three replicates were determined.

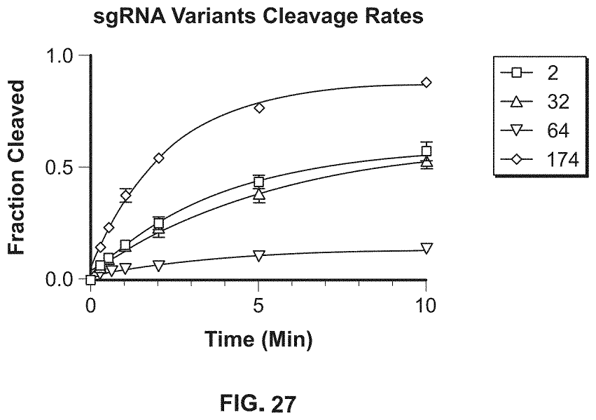

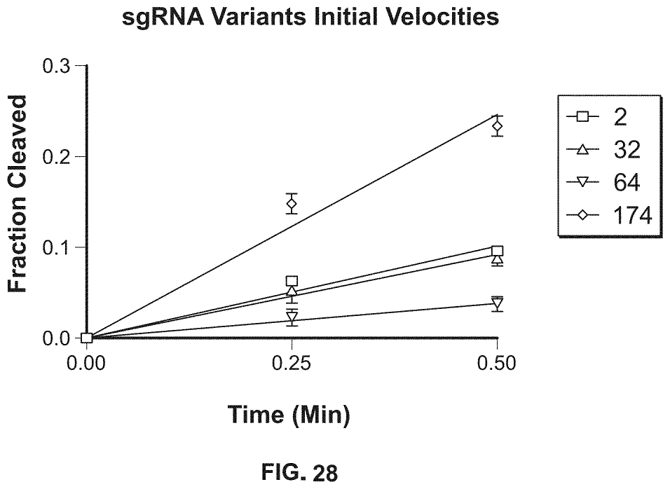

Under the assayed conditions, the VO for CasX2 with guides 2, 32, 64, and 174 were 20.4±1.4 nM/min, 18.4±2.4 nM/min, 7.8±1.8 nM/min, and 49.3±1.4 nM/min (see Table 30 and FIG. 27). Guide 174 showed substantial improvement in the cleavage rate of the resulting RNP (˜2.5-fold relative to 2, see FIG. 28), while guides 32 and 64 performed similar to or worse than guide 2. Notably, guide 64 supports a cleavage rate lower than that of guide 2 but performs much better in vivo (data not shown). Some of the sequence alterations to generate guide 64 likely improve in vivo transcription at the cost of a nucleotide involved in triplex formation. Improved expression of guide 64 likely explains its improved activity in vivo, while its reduced stability may lead to improper folding in vitro.

TABLE 30
Results of cleavage and RNP formation assays

| RNP | | Initial | Competent |
|---|---|---|---|
| Construct | k_{cleave}* | velocity* | fraction |
| 2.2.7.37 | | 20.4 ± 1.4 nM/min | 16 ± 3% |
| 2.32.7.37 | | 18.4 ± 2.4 nM/min | 13 ± 3% |
| 2.64.7.37 | | 7.8 ± 1.8 nM/min | 5 ± 2% |
| 2.174.7.37 | 0.51 ± 0.01 min⁻¹ | 49.3 ± 1.4 nM/min | 22 ± 5% |
| 119.174.7.37 | 6.29 ± 2.11 min⁻¹ | | 35 ± 6% |
| 457.174.7.37 | 3.01 ± 0.90 min⁻¹ | | 53 ± 7% |

*Mean and standard deviation

### Example 13: CasX Variant Proteins can Affect PAM Specificity

The purpose of the experiment was to demonstrate the ability of CasX variant 2 (SEQ ID NO:2), and scaffold variant 2 (SEQ ID NO:5), to edit target gene sequences at ATCN, CTCN, and TTCN PAMs in a GFP gene. ATCN, CTCN, and TTCN spacers in the GFP gene were chosen based on PAM availability without prior knowledge of potential activity.

To facilitate assessment of editing outcomes, HEK293T-GFP reporter cell line was first generated by knocking into HEK293T cells a transgene cassette that constitutively. expresses GFP. The modified cells were expanded by serial passage every 3-5 days and maintained in Fibroblast (FB) medium, consisting of Dulbecco's Modified Eagle Medium (DMEM; Corning Cellgro, #10-013-CV) supplemented with 10% fetal bovine serum (FBS; Seradigm, #1500-500), and 100 Units/mL penicillin and 100 mg/mL streptomycin (100×-Pen-Strep; GIBCO #15140-122), and can additionally include sodium pyruvate (100×, Thermofisher #11360070), non-essential amino acids (100× Thermofisher #11140050), HEPES buffer (100× Thermofisher #15630080), and 2-mercaptoethanol (1000× Thermofisher #21985023). The cells were incubated at 37° C. and 5% CO2. After 1-2 weeks, GFP+ cells were bulk sorted into FB medium. The reporter lines were expanded by serial passage every 3-5 days and maintained in FB medium in an incubator at 37° C. and 5% CO2. Clonal cell lines were generated by a limiting dilution method.

HEK293T-GFP reporter cells, constructed using cell line generation methods described above were used for this experiment. Cells were seeded at 20-40k cells/well in a 96 well plate in 100 μL of FB medium and cultured in a 37*C incubator with 5% CO2. The following day, cells were transfected at ˜75% confluence using lipofectamine 3000 and manufacturer recommended protocols. Plasmid DNA encoding CasX and guide construct (e.g., see table for sequences) were used to transfect cells at 100-400 ng/well, using 3 wells per construct as replicates. A non-targeting plasmid construct was used as a negative control. Cells were selected for successful transfection with puromycin at 0.3-3 μg/ml for 24-48 hours followed by recovery in FB medium. Edited cells were analyzed by flow cytometry 5 days after transduction. Briefly, cells were sequentially gated for live cells, single cells, and fraction of GFP-negative cells.

### Results:

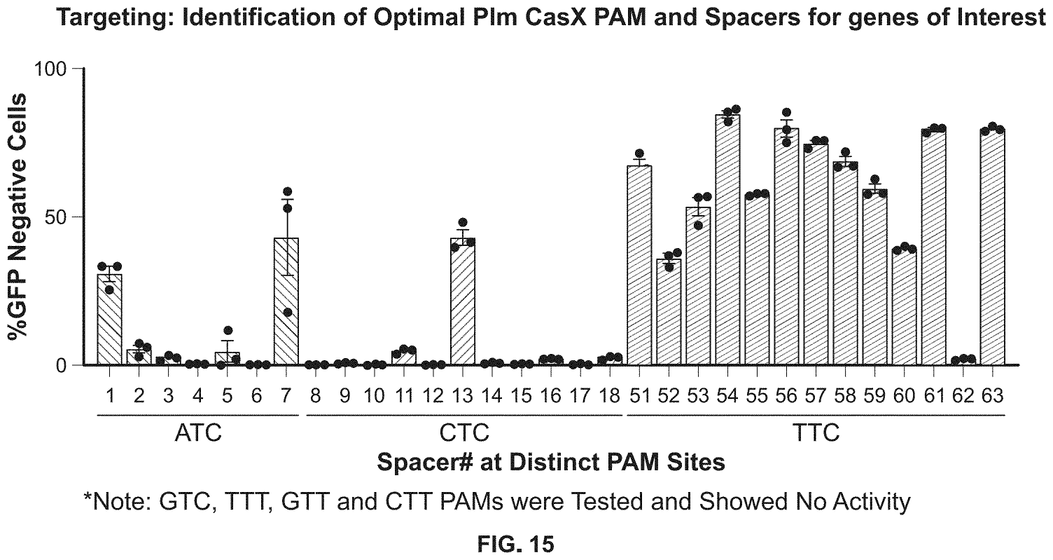

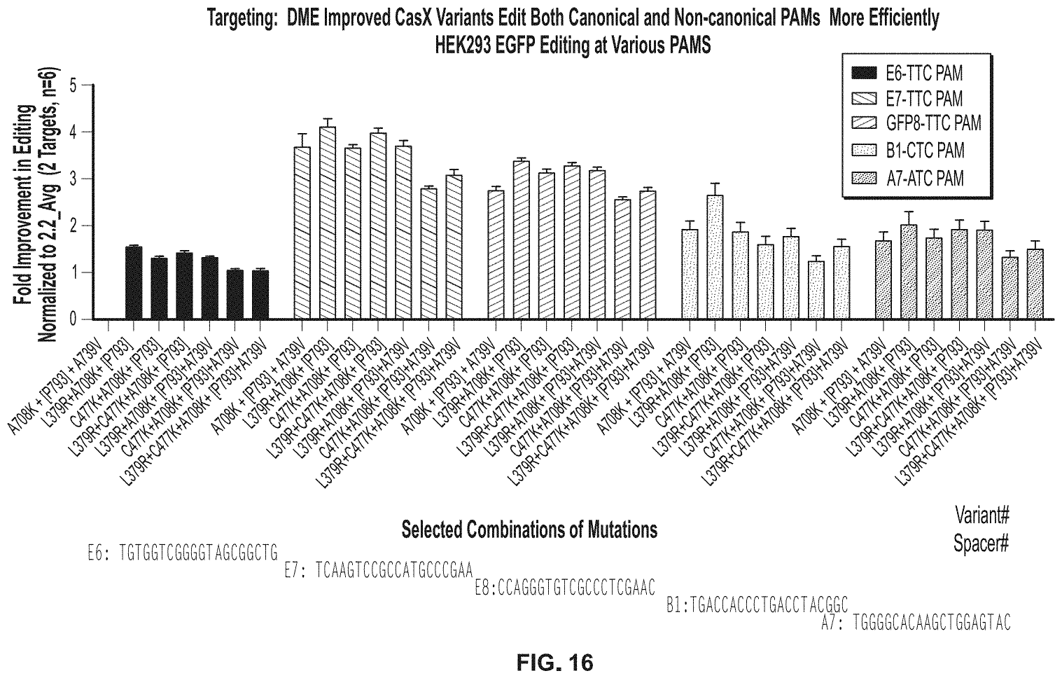

The graph in FIG. 15 shows the results of flow cytometry analysis of Cas-mediated editing at the GFP locus in HEK293T-GFP cells 5 days post-transfection. Each data point is an average measurement of 3 replicates for an individual spacer. Reference CasX reference protein (SEQ ID NO: 2) and gRNA (SEQ ID NO: 5) RNP complexes showed a clear preference for TTC PAM (FIG. 15). This served as a baseline for CasX protein and sgRNA variants that altered specificity for the PAM sequence. FIG. 16 shows that select CasX variant proteins can edit both non-canonical and canonical PAM sequences more efficiently than the reference CasX protein of SEQ ID NO: 2 when assayed with various PAM and spacer sequences in HEK293 cells. The construct with non-targeting spacer resulted in no editing (data not shown). This example demonstrates that, under the conditions of the assay, CasX with appropriate guides can edit at target sequences with ATCN, CTCN and TTCN PAMs in HEK293T-GFP reporter cells, and that improved CasX variants increase editing activity at both canonical and non-canonical PAMs.

### Example 14: Reference Planctomycetes CasX RNPs are Highly Specific

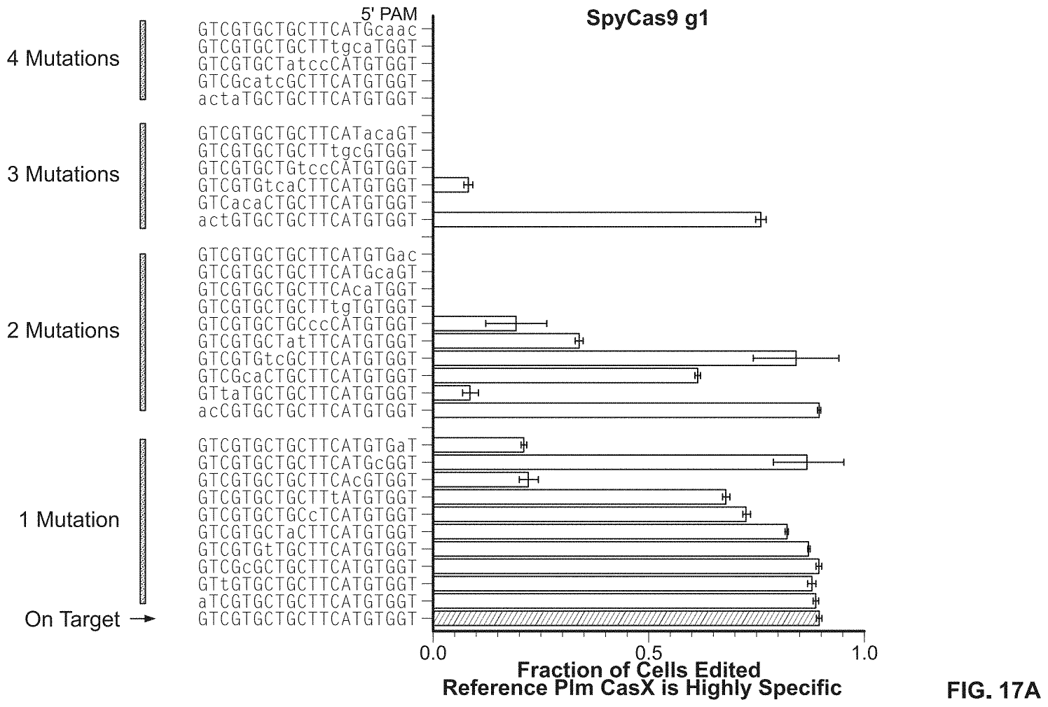

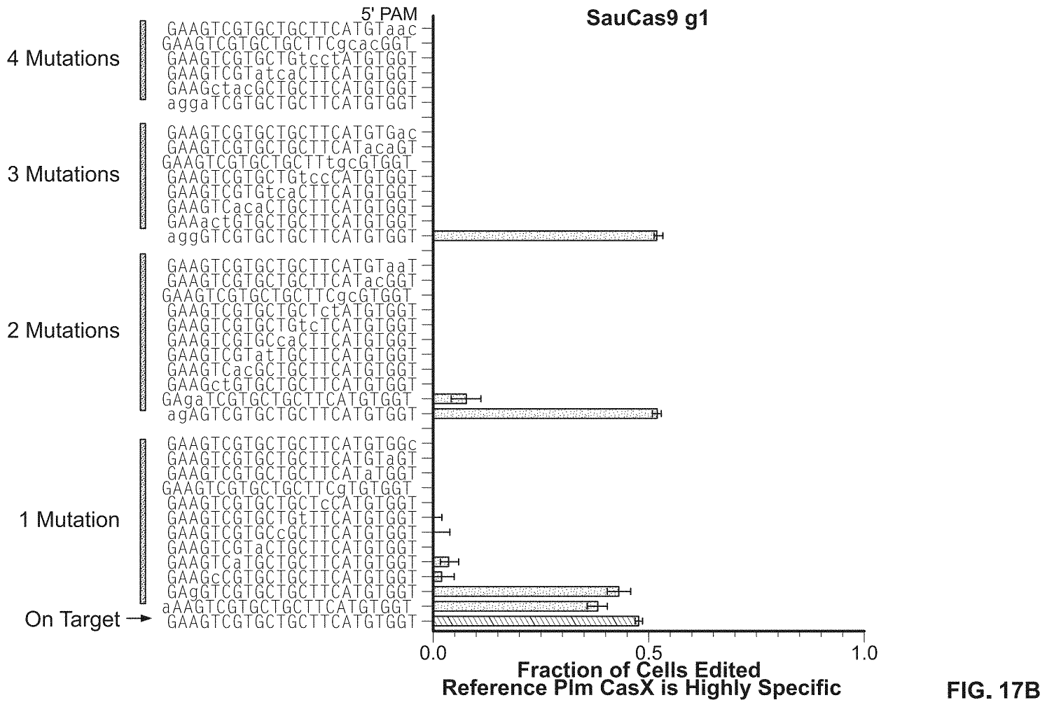

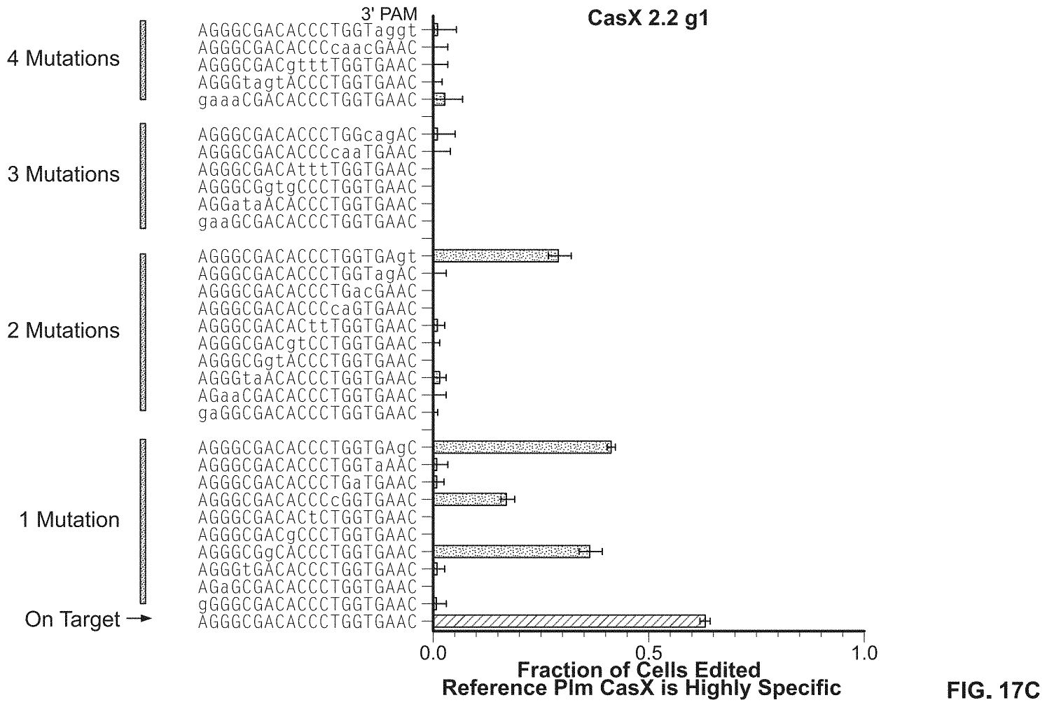

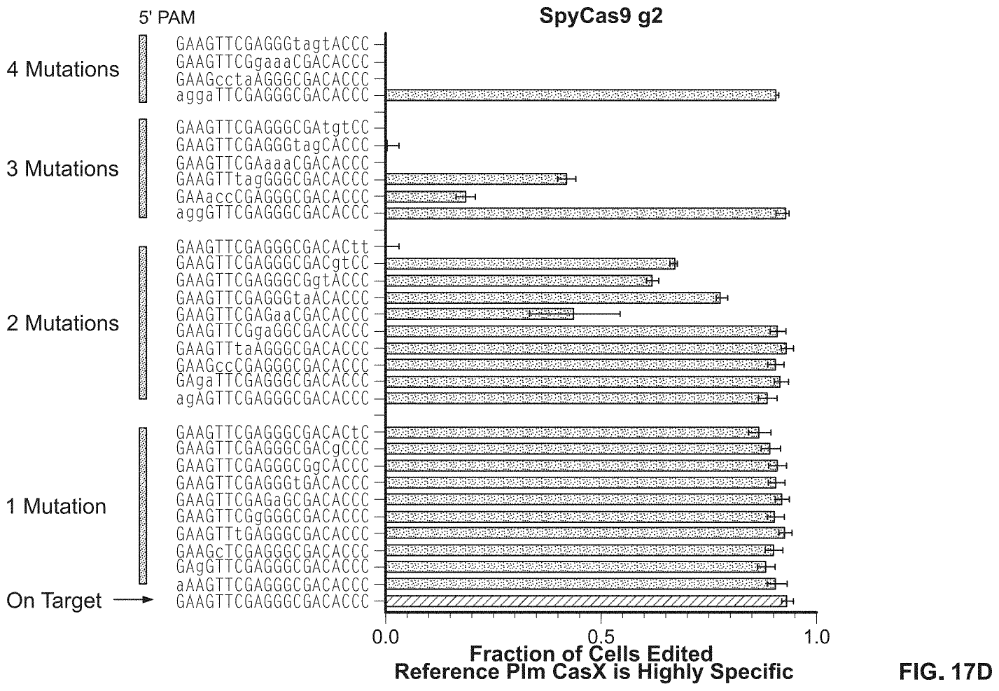

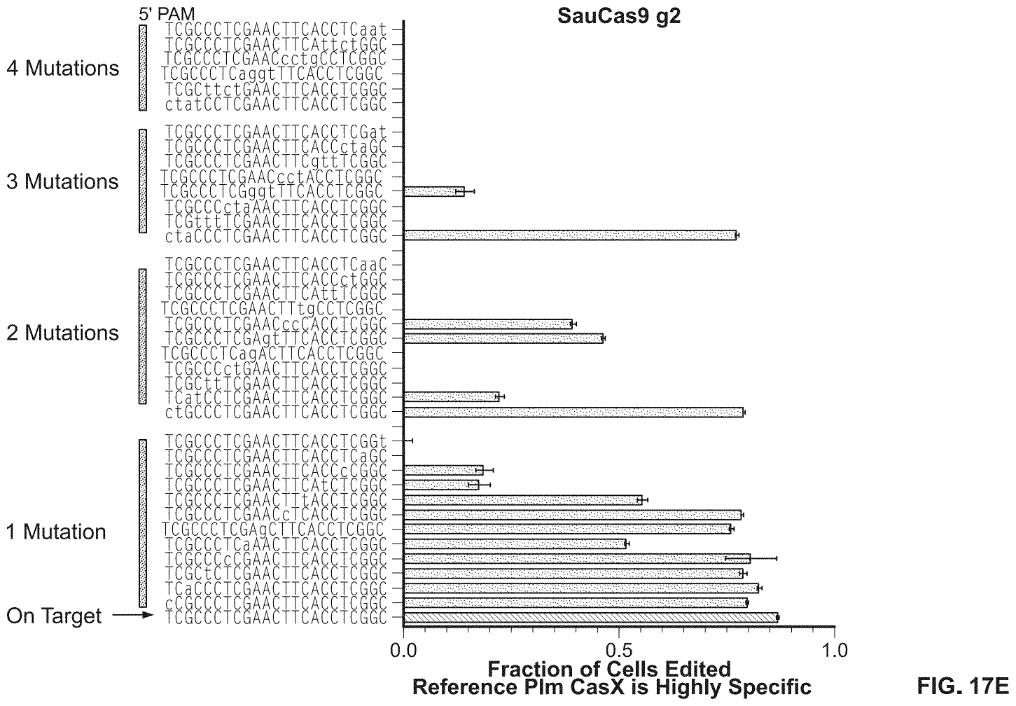

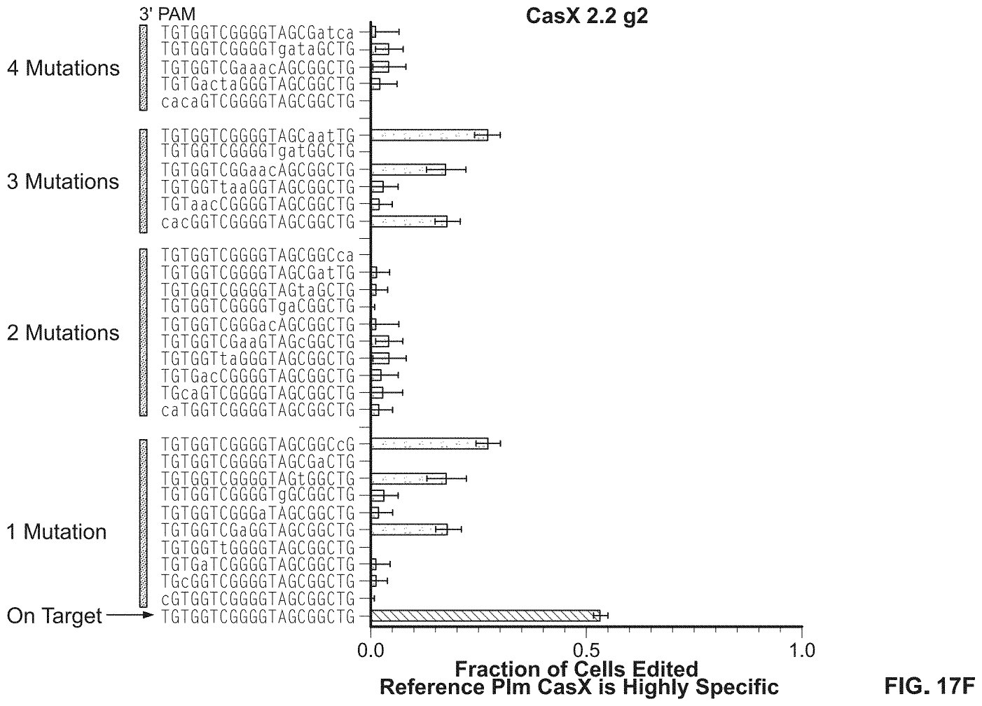

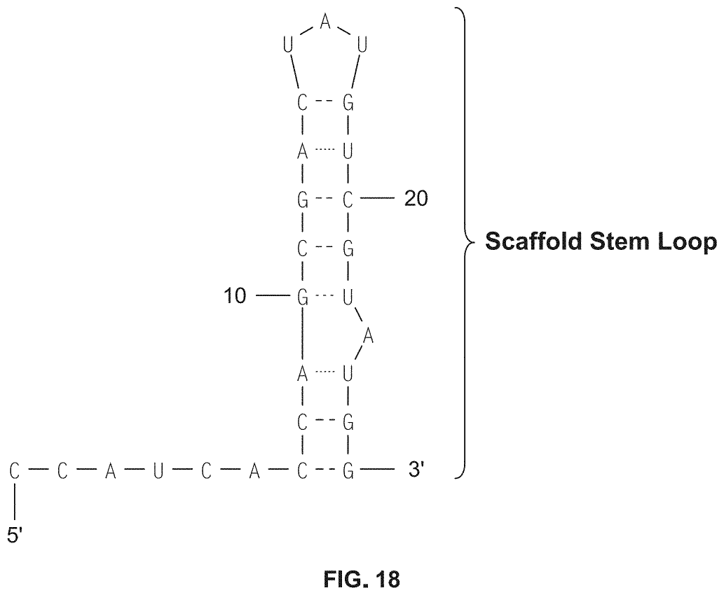

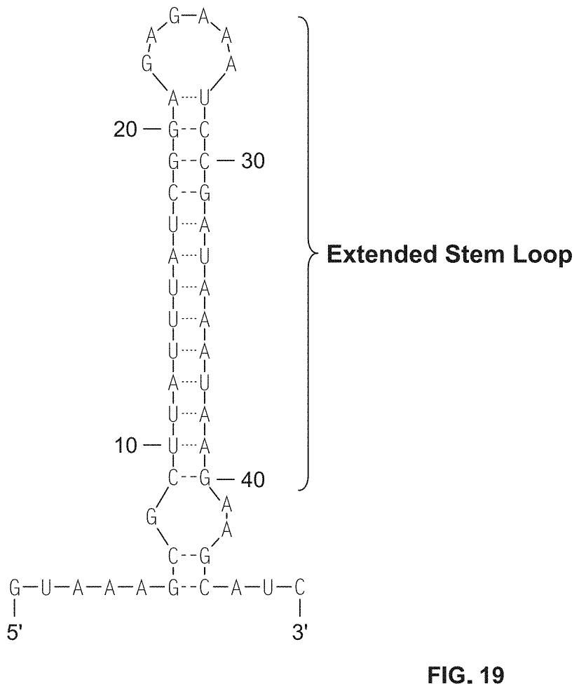

Reference CasX RNP complexes were assayed for their ability to cleave target sequences with 1-4 mutations, with results shown in FIGS. 17A-17F. Reference Planctomycetes CasX RNPs were found to be highly specific and exhibited fewer off-target effects than SpCas9 and SauCas9.

### Example 15: Editing of gene targets PCSK9, PMP22, TRAC, SOD1, B2M and HTT

The purpose of this study was to evaluate the ability of the CasX variant 119 and gNA variant 174 to edit nucleic acid sequences in six gene targets.

### Materials and Methods

Spacers for all targets except B2M and SOD1 were designed in an unbiased manner based on PAM requirements (TTC or CTC) to target a desired locus of interest. Spacers targeting B2M and SOD1 had been previously identified within targeted exons via lentiviral spacer screens carried out for these genes. Designed spacers for the other targets were ordered from Integrated DNA Technologies (IDT) as single-stranded DNA (ssDNA) oligo pairs. ssDNA spacer pairs were annealed together and cloned via Golden Gate cloning into a base mammalian-expression plasmid construct that contains the following components: codon optimized Cas X 119 protein+NLS under an EF1A promoter, guide scaffold 174 under a U6 promoter, carbenicillin and puromycin resistance genes. Assembled products were transformed into chemically-competent E. coli, plated on Lb-Agar plates (LB: Teknova Cat #L9315, Agar: Quartzy Cat #214510) containing carbenicillin and incubated at 37° C. Individual colonies were picked and miniprepped using Qiagen Qiaprep spin Miniprep Kit (Qiagen Cat #27104) following the manufacturer's protocol. The resulting plasmids were sequenced through the guide scaffold region via Sanger sequencing (Quintara Biosciences) to ensure correct ligation.

HEK 293T cells were grown in Dulbecco's Modified Eagle Medium (DMEM; Corning Cellgro, #10-013-CV) supplemented with 10% fetal bovine serum (FBS; Seradigm, #1500-500), 100 Units/ml penicillin and 100 mg/ml streptomycin (100×-Pen-Strep; GIBCO #15140-122), sodium pyruvate (100×, Thermofisher #11360070), non-essential amino acids (100× Thermofisher #11140050), HEPES buffer (100× Thermofisher #15630080), and 2-mercaptoethanol (1000× Thermofisher #21985023). Cells were passed every 3-5 days using Tryp1E and maintained in an incubator at 37° C. and 5% CO2.

On day 0, HEK293T cells were seeded in 96-well, flat-bottom plates at 30k cells/well. On day 1, cells were transfected with 100 ng plasmid DNA using Lipofectamine 3000 according to the manufacturer's protocol. On day 2, cells were switched to FB medium containing puromycin. On day 3, this media was replaced with fresh FB medium containing puromycin. The protocol after this point diverged depending on the gene of interest. Day 4 for PCSK9, PMP22, and TRAC: cells were verified to have completed selection and switched to FB medium without puromycin. Day 4 for B2M, SOD1, and HTT: cells were verified to have completed selection and passed 1:3 using Tryp1E into new plates containing FB medium without puromycin. Day 7 for PCSK9, PMP22, and TRAC: cells were lifted from the plate, washed in dPBS, counted, and resuspended in Quick Extract (Lucigen, QE09050) at 10,000 cells/μ1. Genomic DNA was extracted according to the manufacturer's protocol and stored at −20° C. Day 7 for B2M, SOD1, and HTT: cells were lifted from the plate, washed in dPBS, and genomic DNA was extracted with the Quick-DNA Miniprep Plus Kit (Zymo, D4068) according to the manufacturer's protocol and stored at −20° C.

NGS Analysis: Editing in cells from each experimental sample was assayed using next generation sequencing (NGS) analysis. All PCRs were carried out using the KAPA HiFi HotStart ReadyMix PCR Kit (KR0370). The template for genomic DNA sample PCR was 5 μl of genomic DNA in QE at 10k cells/μL for PCSK9, PMP22, and TRAC. The template for genomic DNA sample PCR was 400 ng of genomic DNA in water for B2M, SOD1, and HTT. Primers were designed specific to the target genomic location of interest to form a target amplicon. These primers contain additional sequence at the 5′ ends to introduce Illumina read and 2 sequences. Further, they contain a 7 nt randomer sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina Miseq according to the manufacturer's instructions. Resultant sequencing reads were aligned to a reference sequence and analyzed for indels. Samples with editing that did not align to the estimated cut location or with unexpected alleles in the spacer region were discarded.

### Results

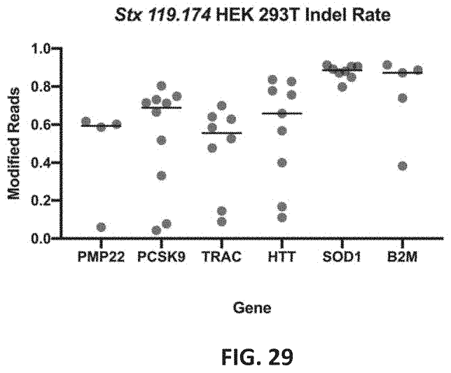

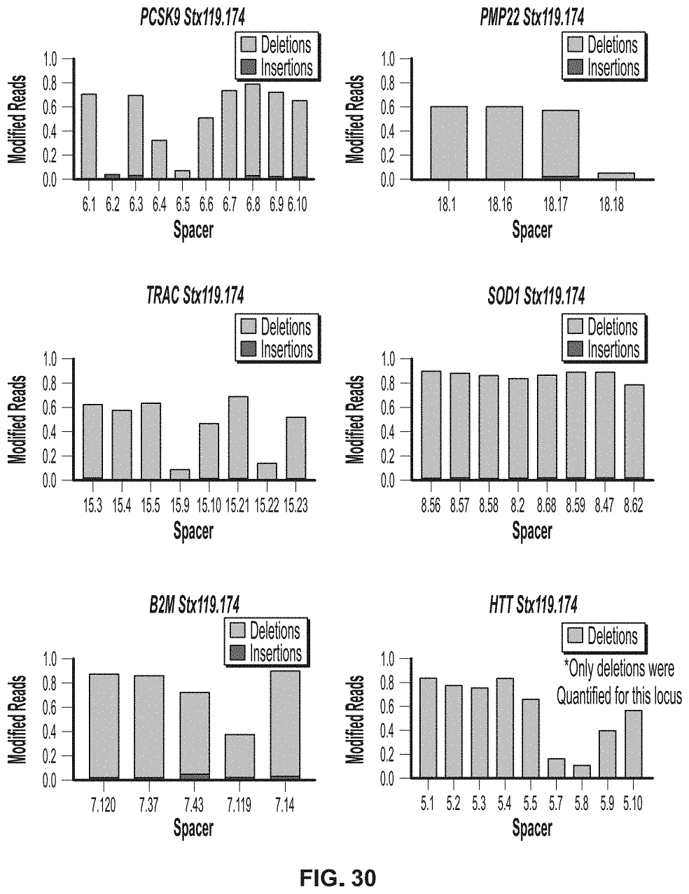

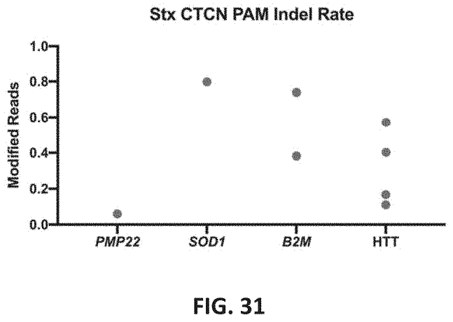

In order to validate the editing effected by the CasX:gNA 119.174 at a variety of genetic loci, a clonal plasmid transfection experiment was performed in HEK 293T cells. Multiple spacers (Table 31) were designed and cloned into an expression plasmid encoding the CasX 119 nuclease and guide 174 scaffold. HEK 293T cells were transfected with plasmid DNA, selected with puromycin, and harvested for genomic DNA six days post-transfection. Genomic DNA was analyzed via next generation sequencing (NGS) and aligned to a reference DNA sequence for analysis of insertions or deletions (indels). CasX:gNA 119.174 was able to efficiently generate indels across the 6 target genes, as shown in FIGS. 29 and 30. Indel rates varied between spacers, but median editing rates were consistently at 60% or higher, and in some cases, indel rates as high as 91% were observed. Additionally, spacers with non-canonical CTC PAMs were demonstrated to be able to generate indels with all tested target genes (FIG. 31).

The results demonstrate that the CasX variant 119 and gNA variant 174 can consistently and efficiently generate indels at a wide variety of genetic loci in human cells. The unbiased selection of many of the spacers used in the assays shows the overall effectiveness of the 119.174 RNP molecules to edit genetic loci, while the ability to target to spacers with both a TTC and a CTC PAM demonstrates its increased versatility compared to reference CasX that edit only with the TTC PAM.

TABLE 31
Spacer sequences targeting each genetic locus.

| | | | | SEQ |
|---|---|---|---|---|
| | | | | ID |
| Gene | Spacer | PAM | Spacer Sequence | NO |
| PCSK9 | 6.1 | TTC | GAGGAGGACGGCCTGGCCGA | 4061 |
| PCSK9 | 6.2 | TTC | ACCGCTGCGCCAAGGTGCGG | 4062 |
| PCSK9 | 6.4 | TTC | GCCAGGCCGTCCTCCTCGGA | 4063 |
| PCSK9 | 6.5 | TTC | GTGCTCGGGTGCTTCGGCCA | 4064 |
| PCSK9 | 6.3 | TTC | ATGGCCTTCTTCCTGGCTTC | 4065 |
| PCSK9 | 6.6 | TTC | GCACCACCACGTAGGTGCCA | 4066 |
| PCSK9 | 6.7 | TTC | TCCTGGCTTCCTGGTGAAGA | 4067 |
| PCSK9 | 6.8 | TTC | TGGCTTCCTGGTGAAGATGA | 4068 |
| PCSK9 | 6.9 | TTC | CCAGGAAGCCAGGAAGAAG | 4069 |
| | | | G | |
| PCSK9 | 6.10 | TTC | TCCTTGCATGGGGCCAGGAT | 4070 |
| PMP22 | 18.16 | TTC | GGCGGCAAGTTCTGCTCAGC | 4071 |
| PMP22 | 18.17 | TTC | TCTCCACGATCGTCAGCGTG | 4072 |
| PMP22 | 18.18 | CTC | ACGATCGTCAGCGTGAGTGC | 4073 |
| PMP22 | 18.1 | TTC | CTCTAGCAATGGATCGTGGG | 4074 |
| TRAC | 15.3 | TTC | CAAACAAATGTGTCACAAAG | 4075 |
| TRAC | 15.4 | TTC | GATGTGTATATCACAGACAA | 4076 |
| TRAC | 15.5 | TTC | GGAATAATGCTGTTGTTGAA | 4077 |
| TRAC | 15.9 | TTC | AAATCCAGTGACAAGTCTGT | 4078 |
| TRAC | 15.10 | TTC | AGGCCACAGCACTGTTGCTC | 4079 |
| TRAC | 15.21 | TTC | AGAAGACACCTTCTTCCCCA | 4080 |
| TRAC | 15.22 | TTC | TCCCCAGCCCAGGTAAGGGC | 4081 |
| TRAC | 15.23 | TTC | CCAGCCCAGGTAAGGGCAGC | 4082 |
| HTT | 5.1 | TTC | AGTCCCTCAAGTCCTTCCAG | 4083 |
| HTT | 5.2 | TTC | AGCAGCAGCAGCAGCAGCA | 4084 |
| | | | G | |
| HTT | 5.3 | TTC | TCAGCCGCCGCCGCAGGCAC | 4085 |
| HTT | 5.4 | TTC | AGGGTCGCCATGGCGGTCTC | 4086 |
| HTT | 5.5 | TTC | TCAGCTTTTCCAGGGTCGCC | 4087 |
| HTT | 5.7 | CTC | GCCGCAGCCGCCCCCGCCGC | 4088 |
| HTT | 5.8 | CTC | GCCACAGCCGGGCCGGGTGG | 4089 |
| HTT | 5.9 | CTC | TCAGCCACAGCCGGGCCGGG | 4090 |
| HTT | 5.10 | CTC | CGGTCGGTGCAGCGGCTCCT | 4091 |
| SOD1 | 8.56 | TTC | CCACACCTTCACTGGTCCAT | 4092 |
| SOD1 | 8.57 | TTC | TAAAGGAAAGTAATGGACCA | 4093 |
| SOD1 | 8.58 | TTC | CTGGTCCATTACTTTCCTTT | 4094 |
| SOD1 | 8.2 | TTC | ATGTTCATGAGTTTGGAGAT | 4095 |
| SOD1 | 8.68 | TTC | TGAGTTTGGAGATAATACAG | 4096 |
| SOD1 | 8.59 | TTC | ATAGACACATCGGCCACACC | 4097 |
| SOD1 | 8.47 | TTC | TTATTAGGCATGTTGGAGAC | 4098 |
| SOD1 | 8.62 | CTC | CAGGAGACCATTGCATCATT | 4099 |
| B2M | 7.120 | TTC | GGCCTGGAGGCTATCCAGCG | 4100 |
| B2M | 7.37 | TTC | GGCCGAGATGTCTCGCTCCG | 27 |
| B2M | 7.43 | CTC | AGGCCAGAAAGAGAGAGTA | 28 |
| | | | G | |
| B2M | 7.119 | CTC | CGCTGGATAGCCTCCAGGCC | 4101 |
| B2M | 7.14 | TTC | TGAAGCTGACAGCATTCGGG | 25 |

### Example 16: Design and Evaluation of Improved CasX Variants by Deep Mutational Evolution

The purpose of the experiments was to identify and engineer novel CasX variant proteins with enhanced genome editing efficiency relative to wild-type CasX. To cleave DNA efficiently in living cells, the CasX protein must efficiently perform the following functions: i) form and stabilize the R-loop structure consisting of a targeting guide RNA annealed to a complementary genomic target site in a DNA:RNA hybrid; and ii) position an active nuclease domain to cleave both strands of the DNA at the target sequence. These two functions can each be enhanced by altering the biochemical or structural properties of the protein, specifically by introducing amino acid mutations or exchanging protein domains in an additive or combinatorial fashion.

To construct CasX variant proteins with improved properties, an overall approach was chosen in which bacterial assays and hypothesis-driven approaches were first used to identify candidate mutations to enhance particular functions, after which increasingly stringent human genome editing assays were used in a stepwise manner to rationally combine cooperatively function-enhancing mutations in order to identify CasX variants with enhanced editing properties.

### Materials and Methods:

### Cloning and Media

Restriction enzymes, PCR reagents, and cloning strains of E. coli were obtained from New England Biolabs. All molecular biology and cloning procedures were performed according to the manufacturer's instructions. PCR was performed using Q5 polymerase unless otherwise specified. All bacterial culture growth was performed in 2XYT media (Teknova) unless otherwise specified. Standard plasmid cloning was performed in Turbo® E. coli unless otherwise specified. Standard final concentrations of the following antibiotics were used where indicated: carbenicillin: 100 μg/mL; kanamycin: 60 μg/mL; chloramphenicol: 25 μg/mL.

### Molecular Biology of Protein Library Construction

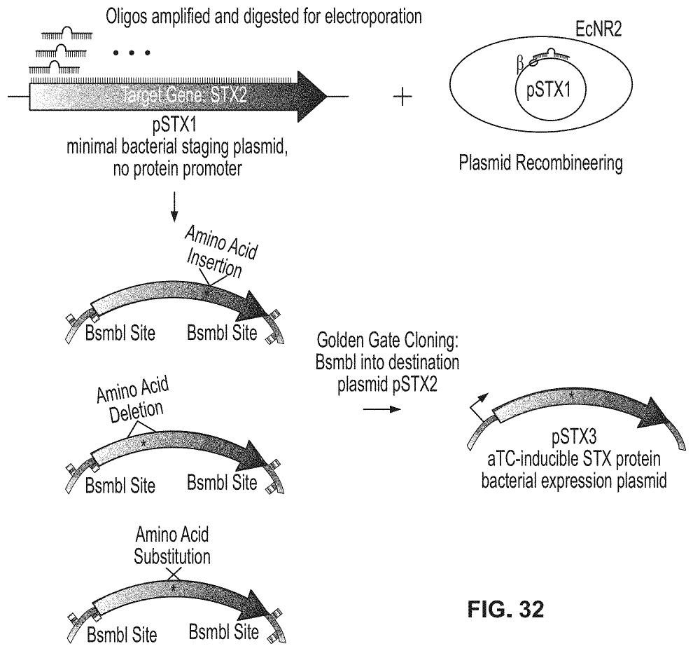

Four libraries of CasX variant proteins were constructed using plasmid recombineering in E. coli strain EcNR2 (Addgene ID: 26931), and the overall approach to protein mutagenesis was termed Deep Mutational Evolution (DME), which is schematically shown in FIG. 32. Three libraries were constructed corresponding to each of three cleavage-inactivating mutations made to the reference CasX protein open reading frame of Planctomycetes, SEQ 1D NO:2 (“STX2”), rendering the CasX catalytically dead (dCasX). These three mutations are referred to as D1 (with a D659A substitution), D2 (with a E756A substitution), or D3 (with a D922A substitution). A fourth library was composed of all three mutations in combination, referred to as DDD (D659A; E756A; D922A substitutions). These libraries were constructed by introducing desired mutations to each of the four starting plasmids. Briefly, an oligonucleotide library was obtained from Twist Biosciences and prepared for recombineering (see below). A final volume of 50 μL of 1 μM oligonucleotides, plus 10 ng of pSTX1 encoding the dCasX open reading frame (composed of either D1, D2, or D3) was electroporated into 50 μL of induced, washed, and concentrated EcNR2 using a 1 mm electroporation cuvette (BioRad GenePulser). A Harvard Apparatus ECM 630 Electroporation System was used with settings 1800 kV, 200 Ω, 25 μF. Three replicate electroporations were performed, then individually allowed to recover at 30° C. for 2 hr in 1 mL of SOC (Teknova) without antibiotic. These recovered cultures were titered on LB plates with kanamycin to determine the library size. 2XYT media and kanamycin was then added to a final volume of 6 mL and grown for a further 16 hours at 30° C. Cultures were miniprepped (QIAprep Spin Miniprep Kit) and the three replicates were then combined, completing a round of plasmid recombineering. A second round of recombineering was then performed, using the resulting miniprepped plasmid from round 1 as the input plasmid.

Oligo library synthesis and maturation: A total of 57751 unique oligonucleotide sequences designed to result in either amino acid insertion, substitution, or deletion at each codon position along the STX 2 open reading frame were synthesized by Twist Biosciences, among which were included so-called ‘recombineering oligos’ that included one codon to represent each of the twenty standard amino acids and codons with flanking homology when encoded in the plasmid pSTX1. The oligo library included flanking 5′ and 3′ constant regions used for PCR amplification. Compatible PCR primers include oSH7: 5′AACACGTCCGTCCTAGAACT (SEQ ID NO: 4102; universal forward) and oSH8: 5′ACTTGGTTACGCTCAACACT (SEQ ID NO: 4103; universal reverse) (see reference table). The entire oligo pool was amplified as 400 individual 100 μL reactions. The protocol was optimized to produce a clean band at 164 bp. Finally, amplified oligos were digested with a restriction enzyme (to remove primer annealing sites, which would otherwise form scars during recombineering), and then cleaned, for example, with a PCR clean-up kit (to remove excess salts that may interfere with the electroporation step). Here, a 600 μL final volume BsaI restriction digest was performed, with 30 μg DNA+30 μL BsaI enzyme, which was digested for two hours at 37° C.

For DME1: after two rounds of recombineering were completed, plasmid libraries were cloned into a bacterial expression plasmid, pSTX2. This was accomplished using a BsmbI Golden Gate Cloning approach to subclone the library of STX genes into an expression compatible context, resulting in plasmid pSTX3. Libraries were transformed into Turbo® E. coli (New England Biolabs) and grown in chloramphenicol for 16 hours at 37° C., followed by miniprep the next day.

For DME2: protein libraries from DME1 were further cloned to generate a new set of three libraries for further screening and analysis. All subcloning and PCR was accomplished within the context of plasmid pSTX1. Library D1 was discontinued and libraries D2 and D3 were kept the same. A new library, DDD, was generated from libraries D2 and D3 as follows. First, libraries D2 and D3 were PCR amplified such that the Dead 1 mutation, E756A, was added to all plasmids in each library, followed by blunt ligation, transformation, and miniprep, resulting in library A (D1+D2) and library B (D1+D3). Next, another round of PCR was performed to add either mutation D3 or D2, respectively, to library A and B, generating PCR products A′ and B′. At this point, A′ and B′ were combined in equimolar amounts, then blunt ligated, transformed, and miniprepped to generate a new library, DDD, containing all three dead mutations in each plasmid.

### Bacterial CRISPR Interference (CRISPRi) Screen

A dual-color fluorescence reporter screen was implemented, using monomeric Red Fluorescent Protein (mRFP) and Superfolder Green Fluorescent Protein (sfGFP), based on Qi L S, et al. Cell 152:1173-1183 (2013). This screen was utilized to assay gene-specific transcriptional repression mediated by programmable DNA binding of the CasX system. This strain of E. coli expresses bright green and red fluorescence under standard culturing conditions or when grown as colonies on agar plates. Under a CRISPRi system, the CasX protein is expressed from an anhydrotetracycline (aTc)-inducible promoter on a plasmid containing a p15A replication origin (plasmid pSTX3; chloramphenicol resistant), and the sgRNA is expressed from a minimal constitutive promoter on a plasmid containing a ColE1 replication origin (pSTX4, non-targeting spacer, or pSTX5, GFP-targeting spacer #1; carbenicillin resistant). When the CRISPRi E. coli strain is co-transformed with both plasmids, genes targeted by the spacer in pSTX4 are repressed; in this case GFP repression is observed, the degree to which is dependent on the function of the targeting CasX protein and sgRNA. In this system, RFP fluorescence can serve as a normalizing control. Specifically, RFP fluorescence is unaltered and independent of functional CasX based CRISPRi activity. CRISPRi activity can be tuned in this system by regulating the expression of the CasX protein; here, all assays used an induction concentration of 20 nM aTc final concentration in growth media.

Libraries of CasX protein were initially screened using the above CRISPRi system. After co-transformation and recovery, libraries were either: 1) plated on LB agar plus appropriate antibiotics and titered such that individual colonies could be picked, or 2) grown for eight hours in 2XYT media with appropriate antibiotics and sorted on a MA900 flow cytometry instrument (Sony). Variants of interest were detected using either standard Sanger sequencing of picked colonies (UC Berkeley Barker Sequencing Facility) or NGS sequencing of miniprepped plasmid (Massachusetts General Hospital CCIB DNA Core Next-Generation Sequencing Service).

Plasmids were miniprepped and the protein sequence was PCR-amplified, then tagmented using a Nextera kit (Illumina) to fragment the amplicon and introduce indexing adapters for sequencing on a 150 paired end HiSeq 2500 (UC Berkeley Genomics Sequencing Lab).

Bacterial ccdB Plasmid Clearance Selection

A dual-plasmid selection system was used to assay clearance of a toxic plasmid by CasX DNA cleavage. Briefly, the arabinose-inducible plasmid pBLO63.3 expressing toxic protein ccdB results in death when transformed into E. coli strain BW25113 and grown under permissive conditions. However, growth is rescued if the plasmid is cleared successfully by dsDNA cleavage, and in particular by plasmid pSTX3 co-expressing CasX protein and a guide RNA targeting the plasmid pBLO63.3. CasX protein libraries from DME1, without the catalytically inactivating mutations D1, D2, or D3, were subcloned to plasmid pSTX3. These plasmid libraries were transformed into BW25113 carrying pBLO63.3 by electroporation (200 ng of plasmid into 50 uL of electrocompetent cells) and allowed to recover in 2 mL of SOC media at 37° C. at 200 rpm shaking for 25 minutes, after which luL of 1M IPTG was added. Growth was continued for an additional 40 minutes, after which cultures were evenly divided across a 96-well deep-well block and grown in selective media for 4.5 hrs at 37° C. or 45° C. at 750 rpm. Selective media consists of the following: 2XYT with chloramphenicol+10 mM arabinose+500 μM IPTG+2 nM aTc (concentrations final). Following growth, plasmids were miniprepped to complete one round of selection, and the resulting DNA was used as input for a subsequent round. Seven rounds of selection were performed on CasX protein libraries. CasX variant Sanger sequencing or NGS was performed as described above.

### NGS Data Analysis

Paired end reads were trimmed for adapter sequences with cutadapt (version 2.1), and aligned to the reference with bowtie2 (v2.3.4.3). The reference was the entire amplicon sequence prior to tagmentation in the Nextera protocol. Each catalytically inactive CasX variant was aligned to its respective amplicon sequence. Sequencing reads were assessed for amino acid variation from the reference sequence. In short, the read sequence and aligned reference sequence were translated (in frame), then realigned and amino acid variants were called. Reads with poor alignment or high error rates were discarded (mapq <20 and estimated error rate >4%; Estimated error rate was calculated using per-base phred quality scores). Mutations at locations of poor-quality sequencing were discarded (phred score <20). Mutations were labeled for being single substitutions, insertions, or deletions, or other higher-order mutations, or outside the protein-coding sequence of the amplicon. The number of reads that supported each set of mutations was determined. These read counts were normalized for sequencing depth (mean normalization), and read counts from technical replicates were averaged by taking the geometric mean. Enrichment was calculated within each CasX variant by averaging the enrichment for each gate.

### Molecular Biology of Variants

In order to screen variants of interest, individual variants were constructed using standard molecular biology techniques. All mutations were built on STX2 using a staging vector and Gibson cloning. To build single mutations, universal forward (5′→3′) and reverse (3′→5′) primers were designed on either end of the protein sequence that had homology to the desired backbone for screening (see Table 32). Primers to create the desired mutations were also designed (F primer and its reverse complement) and used with the universal F and R primers for amplification, thus producing two fragments. In order to add multiple mutations, additional primers with overlap were designed and more PCR fragments were produced. For example, to construct a triple mutant, four sets of F/R primers were designed. The resulting PCR fragments were gel extracted and the screening vector was digested with the appropriate restriction enzymes then gel extracted. The insert fragments and vector were then assembled using Gibson assembly master mix, transformed, and plated using appropriate LB agar+antibiotic. The clones were Sanger sequenced and correct clones were chosen.

Finally, spacer cloning was performed to target the guide RNA to a gene of interest in the appropriate assay or screen. The sequence verified non-targeting clone was digested with the appropriate golden gate enzyme and cleaned using DNA Clean and Concentrator kit (Zymo). The oligos for the spacer of interest were annealed. The annealed spacer was ligated into digested and cleaned vector using a standard Golden Gate Cloning protocol. The reaction was transformed and plated on LB agar+antibiotic. The clones were sanger sequenced and correct clones were chosen.

TABLE 32
Primer sequences

| Screening | | |
|---|---|---|
| vector | F primer sequence | R primer sequence |
| pSTX6 | SAH24: | SAH25: |
| | TTCAGGTTGGACCGGTGCCACCATGGCC | TTTTGGACTAGTCACGGCGGGC |
| | CCAAAGAAGAAGCGGAAGGTCAGCCAAG | TTCCAG (SEQ ID NO: |
| | AGATCAAGAGAATCAACAAGATCAGA | 4105) |
| | (SEQ ID NO: 4104) | |
| pSTX16 or | oIC539: | oIC540: |
| pSTX34 | ATGGCCCCAAAGAAGAAGCGGAAGGTCT | TACCTTTCTCTTCTTTTTTGGA |
| | CTAGACAAG (SEQ ID NO: 4106) | CTAGTCACGG (SEQ ID NO: |
| | | 4107) |

### GFP Editing by Plasmid Lipofection of HEK293T Cells

Either doxycycline inducible GFP (iGFP) reporter HEK293T cells or SOD1-GFP reporter HEK293T cells were seeded at 20-40k cells/well in a 96 well plate in 100 μl of FB medium and cultured in a 37° C. incubator with 5% CO2. The following day, confluence of seeded cells was checked. Cells were ˜75% confluent at time of transfection. Each CasX construct was transfected at 100-500 ng per well using Lipofectamine 3000 following the manufacturer's protocol, into 3 wells per construct as replicates. SaCas9 and SpyCas9 targeting the appropriate gene were used as benchmarking controls. For each Cas protein type, a non-targeting plasmid was used as a negative control. After 24-48 hours of puromycin selection at 0.3-3 μg/ml to select for successfully transfected cells, followed by 1-7 days of recovery in FB medium, GFP fluorescence in transfected cells was analyzed via flow cytometry. In this process, cells were gated for the appropriate forward and side scatter, selected for single cells and then gated for reporter expression (Attune Nxt Flow Cytometer, Thermo Fisher Scientific) to quantify the expression levels of fluorophores. At least 10,000 events were collected for each sample. The data were then used to calculate the percentage of edited cells.

### GFP Editing by Lentivirus Transduction of HEK293T Cells

Lentivirus products of plasmids encoding CasX proteins, including controls, CasX variants, and/or CasX libraries, were generated in a Lenti-X 293T Cell Line (Takara) following standard molecular biology and tissue culture techniques. Either iGFP HEK293T cells or SOD1-GFP reporter HEK293T cells were transduced using lentivirus based on standard tissue culture techniques. Selection and fluorescence analysis was performed as described above, except the recovery time post-selection was 5-21 days. For Fluorescence-Activated Cell Sorting (FACS), cells were gated as described above on a MA900 instrument (Sony). Genomic DNA was extracted by QuickExtract™ DNA Extraction Solution (Lucigen) or Genomic DNA Clean & Concentrator (Zymo).

### Engineering of CasX Protein 2 to CasX 119

Prior work had demonstrated that CasX RNP complexes composed of functional wild-type CasX protein from Planctomycetes (hereafter referred to as CasX protein 2 {or STX2, or STX protein 2, SEQ ID NO:2} and CasX sgRNA 1 {or STX sgRNA 1, SEQ ID NO:4}) are capable of inducing dsDNA cleavage and gene editing of mammalian genomes (Liu, J J et al Nature, 566, 218-223 (2019)). However, previous observations of cleavage efficiency were relatively low (˜30% or less), even under optimal laboratory conditions. These poor rates of genome editing are insufficient for the wild-type CasX CRISPR systems to serve as therapeutic genome-editing molecules. In order to efficiently perform genome editing, the CasX protein must effectively perform two central functions: (i) form and stabilize the R-loop, and (ii) position the nuclease domain for cleavage of both DNA strands. Under conditions in which CasX RNP can access genomic DNA, genome editing rates will be partly governed by the ability of the CasX protein to perform these functions (the other controlling component being the guide RNA). The optimization of both functions is dependent on the complex sequence-function relationship between the linear chain of amino acids encoding the CasX protein and the biochemical properties of the fully formed, cleavage competent RNP. As amino acid mutations that enhance each of these functions can be combined to cumulatively result in a highly engineered CasX protein exhibiting greatly enhanced genome editing efficiency sufficient for human therapeutics, an overall engineering approach was devised in which mutations enhancing function (i) were identified, mutations enhancing function (ii) were identified, and then rational stacking of multiple beneficial mutations would be used to construct CasX variants capable of efficient genome editing. Function (i), stabilization of the R-loop, is by itself sufficient to interfere with gene expression in living cells even in the absence of DNA nuclease activity, a phenomenon known as CRISPR interference (CRISPRi). It was determined that a bacterial CRISPRi assay would be well-suited to identifying mutations enhancing this function. Similarly, a bacterial assay testing for double-stranded DNA (dsDNA) cleavage would be capable of identifying mutations enhancing function (ii). A toxic plasmid clearance assay was chosen to serve as a bacterial selection strategy and identify relevant amino acid changes. These sets of mutations were then validated to provide an enhancement to human genome editing activity, and served as the foundation for more extensive and rational combinatorial testing across increasingly stringent assays.

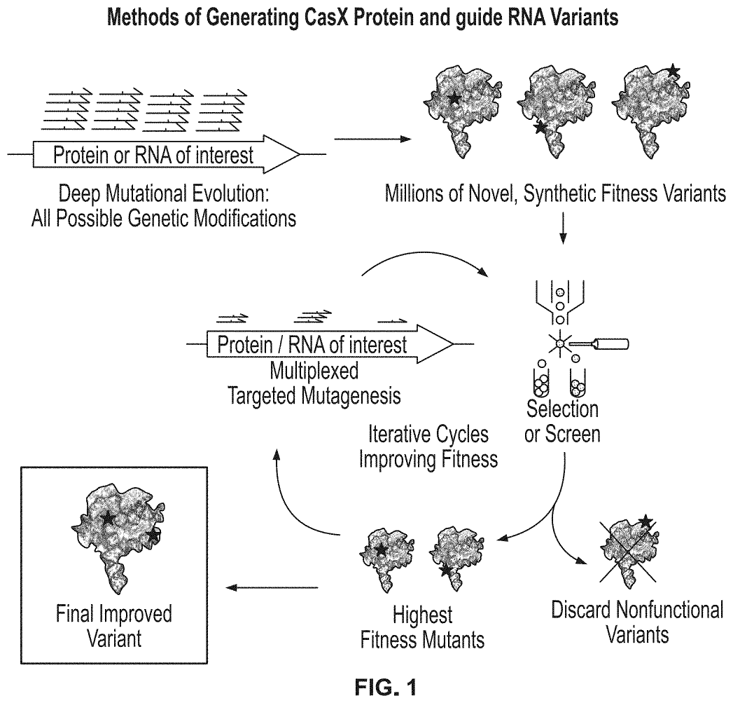

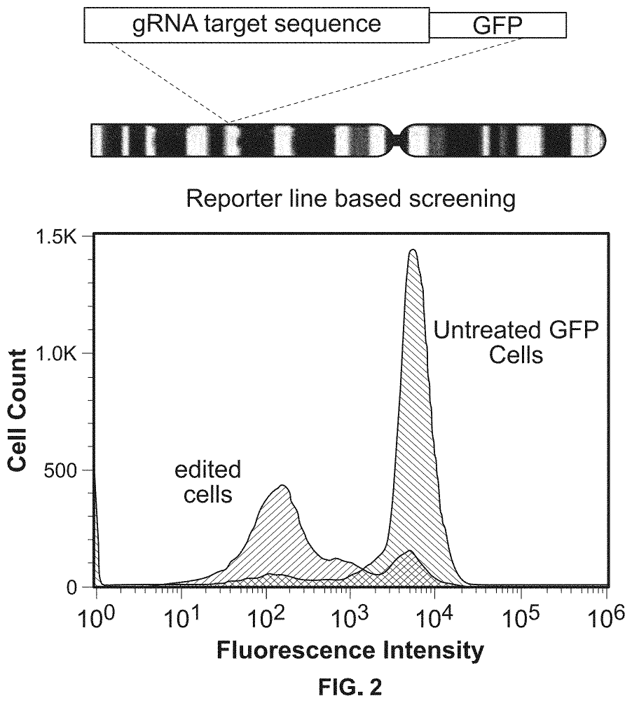

The identification of mutations enhancing core functions was performed in an engineering cycle of protein library design, molecular biology construction of libraries, and high-throughput assay of the libraries. Potential improved variants of the STX2 protein were either identified by NGS of a high-throughput biological assay, sequenced directly as clones from a population, or designed de novo for specific hypothesis testing. For high-throughput assays of functions (i) or (ii), a comprehensive and unbiased design approach to mutagenesis was desired for initial diversification. Plasmid recombineering was chosen as a sufficiently comprehensive and rapid method for library construction and was performed in a promoterless staging vector pSTX1 in order to minimize library bias throughout the cloning process. A comprehensive oligonucleotide pool encoded all possible single amino acid substitutions, insertions, and deletions in the STX2 sequence was constructed by DME; the first round of library construction and screening is hereafter referred to as DME1 (FIG. 1). While recombineering is known to produce substantially biased mutation libraries (even from initially uniform pools of oligonucleotides), we deemed this tradeoff acceptable in exchange for an accelerated experimental timeline to improved activity levels. Two high-throughput bacterial assays were chosen to identify potential improved variants from the diverse set of mutations in DME1. As discussed above, we reasoned that a CRISPRi bacterial screen would identify mutations enhancing function (i). While CRISPRi uses a catalytically inactive form of the CasX protein, many specific characteristics together influence the total enhancement of this function, such as expression efficiency, folding rate, protein stability, or stability of the R-loop (including binding affinity to the sgRNA or DNA). DME1 libraries were constructed on the dCasX mutant templates and individually screened. Screening was performed as Fluorescence-Activated Cell Sorting (FACS) of GFP repression in a previously validated dual-color CRISPRi scheme.

### Results:

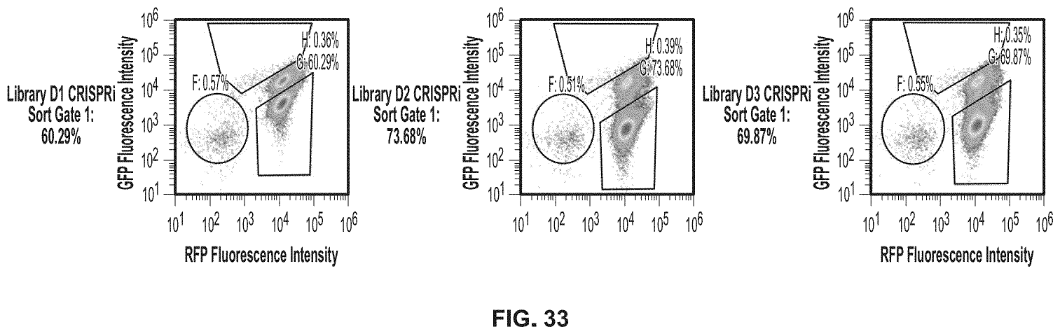

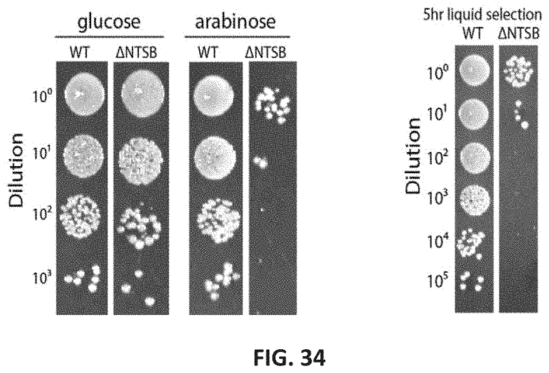

For each of DME1, DME2 and DME3, the three libraries exhibited a different baseline CRISPRi activity, thereby serving as independent, yet related, screens. For each library, gates of varying stringency were drawn around the population of interest, and sorted cell populations were deep sequenced to identify CasX mutations enhancing GFP repression (FIG. 33). A second high-throughput bacterial assay was developed to assess dsDNA cleavage in E. coli by way of selection (see methods). When this assay is performed under selective conditions, a functional STX2 RNP can exhibit ˜1000- to 10,000-fold increase in colony forming units compared to nonfunctional CasX protein (FIG. 34). Multiple rounds of liquid media selections were performed for the cleavage-competent libraries of DME1. Sequential rounds of colony picking and sequencing identified mutations to enhance function (ii). Several mutations were observed with increasing frequency with prolonged selection. One mutation of note, the deletion of proline 793, was first observed in round four at a frequency of two out of 36 sequenced colonies. After round five, the frequency increased to six out of 36 sequenced colonies. In round seven, it was observed in ten out of 48 sequenced colonies. This round-over-round enrichment suggested mutations observed in these assays could potentially enhance function (ii) of the CasX protein. Selected mutations observed across these assays can be found in Table 33 as follows:

TABLE 33
Selected mutations observed in bacterial
assays for function (i) or (ii)

| Pos. | Ref. | Alternative* | Assay |
|---|---|---|---|
| 2 | Q | R | 45 C ccdb colony |
| 72 | T | S | D2 CRISPRi |
| 80 | A | T | 37 C ccdb colony |
| 111 | R | K | 45 C ccdb colony |
| 119 | G | C | 45 C ccdb colony |
| 121 | E | D | 37 C ccdb colony |
| 153 | T | I | 37 C ccdb colony |
| 166 | R | S | D2 CRISPRi |
| 203 | R | K | 45 C ccdb colony |
| 270 | S | W | 37 C ccdb colony |
| 346 | D | Y | 45 C ccdb colony |
| 361 | D | A | D1 CRISPRi |
| 385 | E | A | D3 CRISPRi |
| 386 | E | R | 45 C ccdb colony |
| 390 | K | R | D3 CRISPRi |
| 399 | F | L | 45 C ccdb colony |
| 421 | A | G | D2 CRISPRi |
| 433 | S | N | 45 C ccdb colony |
| 489 | D | S | D3 CRISPRi |
| 536 | F | S | D3 CRISPRi |
| 546 | I | V | D2 CRISPRi |
| 552 | E | A | D3 CRISPRi |
| 591 | R | I | 37 C ccdb colony |
| 595 | E | G | D3 CRISPRi |
| 636 | A | D | D3 CRISPRi |
| 657 | — | G | DI CRISPRi |
| 661 | — | L | DI CRISPRi |
| 661 | — | A | D1 CRISPRi |
| 663 | N | S | DI CRISPRi |
| 679 | S | N | D2 CRISPRi |
| 695 | G | H | 45 C ccdb colony |
| 696 | — | P | 45 C ccdb colony |
| 707 | A | D | D3 CRISPRi |
| 708 | A | K | 45 C ccdb colony |
| 712 | D | Q | 37 C ccdb colony |
| 732 | D | P | D1 CRISPRi |
| 751 | A | S | D3 CRISPRi |
| 774 | — | G | DI CRISPRi |
| 788 | A | W | D2 CRISPRi |
| 789 | Y | T | DI CRISPRi |
| 789 | Y | D | D2 CRISPRi |
| 791 | G | M | 45 C ccdb colony |
| 792 | L | E | 45 C ccdb colony |
| 793 | P | — | 45 C ccdb colony |
| 793 | — | AS | 45 C ccdb colony |
| 793 | P | T | 45 C ccdb colony |
| 793 | P | — | DI CRISPRi |
| 793 | — | F | D2 CRISPRi |
| 794 | — | PG | 45 C ccdb colony |
| 794 | — | PS | 45 C ccdb colony |
| 795 | — | AS | 37 C ccdb colony |
| 795 | — | AS | 45 C ccdb colony |
| 796 | — | AG | 37 C ccdb colony |
| 797 | — | AS | 45 C ccdb colony |
| 797 | Y | L | 45 C ccdb colony |
| 799 | S | A | D3 CRISPRi |
| 867 | S | G | 45 C ccdb colony |
| 889 | — | L | 37 C ccdb colony |
| 897 | L | M | 45 C ccdb colony |
| 922 | D | K | Dl CRISPRi |
| 963 | Q | P | D2 CRISPRi |
| 975 | K | Q | D2 CRISPRi |

*substitution, insertion, or deletion; Pos.: Position

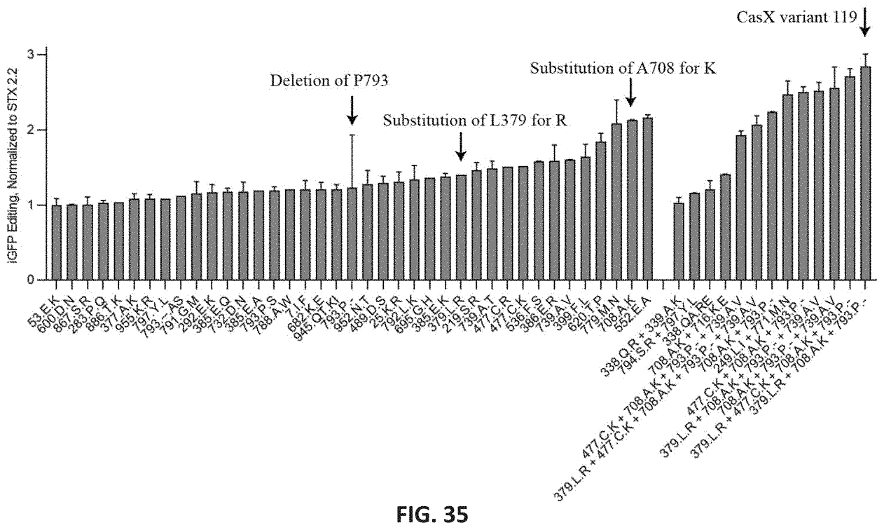

The mutations observed in the bacterial assays above were selected for their potential to enhance CasX protein functions (i) or (ii), but desirable mutations will enhance at least one function while simultaneously remaining compatible with the other. To test this, mutations were tested for their ability to improve human cell genome editing activity overall, which requires both functions acting in concert. A HEK293T GFP editing assay was implemented in which human cells containing a stably-integrated inducible GFP (iGFP) gene were transduced with a plasmid that expresses the CasX protein and sgRNA 2 with spacers to target the RNP to the GFP gene. Mutations identified in bacterial screens, bacterial selections, as well as mutations chosen de novo from biochemical hypotheses resulting from inspection of the published Cryo-EM structure of the homologous DpbCasX protein, were tested for their relative improvement to human genome editing activity as quantified relative to the parent protein STX 2 (FIG. 35), with the greatest improvement demonstrated for construct 119, shown at the bottom of FIG. 35. Several dozen of the proposed function-enhancing mutations were found to improve human cell genome editing substantially, and selected mutations from these assays can be found in Table 34 as follows:

TABLE 34
Selected single mutations observed to enhance genome editing

| | | | Fold-Improvement |
|---|---|---|---|
| | | | (average of |
| Position | Reference | Alternative* | two GFP spacers) |
| 379 | L | R | 1.4 |
| 708 | A | K | 2.13 |
| 620 | T | P | 1.84 |
| 385 | E | P | 1.19 |
| 857 | Y | R | 1.95 |
| 658 | I | V | 1.94 |
| 399 | F | L | 1.64 |
| 404 | L | K | 2.23 |
| 793 | P | — | 1.23 |
| 252 | Q | K | 1.12** |

*substitution, insertion, or deletion
**calculated as the average improvement across four variants with and without the mutation

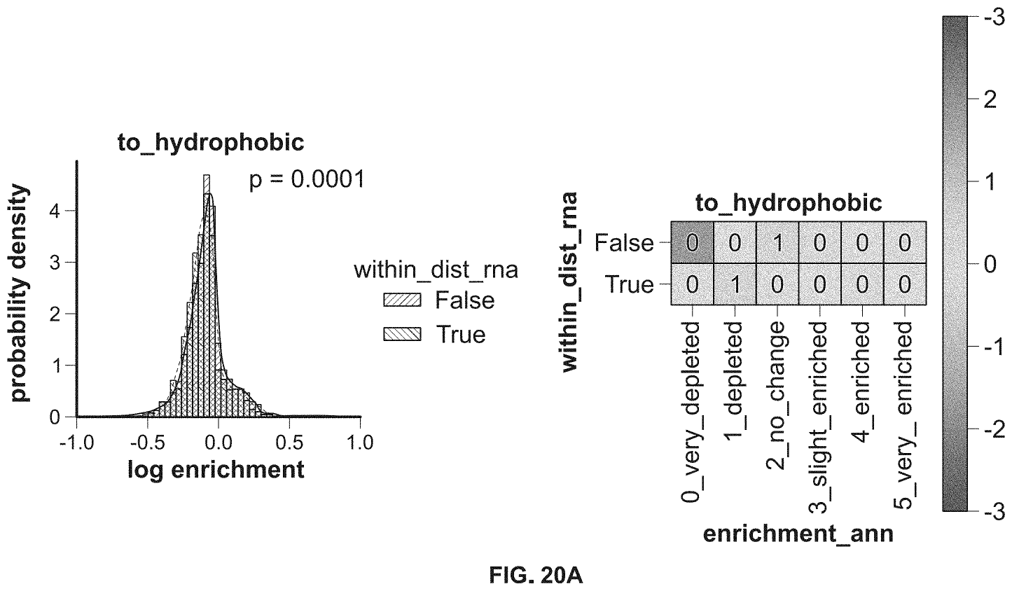

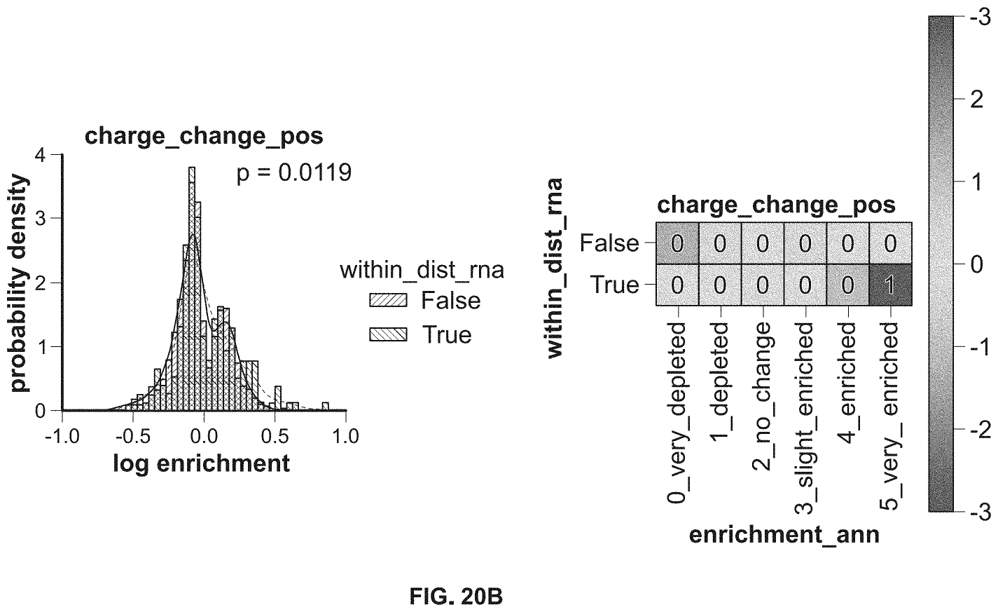

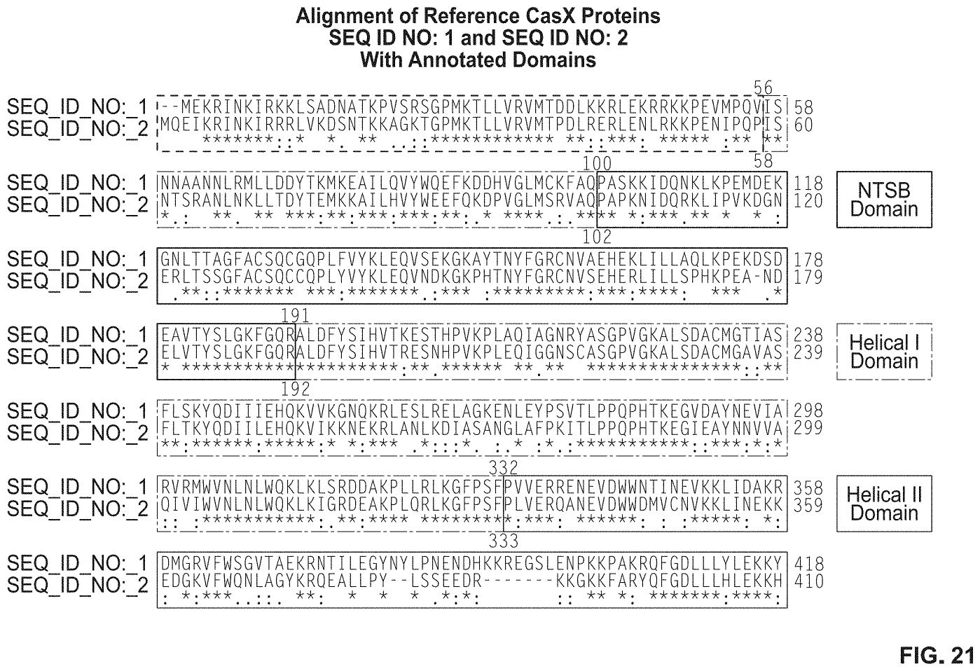

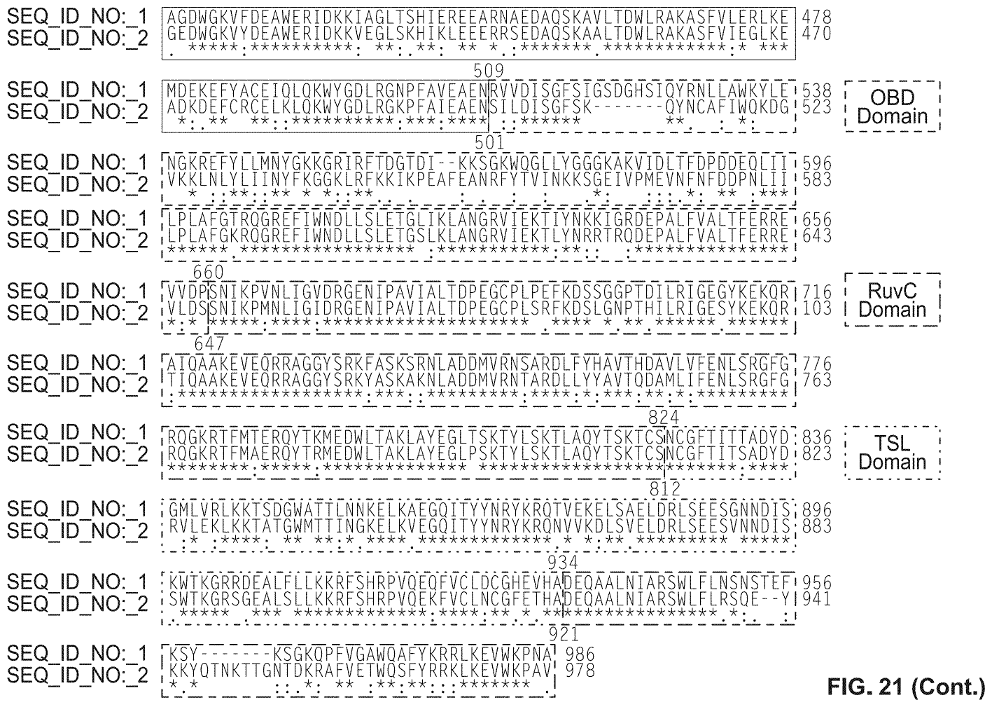

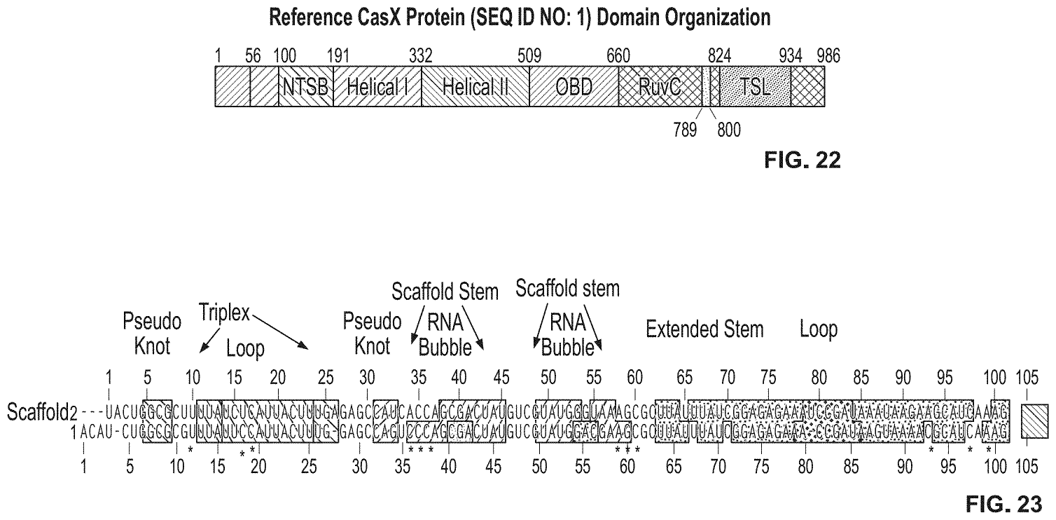

The overall engineering approach taken here relies on the central hypothesis that individual mutations enhancing each function can be additively combined to obtain greatly enhanced CasX variants with improved editing capability. FIGS. 20A-20B are a pair of plots that demonstrate that specific subsets of changes discovered by DME of the CasX are more likely to predict improvements of activity. To test this, the single mutations were first identified if they enhanced overall editing activity. Of particular note here, a substitution of the hydrophobic leucine 379 in the helical II domain to a positively charged arginine resulted in a 1.40 fold-improvement in editing activity. This mutation might provide favorable ionic interactions with the nearby phosphate backbone of the DNA target strand (between PAM-distal bp 22 and 23), thus stabilizing R-loop formation and thereby enhancing function (i). A second hydrophobic to charged mutation, alanine 708 to lysine, increased editing activity by 2.13-fold, and might provide additional ionic interactions between the RuvC domain and the sgRNA 5′ end, thus plausibly enhancing function (i) by increasing the binding affinity of the protein for the sgRNA and thereby increasing the rate of R-loop formation. The deletion of proline 793 improved editing activity by 1.23-fold by shortening a loop between an alpha helix and a beta sheet in the RuvC domain, potentially enhancing function (ii) by favorably altering nuclease positioning for dsDNA cleavage. Overall, several dozen single mutations were found to improve editing activity, including mutations identified from each of the bacterial assays as well as mutations proposed from de novo hypothesis generation. To further identify those mutations that enhanced function in a cooperative manner, rational CasX variants composed of combinations of multiple mutations were tested (FIG. 35). An initial small combinatorial set was designed and assayed, of which CasX variant 119 emerged as the overall most improved editing molecule, with a 2.8-fold improved editing efficiency compared to the STX2 wild-type protein. Variant 119 is composed of the three single mutations L379R, A708K, and [P793], demonstrating that their individual contributions to enhancement of function are additive.

### SOD1-GFP Assay Development.

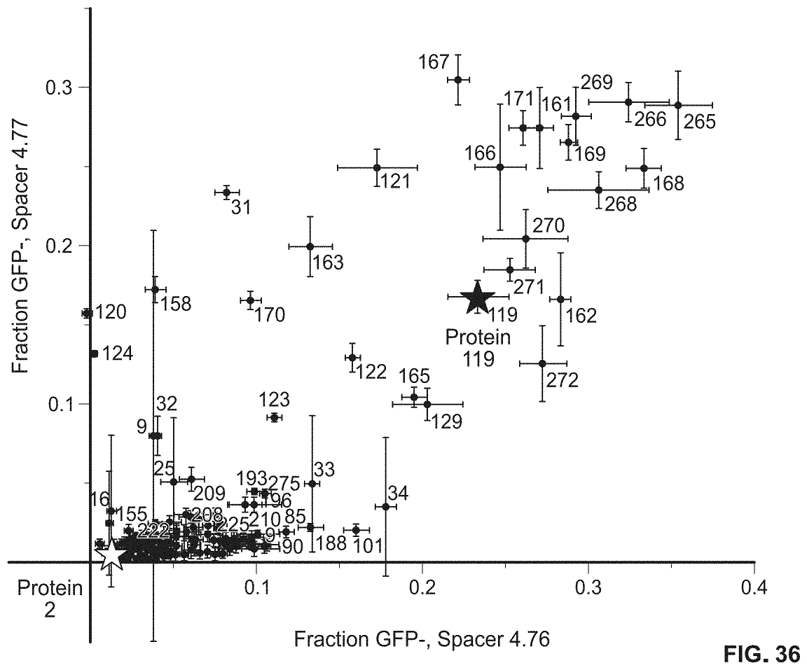

To assess CasX variants with greatly improved genome-editing activity, we sought to develop a more stringent genome editing assay. The iGFP assay provides a relatively facile editing target such that STX protein 2 in the assays above exhibited an average editing efficiency of 41% and 16% with GFP targeting spacers 4.76 and 4.77 respectively. As protein variants approach 2-fold or greater efficiency improvements, the assay becomes saturated. Therefore a new HEK293T cell line was developed with the GFP sequence integrated in-frame at the C-terminus of the endogenous human gene SOD1, termed the SOD1-GFP line. This cell line served as a new, more stringent, assay to measure the editing efficiency of several hundred additional CasX variant proteins (FIG. 36). Additional mutations were identified from bacterial assays, including a second iteration of DME library construction and screening, as well as utilizing hypothesis-driven approaches. Further exploration of combinatorial improved variants was also performed in the SOD1-GFP assay.

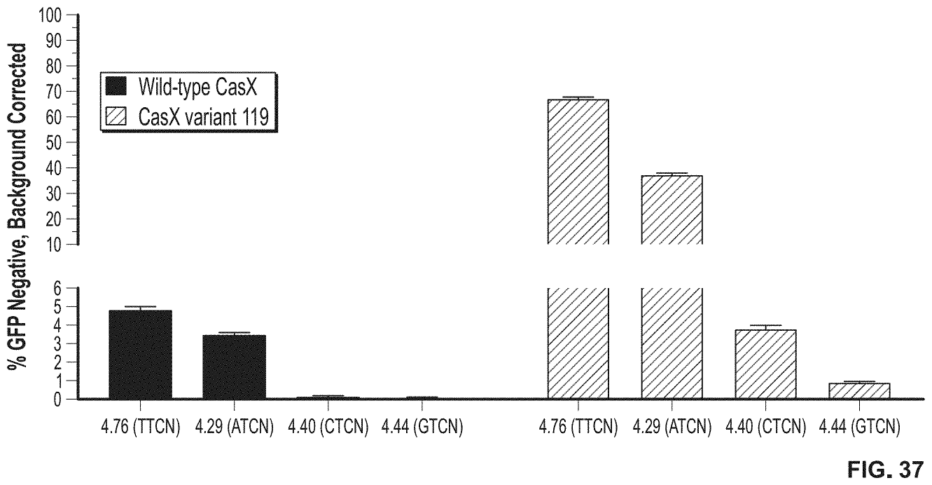

In light of the SOD1-GFP assay results, measured efficiency improvements were no longer saturated, and CasX variant 119 (indicated by the star in FIG. 36) exhibited a 23.9-fold improvement relative to the wild-type CasX (average of two spacers), with several constructs exhibiting enhanced activity relative to the CasX 119 construct. Alternatively, the dynamic range of the iGFP assay could be increased (though perhaps not completely unsaturated) by reducing the baseline activity of the WT CasX protein, namely by using sgRNA variant 1 rather than 2. Under these more stringent conditions of the iGFP assay, CasX variant 119 exhibited a 15.3-fold improvement relative to the wild-type CasX using the same spacers. Intriguingly, CasX variant 119 also exhibited substantial editing activity with spacers utilizing each of the four NTCN PAM sequences, while WT CasX only edited above 1% with spacers utilizing TTCN and ATCN PAM sequences (FIG. 37), demonstrating the ability of the CasX variant to effectively edit using an expanded spectrum of PAM sequences.

### CasX Function Enhancement by Extensive Combinatorial Mutagenesis.

Potential improved variants tested in the variety of assays above provided a dataset from which to select candidate lead proteins. Over 300 proteins were assessed in individual clonal assays and of these, 197 single mutations were assessed; the remaining ˜100 proteins contained combinatorial combinations of these mutations. Protein variants were assessed via three different assays (plasmid p6 by iGFP, plasmid p6 by SOD1-GFP, or plasmid p16 by SOD1-GFP). While single mutants led to significant improvements in the iGFP assay (with fraction GFP—greater than 50%), these single-mutants all performed poorly in the SOD1-GFP p6 backbone assay (fraction GFP—less than 10%). However, proteins containing multiple, stacked mutations were able to successfully inactivate GFP in this more stringent assay, indicating that stacking of improved mutations could substantially improve cleavage activity.

Individual mutations observed to enhance function often varied in their capacity to additively improve editing activity when combined with additional mutations. To rationally quantify these epistatic effects and further improve genome editing activity, a subset of mutations was identified that had each been added to a protein variant containing at least one other mutation, and where both proteins (with and without the mutation) were tested in the same experimental context (assay and spacer; 46 mutations total). To determine the effect due to that mutation, the fraction GFP—was compared with and without the mutation. For each protein/experimental context, the mutation effect was quantified as: 1) substantially improving the activity (fv>1.1 f0 where f0 is the fraction GFP—without the mutation, and fv is the fraction GFP—with the mutation), 2) substantially worsening the activity (fv<0.9f0), or 3) not affecting activity (neither of the other conditions are met). An overall score per mutation was calculated (s), based on the fraction of protein/experiment contexts in which the mutation substantially improved activity, minus the fraction of contexts in which the mutation substantially worsened activity. Out of the 46 mutations obtained, only 13 were associated with consistently increased activity (s≥0.5), and 18 mutations substantially decreased activity (s≤−0.5). Importantly, the distinction between these mutations was only clear when examining epistatic interactions across a variety of variant contexts: all of these mutations had comparable activity in the iGFP assay when measured alone.

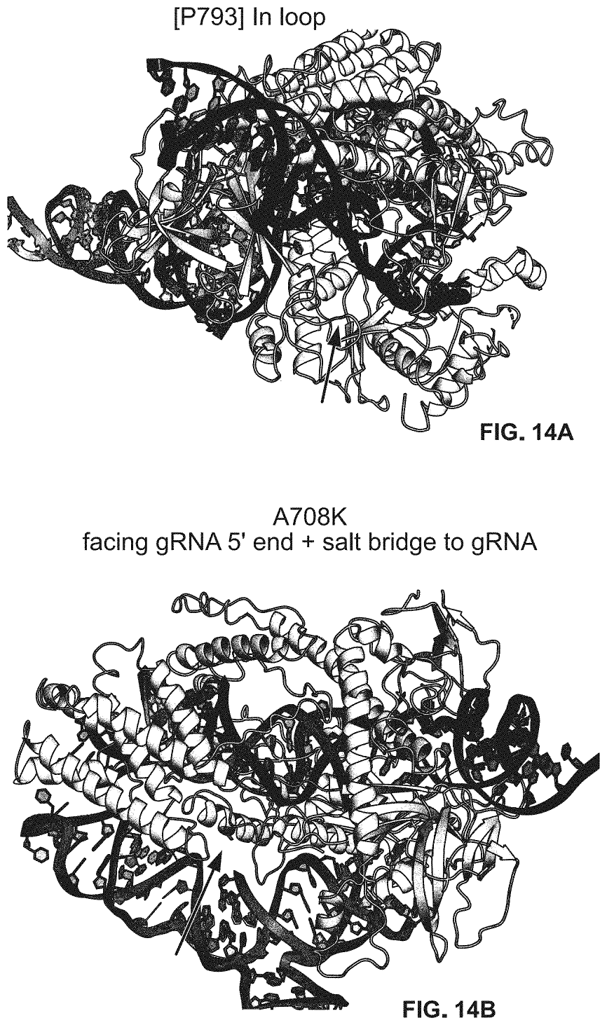

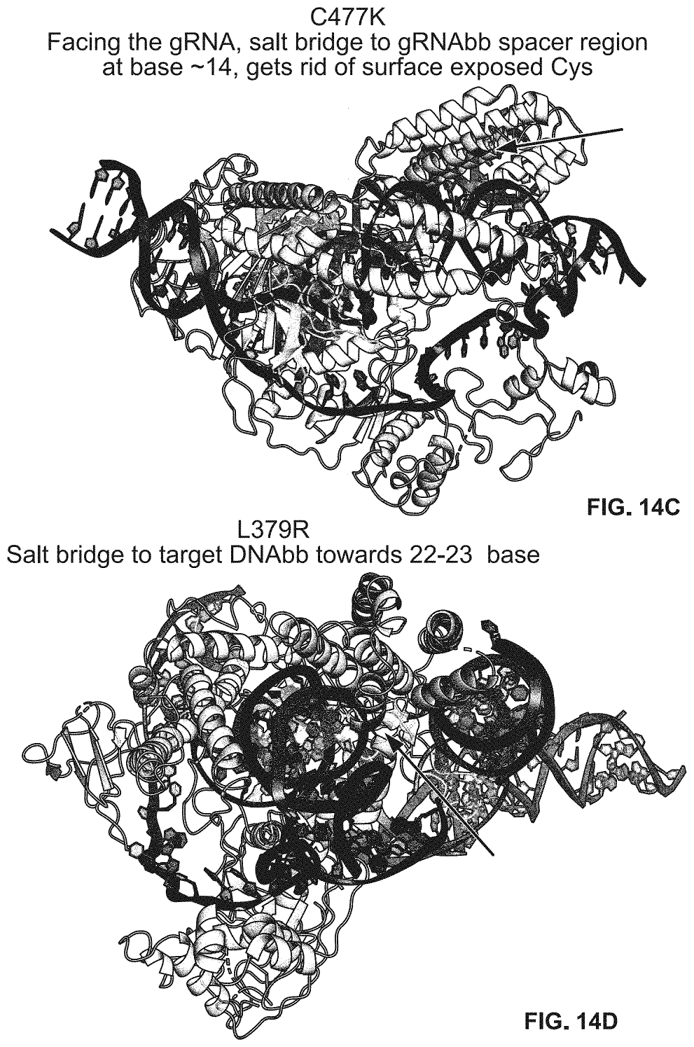

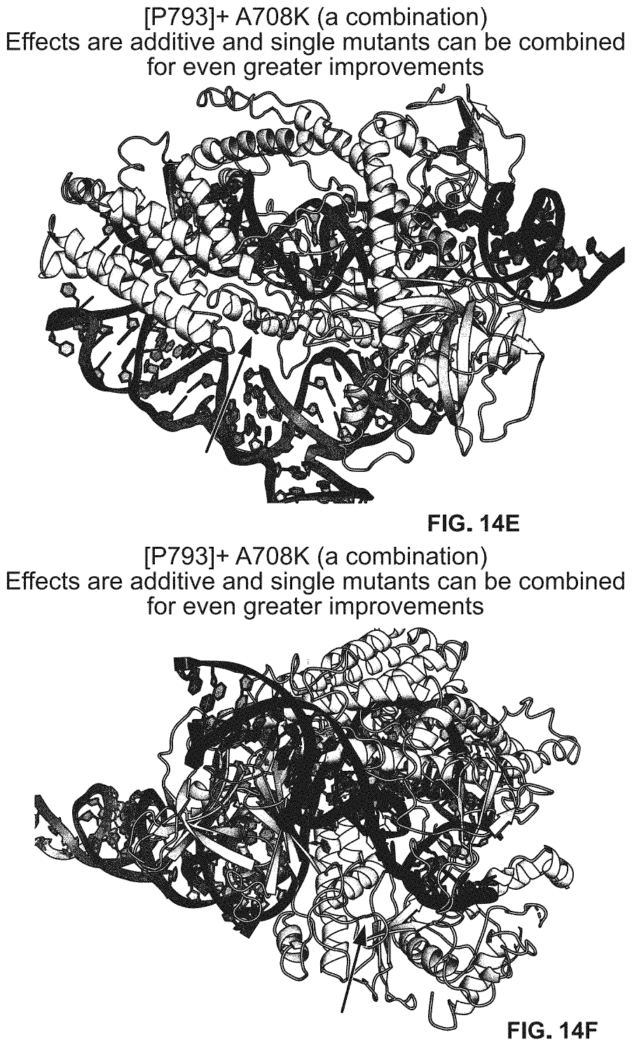

The above quantitative analysis allowed the systematic design of an additional set of highly engineered CasX proteins composed of single mutations enhancing function both individually and in combination. First, seven out of the top 13 mutations were chosen to be stacked (the other 6 variants comprised the three variants A708K, [P793] and L379R that were included in all proteins, and another two that affected redundant positions; see FIGS. 14A-14F). These mutations were iteratively stacked onto three different versions of the CasX protein: CasX 119, 311, and 365; proceeding to add only one mutation (for example, Y857R), to adding several mutations in combination. In order to maximize the combination of enhancements for both function (i) and function (ii), individual mutations were rationally chosen to maintain a diversity of biochemical properties—i.e., multiple mutations that substitute a hydrophobic residue with a negatively charged residue were avoided. The resulting ˜30 protein variants had between five and 10 individual mutations relative to STX2 (mode=7 mutations). The proteins were tested in a lipofection assay in a new backbone context (p34) with guide scaffold 64, and most showed improvement relative to protein 119. The most improved variant of this set, protein 438, was measured to be >20% improved relative to protein 119 (see Table 35 below).

Lentiviral Transduction iGFP Assay Development

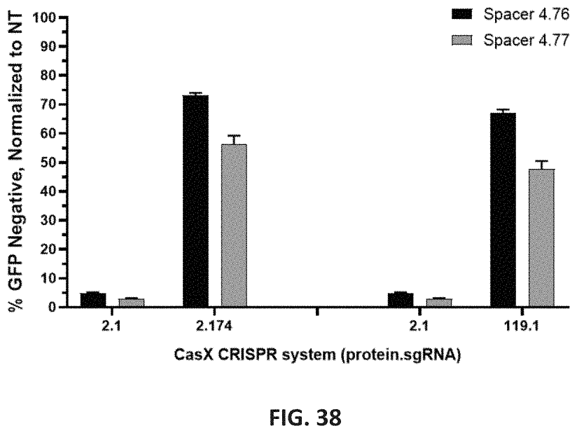

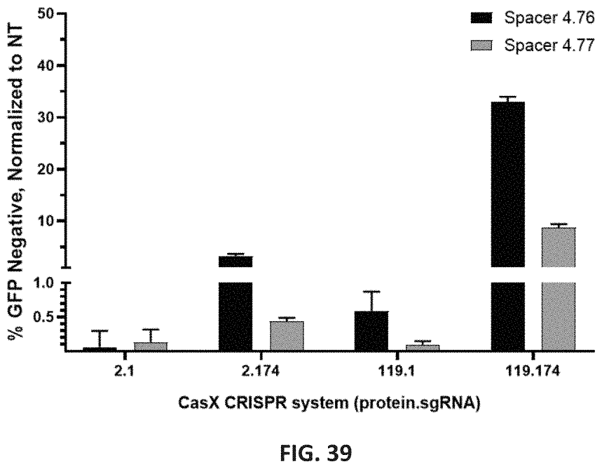

As discussed above regarding the iGFP assay, enhancements to the CasX system had likely resulted in the lipofection assay becoming saturated—that is, limited by the dynamic range of the measurement. To increase the dynamic range, a new assay was designed in which many fewer copies of the CasX gene are delivered to human cells, consisting of lentiviral transductions in a new backbone context, plasmid pSTX34. Under this more stringent delivery modality, the dynamic range was sufficient to observe the improvements of CasX variant protein 119 in the context of a further improved sgRNA, namely sgRNA variant 174. Improved variants of both the protein and sgRNA were found to additively combine to produce yet further improved CasX CRISPR systems. Protein variant 119 and sgRNA variant 174 were each measured to improve iGFP editing activity by approximately an order of magnitude when compared with wild-type CasX protein 2 (SEQ ID NO:2) in complex with sgRNA 1 (SEQ ID NO:4) under the lipofection iGFP assay (FIG. 38). Moreover, improvements to editing activity from the protein and sgRNA appear to stack nearly linearly; while individually substituting CasX 2 for CasX 119, or substituting sgRNA 174 for sgRNA 1, produces a ten-fold improvement, substituting both simultaneously produces at least another ten-fold improvement (FIG. 39). Notably, this range of activity improvements exceeds the dynamic range of either assay. However, the overall activity improvement can be estimated by calculating the fold change relative to the sample 2.174, which was measured precisely in both assays. The enhancement of the highly engineered CasX CRISPR system 119.174 over wild type CasX CRISPR system 2.1 resulted in a 259-fold improvement in genome editing efficiency in human cells (+/−58, propagated standard deviation), supporting that, under the conditions of the assay, the engineering of both the CasX and the guide led to dramatic improvements in editing efficiency compared to wild-type CasX and guide.

### Engineering of Domain Exchange Variants

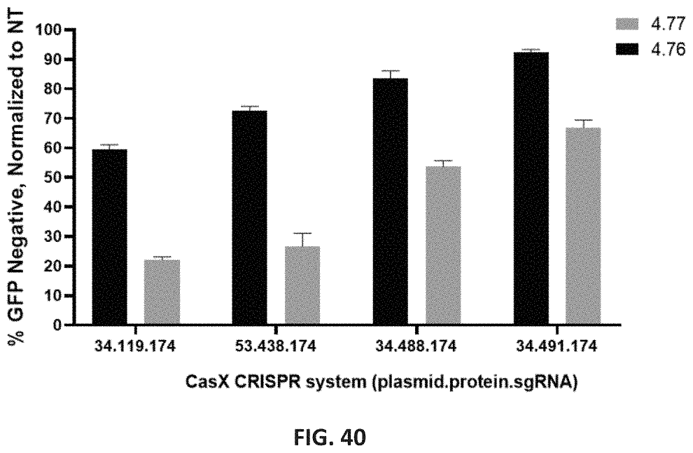

One problematic limitation of mutagenesis-based directed evolution is the combinatorial increase of possible sequences as one takes larger steps in sequence-space. To overcome this, swapping of protein domains from homologous sequences was evaluated as an alternative approach. To take advantage of the phylogenetic data available for the CasX CRISPR system, alignments were made between the CasX 1 (SEQ ID NO:1) and CasX 2 (SEQ ID NO:2) protein sequences, and domains were annotated for exchange in the context of improved CasX variant protein 119. To benchmark CasX 119 against the top designed combinatorial CasX variant proteins and the top domain exchanged variants, all within the context of improved sgRNA 174, a stringent iGFP lentiviral transduction assay was performed. Protein variants from each class were identified as improved relative to CasX variant 119 (FIG. 40), and fold changes are represented in Table 35. For example, at day 13, CasX 119.174 with GFP spacer 4.76 leads to phenotype disruption in only ˜60% of cells, while CasX variant 491 in the same context results in >90% phenotypic editing. To summarize, the compared proteins contained the following number of mutations relative to the WT CasX protein 2: 119=3 point mutations; 438 =7 point mutations; 488=protein 119, with NTSB and helical Ib domains from CasX 1 (67 mutations total); 491=5 point mutations, with NTSB and helical Ib domains from CasX 1 (69 mutations total).

TABLE 35
CasX variant improvements over CasX variant 119 in the iGFP
lentiviral transduction assay, in the context of improved sgRNA 174.

| | Fold-change | Fold-change |
|---|---|---|
| Cas X | editing activity, | editing activity, |
| Protein | spacer 4.76* | spacer 4.77* |
| 119 | 1.00 | 1.00 |
| 438 | 1.22 | 1.21 |
| 488 | 1.41 | 2.43 |
| 491 | 1.55 | 3.03 |

*relative to CasX 119

The results demonstrate that the application of rationally-designed libraries, screening, and analysis methods into a technique we have termed Deep Mutational Evolution to scan fitness landscapes of both the CasX protein and guide RNA enabled the identification and validation of mutations which enhanced specific functions, contributing to the improvement of overall genome editing activity. These datasets enabled the rational combinatorial design of further improved CasX and guide variants disclosed herein.

### Example 17: Design and Evaluation of Improved Guide RNA Variants

The existing CasX platform based on wild-type sequences for dsDNA editing in human cells achieves very low efficiency editing outcomes when compared with alternative CRISPR systems (Liu, J J et al Nature, 566, 218-223 (2019)). Cleavage efficiency of genomic DNA is governed, in large part, by the biochemical characteristics of the CasX system, which in turn arise from the sequence-function relationship of each of the two components of a cleavage-competent CasX RNP: a CasX protein complexed with a sgRNA. The purpose of the following experiments was to create and identify gRNA scaffold variants with enhanced editing properties relative to wild-type CasX:gNA RNP through a program of comprehensive mutagenesis and rational approaches.

### Methods

Methods for High-Throughput sgRNA Library Screens
1) Molecular Biology of sgRNA Library Construction

To build a library of sgRNA variants, primers were designed to systematically mutate each position encoding the reference gRNA scaffold of SEQ ID NO: 5, where mutations could be substitutions, insertions, or deletions. In the following in vivo bacterial screens for sgRNA mutations, the sgRNA (or mutants thereof) was expressed from a minimal constitutive promoter on the plasmid pSTX4. This minimal plasmid contains a ColE1 replication origin and carbenicillin antibiotic resistance cassette, and is 2311 base pairs in length, allowing standard Around-the-Horn PCR and blunt ligation cloning (using conventional methodologies). Forward primers KST223-331 and reverse primers KST332-440 tile across the sgRNA sequence in one base-pair increments and were used to amplify the vector in two sequential PCR steps. In step 1, 108 parallel PCR reactions are performed for each type of mutation, resulting in single base mutations at each designed position. Three types of mutations were generated. To generate base substitution mutations, forward and reverse primers were chosen in matching pairs beginning with KST224+KST332. To generate base insertion mutations, forward and reverse primers were chosen in matching pairs beginning with KST223+KST332. To generate base deletion mutations, forward and reverse primers were chosen in matching pairs beginning with KST225+KST332. After Step 1 PCR, samples were pooled into an equimolar manner, blunt-ligated, and transformed into Turbo E. coli (New England Biolabs), followed by plasmid extraction the next day. The resulting plasmid library theoretically contained all possible single mutations. In Step 2, this process of PCR and cloning was then repeated using the Step 1 plasmid library as the template for the second set of PCRs, arranged as above, to generate all double mutations. The single mutation library from Step 1 and the double mutation library from Step 2 were pooled together.

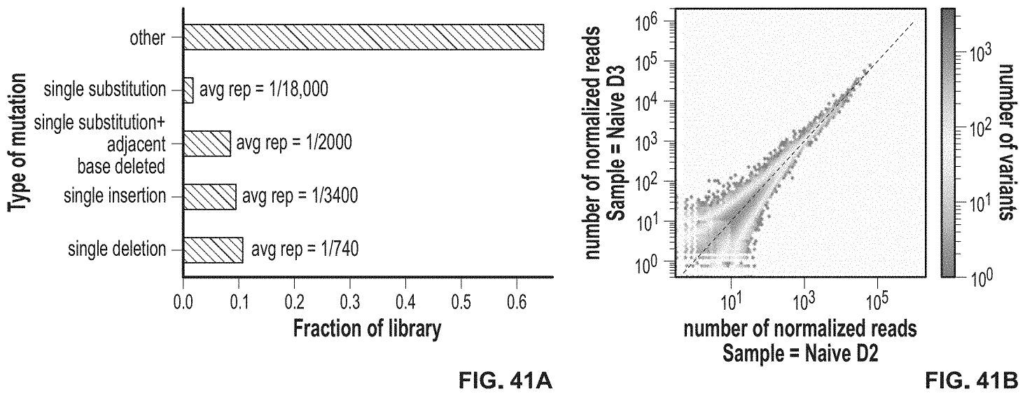

After the above cloning steps, the library diversity was assessed with next generation sequencing (see below section for methods) (see FIG. 41). It was confirmed that the majority of the library contained more than one mutation (‘other’) category. A substantial fraction of the library contained single base substitutions, deletions, and insertions (average representation within the library of 1/18,000 variants for single substitutions, and up to 1/740 variants for single deletions).

2) Assessing Library Diversity with Next Generation Sequencing.

For NGS analysis, genomic DNA was amplified via PCR with primers specific to the scaffold region of the bacterial expression vector to form a target amplicon. These primers contain additional sequence at the 5′ ends to introduce Illumina read (see Table 36 for sequences). Typical PCR conditions were: 1× Kapa Hifi buffer, 300 nM dNTPs, 300 nM each primer, 0.75 ul of Kapa Hifi Hotstart DNA polymerase in a 50 μl reaction. On a thermal cycler, incubate for 95° C. for 5 min; then 16-25 cycles of 98° C. for 15 s, 60° C. for 20 s, 72° C. for 1 min; with a final extension of 2 min at 72° C. Amplified DNA product was purified with Ampure XP DNA cleanup kit, with elution in 30 μl of water. A second PCR step was done with indexing adapters to allow multiplexing on the Illumina platform. 20 μl of the purified product from the previous step was combined with 1× Kapa GC buffer, 300 nM dNTPs, 200 nM each primer, 0.75 of Kapa Hifi Hotstart DNA polymerase in a 50 μl reaction. On a thermal cycler, cycle for 95° C. for 5 min; then 18 cycles of 98° C. for 15 s, 65° C. for 15 s, 72° C. for 30 s: with a final extension of 2 min at 72° C. Amplified DNA product was purified with Ampure XP DNA cleanup kit, with elution in 30 μl of water. Quality and quantification of the amplicon was assessed using a Fragment Analyzer DNA analyzer kit (Agilent, dsDNA 35-1500 bp).

TABLE 36
primer sequences.

| Primer | SEQ ID NO |
|---|---|
| PCR1 Fwd | 4108 |
| PCR2 Rvs | 4109 |
| PCR2 Fwd | 4110 |
| PCR2_Rvs_v1_001 | 4111 |
| PCR2_Rvs_v1_002 | 4112 |
| PCR2_Rvs_v1_003 | 4113 |
| PCR2_Rvs_v1_004 | 4114 |
| PCR2_Rvs_v1_005 | 4115 |
| PCR2_Rvs_v1_006 | 4116 |
| PCR2_Rvs_v1_007 | 4117 |
| PCR2_Rvs_v1_008 | 4118 |
| PCR2_Rvs_v1_009 | 4119 |
| PCR2_Rvs_v1_010 | 4120 |
| PCR2_Rvs_v1_011 | 4121 |
| PCR2_Rvs_v1_012 | 4122 |
| PCR2_Rvs_v1_013 | 4123 |
| PCR2_Rvs_v1_014 | 4124 |
| PCR2_Rvs_v1_015 | 4125 |
| PCR2_Rvs_v1_016 | 4126 |
| PCR2_Rvs_v1_017 | 4127 |
| PCR2_Rvs_v1_018 | 4128 |
| PCR2_Rvs_v1_019 | 4129 |
| PCR2_Rvs_v1_020 | 4130 |
| PCR2_Rvs_v1_021 | 4131 |
| PCR2_Rvs_v1_022 | 4132 |
| PCR2_Rvs_v1_023 | 4133 |
| PCR2_Rvs_v1_024 | 4134 |
| PCR2_Rvs_v1_025 | 4135 |
| PCR2_Rvs_v1_026 | 4136 |
| PCR2_Rvs_v1_027 | 4137 |
| PCR2_Rvs_v1_028 | 4138 |
| PCR2_Rvs_v1_029 | 4139 |
| PCR2_Rvs_v1_030 | 4140 |
| PCR2_Rvs_v1_031 | 4141 |
| PCR2_Rvs_v1_032 | 4142 |
| PCR2_Rvs_v1_033 | 4143 |
| PCR2_Rvs_v1_034 | 4144 |
| PCR2_Rvs_v1_035 | 4145 |
| PCR2_Rvs_v1_036 | 4146 |
| PCR2_Rvs_v1_037 | 4147 |
| PCR2_Rvs_v1_038 | 4148 |
| PCR2_Rvs_v1_039 | 4149 |
| PCR2_Rvs_v1_040 | 4150 |
| PCR2_Rvs_v1_041 | 4151 |
| PCR2_Rvs_v1_042 | 4152 |
| PCR2_Rvs_v1_043 | 4153 |
| PCR2_Rvs_v1_044 | 4154 |
| PCR2_Rvs_v1_045 | 4155 |
| PCR2_Rvs_v1_046 | 4156 |
| PCR2_Rvs_v1_047 | 4157 |
| PCR2_Rvs_v1_048 | 4158 |
| PCR2_Rvs_v2_001 | 4159 |
| PCR2_Rvs_v2_002 | 4160 |
| PCR2_Rvs_v2_003 | 4161 |
| PCR2_Rvs_v2_004 | 4162 |
| PCR2_Rvs_v2_005 | 4163 |
| PCR2_Rvs_v2_006 | 4164 |
| PCR2_Rvs_v2_007 | 4165 |
| PCR2_Rvs_v2_008 | 4166 |
| PCR2_Rvs_v2_009 | 4167 |
| PCR2_Rvs_v2_010 | 4168 |
| PCR2_Rvs_v2_011 | 4169 |
| PCR2_Rvs_v2_012 | 4170 |
| PCR2_Rvs_v2_013 | 4171 |
| PCR2_Rvs_v2_014 | 4172 |
| PCR2_Rvs_v2_015 | 4173 |
| PCR2_Rvs_v2_016 | 4174 |
| PCR2_Rvs_v2_017 | 4175 |
| PCR2_Rvs_v2_018 | 4176 |
| PCR2_Rvs_v2_019 | 4177 |
| PCR2_Rvs_v2_020 | 4178 |
| PCR2_Rvs_v2_021 | 4179 |
| PCR2_Rvs_v2_022 | 4180 |
| PCR2_Rvs_v2_023 | 4181 |
| PCR2_Rvs_v2_024 | 4182 |
| PCR2_Rvs_v2_025 | 4183 |
| PCR2_Rvs_v2_026 | 4184 |
| PCR2_Rvs_v2_027 | 4185 |

### 3) Bacterial CRISPRi (CRISPR Interference) Assay

A dual-color fluorescence reporter screen was implemented, using monomeric Red Fluorescent Protein (mRFP) and Superfolder Green Fluorescent Protein (sfGFP), based on Qi L S, et al. (Cell 152, 5, 1173-1183 (2013)). This screen was utilized to assay gene-specific transcriptional repression mediated by programmable DNA binding of the CasX system). This strain of E. coli expresses bright green and red fluorescence under standard culturing conditions or when grown as colonies on agar plates. Under a CRISPRi system, the CasX protein is expressed from an anhydrotetracycline (aTc)-inducible promoter on a plasmid containing a p15A replication origin (plasmid pSTX3; chloramphenicol resistant), and the sgRNA is expressed from a minimal constitutive promoter on a plasmid containing a ColE1 replication origin (pSTX4, non-targeting spacer, or pSTX5, GFP-targeting spacer #1; carbenicillin resistant). When the E. coli strain is co-transformed with both plasmids, genes targeted by the spacer in pSTX4 are repressed; in this case GFP repression is observed, the degree to which is dependent on the function of the targeting CasX protein and sgRNA. In this system, RFP fluorescence can serve as a normalizing control. Specifically, RFP fluorescence should be unaltered and independent of functional CasX based CRISPRi activity. CRISPRi activity can be tuned in this system by regulating the expression of the CasX protein; here, all assays used an induction concentration of 20 nM aTc final concentration in growth media.

Libraries of sgRNA were constructed to assess the activity of sgRNA variants in complex with three cleavage-inactivating mutations made to the reference CasX protein open reading frame of Planctomycetes, SEQ ID NO: 2, rendering the CasX catalytically dead (dCasX). These three mutations are referred to as D1 (with a D659A substitution), D2 (with a E756A substitution), or D3 (with a D922A substitution). A fourth library, composed of all three mutations in combination is referred to as DDD (D659A; E756A; D922A substitutions).

Libraries of sgRNA were screened for activity using the above CRISPRi system with either D2, D3, or DDD. After co-transformation and recovery, libraries were grown for 8 hours in 2xyt media with appropriate antibiotics and sorted on a Sony MA900 flow cytometry instrument. Each library version was sorted with three different gates (in addition to the naive, unsorted library). Three different sort gates were employed to extract GFP—cells: 10%, 1%, and “F” which represents ˜0.1% of cells, ranked by GFP repression. Finally, each sort was done in two technical replicates. Variants of interest were detected using either Sanger sequencing of picked colonies (UC Berkeley Barker Sequencing Facility) or NGS sequencing of miniprepped plasmid (Massachusetts General Hospital CCIB DNA Core Next-Generation Sequencing Service) or NGS sequencing of PCR amplicons, produced with primers that introduced indexing adapters for sequencing on an Illumina platform (see section above). Amplicons were sent for sequencing with Novogene (Beijing, China) for sequencing on an Illumina Hiseq, with 150 cycle, paired-end reads. Each sorted sample had at least 3 million reads per technical replicate, and at least 25 million reads for the naive samples. The average read count across all samples was 10 million reads.

### 4) NGS Data Analysis

Paired end reads were trimmed for adapter sequences with cutadapt (version 2.1), merged to form a single read with flash2 (v2.2.00), and aligned to the reference with bowtie2 (v2.3.4.3). The reference was the entire amplicon sequence, which includes ˜30 base pairs flanking the Planctomyces reference guide scaffold from the plasmid backbone having the sequence:

(SEQ ID NO: 4221)
TGACAGCTAGCTCAGTCCTAGGTATAATACTAGTTACTGGCGCTTTTAT
CTCATTACTTTGAGAGCCATCACCAGCGACTATGTCGTATGGGTAAAGC
GCTTATTTATCGGAGAGAAATCCGATAAATAAGAAGCATCAAAGCTGGA
GTTGTCCCAATTCTTCTAGAG.

Variants between the reference and the read were determined from the bowtie2 output. In brief, custom software in python (analyzeDME/bin/bam_to_variants.py) extracted single-base variants from the reference sequence using the cigar string and and string from each alignment. Reads with poor alignment or high error rates were discarded (mapq <20 and estimated error rate >4%; estimated error rate was calculated using per-base phred quality scores). Single-base variants at locations of poor-quality sequencing were discarded (phred score <20). Immediately adjacent single-base variants were merged into one mutation that could span multiple bases. Mutations were labeled for being single substitutions, insertions, or deletions, or other higher-order mutations, or outside the scaffold sequence.

The number of reads that supported each set of mutations was determined. These read counts were normalized for sequencing depth (mean normalization), and read counts from technical replicates were averaged by taking the geometric mean.

To obtain enrichment values for each scaffold variant, the number of normalized reads for each sorted sample were compared to the average of the normalized read counts for D2 and D3, which were highly correlated (FIG. 41). The naive DDD sample was not sequenced. To obtain the enrichment for each catalytically dead CasX variant, the log of the enrichment values across the three sort gates were averaged.

Methods for Individual Validation of sgRNA Activity in Human Cell Assays
1) Individual sgRNA Variant Construction

In order to screen variants of interest, individual variants were constructed using standard molecular biology techniques. All mutations were built on the reference CasX (SEQ ID NO:2) using a staging vector and Gibson cloning. To build single mutations, a universal forward (5′→3′) and reverse (3′→5′) primer were designed on either end of the encoded protein sequence that had homology to the desired backbone for screening (see Table 37 below). Primers to create the desired mutations were also designed (F primer and its reverse complement) and used with the universal F and R primers for amplification; thus producing two fragments. In order to add multiple mutations, additional primers with overlap were designed and more PCR fragments were produced. For example, to construct a triple mutant, four sets of F/R primers were designed. The resulting PCR fragments were gel extracted. These fragments were subsequently assembled into a screening vector (see Table 37), by digesting the screening vector backbone with the appropriate restriction enzymes and gel extraction. The insert fragments and vector were then assembled using Gibson assembly master mix, transformed, and plated using appropriate LB agar+antibiotic. The clones were Sanger sequenced and correct clones were chosen.

Finally, spacer cloning was performed to target the guide RNA to a gene of interest in the appropriate assay or screen. The sequence-verified non-targeting clone was digested with the appropriate Golden Gate enzyme and cleaned using DNA Clean and Concentrator kit (Zymo). The oligos for the spacer of interest were annealed. The annealed spacer was ligated into a digested and cleaned vector using a standard Golden Gate Cloning protocol. The reaction was transformed into Turbo E. coli and plated on LB agar+carbenicillin, and allowed to grow overnight at 37° C. Individual colonies were picked the next day, grown for eight hours in 2XYT +carbenicillin at 37° C., and miniprepped. The clones were Sanger sequenced and correct clones were chosen.

TABLE 37
screening vectors and associated primer sequences

| Screening | | |
|---|---|---|
| vector | F primer sequence | R primer sequence |
| pSTX6 | SAH24: | SAH25: |
| | TTCAGGTTGGACCGGTGCCACCATGGCC | TTTTGGACTAGTCACGGCGGGC |
| | CCAAAGAAGAAGCGGAAGGTCAGCCAAG | TTCCAG (SEQ ID NO: |
| | AGATCAAGAGAATCAACAAGATCAGA | 4105) |
| | (SEQ ID NO: 4104) | |
| pSTX16 or | oIC539: | oIC540: |
| pSTX34 | ATGGCCCCAAAGAAGAAGCGGAAGGTCT | TACCTTTCTCTTCTTTTTTGGA |
| | CTAGACAAG (SEQ ID NO: 4106) | CTAGTCACGG (SEQ ID NO: |
| | | 4107) |

### 2) GFP Editing by Plasmid Lipofection of HEK293T Cells

Either doxycycline-inducible GFP (iGFP) reporter HEK293T cells or SOD1-GFP reporter HEK293T cells were seeded at 20-40 k cells/well in a 96 well plate in 100 μl of FB medium and cultured in a 37° C. incubator with 5% CO2. The following day, confluence of seeded cells was checked. Cells were ˜75% confluent at time of transfection. Each CasX construct was transfected at 100-500 ng per well using Lipofectamine 3000 following the manufacturer's protocol, into 3 wells per construct as replicates. SaCas9 and SpyCas9 targeting the appropriate gene were used as benchmarking controls. For each Cas protein type, a non-targeting plasmid was used as a negative control.

After 24-48 hours of puromycin selection at 0.3-3 μg/ml to select for successfully transfected cells, followed by 1-7 days of recovery in FB medium, GFP fluorescence in transfected cells was analyzed via flow cytometry. In this process, cells were gated for the appropriate forward and side scatter, selected for single cells and then gated for reporter expression (Attune Nxt Flow Cytometer, Thermo Fisher Scientific) to quantify the expression levels of fluorophores. At least 10,000 events were collected for each sample. The data were then used to calculate the percentage of edited cells.

### 3) GFP Editing by Lentivirus Transduction of HEK293T Cells

Lentivirus products of plasmids encoding CasX proteins, including controls, CasX variants, and/or CasX libraries, were generated in a Lenti-X 293T Cell Line (Takara) following standard molecular biology and tissue culture techniques. Either iGFP HEK293T cells or SOD1-GFP reporter HEK293T cells were transduced using lentivirus based on standard tissue culture techniques. Selection and fluorescence analysis was performed as described above, except the recovery time post-selection was 5-21 days. For Fluorescence-Activated Cell Sorting (FACS), cells were gated as described above on a MA900 instrument (Sony). Genomic DNA was extracted by QuickExtract™ DNA Extraction Solution (Lucigen) or Genomic DNA Clean & Concentrator (Zymo).

### Results:

Engineering of sgRNA 1 to 174
1) sgRNA Derived from Metagenomics of Bacterial Species Improved Function in Human Cells

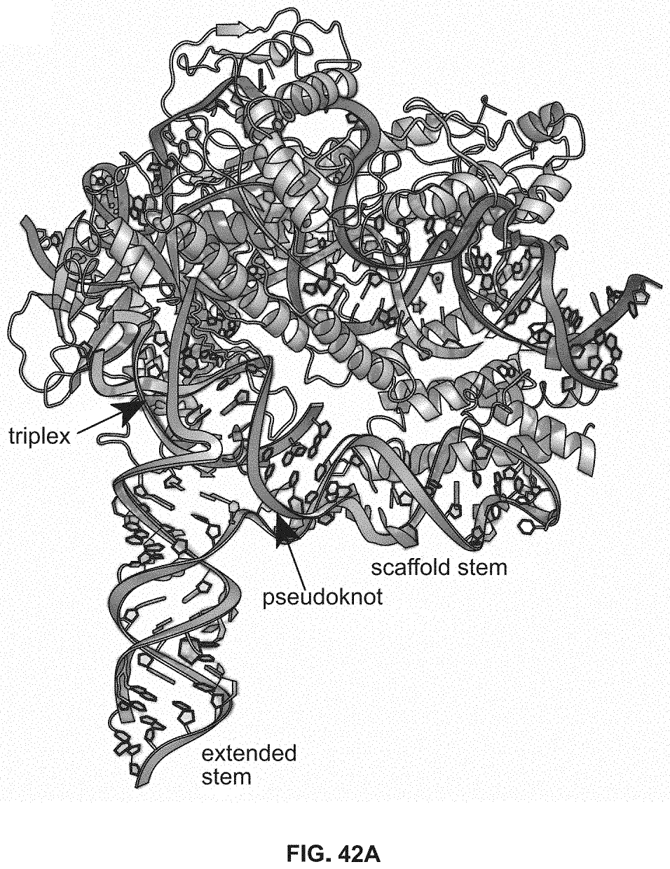

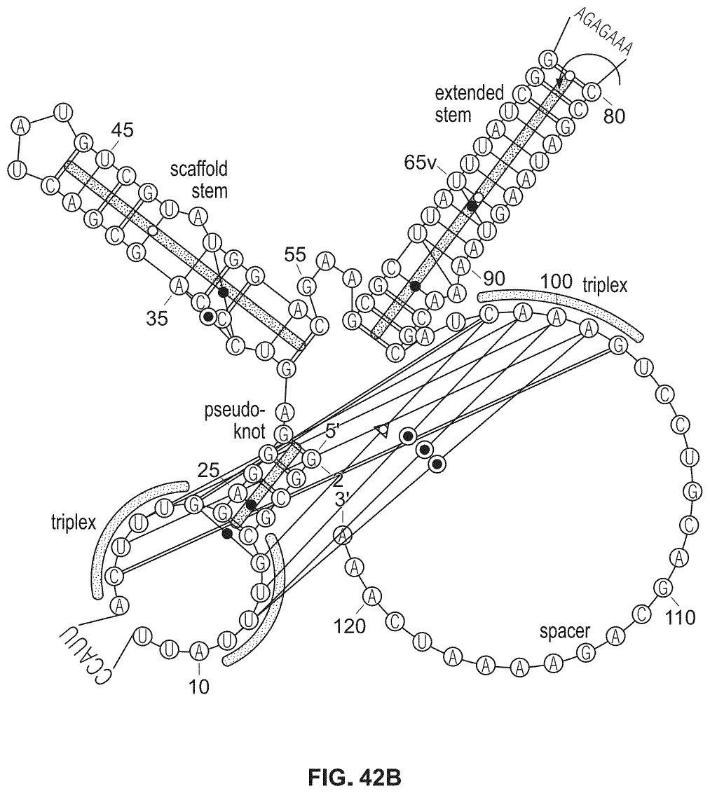

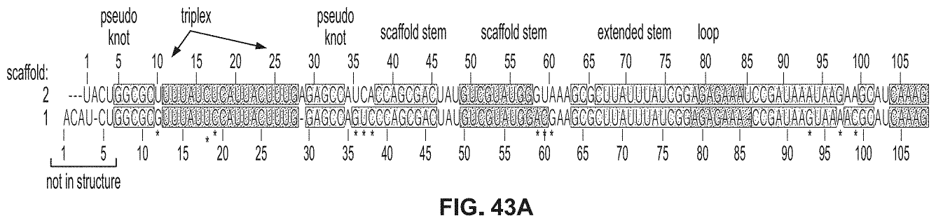

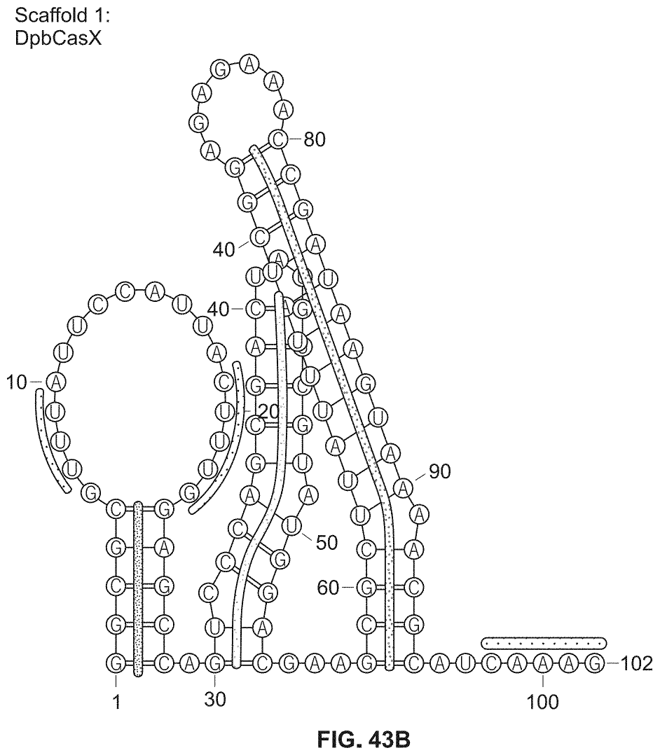

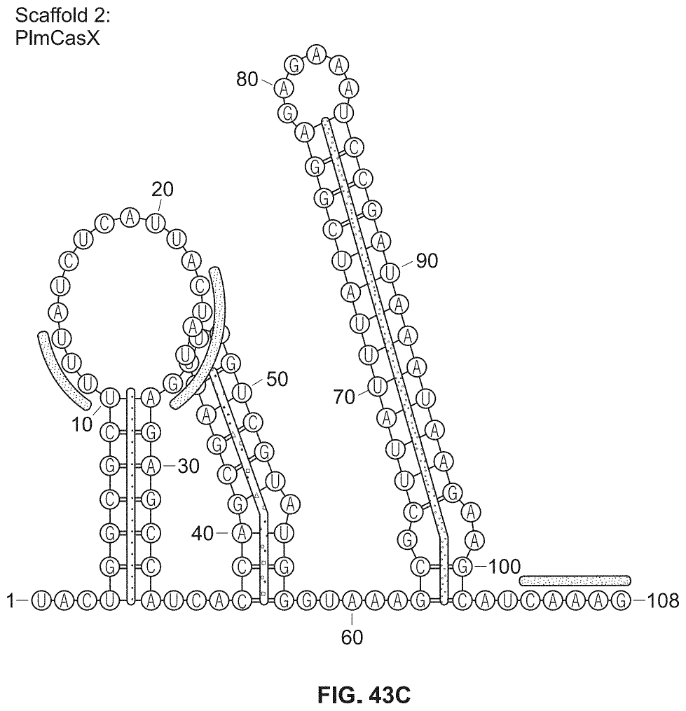

An initial improvement in CasX RNP cleavage activity was found by assessing new metagenomic bacterial sequences for possible CasX guide scaffolds. Prior work demonstrated that Deltaproteobacteria sgRNA (SEQ ID NO:4) could form a functional RNA-guided nuclease complex with CasX proteins, including the Deltaproteobacteria CasX (SEQ ID NO:1 or Planctomycetes CasX (SEQ ID NO:2). Structural characterization of this complex allowed identification of structural elements within the sgRNA (FIG. 42). However, a sgRNA scaffold from Planctomycetes was never tested. A second tracrRNA was identified from Planctomycetes, which was made into an sgRNA with the same method as was used for Deltaproteobacteria tracrRNA-crRNA (SEQ ID NO:5) (Liu, J J et al Nature, 566, 218-223 (2019)). These two sgRNA had similar structural elements, based on RNA secondary structure prediction algorithms, including three stem loop structures and possible triplex formation (FIG. 43).

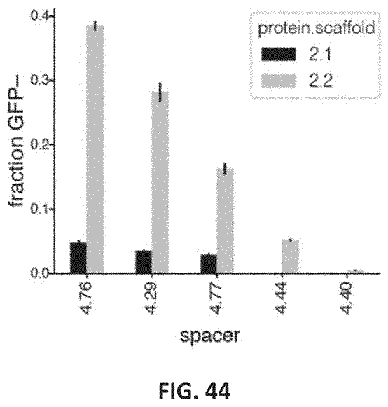

Characterization the activity of Planctomycetes CasX protein complexed with the Deltaproteobacteria sgRNA (hereafter called RNP 2.1, wherein the CasX protein has the sequence of SEQ ID NO:2) and Planctomycetes CasX protein complexed with scaffold 2 sgRNA (hereafter called RNP 2.2) showed clear superiority of RNP 2.2 compared to the others in a GFP-lipofection assay (see Methods) (FIG. 44). Thus, this scaffold formed the basis of our molecular engineering and optimization.

### 2) Improving Activity of CasX RNP Through Comprehensive RNA Scaffold Mutagenesis Screen.

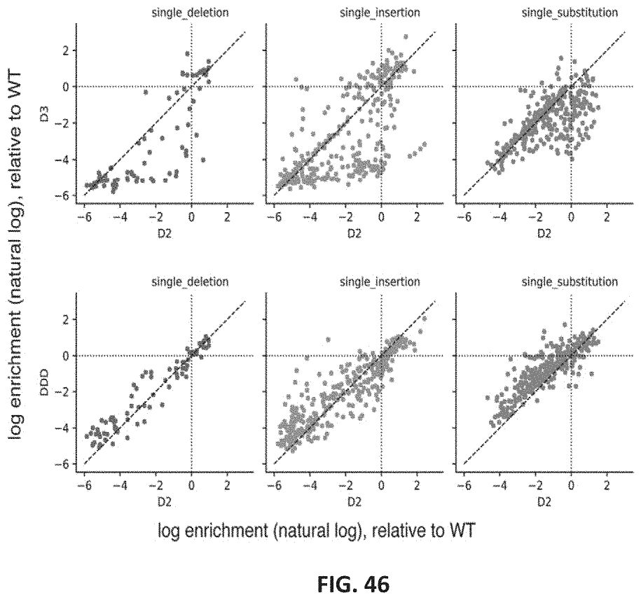

To find mutations to the guide RNA scaffold that could improve dsDNA cleavage activity of the CasX RNP, a large diversity of insertions, deletions and substitutions to the gRNA scaffold 2 were generated (see Methods). This diverse library was screened using CRISPRi to determine variants that improved DNA-binding capabilities and ultimately improved cleavage activity in human cells. The library was generated through a process of pooled primer cloning as described in the Materials and Methods. The CRISPRi screen was carried out using three enzymatically-inactive versions of CasX (called D2, D3, and DDD; see Methods). Library variants with improved DNA binding characteristics were identified through a high-throughput sorting and sequencing approach. Scaffold variants from cells with high GFP repression (i.e., low fluorescence) were isolated and identified with next generation sequencing. The representation of each variant in the GFP—pool was compared to its representation in the naive library to form an enrichment score per variant (see Materials and Methods). Enrichment was reproducible across the three catalytically dead-CasX variants (FIG. 46).

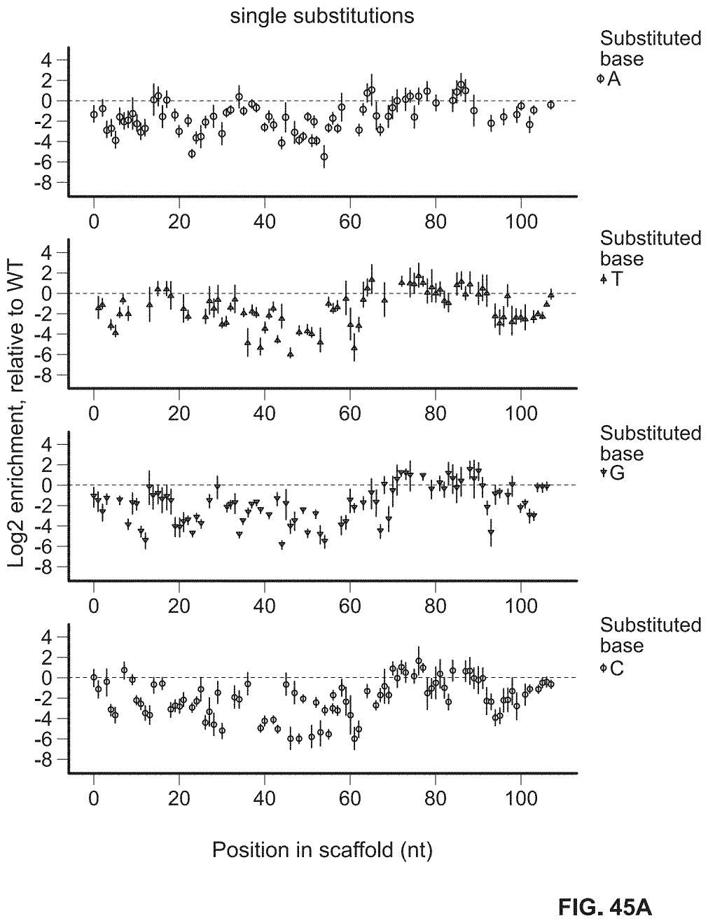

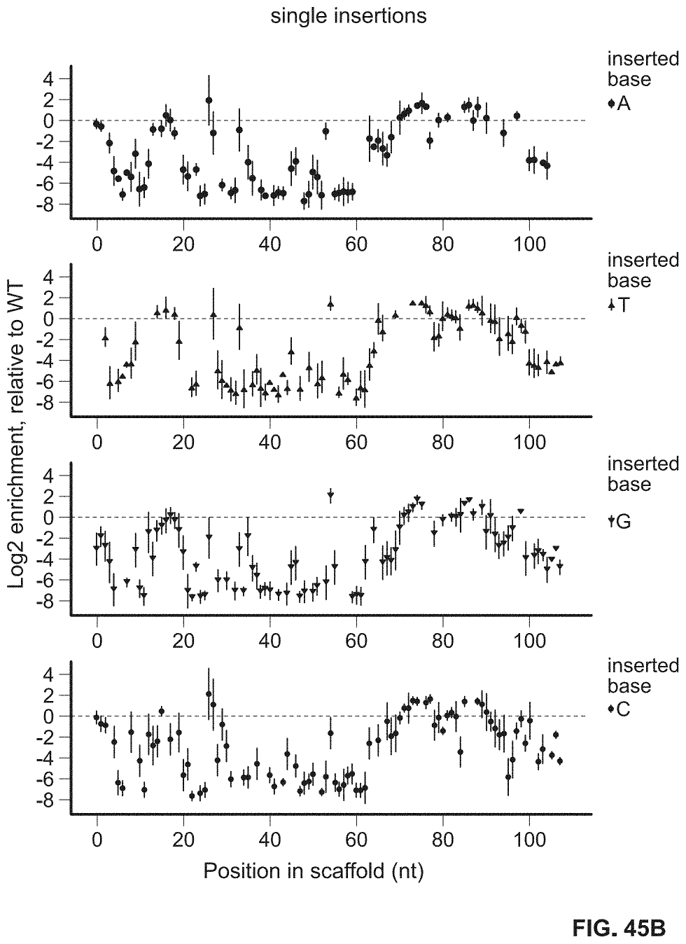

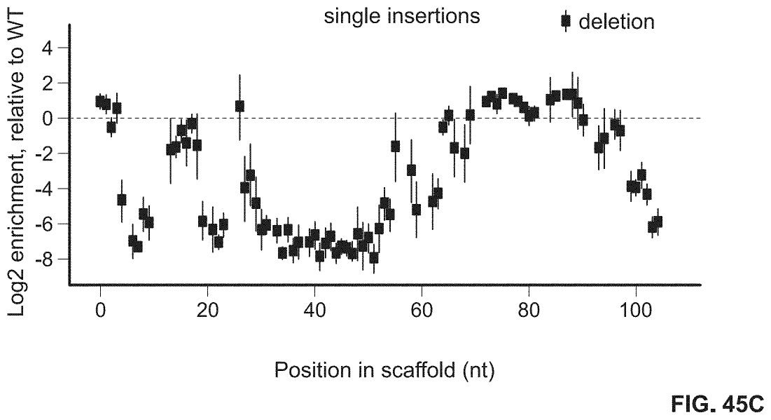

Examining the enrichment scores of all single variants revealed mutable locations within the guide scaffold, especially the extended stem (FIG. 45). The top-20 enriched single variants outside of the extended stem are listed in Table 38. In addition to the extended stem, these largely cluster into four regions: position 55 (scaffold stem bubble), positions 15-19 (triplex loop), position 27 (triplex), and in the 5′ end of the sequence (positions 1, 2, 4, 8). While the majority of these top-enriched variants were consistently enriched across all three catalytically dead CasX versions, the enrichment at position 27 was variable, with no evident enrichment in the D3 CasX (data not shown).

The enrichment of different structural classes of variants suggested that the RNP activity might be improved by distinct mechanisms. For example, specific mutations within the extended stem were enriched relative to the WT scaffold. Given that this region does not substantially contact the CasX protein (FIG. 42A), we hypothesize that mutating this region may improve the folding stability of the gRNA scaffold, while not affecting any specific protein-binding interaction interfaces. On the other hand, 5′ mutations could be associated with increased transcriptional efficiency. In a third mechanism, it was reasoned that mutations to the scaffold stem bubble or triplex could lead to increased stability through direct contacts with the CasX protein, or by affecting allosteric mechanisms with the RNP. These distinct mechanisms to improve RNP binding support that these mutations could be stacked or combined to additively improve activity.

TABLE 38
Top enriched single-variants outside of extended stem.

| | | | | log2 | |
|---|---|---|---|---|---|
| Position | Annotation | Reference | Alternate | enrichment | Region |
| 55 | insertion | — | G | 2.37466 | scaffold stem |
| | | | | | bubble |
| 55 | insertion | — | T | 1.93584 | scaffold stem |
| | | | | | bubble |
| 15 | insertion | — | T | 1.65155 | triplex loop |
| 17 | insertion | — | T | 1.56605 | triplex loop |
| 4 | deletion | T | — | 1.48676 | 5′ end |
| 27 | insertion | — | C | 1.26385 | triplex |
| 16 | insertion | — | C | 1.26025 | triplex loop |
| 19 | insertion | — | T | 1.25306 | triplex loop |
| 18 | insertion | — | G | 1.22628 | triplex loop |
| 2 | deletion | A | — | 1.17690 | 5′ end |
| 17 | insertion | — | A | 1.16081 | triplex loop |
| 18 | substitution | C | T | 1.10247 | triplex loop |
| 18 | insertion | — | A | 1.04716 | triplex loop |
| 16 | substitution | C | T | 0.97399 | triplex loop |
| 8 | substitution | G | C | 0.95127 | pseudoknot |
| 16 | substitution | C | A | 0.89373 | triplex loop |
| 27 | insertion | — | A | 0.86722 | triplex |
| 1 | substitution | T | C | 0.83183 | 5′ end |
| 18 | deletion | C | — | 0.77641 | triplex loop |
| 19 | insertion | — | G | 0.76838 | triplex loop |

3) Assessing RNA Scaffold Mutants in dsDNA Cleavage Assay in Human Cells

The CRISPRi screen is capable of assessing binding capacity in bacterial cells at high throughput; however it does not guarantee higher cleavage activity in human cell assays. We next assessed a large swath of individual scaffold variants for cleavage capacity in human cells using a plasmid lipofection in HEK cells (see Materials and Methods). In this assay, human HEK293T cells containing a stably-integrated GFP gene are transduced with a plasmid (p16) that expresses reference CasX protein (Stx2) (SEQ ID NO: 2) and sgRNA comprising the gRNA scaffold variant and spacers 4.76 (having sequence UGUGGUCGGGGUAGCGGCUG (SEQ ID NO: 4222) and 4.77 (having sequence UCAAGUCCGCCAUGCCCGAA (SEQ ID NO: 4223)) to target the RNP to knockdown the GFP gene. Percent GFP knockdown was assayed using flow cytometry. Over a hundred scaffold variants were tested in this assay.

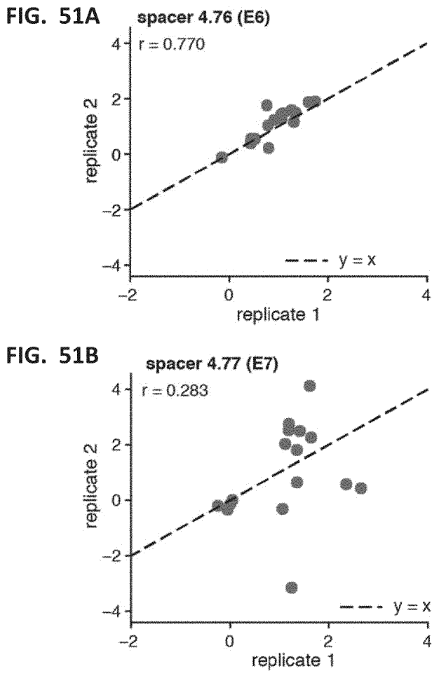

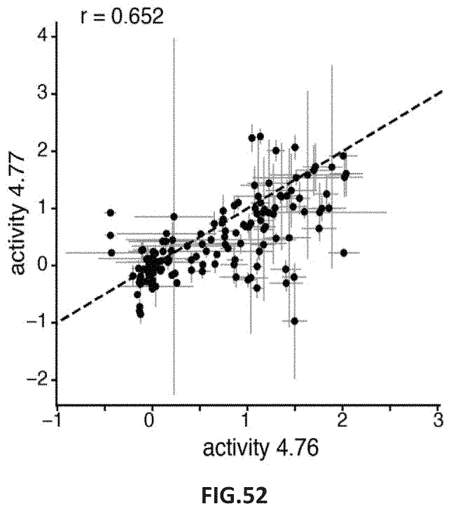

The assay resulted in largely reproducible values across different assay dates for spacer 4.76, while exhibiting more variability for spacer 4.77 (FIG. 51). Spacer 4.77 was generally less active for the wild-type RNP complex, and the lower overall signal may have contributed to this increased variability. Comparing the cleavage activity across the two spacers showed generally correlated results (r=0.652; FIG. 52). Because of the increased noise in spacer 4.77 measurements, the reported cleavage activity per scaffold was taken as the weighted average between the measurements on each scaffold, with the weights equal to the inverse squared error. This weighting effectively down-weights the contribution from high-error measurements.

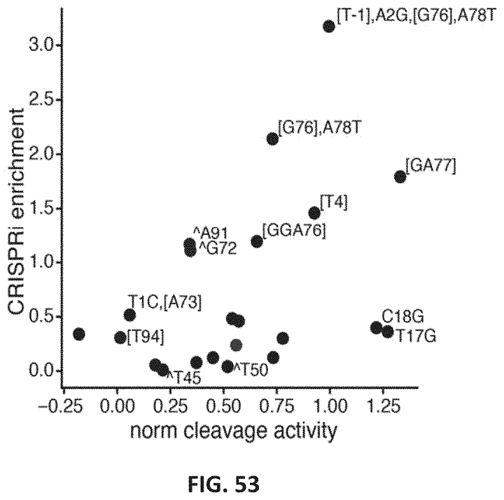

A subset of sequences was tested in both the HEK-iGFP assay and the CRISPRi assay. Comparing the CRISPRi enrichment score to the GFP cleavage activity showed that highly-enriched variants had cleavage activity at or exceeding the wildtype RNP (FIG. 45C). Two variants had high cleavage activity with low enrichment scores (C18G and T17G); interestingly, these substitutions are at the same position as several highly-enriched insertions (FIG. 53).

Examining all scaffolds tested in the HEK-iGFP assay revealed certain features that consistently improved cleavage activity. We found that the extended stem could often be completely swapped out for a different stem, with either improved or equivalent activity (e.g., compare scaffolds of SEQ ID NO: 2101-2105, 2111, 2113, 2115; all of which have replaced the extended stem, with increased activity relative to the reference, as seen in Table 27). We specifically focused on two stems with different origins: a truncated version of the wildtype stem, with the loop sequence replaced by the highly stable UUCG tetraloop (stem 42). The other (stem 46) was derived from Uvsx bacteriophage T4 mRNA, which in its biological context is important for regulation of reverse transcription of the bacteriophage genome (Tuerk et al. Proc Natl Acad Sci USA. 85(5):1364 (1988)). The top-performing gRNA scaffolds all had one of these two extended stem versions (e.g., SEQ ID NOS: 2160 and 2161).

Appending ribozymes to the 3′ end often resulted in functional scaffolds (e.g., see SEQ ID NO: 2182 with equivalent activity to the WT guide in this assay {Table 27}). On the other hand, adding to the 5′ end generally hurt cleavage activity. The best-performing 5′ ribozyme construct (SEQ ID NO:2208) had cleavage activity <40% of the WT guide in the assay.

Certain single-point mutations were generally good, or at least not harmful, including T 10C, which was designed to increase transcriptional efficiency in human cells by removing the four consecutive T's at the 5′ start of the scaffold (Kiyama and Oishi. Nucleic Acids Res., 24:4577 (1996)). C18G was another helpful mutation, which was obtained from individual colony picking from the CRISPRi screen. The insertion of C at position 27 was highly-enriched in two out of the three dCasX versions of the CRISPRi screen; however, it did not appear to help cleavage activity. Finally, insertion at position 55 within the RNA bubble substantially improved cleavage activity (i.e., compare SEQ ID NO: 2236, with a \hat{ }G55 insertion to SEQ ID NO:2106 in Table 27).

### 4) Further Stacking of Variants in Higher-Stringency Cleavage Assays

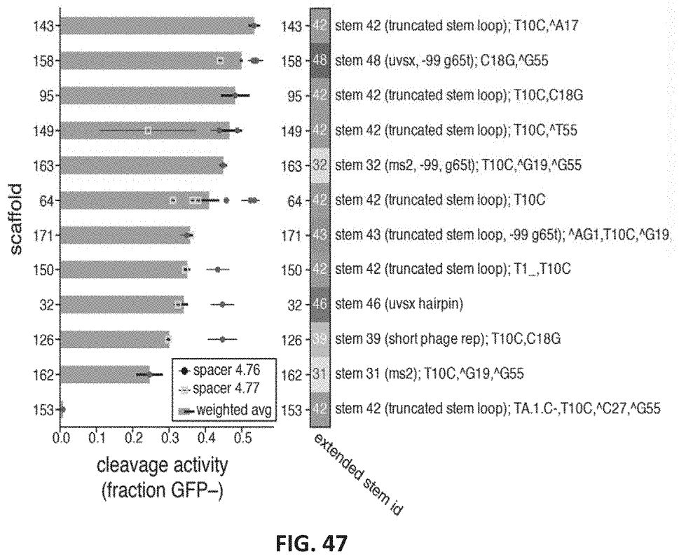

Scaffold mutations that proved beneficial were stacked together to form a set of new variants that were tested under more stringent criteria: a plasmid lipofection assay in human HEK-293t cells with the GFP gene knocked into the SOD1 allele, which we observed was generally harder to knock down. Of this batch of variants, guide scaffold 158 was identified as a top-performer (FIG. 47). This scaffold had a modified extended stem (Uvsx), with additional mutations to fully base pair the extended stem ([A99] and G65U). It also contained mutations in the triplex loop (C18G) and in the scaffold stem bubble (\hat{ }G55).

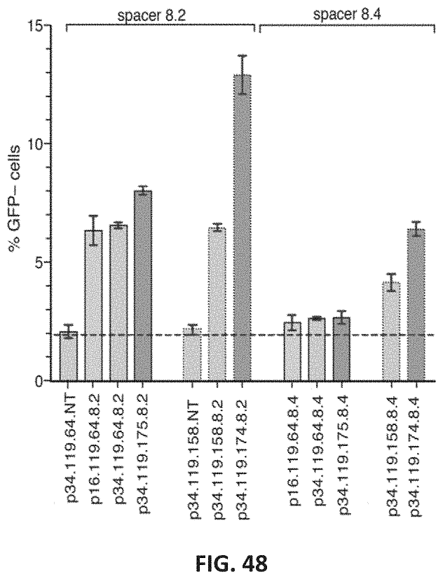

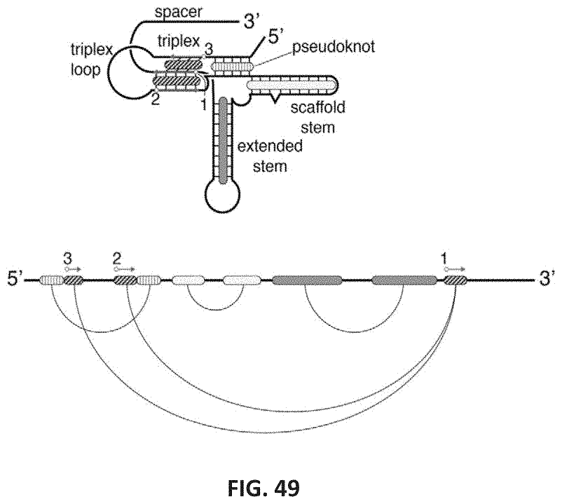

In a second validation of improved DNA editing capacity, sgRNAs were delivered to cells with low-MOI lentiviral transduction, and with distinct targeting sequences to the SOD1 gene (see Methods); spacers were 8.2 (having sequence AUGUUCAUGAGUUUGGAGAU (SEQ ID NO: 4224)), and 8.4 (having sequence UCGCCAUAACUCGCUAGGCC (SEQ ID NO: 4225)) (results shown in FIG. 48). Additionally, 5′ truncations of the initial GT of guide scaffolds 158 and 64 were deleted (forming scaffolds 174 and 175 respectively). This assay showed dominance of guide scaffold 174: the variant derived from guide scaffold 158 with 2 bases truncated from the 5′ end (FIG. 48). A schematic of the secondary structure of scaffold 174 is shown in FIG. 49.

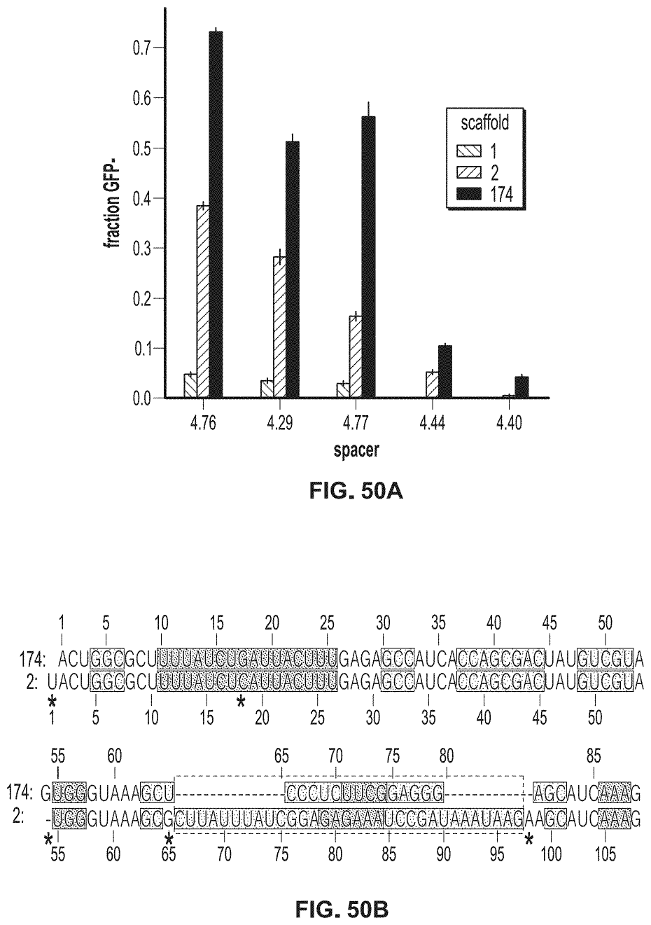

In sum, our improved guide scaffold 174 showed marked improvement over our starting reference guide scaffold (scaffold 1 from Deltaproteobacteria, SEQ ID NO:4), and substantial improvement over scaffold 2 (SEQ ID NO:5) (FIG. 50). This scaffold contained a swapped extended stem (replacing 32 bases with 14 bases), additional mutations in the extended stem ([A99] and G65U), a mutation in the triplex loop (C18G), and in the scaffold stem bubble (AG55) (where all the numbering refers to the scaffold 2). Finally, the initial T was deleted from scaffold 2, as well as the G that had been added to the 5′ end in order to enhance transcriptional efficiency. The substantial improvements seen with guide scaffold 174 came collectively from the indicated mutations.

### Example 18: Design of Improved Guides Based on Predicted Secondary Structure Stability Methods

A computational method was employed to predict the relative stability of the ‘target’ secondary structure, compared to alternative, non-functional secondary structures. First, the ‘target’ secondary structure of the gRNA was determined by extracting base-pairs formed within the RNA in the CryoEM structure for CasX 1.1. For prediction of RNA secondary structure, the program RNAfold was used (version 2.4.14). The ‘target’ secondary structure was converted to a ‘constraint string’ that enforces bases to be paired with other bases, or to be unpaired. Because the triplex is unable to be modeled in RNAfold, the bases involved in the triplex are required to be unpaired in the constraint string, whereas all bases within other stems (pseudoknot, scaffold, and extended stems) were required to be appropriately paired. For guide scaffolds 2 (SEQ ID NO:5), 174 (SEQ ID NO:2238), and 175 (SEQ ID NO:2239), this constraint string was constructed based on sequence alignment between the scaffold and scaffold 1 (SEQ ID NO: 4) outside of the extended stem, which can have minimal sequence identity. Within the extended stem, bases were assumed to be paired according to the predicted secondary structure for the isolated extended stem sequence. See Table 39 for a subset of sequences and their constraint strings.

TABLE 39
Constraint strings to represent the ‘target secondary structure’ in RNAfold algorithm.

| Name | Constraint string |
|---|---|
| Scaffold 1 (w/5′ | (((((.xxx.........xxxxx))))).((.((((((((...))))).)))))...(((((((((((((((. |
| truncation as in | ......))))))))))).))))..xxxxx |
| CryoEM structure) | |
| Scaffold 2 | ....(((((.xxx.........xxxxx.)))))....((((((((...))))).))).....((.(((((((((( |
| | (((......)))))))))))))..))..xxxxx |
| Scaffold 174 | ...(((((.xxx.........xxxxx.)))))....((((((((...)))))..))).....((((((((....)) |
| | ))))))..xxxxx |
| Scaffold 175 | ...(((((.xxx.........xxxxx.)))))....((((((((...))))).))).....((.(((((((((... |
| | .)))))))))..))..xxxxx |

Secondary structure stability of the ensemble of structures that satisfy the constraint was obtained, using the command: ‘RNAfold-p0--noPS-C’ And taking the ‘free energy of ensemble’ in kcal/mol (ΔG_constraint). The prediction was repeated without the constraint to get the secondary structure stability of the entire ensemble that includes both the target and alternative structures, using the command: ‘RNAfold-p0--noPS’ and taking the ‘free energy of ensemble’ in kcal/mol (ΔG_all).

The relative stability of the target structure to alternate structures was quantified as the difference between these two ΔG values: ΔΔG=ΔG_constraint−ΔG_all. A sequence with a large value for LAG is predicted to have many competing alternate secondary structures that would make it difficult for the RNA to fold into the target binding-competent structure. A sequence with a low value for ΔΔG is predicted to be more optimal in terms of its ability to fold into a binding-competent secondary structure.

### Results

A series of new scaffolds was designed to improve scaffold activity based on existing data and new hypotheses. Each new scaffold comprised a set of mutations that, in combination, were predicted to enable higher activity of dsDNA cleavage. These mutations fell into the following categories: First, mutations in the 5′ unstructured region of the scaffold were predicted to increase transcription efficiency or otherwise improve activity of the scaffold. Most commonly, scaffolds had the 5′ “GU” nucleotides deleted (scaffolds 181-220: SEQ ID NOS: 2242-2280). The “U” is the first nucleotide (U1) in the reference sequence SEQ ID NO:5. The G was prepended to increase transcription efficiency by U6 polymerase. However, removal of these two nucleotides was shown, surprisingly, to increase activity (FIG. 66). Additional mutations at the 5′ end include (a) combining the GU deletion with A2G, such that the first transcribed base is the G at position 2 in the reference scaffold (scaffold 199: SEQ ID NO:2259); (b) deleting only U1 and keeping the prepended G (scaffold 200: SEQ ID NO:2260); and (c) deleting the U at position 4, which is predicted to be unstructured and was found to be beneficial when added to scaffold 2 in a high-throughput CRISPRi assay (scaffold 208: SEQ ID NO:2268).

A second class of mutations was to the extended stem region. The sequence for this region was chosen from three possible options: (a) a “truncated stem loop” which has a shorter loop sequence than the reference sequence extended stem (the scaffolds 64 and 175 contain this extended stem: SEQ ID NOS: 2106 and 2239, respectively) (b) Uvsx hairpin with additional loop-distal mutations [A99] and G65U to fully base-pair the extended stem (the scaffold 174: SEQ ID NO: 2238) contains this extended stem); or (c) an “MS2(U15C)” hairpin with the same additional loop-distal mutations [A99] and G65U as in (b). These three extended stems classes were present in scaffolds with high activity (e.g. see FIG. 65), and their sequences can be found in Table 40.

TABLE 40
Sequences of extended stem regions used in novel scaffolds.

| | | Incorporated in Scaffolds |
|---|---|---|
| Extended stem name | Extended stem sequence | (SEQ ID NO) |
| truncated stem | GCGCUUACGGACUUCGGUCCGUAAG | 2239, 2242-2244, 2246, |
| loop | AAGC (SEQ ID NO: 4226) | 2255-2258 |
| UvsX, -99 G65U | GCUCCCUCUUCGGAGGGAGC (SEQ | 2238, 2245, 2250-2254, |
| | ID NO: 4227) | 2259-2280 |
| MS2(U15C), -99 | GCUCACAUGAGGAUCACCCAUGUGA | 2249 |
| G65U | GC (SEQ ID NO: 4228) | |

Thirdly, a set of mutations was designed to the triplex loop region. This region was not resolved in the CryoEM structure of CasX 1.1, likely because it does not form base-pairs and thus is more flexible. This region tolerates mutations, with certain mutations having beneficial effects on RNP binding, based on CRISPRi data from scaffold 2 (FIG. 63). The C18G substitution within the triplex loop was already incorporated in the scaffold 174. The following mutations were added to scaffold 174, that were not immediately adjacent to the C18G substitution in order to limit potential negative epistasis between these mutations: \hat{ }U15 (insertion of U before nucleotide 15 in scaffold 2), \hat{ }U17, and C16A (scaffolds 208, 210, and 209: SEQ ID NOS: 2268, 2270, 2269, respectively).

Fourth, a set of mutations was designed to systematically stabilize the target secondary structure for the scaffold. For background, RNA polymers fold into complex three-dimensional structures that enforce their function. In the CasX RNP, the RNA scaffold forms a structure comprising secondary structure elements such as the pseudoknot stem, a triplex, a scaffold stem-loop, and an extended stem-loop, as evident in the Cryo-EM characterization of the CasX RNP 1.1. These structural elements likely help enforce a three dimensional structure that is competent to bind the CasX protein, and in turn enable conformational transitions necessary for enzymatic function of the RNP. However, an RNA sequence can fold into alternate secondary structures that compete with the formation of the target secondary structure. The propensity of a given sequence to fold into the target versus alternate secondary structures was quantified using computational prediction, similar to the method described in (Jarmoskaite, I., et al. 2019. A quantitative and predictive model for RNA binding by human pumilio proteins. Molecular Cell 74(5), pp. 966-981.e18.) for correcting observed binding equilibrium constants for a distinct protein-RNA interaction, and using RNAfold (Lorenz, R., Bernhart, S. H., Honer Zu Siederdissen, C., et al. 2011. ViennaRNA Package 2.0. Algorithms for Molecular Biology 6, p. 26) to predict secondary structure stability (see Methods).

A series of mutations were chosen that were predicted to help stabilize the target secondary structure, in the following regions: The pseudoknot is a base-paired stem that forms between the 5′ sequence of the scaffold and sequence 3′ of the triplex and triplex loop. This stem is predicted to comprise 5 base-pairs, 4 of which are canonical Watson-Crick pairs and the fifth is a noncanonical G:A wobble pair. Converting this G:A wobble to a Watson Crick pair is predicted to stabilize alternative secondary structures relative to the target secondary structure (high ΔΔG between target and alternative secondary structure stabilities; Methods). This aberrant stability comes from a set of secondary structures in which the triplex bases are aberrantly paired. However, converting the G to an A or a C (for an A:A wobble or C:A wobble) was predicted to lower the ΔΔG value (G8C or G8A added to scaffolds 174 and 175+C18G). A second set of mutations was in the triplex loop: including a U15C mutation and a C18G mutation (for scaffold 175 that does not already contain this variant). Finally, the linker between the pseudoknot stem and the scaffold stem was mutated at position 35 (U35A), which was again predicted to stabilize the target secondary structure relative to alternatives.

Scaffolds 189-198 (SEQ ID NOS:2250-2258) included these predicted mutations on top of scaffolds 174 or 175, individually and in combination. The predicted change in ΔΔG for each of these scaffolds is given in Table 41 below. This algorithm predicts a much stronger effect on ΔΔG with combining multiple of these mutations into a single scaffold.

TABLE 41
Predicted effect on target secondary structure stability of incorporating
specific mutations individually or in combination to scaffolds 174 or 175.

| | | | Effect of |
|---|---|---|---|
| | | | mutations(s) ΔΔG_mut- |
| Starting | | Scaffold ΔΔG | ΔΔG_starting_scaffold |
| scaffold | Mutation(s) | (kcal/mol) | (kcal/mol) |
| 174 | — | 0.17 | — |
| 174 | G8A | −0.74 | −0.91 |
| 174 | G8C | −0.32 | −0.49 |
| 174 | U15C | −0.02 | −0.19 |
| 174 | U35A | −0.22 | −0.39 |
| 174 | G8A, U15C, | −1.34 | −1.51 |
| | U35A | | |
| 175 | — | 3.23 | — |
| 175 | G8A | 3.15 | −0.08 |
| 175 | G8C | 3.15 | −0.08 |
| 175 | U35A | 3.07 | −0.16 |
| 175 | U15C | 0.78 | −2.45 |
| 175 | C18G | 0.43 | −2.80 |
| 175 | G8A, T15C, | −1.03 | −4.26 |
| | C18G, T35A | | |

A fifth set of mutations was designed to test whether the triplex bases could be replaced by an alternate set of three nucleotides that are still able to form triplex pairs (Scaffolds 212-220: SEQ ID NOS:2272-2280). A subset of these substitutions are predicted to prevent formation of alternate secondary structures.

A sixth set of mutations were designed to change the pseudoknot-triplex boundary nucleotides, which are predicted to have competing effects on transcription efficiency and triplex formation. These include scaffolds 201-206 (SEQ ID NOS:2261-2266).

1. A method of selecting an improved biomolecule variant, wherein the biomolecule variant is a protein, RNA, or DNA, comprising:
(i) constructing a library comprising a plurality of biomolecule variants;
wherein each variant is independently a variant of the same reference biomolecule, wherein each variant comprises an alteration of one or more monomer locations of the reference biomolecule, wherein the monomer is an amino acid of the protein or a ribonucleotide of the RNA or a deoxyribonucleotide of the DNA,
wherein each alteration of a monomer location is independently selected from the group consisting of substitution of the monomer, deletion of one or more consecutive monomers beginning at the location, and insertion of one or more consecutive monomers adjacent to the location; and
wherein the library represents variants comprising alteration of one or more locations for at least 1% of the monomer locations of the reference biomolecule;
(ii) screening the library of (i);
(iii) identifying at least a portion of the library of (i) that exhibits one or more improved characteristics compared to the reference biomolecule; and
(iv) selecting the improved biomolecule variant from the at least a portion of the library, wherein the improved biomolecule variant exhibits one or more improved characteristics compared to the reference biomolecule.

2. The method of claim 1, further comprising screening the portion of the library identified in step (iii).

3-4. (canceled)

5. A method of selecting an improved biomolecule variant, wherein the biomolecule is a protein, RNA, or DNA, comprising:
(i) constructing a library comprising a plurality of biomolecule variants;
wherein each variant is independently a variant of the same reference biomolecule, wherein each variant comprises an alteration of one or more monomer locations of the reference biomolecule, wherein the monomer is an amino acid of the protein or ribonucleotide of the RNA or deoxyribonucleotide of the DNA,
wherein each alteration of a monomer location is independently selected from the group consisting of substitution of the monomer, deletion of one or more consecutive monomers beginning at the location, and insertion of one or more consecutive monomers adjacent to the location; and
wherein the library represents variants comprising alteration of one or more locations of at least 5%, at least 10%, at least 30%, at least 70%, or at least 90% of the monomer locations of the reference biomolecule;
(ii) screening the library of (i);
(iii) identifying at least a portion of the library of (i) that exhibits one or more improved characteristics compared to the reference biomolecule;
(iv) carrying out one or more additional rounds of library construction and screening to produce a final library, wherein construction of each library comprises:
altering one or more additional monomer locations of the identified portion of the previous library to produce a subsequent library of biomolecule variants;
(v) selecting the improved biomolecule variant from the final library of biomolecule variants, wherein the improved biomolecule variant exhibits one or more improved characteristics compared to the reference biomolecule.

6. The method of claim 1, wherein the library in step (i) comprises biomolecule variants with a single alteration of a single monomer location, biomolecule variants with a single alteration of two monomer locations, and biomolecule variants with a single alteration of three monomer locations, wherein each alteration is independently selected from the group consisting of substitution of the monomer, deletion of one or more consecutive monomers beginning at the location, and insertion of one or more consecutive monomers adjacent to the location.

7-16. (canceled)

17. The method of claim 1, wherein the reference biomolecule is a CRISPR associated protein selected from the group consisting of CasX, CasY, Cas9, Cas12a, Cas12b, Cas12c, Cas12f, Cas12g, Cas12h, Cas12i, Cas12j, Cas13a, Cas13b, Cas13c, Cas13d, Cas14, CASCADE, CSM, and CSY.

18.-19. (canceled)

20. The method of claim 17, wherein the one or more improved characteristics are independently selected from the group consisting of improved folding of the variant, improved binding affinity to the guide RNA, improved binding affinity to a target DNA, altered binding affinity to one or more PAM sequences, improved unwinding of a target DNA, increased activity, improved editing efficiency, improved editing specificity, increased activity of the nuclease, increased target strand loading for double strand cleavage, decreased target strand loading for single strand nicking, decreased off-target cleavage, decreased off-target binding/nicking, improved binding of the non-target strand of a DNA, improved protein stability, improved protein:guide-RNA complex stability, improved protein solubility, improved protein:guide NA complex stability, improved protein yield, increased collateral activity, and decreased collateral activity.

21. (canceled)

22. The method of claim 1, wherein the reference biomolecule is a CRISPR guide RNA that binds to CasX, CasY, Cas9, Cas12a, Cas12b, Cas12c, Cas12f, Cas12g, Cas12h, Cas12i, Cas12j, Cas13a, Cas13b, Cas13c, Cas13d, Cas14, CASCADE, CSM, or CSY.

23. (canceled)

24. The method of claim 22, wherein the one or more improved characteristics are independently selected from the group consisting of improved stability, improved solubility, improved resistance to nuclease activity, improved binding affinity to a reference CRISPR associated protein, improved binding affinity to a target DNA, improved gene editing, and improved specificity.

25-30. (canceled)

31. A method of constructing a library of polynucleotide variants of a reference biomolecule, comprising:
(a) constructing a polynucleotide that encodes for a variant of the reference biomolecule, wherein the reference biomolecule is a protein or RNA or DNA;
wherein the polynucleotide encodes for an alteration of one or more monomer locations of the reference biomolecule, wherein the monomer is an amino acid of the protein or ribonucleotide of the RNA or the deoxyribonucleotide of the DNA, and
wherein each alteration of a monomer location is independently selected from the group consisting of substitution of the monomer, deletion of one or more consecutive monomers beginning at the location, and insertion of one or more consecutive monomers adjacent to the location; and
(b) repeating the polynucleotide construction of (a) a sufficient number of times such that the library of polynucleotide represents variants comprising a single alteration of a single location for at least of at least 5%, at least 10%, at least 30%, at least 70%, or at least 90%1% of the monomer locations of the biomolecule.

32-42. (canceled)

43. The method of claim 31, wherein the reference biomolecule is a protein, and wherein substitution of the monomer comprises replacing the monomer with one of the nineteen other naturally occurring amino acids.

44-46. (canceled)

47. The method of claim 31, wherein the reference biomolecule is an RNA, and wherein substitution of the monomer comprises replacing the monomer with one of the three other naturally occurring ribonucleotides.

48-53. (canceled)

54. The method of claim 31 wherein the reference biomolecule is a CRISPR associated protein selected from the group consisting of CasX, CasY, Cas9, Cas12a, Cas12b, Cas12c, Cas12f, Cas12g, Cas12h, Cas12i, Cas12j, Cas13a, Cas13b, Cas13c, Cas13d, Cas14, CASCADE, CSM, and CSY.

55-60. (canceled)

61. The method of claim 31 wherein the reference biomolecule is a CRISPR guide RNA wherein the CRISPR guide RNA is a guide RNA that binds to CasX, CasY, Cas9, Cas12a, Cas12b, Cas12c, Cas12f, Cas12g, Cas12h, Cas12i, Cas12j, Cas13a, Cas13b, Cas13c, Cas13d, Cas14, CASCADE, CSM, or CSY.

62-64. (canceled)

65. A polynucleotide variant library, comprising polynucleotide variants of a reference biomolecule, comprising:
a plurality of polynucleotides that independently encode for a variant of the reference biomolecule, wherein the reference biomolecule is a protein or RNA or DNA;
wherein each polynucleotide independently encodes an alteration of one or more monomer locations of the reference biomolecule, wherein the monomer is an amino acid of the protein or ribonucleotide of the RNA or deoxyribonucleotide of the DNA, and
wherein each alteration of a monomer location is independently selected from the group consisting of substitution of the monomer, deletion of one or more consecutive monomers beginning at the location, and insertion of one or more consecutive monomers adjacent to the location; and
wherein the library of polynucleotides represents variants comprising a single alteration of a single location of at least 5%, at least 10%, at least 30%, at least 70%, or at least 90% for at least 1% of the monomer locations.

66-76. (canceled)

77. The polynucleotide variant library of claim 65, wherein the reference biomolecule is a protein, and wherein substitution of the monomer comprises replacing the monomer with one of the nineteen other naturally occurring amino acids.

78-81. (canceled)

82. The polynucleotide variant library of claim 65, wherein the reference biomolecule is an RNA, and wherein substitution of the monomer comprises replacing the monomer with one of the three other naturally occurring ribonucleotides.

83-86. (canceled)

87. The polynucleotide variant library of claim 65, wherein the reference biomolecule is a CRISPR associated protein, and wherein the CRISPR associated protein is CasX, CasY, Cas9, Cas12a, Cas12b, Cas12c, Cas12f, Cas12g, Cas12h, Cas12i, Cas12j, Cas13a, Cas13b, Cas13c, Cas13d, Cas14, CASCADE, CSM, or CSY.

88-93. (canceled)

94. The polynucleotide variant library of claim 65, wherein the reference biomolecule is a CRISPR guide RNA, and wherein the CRISPR guide RNA is a guide RNA that binds to CasX, CasY, Cas9, Cas12a, Cas12b, Cas12c, Cas12f, Cas12g, Cas12h, Cas12i, Cas12j, Cas13a, Cas13b, Cas13c, Cas13d, Cas14, CASCADE, CSM, or CSY.

95-110. (canceled)

111. A library of variant oligonucleotides, wherein:
each variant oligonucleotide independently encodes an alteration of one or more sequential monomer locations of a reference biomolecule, wherein:
the reference biomolecule is a protein, RNA, or DNA,
the one or more monomers are one or more amino acids of the protein or ribonucleotides of the RNA or one or more deoxyribonucleotides of DNA, and
wherein each alteration of a monomer location is independently selected from the group consisting of substitution of the monomer, deletion of one or more consecutive monomers beginning at the location, and insertion of one or more consecutive monomers adjacent to the location;
each variant oligonucleotide comprises a pair of homology arms flanking the encoded alteration, wherein the homology arms are homologous to the reference biomolecule sequences flanking the corresponding monomer location alteration, and wherein each homology arm independently comprises between 10 to 100 nucleotides; and
the library of variant oligonucleotides represents alteration of a single monomer for at least 80% of monomer locations.

112. The library of variant oligonucleotides of claim 111, wherein each variant oligonucleotide independently encodes an alteration of one or more monomer locations of the reference biomolecule.

113. A library comprising a plurality of RNA variants, wherein each variant is independently a variant of the same reference RNA, and each variant comprises a point mutation, deletion, or insertion at one ribonucleotide location of the reference RNA sequence; wherein the library represents variants comprising the single alteration of a single location, for at least 5%, at least 10%, at least 30%, at least 70%, or at least 90% 1% of the ribonucleotide locations of the reference RNA sequence.

114-116. (canceled)

117. The library of claim 113, wherein the reference RNA is a CRISPR guide RNA, and wherein the CRISPR guide RNA binds to CasX, CasY, Cas9, Cas12a, Cas12b, Cas12c, Cas12f, Cas12g, Cas12h, Cas12i, Cas12j, Cas13a, Cas13b, Cas13c, Cas13d, Cas14, CASCADE, CSM, or CSY.

118-120. (canceled)

121. A library comprising a plurality of protein variants, wherein each variant is independently a variant of the same reference protein, and each variant comprises an amino acid substitution, deletion, or insertion at one amino acid location of the reference protein sequence; wherein the library represents variants comprising the single alteration of a single location, for at least 5%, at least 10%, at least 30%, at least 70%, or at least 90% 1% of the amino acids of the reference protein sequence.

122-124. (canceled)

125. The library of 121, wherein the reference protein is a CRISPR associated protein, and wherein the CRISPR associated protein is CasX, CasY, Cas9, Cas12a, Cas12b, Cas12c, Cas12f, Cas12g, Cas12h, Cas12i, Cas12j, Cas13a, Cas13b, Cas13c, Cas13d, Cas14, CASCADE, CSM, or CSY.

126-131. (canceled)

132. A library comprising a plurality of DNA variants, wherein each variant is independently a variant of the same reference DNA, and each variant comprises a point mutation, deletion, or insertion at one deoxyribonucleotide location of the reference DNA sequence; wherein the library represents variants comprising the single alteration of a single location, for at least 5%, at least 10%, at least 30%, at least 70%, or at least 90% of the deoxyribonucleotide locations of the reference DNA sequence.

133-135. (canceled)
